(19) United States
(12) Patent Application Publication
(10) Pub. No.: US 2022/0310934 A1
(43) Pub. Date: Sep. 29, 2022

(54) COMPOUND INCLUDING NITROGEN EMITTING DEVICE INCLUDING THE SAME

(71) Applicant: Samsung Display Co., Ltd., Yongin-si (KR)

(72) Inventor: Takuya UNO, Yokohama (JP)

(21) Appl. No.: 17/675,394

(22) Filed: Feb. 18, 2022

Related U.S. Application Data
(63) Continuation-in-part of application No. 17/553,260, filed on Dec. 16, 2021, now abandoned.

(30) Foreign Application Priority Data
Feb. 18, 2021 (KR) 10-2021-0022037

(51) Int. Cl. H01L 51/00; C07D 491/048; C07F 7/08

(52) U.S. Cl. CPC H01L 51/0071; H01L 51/0072; C07D 491/048; H01L 51/0073; H01L 51/0094; C07F 7/0812; H01L 51/5012

(57) ABSTRACT

A light emitting device includes: a first electrode; a second electrode facing the first electrode; and a plurality of organic layers disposed between the first electrode and the second electrode, wherein at least one of the organic layers includes a compound including a nitrogen moiety, and the compound is of Formula 1 below:

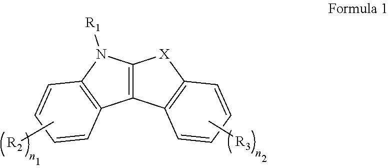

wherein, in Formula 1, the variables are defined herein.

### CROSS-REFERENCE TO RELATED APPLICATION

This application is a continuation-in-part of U.S. patent application Ser. No. 17/553,260, filed on Dec. 16, 2021, which claims priority from and the benefit of Korean Patent Application No. 10-2021-0022037, filed on Feb. 18, 2021, each of which is hereby incorporated by reference for all purposes as if fully set forth herein.

### BACKGROUND

### Field

Embodiments of the invention relate generally to a display devices, and more particularly, a novel nitrogen-containing compound and a light emitting device including the nitrogen-containing compound used as a hole transport material or a host material in an emission layer.

### Discussion of the Background

Recently, the use of an organic electroluminescence display apparatus as an image display apparatus is being actively developed. Unlike liquid crystal display apparatuses and the like, the organic electroluminescence display apparatus is a so-called self-luminescent display apparatus in which holes and electrons injected from a first electrode and a second electrode recombine in an emission layer, and thus a luminescent material including an organic compound in the emission layer emits light to implement display.

There is a demand for use of an organic electroluminescence device as a display apparatus having a low driving voltage, a high luminous efficiency, and a long service life, and the materials, for an organic electroluminescence device, capable of stably attaining such characteristics are being continuously developed. In addition, materials for use in a hole transport layer is being developed in order to realize a highly efficient organic electroluminescence device.

The above information disclosed in this Background section is only for understanding of the background of the inventive concepts, and, therefore, it may contain information that does not constitute prior art.

### SUMMARY

When light-emitting devices include nitrogen-containing compounds made according to principles and illustrative implementations of the invention, the light emitting devices have improved luminous efficiency. Thus, nitrogen-containing compounds made according to principles and illustrative implementations of the invention are capable of improving luminous efficiency and the lifespan of light emitting devices including such nitrogen-containing compounds.

Additional features of the inventive concepts will be set forth in the description which follows, and in part will be apparent from the description, or may be learned by practice of the inventive concepts.

According to one aspect of the invention, a light emitting device includes: a first electrode; a second electrode facing the first electrode; and a plurality of organic layers disposed between the first electrode and the second electrode, wherein at least one of the organic layers includes a compound including a nitrogen moiety, and the compound is of Formula 1 below:

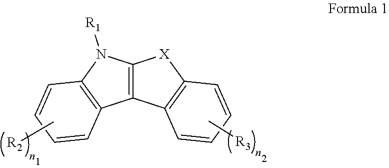

wherein, in Formula 1, the variables are defined herein.

The organic layers may include: a hole transport region disposed on the first electrode; an emission layer disposed on the hole transport region; and an electron transport region disposed on the emission layer, and the hole transport region may include the compound.

The hole transport region may include: a hole injection layer disposed on the first electrode; and a hole transport layer disposed on the hole injection layer, and the hole transport layer may include the compound.

The hole transport region may include a plurality of organic layers, and an organic layer adjacent to the emission layer may include the compound.

The organic layers may include: a hole transport region disposed on the first electrode; an emission layer disposed on the hole transport region; and an electron transport region disposed on the emission layer, and wherein the emission layer may include a host and a dopant, and the host may include the compound.

The nitrogen compound of Formula 1 may be any one of Formula 1-1 to Formula 1-3, as described herein.

The substituent of Formula 2-1 may be of Formula 2-1-1, as described herein.

The substituent of Formula 2-2 is any one of Formula 2-2-1 to Formula 2-2-4, as described herein

The groups L₁ and L₂ may each be, independently from one another, any one of Formula 3-1 to Formula 3-12, as described herein.

The groups R₁ to R₈ may each be, independently from one another, a hydrogen atom, a deuterium atom, a halogen atom, a substituted or unsubstituted silyl group, a substituted or unsubstituted methyl group, a substituted or unsubstituted t-butyl group, a substituted or unsubstituted phenyl group, a substituted or unsubstituted naphthyl group, a substituted or unsubstituted biphenyl group, a substituted or unsubstituted terphenyl group, a substituted or unsubstituted phenanthrenyl group, a substituted or unsubstituted carbazole group, a substituted or unsubstituted dibenzofuran group, a substituted or unsubstituted dibenzothiophene group, a substituted or unsubstituted phenoxazine group, or a substituted or unsubstituted acridine group.

In Formula 1, when R₁ is a substituent of Formula 2-1 or Formula 2-2, R₂ and R₃ may each be, independently from one another, a hydrogen atom, a deuterium atom, a halogen atom, a substituted or unsubstituted methyl group, or a substituted or unsubstituted phenyl group.

In Formula 1, when any one of R₂ or R₃ is a substituent of Formula 2-1 or Formula 2-2, R₁ may be a substituted or unsubstituted phenyl group, a substituted or unsubstituted biphenyl group, a substituted or unsubstituted terphenyl group, or a substituted or unsubstituted naphthyl group.

The light emitting device may further include a capping layer disposed on the 20 second electrode, wherein the capping layer has a refractive index of about 1.6 or more.

The compound of Formula 1 may include at least one compound of Compound Group 1 to Compound Group 3, as described herein.

According to another aspect of the invention, a compound includes a nitrogen moiety of Formula 1 below:

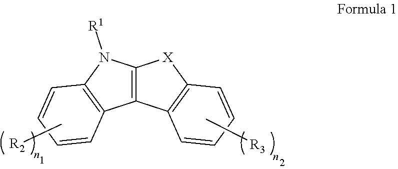

wherein, in formula 1, the variables are described herein.

The compound of Formula 1 may be of any one of Formula 1-1 to Formula 1-3, as described herein.

The substituent of Formula 2-1 may be of Formula 2-1-1, as described herein.

The substituent of Formula 2-2 may be any one of Formula 2-2-1 to Formula 2-2-4, as described herein.

The groups L₁ and L₂ may each be, independently from one another, any one of Formula 3-1 to Formula 3-12, as described herein.

The compound of Formula 1 may include at least one compound of Compound Group 1 to Compound Group 3, as described herein.

It is to be understood that both the foregoing general description and the following detailed description are illustrative and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate illustrative embodiments of the invention, and together with the description serve to explain the inventive concepts.

FIG. 1 is a plan view of an embodiment of a display apparatus constructed according to the principles of the invention.

FIG. 2 is a cross-sectional view taken along line I-I′ of FIG. 1.

FIG. 3 is a schematic cross-sectional view of an embodiment of a light emitting device constructed according to the principles of the invention.

FIG. 4 is a schematic cross-sectional view of another embodiment of a light emitting device constructed according to the principles of the invention.

FIG. 5 is a schematic cross-sectional view of a further embodiment of a light emitting device constructed according to the principles of the invention.

FIG. 6 is a schematic cross-sectional view of yet another embodiment of a light emitting device constructed according to the principles of the invention.

FIG. 7 is a cross-sectional view of another embodiment of a display apparatus including a light emitting device constructed according to the principles of the invention.

FIG. 8 is a cross-sectional view of a further embodiment of a display apparatus including a light emitting device constructed according to the principles of the invention.

### DETAILED DESCRIPTION

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing illustrative features of varying detail of some ways in which the inventive concepts may be implemented in practice. Therefore, unless otherwise specified, the features, components, modules, layers, films, panels, plates, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements, and repetitive explanations are omitted to avoid redundancy.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z-axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/of” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated fixed numbers, features, integers, steps, operations, elements, components, and/or groups thereof, but do not preclude the presence or addition of one or more other fixed numbers, features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various embodiments are described herein with reference to sectional and/or exploded illustrations that are schematic illustrations of idealized embodiments and/or intermediate structures. As such, variations from the shapes of the illustrations as a result, for example, of manufacturing techniques and/or tolerances, are to be expected. Thus, embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

### Definitions

As used herein, the term “atom” may mean an element or its corresponding radical bonded to one or more other atoms.

The terms “hydrogen” and “deuterium” refer to their respective atoms and corresponding radicals with the deuterium radical abbreviated “-D”, and the terms “—F, —Cl, —Br, and —I” are radicals of, respectively, fluorine, chlorine, bromine, and iodine.

The abbreviation “equiv” means “mole equivalent”.

As used herein, the term “substituted or unsubstituted” may mean being substituted or unsubstituted with at least one substituent selected from the group consisting of a deuterium atom, a halogen atom, a nitro group, a silyl group, an oxy group, a thio group, a sulfinyl group, a sulfonyl group, a carbonyl group, a boron group, a phosphine group, a phosphine oxide group, a phosphine sulfide group, an alkyl group, an alkenyl group, an alkynyl group, an alkoxy group, a hydrocarbon ring group, an aryl group, and a heterocyclic group. In addition, each of the substituents exemplified above may be substituted or unsubstituted. For example, a biphenyl group may be interpreted as an aryl group or a phenyl group substituted with a phenyl group.

As used herein, the phrase “bonded to an adjacent group to form a ring” may indicate that one is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In addition, the rings formed by being bonded to each other may be connected to another ring to form a spiro structure.

As used herein, the term “adjacent group” may mean a substituent substituted for an atom which is directly bonded to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In addition, two methyl groups in 4,5-dimethylphenanthrene may be interpreted as “adjacent groups” to each other. In addition, in 1,13-dimethylquinolino[3,2,1-de]acridine-5,9-dione, two methyl groups connected to the 1-position carbon and the 13-position carbon, respectively, may be interpreted as “adjacent groups” to each other.

As used herein, examples of the halogen atom may include a fluorine atom, a chlorine atom, a bromine atom, or an iodine atom.

As used herein, the alkyl group may be a linear, branched or cyclic type. The number of carbons in the alkyl group is 1 to 50, 1 to 30, 1 to 20, 1 to 10, or 1 to 6. Examples of the alkyl group may include a methyl group, an ethyl group, an n-propyl group, an isopropyl group, an n-butyl group, a s-butyl group, a t-butyl group, an i-butyl group, a 2-ethylbutyl group, a 3,3-a dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but are not limited thereto.

As used herein, the hydrocarbon ring group means any functional group or substituent derived from an aliphatic hydrocarbon ring. The hydrocarbon ring group may be a saturated hydrocarbon ring group having 5 to 30 or 5 to 20 ring-forming carbon atoms.

As used herein, the aryl group means any functional group or substituent derived from an aromatic hydrocarbon ring. The aryl group may be a monocyclic aryl group or a polycyclic aryl group. The number of ring-forming carbon atoms in the aryl group may be 6 to 60, 6 to 30, 6 to 20, or 6 to 15. Examples of the aryl group may include a phenyl group, a naphthyl group, a fluorenyl group, an anthracenyl group, a phenanthrenyl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, etc., but the embodiments are not limited thereto.

As used herein, the fluorenyl group may be substituted, and two substituents may be bonded to each other to form a spiro structure. Examples of cases where the fluorenyl group is substituted are as follows. However, the embodiments are not limited thereto.

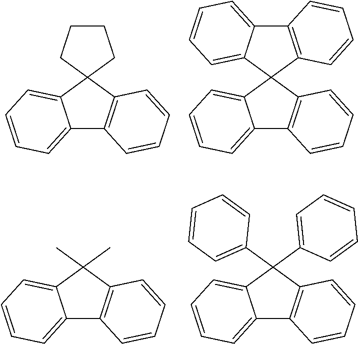

As used herein, the heterocyclic group means any functional group or substituent derived from a ring including at least one of B, O, N, P, Si, or Se as a heteroatom. The heterocyclic group includes an aliphatic heterocyclic group and an aromatic heterocyclic group. The aromatic heterocyclic group may be a heteroaryl group. The aliphatic heterocycle and the aromatic heterocycle may be monocyclic or polycyclic.

As used herein, the heterocyclic group may include at least one of B, O, N, P, Si or S as a heteroatom. If the heterocyclic group includes two or more heteroatoms, the two or more heteroatoms may be the same as or different from each other. The heterocyclic group may be a monocyclic heterocyclic group or a polycyclic heterocyclic group and has the concept including a heteroaryl group. The number of ring-forming carbon atoms in the heterocyclic group may be 2 to 60, 2 to 30, 2 to 20, or 2 to 10.

As used herein, the aliphatic heterocyclic group may include one or more among B, O, N, P, Si, and S as a heteroatom. The number of ring-forming carbon atoms of the aliphatic heterocyclic group may be 2 to 60, 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., but the embodiments are not limited thereto.

The heteroaryl group herein may include at least one of B, O, N, P, Si, or S as a heteroatom. When the heteroaryl group contains two or more heteroatoms, the two or more heteroatoms may be the same as or different from each other. The heteroaryl group may be a monocyclic heteroaryl group or a polycyclic heteroaryl group. The number of ring-forming carbon atoms of the heteroaryl group may be 2 to 60, 2 to 30, 2 to 20, or 2 to 10. Examples of the heteroaryl group may include a thiophene group, a furan group, a pyrrole group, an imidazole group, a triazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzoimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but the embodiments are not limited thereto.

As used herein, the above description of the aryl group may be applied to an arylene group except that the arylene group is a divalent group. The above description of the heteroaryl group may be applied to a heteroarylene group except that the heteroarylene group is a divalent group.

As used herein, the alkenyl group may be linear or branched. The number of carbon atoms in the alkynyl group is not specifically limited, but is 2 to 30, 2 to 20, or 2 to 10. Examples of the alkenyl group include a vinyl group, a 1-butenyl group, a 1-pentenyl group, a 1,3-butadienyl aryl group, a styrenyl group, a styrylvinyl group, etc., without limitation.

As used herein, the number of carbon atoms in the alkynyl group is not specifically limited, but is 2 to 30, 2 to 20, or 2 to 10. Examples of the alkynyl group include a vinyl group, a 2-butynyl group, a 2-pentynyl group, a 1,3-pentadiynyl aryl group, etc., but the embodiments are not limited thereto.

As used herein, the description of the alkyl group, the alkenyl group, the alkynyl group, the aryl group, and the heteroaryl group as described above may be equally applied to an alkyl linking group, an alkenyl linking group, an alkynyl linking group, an aryl group, and a heteroaryl group, respectively, except that the alkyl linking group, the alkenyl linking group, the alkynyl linking group, the aryl group, and the heteroaryl group are a divalent group, a trivalent group, or a tetravalent group.

As used herein, the silyl group includes an alkyl silyl group and an aryl silyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., but the embodiments of the invention are not limited thereto.

As used herein, the number of ring-forming carbon atoms in the carbonyl group is not specifically limited, but may be 1 to 40, 1 to 30, or 1 to 20. For example, the carbonyl group may have the following structures, but the embodiments are not limited thereto.

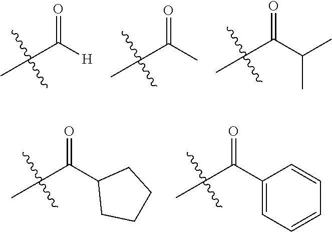

As used herein, the number of carbon atoms in the sulfinyl group and sulfonyl group is not particularly limited, but may be 1 to 30. The sulfinyl group may include an alkyl sulfinyl group and an aryl sulfinyl group. The sulfonyl group may include an alkyl sulfonyl group and an aryl sulfonyl group.

As used herein, a thiol group may include an alkylthio group and an arylthio group. The thiol group may mean that a sulfur atom is bonded to the alkyl group or the aryl group as defined above. Examples of the thiol group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but the embodiments are not limited thereto.

The oxy group herein may mean that an oxygen atom is bonded to the alkyl group or the aryl group as defined above. The oxy group may include an alkoxy group and an aryl oxy group. The alkoxy group may be a linear chain, a branched chain or a ring chain. The number of carbon atoms in the alkoxy group is not specifically limited, but may be, for example, 1 to 20 or 1 to 10. Examples of the oxy group include methoxy, ethoxy, n-propoxy, isopropoxy, butoxy, pentyloxy, hexyloxy, octyloxy, nonyloxy, decyloxy, benzyloxy, etc., but the embodiments are not limited thereto.

The boron group herein may mean that a boron atom is bonded to the alkyl group or the aryl group as defined above. The boron group includes an alkyl boron group and an aryl boron group. Examples of the boron group may include a trimethylboron group, a triethylboron group, a t-butyldimethylboron group, a triphenylboron group, a diphenylboron group, a phenylboron group, etc., but the embodiments are not limited thereto.

As used herein, the number of carbon atoms in an amine group is not specifically limited, but may be 1 to 30. The amine group may include an alkyl amine group and an aryl amine group. Examples of the amine group may include a methylamine group, a dimethylamine group, a phenylamine group, a diphenylamine group, a naphthylamine group, a 9-methyl-anthracenylamine group, a triphenylamine group, etc., but the embodiments are not limited thereto.

As used herein, the alkyl group among an alkylthio group, an alkyl sulfoxy group, an alkyl aryl group, an alkyl amine group, an alkyl boron group, and an alkyl silyl group is the same as the examples of the alkyl group described above.

As used herein, the aryl group among an aryloxy group, an arylthio group, an aryl sulfoxy group, an aryl amine group, an aryl boron group, and an aryl silyl group is the same as the examples of the aryl group described above.

As used herein, a direct linkage herein may mean a single bond.

As used herein,

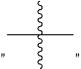

and “ [symbol] ” mean a position to be connected.

Hereinafter, embodiments are described with reference to the accompanying drawings.

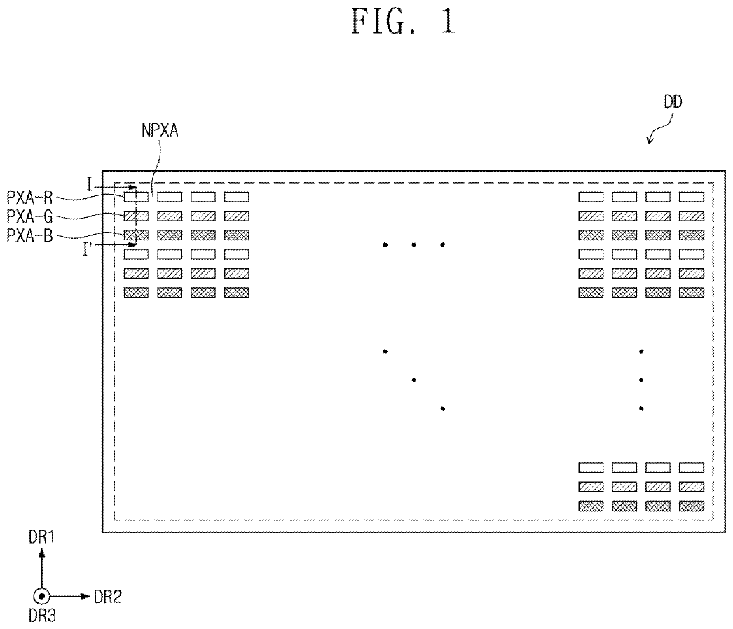

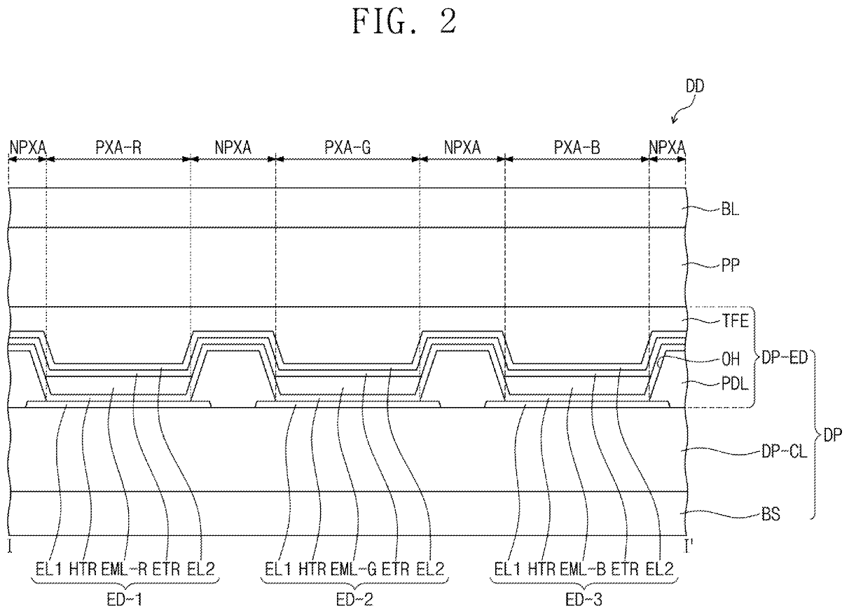

FIGS. 1-2

FIG. 1 is a plan view of an embodiment of a display apparatus constructed according to the principles of the invention. FIG. 2 is a cross-sectional view taken along line I-I′ of FIG. 1. FIG. 2 is a cross-sectional view illustrating a part taken along line I-I′ of FIG. 1.

The display apparatus DD may include a display panel DP and an optical layer PP disposed on the display panel DP. The display panel DP includes light emitting devices ED-1, ED-2, and ED-3. The display apparatus DD may include a plurality of light emitting devices ED-1, ED-2, and ED-3. The optical layer PP may be disposed on the display panel DP and control reflected light in the display panel DP due to external light. The optical layer PP may include, for example, a polarization layer or a color filter layer. Unlike the view illustrated in FIG. 2, the optical layer PP may be omitted from the display apparatus DD.

An upper base layer BL may be disposed on the optical layer PP. The upper base layer BL may be a member which provides a base surface on which the optical layer PP disposed. The upper base layer BL may be a glass substrate, a metal substrate, a plastic substrate, etc. However, the embodiments of the invention are not limited thereto, and the upper base layer BL may be an inorganic layer, an organic layer, or a composite material layer. In addition, unlike shown, in an embodiment, the upper base layer BL may be omitted.

The display apparatus DD according to an embodiment may further include a filling layer. The filling layer may be disposed between a display device layer DP-ED and the upper base layer BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and a display device layer DP-ED. The display device layer DP-ED may include a pixel defining film PDL, the light emitting devices ED-1, ED-2, and ED-3 disposed between portions of the pixel defining film PDL, and an encapsulation layer TFE disposed on the light emitting devices ED-1, ED-2, and ED-3.

The base layer BS may be a member which provides a base surface on which the display device layer DP-ED is disposed. The base layer BS may be a glass substrate, a metal substrate, a plastic substrate, etc. However, the embodiments of the invention are not limited thereto, and the base layer BS may be an inorganic layer, an organic layer, or a composite material layer.

In an embodiment, the circuit layer DP-CL is disposed on the base layer BS, and the circuit layer DP-CL may include a plurality of transistors. Each of the transistors may include a control electrode, an input electrode, and an output electrode. For example, the circuit layer DP-CL may include a switching transistor and a driving transistor in order to drive the light emitting devices ED-1, ED-2, and ED-3 of the display device layer DP-ED.

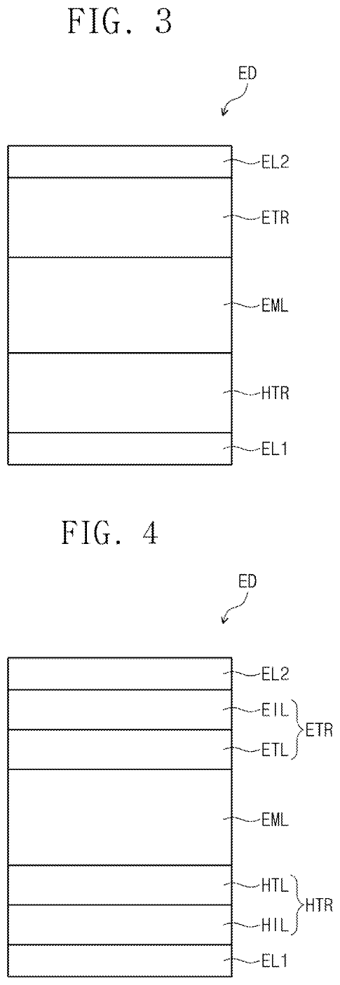

Each of the light emitting devices ED-1, ED-2, and ED-3 may have a structure of a light emitting device ED according to FIGS. 3 to 6, which will be described below. Each of the light emitting devices ED-1, ED-2 and ED-3 may include a first electrode EL1, a hole transport region HTR, emission layers EML-R, EML-G and EML-B, an electron transport region ETR, and a second electrode EL2.

FIG. 2 illustrates an embodiment in which the emission layers EML-R, EML-G, and EML-B of the light emitting devices ED-1, ED-2, and ED-3 are disposed in the openings OH defined in the pixel defining film PDL, and the hole transport region HTR, the electron transport region ETR, and the second electrode EL2 are provided as a common layer in the entire light emitting devices ED-1, ED-2, and ED-3. However, the embodiments of the invention are not limited thereto, and unlike the feature illustrated in FIG. 2, the hole transport region HTR and the electron transport region ETR may be provided by being patterned inside the opening hole OH defined in the pixel defining film PDL. For example, the hole transport region HTR, the emission layers EML-R, EML-G, and EML-B, and the electron transport region ETR of the light emitting devices ED-1, ED-2, and ED-3 may be provided by being patterned in an inkjet printing method.

The encapsulation layer TFE may cover the light emitting devices ED-1, ED-2 and ED-3. The encapsulation layer TFE may seal the display device layer DP-ED. The encapsulation layer TFE may be a thin film encapsulation layer. The encapsulation layer TFE may be formed by laminating one layer or a plurality of layers. The encapsulation layer TFE includes at least one insulation layer. The encapsulation layer TFE may include at least one inorganic film (hereinafter, an encapsulation-inorganic film). The encapsulation layer TFE may also include at least one organic film (hereinafter, an encapsulation-organic film) and at least one encapsulation-inorganic film.

The encapsulation-inorganic film protects the display device layer DP-ED from at least one of moisture and oxygen, and the encapsulation-organic film protects the display device layer DP-ED from foreign substances such as dust particles. The encapsulation-inorganic film may include a silicon nitride, a silicon oxynitride, a silicon oxide, a titanium oxide, an aluminum oxide, or the like, but the embodiments of the invention are not particularly limited thereto. The encapsulation-organic film may include an acrylic-based compound, an epoxy-based compound, or the like. The encapsulation-organic film may include a photopolymerizable organic material, but the embodiments of the invention are not particularly limited thereto. The encapsulation layer TFE may be disposed on the second electrode EL2 and may be disposed filling the opening hole OH.

Referring to FIGS. 1 and 2, the display apparatus DD may include a non-light emitting region NPXA and light emitting regions PXA-R, PXA-G and PXA-B. The light emitting regions PXA-R, PXA-G and PXA-B each may be a region which emits light generated from the light emitting devices ED-1, ED-2 and ED-3, respectively. The light emitting regions PXA-R, PXA-G, and PXA-B may be spaced apart from each other in a plane.

Each of the light emitting regions PXA-R, PXA-G, and PXA-B may be a region divided by the pixel defining film PDL. The non-light emitting regions NPXA may be regions between the adjacent light emitting regions PXA-R, PXA-G, and PXA-B, which correspond to portions of the pixel defining film PDL. As described herein, each of the light emitting regions PXA-R, PXA-G, and PXA-B may correspond to a pixel. The pixel defining film PDL may separate the light emitting devices ED-1, ED-2, and ED-3. The emission layers EML-R, EML-G and EML-B of the light emitting devices ED-1, ED-2 and ED-3 may be disposed in openings OH defined by the pixel defining film PDL and separated from each other.

The light emitting regions PXA-R, PXA-G and PXA-B may be divided into a plurality of groups according to the color of light generated from the light emitting devices ED-1, ED-2 and ED-3. In the display apparatus DD shown in FIGS. 1 and 2, three light emitting regions PXA-R, PXA-G, and PXA-B which emit red light, green light, and blue light, respectively are exemplarily illustrated. For example, the display apparatus DD may include the red light emitting region PXA-R, the green light emitting region PXA-G, and the blue light emitting region PXA-B which are separated from one another.

In the display apparatus DD, the plurality of light emitting devices ED-1, ED-2 and ED-3 may emit light beams having wavelengths different from one another. For example, in an embodiment, the display apparatus DD may include a first light emitting device ED-1 that emits red light, a second light emitting device ED-2 that emits green light, and a third light emitting device ED-3 that emits blue light. That is, the red light emitting region PXA-R, the green light emitting region PXA-G, and the blue light emitting region PXA-B of the display apparatus DD may correspond to the first light emitting device ED-1, the second light emitting device ED-2, and the third light emitting device ED-3, respectively.

However, the embodiments of the invention are not limited thereto, and the first to third light emitting devices ED-1, ED-2, and ED-3 may emit light beams in the same wavelength range or at least one light emitting device may emit a light beam in a wavelength range different from the others. For example, the first to third light emitting devices ED-1, ED-2, and ED-3 may all emit blue light.

The light emitting regions PXA-R, PXA-G, and PXA-B in the display apparatus DD may be arranged in a generally elongated (stripe) form. Referring to FIG. 1, the plurality of red light emitting regions PXA-R, the plurality of green light emitting regions PXA-G, and the plurality of blue light emitting regions PXA-B each may be arranged along a second directional axis DR2. In addition, the red light emitting region PXA-R, the green light emitting region PXA-G, and the blue light emitting region PXA-B may be alternately arranged in this order along a first directional axis DR1.

FIGS. 1 and 2 illustrate that all the light emitting regions PXA-R, PXA-G, and PXA-B have similar areas, but the embodiments of the invention are not limited thereto, and the light emitting regions PXA-R, PXA-G, and PXA-B may have different areas from each other according to a wavelength range of the emitted light. In this case, the areas of the light emitting regions PXA-R, PXA-G, and PXA-B may mean areas when viewed in a plane defined by the first directional axis DR1 and the second directional axis DR2.

The configuration of the light emitting regions PXA-R, PXA-G, and PXA-B is not limited to the features illustrated in FIG. 1, and the order in which the red light emitting region PXA-R, the green light emitting region PXA-G, and the blue light emitting region PXA-B are arranged may be variously combined and provided according to characteristics of a display quality required in the display apparatus DD. For example, the configuration of the light emitting regions PXA-R, PXA-G, and PXA-B may be a configuration sold under the trade designation PenTile matrix by Samsung Display Co., Ltd. of Gyeonggi-do, Republic of Korea, or a diamond configuration.

In addition, the areas of the light emitting regions PXA-R, PXA-G, and PXA-B may be different from each other. For example, in an embodiment, the area of the green light emitting region PXA-G may be smaller than that of the blue light emitting region PXA-B, but the embodiments are not limited thereto.

FIGS. 3-6

FIGS. 3 to 6 are cross-sectional views schematically illustrating organic electroluminescence devices according to embodiments. Referring to FIGS. 3 to 6, in each of light emitting devices ED, a first electrode EL1 and a second electrode EL2 are disposed facing each other, and a plurality of organic layers may be disposed between the first electrode EL1 and the second electrode EL2. The plurality of organic layers may include a hole transport region HTR, an emission layer EML, and an electron transport region ETR. That is, each of the light emitting devices ED according to embodiments may include the first electrode EL1, the hole transport region HTR, the emission layer EML, the electron transport region ETR, and the second electrode EL2 that are sequentially stacked. A capping layer CPL may be further disposed on the second electrode EL2.

Each of the light emitting devices ED may include a nitrogen-containing compound described below in at least one organic layer among the plurality of organic layers disposed between the first electrode EL1 and the second electrode EL2. For example, each of the light emitting devices ED may include a nitrogen-containing compound described below in the hole transfer region HTR disposed between the first electrode EL1 and the second electrode EL2. However, the embodiments of the invention are not limited thereto, and each of the light emitting devices ED may include a nitrogen-containing compound according to an embodiment described below in at least one organic layer which is included in the emission layer EML and the electron transfer region ETR which are the plurality of organic layers disposed between the first electrode EL1 and the second electrode EL2, or may include a nitrogen-containing compound according to an embodiment described below in the capping layer CPL disposed on the second electrode EL2.

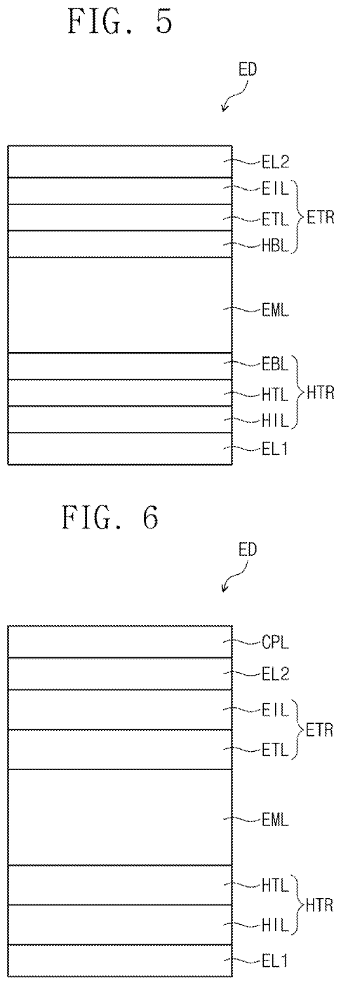

FIG. 3 is a schematic cross-sectional view of an embodiment of a light emitting device constructed according to the principles of the invention. FIG. 4 is a schematic cross-sectional view of another embodiment of a light emitting device constructed according to the principles of the invention. FIG. 5 is a schematic cross-sectional view of a further embodiment of a light emitting device constructed according to the principles of the invention.

Compared to FIG. 3, FIG. 4 illustrates a cross-sectional view of a light emitting device ED, in which a hole transport region HTR includes a hole injection layer HIL and a hole transport layer HTL, and an electron transport region ETR includes an electron injection layer EIL and an electron transport layer ETL. In addition, compared to FIG. 3, FIG. 5 illustrates a cross-sectional view of a light emitting device ED of an embodiment, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared to FIG. 4, FIG. 6 illustrates a cross-sectional view of a light emitting device ED including a capping layer CPL disposed on a second electrode EL2. FIG. 6 is a schematic cross-sectional view of yet another embodiment of a light emitting device constructed according to the principles of the invention.

The first electrode EL1 has conductivity. The first electrode EL1 may be formed of a metal material, a metal alloy, or a conductive compound. The first electrode EL1 may be an anode or a cathode. However, the embodiments of the invention are not limited thereto. In addition, the first electrode EL1 may be a pixel electrode. The first electrode EL1 may be a transmissive electrode, a transflective electrode, or a reflective electrode. If the first electrode EL1 is the transmissive electrode, the first electrode EL1 may be formed using a transparent metal oxide such as an indium tin oxide (ITO), an indium zinc oxide (IZO), a zinc oxide (ZnO), and an indium tin zinc oxide (ITZO). If the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca, LiF/Al, Mo, Ti, W, In, Zn, Sn, a compound thereof or a mixture thereof (e.g., a mixture of Ag and Mg). Alternatively, the first electrode EL1 may have a multilayer structure including a reflective film or a transflective film formed of the above-described materials, and a transparent conductive film formed of the ITO, IZO, ZnO, ITZO, etc. For example, the first electrode EL1 may have a three-layer structure of an ITO/Ag/ITO, but the embodiments of the invention are not limited thereto. In addition, although the embodiments of the invention are not limited thereto, the first electrode EL1 may include the above-described metal materials, combinations of at least two metal materials of the above-described metal materials, oxides of the above-described metal materials, or the like. The thickness of the first electrode EL1 may be from about 700 Å to about 10,000 Å. For example, the thickness of the first electrode EL1 may be from about 1,000 Å to about 3,000 Å.

The hole transport region HTR is provided on the first electrode EL1. The hole transport region HTR may include at least one of a hole injection layer HIL, a hole transport layer HTL, a buffer layer or an emission-auxiliary layer, or an electron blocking layer EBL. The thickness of the hole transport region HTR may be, for example, from about 50 Å to about 15,000 Å. The hole transport region HTR may have a single layer formed of a single material, a single layer formed of a plurality of different materials, or a multilayer structure including a plurality of layers formed of a plurality of different materials.

For example, the hole transport region HTR may have a single layer structure of the hole injection layer HIL or the hole transport layer HTL, and may have a single layer structure formed of a hole injection material and a hole transport material. In addition, the hole transport region HTR may have a single layer structure formed of a plurality of different materials, or a structure in which a hole injection layer HIL/hole transport layer HTL, a hole injection layer HIL/hole transport layer HTL/buffer layer, a hole injection layer HIL/buffer layer, a hole transport layer HTL/buffer layer, or a hole injection layer HIL/hole transport layer HTL/electron blocking layer EBL are stacked in order from the first electrode EL1, but the embodiments of the invention are not limited thereto.

The hole transport region HTR may be formed using various methods such as a vacuum deposition method, a spin coating method, a cast method, a Langmuir-Blodgett (LB) method, an inkjet printing method, a laser printing method, and a laser induced thermal imaging (LITI) method.

The hole transport region HTR in the light emitting device ED may include a nitrogen-containing compound made according to an embodiment of the invention. The nitrogen-containing compound may be a compound not including an amine group and a cyano group in the molecular structure.

The nitrogen-containing compound may include a carbazole substituent linked to a benzofuroindole or a benzothienoindole skeleton. The nitrogen-containing compound is linked, via a nitrogen atom of a carbazole substituent, to a benzofuroindole or a benzothienoindole skeleton, and a linker may be disposed between the carbazole substituent and the benzofuroindole or the benzothienoindole skeleton. Alternatively, the nitrogen-containing compound is linked, via one carbon atom in the benzene ring of a carbazole substituent to a benzofuroindole or benzothienoindole skeleton, and a linker may be disposed between the carbazole substituent and the benzofuroindole or benzothienoindole skeleton, or the carbazole substituent may be directly bonded to the benzofuroindole or benzothienoindole skeleton.

The nitrogen-containing compound may be represented by Formula 1 below:

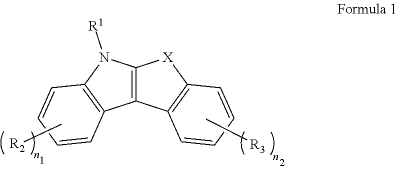

In Formula 1, X is O or S. When X is O, the nitrogen-containing compound may have a benzofuroindole skeleton. When X is S, the nitrogen-containing compound may have a benzothienoindole skeleton. The nitrogen-containing compound may include only one benzofuroindole group or one benzothienoindole group in the molecular structure. That is, the nitrogen-containing compound may not include a benzofuroindole group or a benzothienoindole group as a substituent other than a benzofuroindole skeleton or a benzothienoindole skeleton shown in Formula 1.

In Formula 1, R₁ to R₃ are each independently a hydrogen atom, a deuterium atom, a halogen atom, a nitro group, a substituted or unsubstituted silyl group, a substituted or unsubstituted boron group, a substituted or unsubstituted oxy group, a substituted or unsubstituted thio group, a substituted or unsubstituted carbonyl group, a substituted or unsubstituted alkyl group having 1 to 30 carbon atoms, a substituted or unsubstituted aryl group having 6 to 60 ring-forming carbon atoms, or a substituted or unsubstituted heteroaryl group having 2 to 60 ring-forming carbon atoms. For example, R₁ to R₃ may be each independently a hydrogen atom, a deuterium atom, a halogen atom, a substituted or unsubstituted silyl group, a substituted or unsubstituted methyl group, a substituted or unsubstituted t-butyl group, a substituted or unsubstituted phenyl group, a substituted or unsubstituted naphthyl group, a substituted or unsubstituted biphenyl group, a substituted or unsubstituted terphenyl group, a substituted or unsubstituted phenanthrenyl group, a substituted or unsubstituted carbazole group, a substituted or unsubstituted dibenzofuran group, a substituted or unsubstituted dibenzothiophene group, a substituted or unsubstituted phenoxazine group, or a substituted or unsubstituted acridine group.

In Formula 1, n₁ and n₂ are each independently an integer of 1 to 4. In Formula 1, if n₁ is an integer of 2 or more, a plurality of R₂'s may be the same as or different from each other. In Formula 1, if n₂ is an integer of 2 or more, a plurality of R₃'s may be the same as or different from each other.

Any one among R₁ to R₃ in Formula 1 is a substituent represented by Formula 2-1 below or a substituent represented by Formula 2-2 below:

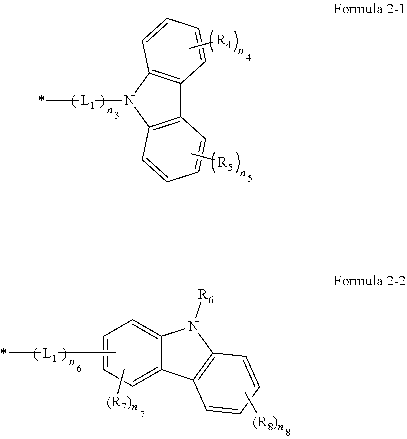

In Formula 2-1 and Formula 2-2, L₁ and L₂ are each independently a substituted or unsubstituted silyl linking group, a substituted or unsubstituted arylene group having 6 to 60 ring-forming carbon atoms, or a substituted or unsubstituted heteroarylene group having 2 to 60 ring-forming carbon atoms. For example, L₁ and L₂ may be each independently a substituted or unsubstituted silyl linking group, a substituted or unsubstituted phenylene group, a substituted or unsubstituted naphthyl linking group, a substituted or unsubstituted fluorenyl linking group, a substituted or unsubstituted phenanthrenyl linking group, a substituted or unsubstituted carbazole linking group, a substituted or unsubstituted dibenzofuran linking group, a substituted or unsubstituted dibenzothiophene linking group, or a substituted or unsubstituted spiro acridine fluorenyl linking group.

The case when each of L₁ and L₂ is an electron-withdrawing compound linking group or a 2,2′-biphenyl linking group is excluded. That is, the case when each of L₁ and L₂ is a pyridine linking group, a pyrazine linking group, a quinoline linking group, a quinoxaline linking group, and a triazine linking group or is a structure of Formula a below is excluded.

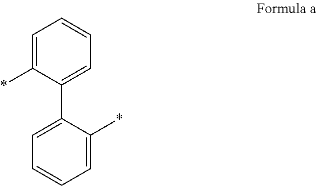 [SMILES: *c1ccccc1-c1ccccc1*]

Formula a illustrates that the 2,2′-biphenyl linking group does not include a substituent, but the case when a substituent is substituted at the 2,2′-biphenyl linking group structure in Formula 1 is excluded in L₁ and L₂.

In Formula 2-1, n₃ is an integer of 1 to 3. That is, if a substituent represented by Formula 2-1 is linked to a benzofuroindole skeleton or a benzothienoindole skeleton structure in Formula 1, the benzofuroindole skeleton or the benzothienoindole skeleton and the substituent, Formula 2-1, may be linked via linker L₁. If n₃ is an integer of 2 or more, a plurality of L₁'s may be the same as each other or at least one L₁ may be different from the others. For example, L₁'s may all be m-phenylene groups or p-phenylene groups. Alternatively, any one of a plurality of L₁'s may be a phenyl group, any one may be a carbazole linking group, a dibenzofuran linking group, or a dibenzothiophene linking group.

In Formula 2-2, n₆ is an integer of 0 to 3. The case where n₆ is 0 may mean a direct linkage without linker L₂. That is, if a substituent represented by Formula 2-2 is linked to a benzofuroindole skeleton or a benzothienoindole skeleton structure in Formula 1, the benzofuroindole skeleton or the benzothienoindole skeleton and the substituent, Formula 2-2, may be linked via linker L₂, or the substituent, Formula 2-2, may be directed linked to the benzofuroindole skeleton or the benzothienoindole skeleton. If n₆ is an integer of 2 or more, a plurality of L₂'s may be the same as each other or at least one L₂ may be different from the others. For example, L₂'s may all be m-phenylene groups or p-phenylene groups. Alternatively, any one of a plurality of L₂'s may be a phenyl group, any one may be a carbazole linking group, a dibenzofuran linking group, or a dibenzothiophene linking group.

In Formula 2-1 and Formula 2-2, R₄ to R₈ are each independently a hydrogen atom, a deuterium atom, a halogen atom, a nitro group, a substituted or unsubstituted silyl group, a substituted or unsubstituted boron group, a substituted or unsubstituted oxy group, a substituted or unsubstituted thio group, a substituted or unsubstituted carbonyl group, a substituted or unsubstituted alkyl group having 1 to 30 carbon atoms, a substituted or unsubstituted aryl group having 6 to 60 ring-forming carbon atoms, or a substituted or unsubstituted heteroaryl group having 2 to 60 ring-forming carbon atoms. For example, R₄ to R₈ may be each independently a hydrogen atom, a deuterium atom, a halogen atom, a substituted or unsubstituted silyl group, a substituted or unsubstituted methyl group, a substituted or unsubstituted t-butyl group, a substituted or unsubstituted phenyl group, a substituted or unsubstituted naphthyl group, a substituted or unsubstituted biphenyl group, a substituted or unsubstituted terphenyl group, a substituted or unsubstituted phenanthrenyl group, a substituted or unsubstituted carbazole group, a substituted or unsubstituted dibenzofuran group, a substituted or unsubstituted dibenzothiophene group, a substituted or unsubstituted phenoxazine group, or a substituted or unsubstituted acridine group.

In Formula 2-1 to Formula 2-2, n₄, n₅, and n₈ are each independently an integer of 1 to 4. The variable n₇ is an integer of 1 to 3. In Formula 2-1, if n₄ is an integer of 2 or more, a plurality of R₄'s may be the same as or different from each other. In Formula 2-1, if n₅ is an integer of 2 or more, a plurality of R₅'s may be the same as or different from each other. In Formula 2-2, if n₇ is an integer of 2 or more, a plurality of R₇'s are the same as or different from each other. In Formula 2-2, if n₈ is an integer of 2 or more, a plurality of R₈'s are the same as or different from each other.

In Formula 2-1 and Formula 2-2, [symbol] is a position linked to the benzofuroindole skeleton or benzothienoindole skeleton structure represented by Formula 1.

In the molecular structure of Formula 1, only one among R₁ to R₃ may include a substituent represented by Formula 2-1 or a carbazole substituent represented by Formula 2-2, and the other two may not include a carbazole moiety. For example, in Formula 1, R₁ may include a substituent represented by Formula 2-1 or a carbazole substituent represented by Formula 2-2, and R₂ and R₃ may not include a carbazole group. Alternatively, in Formula 1, R₂ may include a substituent represented by Formula 2-1 or a carbazole substituent represented by Formula 2-2, and R₁ and R₃ may not include a carbazole group. Alternatively, in Formula 1, R₃ may include a substituent represented by Formula 2-1 or a carbazole substituent represented by Formula 2-2, and R₁ and R₂ may not include a carbazole group.

In Formula 1, when R₁ is a substituent represented by Formula 2-1 or Formula 2-2 above, R₂ and R₃ may be each independently a hydrogen atom, a deuterium atom, a halogen atom, a substituted or unsubstituted alkyl group having 1 to 30 carbon atoms group, or a substituted or unsubstituted aryl group having 6 to 60 ring-forming carbon atoms group. For example, R₂ and R₃ may be each independently a hydrogen atom, a deuterium atom, a fluorine atom, a substituted or unsubstituted methyl group, or a substituted or unsubstituted phenyl group.

In Formula 1, when any one of R₂ or R₃ is a substituent represented by Formula 2-1 or Formula 2-2 above, R₁ may be a substituted or unsubstituted aryl group having 6 to 60 ring-forming carbon atoms. For example, R₁ may be a substituted or unsubstituted phenyl group, a substituted or unsubstituted biphenyl group, a substituted or unsubstituted terphenyl group, or a substituted or unsubstituted naphthyl group.

In Formula 1, if any a substituent represented by Formula 2-1 above is substituted in the nitrogen-containing compound represented by Formula 1 above, L₁ is a substituted or unsubstituted arylene group having 6 to 60 ring-forming carbon atoms, and n₃ is 1, that is, the substituent represented by Formula 2-1 is substituted in the nitrogen-containing compound represented by Formula 1 via linker, at least one among R₄ and R₅ is a substituent not a hydrogen atom. When the substituent represented by Formula 2-1 is substituted in the nitrogen-containing compound represented by Formula 1 via linker, at least one among R₄ and R₅ is a substituted or unsubstituted silyl group, a substituted or unsubstituted aryl group having 6 to 60 ring-forming carbon atoms, or a substituted or unsubstituted heteroaryl group having 2 to 60 ring-forming carbon atoms.

The nitrogen-containing compound may include a structure represented by Formula 1. The nitrogen-containing compound may have a structure including a carbazole substituent linked to a benzofuroindole or benzothienoindole skeleton. In particular, the nitrogen-containing compound may have a structure in which the carbazole substituent is linked to a benzofuroindole or benzothienoindole skeleton at the position of the nitrogen atom of the carbazole substituent via a linker. Alternatively, the nitrogen-containing compound may have a structure in which the carbazole substituent is linked to a benzofuroindole or benzothienoindole skeleton at the position of one carbon atom of the benzene rings of the carbazole substituent via a linker, or a structure in which the carbazole substituent is directly linked to a benzofuroindole or benzothienoindole skeleton at the position of one carbon atom of the benzene rings of the carbazole substituent. Accordingly, the nitrogen-containing compound may have improved hole transport ability of the entire molecule by means of the benzofuroindole or benzothienoindole skeleton, and the electron resistance and lowest triplet excitation energy may be increased, and thus when the nitrogen-containing compound is included in the hole transport layer of the light emitting device, high efficiency and a long service life of the light emitting device may be achieved.

In Formula 1, the substituent represented by Formula 2-1 may be represented by Formula 2-1-1 below:

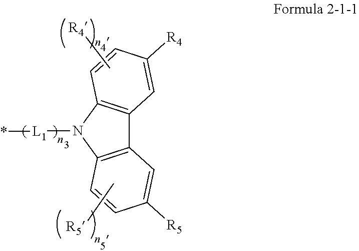

Formula 2-1-1 is the case where the position, at which R₄ and R₅ substituents are linked, is specified in the substituent represented by Formula 2-1.

In Formula 2-1-1, R₄‘ and R’ are each independently a hydrogen atom or a deuterium atom. In an embodiment, each of a plurality of R₄'s and R₅'s may be a hydrogen atom. Alternatively, each of a plurality of R₄'s and R₅'s may be a deuterium atom.

In Formula 2-1-1, n₄′ and n₅′ are each independently an integer of 1 to 3. In Formula 2-1-1, if n₄′ is an integer of 2 or more, a plurality of R₄'s may be the same as or different from each other. In Formula 2-1-1, if n₅′ is an integer of 2 or more, a plurality of R₅'s may be the same as or different from each other. In Formula 2-1-1, those described in Formula 2-1 above may be equally applied to L₁, n₃, R₄ and R₅.

In Formula 1, the substituent represented by Formula 2-2 may be represented by any one among Formula 2-2-1 to Formula 2-2-4 below:

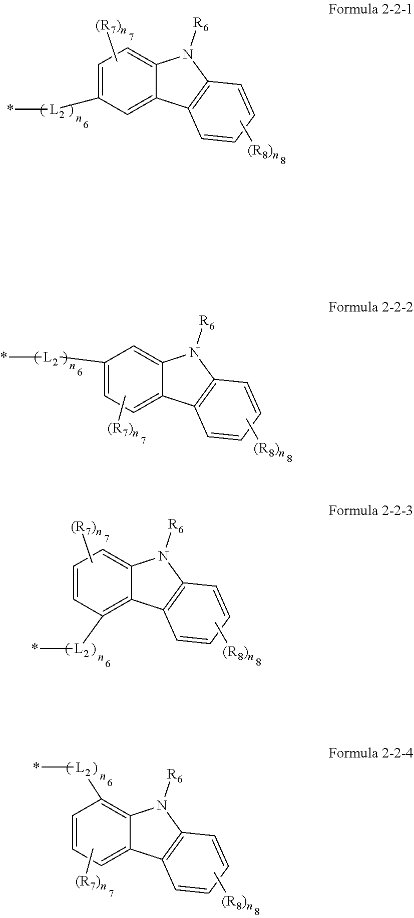

Formula 2-2-1 to Formula 2-2-4 are the cases where the linking group represented by L₂ and the position linked to the skeletal structure of Formula 1 are specified in the substituent represented by Formula 2-2. In Formula 2-2-1 to Formula 2-2-4, those described in Formula 2-2 above may be equally applied to L₂, R₆ to R₈, and n₆ to n₈.

In Formula 2-1 and Formula 2-2, L₁ and L₂ may be each independently represented by any one among Formula 3-1 to Formula 3-3 below:

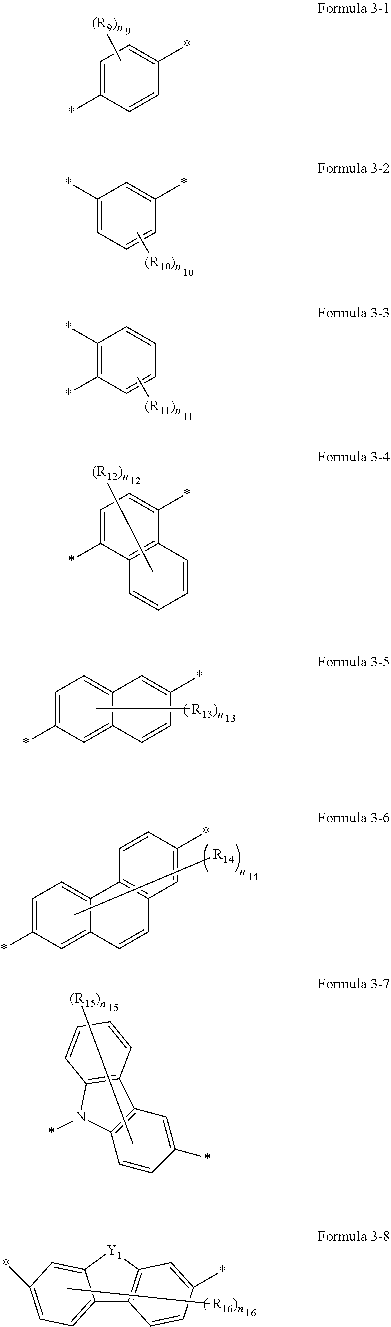

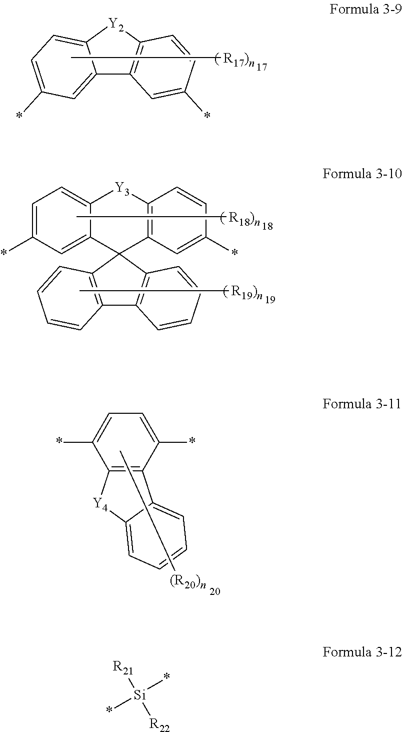

Formula 3-1 to Formula 3-12 are the cases where the structure of a linker which connects the skeletal structure of Formula 1 with the carbazole moiety of the substituents represented by Formula 2-1 and Formula 2-2 is specified. In Formula 3-1 to Formula 3-12, Y₁ to Y₄ are each independently NR_{b}, O, or S.

In Formula 3-1 and Formula 3-12, R_{b} and R₉ to R₂₂ are each independently a hydrogen atom, a deuterium atom, a halogen atom, a nitro group, a substituted or unsubstituted silyl group, a substituted or unsubstituted boron group, a substituted or unsubstituted oxy group, a substituted or unsubstituted thio group, a substituted or unsubstituted carbonyl group, a substituted or unsubstituted alkyl group having 1 to 30 carbon atoms, a substituted or unsubstituted aryl group having 6 to 60 ring-forming carbon atoms, or a substituted or unsubstituted heteroaryl group having 2 to 60 ring-forming carbon atoms. For example, R_{b} and R₉ to R₂₂ may be each independently a hydrogen atom, a deuterium atom, or a substituted or unsubstituted phenyl group.

In Formula 3-1 to Formula 3-12, n₉ to n₁₁ are each independently an integer of 1 to 4. The variables n₁₂ and n₁₃, n₁₅ to n₁₈, and n₂₀ are each independently an integer of 1 to 3. The variables n₁₄ and n₁₉ are each independently an integer of 1 to 8. In Formula 3-1, if n₉ is an integer of 2 or more, a plurality of R₉'s may be the same as or different from each other. In Formula 3-2, if n₁₀ is an integer of 2 or more, a plurality of R₁₀'s may be the same as or different from each other. In Formula 3-3, if n₁₁ is an integer of 2 or more, a plurality of R₁₁'s are the same as or different from each other. In Formula 3-4, if n₁₂ is an integer of 2 or more, a plurality of R₁₂'s are the same as or different from each other. In Formula 3-5, if n₁₃ is an integer of 2 or more, a plurality of R₁₃'s are the same as or different from each other. In Formula 3-6, if n₁₄ is an integer of 2 or more, a plurality of R₁₄'s are the same as or different from each other. In Formula 3-7, if n₁₅ is an integer of 2 or more, a plurality of R₁₅'s are the same as or different from each other. In Formula 3-8, if n₁₆ is an integer of 2 or more, a plurality of R₁₆'s are the same as or different from each other. In Formula 3-9, if n₁₇ is an integer of 2 or more, a plurality of R₁₇'s are the same as or different from each other. In Formula 3-10, if n₁₈ is an integer of 2 or more, a plurality of R₁₈'s are the same as or different from each other. In Formula 3-10, if n₁₉ is an integer of 2 or more, a plurality of R₁₉'s are the same as or different from each other. In Formula 3-11, if n₂₀ is an integer of 2 or more, a plurality of R₂₀'s are the same as or different from each other.

Considering the structure of the nitrogen-containing compound represented by Formula 1 again, the nitrogen-containing compound represented by Formula 1 may be represented by any one among Formula 1-1 to Formula 1-3 below:

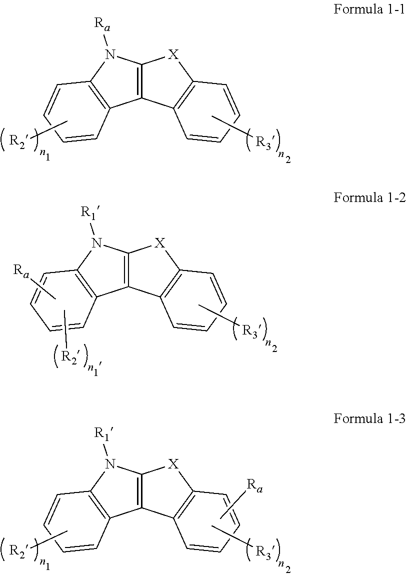

Formula 1-1 to Formula 1-3 are the cases where the position, at which a carbazole substituent represented by Formula 2-1 or Formula 2-2 is linked, is specified. In Formula 1-1 to Formula 1-3, Rₐ is a substituent represented by Formula 2-1 or Formula 2-2 above. That is, Formula 1-1 is the case where R₁ in Formula 1 is a carbazole substituent represented by Formula 2-1 or Formula 2-2, Formula 1-2 is the case where one of R₂'s in Formula 1 is a carbazole substituent represented by Formula 2-1 or Formula 2-2, and Formula 1-3 is the case where one of R₃'s in Formula 1 is a carbazole substituent represented by Formula 2-1 or Formula 2-2.

In Formula 1-1 to Formula 1-3, R₁′, R₂′, and R₃′ are each independently a hydrogen atom, a deuterium atom, a halogen atom, a nitro group, a substituted or unsubstituted silyl group, a substituted or unsubstituted boron group, a substituted or unsubstituted oxy group, a substituted or unsubstituted thio group, a substituted or unsubstituted carbonyl group, a substituted or unsubstituted alkyl group having 1 to 30 carbon atoms, a substituted or unsubstituted aryl group having 6 to 60 ring-forming carbon atoms, or a substituted or unsubstituted heteroaryl group having 2 to 60 ring-forming carbon atoms. For example, R₁′, R₂′, and R₃′ may be each independently a hydrogen atom, a deuterium atom, a substituted or unsubstituted phenyl group, a substituted or unsubstituted biphenyl group, a substituted or unsubstituted terphenyl group, or a substituted or unsubstituted naphthyl group. The case where each of R₁′, R₂′, and R₃′ is represented by Formula 2-1 or Formula 2-2 above is excluded.

In Formula 1-2 to Formula 1-3, n₁′ and n₂′ are each independently an integer of 1 to 3. In Formula 1-2, if n₁′ is an integer of 2 or more, a plurality of R₂'s may be the same as or different from each other. In Formula 1-3, if n₂′ is an integer of 2 or more, a plurality of R₃'s may be the same as or different from each other.

The nitrogen-containing compound may be any one among compounds represented by Compound Group 1 to Compound Group 3 below. The light emitting device ED may include at least one nitrogen-containing compound among the compounds represented by Compound Group 1 to Compound Group 3 in the hole transport region HTR.

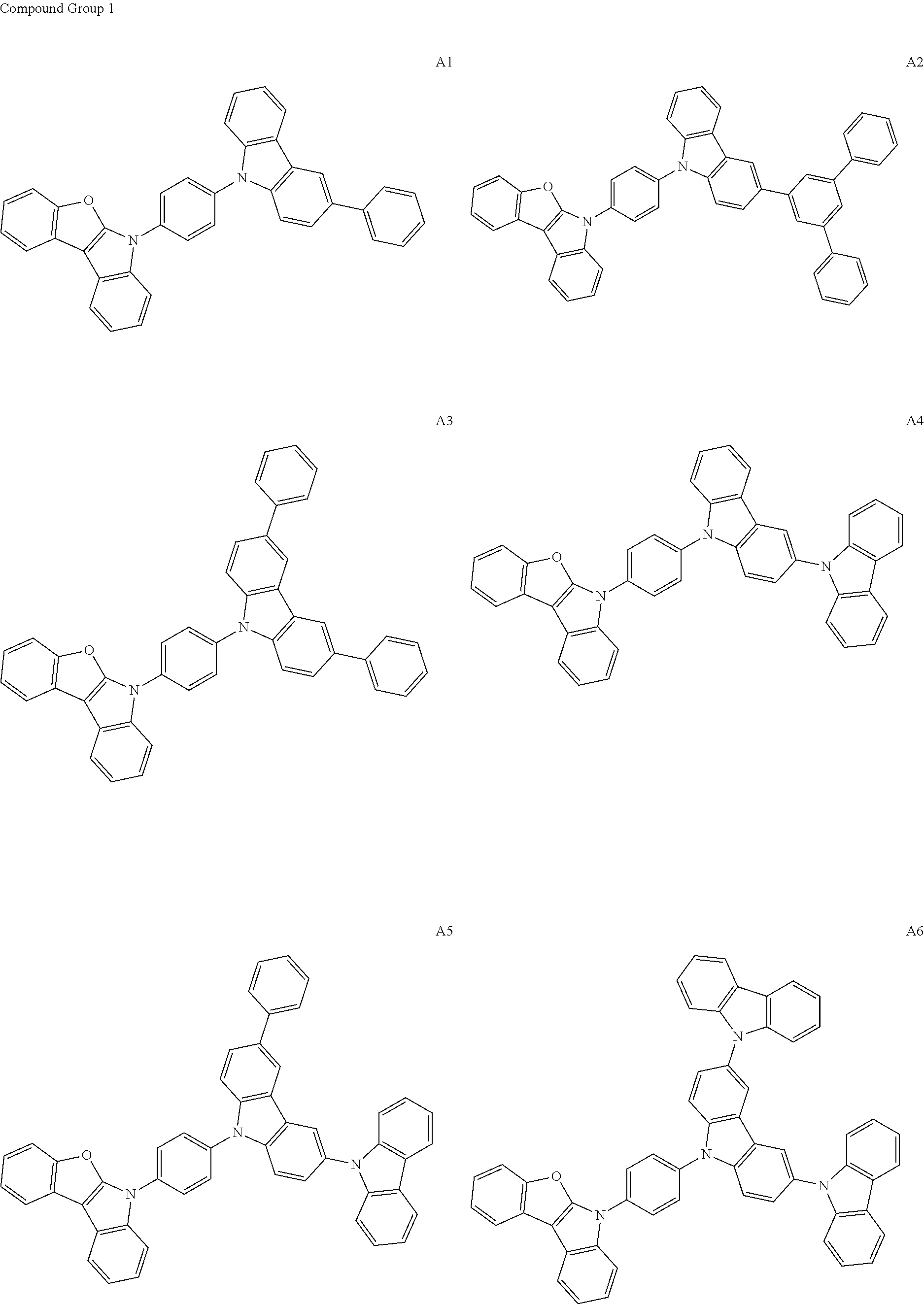

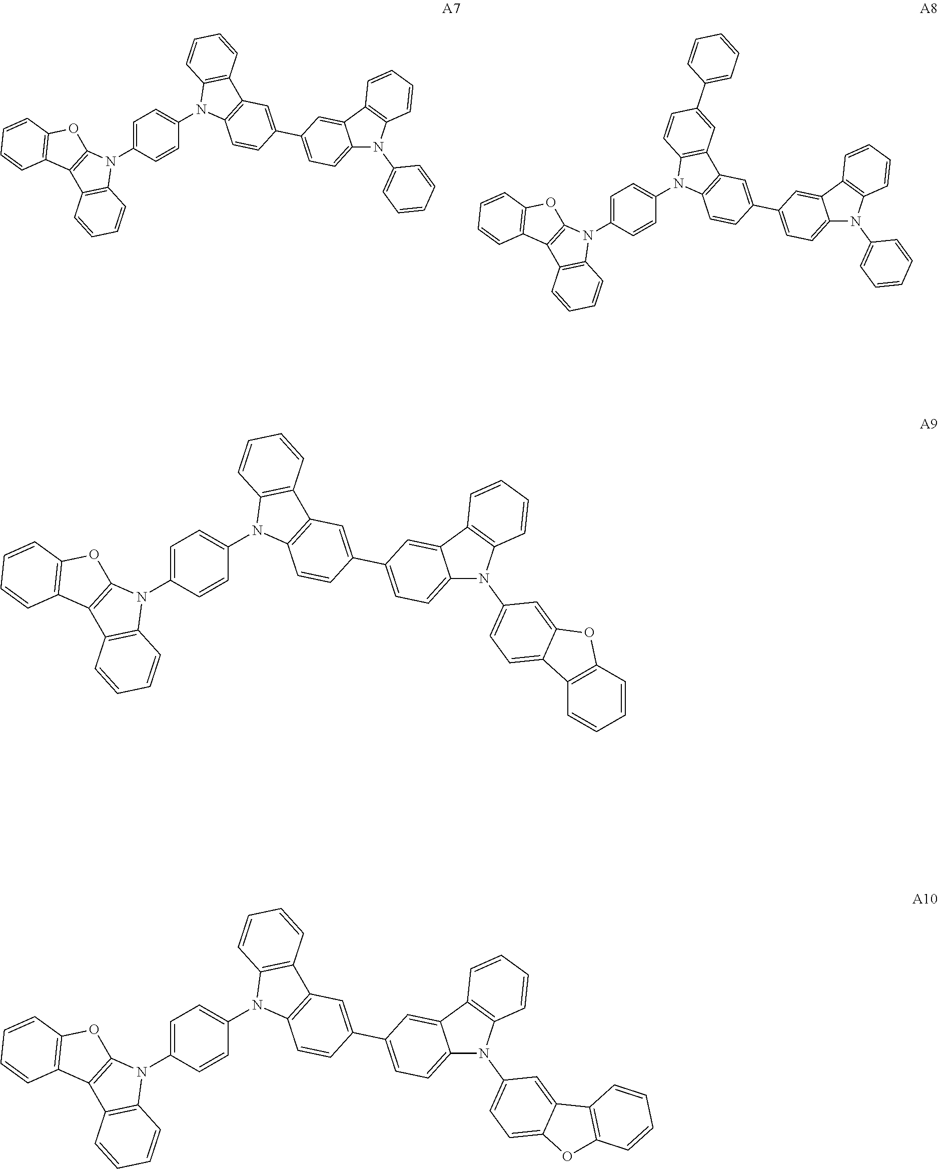 [SMILES: c1ccc(-n2c3ccccc3c3cc(-c4ccc5c(c4)c4ccccc4n5-c4ccc(-n5c6ccccc6c6c7ccccc7oc65)cc4)ccc32)cc1 ; c1ccc(-c2ccc3c(c2)c2cc(-c4ccc5c(c4)c4ccccc4n5-c4ccccc4)ccc2n3-c2ccc(-n3c4ccccc4c4c5ccccc5oc43)cc2)cc1 ; c1ccc2c(c1)oc1cc(-n3c4ccccc4c4cc(-c5ccc6c(c5)c5ccccc5n6-c5ccc(-n6c7ccccc7c7c8ccccc8oc76)cc5)ccc43)ccc12 ; c1ccc2c(c1)oc1ccc(-n3c4ccccc4c4cc(-c5ccc6c(c5)c5ccccc5n6-c5ccc(-n6c7ccccc7c7c8ccccc8oc76)cc5)ccc43)cc12]

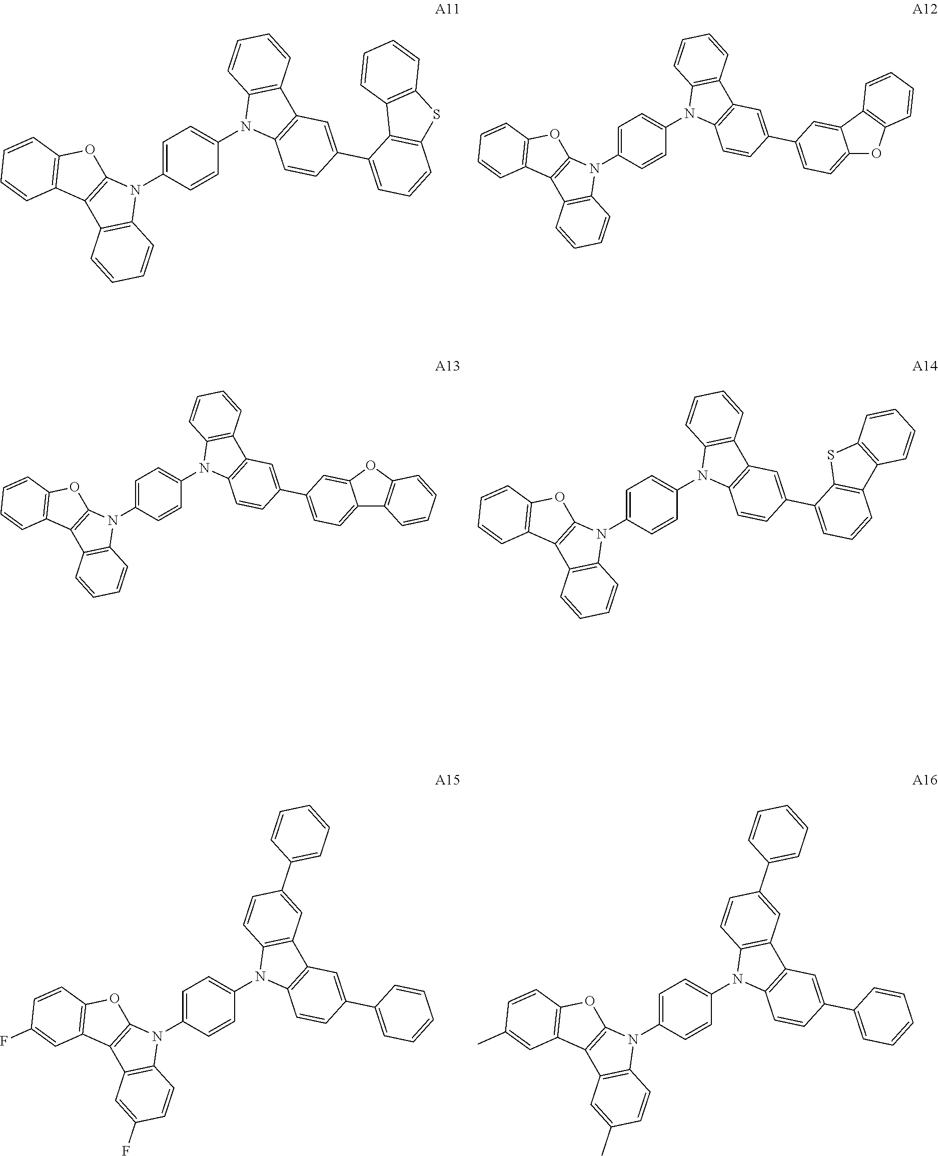

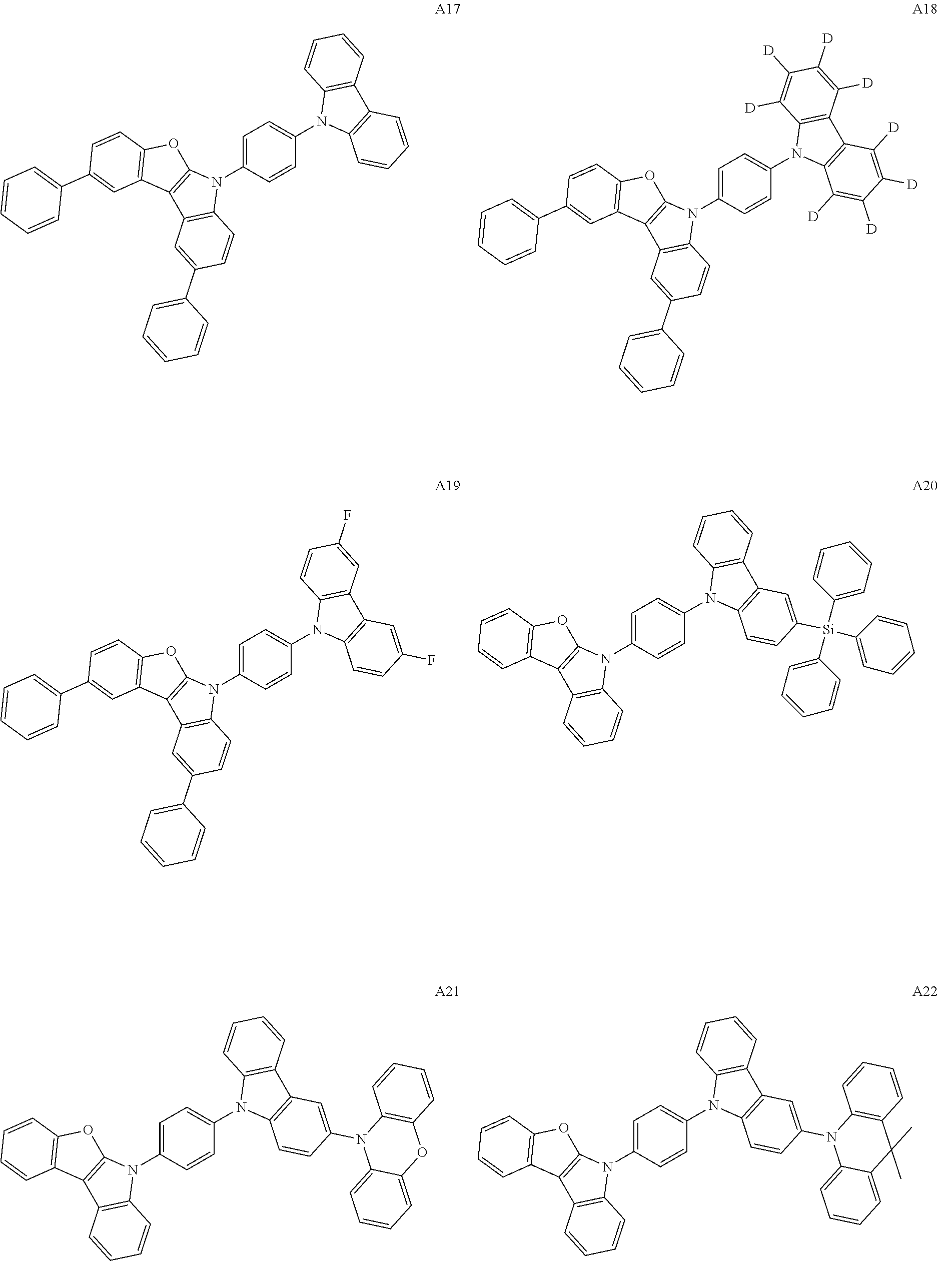

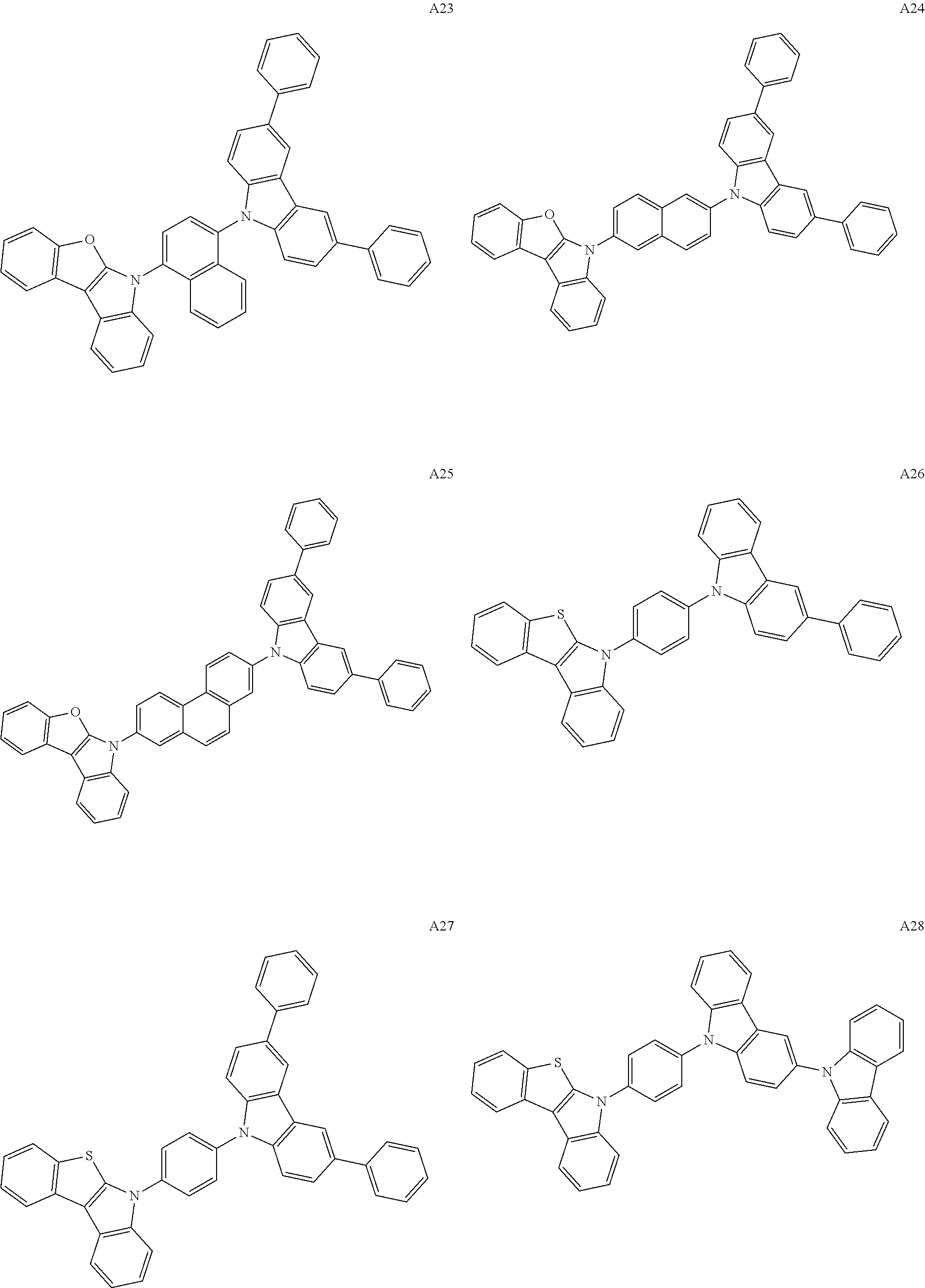

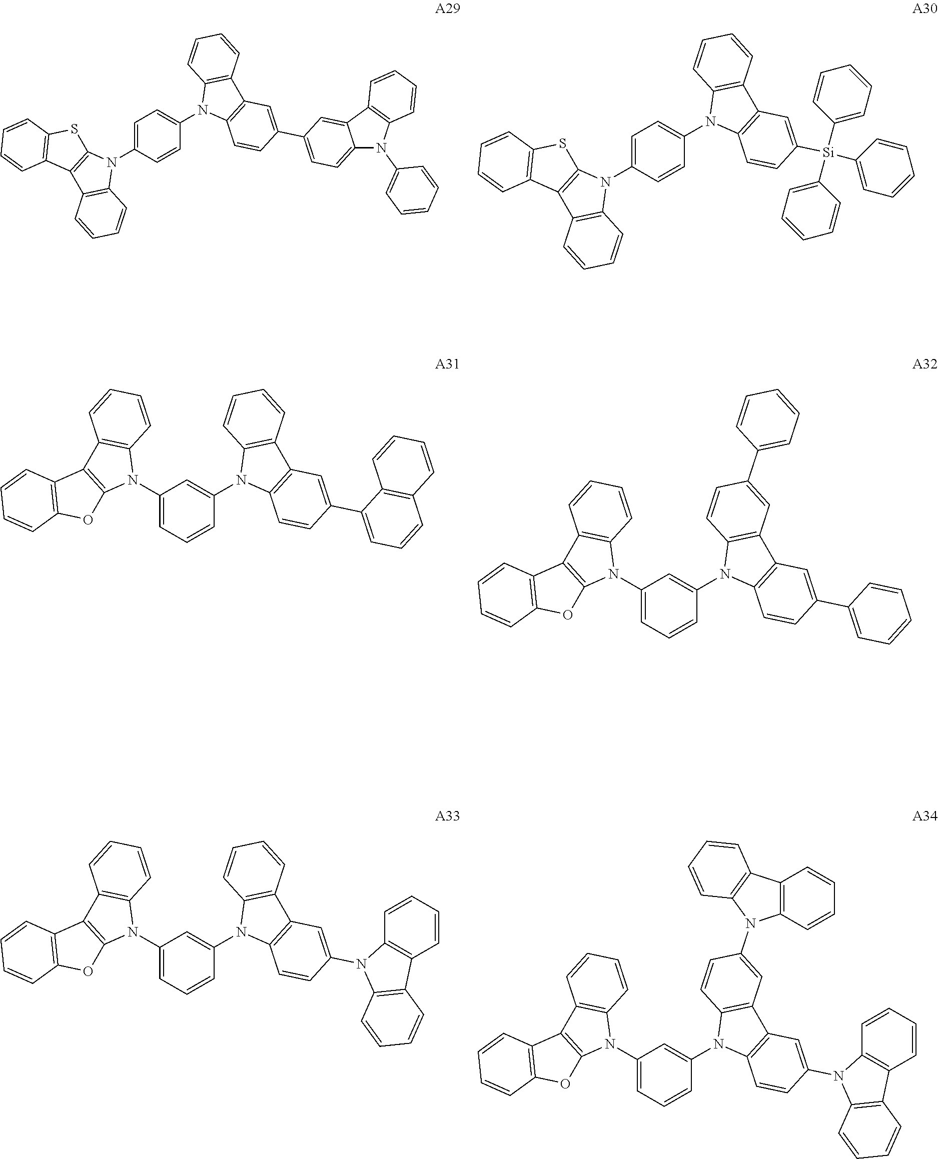

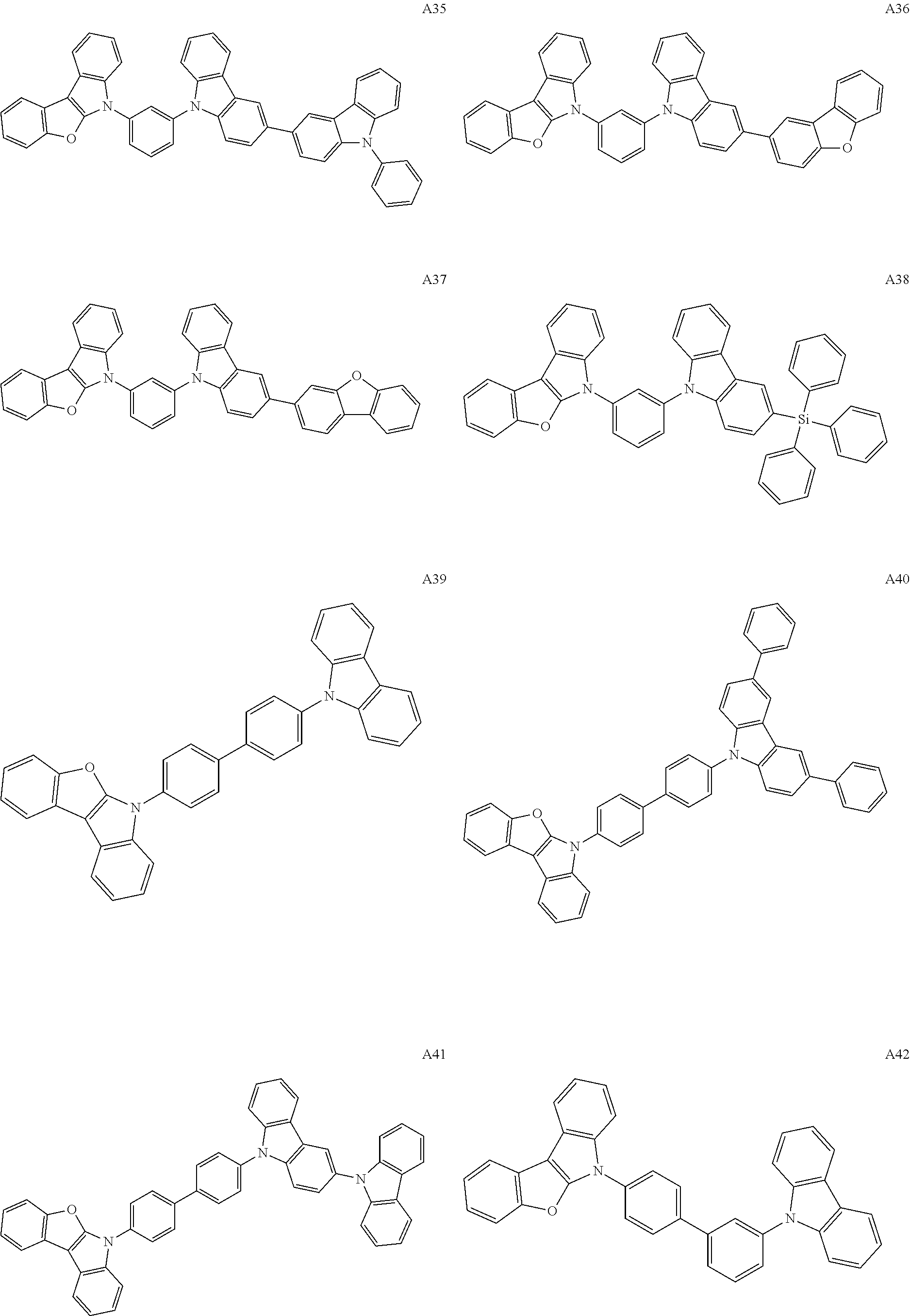

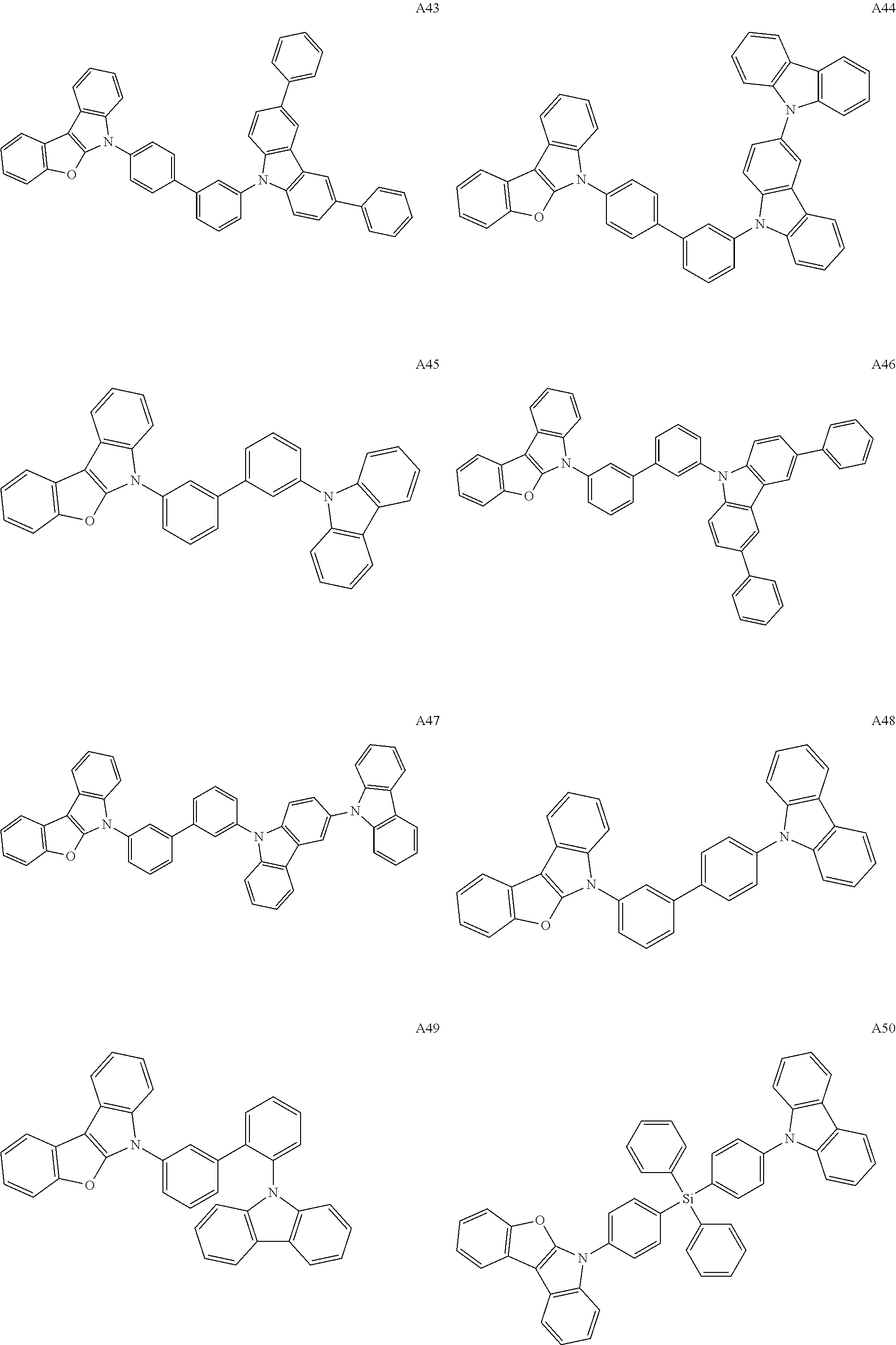

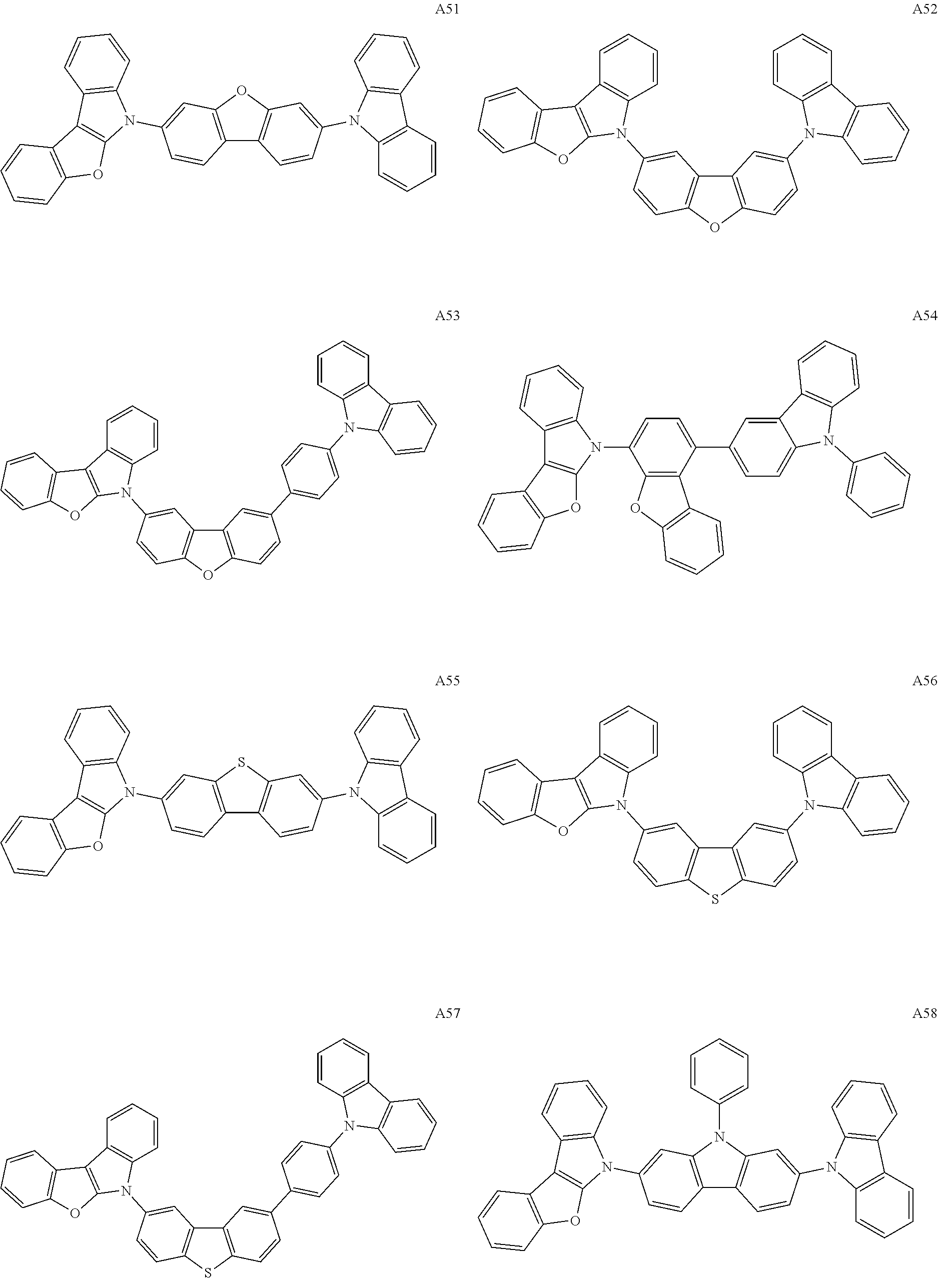

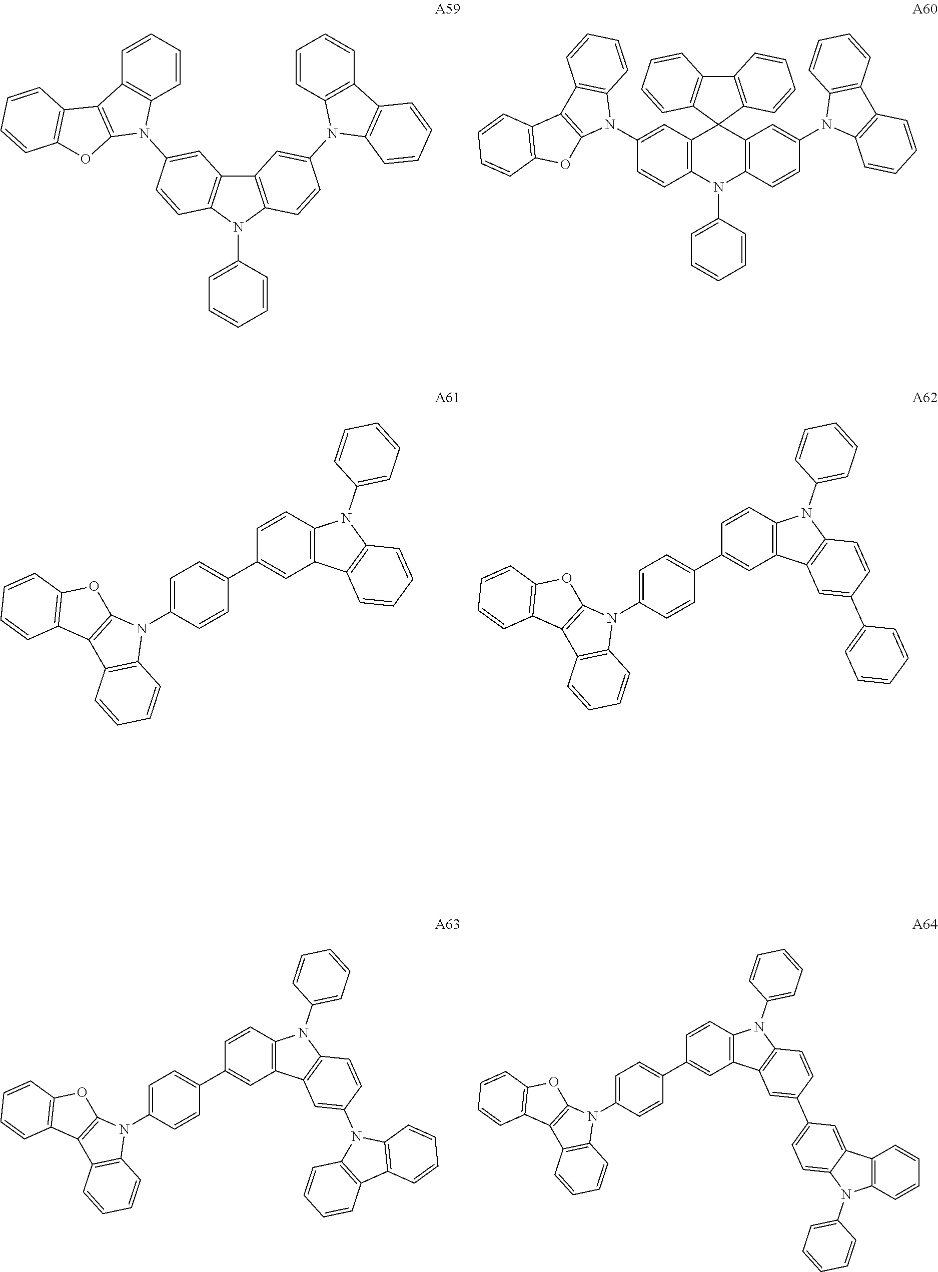

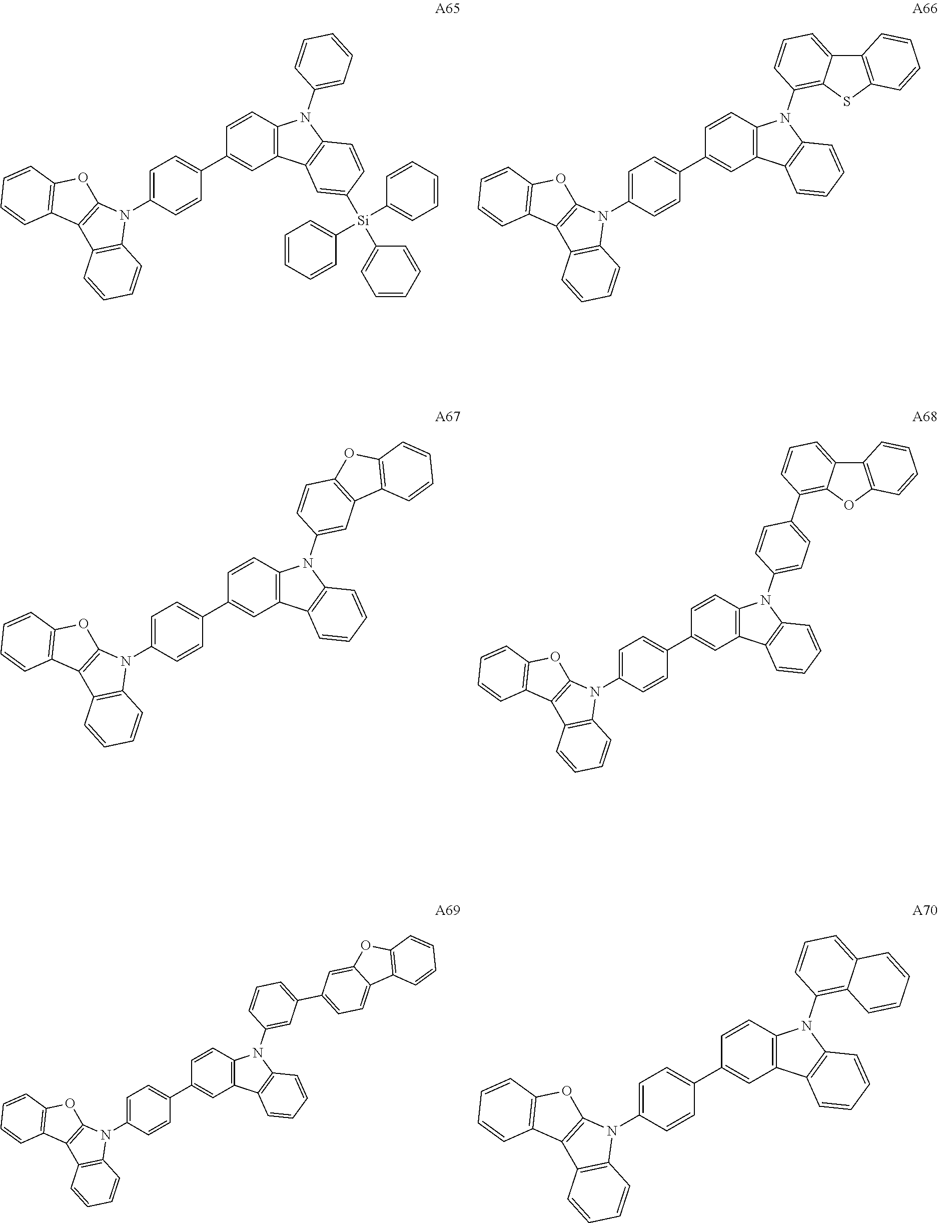

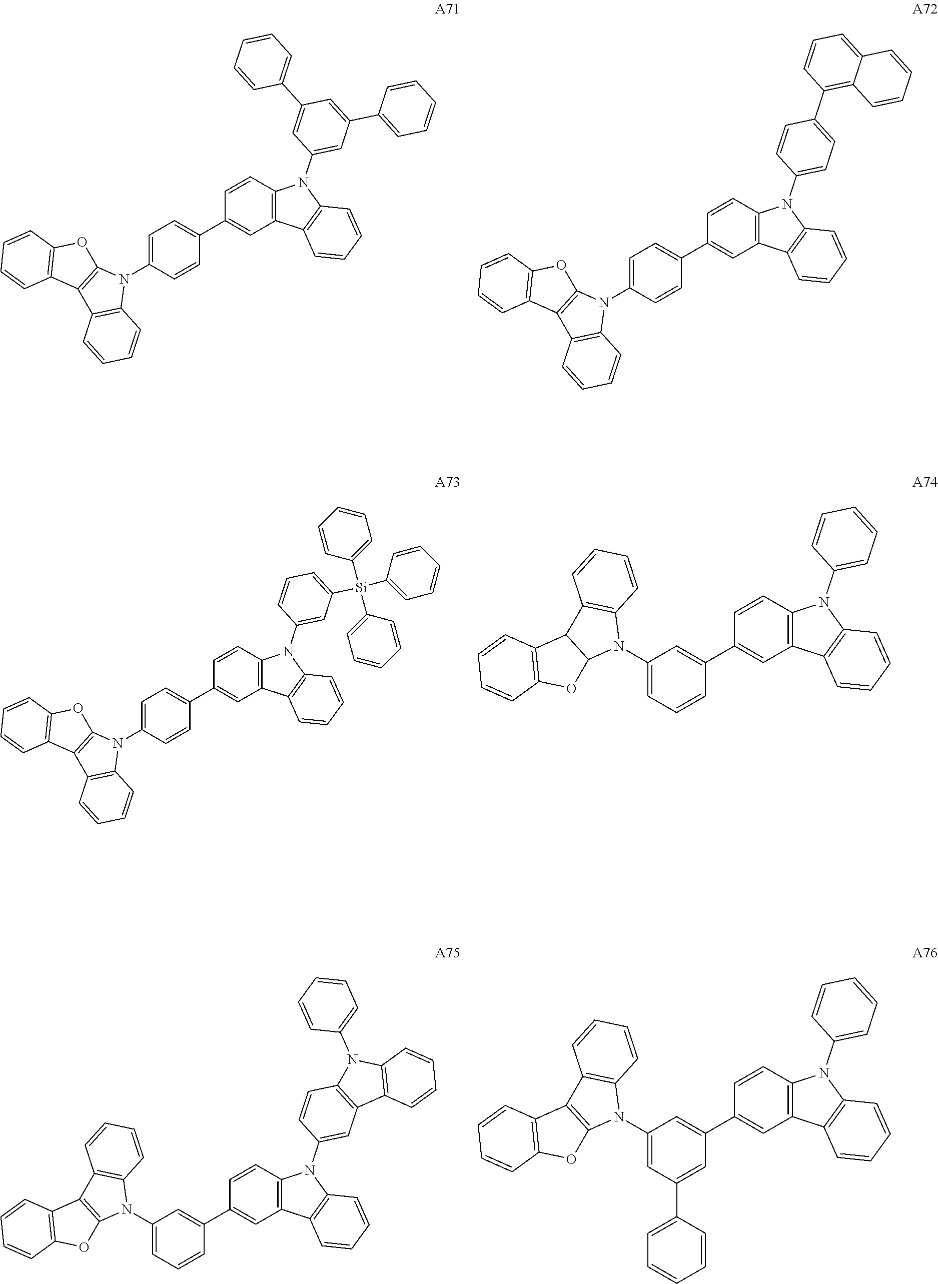

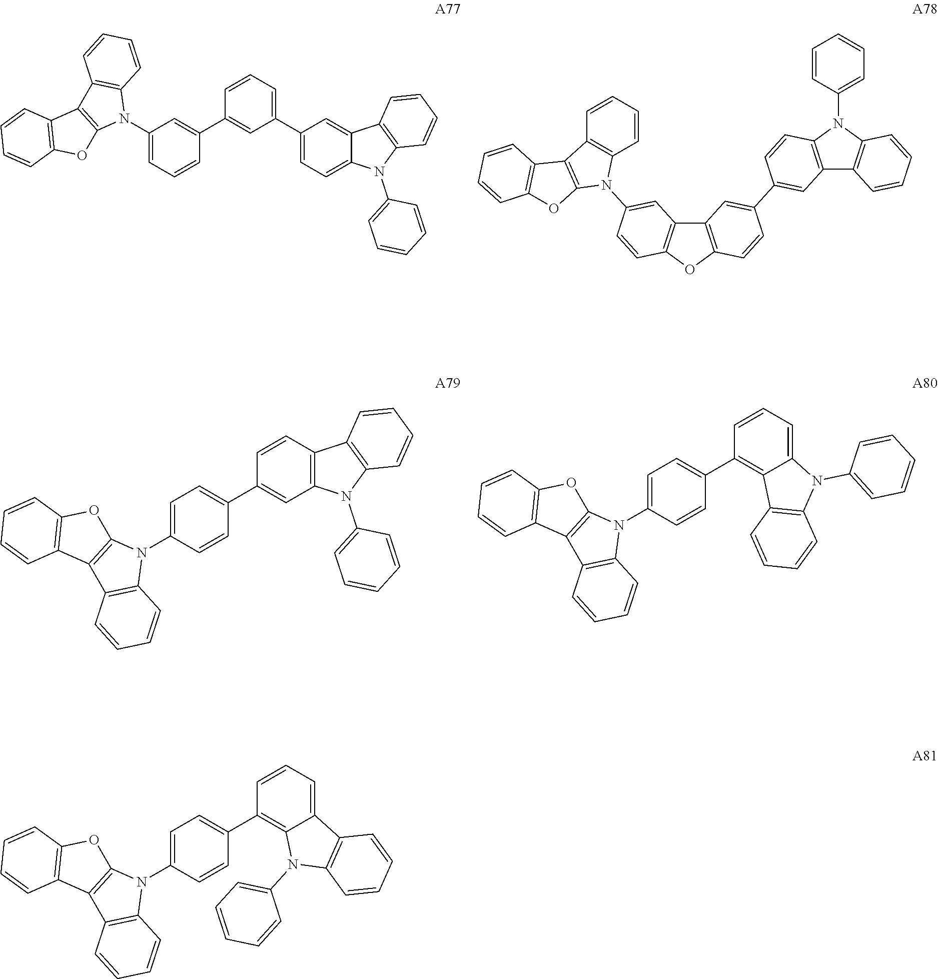 [SMILES: c1ccc(-n2c3ccccc3c3cc(-c4cccc(-c5cccc(-n6c7ccccc7c7c8ccccc8oc76)c5)c4)ccc32)cc1 ; c1ccc(-n2c3ccccc3c3cc(-c4ccc5oc6ccc(-n7c8ccccc8c8c9ccccc9oc87)cc6c5c4)ccc32)cc1 ; c1ccc(-n2c3ccccc3c3ccc(-c4ccc(-n5c6ccccc6c6c7ccccc7oc65)cc4)cc32)cc1 ; c1ccc(-n2c3ccccc3c3c(-c4ccc(-n5c6ccccc6c6c7ccccc7oc65)cc4)cccc32)cc1 ; c1ccc(-n2c3ccccc3c3cccc(-c4ccc(-n5c6ccccc6c6c7ccccc7oc65)cc4)c32)cc1]

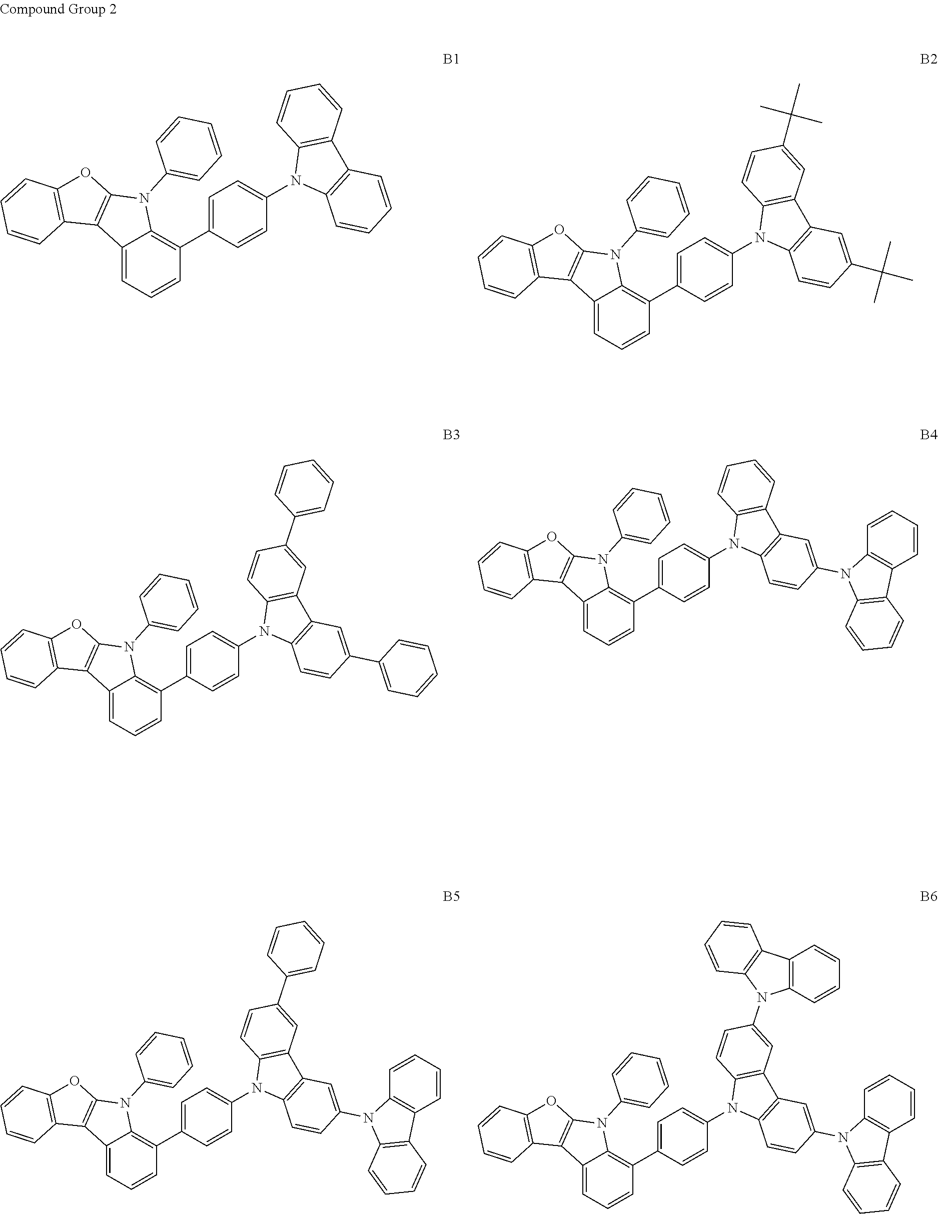

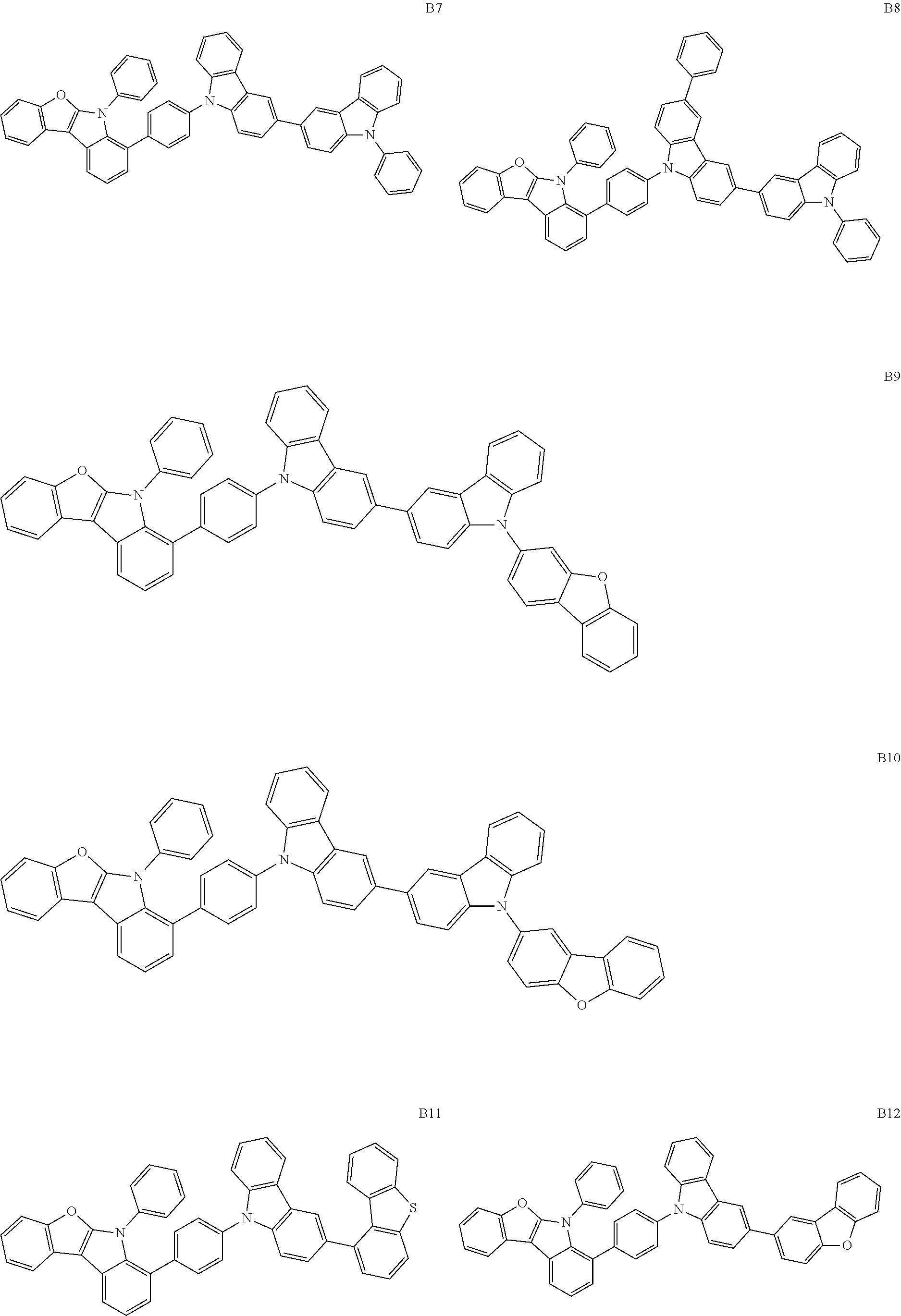

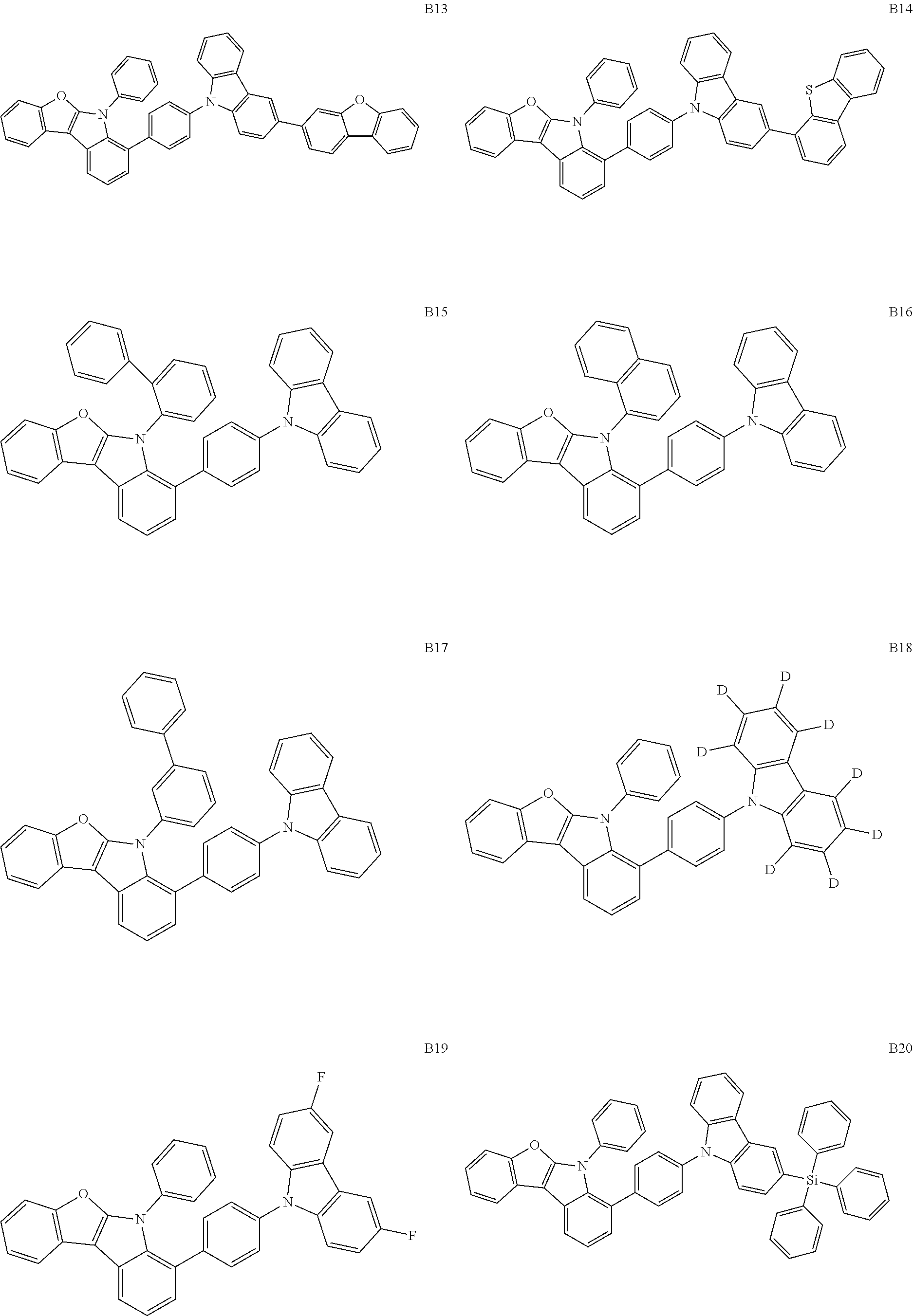

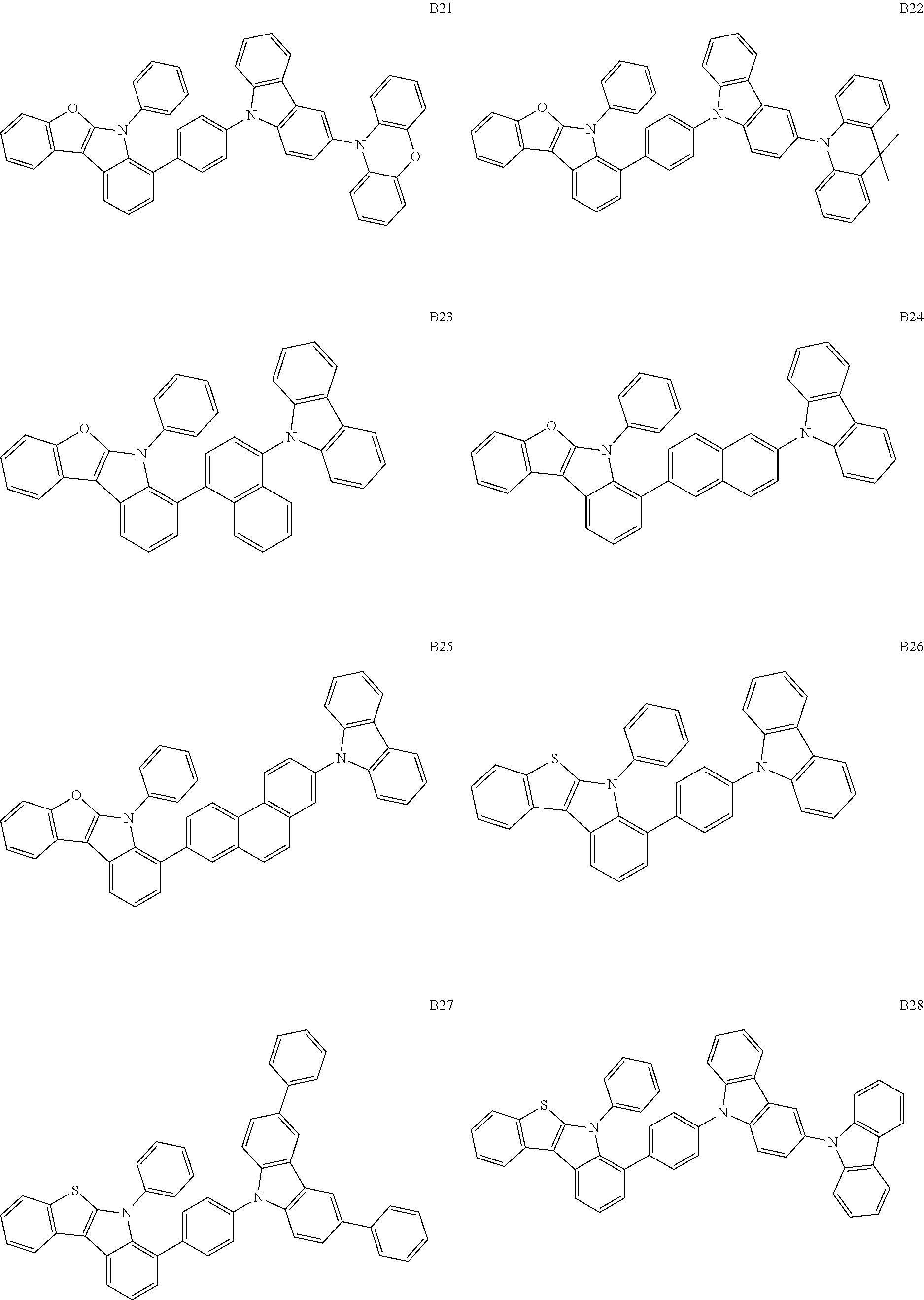

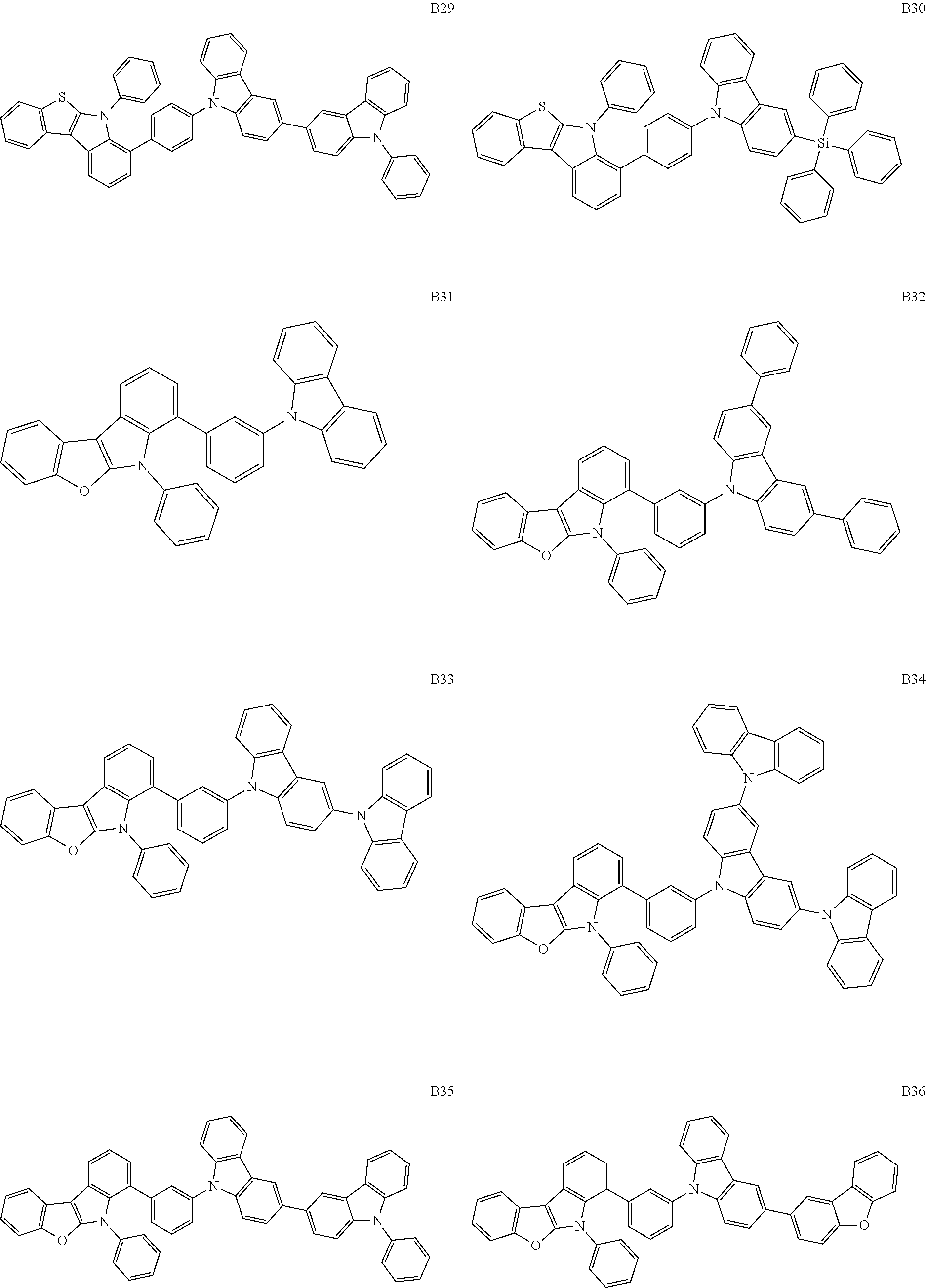

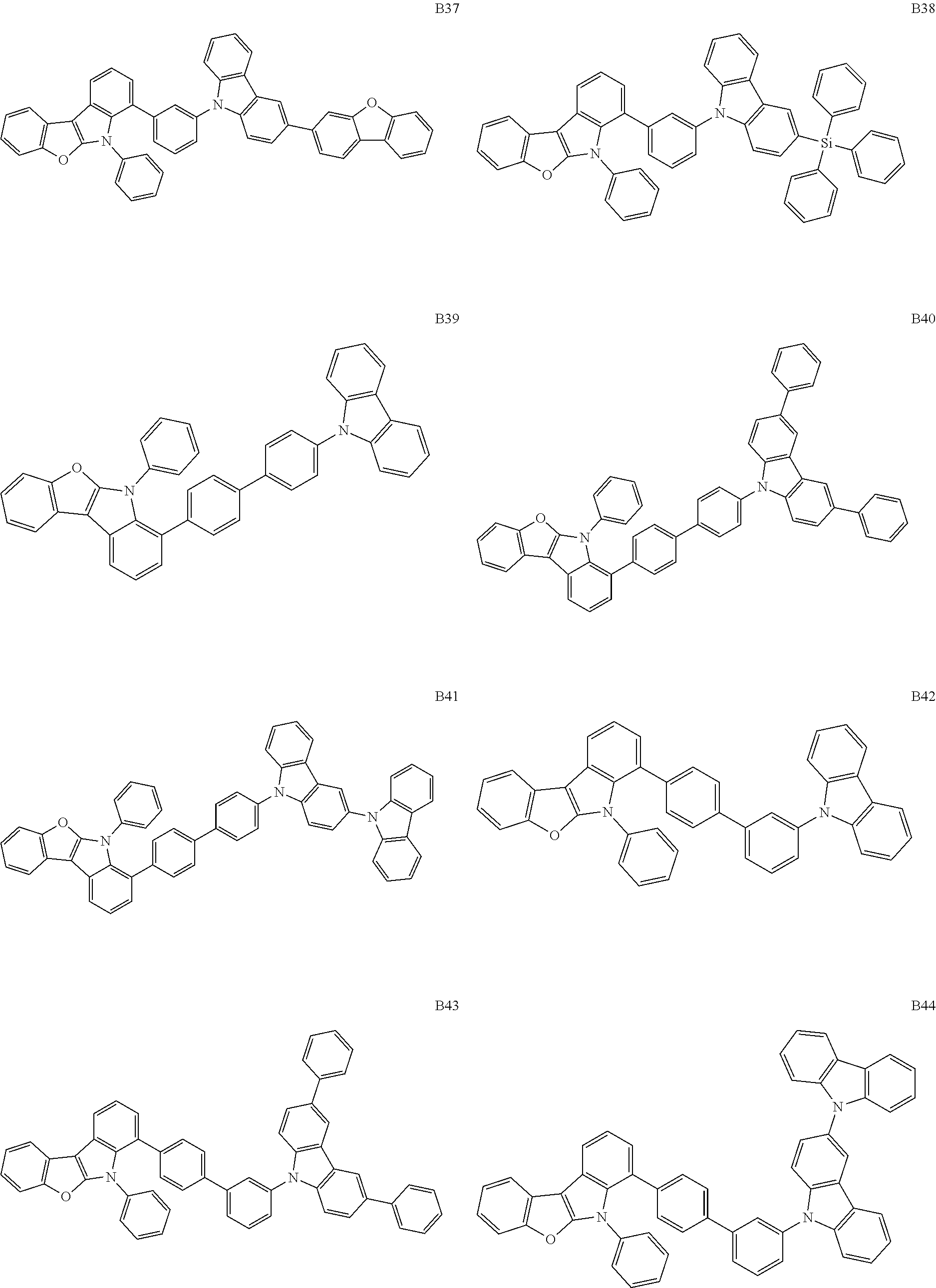

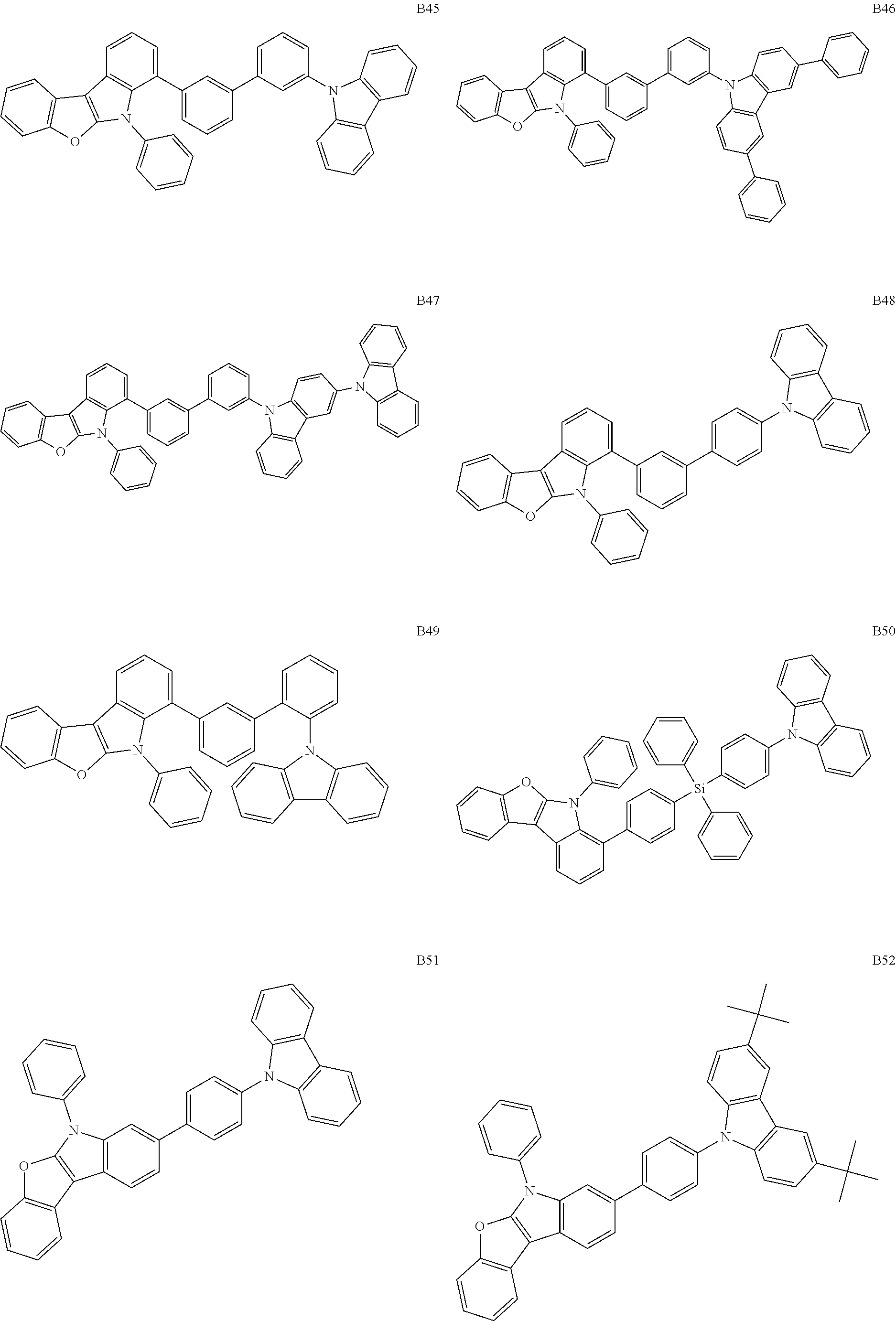

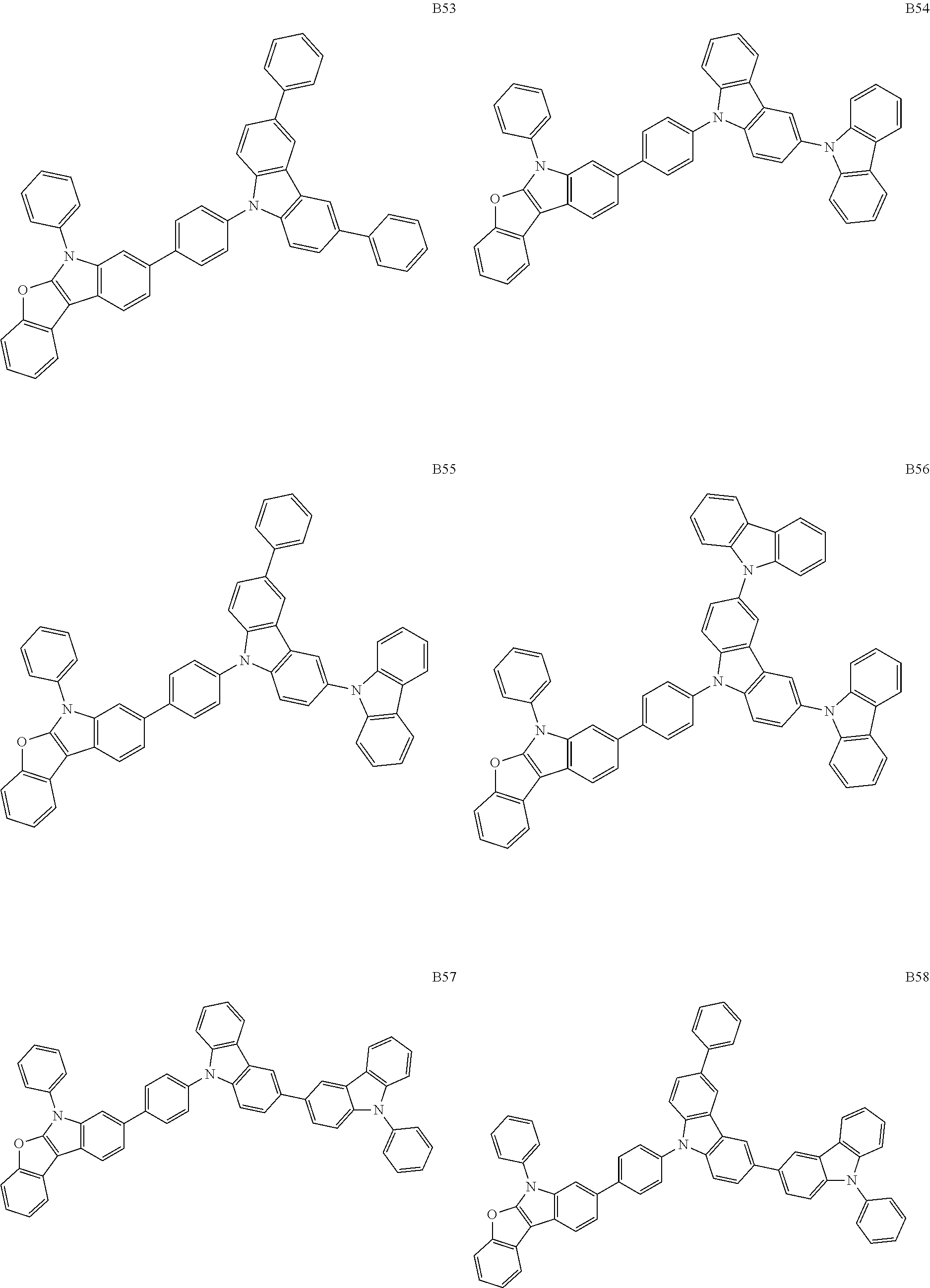

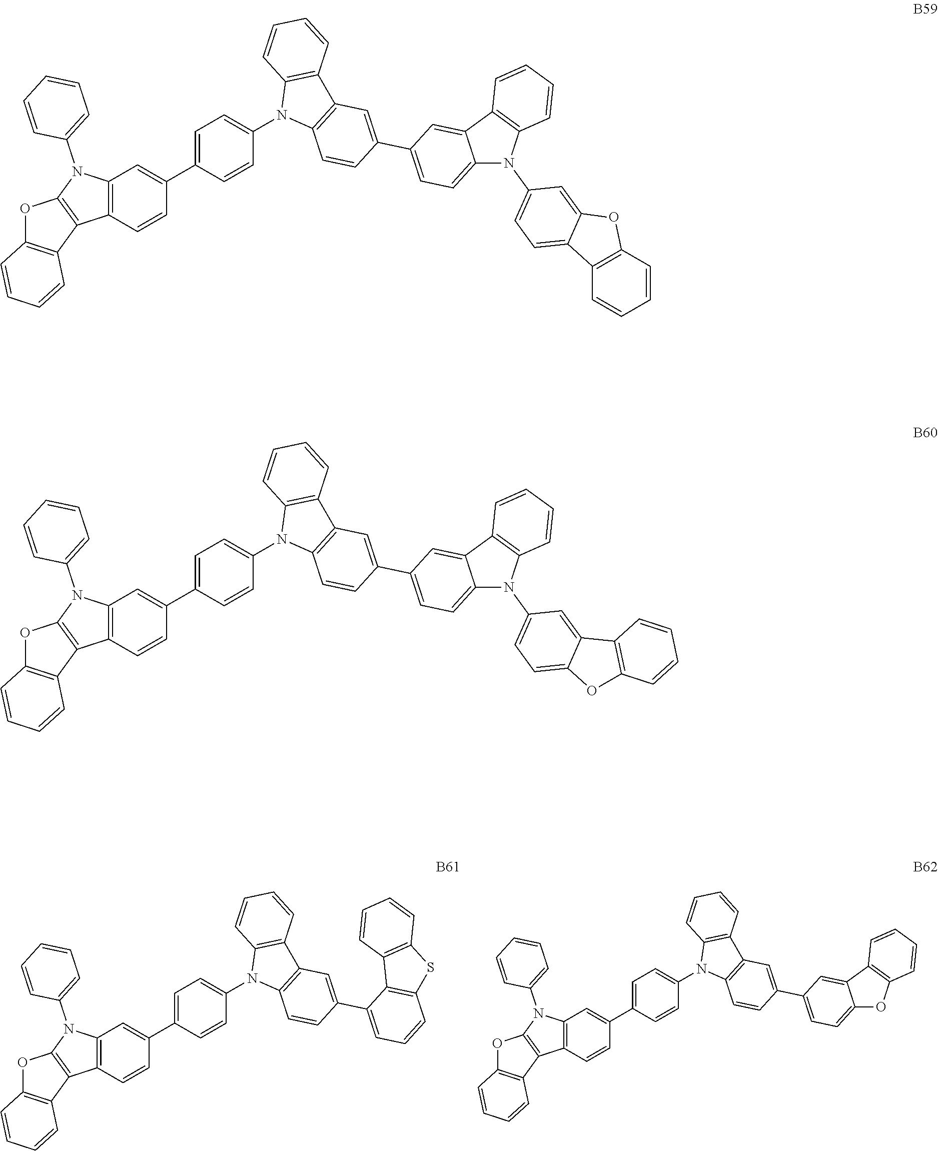 [SMILES: c1ccc(-n2c3cc(-c4ccc(-n5c6ccccc6c6cc(-c7ccc8c(c7)c7ccccc7n8-c7ccc8c(c7)oc7ccccc78)ccc65)cc4)ccc3c3c4ccccc4oc32)cc1 ; c1ccc(-n2c3cc(-c4ccc(-n5c6ccccc6c6cc(-c7ccc8c(c7)c7ccccc7n8-c7ccc8oc9ccccc9c8c7)ccc65)cc4)ccc3c3c4ccccc4oc32)cc1 ; c1ccc(-n2c3cc(-c4ccc(-n5c6ccccc6c6cc(-c7cccc8sc9ccccc9c78)ccc65)cc4)ccc3c3c4ccccc4oc32)cc1 ; c1ccc(-n2c3cc(-c4ccc(-n5c6ccccc6c6cc(-c7ccc8oc9ccccc9c8c7)ccc65)cc4)ccc3c3c4ccccc4oc32)cc1]

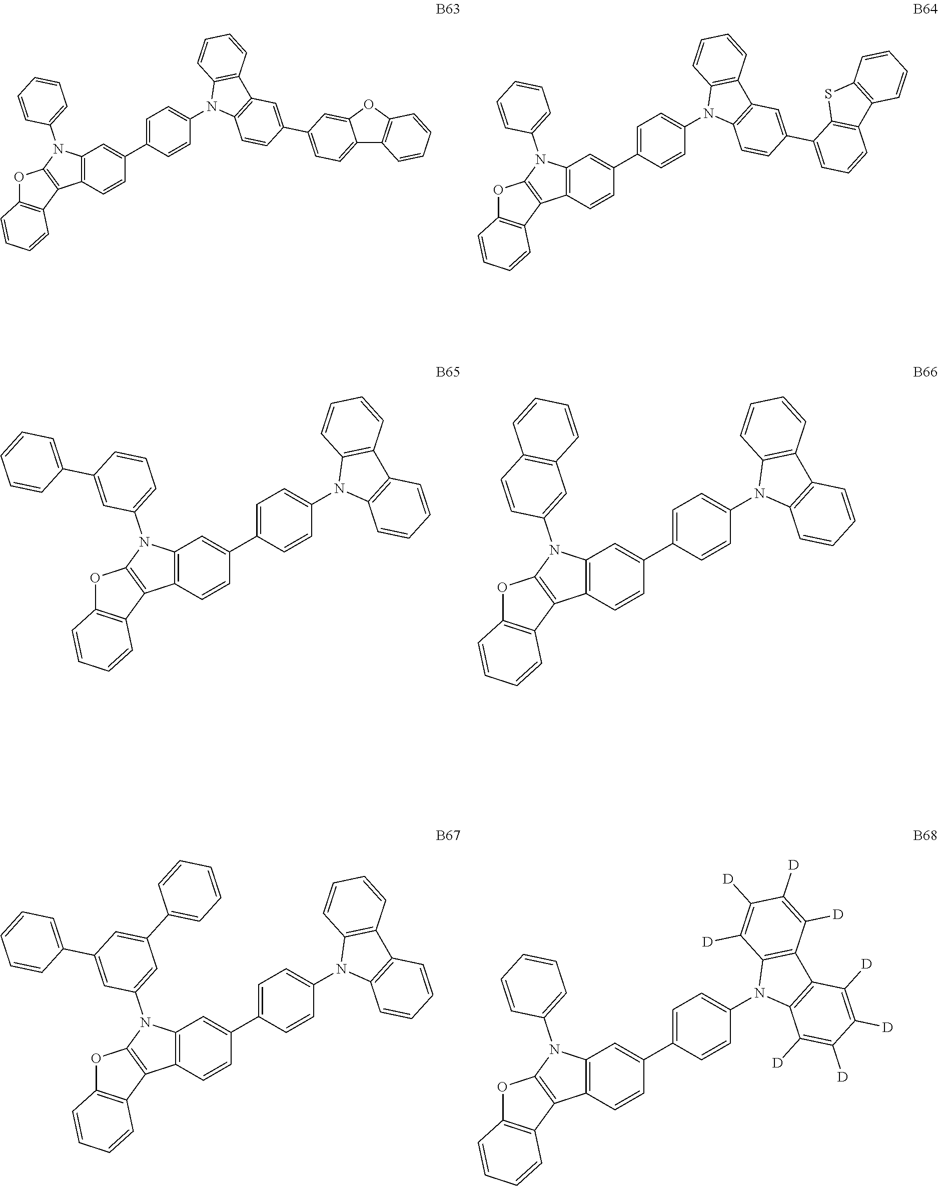

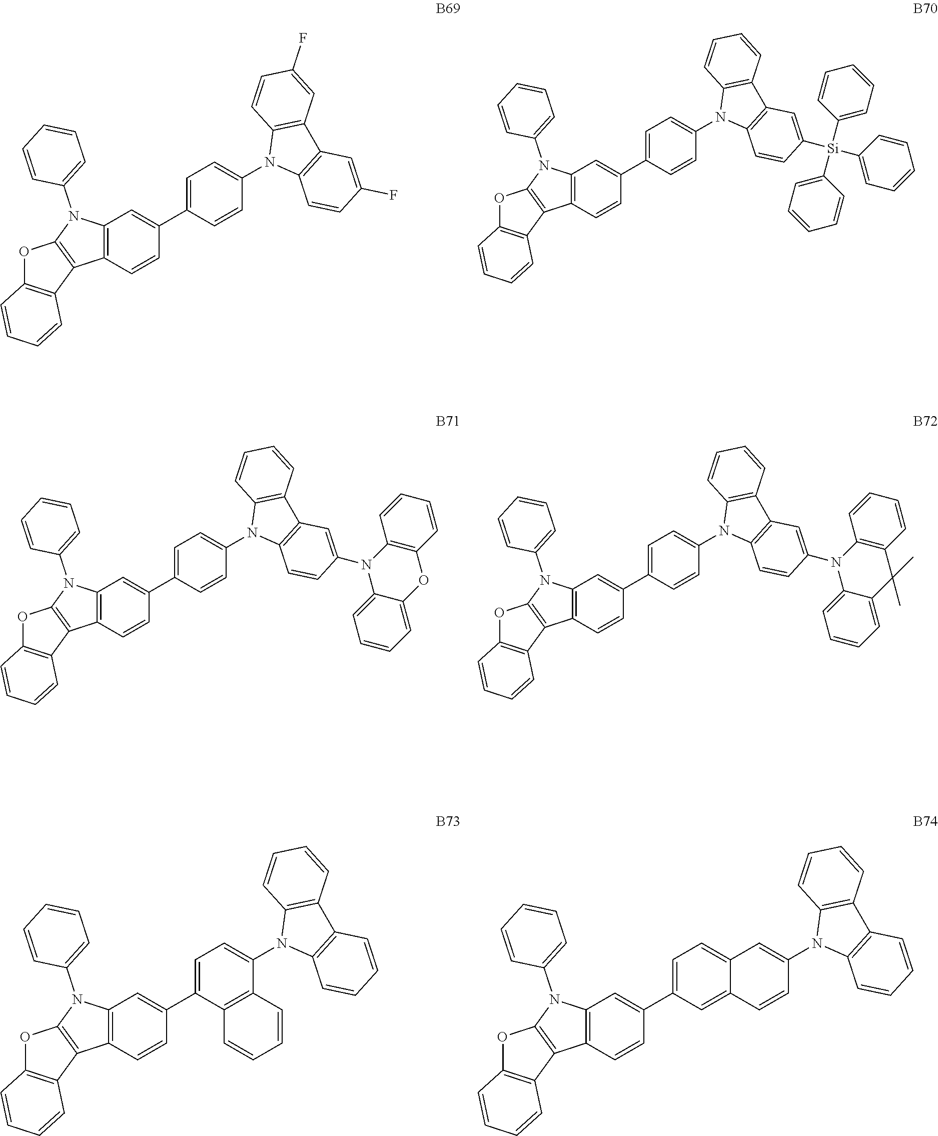

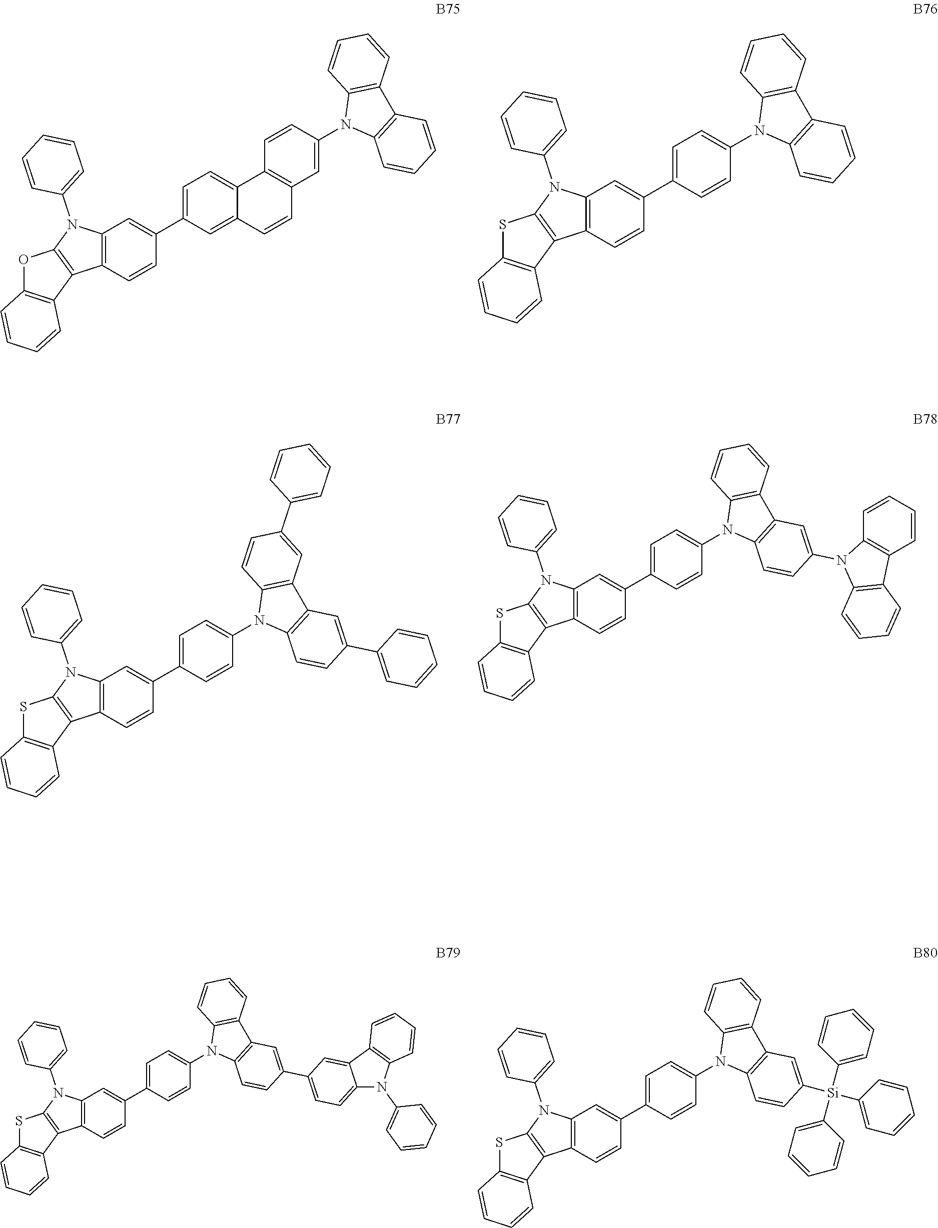

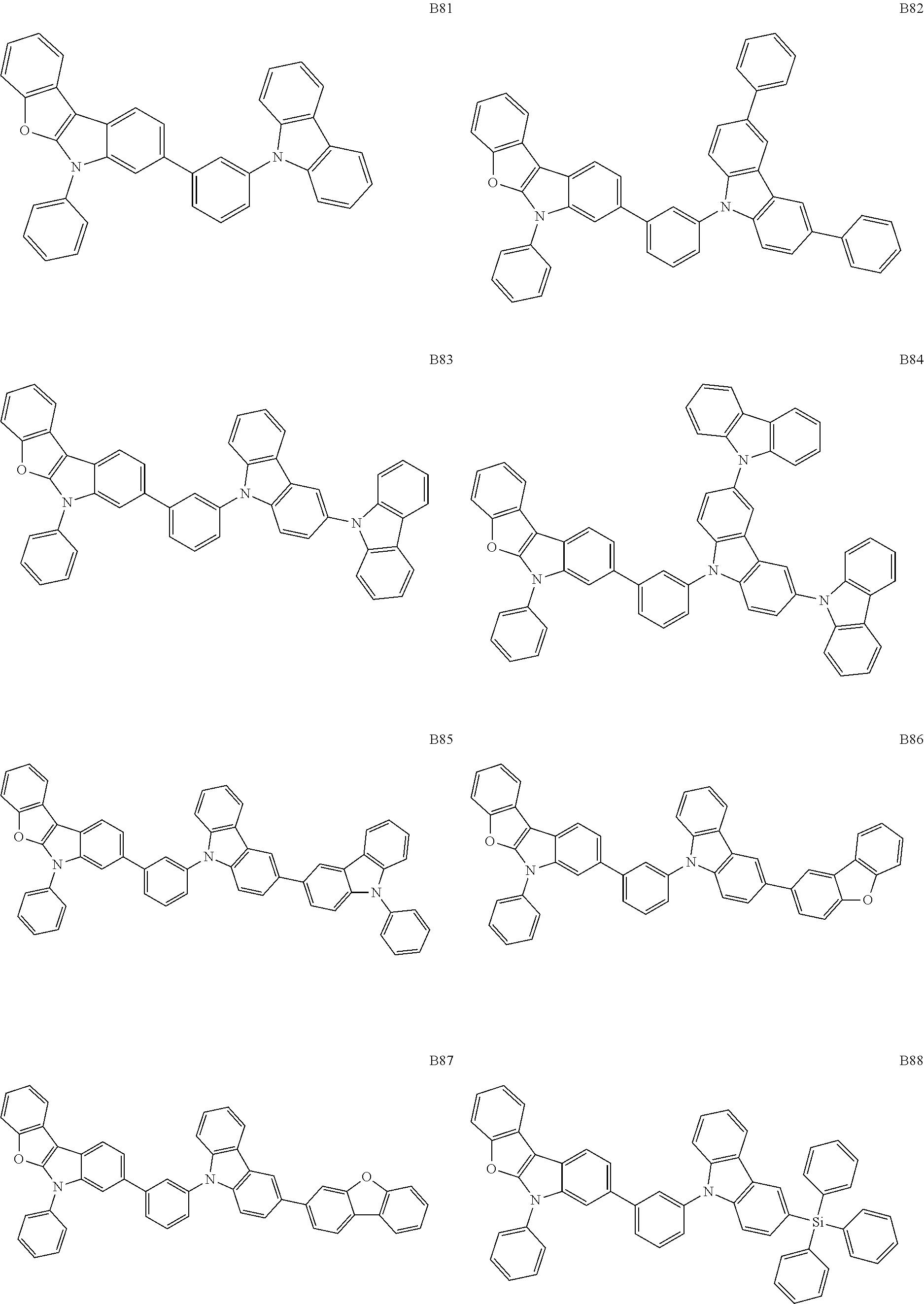

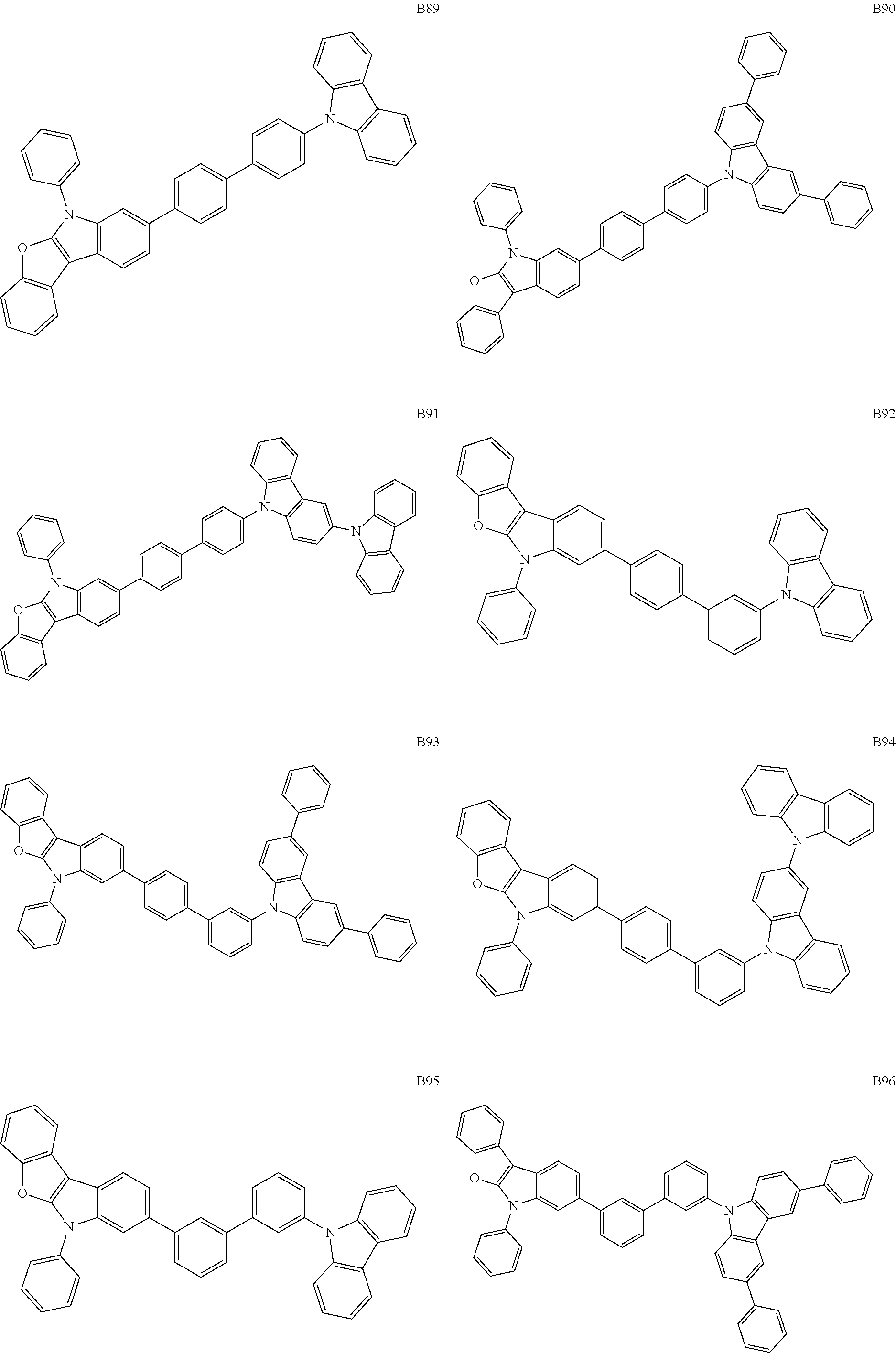

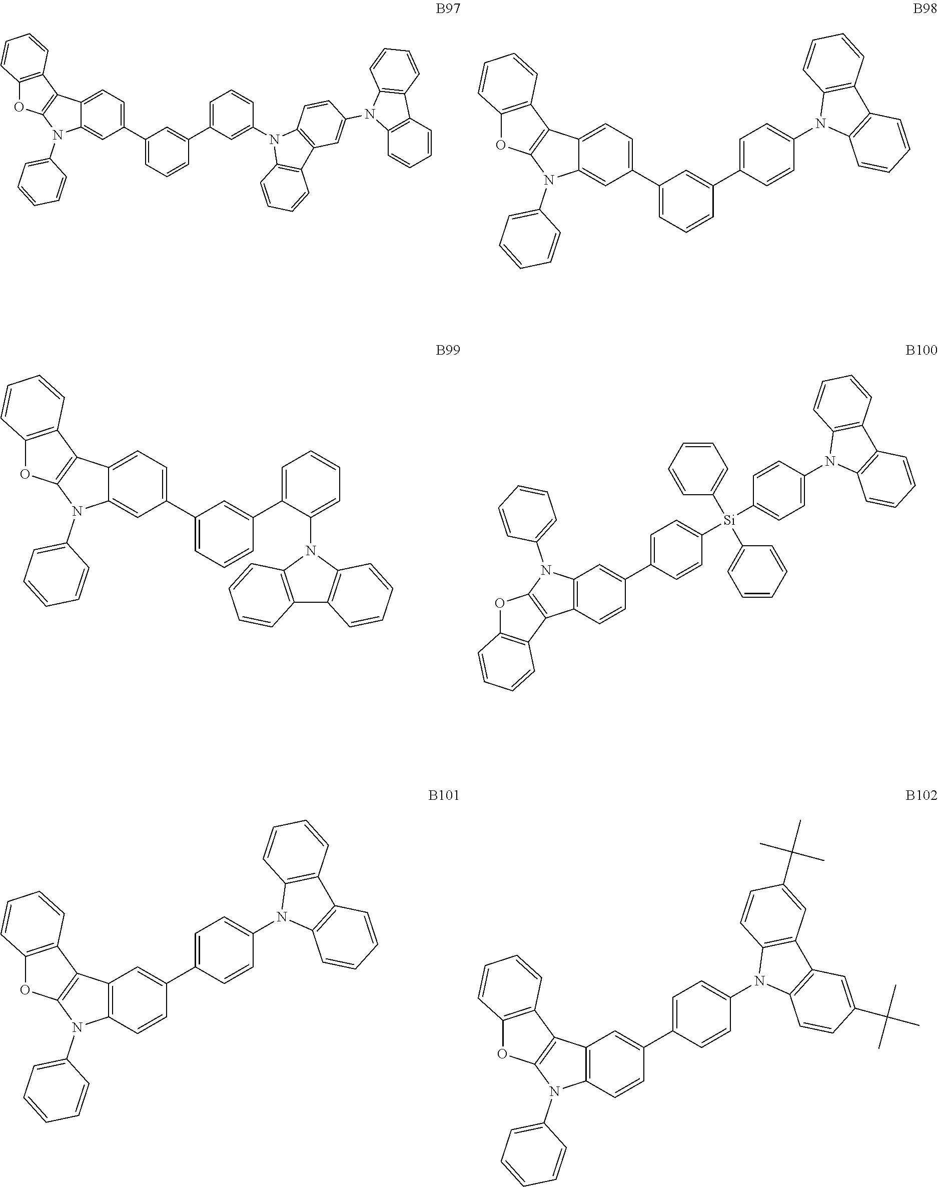

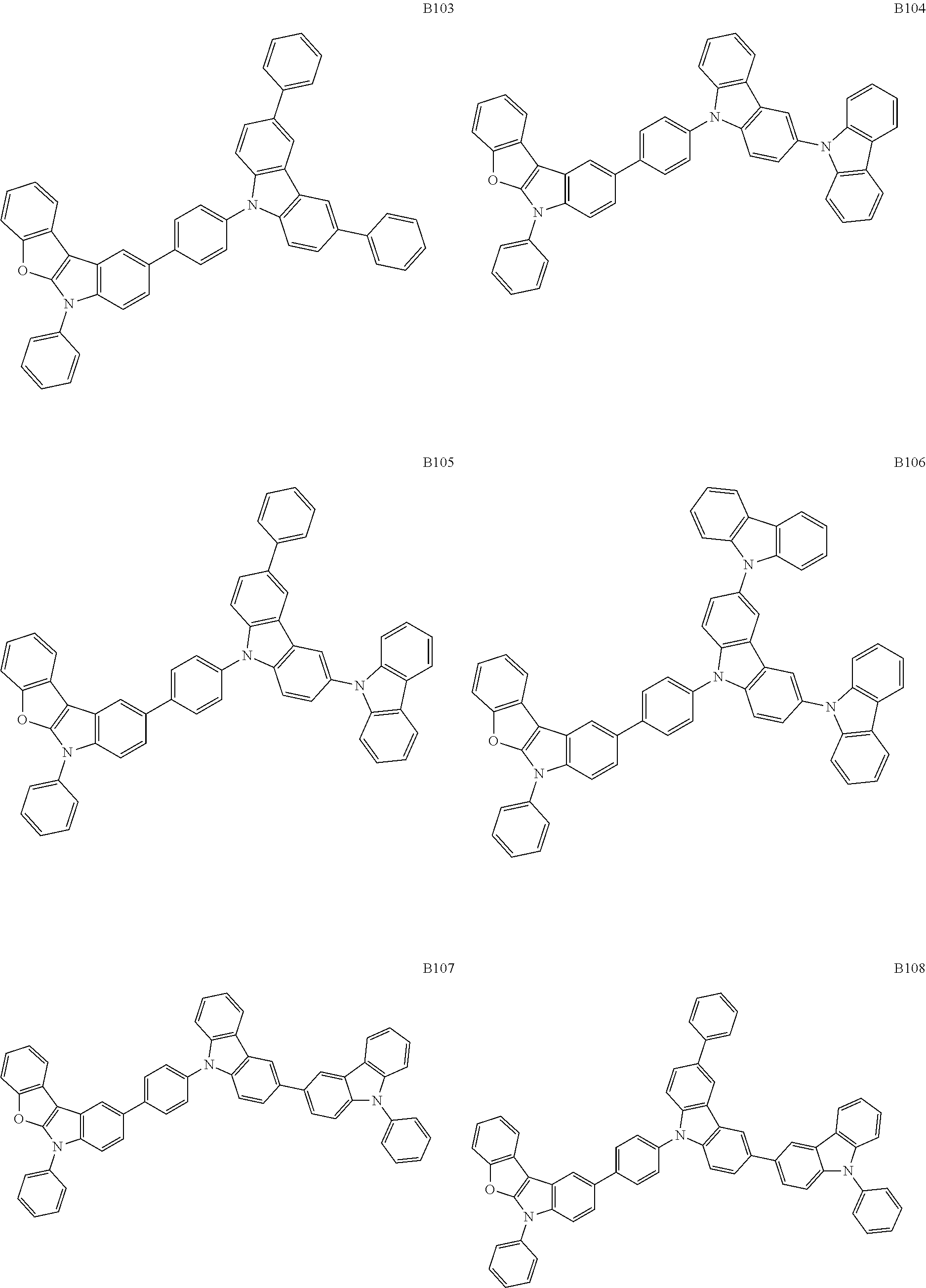

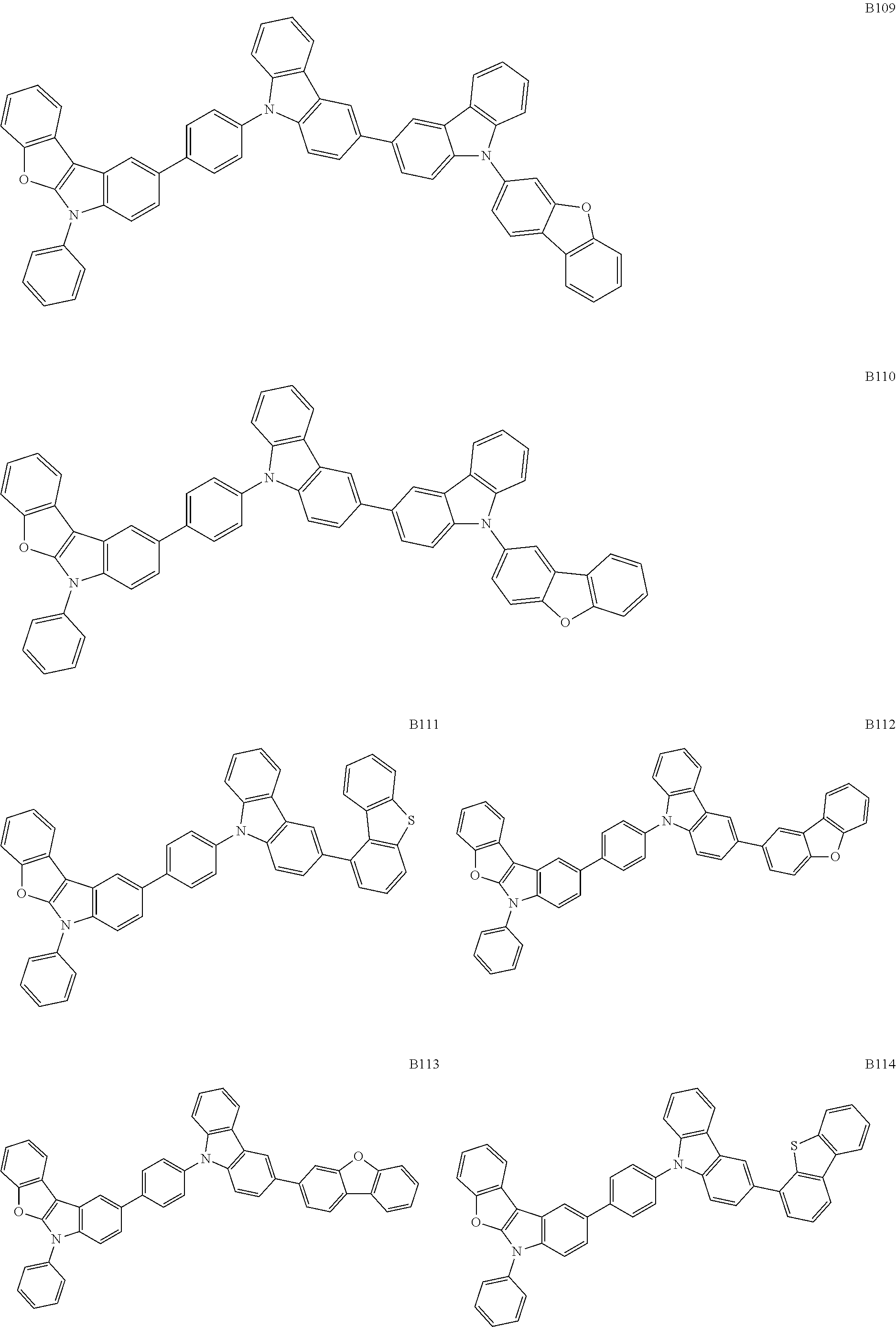

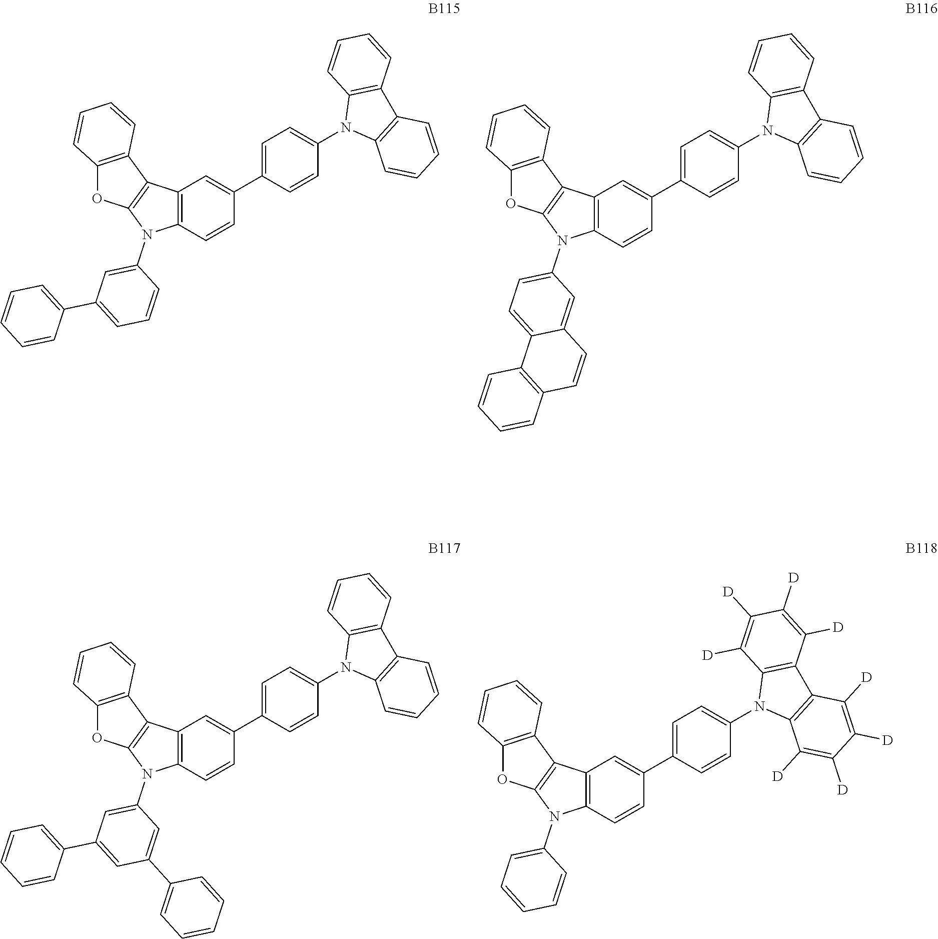 [SMILES: c1ccc(-c2cccc(-n3c4ccc(-c5ccc(-n6c7ccccc7c7ccccc76)cc5)cc4c4c5ccccc5oc43)c2)cc1 ; c1ccc2c(c1)ccc1cc(-n3c4ccc(-c5ccc(-n6c7ccccc7c7ccccc76)cc5)cc4c4c5ccccc5oc43)ccc12 ; c1ccc(-c2cc(-c3ccccc3)cc(-n3c4ccc(-c5ccc(-n6c7ccccc7c7ccccc76)cc5)cc4c4c5ccccc5oc43)c2)cc1 ; [2H]c1c([2H])c([2H])c2c(c1[2H])c1c([2H])c([2H])c([2H])c([2H])c1n2-c1ccc(-c2ccc3c(c2)c2c4ccccc4oc2n3-c2ccccc2)cc1]

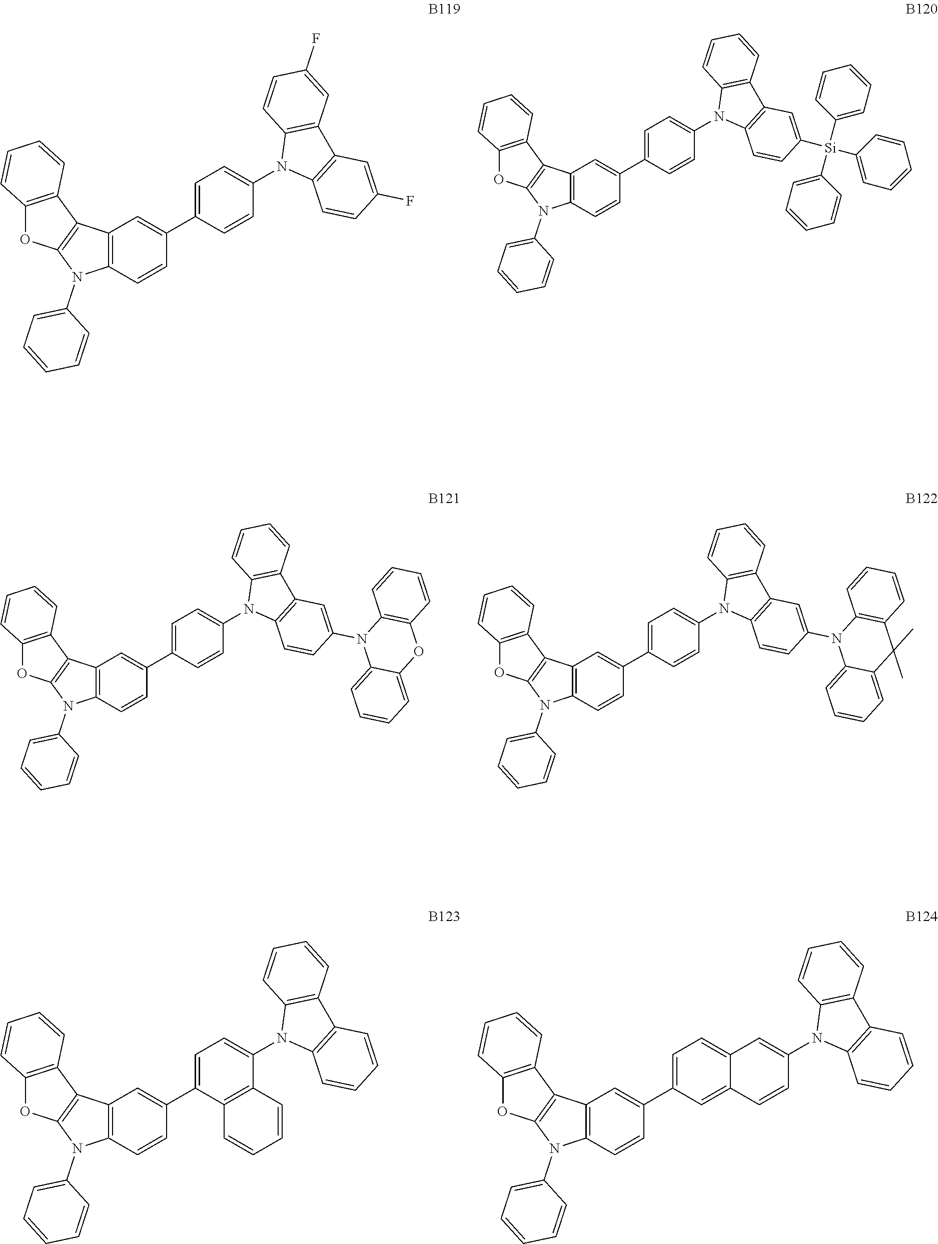

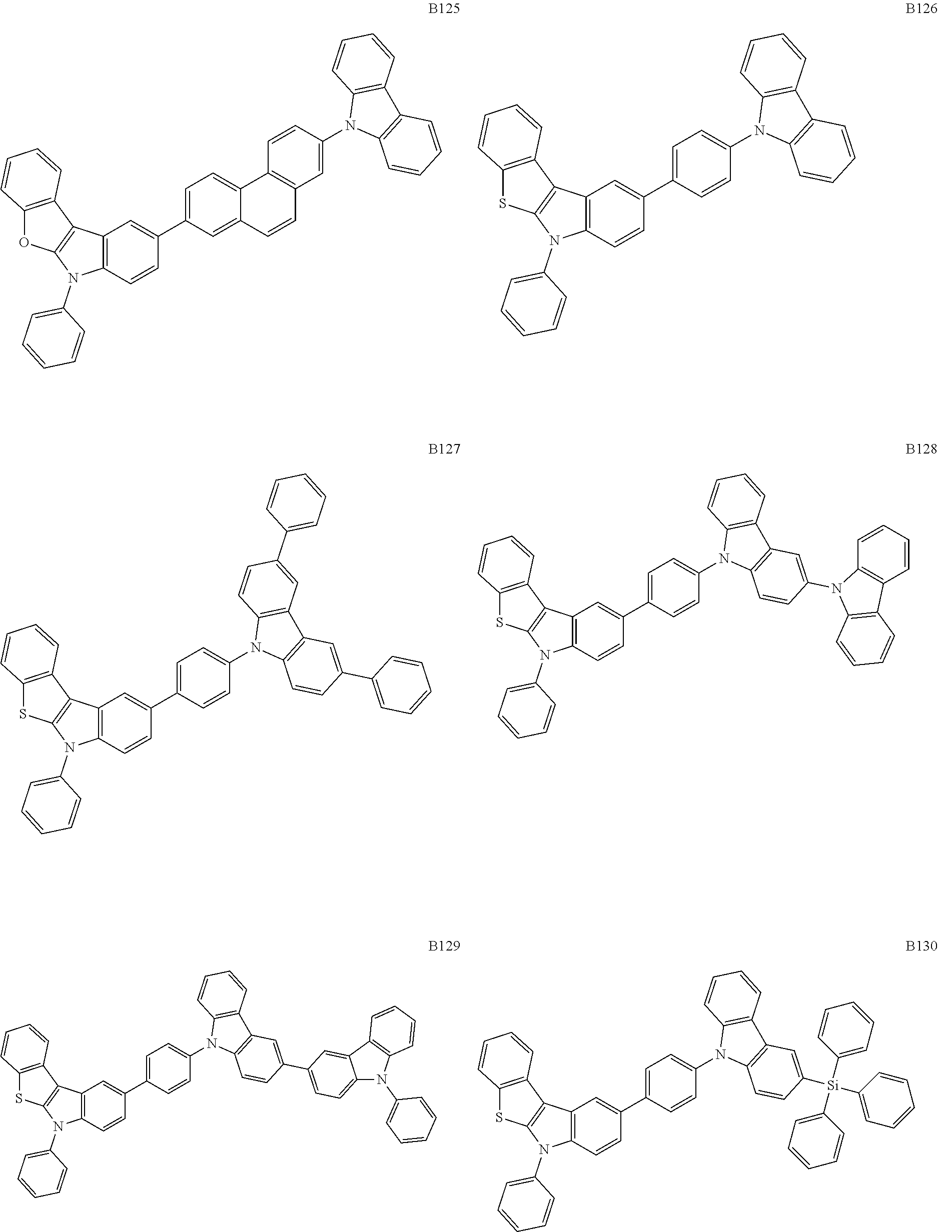

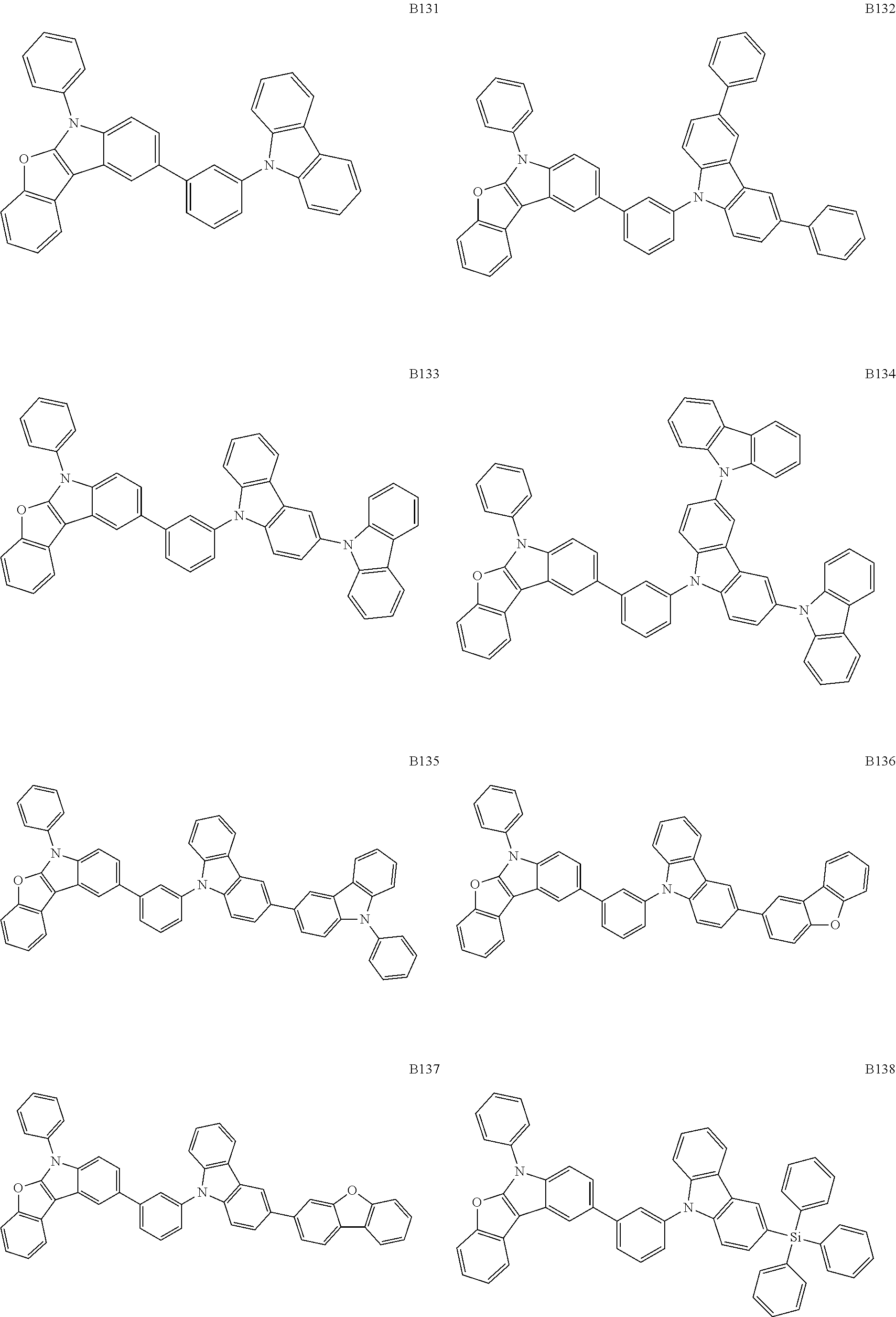

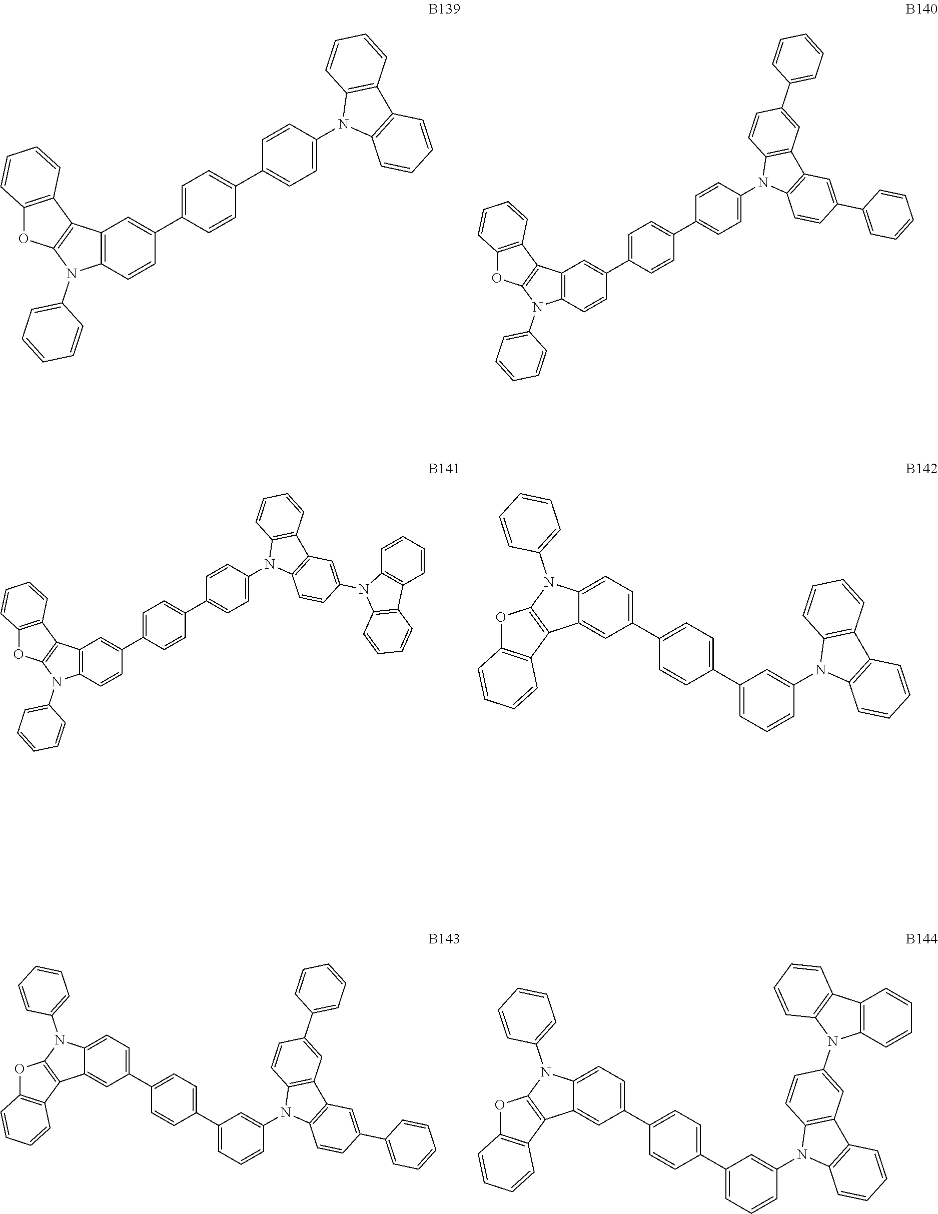

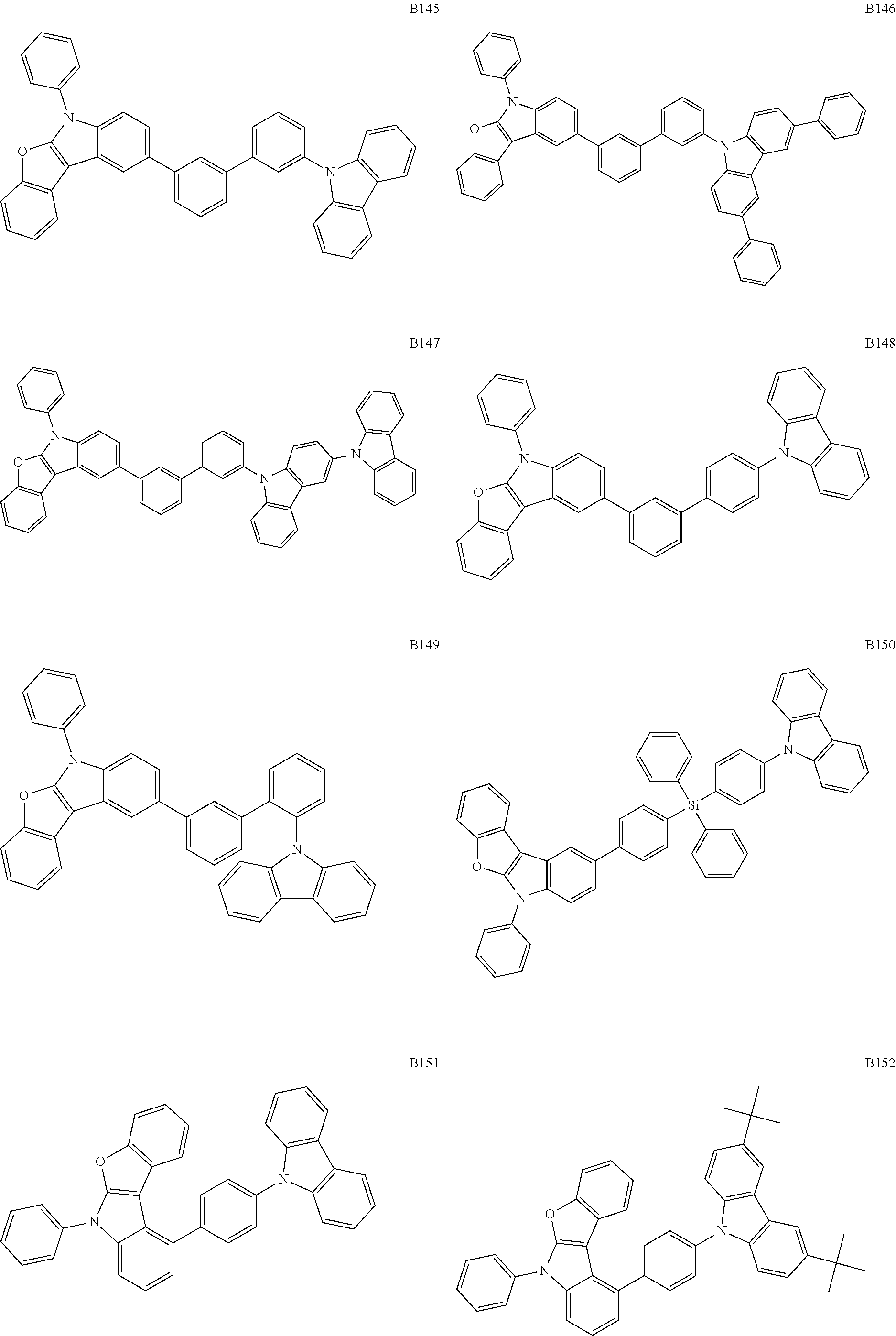

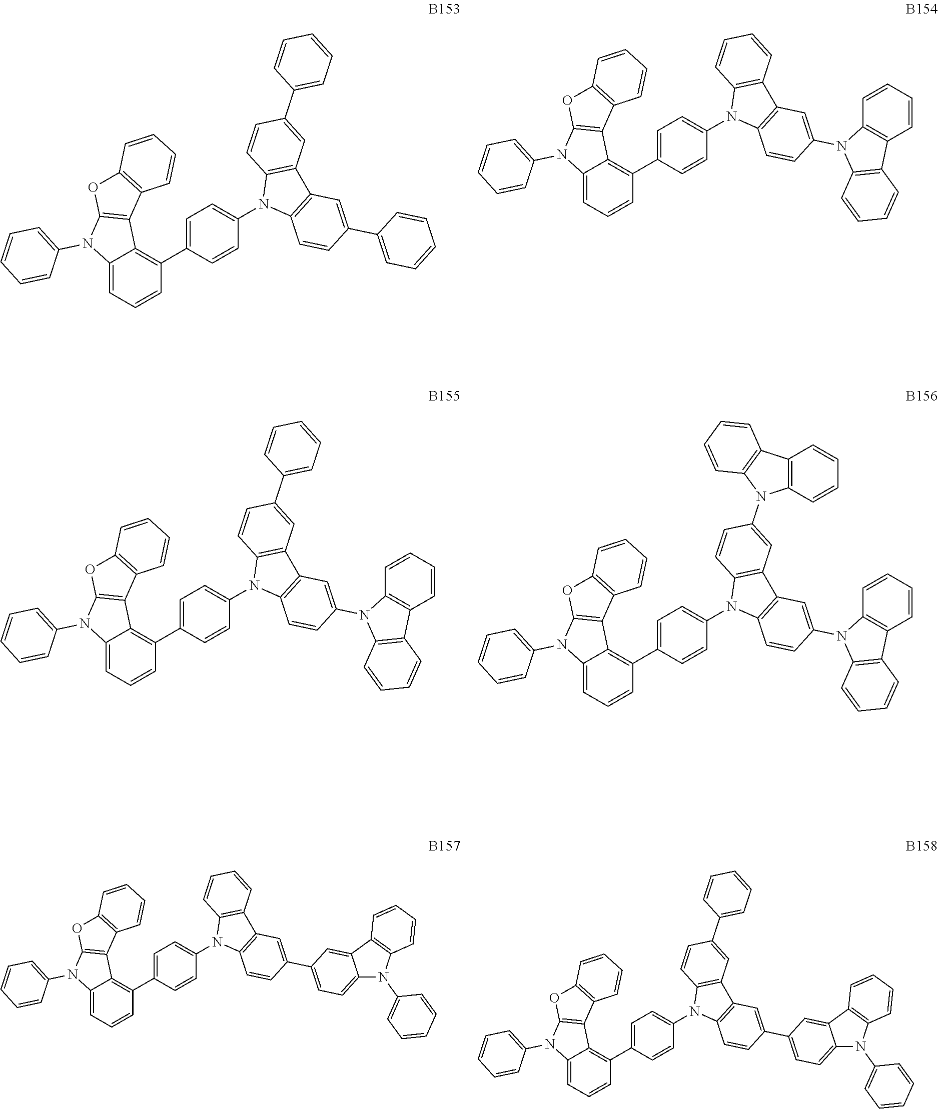

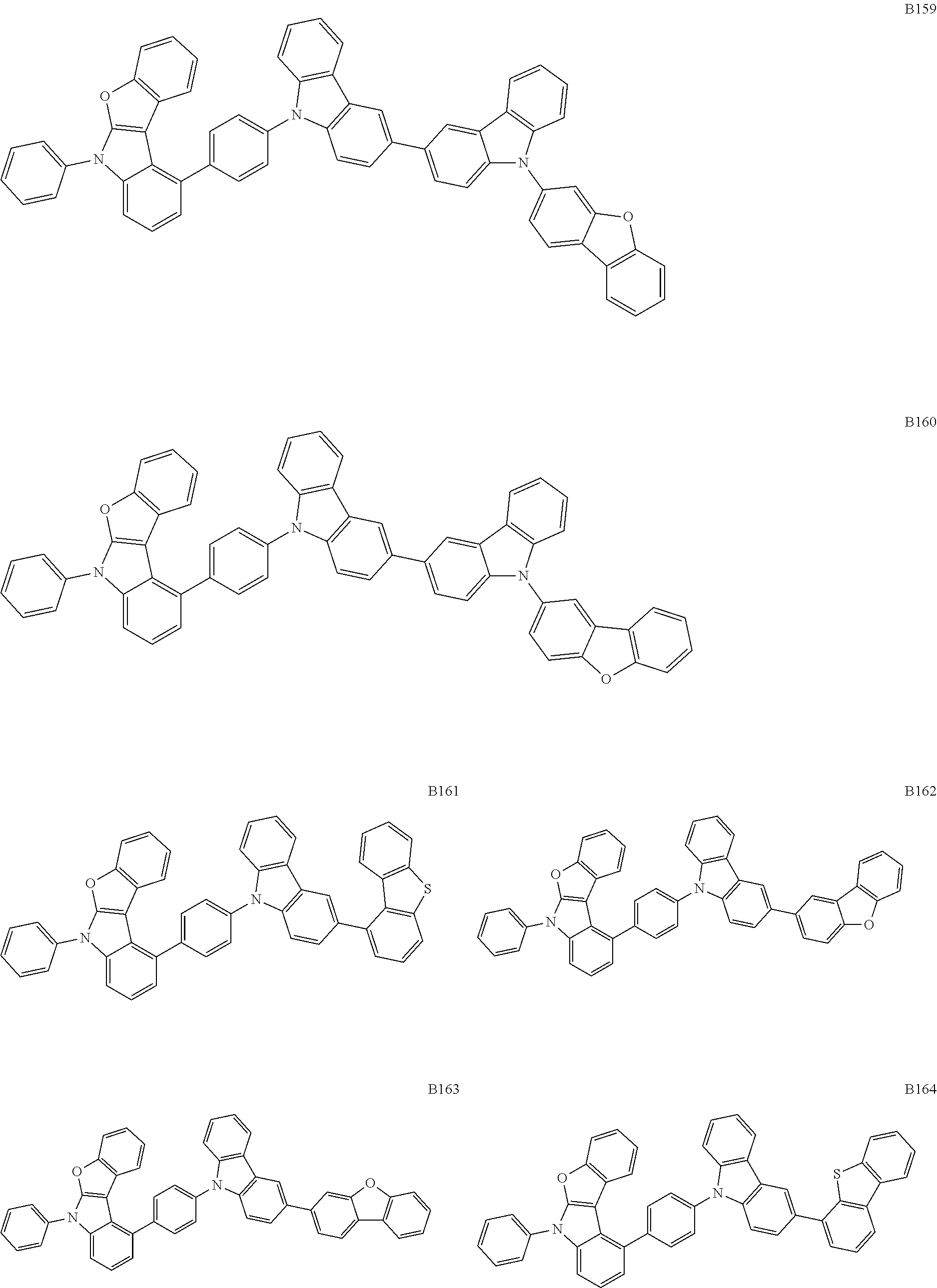

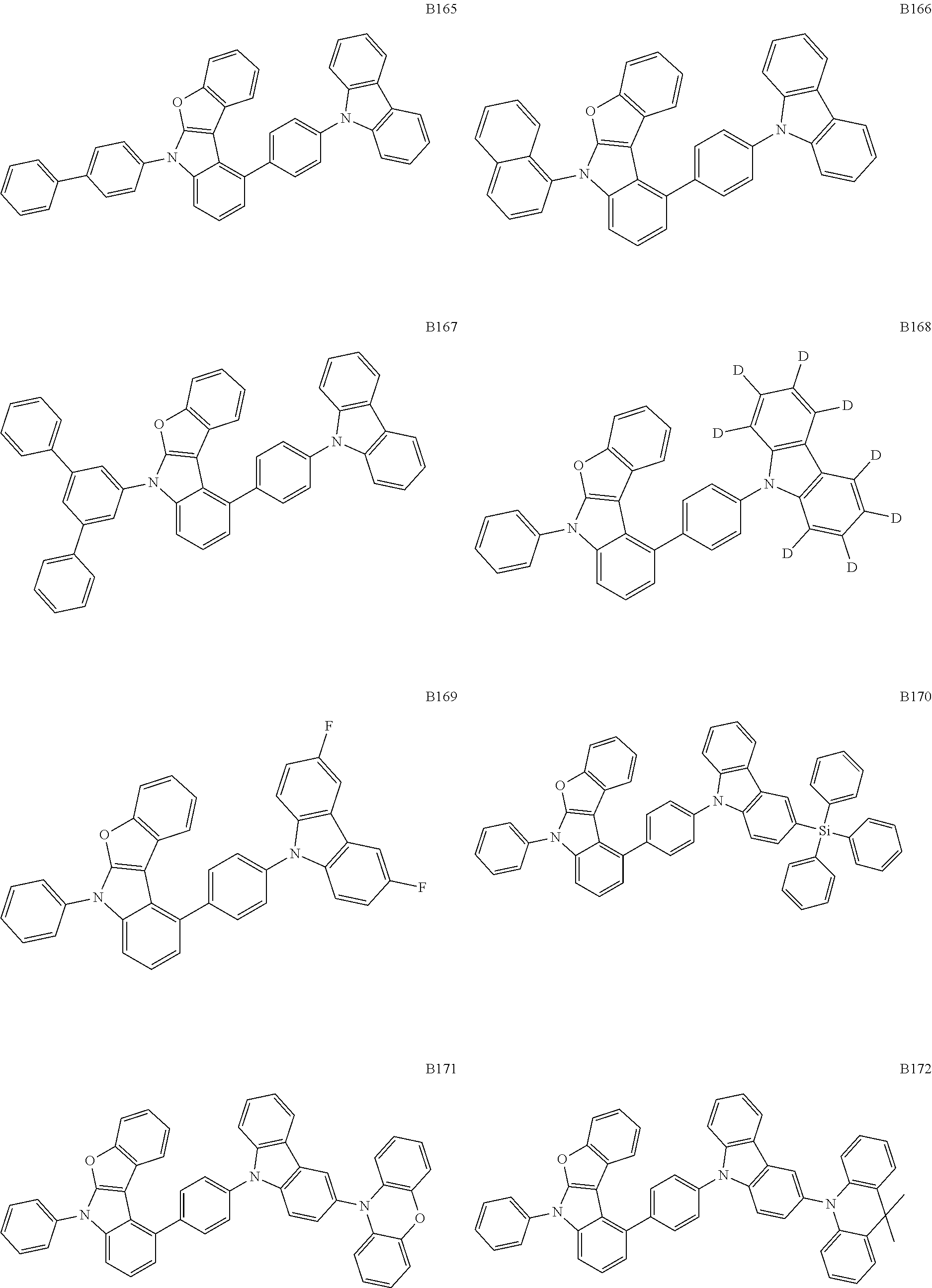

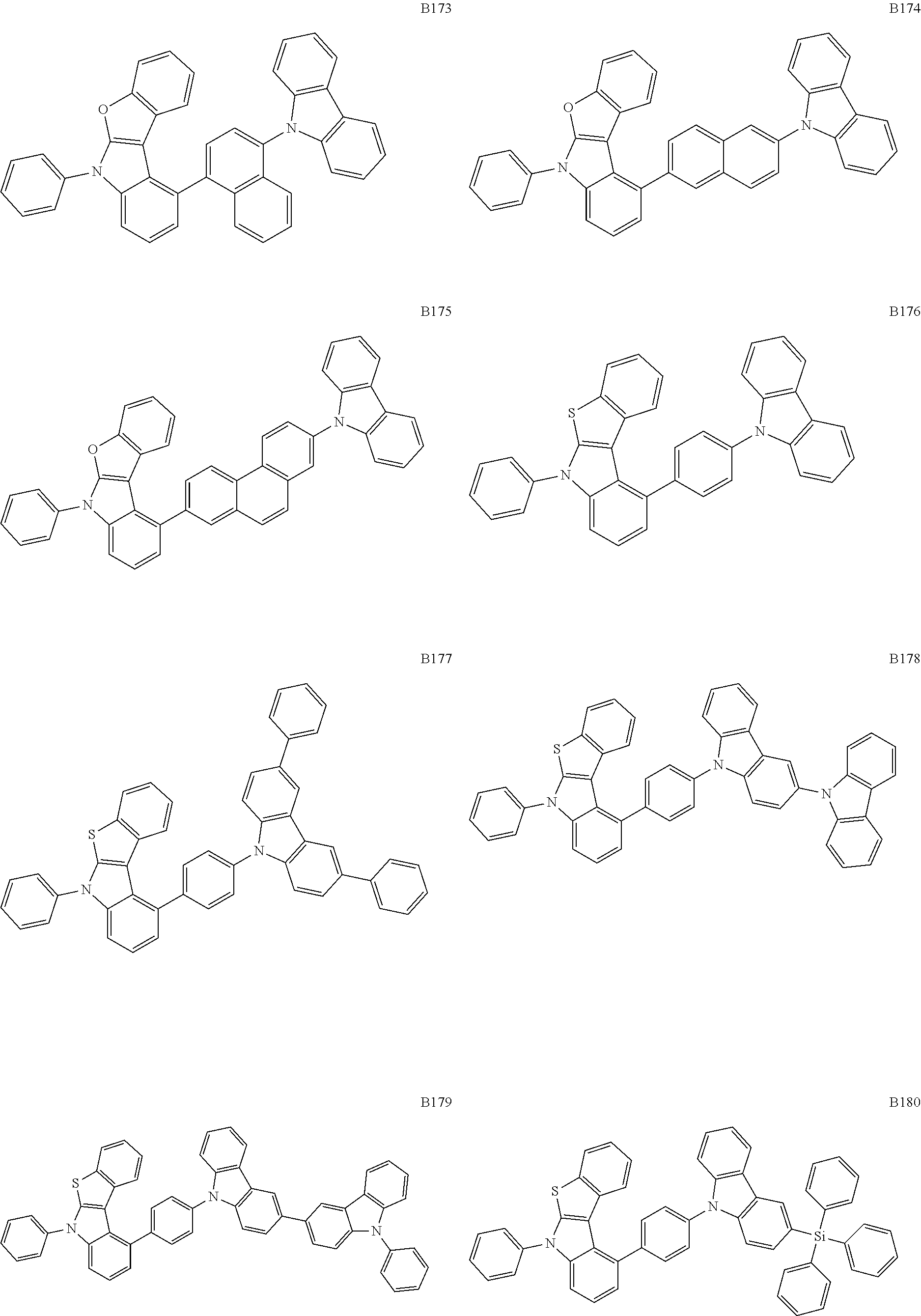

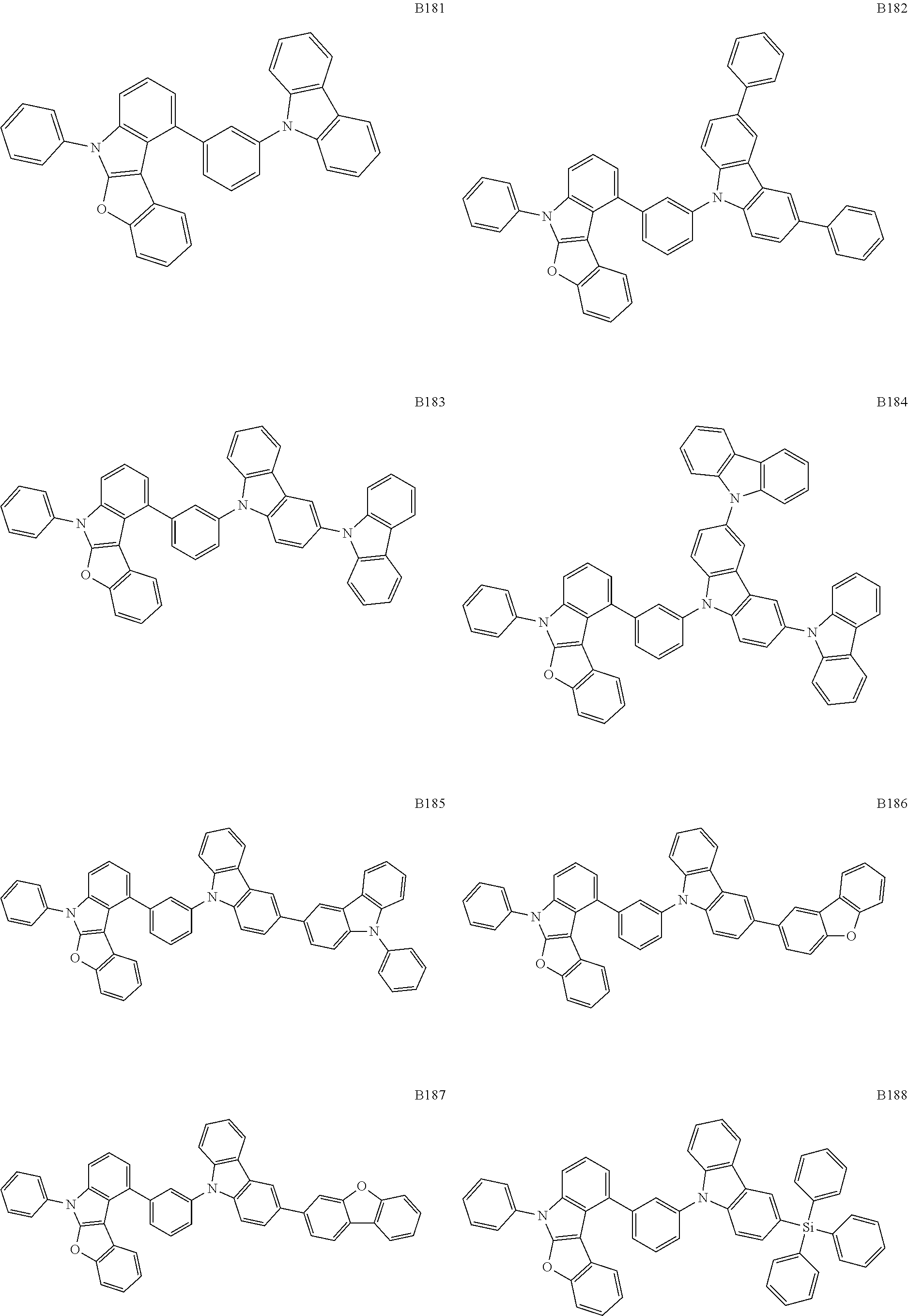

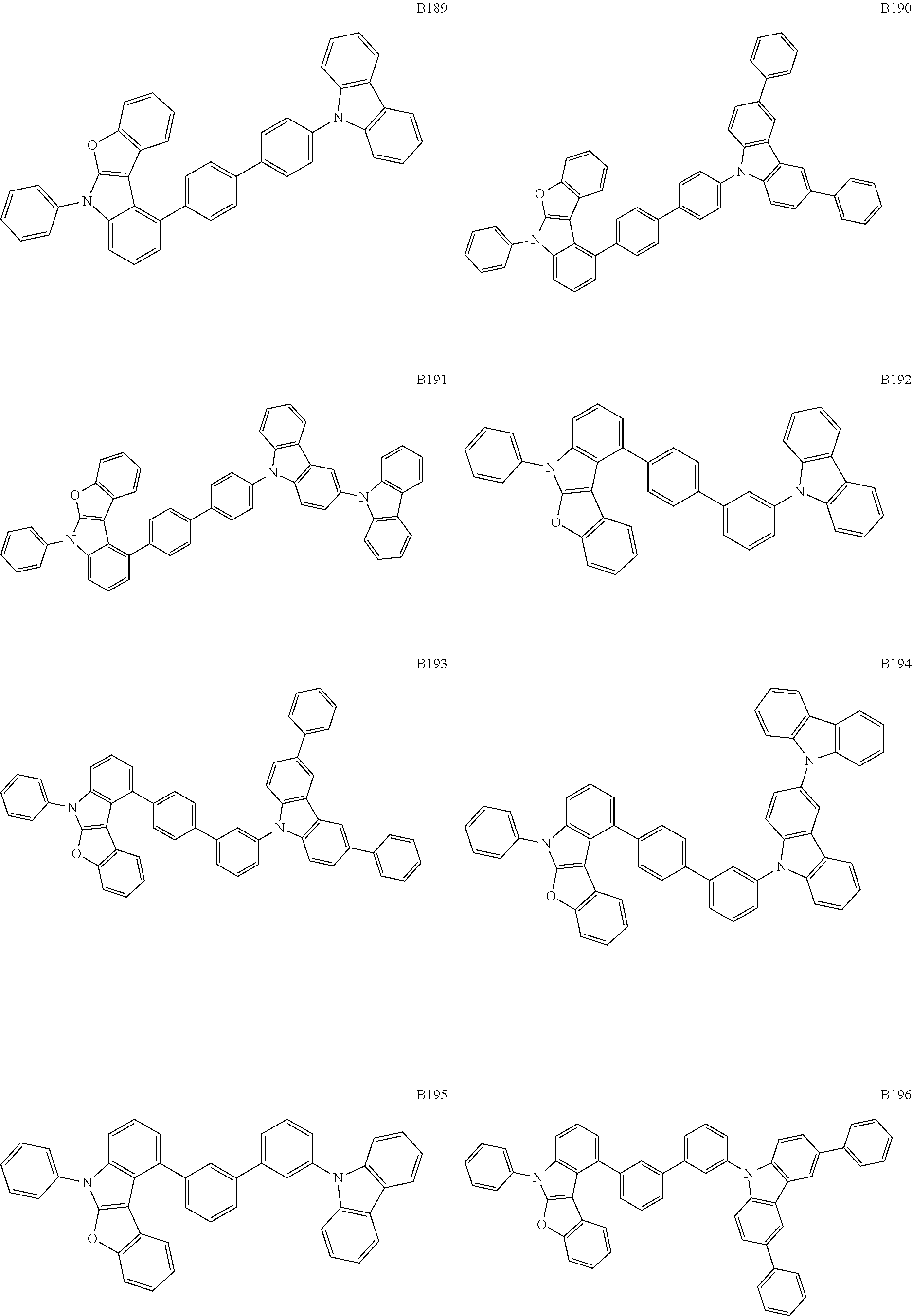

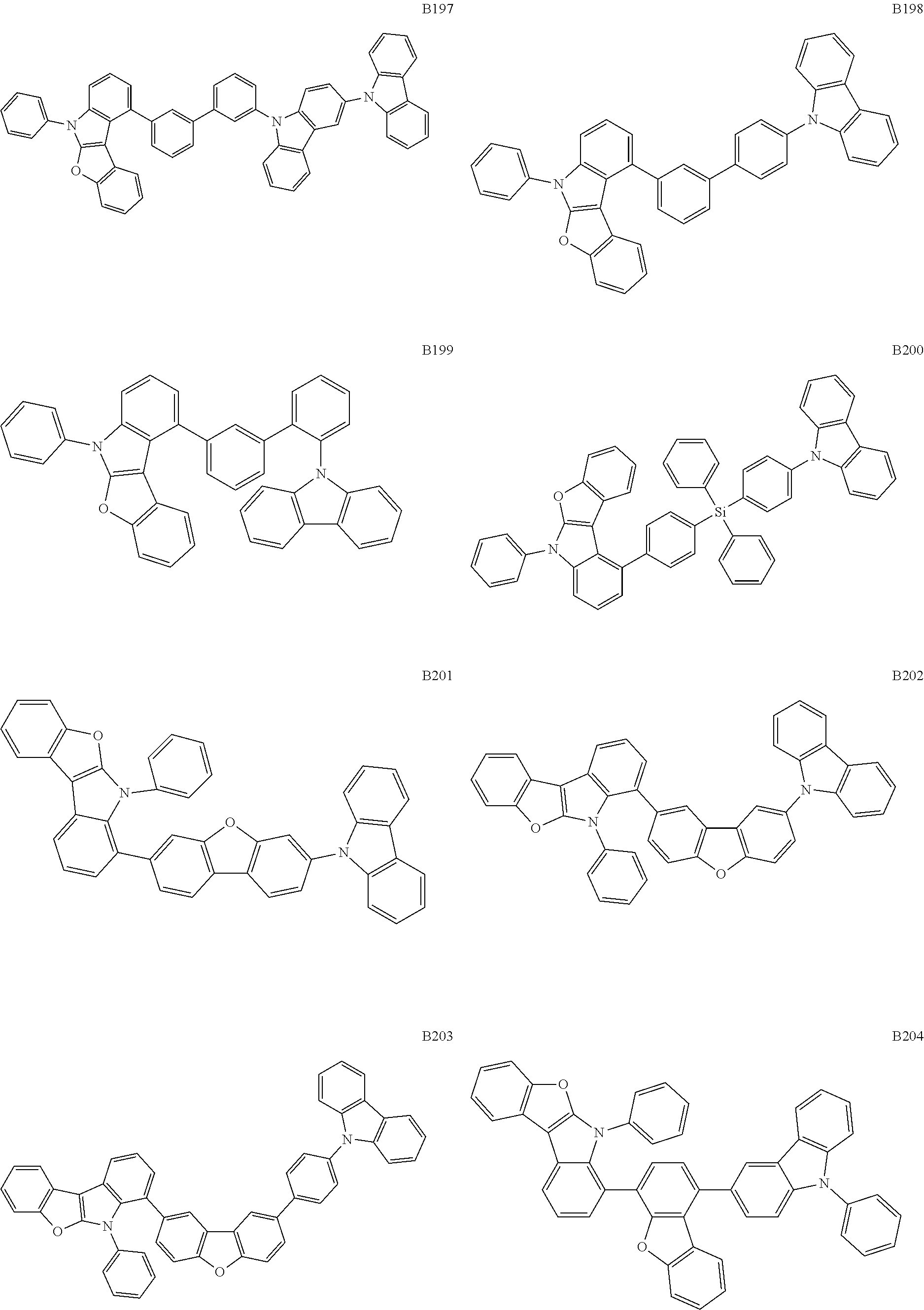

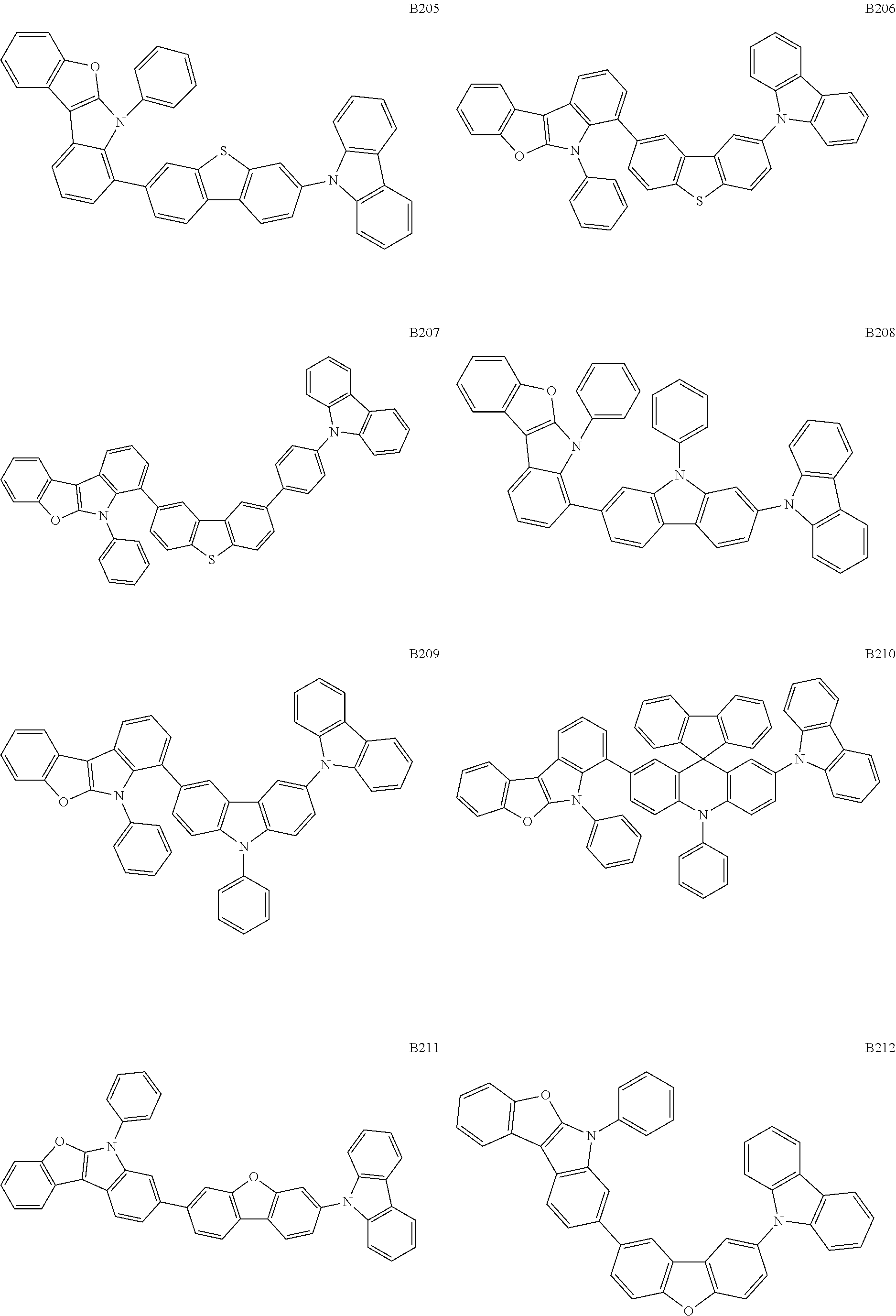

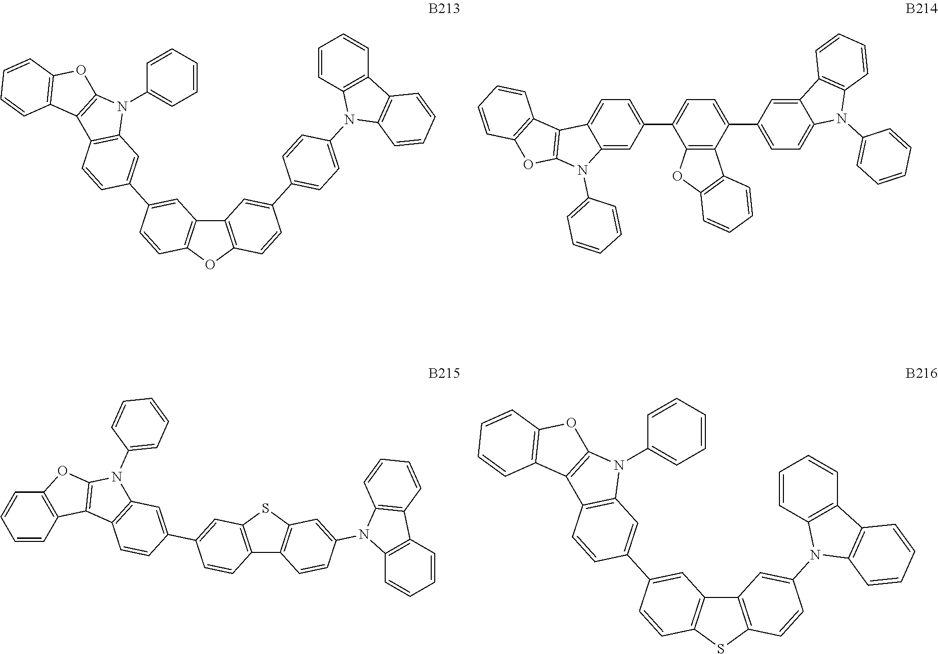 [SMILES: c1ccc(-n2c3cc(-c4ccc5oc6ccc(-c7ccc(-n8c9ccccc9c9ccccc98)cc7)cc6c5c4)ccc3c3c4ccccc4oc32)cc1 ; c1ccc(-n2c3ccccc3c3cc(-c4ccc(-c5ccc6c7c8ccccc8oc7n(-c7ccccc7)c6c5)c5oc6ccccc6c45)ccc32)cc1 ; c1ccc(-n2c3cc(-c4ccc5c(c4)sc4cc(-n6c7ccccc7c7ccccc76)ccc45)ccc3c3c4ccccc4oc32)cc1 ; c1ccc(-n2c3cc(-c4ccc5sc6ccc(-n7c8ccccc8c8ccccc87)cc6c5c4)ccc3c3c4ccccc4oc32)cc1]

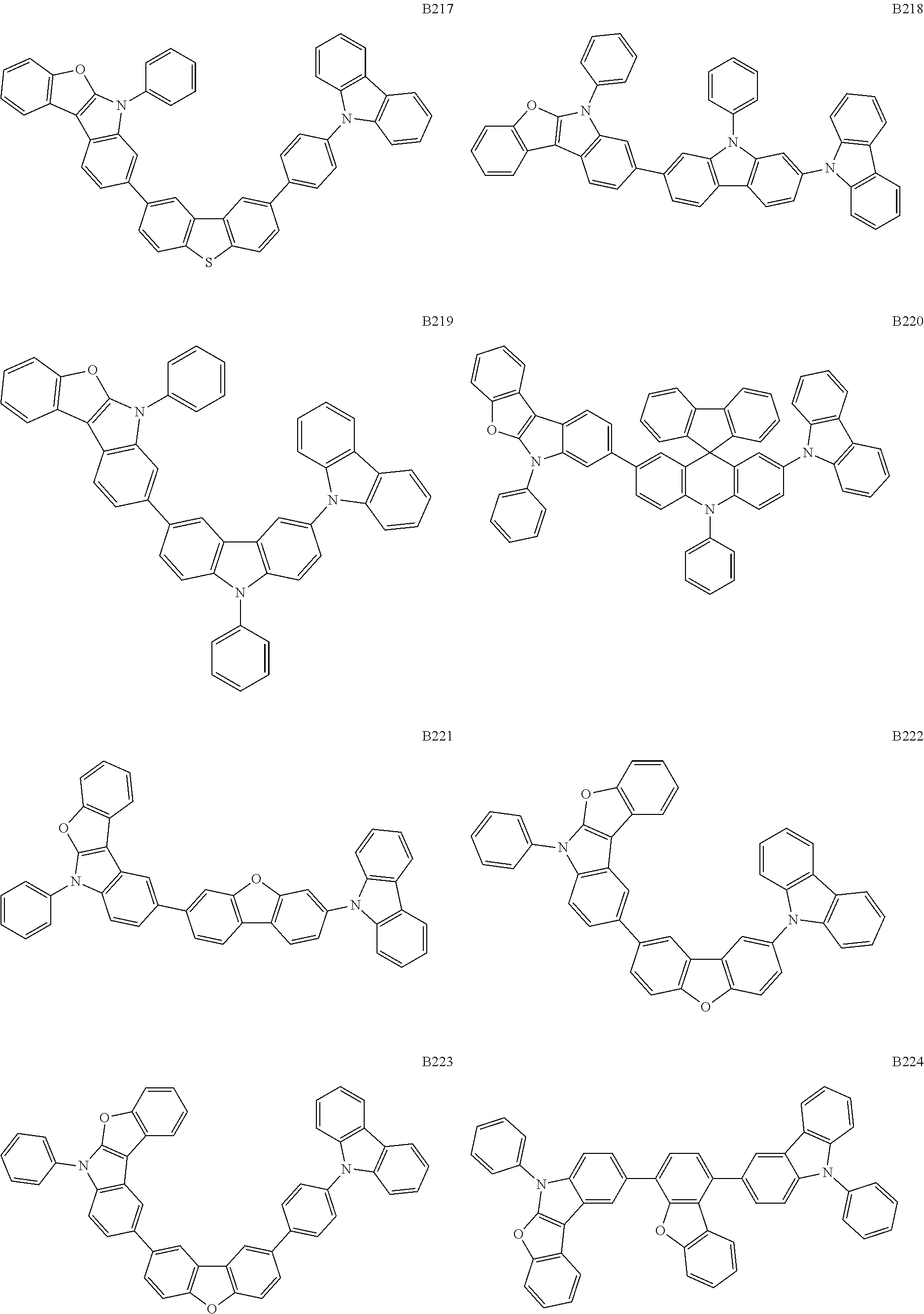

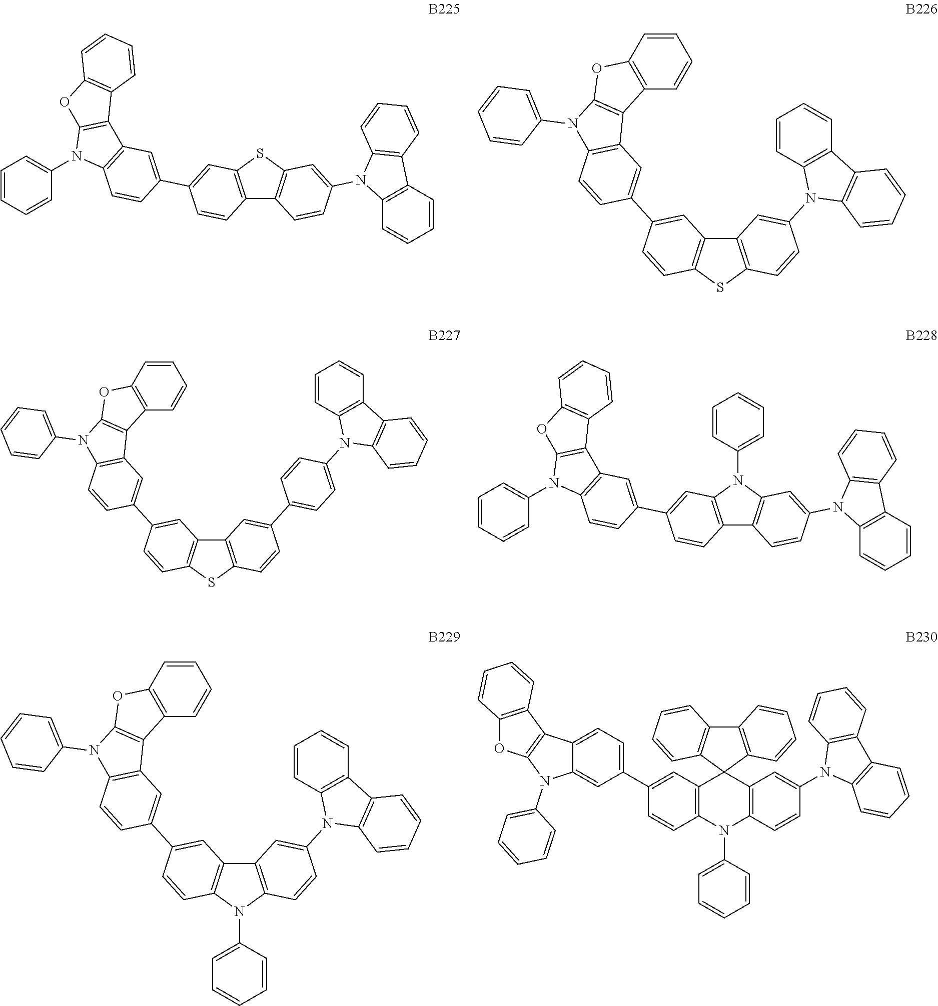

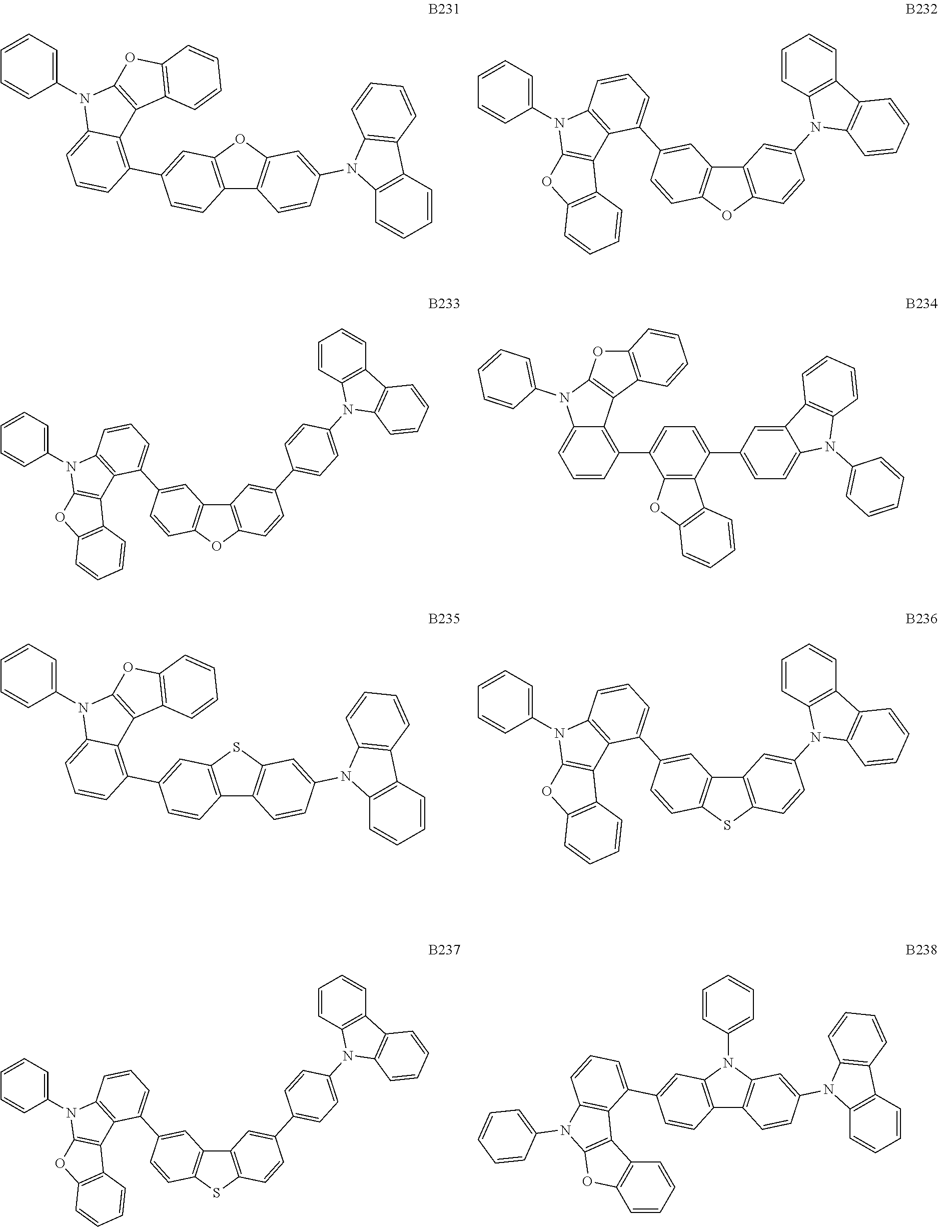

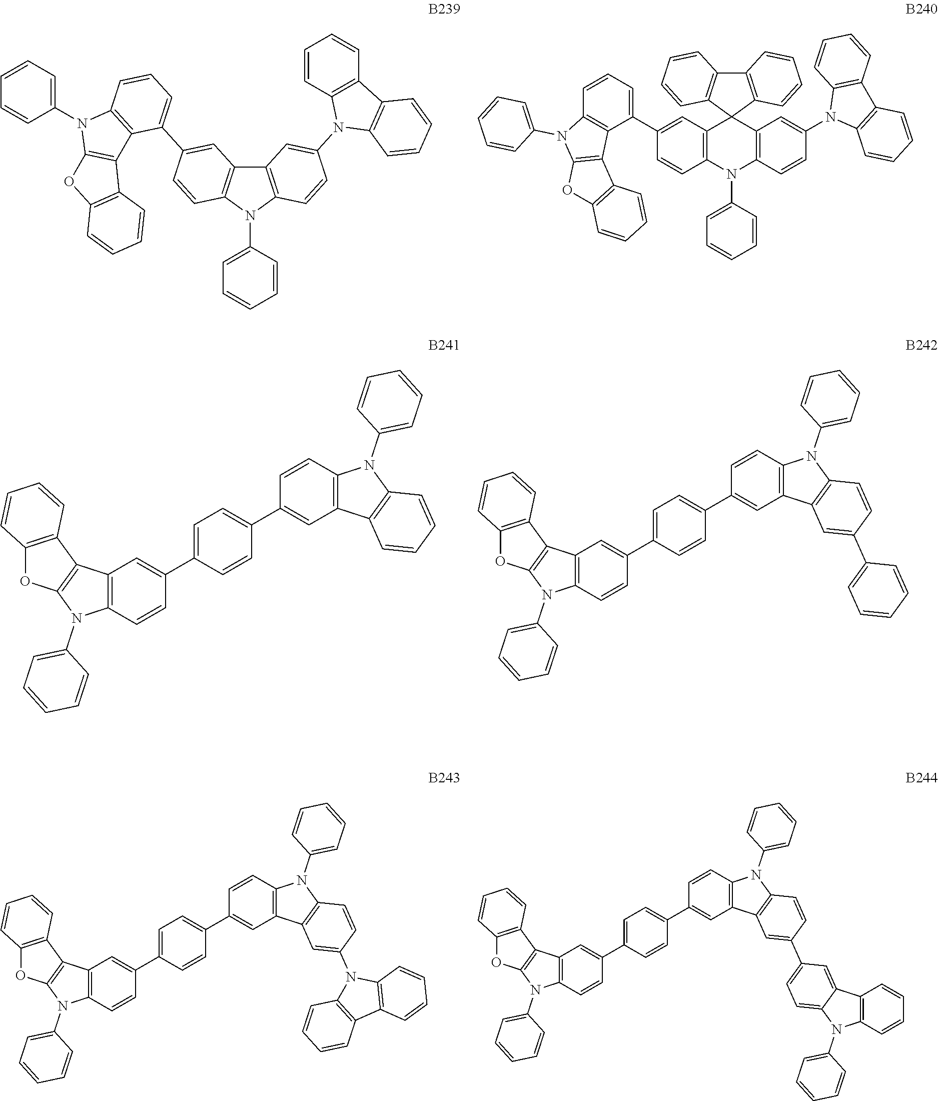

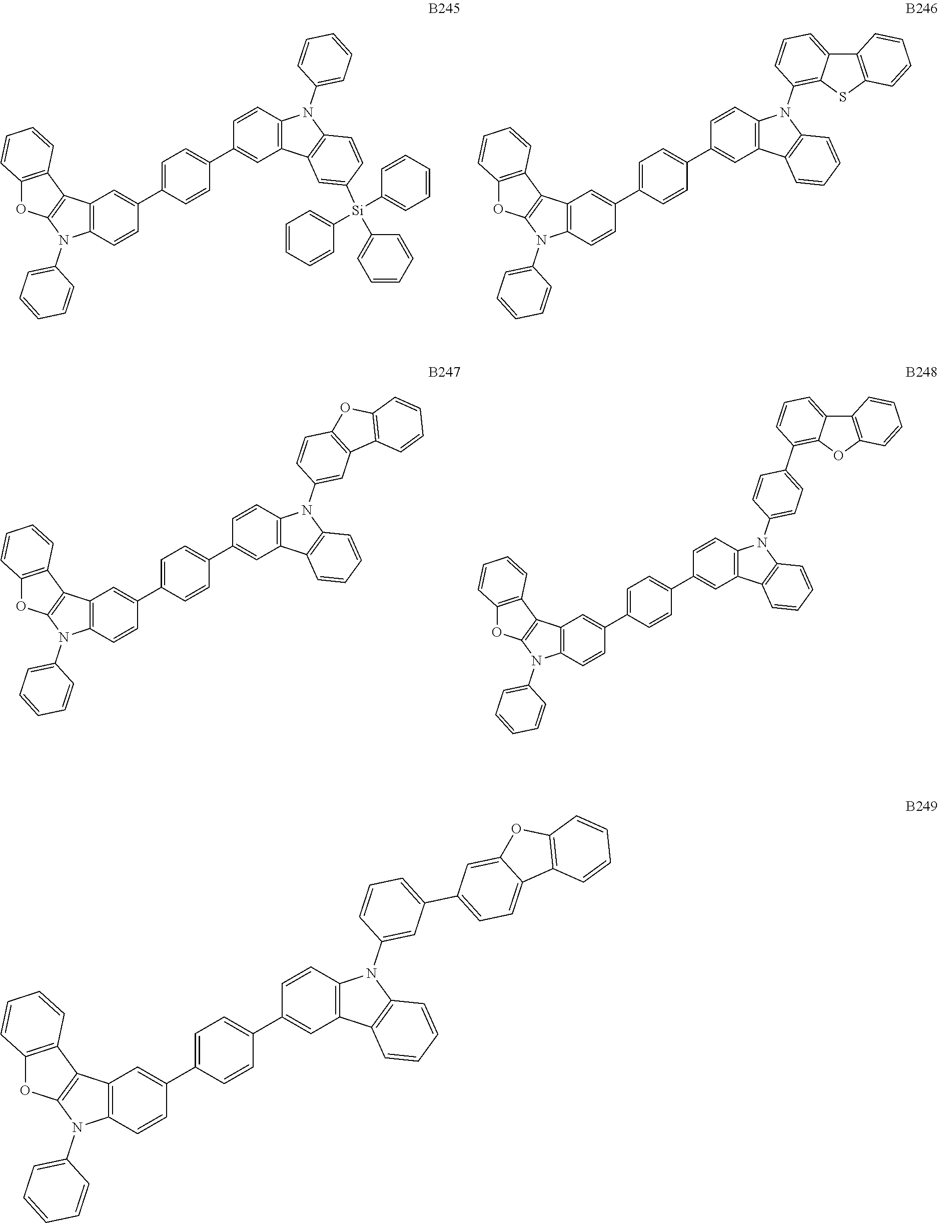

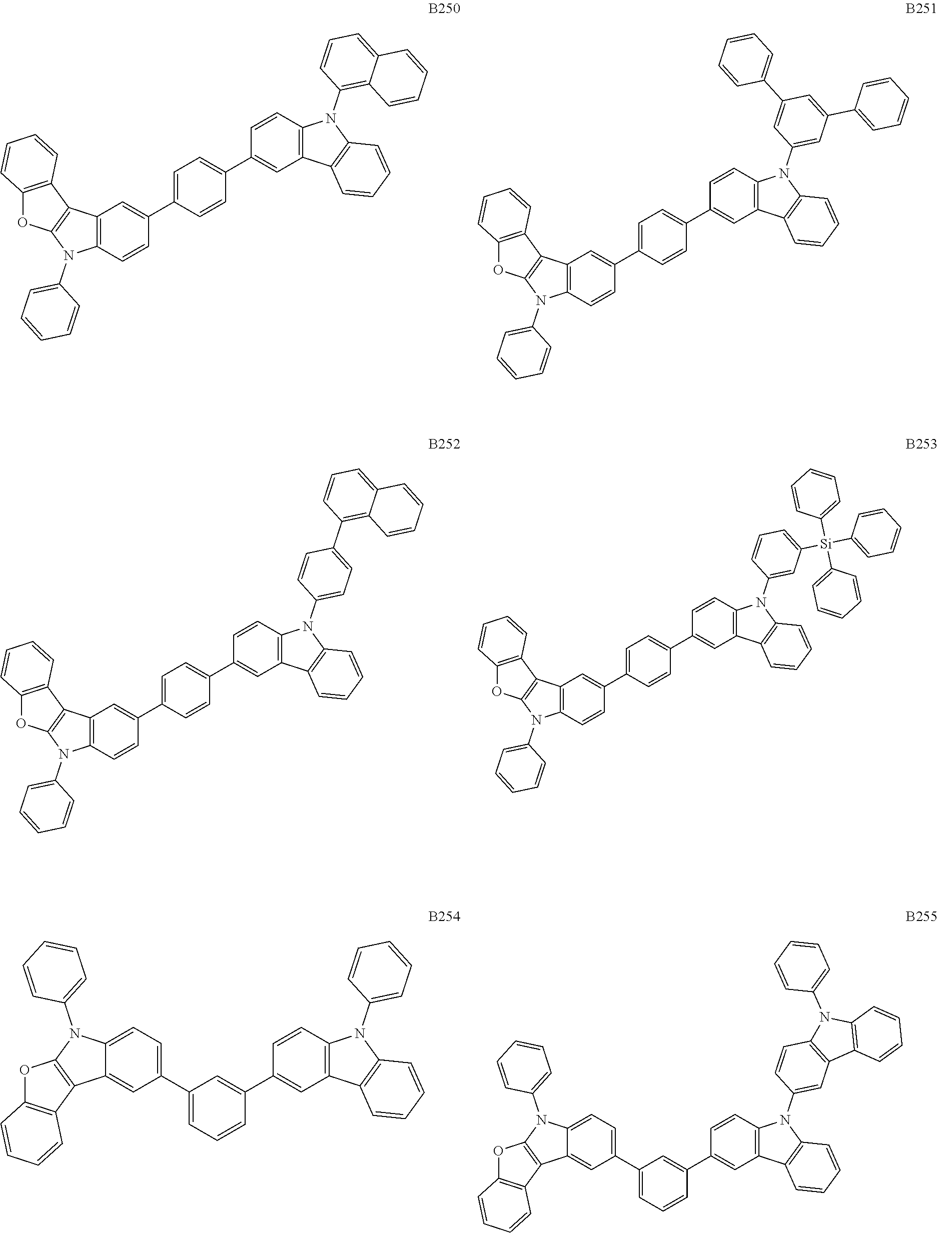

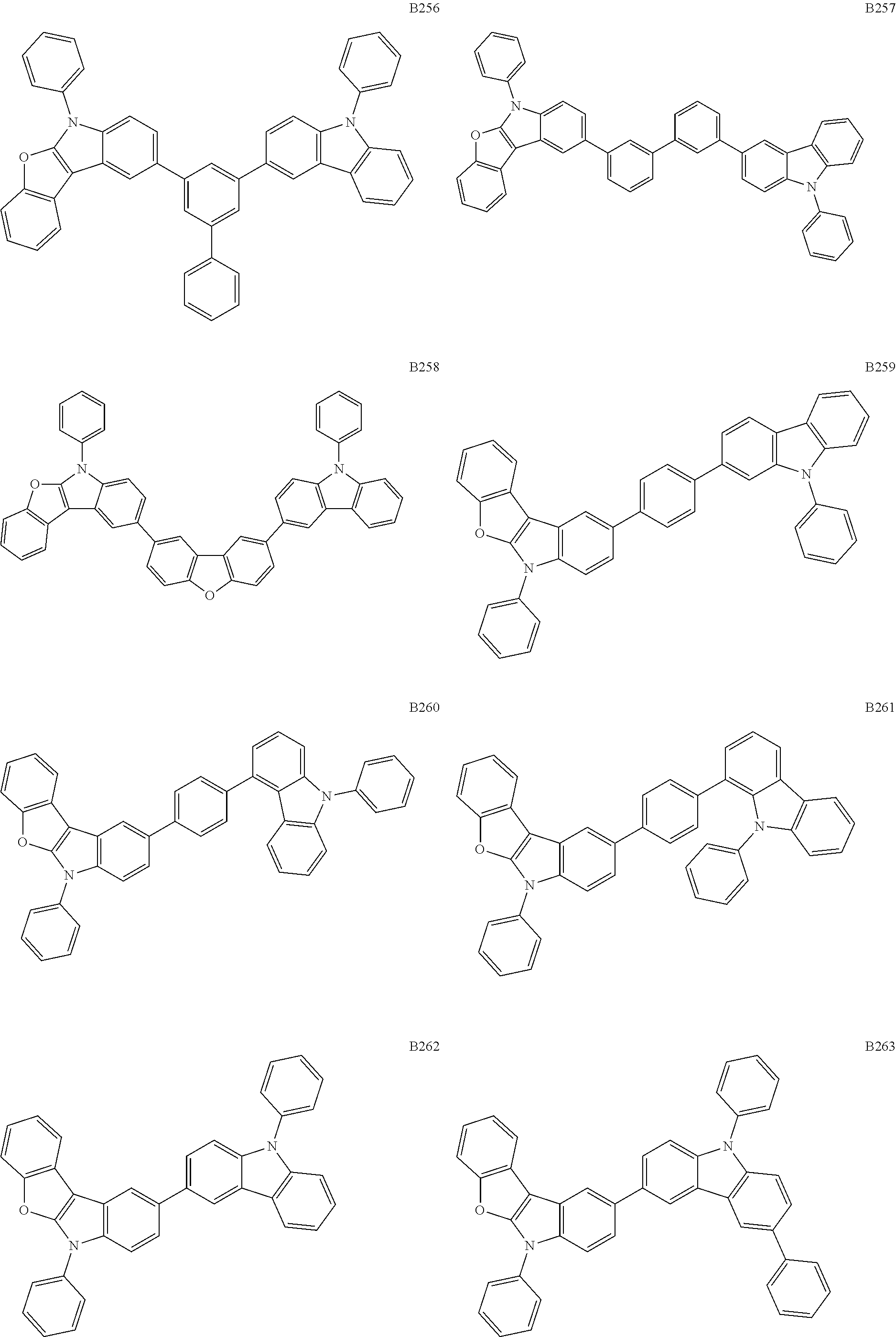

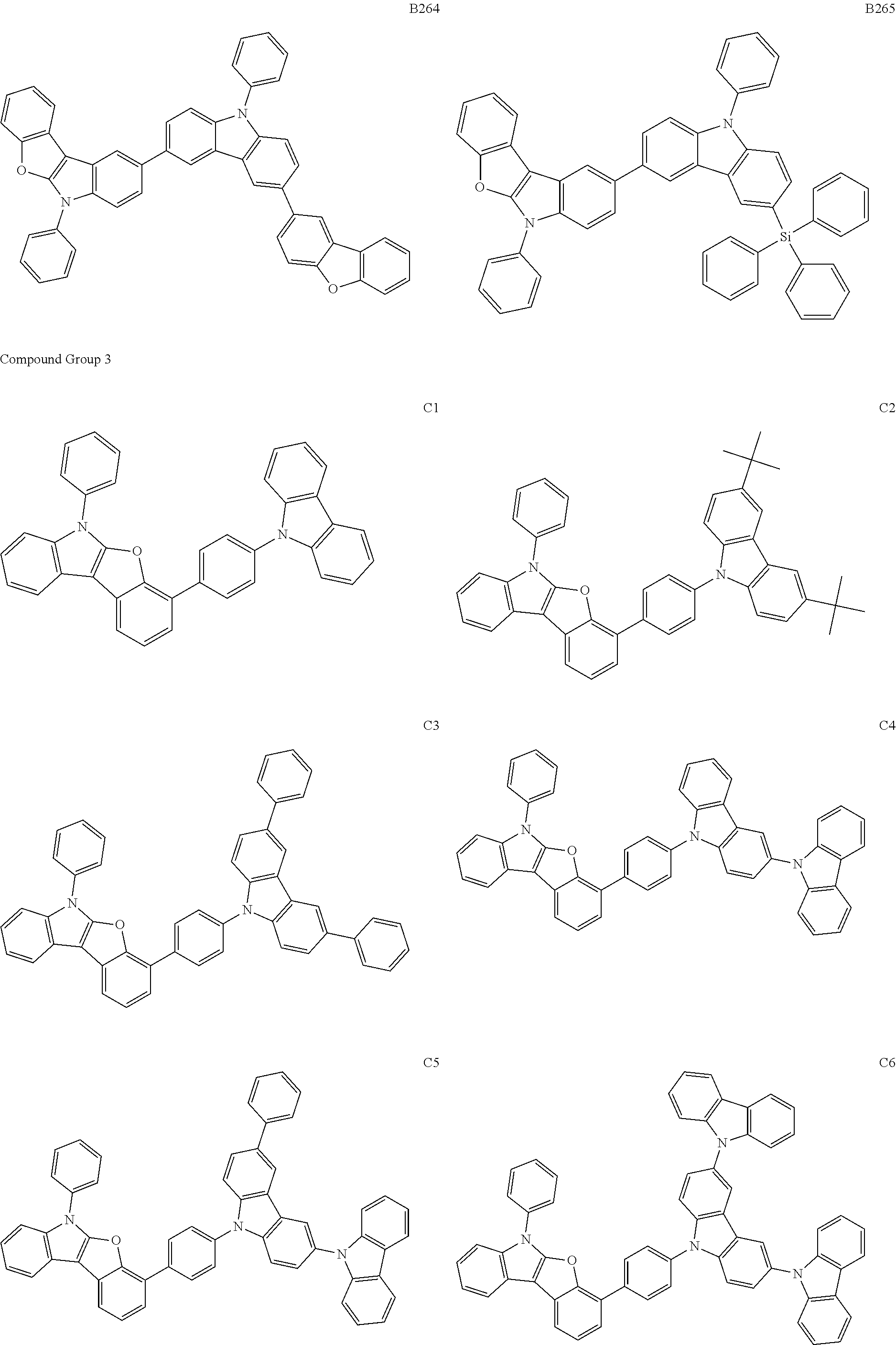

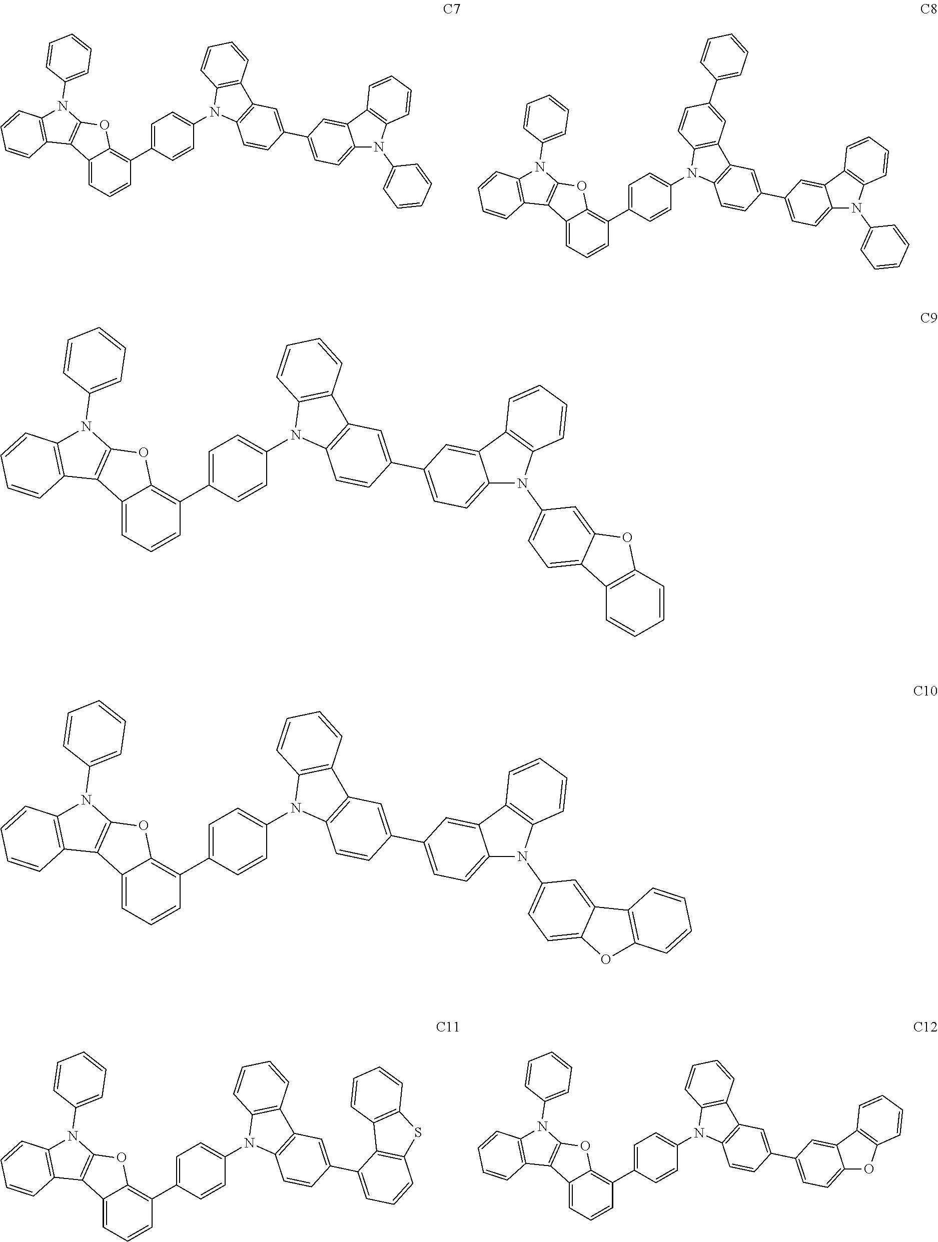

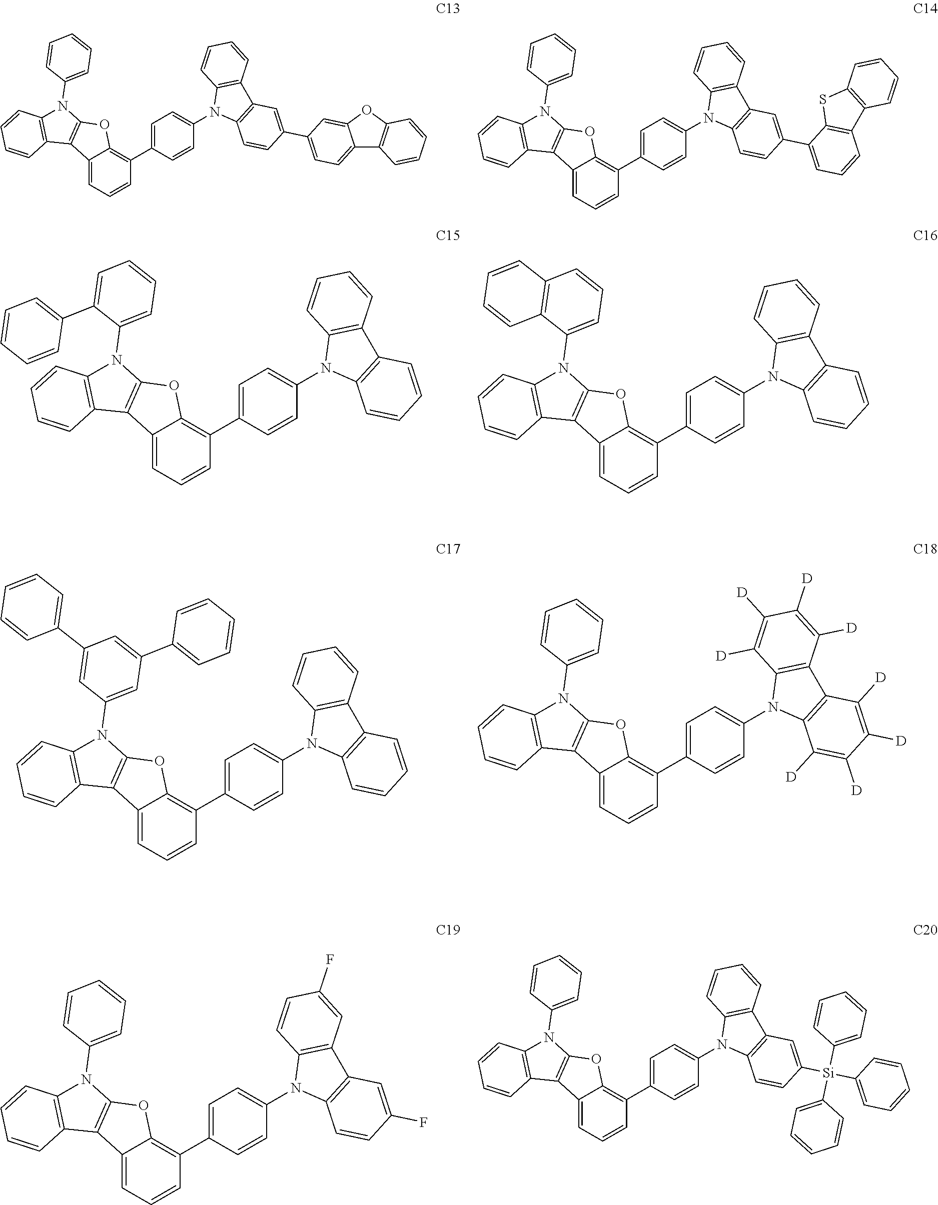

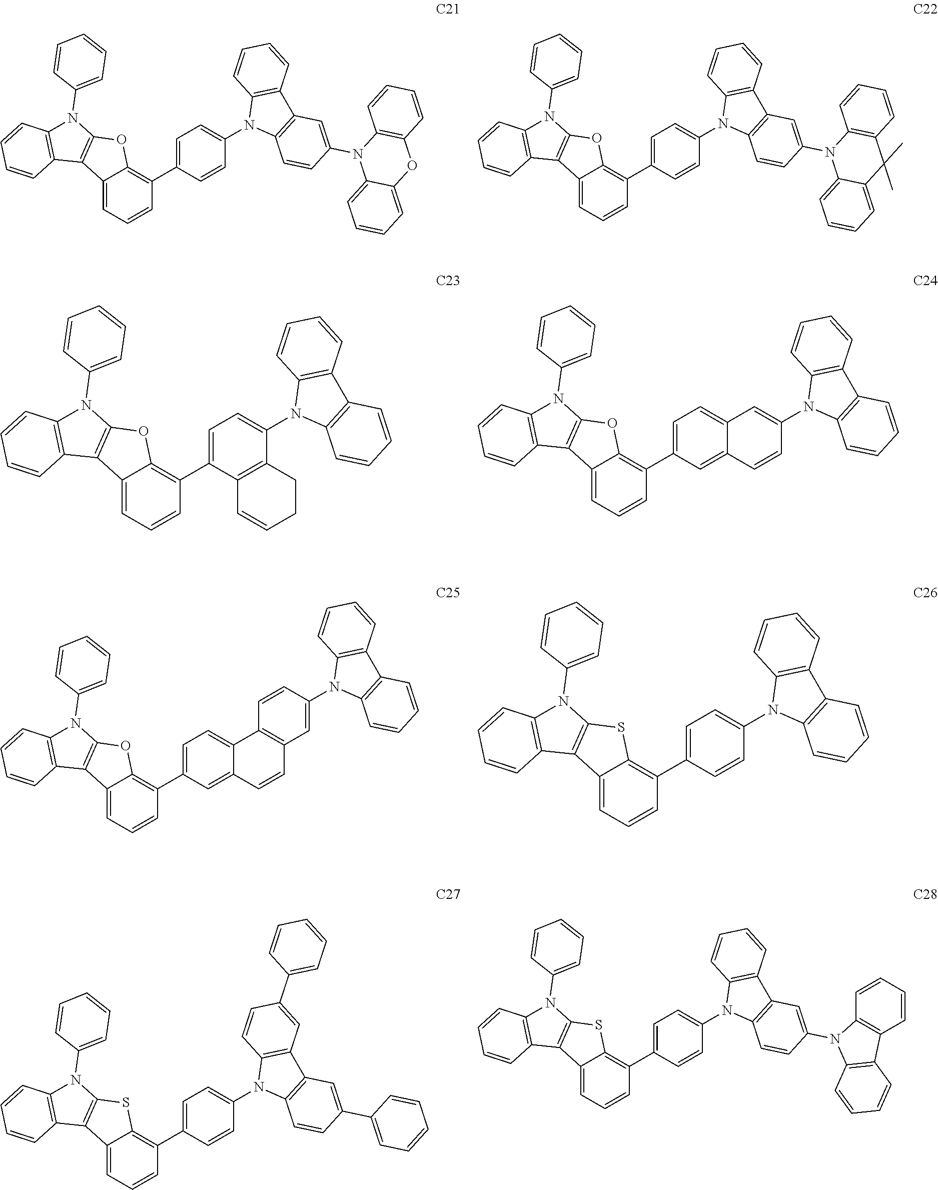

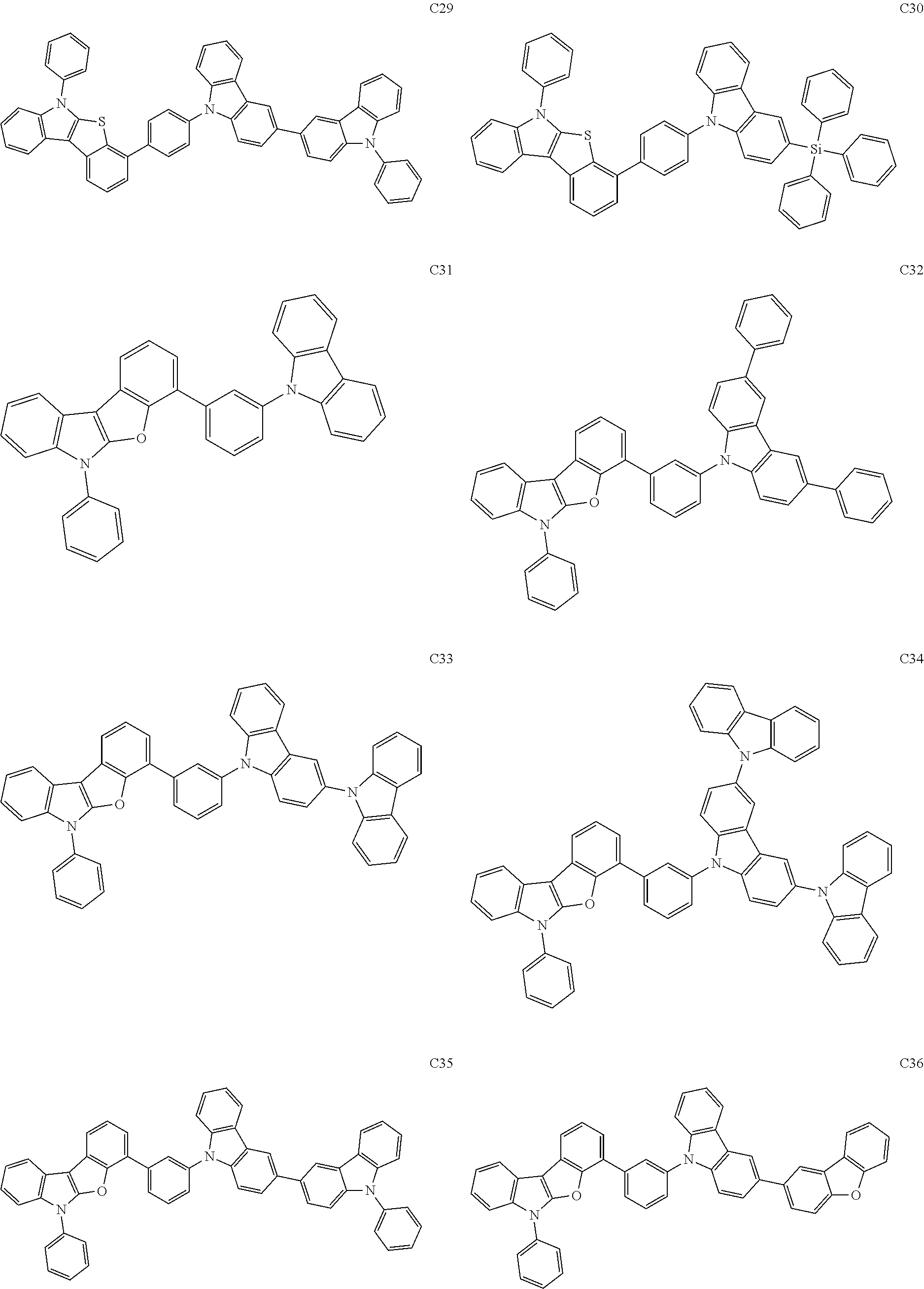

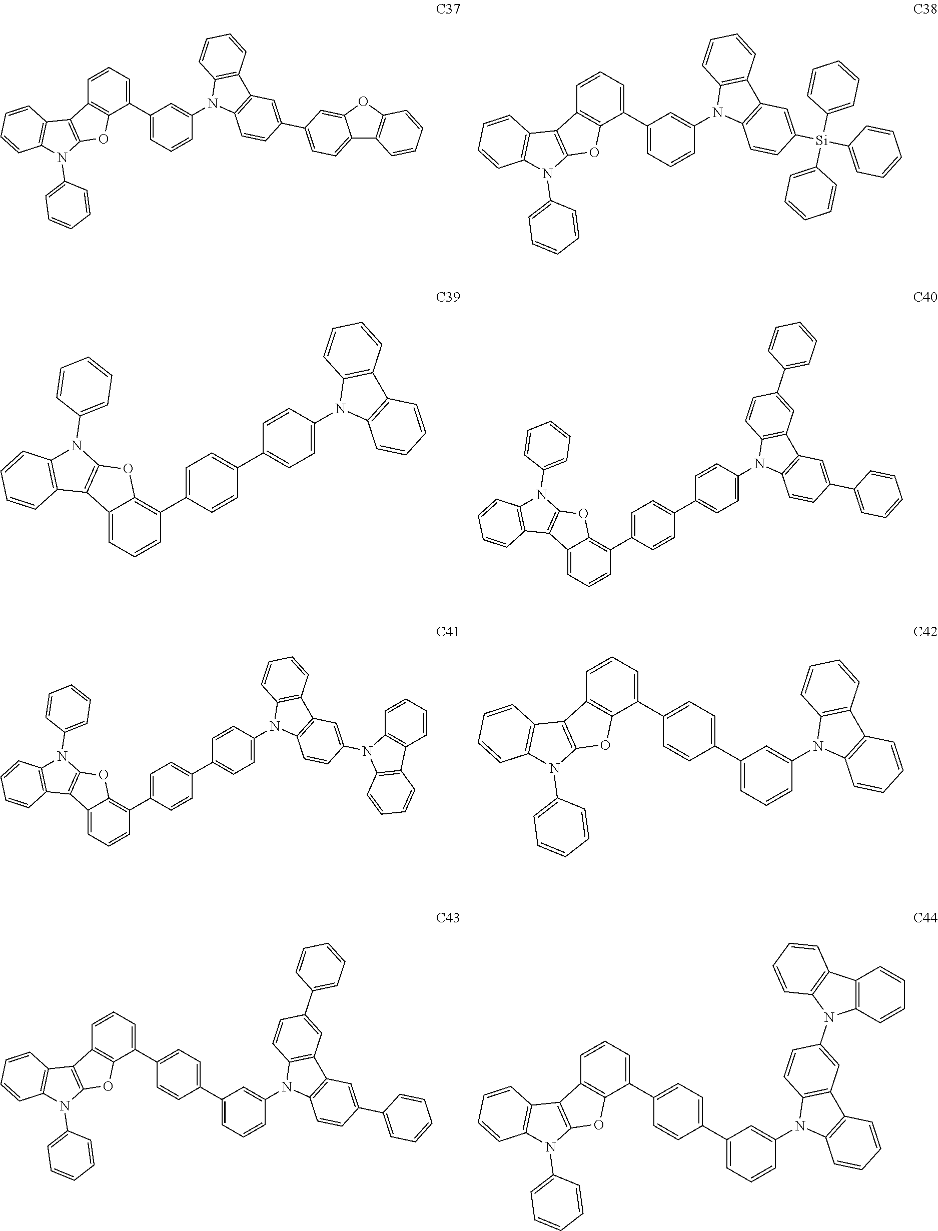

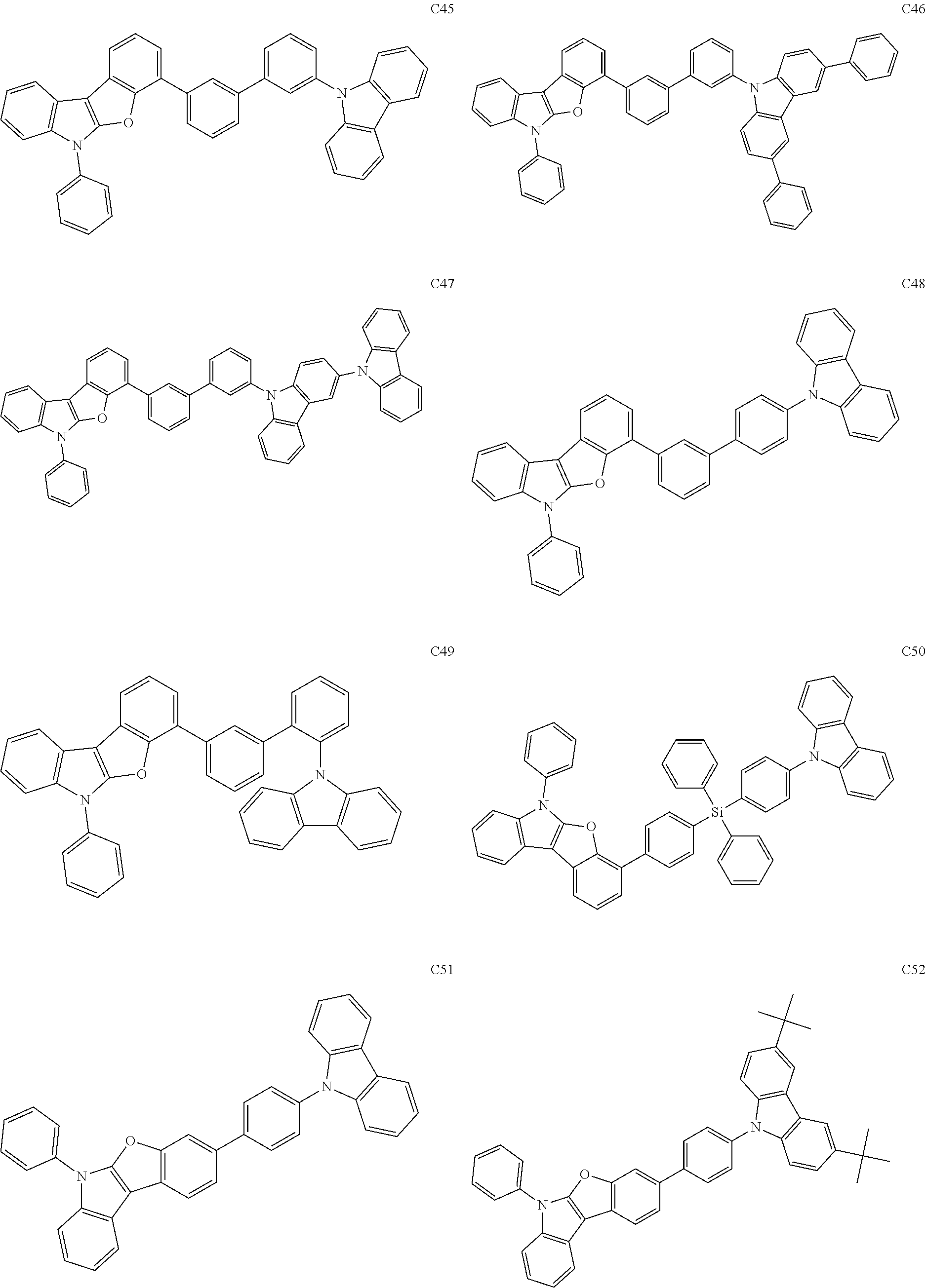

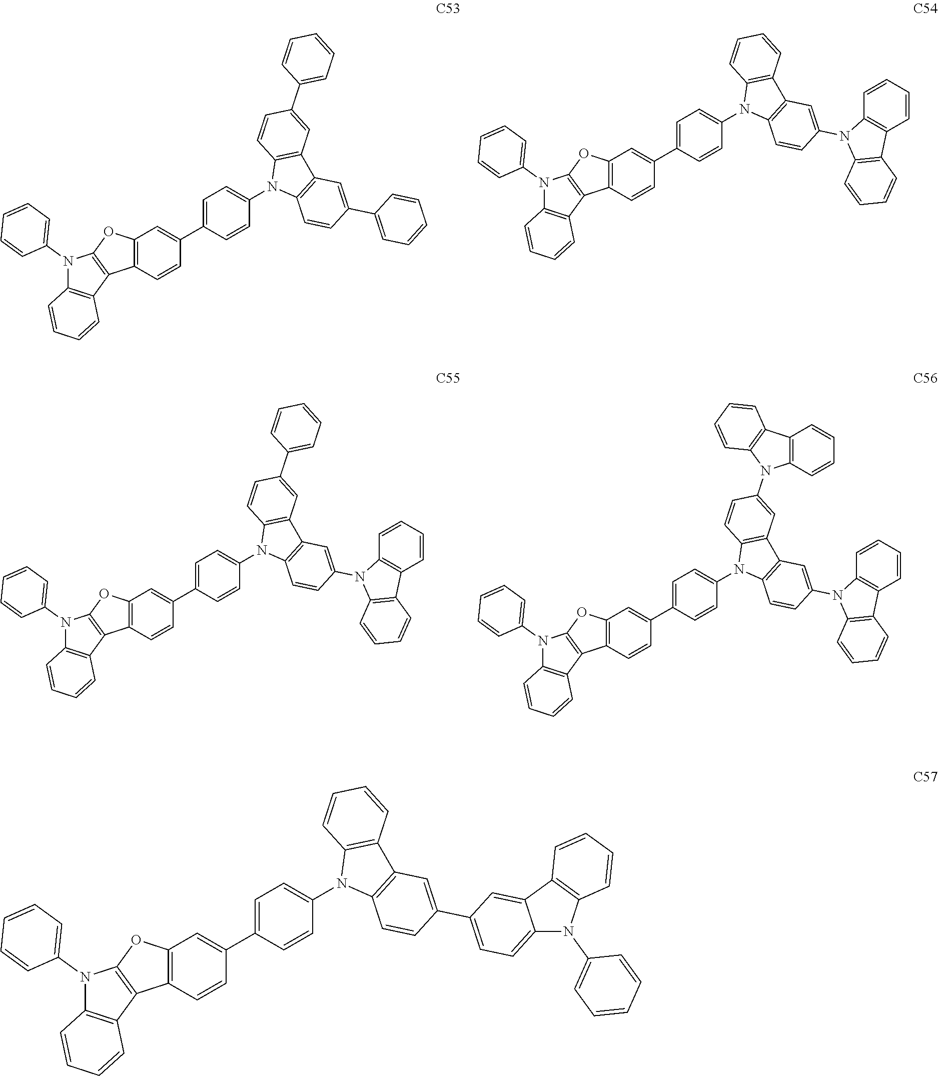

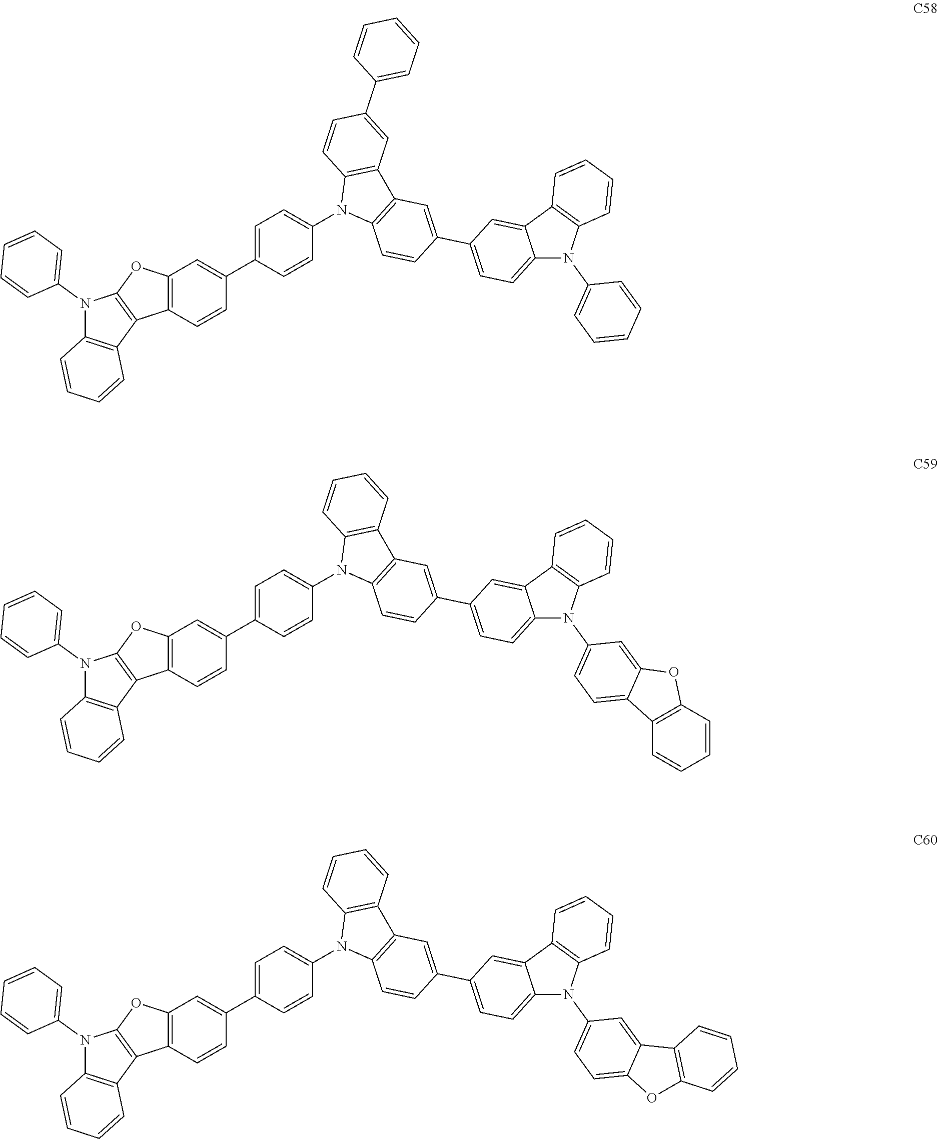 [SMILES: c1ccc(-c2ccc3c(c2)c2cc(-c4ccc5c(c4)c4ccccc4n5-c4ccccc4)ccc2n3-c2ccc(-c3ccc4c(c3)oc3c4c4ccccc4n3-c3ccccc3)cc2)cc1 ; c1ccc(-n2c3ccccc3c3c4ccc(-c5ccc(-n6c7ccccc7c7cc(-c8ccc9c(c8)c8ccccc8n9-c8ccc9c(c8)oc8ccccc89)ccc76)cc5)cc4oc32)cc1 ; c1ccc(-n2c3ccccc3c3c4ccc(-c5ccc(-n6c7ccccc7c7cc(-c8ccc9c(c8)c8ccccc8n9-c8ccc9oc%10ccccc%10c9c8)ccc76)cc5)cc4oc32)cc1]

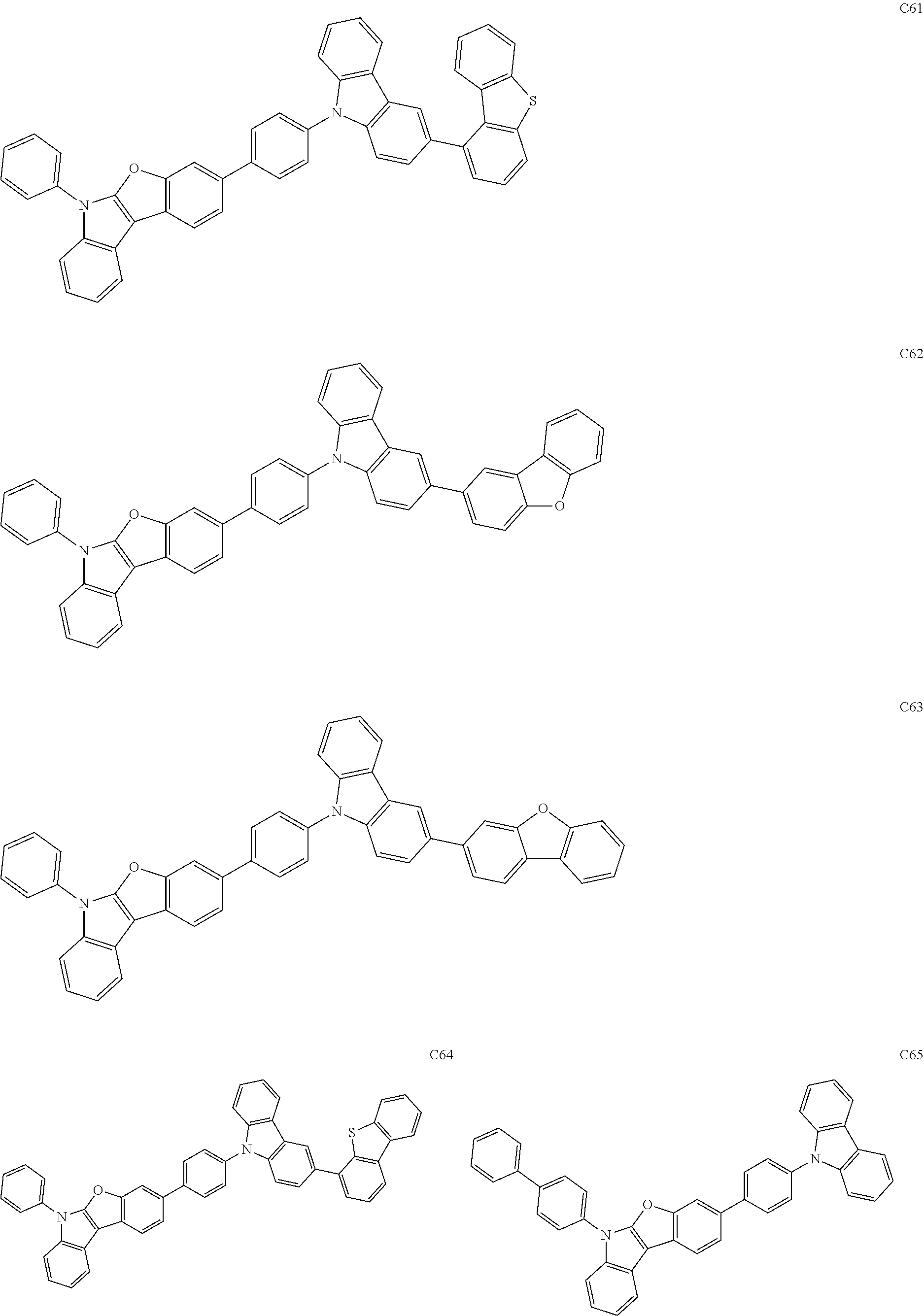 [SMILES: c1ccc(-n2c3ccccc3c3c4ccc(-c5ccc(-n6c7ccccc7c7cc(-c8cccc9sc%10ccccc%10c89)ccc76)cc5)cc4oc32)cc1 ; c1ccc(-n2c3ccccc3c3c4ccc(-c5ccc(-n6c7ccccc7c7cc(-c8ccc9oc%10ccccc%10c9c8)ccc76)cc5)cc4oc32)cc1 ; c1ccc(-n2c3ccccc3c3c4ccc(-c5ccc(-n6c7ccccc7c7cc(-c8ccc9c(c8)oc8ccccc89)ccc76)cc5)cc4oc32)cc1 ; c1ccc(-n2c3ccccc3c3c4ccc(-c5ccc(-n6c7ccccc7c7cc(-c8cccc9c8sc8ccccc89)ccc76)cc5)cc4oc32)cc1 ; c1ccc(-c2ccc(-n3c4ccccc4c4c5ccc(-c6ccc(-n7c8ccccc8c8ccccc87)cc6)cc5oc43)cc2)cc1]

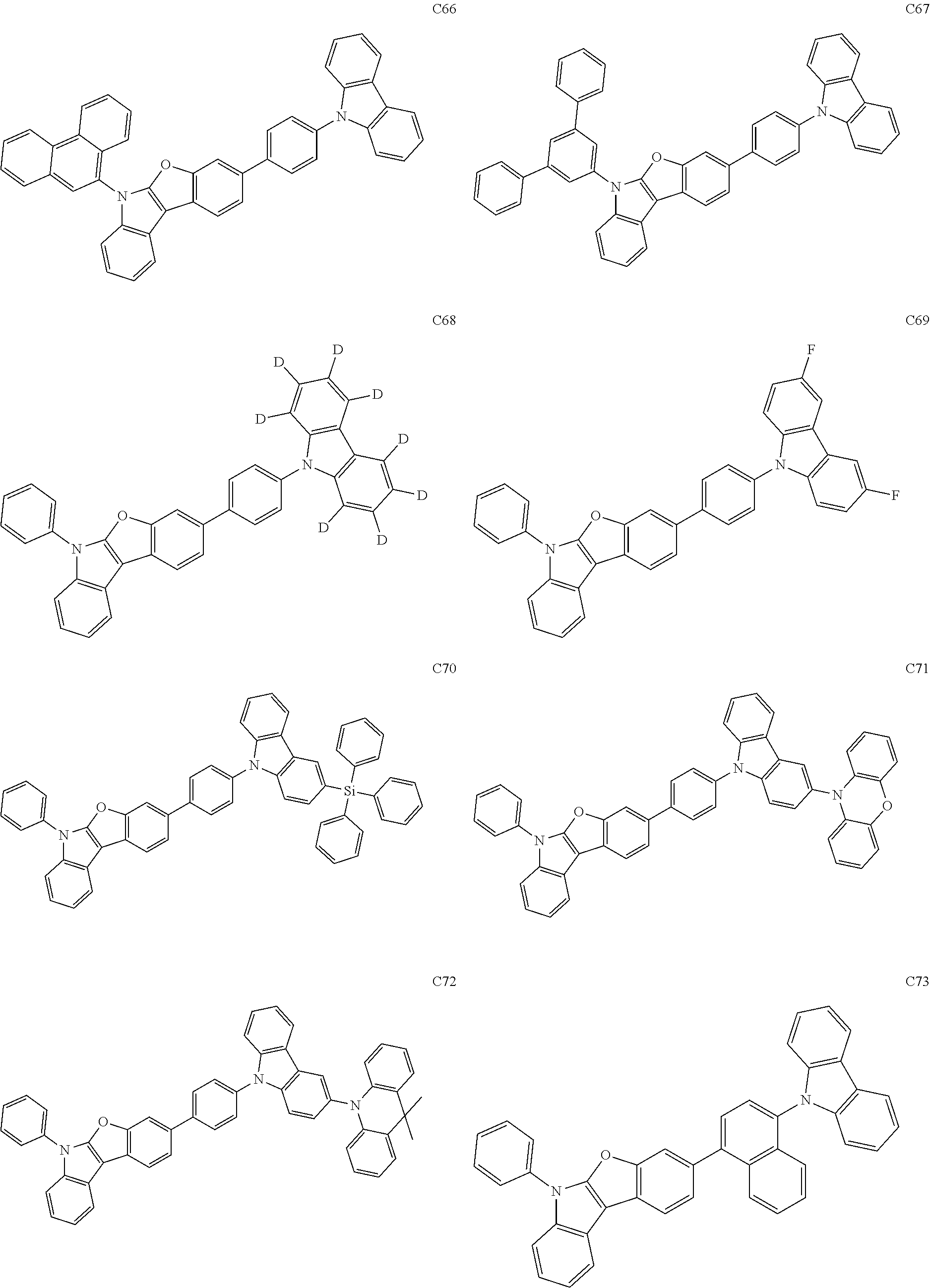

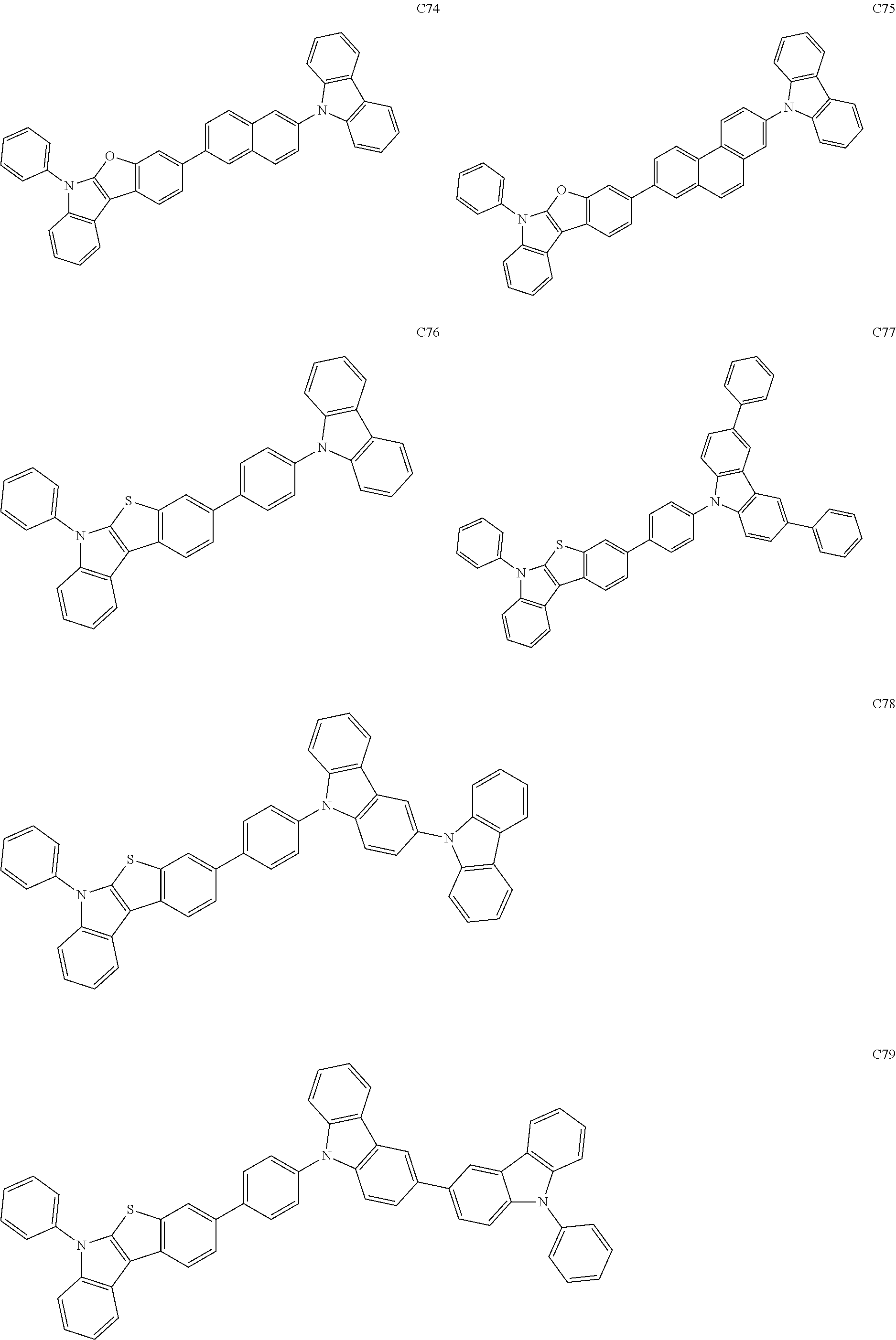

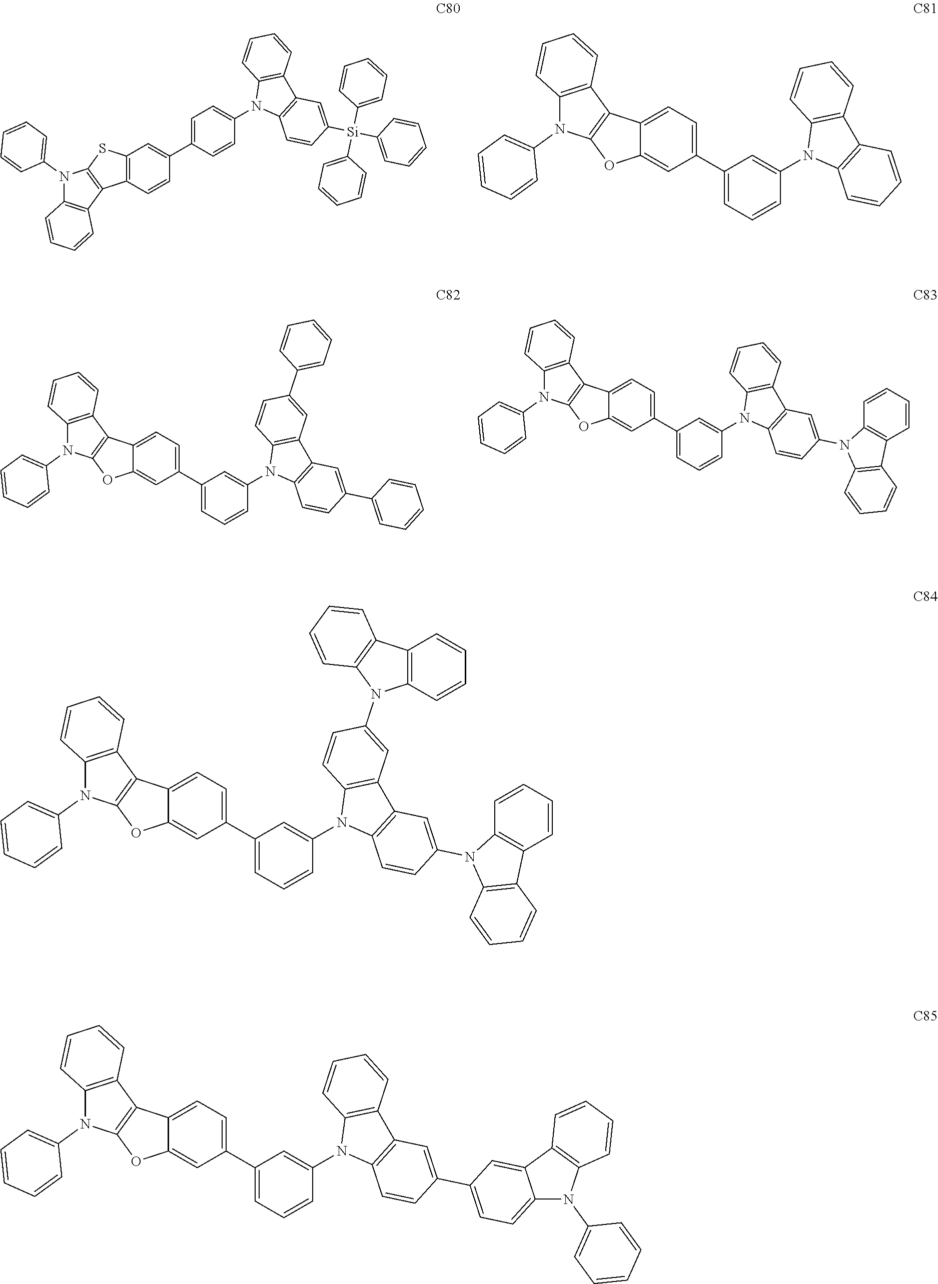

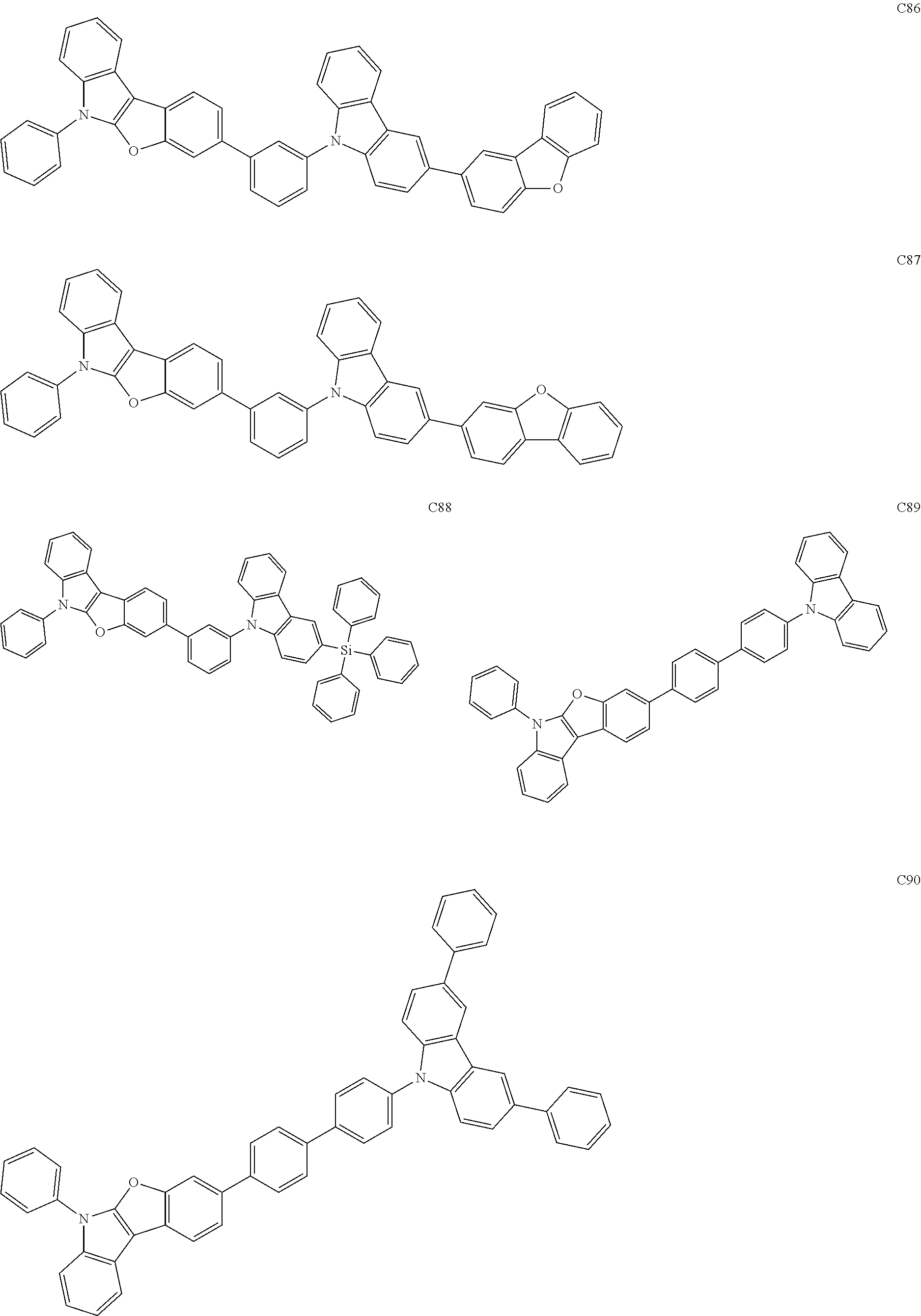 [SMILES: c1ccc(-n2c3ccccc3c3c4ccc(-c5cccc(-n6c7ccccc7c7cc(-c8ccc9oc%10ccccc%10c9c8)ccc76)c5)cc4oc32)cc1 ; c1ccc(-n2c3ccccc3c3c4ccc(-c5cccc(-n6c7ccccc7c7cc(-c8ccc9c(c8)oc8ccccc89)ccc76)c5)cc4oc32)cc1 ; c1ccc(-n2c3ccccc3c3c4ccc(-c5cccc(-n6c7ccccc7c7cc([Si](c8ccccc8)(c8ccccc8)c8ccccc8)ccc76)c5)cc4oc32)cc1 ; c1ccc(-n2c3ccccc3c3c4ccc(-c5ccc(-c6ccc(-n7c8ccccc8c8ccccc87)cc6)cc5)cc4oc32)cc1 ; c1ccc(-c2ccc3c(c2)c2cc(-c4ccccc4)ccc2n3-c2ccc(-c3ccc(-c4ccc5c(c4)oc4c5c5ccccc5n4-c4ccccc4)cc3)cc2)cc1]

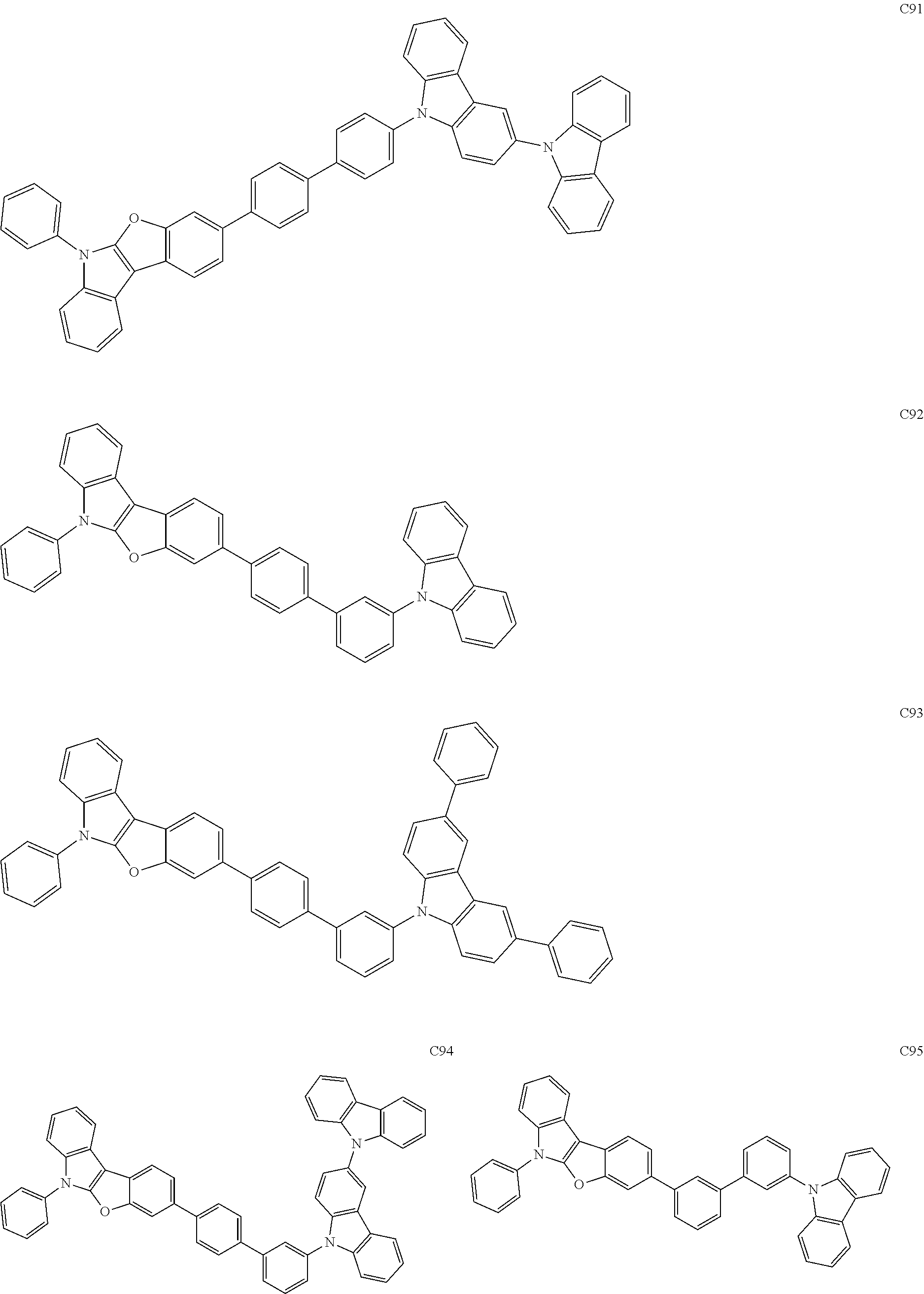 [SMILES: c1ccc(-n2c3ccccc3c3c4ccc(-c5ccc(-c6ccc(-n7c8ccccc8c8cc(-n9c%10ccccc%10c%10ccccc%109)ccc87)cc6)cc5)cc4oc32)cc1 ; c1ccc(-n2c3ccccc3c3c4ccc(-c5ccc(-c6cccc(-n7c8ccccc8c8ccccc87)c6)cc5)cc4oc32)cc1 ; c1ccc(-c2ccc3c(c2)c2cc(-c4ccccc4)ccc2n3-c2cccc(-c3ccc(-c4ccc5c(c4)oc4c5c5ccccc5n4-c4ccccc4)cc3)c2)cc1 ; c1ccc(-n2c3ccccc3c3c4ccc(-c5ccc(-c6cccc(-n7c8ccccc8c8cc(-n9c%10ccccc%10c%10ccccc%109)ccc87)c6)cc5)cc4oc32)cc1 ; c1ccc(-n2c3ccccc3c3c4ccc(-c5cccc(-c6cccc(-n7c8ccccc8c8ccccc87)c6)c5)cc4oc32)cc1]

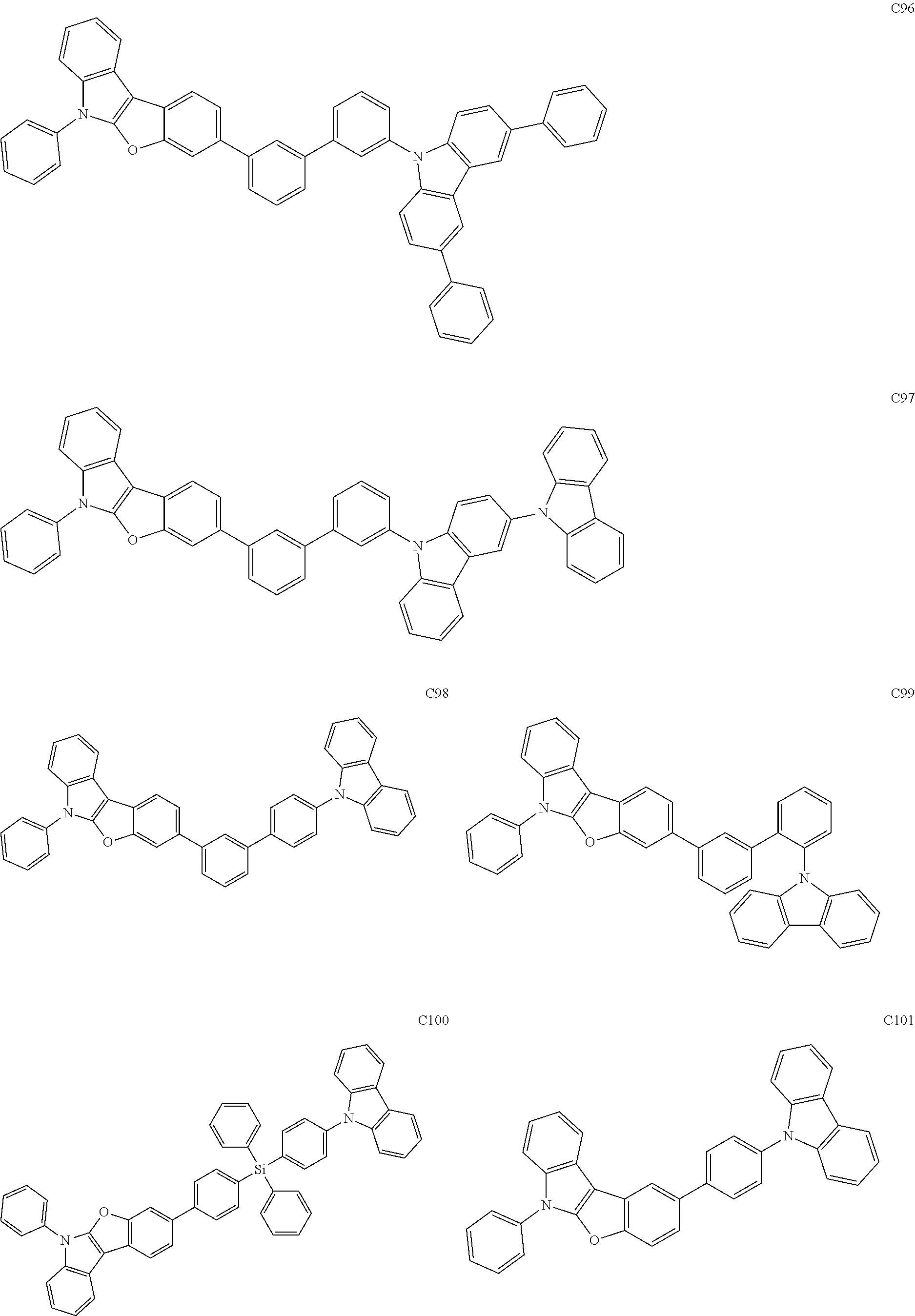

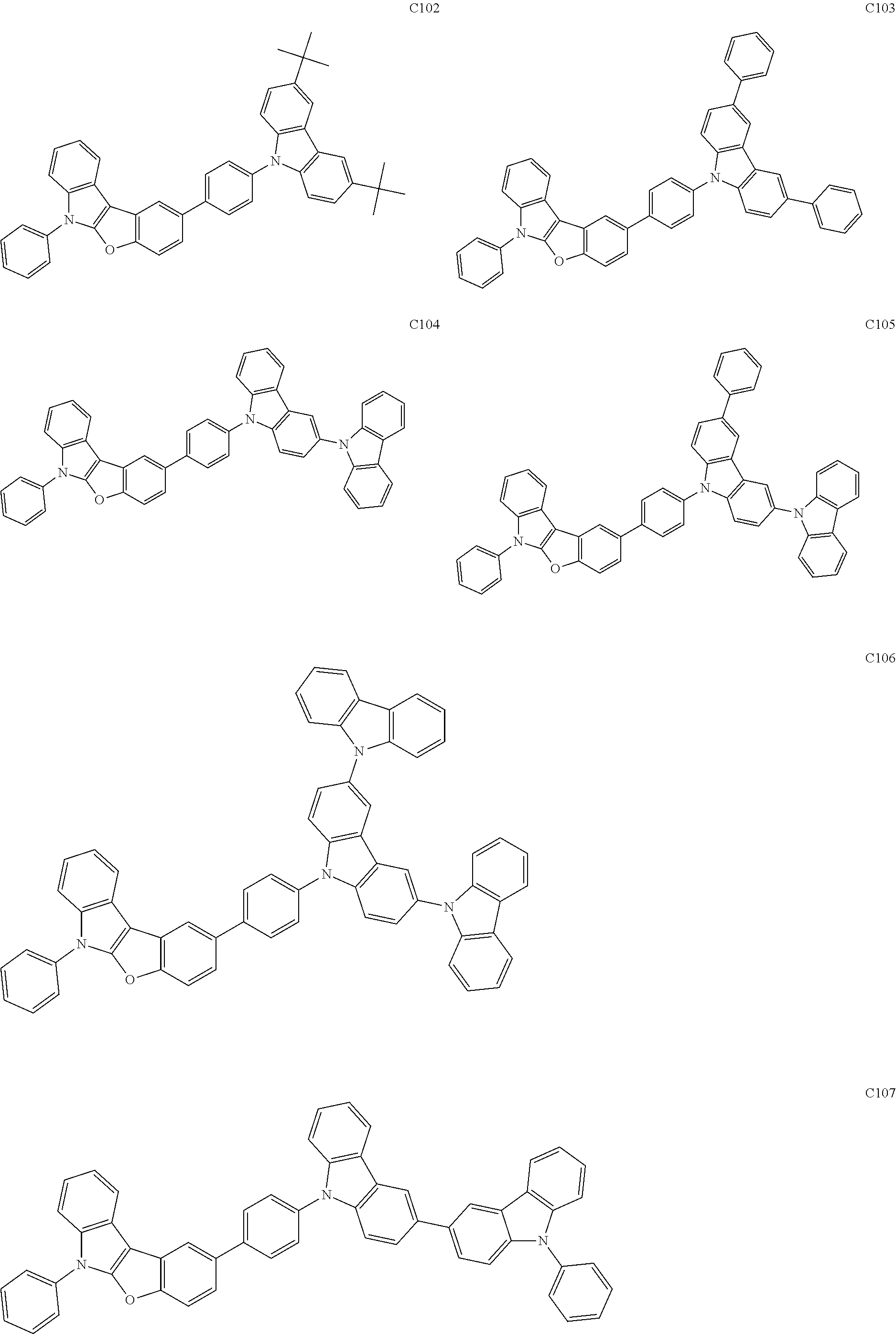

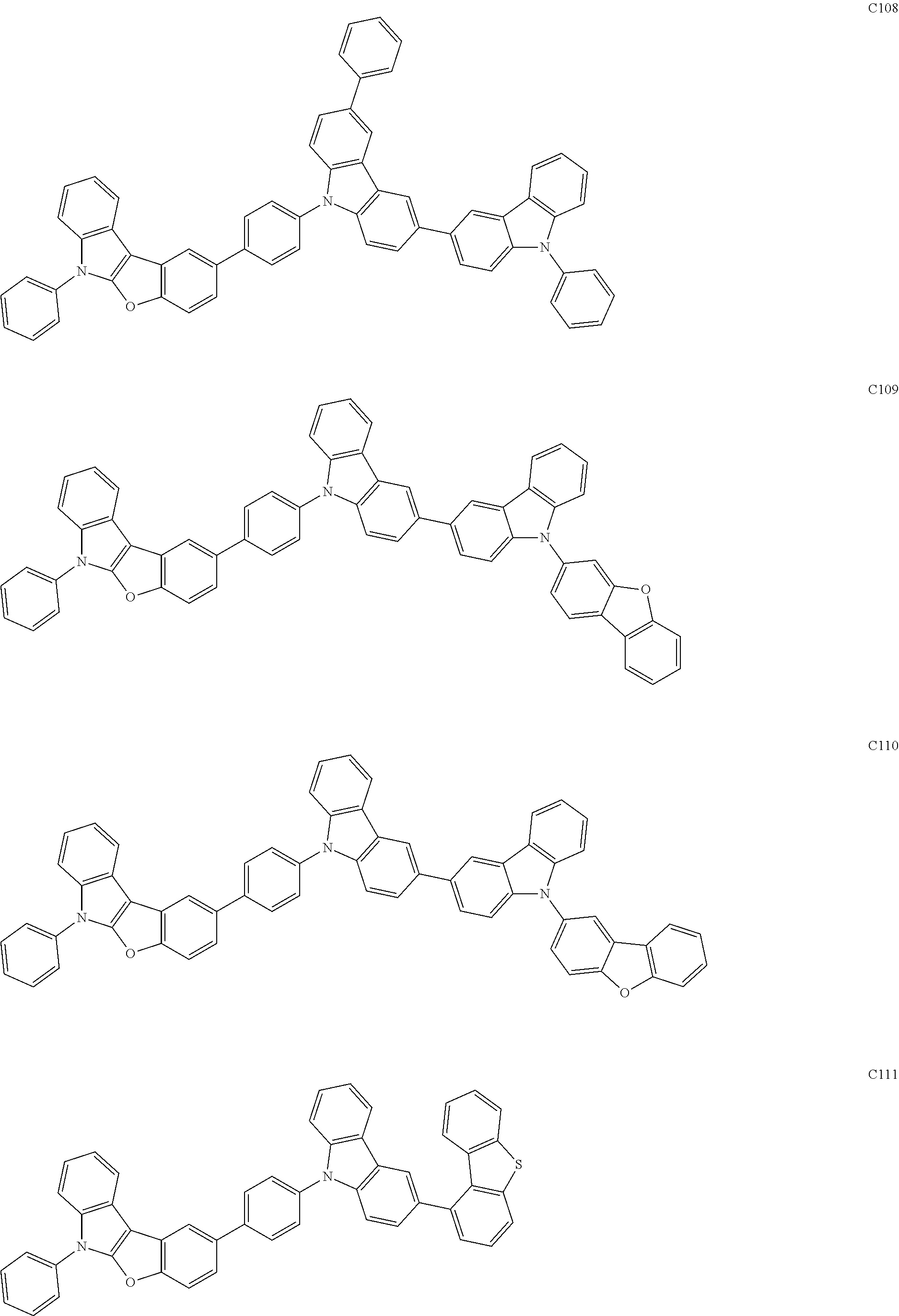 [SMILES: c1ccc(-c2ccc3c(c2)c2cc(-c4ccc5c(c4)c4ccccc4n5-c4ccccc4)ccc2n3-c2ccc(-c3ccc4oc5c(c4c3)c3ccccc3n5-c3ccccc3)cc2)cc1 ; c1ccc(-n2c3ccccc3c3c4cc(-c5ccc(-n6c7ccccc7c7cc(-c8ccc9c(c8)c8ccccc8n9-c8ccc9c(c8)oc8ccccc89)ccc76)cc5)ccc4oc32)cc1 ; c1ccc(-n2c3ccccc3c3c4cc(-c5ccc(-n6c7ccccc7c7cc(-c8ccc9c(c8)c8ccccc8n9-c8ccc9oc%10ccccc%10c9c8)ccc76)cc5)ccc4oc32)cc1 ; c1ccc(-n2c3ccccc3c3c4cc(-c5ccc(-n6c7ccccc7c7cc(-c8cccc9sc%10ccccc%10c89)ccc76)cc5)ccc4oc32)cc1]

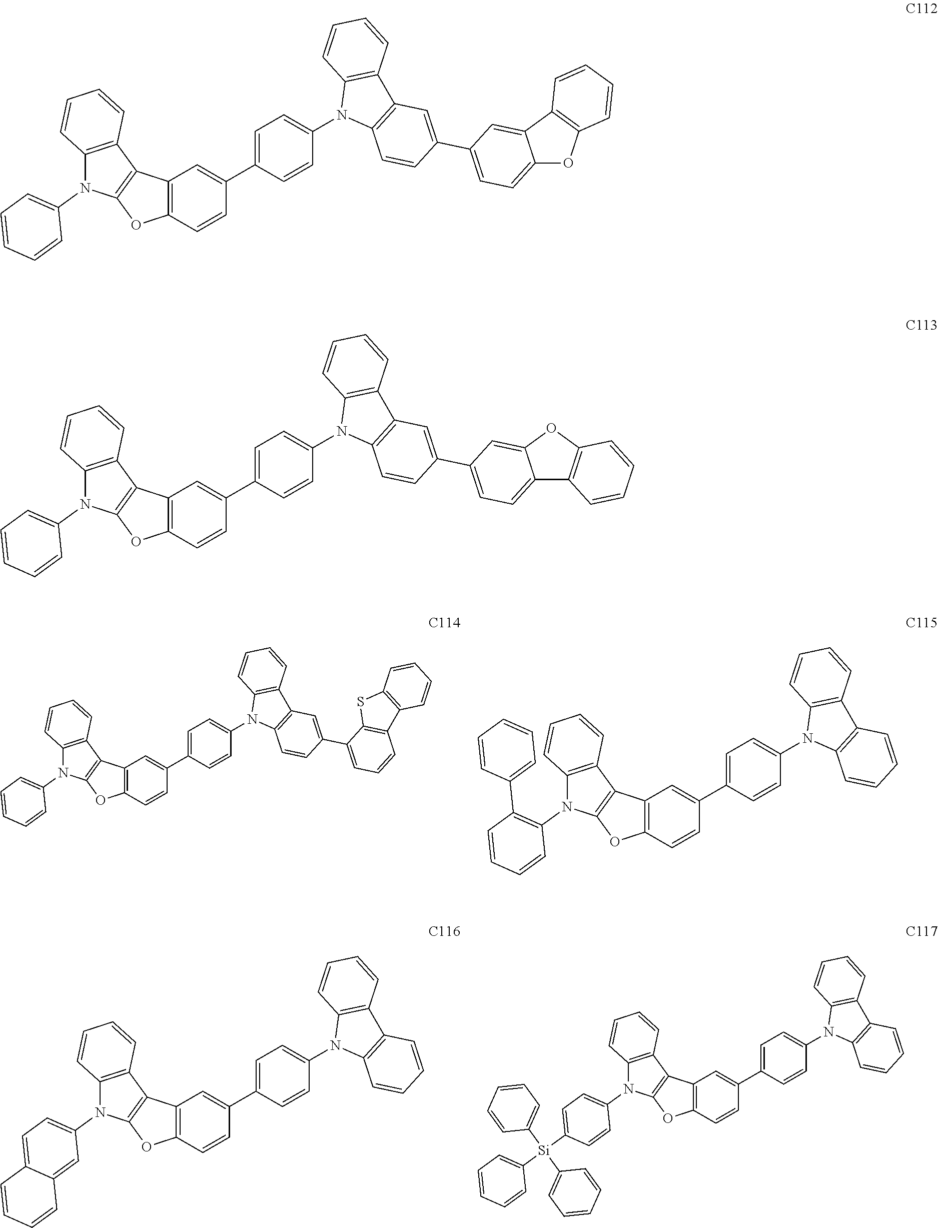

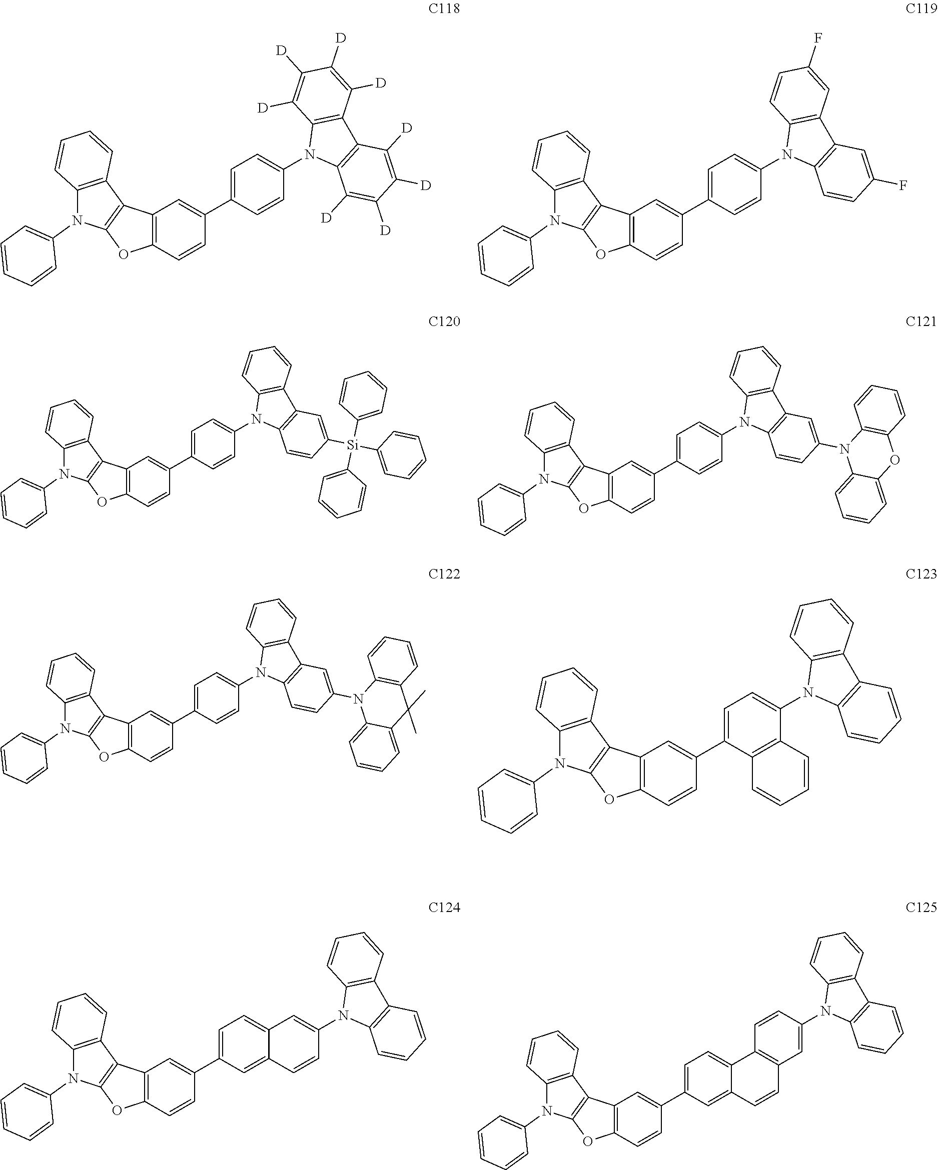

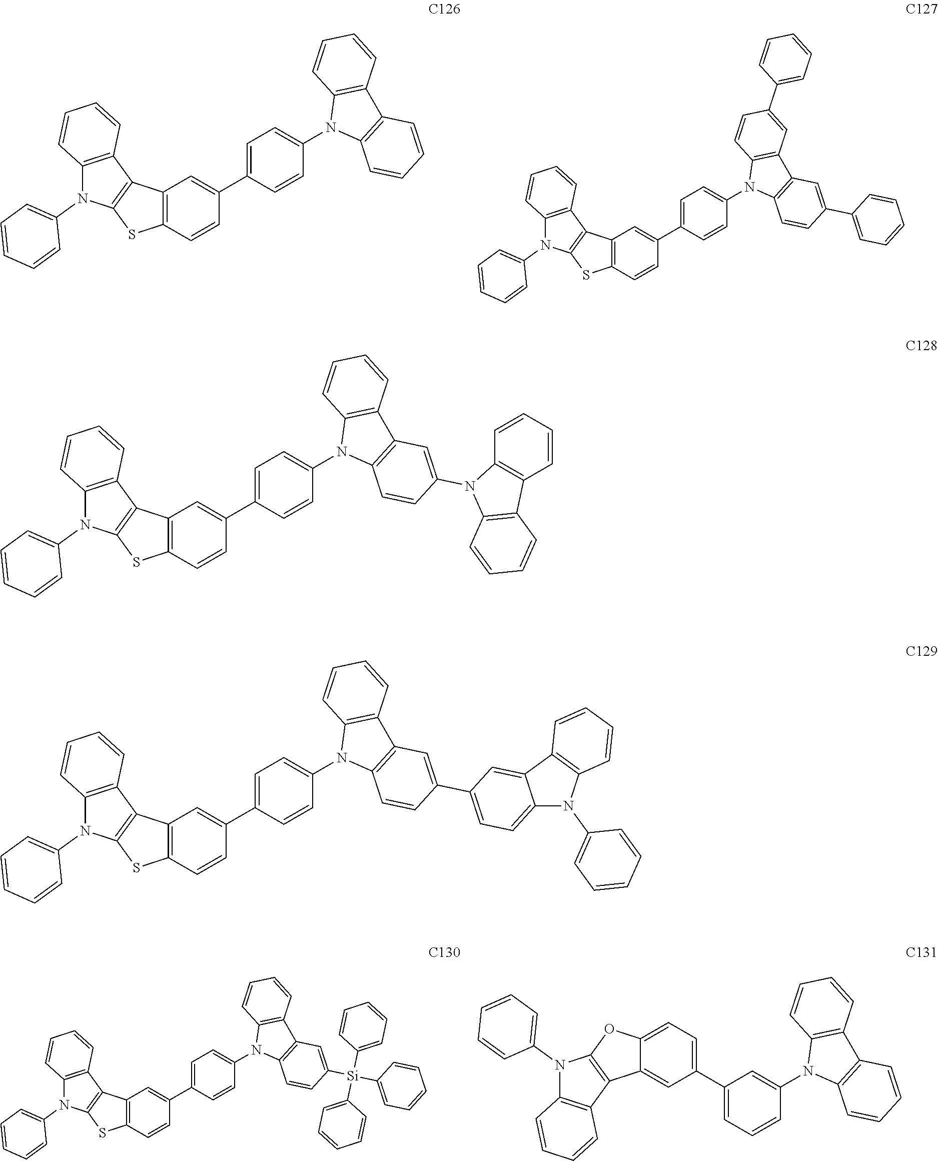

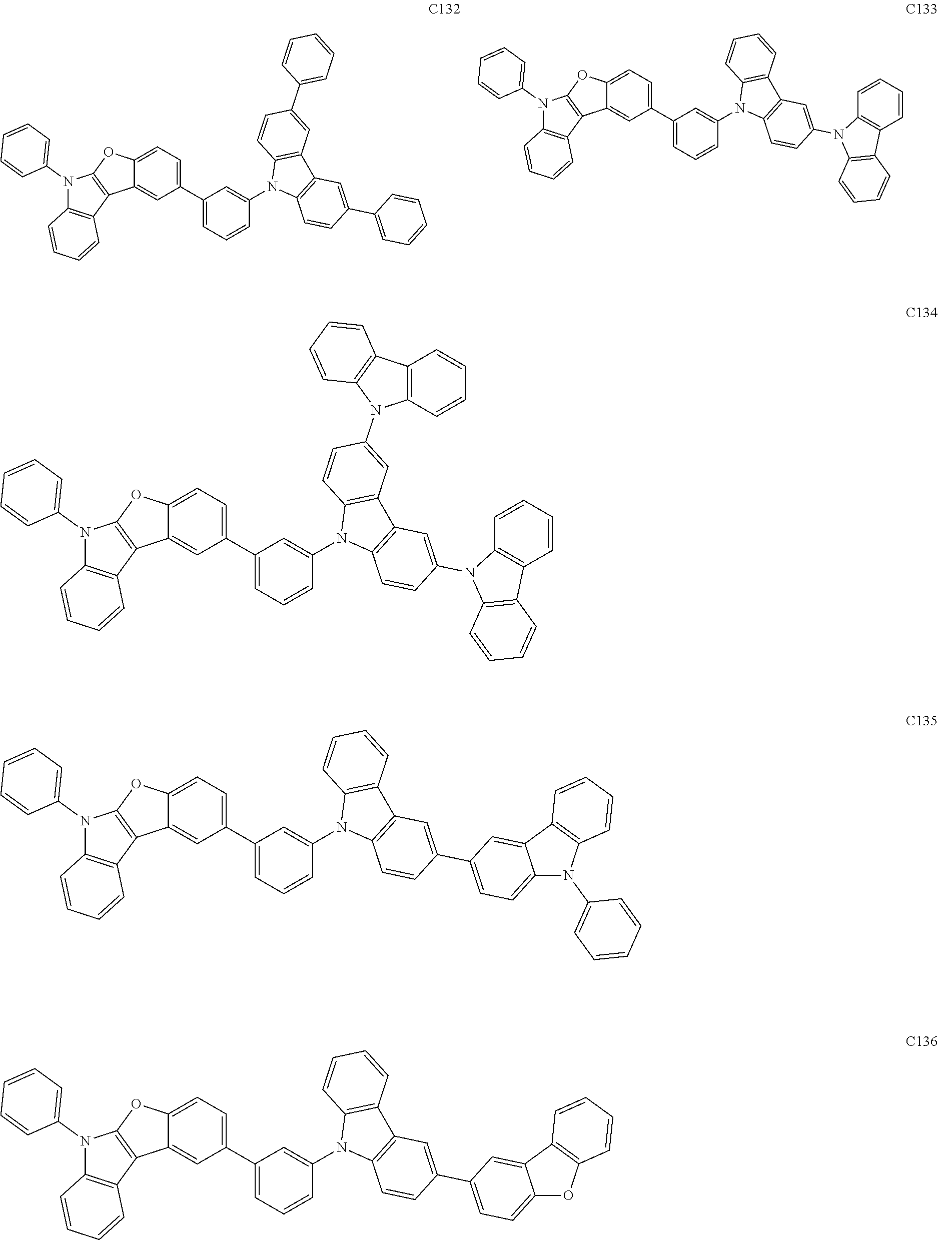 [SMILES: c1ccc(-c2ccc3c(c2)c2cc(-c4ccccc4)ccc2n3-c2cccc(-c3ccc4oc5c(c4c3)c3ccccc3n5-c3ccccc3)c2)cc1 ; c1ccc(-n2c3ccccc3c3c4cc(-c5cccc(-n6c7ccccc7c7cc(-n8c9ccccc9c9ccccc98)ccc76)c5)ccc4oc32)cc1 ; c1ccc(-n2c3ccccc3c3c4cc(-c5cccc(-n6c7ccc(-n8c9ccccc9c9ccccc98)cc7c7cc(-n8c9ccccc9c9ccccc98)ccc76)c5)ccc4oc32)cc1 ; c1ccc(-n2c3ccccc3c3cc(-c4ccc5c(c4)c4ccccc4n5-c4cccc(-c5ccc6oc7c(c6c5)c5ccccc5n7-c5ccccc5)c4)ccc32)cc1 ; c1ccc(-n2c3ccccc3c3c4cc(-c5cccc(-n6c7ccccc7c7cc(-c8ccc9oc%10ccccc%10c9c8)ccc76)c5)ccc4oc32)cc1]

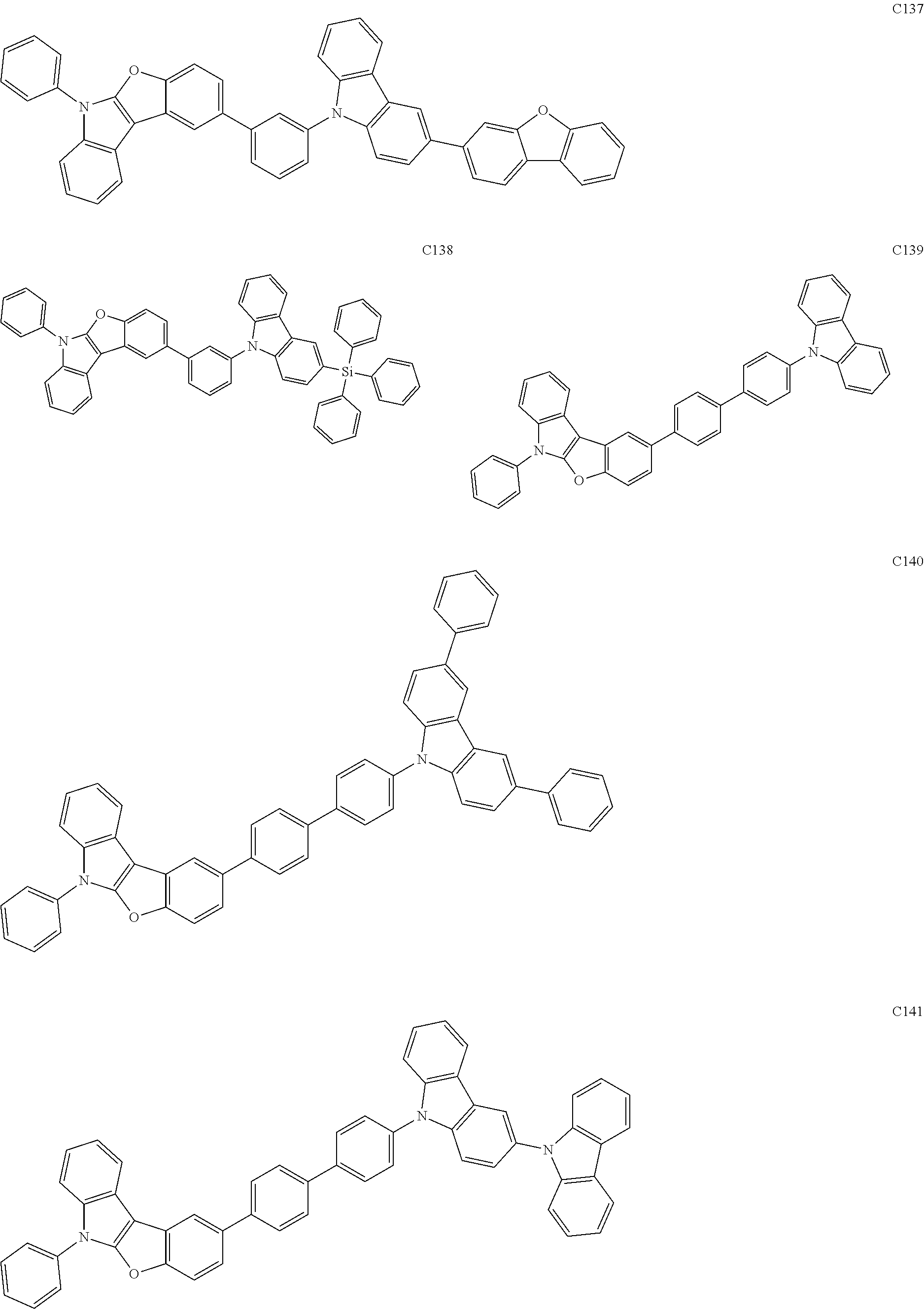 [SMILES: c1ccc(-n2c3ccccc3c3c4cc(-c5cccc(-n6c7ccccc7c7cc(-c8ccc9c(c8)oc8ccccc89)ccc76)c5)ccc4oc32)cc1 ; c1ccc(-n2c3ccccc3c3c4cc(-c5cccc(-n6c7ccccc7c7cc([Si](c8ccccc8)(c8ccccc8)c8ccccc8)ccc76)c5)ccc4oc32)cc1 ; c1ccc(-n2c3ccccc3c3c4cc(-c5ccc(-c6ccc(-n7c8ccccc8c8ccccc87)cc6)cc5)ccc4oc32)cc1 ; c1ccc(-c2ccc3c(c2)c2cc(-c4ccccc4)ccc2n3-c2ccc(-c3ccc(-c4ccc5oc6c(c5c4)c4ccccc4n6-c4ccccc4)cc3)cc2)cc1 ; c1ccc(-n2c3ccccc3c3c4cc(-c5ccc(-c6ccc(-n7c8ccccc8c8cc(-n9c%10ccccc%10c%10ccccc%109)ccc87)cc6)cc5)ccc4oc32)cc1]

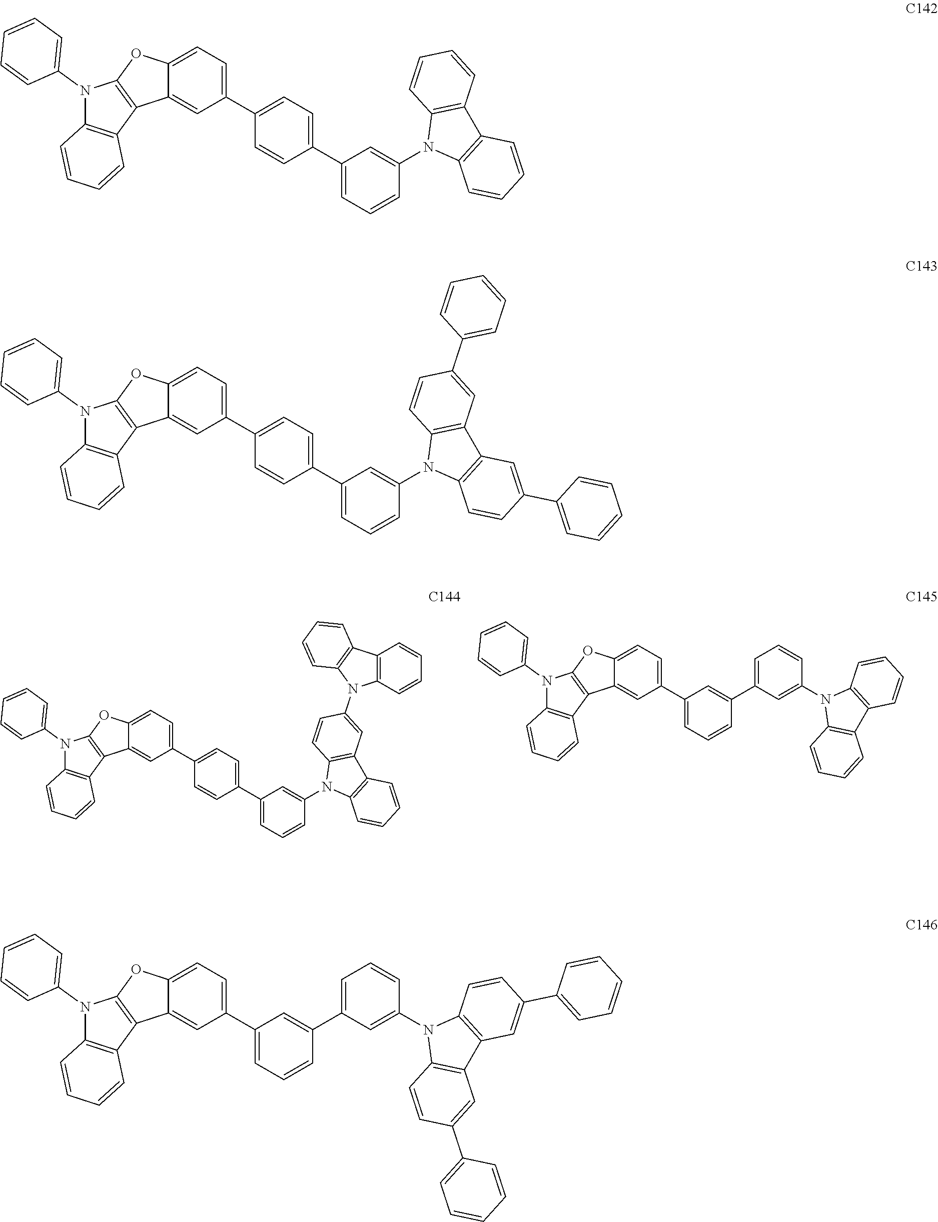 [SMILES: c1ccc(-n2c3ccccc3c3c4cc(-c5ccc(-c6cccc(-n7c8ccccc8c8ccccc87)c6)cc5)ccc4oc32)cc1 ; c1ccc(-c2ccc3c(c2)c2cc(-c4ccccc4)ccc2n3-c2cccc(-c3ccc(-c4ccc5oc6c(c5c4)c4ccccc4n6-c4ccccc4)cc3)c2)cc1 ; c1ccc(-n2c3ccccc3c3c4cc(-c5ccc(-c6cccc(-n7c8ccccc8c8cc(-n9c%10ccccc%10c%10ccccc%109)ccc87)c6)cc5)ccc4oc32)cc1 ; c1ccc(-n2c3ccccc3c3c4cc(-c5cccc(-c6cccc(-n7c8ccccc8c8ccccc87)c6)c5)ccc4oc32)cc1 ; c1ccc(-c2ccc3c(c2)c2cc(-c4ccccc4)ccc2n3-c2cccc(-c3cccc(-c4ccc5oc6c(c5c4)c4ccccc4n6-c4ccccc4)c3)c2)cc1]

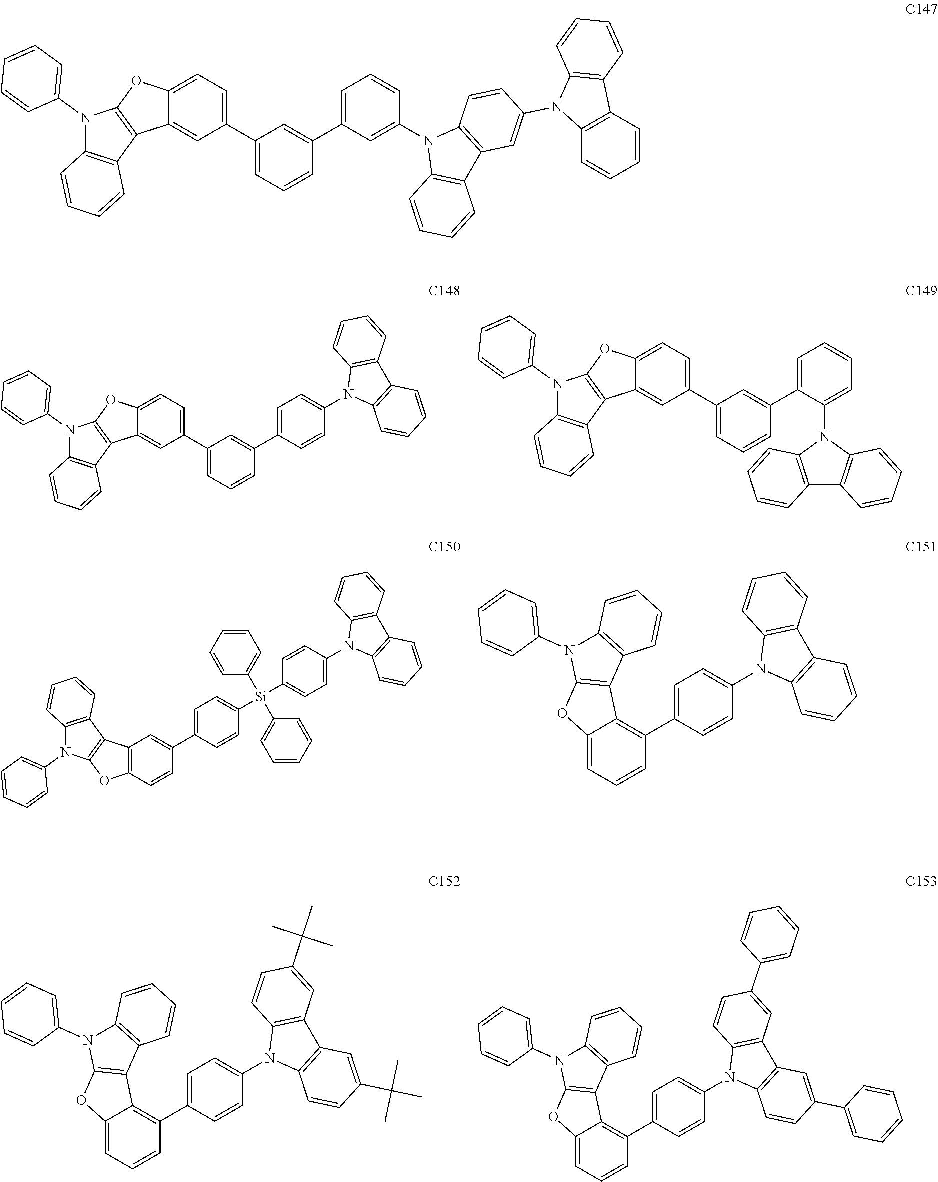

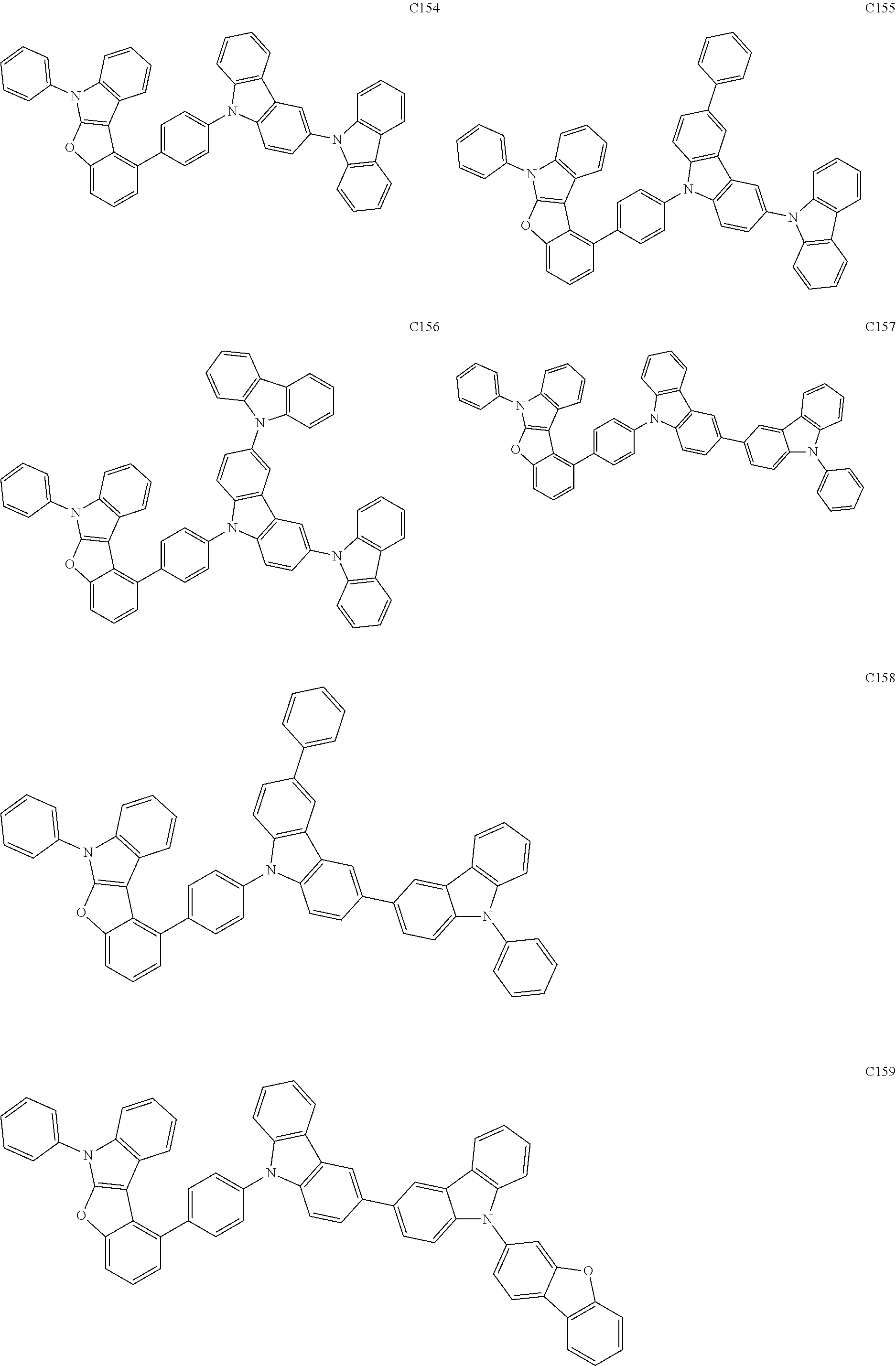

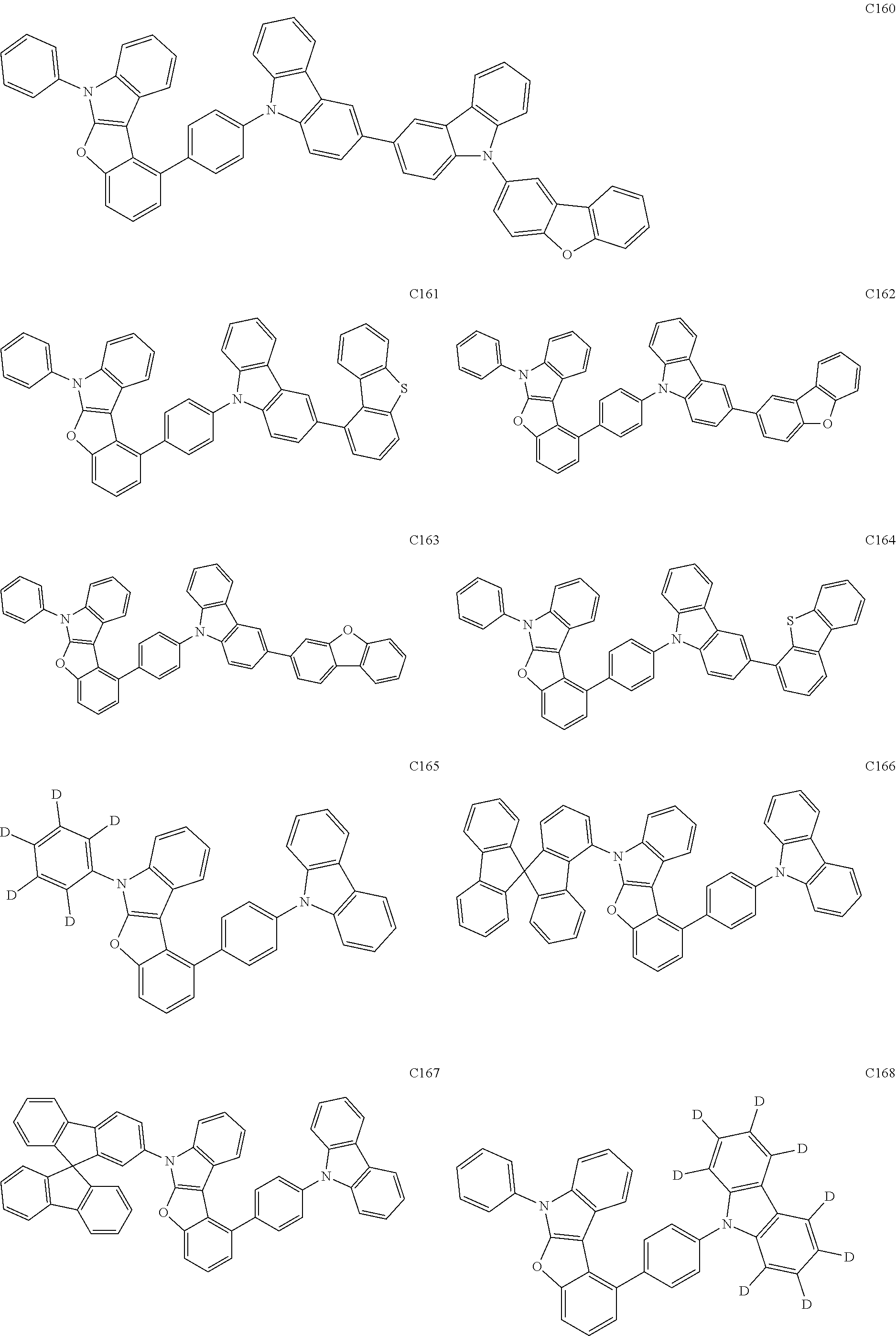

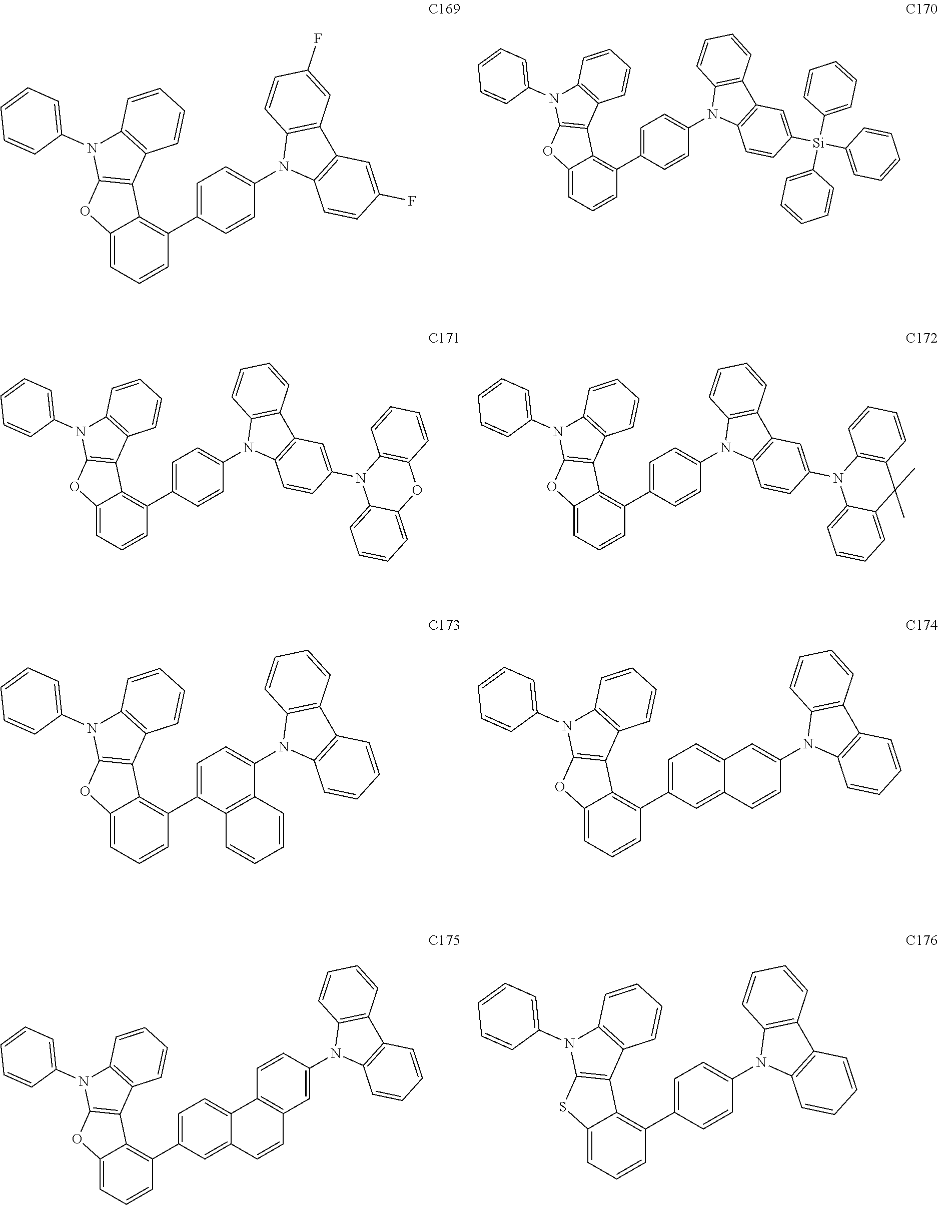

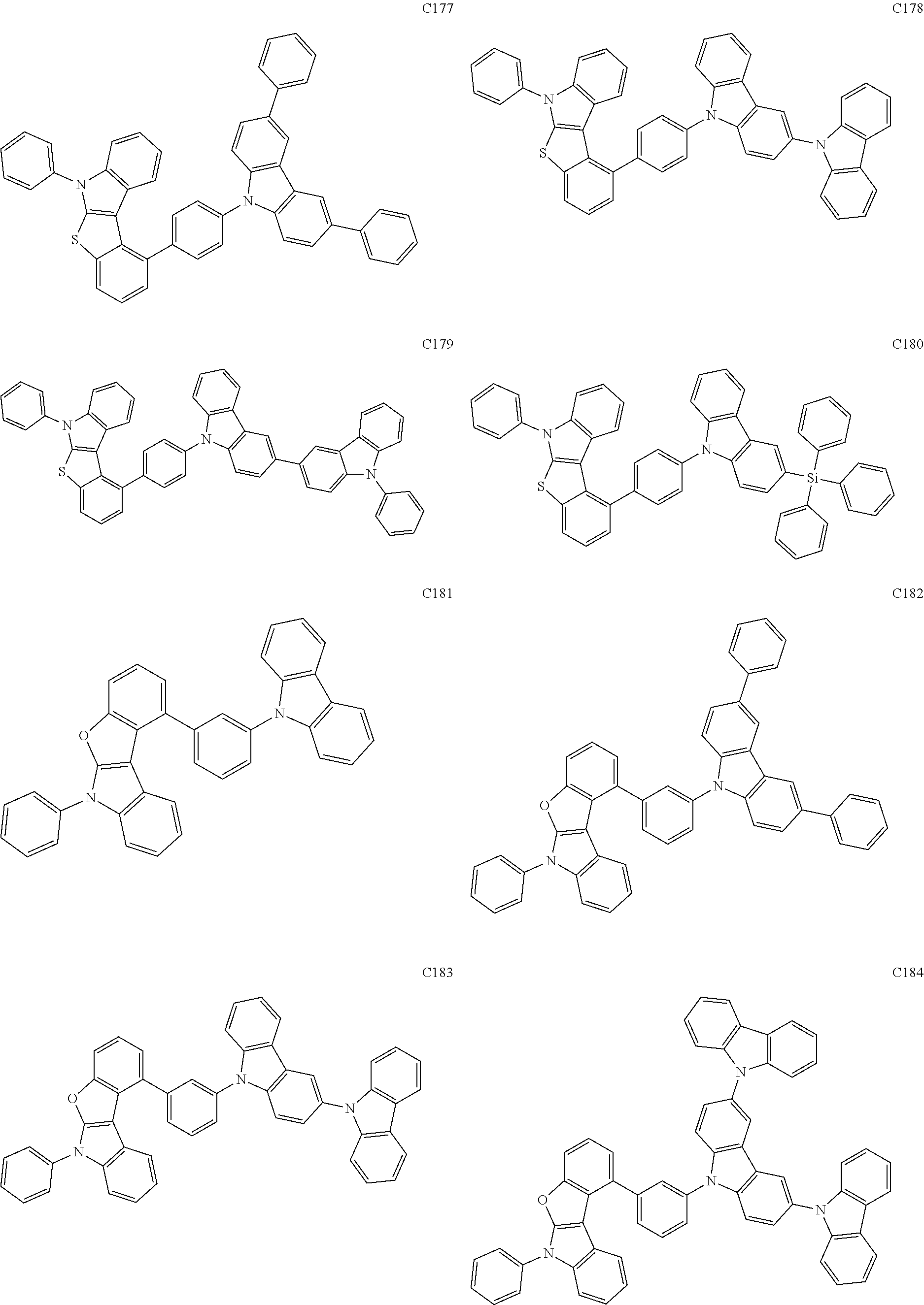

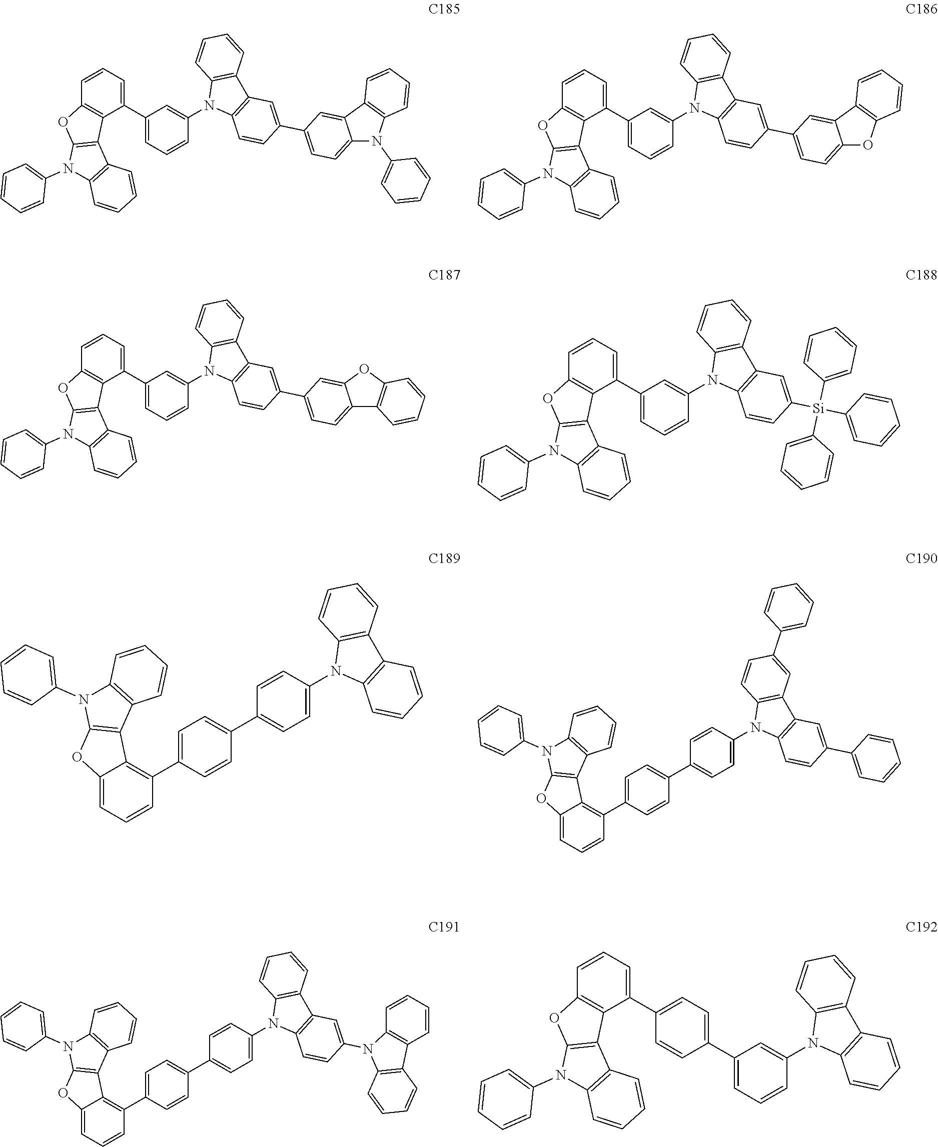

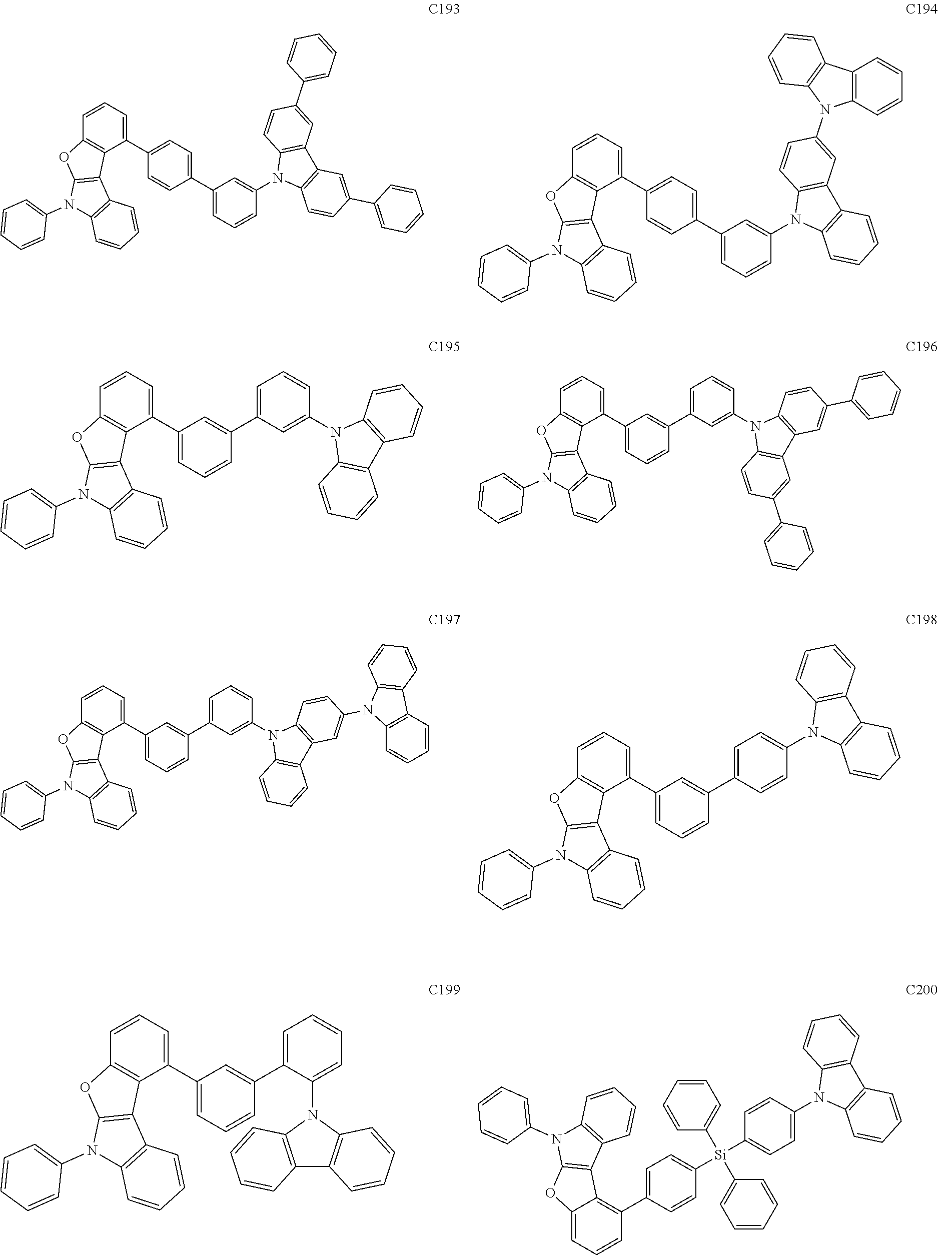

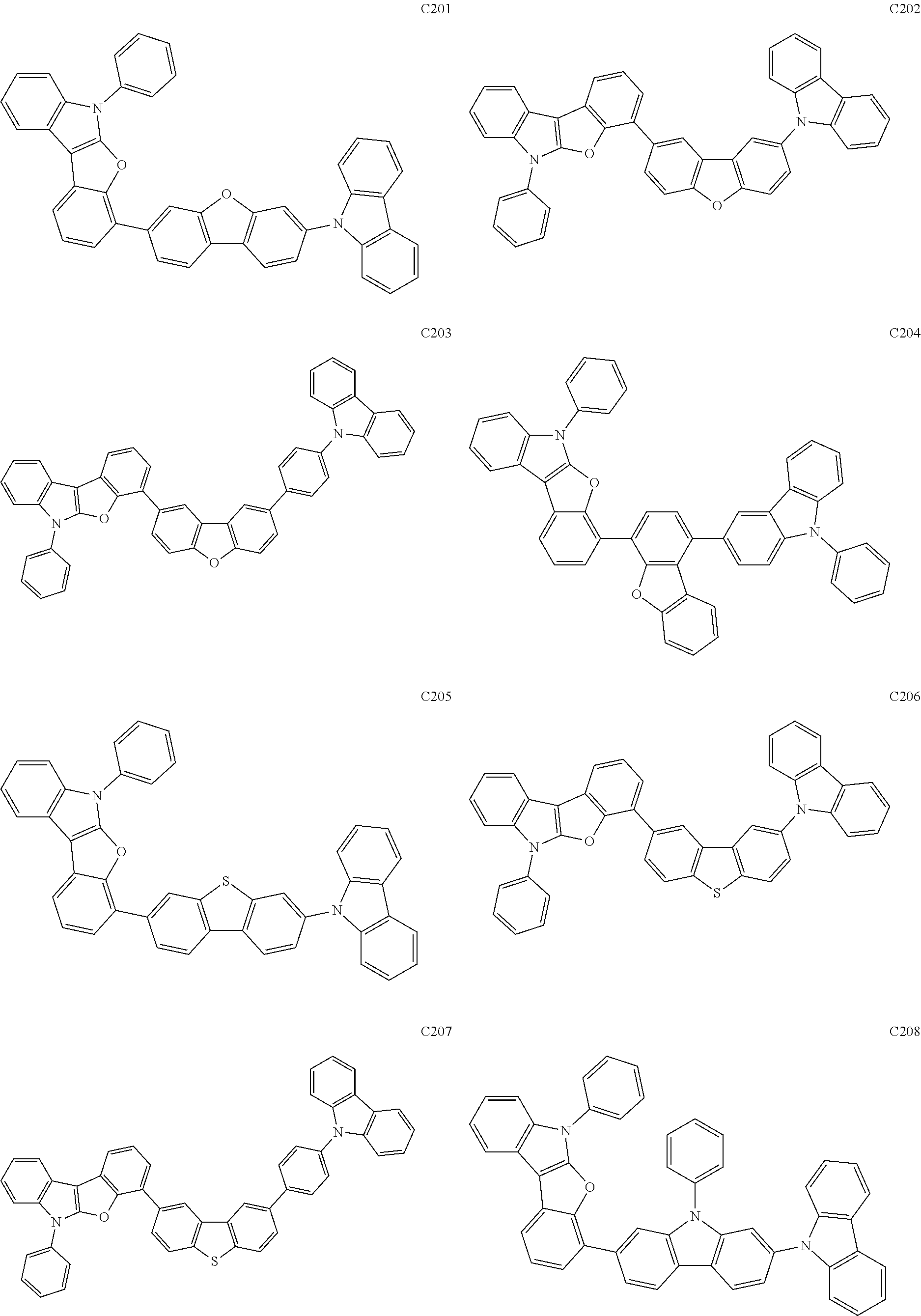

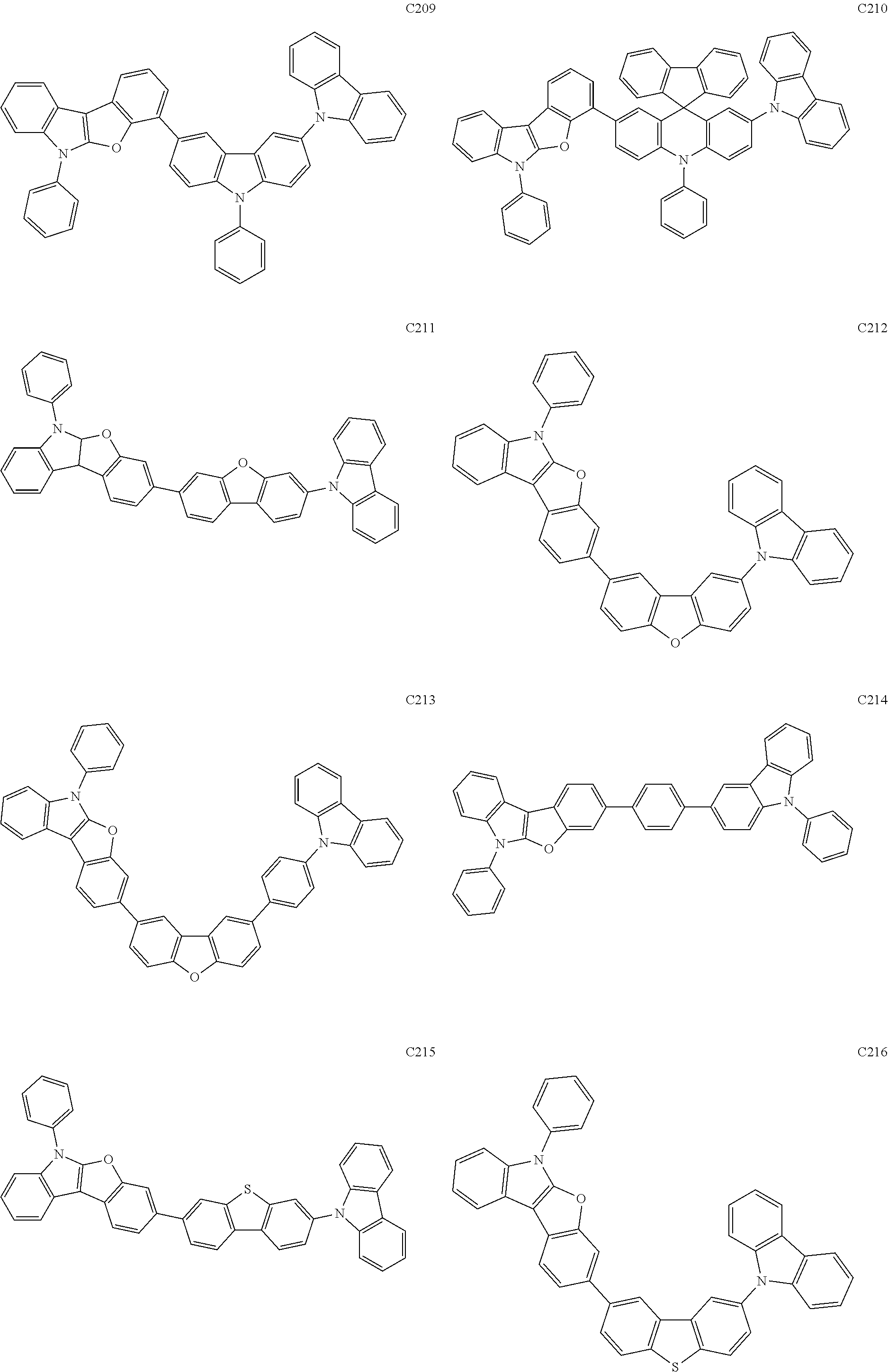

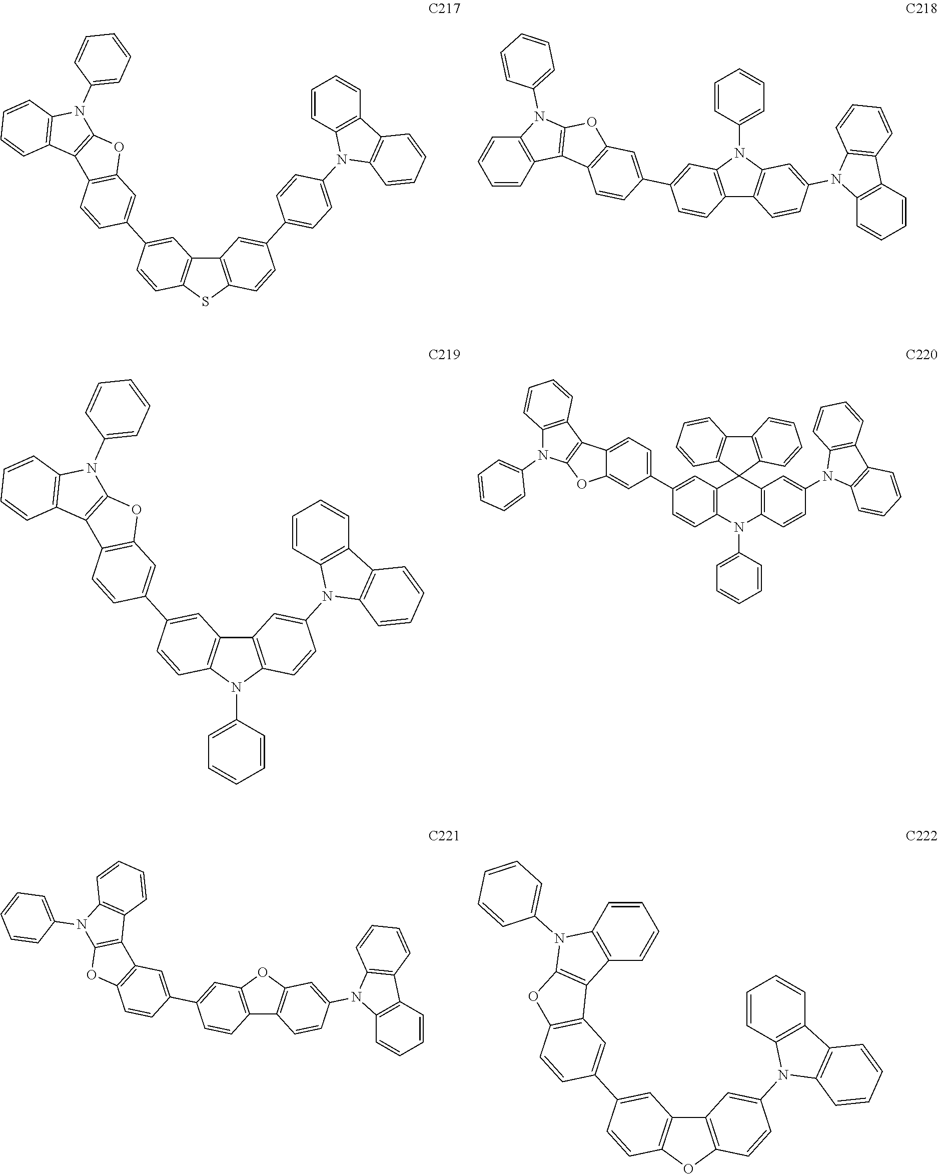

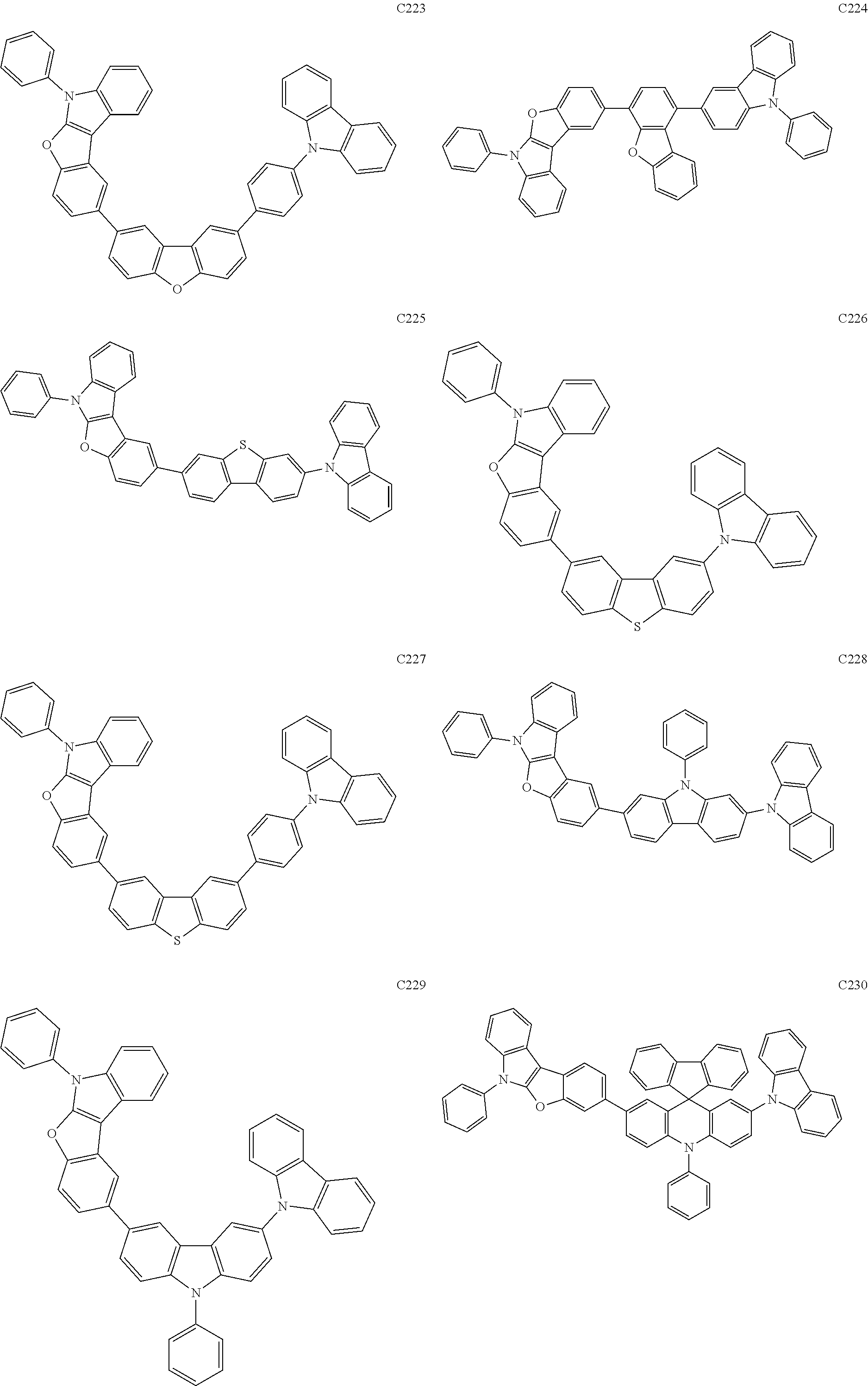

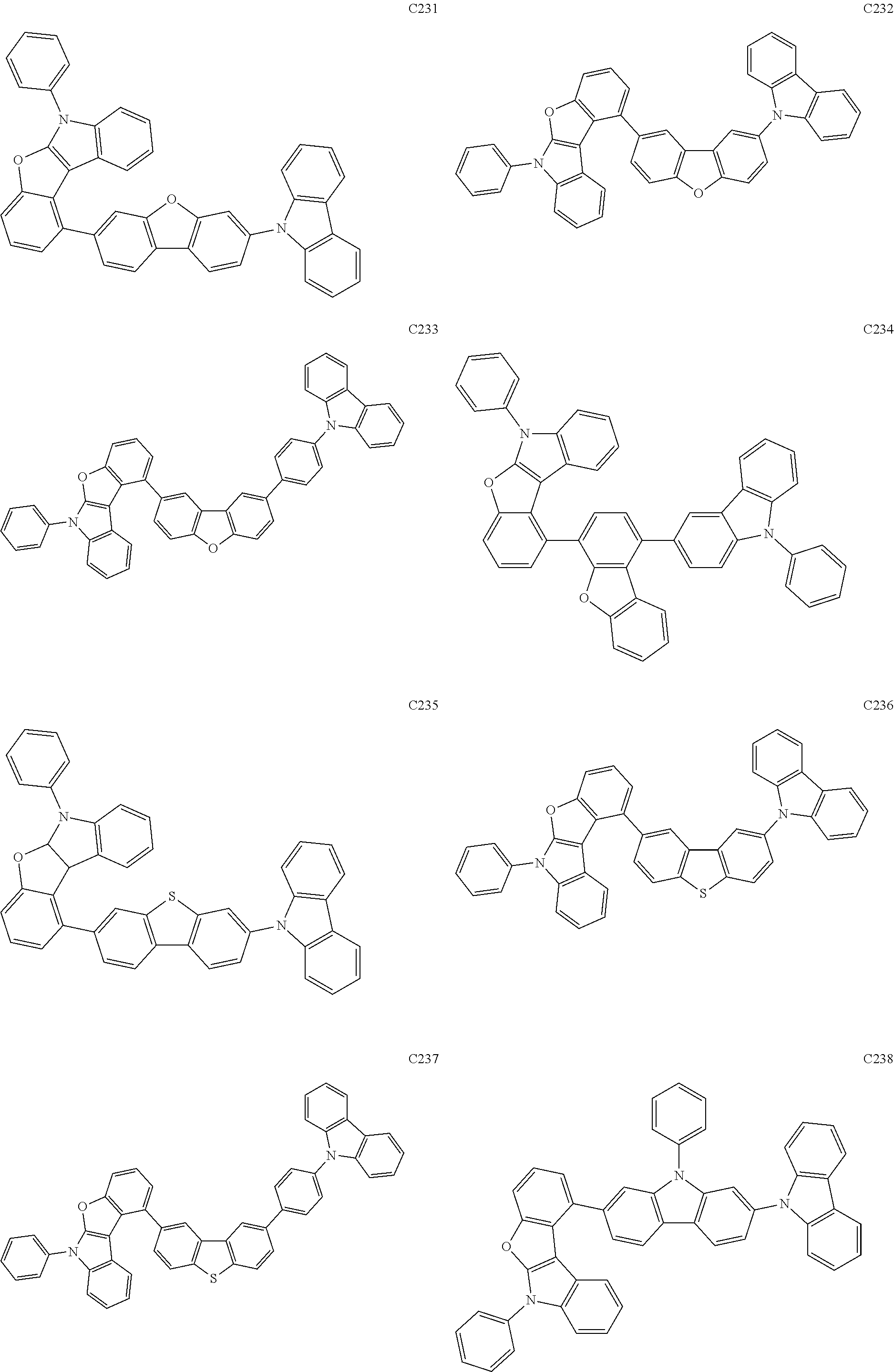

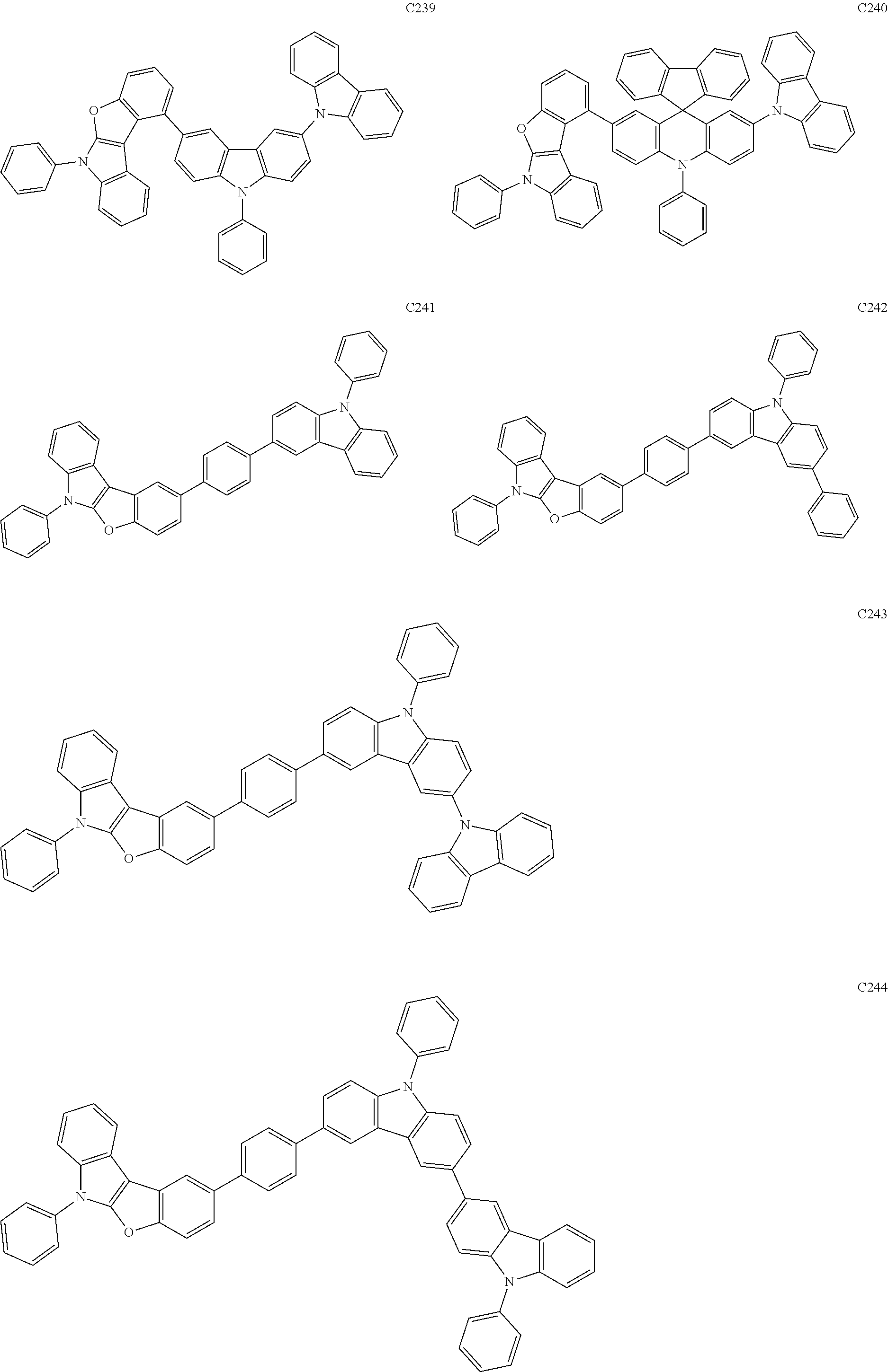

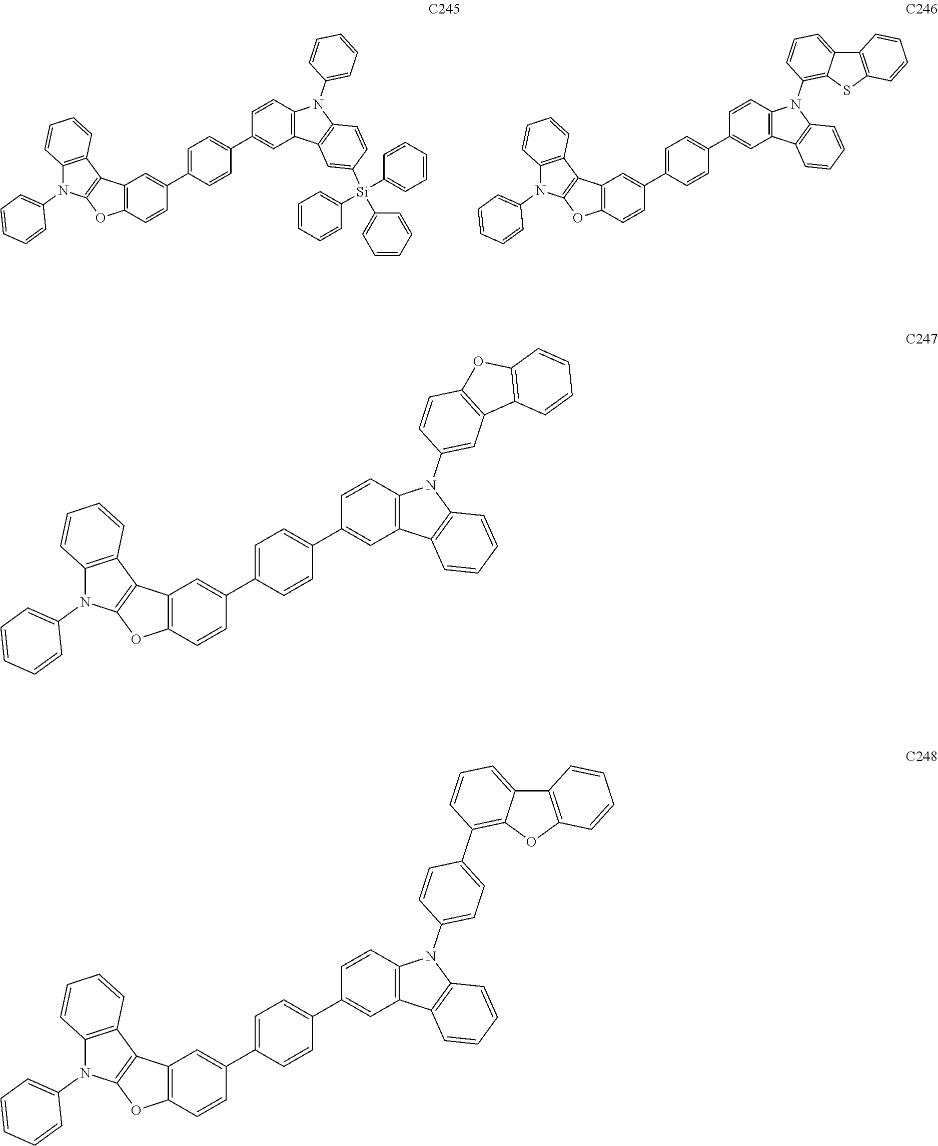 [SMILES: c1ccc(-n2c3ccc(-c4ccc(-c5ccc6oc7c(c6c5)c5ccccc5n7-c5ccccc5)cc4)cc3c3cc([Si](c4ccccc4)(c4ccccc4)c4ccccc4)ccc32)cc1 ; c1ccc(-n2c3ccccc3c3c4cc(-c5ccc(-c6ccc7c(c6)c6ccccc6n7-c6cccc7c6sc6ccccc67)cc5)ccc4oc32)cc1 ; c1ccc(-n2c3ccccc3c3c4cc(-c5ccc(-c6ccc7c(c6)c6ccccc6n7-c6ccc7oc8ccccc8c7c6)cc5)ccc4oc32)cc1 ; c1ccc(-n2c3ccccc3c3c4cc(-c5ccc(-c6ccc7c(c6)c6ccccc6n7-c6ccc(-c7cccc8c7oc7ccccc78)cc6)cc5)ccc4oc32)cc1]

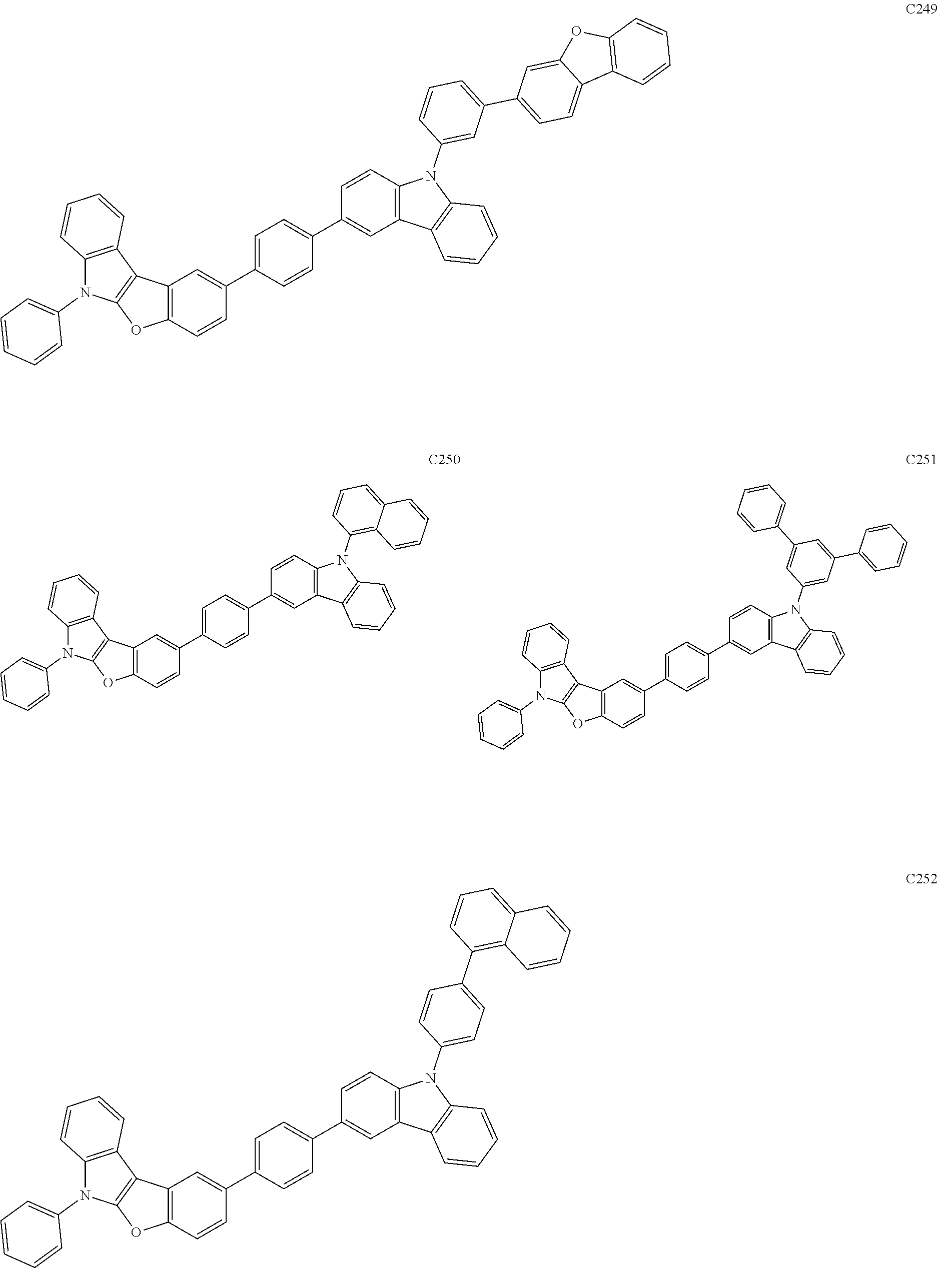 [SMILES: c1ccc(-n2c3ccccc3c3c4cc(-c5ccc(-c6ccc7c(c6)c6ccccc6n7-c6cccc(-c7ccc8c(c7)oc7ccccc78)c6)cc5)ccc4oc32)cc1 ; c1ccc(-n2c3ccccc3c3c4cc(-c5ccc(-c6ccc7c(c6)c6ccccc6n7-c6cccc7ccccc67)cc5)ccc4oc32)cc1 ; c1ccc(-c2cc(-c3ccccc3)cc(-n3c4ccccc4c4cc(-c5ccc(-c6ccc7oc8c(c7c6)c6ccccc6n8-c6ccccc6)cc5)ccc43)c2)cc1 ; c1ccc(-n2c3ccccc3c3c4cc(-c5ccc(-c6ccc7c(c6)c6ccccc6n7-c6ccc(-c7cccc8ccccc78)cc6)cc5)ccc4oc32)cc1]

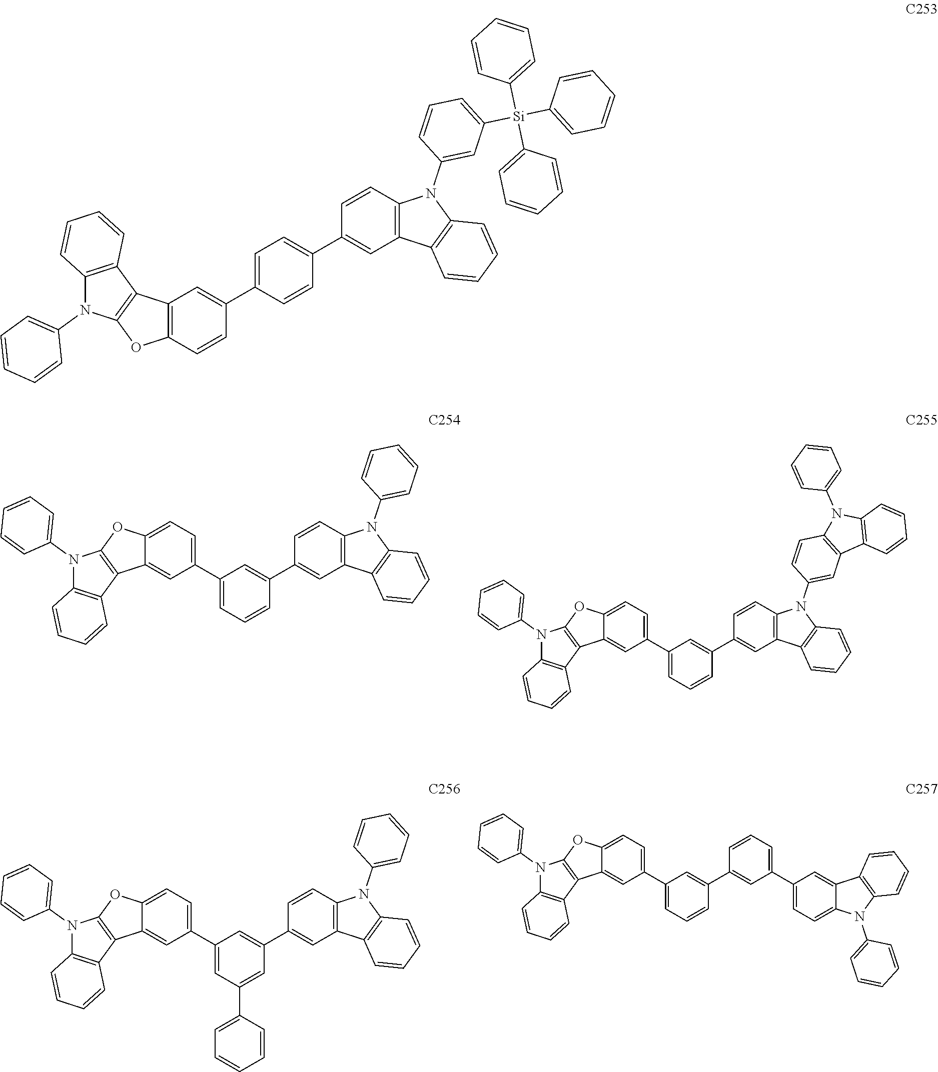 [SMILES: c1ccc(-n2c3ccccc3c3c4cc(-c5ccc(-c6ccc7c(c6)c6ccccc6n7-c6cccc([Si](c7ccccc7)(c7ccccc7)c7ccccc7)c6)cc5)ccc4oc32)cc1 ; c1ccc(-n2c3ccccc3c3cc(-c4cccc(-c5ccc6oc7c(c6c5)c5ccccc5n7-c5ccccc5)c4)ccc32)cc1 ; c1ccc(-n2c3ccccc3c3cc(-n4c5ccccc5c5cc(-c6cccc(-c7ccc8oc9c(c8c7)c7ccccc7n9-c7ccccc7)c6)ccc54)ccc32)cc1 ; c1ccc(-c2cc(-c3ccc4oc5c(c4c3)c3ccccc3n5-c3ccccc3)cc(-c3ccc4c(c3)c3ccccc3n4-c3ccccc3)c2)cc1 ; c1ccc(-n2c3ccccc3c3cc(-c4cccc(-c5cccc(-c6ccc7oc8c(c7c6)c6ccccc6n8-c6ccccc6)c5)c4)ccc32)cc1]

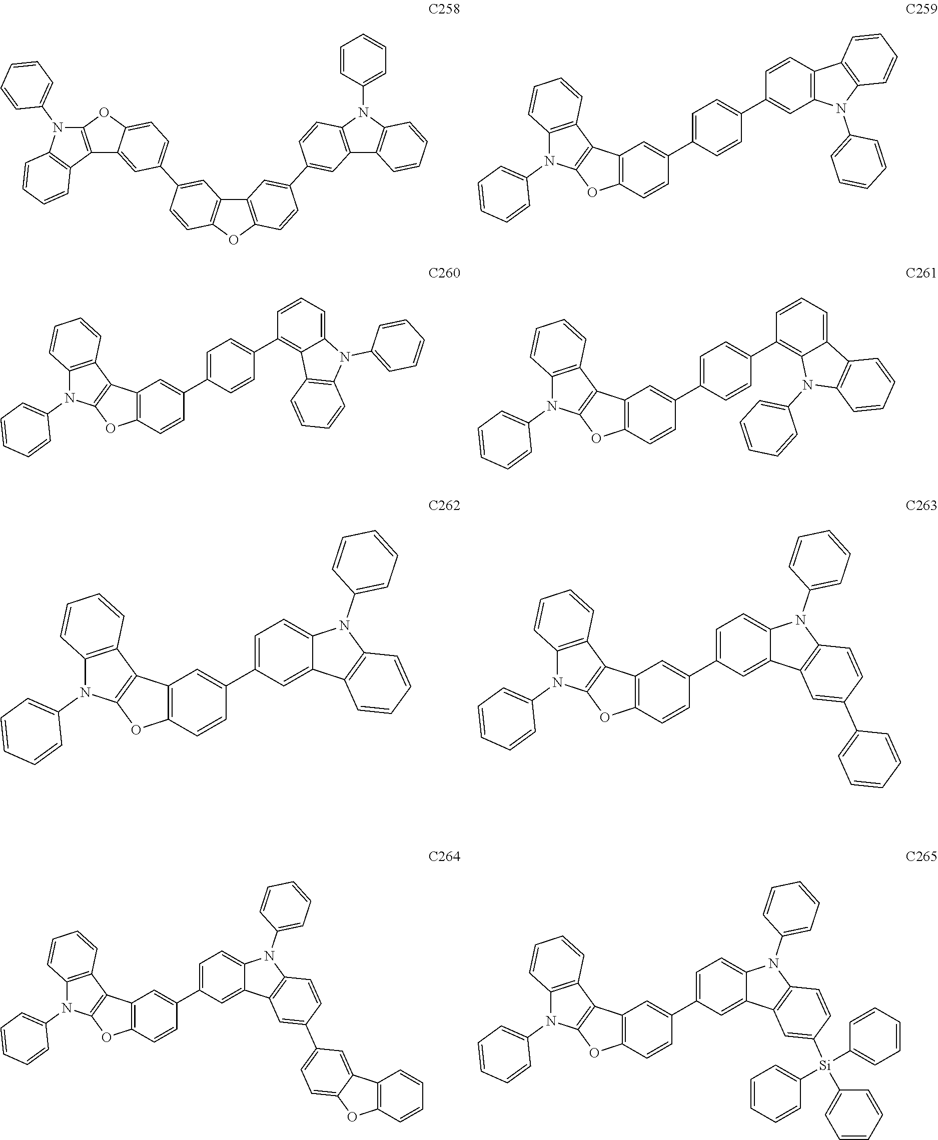

The hole transport region HTR may further include known materials in addition to the aforementioned nitrogen-containing compound. In an embodiment, the hole transport region HTR may include a compound represented by Formula H-1 below:

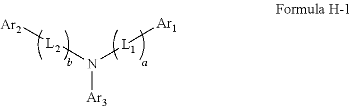

In Formula H-1 above, L₁ and L₂ may be each independently a direct linkage, a substituted or unsubstituted arylene group having 6 to 30 ring-forming carbon atoms, or a substituted or unsubstituted heteroarylene group having 2 to 30 ring-forming carbon atoms. a and b may be each independently an integer of 0 to 10. When a or b is an integer of 2 or greater, a plurality of L₁'s and L₂'s may be each independently a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may be each independently a substituted or unsubstituted aryl group having 6 to 30 ring-forming carbon atoms, or a substituted or unsubstituted heteroaryl group having 2 to 30 ring-forming carbon atoms. In addition, in Formula H-1, Ar₃ may be a substituted or unsubstituted aryl group having 6 to 30 ring-forming carbon atoms, or a substituted or unsubstituted heteroaryl group having 2 to 30 ring-forming carbon atoms.

The compound represented by Formula H-1 above may be a monoamine compound. Alternatively, the compound represented by Formula H-1 above may be a diamine compound in which at least one among Ar₁ to Ar₃ includes the amine group as a substituent. In addition, the compound represented by Formula H-1 above may be a carbazole-based compound including a substituted or unsubstituted carbazole group in at least one of Ar₁ or Ar₂, or a fluorene-based compound including a substituted or unsubstituted fluorene group in at least one of Ar₁ or Ar₂.

The compound represented by Formula H-1 may be represented by any one among the compounds of Compound Group H below. However, the compounds listed in Compound Group H below are examples, and the compounds represented by Formula H-1 are not limited to those represented by Compound Group H below:

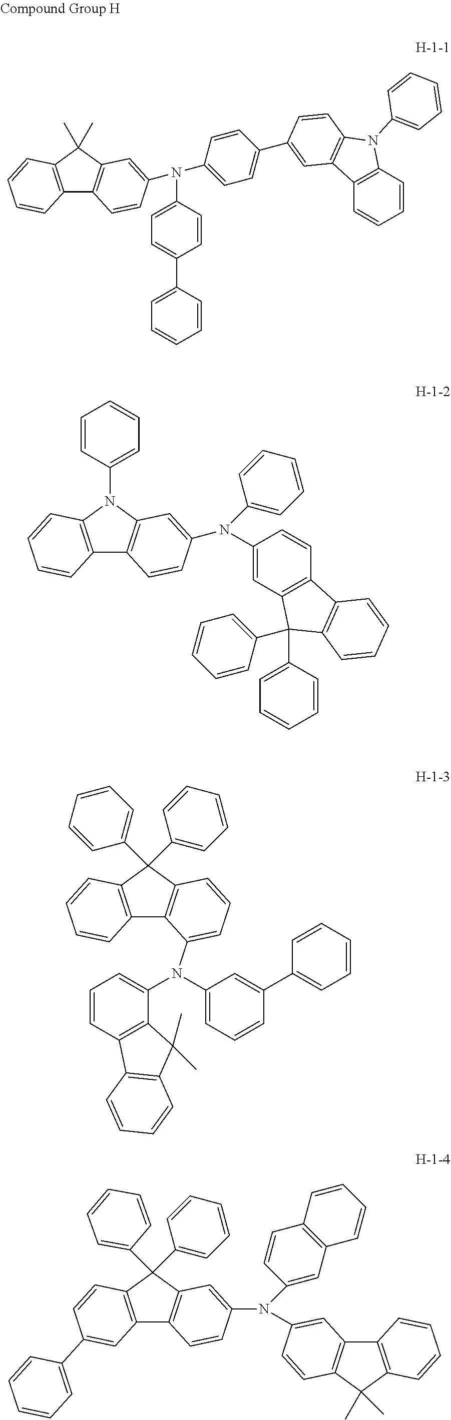 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

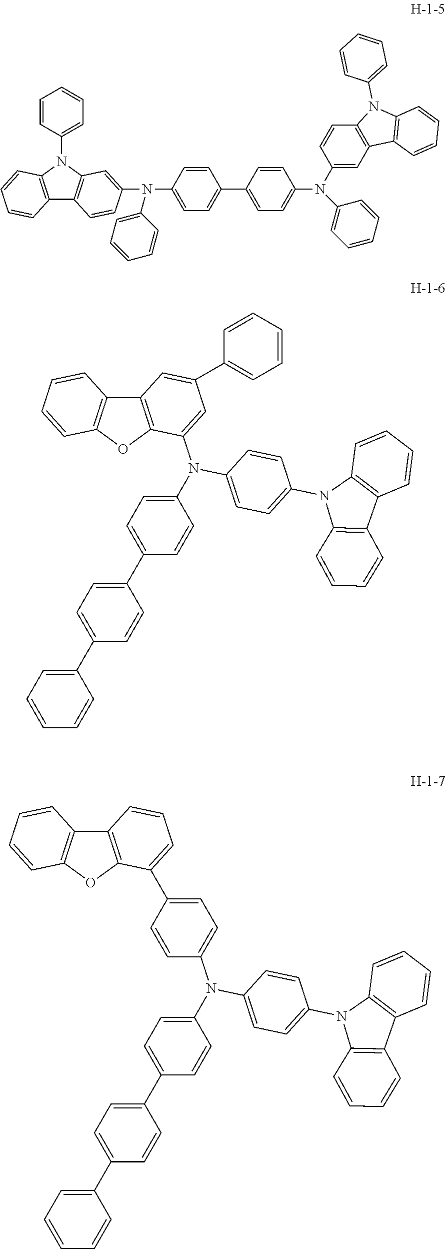 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

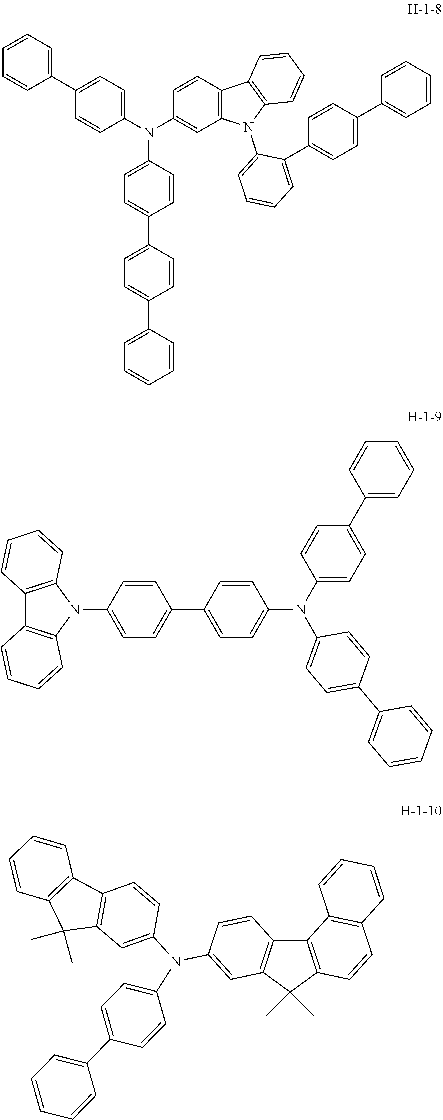 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

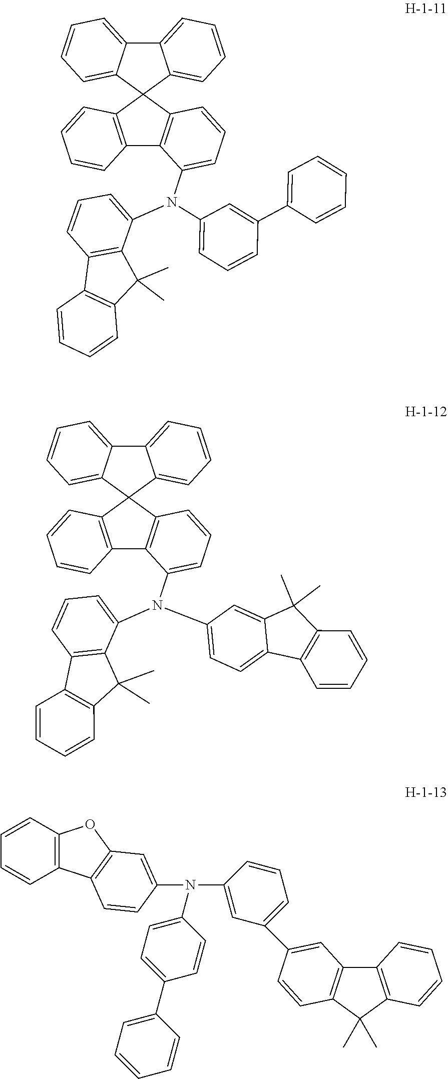 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

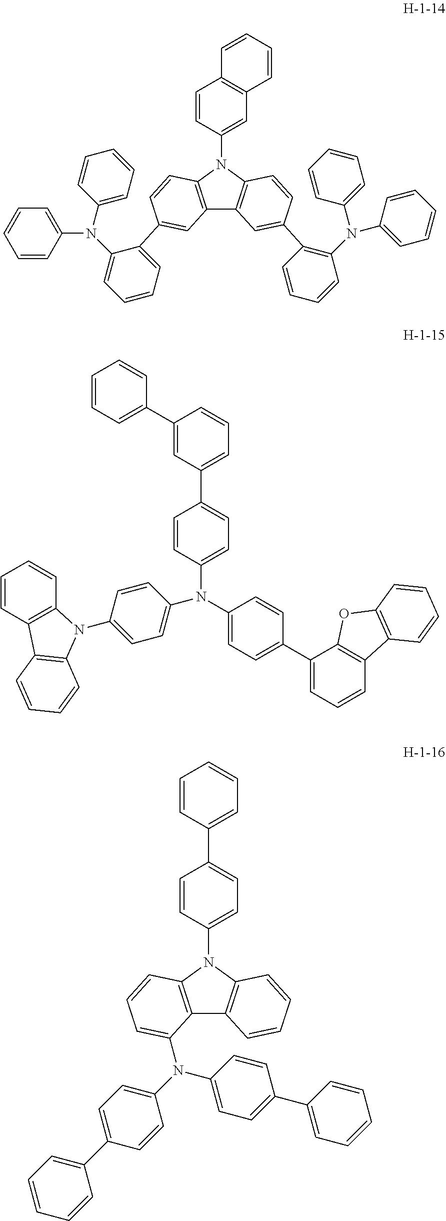 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

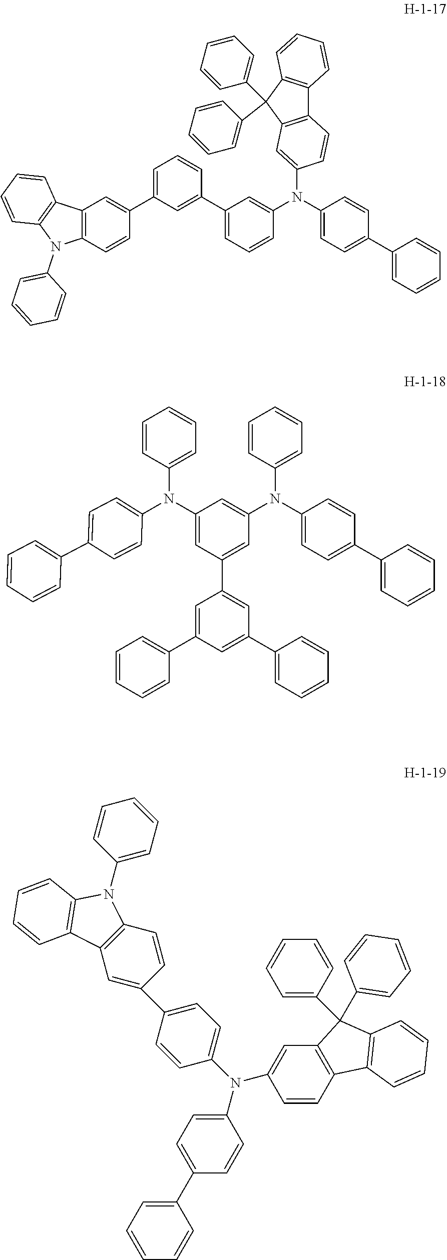 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound such as copper phthalocyanine; N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), etc.

The hole transport region HTR may include carbazole derivatives such as N-phenyl carbazole and polyvinyl carbazole, fluorene derivatives, N,N′-bis(3-methylphenyl)-N,N′-diphenyl-[1,1-biphenyl]-4,4′-diamine (TPD), triphenylamine derivatives such as 4,4′,4″-tris(N-carbazolyl)triphenylamine (TCTA), N,N′-di(naphthalene-1-yl)-N,N′-diphenyl-benzidine (NPB), 4,4′-cyclohexylidene bis[N,N-bis(4-methylphenyl]benzenamine] (TAPC), 4,4′-bis[N,N′-(3-tolyl)amino]-3,3′-dimethylbiphenyl (HMTPD), 1,3-di(carbazol-9-yl)benzene (mCP), etc.

In addition, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc. The hole transport region HTR may include the above-described compound of the hole transport region in at least one of a hole injection layer HIL, a hole transport layer HTL, or an electron blocking layer EBL.

The thickness of the hole transport region HTR may be from about 100 Å to about 10,000 Å, for example, from about 100 Å to about 5,000 Å. When the hole transport region HTR includes the hole injection layer HIL, the hole injection layer HIL may have, for example, a thickness of about 30 Å to about 1,000 Å. When the hole transport region HTR includes the hole transport layer HTL, the hole transport layer HTL may have a thickness of about 30 Å to about 1,000 Å. For example, when the hole transport region HTR includes the electron blocking layer EBL, the electron blocking layer EBL may have a thickness of about 10 Å to about 1,000 Å. If the thicknesses of the hole transport region HTR, the hole injection layer HIL, the hole transport layer HTL and the electron blocking layer EBL satisfy the above-described ranges, satisfactory hole transport properties may be achieved without a substantial increase in a driving voltage.

The hole transport region HTR may further include a charge generating material to increase conductivity in addition to the above-described materials. The charge generating material may be dispersed uniformly or non-uniformly in the hole transport region HTR. The charge generating material may be, for example, a p-dopant. The p-dopant may include at least one of a halogenated metal compound, a quinone derivative, a metal oxide, or a cyano group-containing compound, but the embodiments of the invention are not limited thereto. For example, the p-dopant may include metal halides such as CuI and RbI, quinone derivatives such as tetracyanoquinodimethane (TCNQ) and 2,3,5,6-tetrafluoro-7,7′,8,8′-tetracyanoquinodimethane (F4-TCNQ), metal oxides such as a tungsten oxide and a molybdenum oxide, dipyrazino[2,3-f: 2′,3′-h] quinoxaline-2,3,6,7,10,11-hexacarbonitrile (HATCN), 4-[[2,3-bis[cyano-(4-cyano-2,3,5,6-tetrafluorophenyl)methylidene]cyclopropylidene]-cyanomethyl]-2,3,5,6-tetrafluorobenzonitrile, etc., but the embodiments of the invention are not limited thereto.

As described above, the hole transport region HTR may further include at least one of the buffer layer or the electron blocking layer EBL in addition to the hole injection layer HIL and the hole transport layer HTL. The buffer layer may compensate for a resonance distance according to the wavelength of light emitted from the emission layer EML and, although not wanting to be bound by theory, may thus increase light emission efficiency. Materials which may be included in the hole transport region HTR may be used as materials to be included in the buffer layer. The electron blocking layer EBL is a layer that serves to prevent the electron injection from the electron transport region ETR to the hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer formed of a single material, a single layer formed of a plurality of different materials, or a multilayer structure having a plurality of layers formed of a plurality of different materials.

For example, the emission layer EML of the light emitting device ED may emit blue light in the region of about 490 nm or less. However, the embodiments of the invention are not limited thereto, and the emission layer EML may emit green light or red light.

In addition, in an embodiment, the emission layer EML includes a host and a dopant, and may include the above-described nitrogen-containing compound as a host. The nitrogen-containing compound represented by Formula 1 may be a host material of the emission layer.

For example, the emission layer EML in the light emitting device ED may include a host for emitting a phosphorescence and a dopant for emitting a phosphorescence, and may include the above-described nitrogen-containing compound as a host for emitting a phosphorescence. For example, the emission layer EML in the organic electroluminescence device ED may include a host for emitting a fluorescence and a dopant for emitting a fluorescence, and may include the above-described nitrogen-containing compound as a host for emitting a fluorescence.

The emission layer EML in the organic electroluminescence device ED may include a host for emitting a delayed fluorescence and a dopant for emitting a delayed fluorescence, and may include the above-described nitrogen-containing compound as a host for emitting a delayed fluorescence. The emission layer EML in the organic electroluminescence device ED may include a host for emitting a blue thermally activated delayed fluorescence (TADF) and a dopant for emitting a blue TADF, and may include the above-described nitrogen-containing compound as a host for emitting a blue TADF. The emission layer EML may include at least one of the nitrogen-containing compounds represented by Compound Group 1 as described above as a host material of the emission layer.

In the light emitting device ED, the emission layer EML may include a known material. The emission layer EML may include anthracene derivatives, pyrene derivatives, fluoranthene derivatives, chrysene derivatives, dehydrobenzanthracene derivatives, or triphenylene derivatives. The emission layer EML may include anthracene derivatives or pyrene derivatives.

In each light emitting device ED illustrated in FIGS. 3 to 6, the emission layer EML may include a host and a dopant, and the emission layer EML may include a compound represented by Formula E-1 below. The compound represented by Formula E-1 below may be used as a fluorescence host material or a delayed fluorescence host material.

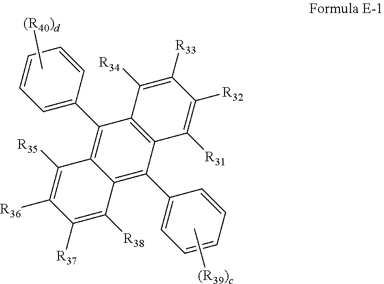

In Formula E-1, R₃₁ to R₄₀ may be each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted alkenyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. The groups R₃₁ to R₄₀ may be bonded to an adjacent group to form a saturated hydrocarbon ring or an unsaturated hydrocarbon ring. In addition, R₃₁ to R₄₀ may be bonded to an adjacent substituent or an adjacent benzene ring to form a fused ring.

In Formula E-1, c and d may be each independently an integer of 0 to 5.

Formula E-1 may be any one among the compounds represented by Compound Group E-1 below:

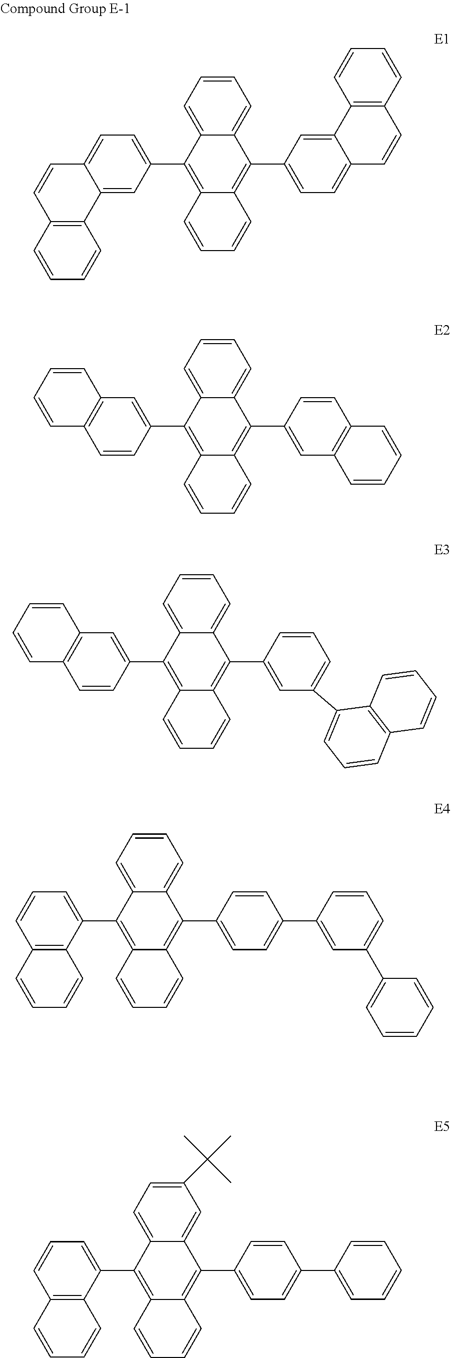

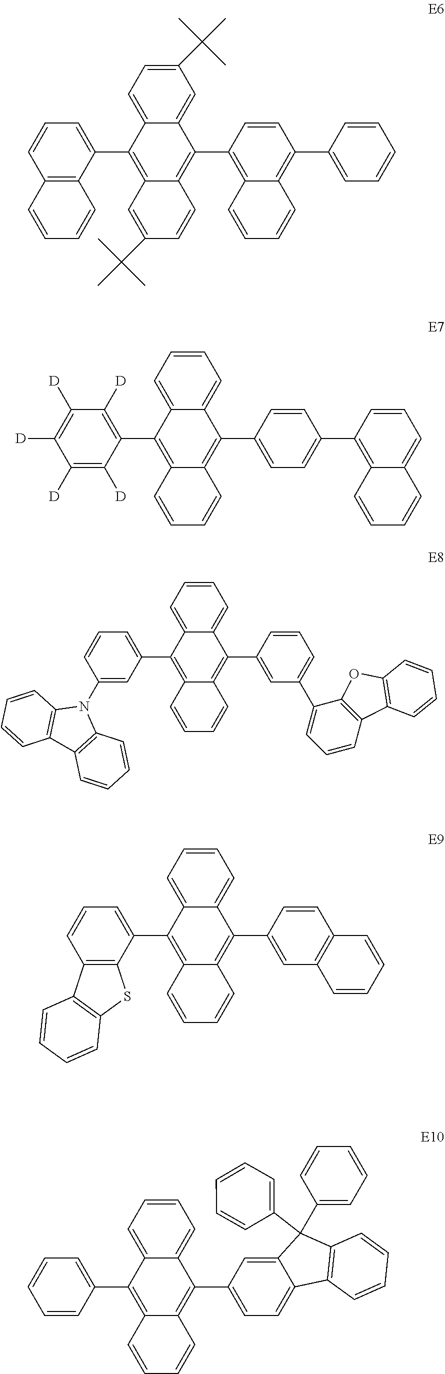

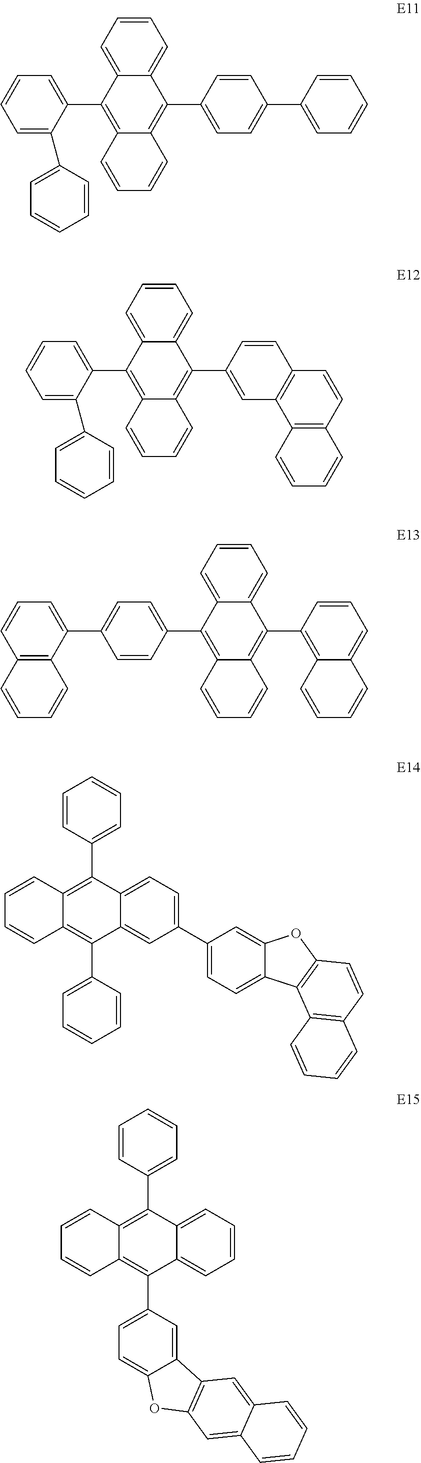

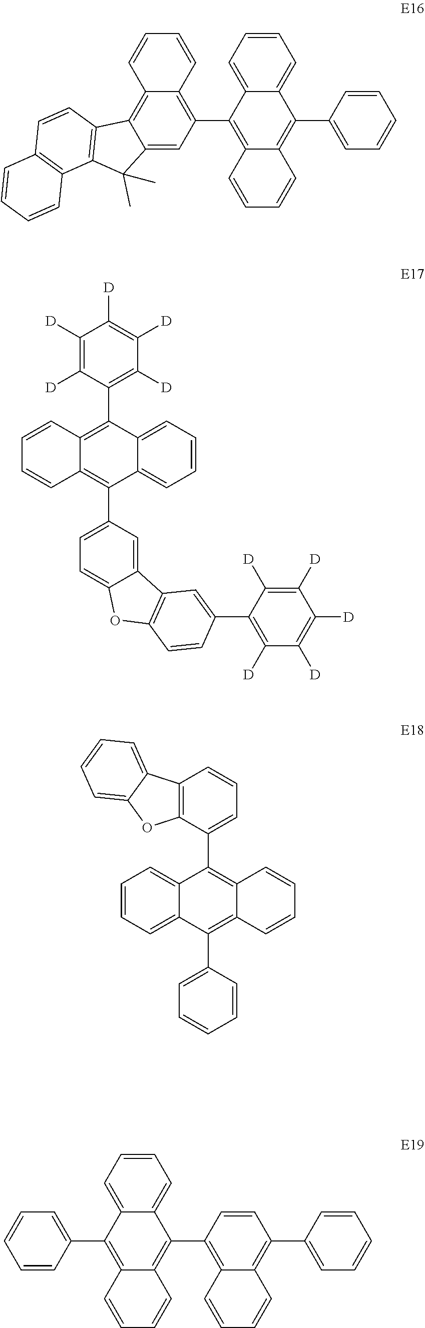

In an embodiment, the emission layer EML may include a compound represented by Formula E-2a or Formula E-2b below. The compound represented by Formula E-2a or Formula E-2b below may be used as a phosphorescence host material or a delayed fluorescence host material.

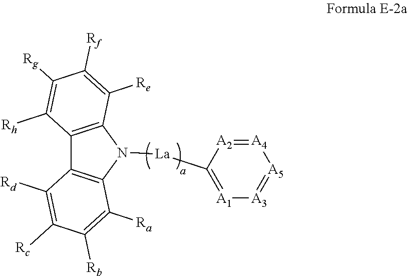

In Formula E-2a, and a may be an integer of 0 to 10, Lₐ may be a direct linkage, a substituted or unsubstituted arylene group having 6 to 30 ring-forming carbon atoms, or a substituted or unsubstituted heteroarylene group having 2 to 30 ring-forming carbon atoms. When a is an integer of 2 or more, a plurality of Lₐ's may be each independently a substituted or unsubstituted arylene group having 6 to 30 ring-forming carbon atoms, or a substituted or unsubstituted heteroarylene group having 2 to 30 ring-forming carbon atoms.

In addition, in Formula E-2a, A₁ to A₅ may be each independently N or CRᵢ. Rₐ to Rᵢ may be each independently a hydrogen atom, a deuterium atom, a substituted or unsubstituted amine group, a substituted or unsubstituted thio group, a substituted or unsubstituted oxy group, a substituted or unsubstituted oxide group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. The groups Rₐ to Rᵢ may be bonded to an adjacent group to form a hydrocarbon ring or a heterocycle containing N, O, S, etc. as a ring-forming atom. In Formula E-2a, two or three selected from among A₁ to A₅ may be N, and the rest may be CRᵢ.

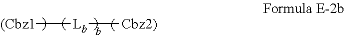

In Formula E-2b, Cbz1 and Cbz2 may be each independently an unsubstituted carbazole group, or a carbazole group substituted with an aryl group having 6 to 30 ring-forming carbon atoms. The variable L_{b} is a direct linkage, a substituted or unsubstituted arylene group having 6 to 30 ring-forming carbon atoms, or a substituted or unsubstituted heteroarylene group having 2 to 30 ring-forming carbon atoms. The variable b is an integer of 0 to 10, and when b is an integer of 2 or more, a plurality of L_{b}'s may be each independently a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one among the compounds of Compound Group E-2 below. However, the compounds listed in Compound Group E-2 below are examples, the compound represented by Formula E-2a or Formula E-2b is not limited to those represented by Compound Group E-2 below.

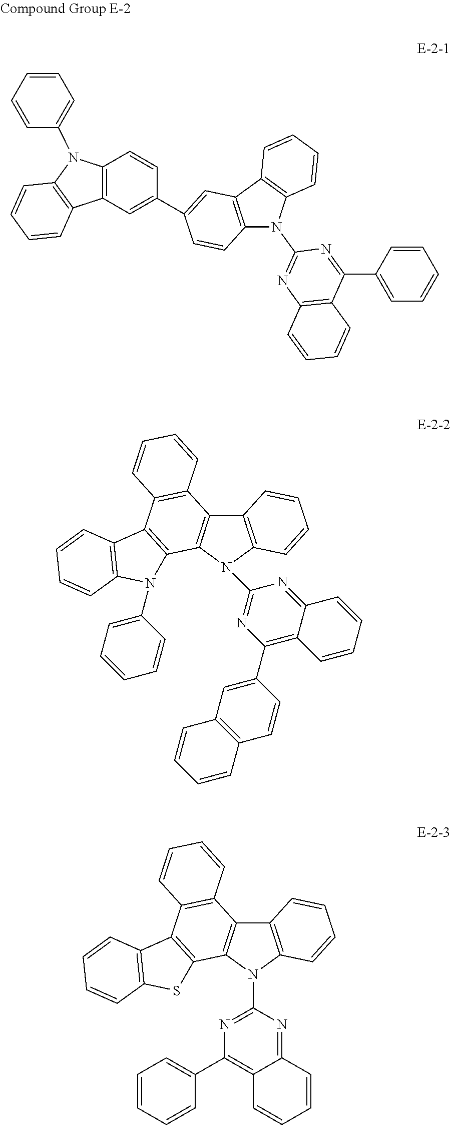 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

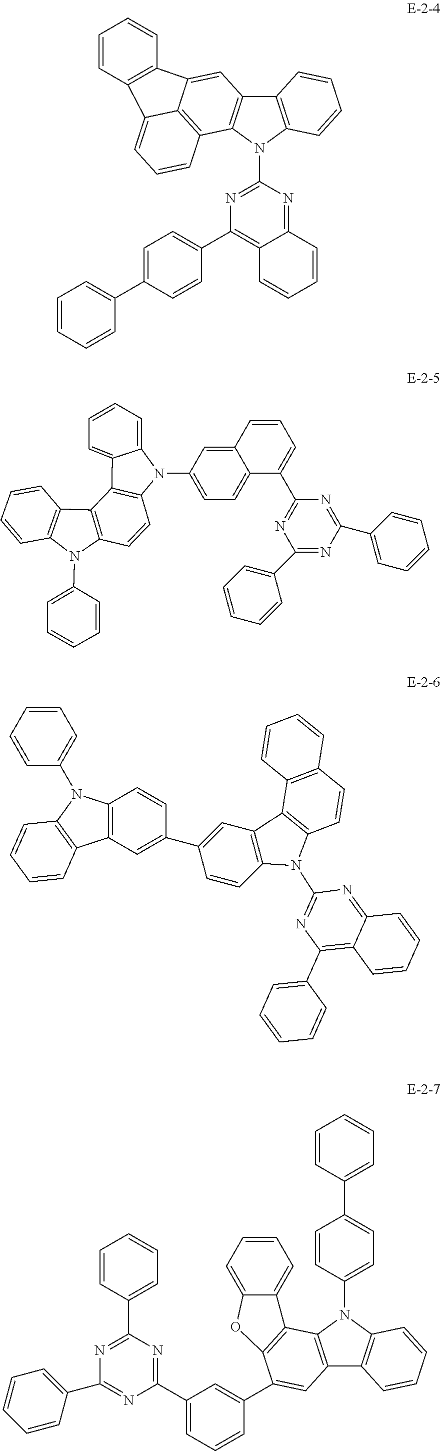 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1]

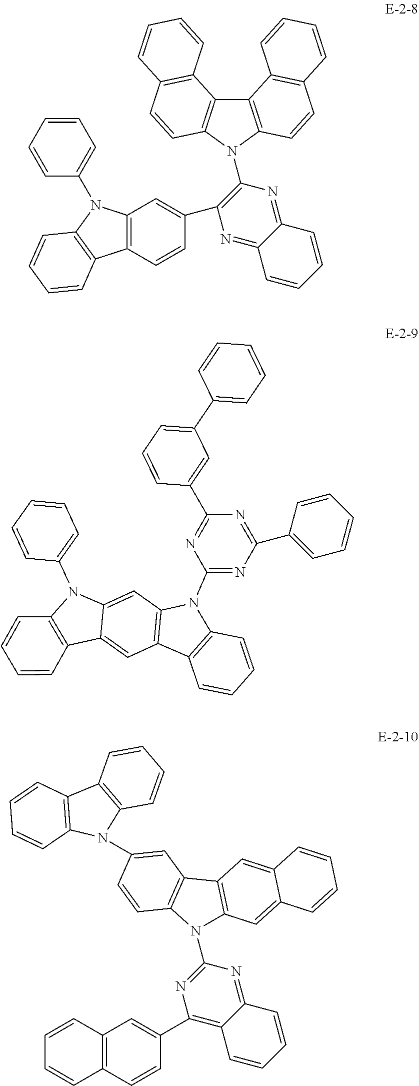 [SMILES: c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1]

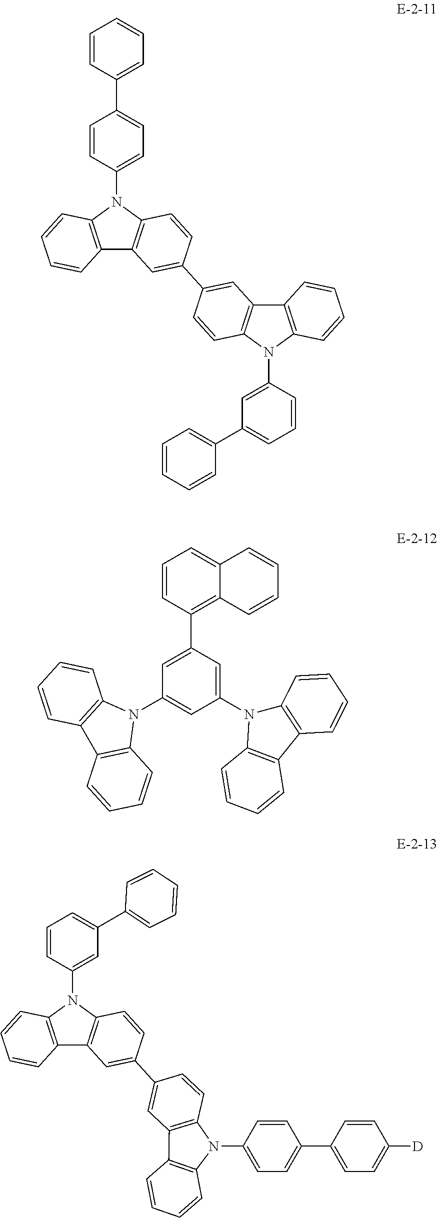 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1]

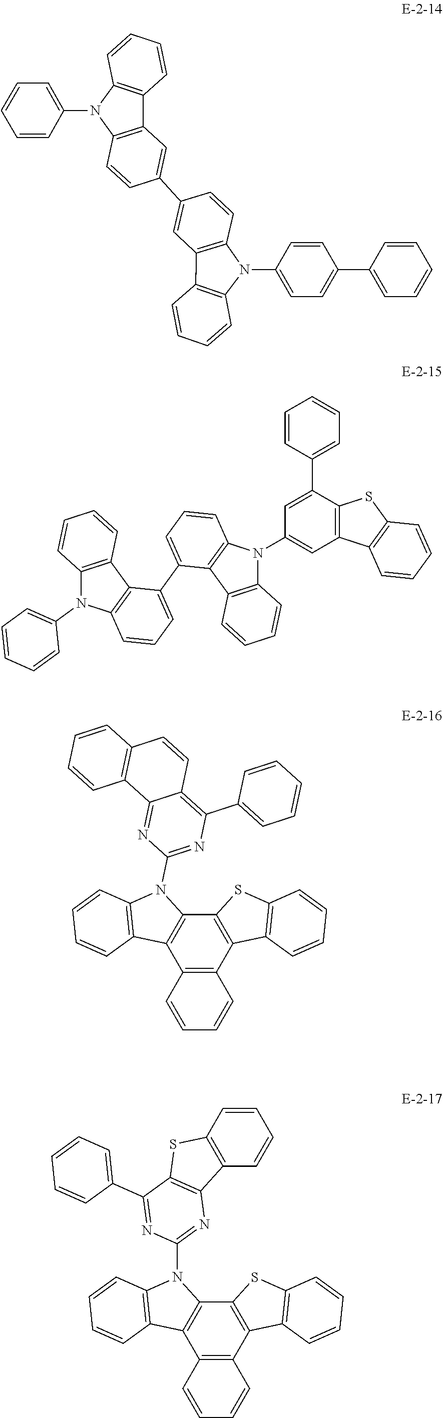 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1]

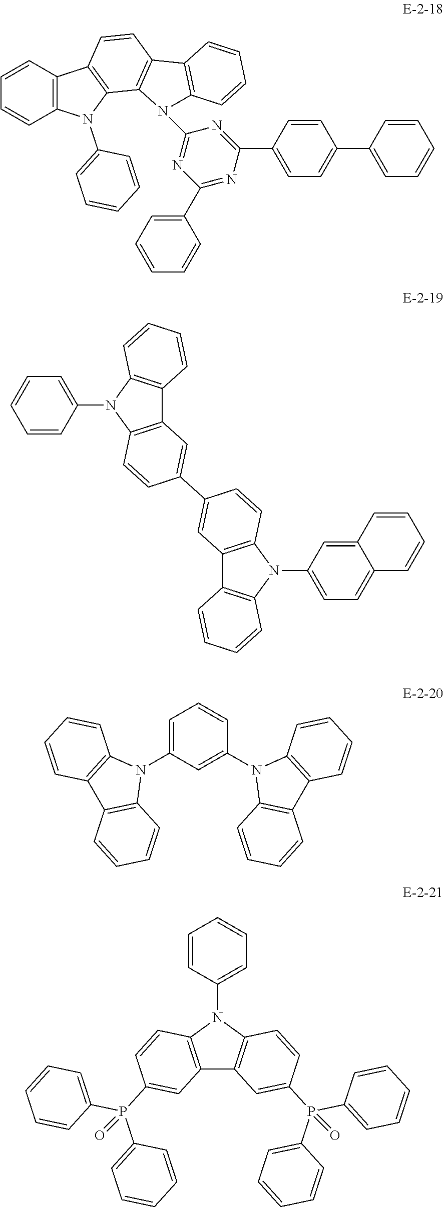 [SMILES: c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; O=P(c1ccccc1)(c1ccccc1)c1ccc2c(c1)c1cc(P(=O)(c3ccccc3)c3ccccc3)ccc1n2-c1ccccc1]

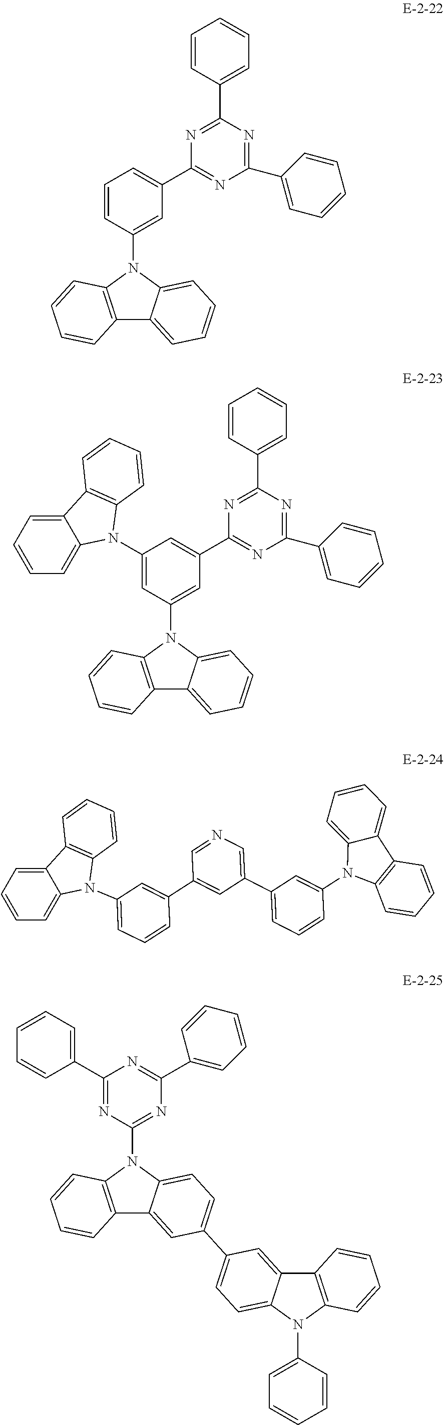 [SMILES: c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a general material known in the art as a host material. For example, the emission layer EML may include, as a host material, at least one of bis[2-(diphenylphosphino)phenyl] ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, the embodiments of the invention are not limited thereto, and for example, tris(8-hydroxyquinolino)aluminum (Alq₃), 4,4′-bis(N-carbazolyl)-1,1′-biphenyl (CBP), poly(N-vinylcarbazole) (PVK), 9,10-di(naphthalene-2-yl)anthracene (ADN), 4,4′,4″-tris(carbazol-9-yl)-triphenylamine (TCTA), 1,3,5-tris(N-phenylbenzimidazole-2-yl)benzene (TPBi), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenylcyclotriphosphazene (CP1), 1,4-bis(triphenylsilyl)benzene (UGH2), hexaphenylcyclotrisiloxane (DPSiO₃), octaphenylcyclotetra siloxane (DPSiO₄), 2,8-bis(diphenylphosphoryl)dibenzofuran (PPF), etc. may be used as a host material.

The emission layer EML may include a compound represented by Formula M-a or Formula M-b below. The compound represented by Formula M-a or Formula M-b below may be used as a phosphorescence dopant material.

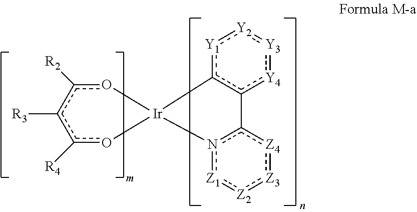

In Formula M-a above, Y₁ to Y₄ and Z₁ to Z₄ may be each independently CR₁ or N, R₁ to R₄ may be each independently a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In Formula M-a, m is 0 or 1, and n is 2 or 3. In Formula M-a, when m is 0, n is 3, and when m is 1, n is 2. The compound represented by Formula M-a may be used as a red phosphorescence dopant or a green phosphorescence dopant.

The compound represented by Formula M-a may be represented by any one among Compound M-a1 to Compound M-a19 below. However, Compounds M-a1 to M-a19 below are examples, and the compound represented by Formula M-a is not limited to those represented by Compounds M-a1 to M-a19 below:

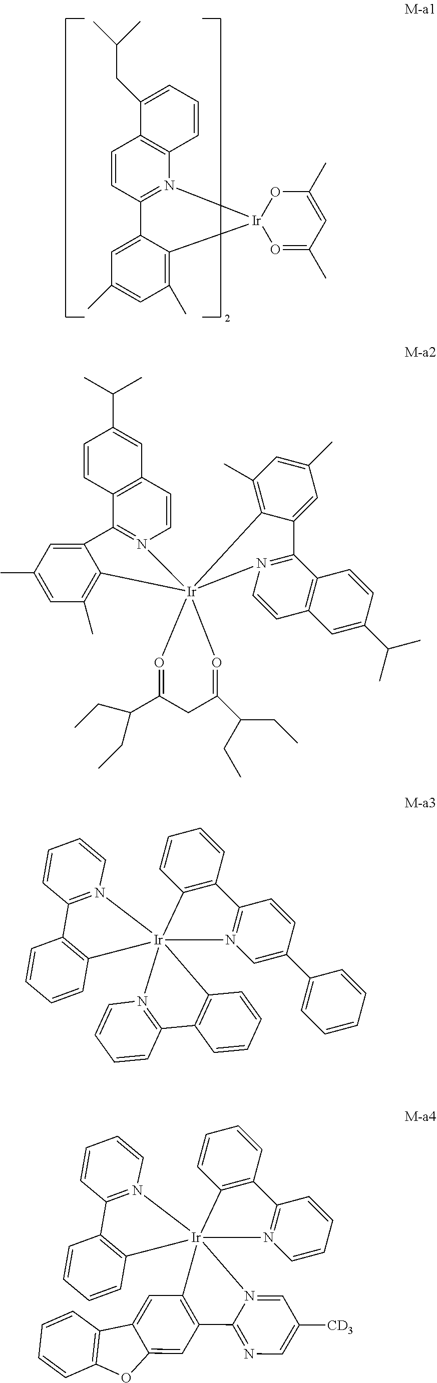

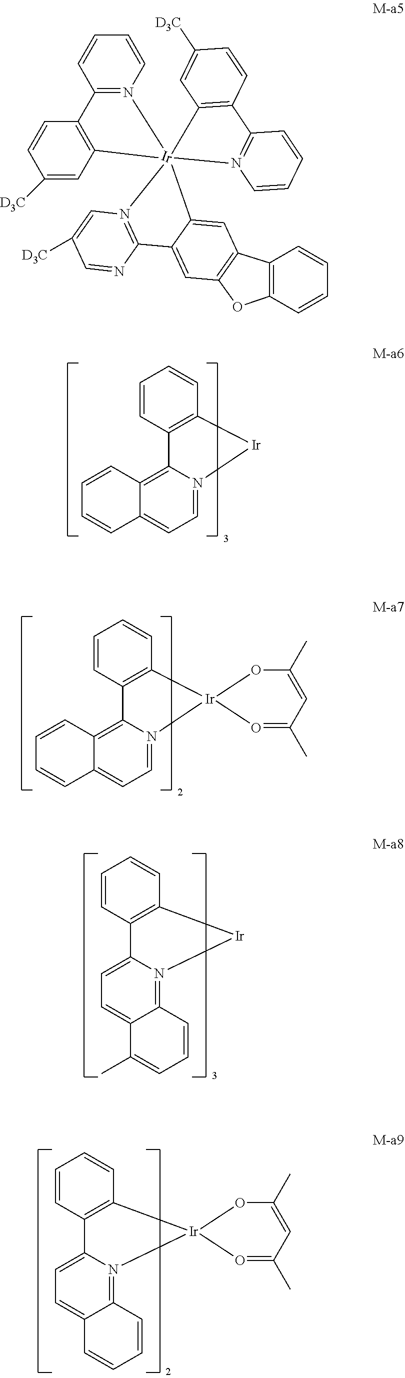

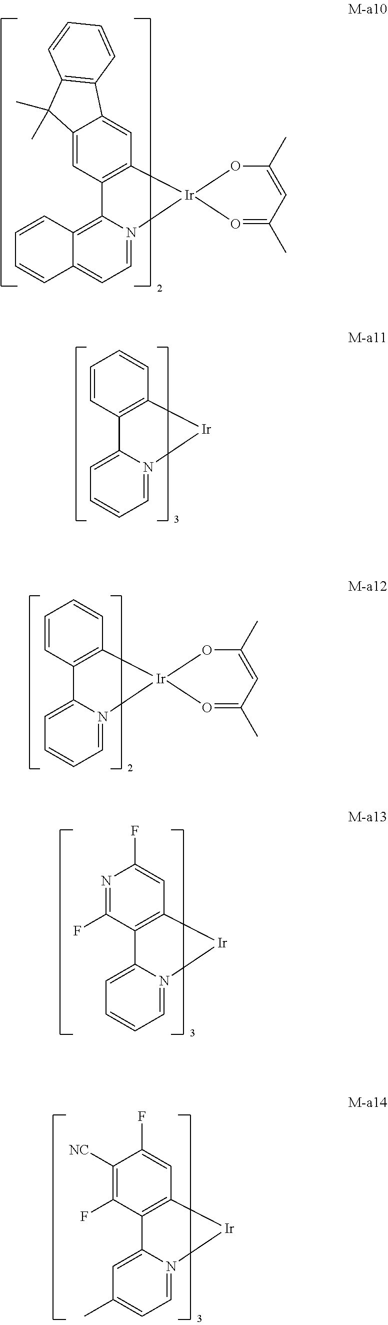

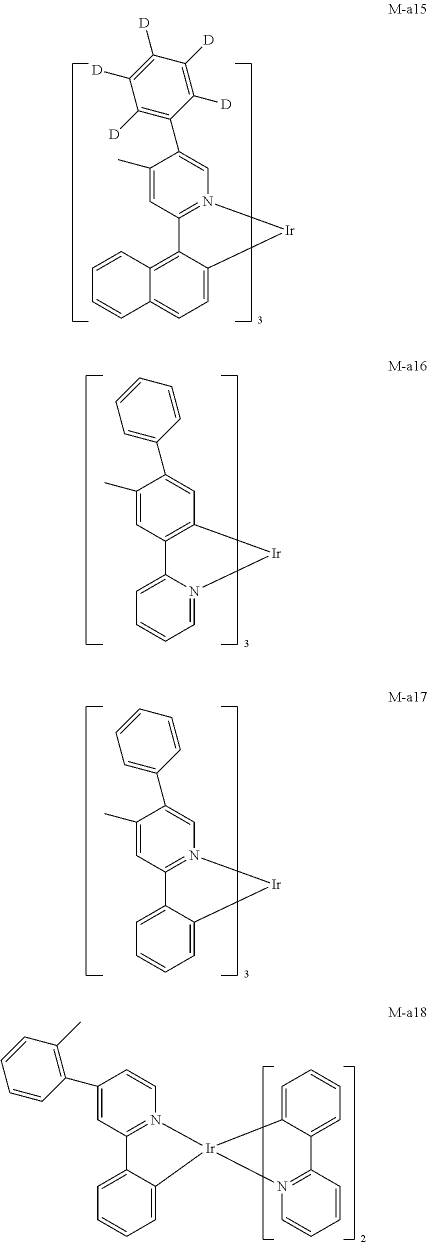

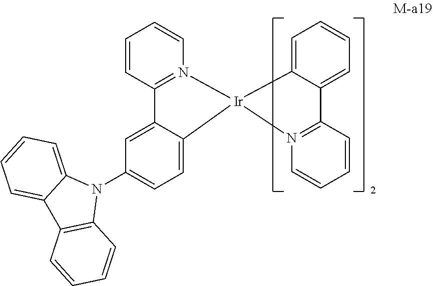

Compound M-a1 and Compound M-a2 may be used as a red dopant material, and Compound M-a3 to Compound M-a5 may be used as a green dopant material.

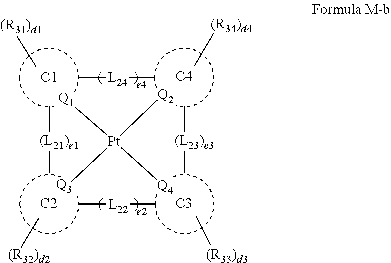

In Formula M-b, Q₁ to Q₄ are each independently C or N, and C1 to C4 are each independently a substituted or unsubstituted hydrocarbon ring having 5 to 30 ring-forming carbon atoms, or a substituted or unsubstituted heterocycle having 2 to 30 ring-forming carbon atoms. L₂₁ to L₂₄ are each independently a direct linkage,

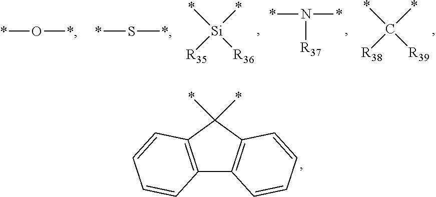

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, and e1 to e4 are each independently 0 or 1. R₃₁ to R₃₉ are each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring, and d1 to d4 are each independently an integer of 0 to 4. The compound represented by Formula M-b may be used as a blue phosphorescence dopant or a green phosphorescence dopant.

The compound represented by Formula M-b may be represented by any one among the compounds below. However, the compounds below are examples, and the compound represented by Formula M-b is not limited to those represented by the compounds below:

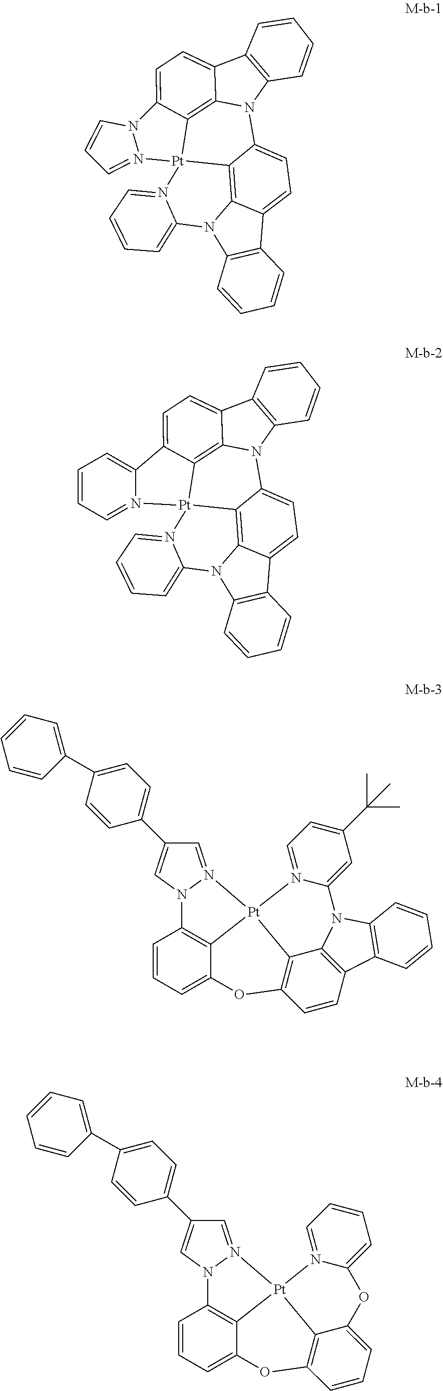

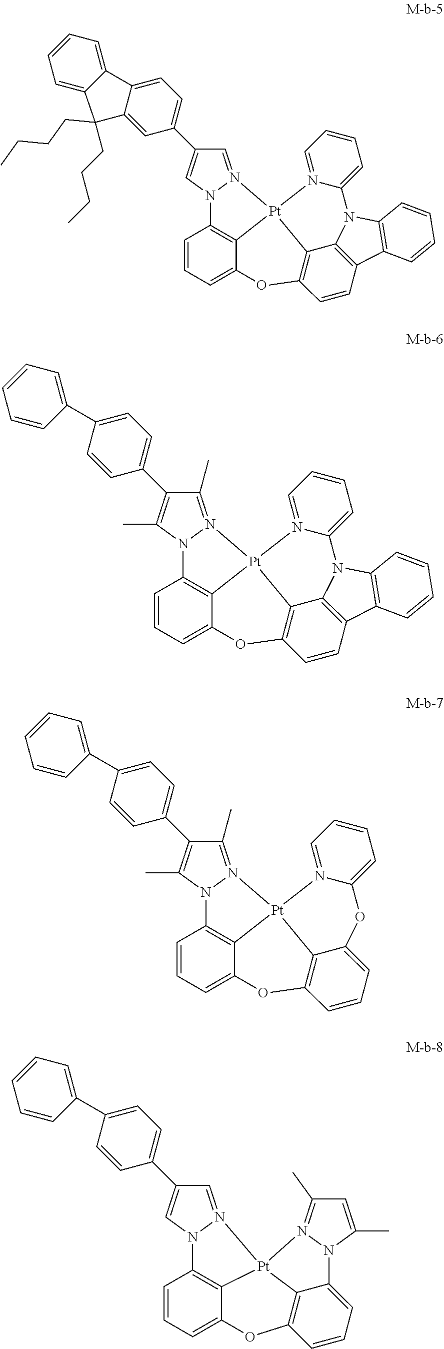

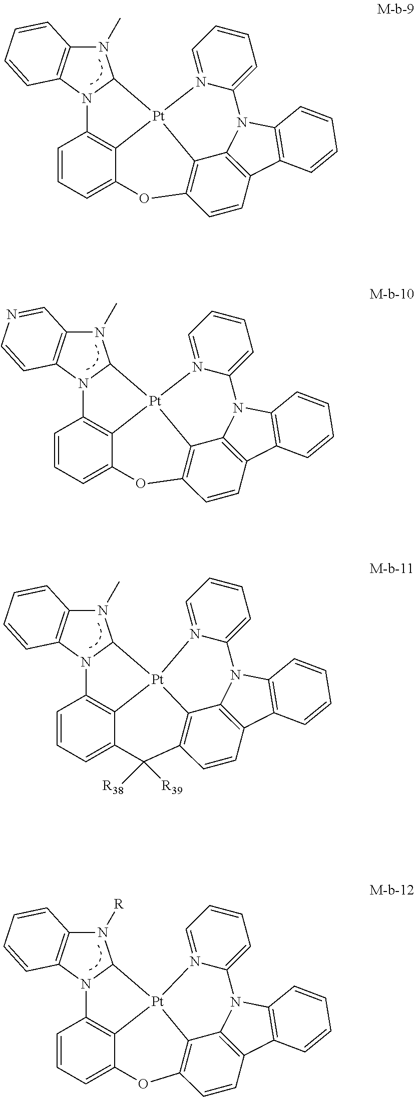

In the compounds, R, R₃₈, and R₃₉ may be each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

The emission layer EML may include a compound represented by any one among Formula F-a to Formula F-c below. The compound represented by Formula F-a or Formula F-c below may be used as a fluorescence dopant material.

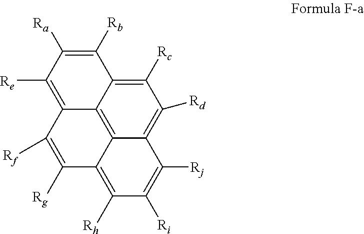

In Formula F-a, two selected from among Rₐ to Rⱼ may each independently be substituted with

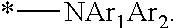

The others, which are not substituted with

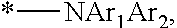

among Rₐ to Rⱼ may be each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In

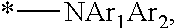

Ar₁ and Ar₂ may be each independently a substituted or unsubstituted aryl group having 6 to 30 ring-forming carbon atoms, or a substituted or unsubstituted heteroaryl group having 2 to 30 ring-forming carbon atoms. For example, at least one of Ar₁ or Ar₂ may be a heteroaryl group containing O or S as a ring-forming atom.

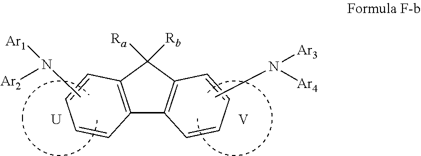

In Formula F-b, Rₐ and R_{b} may be each independently a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring.

In Formula F-b, An to Ar₄ may be each independently a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring.

In Formula F-b, U and V may be each independently a substituted or unsubstituted hydrocarbon ring having 5 to 30 ring-forming carbon atoms, or a substituted or unsubstituted heterocycle having 2 to 30 ring-forming carbon atoms.

In Formula F-b, the number of rings represented by U and V may be each independently 0 or 1. For example, in Formula F-b, it means that when the number of U or V is 1, one ring forms a condensed ring at a part described as U or V, and when the number of U or V is 0, a ring described as U or V is not present. Specifically, when the number of U is 0 and the number of V is 1, or when the number of U is 1 and the number of V is 0, the condensed ring having a fluorene core of Formula F-b may be a four-ring cyclic compound. In addition, when each number of U and V is 0, the condensed ring of Formula F-b may be a three-ring cyclic compound. In addition, when each number of U and V is 1, the condensed ring having a fluorene core of Formula F-b may be a five-ring cyclic compound.

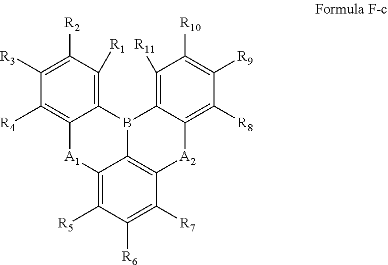

In Formula F-c, A₁ and A₂ may be each independently O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. The groups R₁ to R₁₁ are each independently a hydrogen atom, a deuterium atom, a halogen atom, a cyano group, a substituted or unsubstituted amine group, a substituted or unsubstituted boron group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring.

In Formula F-c, A₁ and A₂ may each independently be bonded to substituents of an adjacent ring to form a condensed ring. For example, when A₁ and A₂ are each independently NRₘ, A₁ may be bonded to R₄ or R₅ to form a ring. In addition, A₂ may be bonded to R₇ or R₈ to form a ring.

In an embodiment, the emission layer EML may include, as a known dopant material, styryl derivatives (e.g., 1,4-bis[2-(3-N-ethylcarbazoryl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl)-N-phenylbenzenamine (N-BDAVBi), 4,4′-bis[2-(4-(N,N-diphenylamino)phenyl)vinyl]biphenyl(DPAVBi), perylene and the derivatives thereof (e.g., 2,5,8,11-tetra-t-butylperylene (TBP)), pyrene and the derivatives thereof (e.g., 1,1-dipyrene, 1,4-dipyrenylbenzene, 1,4-bis(N,N-diphenylamino)pyrene), etc.

The emission layer EML may include a known phosphorescence dopant material. For example, a metal complex including iridium (Ir), platinum (Pt), osmium (Os), aurum (Au), titanium (Ti), zirconium (Zr), hafnium (Hf), europium (Eu), terbium (Tb), or thulium (Tm) may be used as a phosphorescence dopant. Specifically, iridium(III) bis(4,6-difluorophenylpyridinato-N,C2′)picolinate (FIrpic), bis(2,4-difluorophenylpyridinato)-tetrakis(1-pyrazolyl)borate iridium(III) (Fir6), or platinum octaethyl porphyrin (PtOEP) may be used as a phosphorescence dopant. However, the embodiments of the invention are not limited thereto.

The emission layer EML may include a quantum dot material. The core of the quantum dot may be selected from among a compound of Groups II-VI, a compound of Groups III-VI, a compound of Groups I, III, and IV, a compound of Groups III-V, a compound of Groups IV-VI, an element of Group IV, a compound of Group IV, and a combination thereof.

The compound of Groups II-VI may be selected from the group consisting of a binary compound selected from the group consisting of CdSe, CdTe, CdS, ZnS, ZnSe, ZnTe, ZnO, HgS, HgSe, HgTe, MgSe, MgS, and a mixture thereof, a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof, and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and a mixture thereof.

The compound of Groups III-VI may include a binary compound such as In₂S₃ and In₂Se₃, a ternary compound such as InGaS₃ and InGaSe₃, or any combination thereof. The compound of Groups I, III, and VI may be selected from a ternary compound selected from the group consisting of AgInS, AgInS₂, CuInS, CuInS₂, AgGaS₂, CuGaS₂ CuGaO₂, AgGaO₂, AgAlO₂, and a mixture thereof, or a quaternary compound such as AgInGaS₂ and CuInGaS₂.

The compound of Groups III-V may be selected from the group consisting of a binary compound selected from the group consisting of GaN, GaP, GaAs, GaSb, AlN, AlP, AlAs, AlSb, InN, InP, InAs, InSb, and a mixture thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and a mixture thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and a mixture thereof. The compound of Groups III-V may further include a Group II metal. For example, InZnP, etc. may be selected as a compound of Groups III, II, and V compound.

The compound of Groups IV-VI may be selected from the group consisting of a binary compound selected from the group consisting of SnS, SnSe, SnTe, PbS, PbSe, PbTe, and a mixture thereof, a ternary compound selected from the group consisting of SnSeS, SnSeTe, SnSTe, PbSeS, PbSeTe, PbSTe, SnPbS, SnPbSe, SnPbTe, and a mixture thereof, and a quaternary compound selected from the group consisting of SnPbSSe, SnPbSeTe, SnPbSTe, and a mixture thereof. The element of Group IV may be selected from the group consisting of Si, Ge, and a mixture thereof. The compound of Group IV may be a binary compound selected from the group consisting of SiC, SiGe, and a mixture thereof.

In this case, a binary compound, a ternary compound, or a quaternary compound may be present in particles in a uniform concentration distribution, or may be present in the same particle in a partially different concentration distribution. In addition, the quantum dot may have a core/shell structure in which one quantum dot surrounds another quantum dot. In a core/shell structure, the interface of the shell may have a concentration gradient in which the concentration of an element present in the shell becomes lower towards the core.

In some embodiments, a quantum dot may have the above-described core-shell structure including a core containing nanocrystals and a shell surrounding the core. The shell of the quantum dot may serve as a protection layer to prevent the chemical deformation of the core so as to maintain semiconductor properties, and/or a charging layer to impart electrophoresis properties to the quantum dot. The shell may be a single layer or a multilayer. An example of the shell of the quantum dot may include a metal or a non-metal oxide, a semiconductor compound, or a combination thereof.

For example, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO, or a ternary compound such as MgAl₂O₄, CoFe₂O₄, NiFe₂O₄, or CoMn₂O₄, but the embodiments are not limited thereto.

Also, the semiconductor compound may be, for example, CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but the embodiments are not limited thereto.

The quantum dot may have a full width of half maximum (FWHM) of a light emission wavelength spectrum of about 45 nm or less, preferably about 40 nm or less, and more preferably about 30 nm or less, and color purity or color reproducibility may be improved in the above range. In addition, light emitted through such a quantum dot is emitted in all directions, and thus a wide viewing angle may be improved.

In addition, although the form of a quantum dot is not particularly limited as long as it is a form commonly used in the art, more specifically, a quantum dot in the form of a generally spherical nanoparticle, a generally pyramidal nanoparticle, a generally multi-armed nanoparticle, a generally cubic nanoparticle, a generally nanotube-shaped nanoparticle, a generally nanowire-shaped nanoparticle, a generally nanofiber-shaped nanoparticle, and other nanoparticles, etc. may be used.

The quantum dot may control the color of emitted light according to the particle size thereof, and accordingly, the quantum dot may have various emission colors such as blue, red, and green. In each light emitting device ED illustrated in FIGS. 3 to 6, the electron transport region ETR is provided on the emission layer EML. The electron transport region ETR may include at least one of the hole blocking layer HBL, the electron transport layer ETL, or the electron injection layer EIL, but the embodiments of the invention are not limited thereto.

The electron transport region ETR may have a single layer formed of a single material, a single layer formed of a plurality of different materials, or a multilayer structure including a plurality of layers formed of a plurality of different materials.

For example, the electron transport region ETR may have a single layer structure of the electron injection layer EIL or the electron transport layer ETL, and may have a single layer structure formed of an electron injection material and an electron transport material. In addition, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL are stacked in order from the emission layer EML, but the embodiments of the invention are not limited thereto. The electron transport region ETR may have a thickness, for example, from about 1,000 Å to about 1,500 Å.

The electron transport region ETR may be formed by using various methods such as a vacuum deposition method, a spin coating method, a cast method, a Langmuir-Blodgett (LB) method, an inkjet printing method, a laser printing method, a laser induced thermal imaging (LITI) method, etc.

The electron transport region ETR may include a compound represented by Formula ET-1 below:

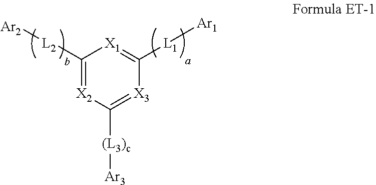

In Formula ET-1, at least one among X₁ to X₃ is N, and the rest are CRₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may be each independently a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, a to c may be each independently an integer of 0 to 10. In Formula ET-1, L₁ to L₃ may be each independently a direct linkage, a substituted or unsubstituted arylene group having 6 to 30 ring-forming carbon atoms, or a substituted or unsubstituted heteroarylene group having 2 to 30 ring-forming carbon atoms. When a to c are an integer of 2 or more, L₁ to L₃ may be each independently a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. The embodiments of the invention are not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebg₂), 9,10-di(naphthalene-2-yl)anthracene (ADN), 1,3-Bis[3,5-di(pyridin-3-yl)phenyl]benzene (BmPyPhB), or a mixture thereof.

The electron transport region ETR may include at least one among Compound ET1 to Compound ET35 below:

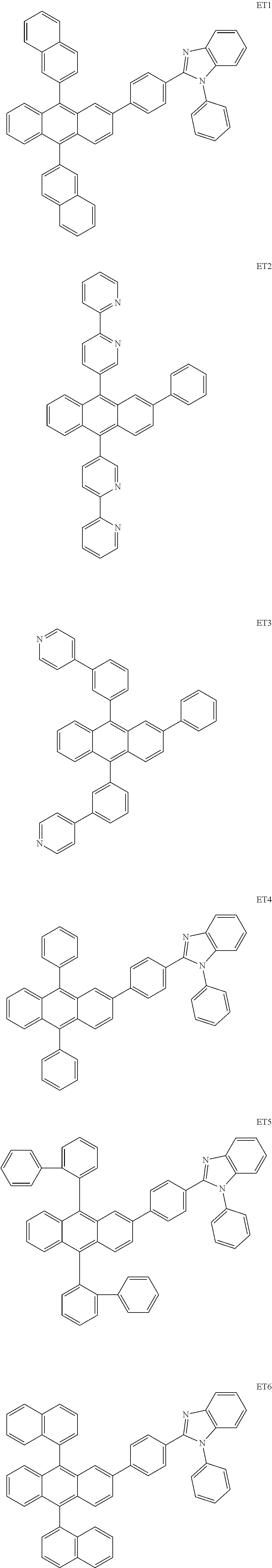

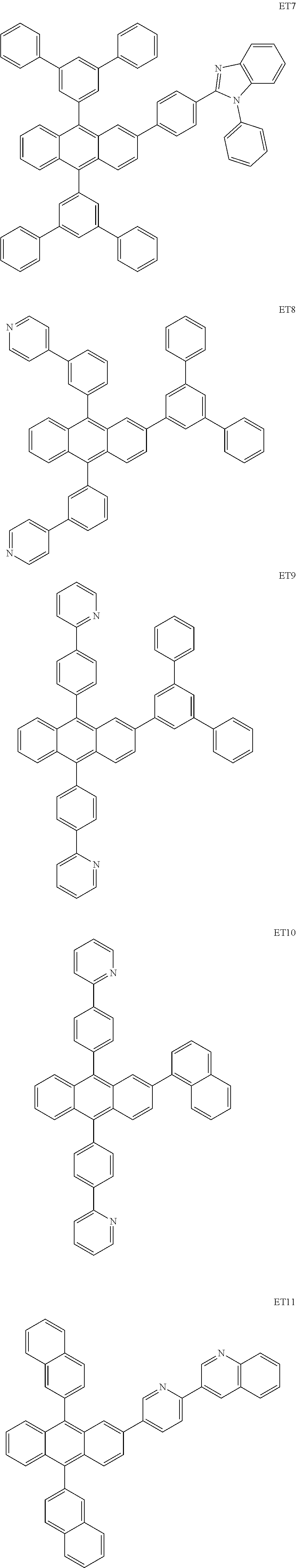 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccncc6)c5)c5ccccc5c(-c5cccc(-c6ccncc6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1]

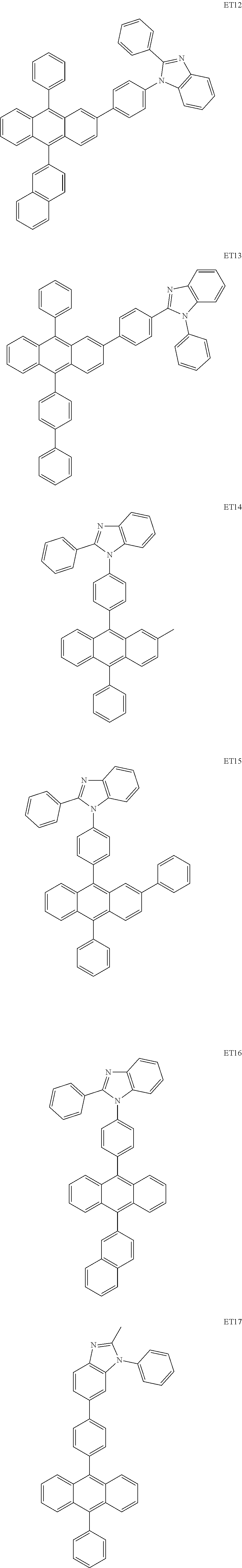 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5ccc(-c6nc7ccccc7n6-c6ccccc6)cc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

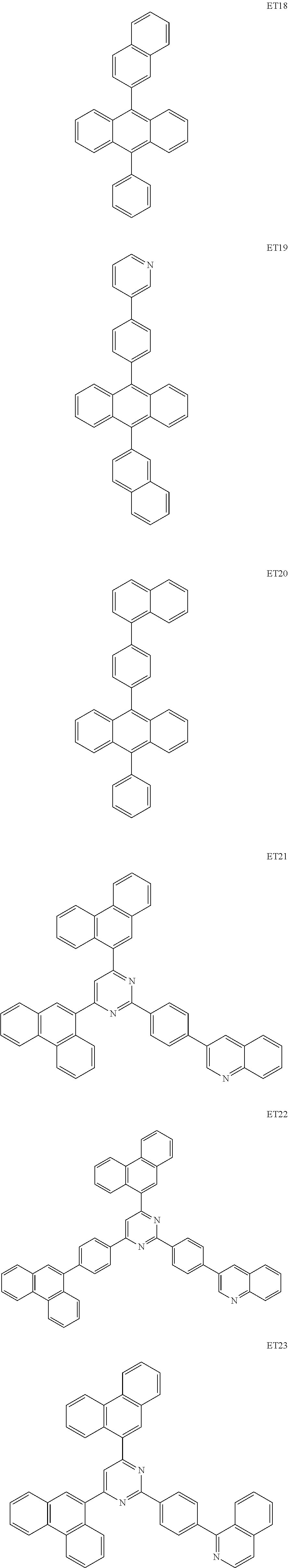

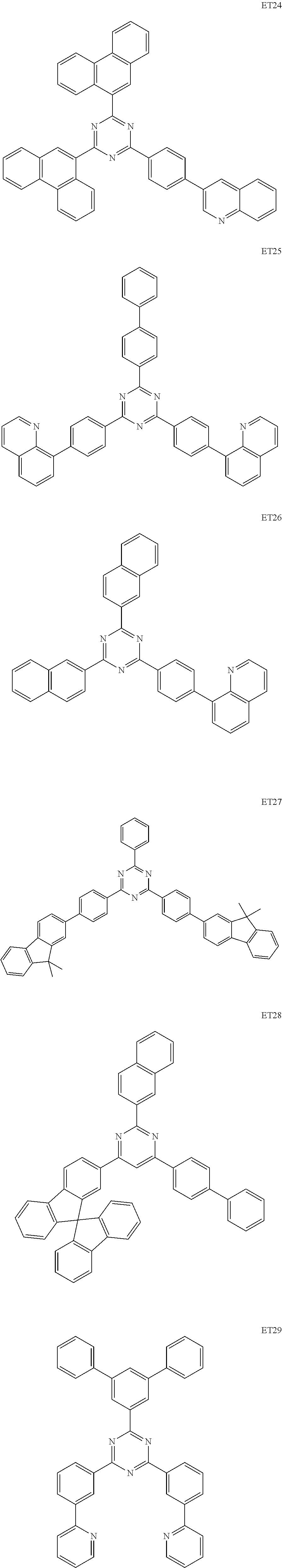

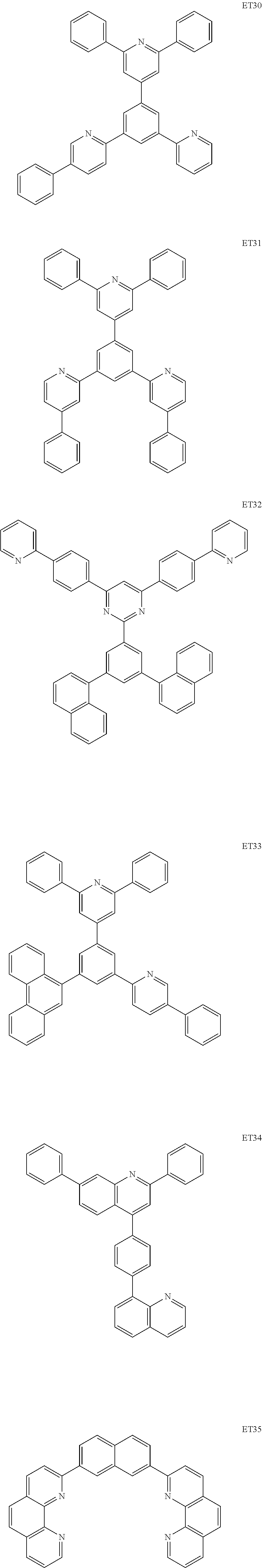 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccccc4)nc3c2)cc1 ; c1cnc2c(c1)ccc1ccc(-c3ccc4ccc(-c5ccc6ccc7cccnc7c6n5)cc4c3)nc12]

In addition, the electron transport regions ETR may include a metal halide such as LiF, NaCl, CsF, RbCl, RbI, CuI, or KI, a lanthanide metal such as Yb, and a co-deposited material of the metal halide and the lanthanide metal. For example, the electron transport region ETR may include KI:Yb, RbI:Yb, etc. as a co-deposited material. The electron transport region ETR may be formed using a metal oxide such as Li₂O or BaO, or 8-hydroxyl-lithium quinolate (Liq), etc., but the embodiments of the invention are not limited thereto. The electron transport region ETR may also be formed of a mixture material of an electron transport material and an insulating organometallic salt. The organometallic salt may be a material having an energy band gap of about 4 electron volt (eV) or more. Specifically, the organometallic salt may include, for example, metal acetates, metal benzoates, metal acetoacetates, metal acetylacetonates, or metal stearates.

The electron transport region ETR may further include at least one of 2,9-dimethyl-4,7-diphenyl-1,10-phenanthroline (BCP), or 4,7-diphenyl-1,10-phenanthroline (Bphen) in addition to the above-described materials, but the embodiments of the invention are not limited thereto. The electron transport region ETR may include the above-described compounds of the electron transport region ETR in at least one of the electron injection layer EIL, the electron transport layer ETL, or the hole blocking layer HBL.

When the electron transport region ETR includes the electron transport layer ETL, the electron transport layer ETL may have a thickness of about 100 Å to about 1,000 Å, for example, about 150 Å to about 500 Å. If the thickness of the electron transport layer ETL satisfies the aforementioned range, satisfactory electron transport characteristics may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. If the thickness of the electron injection layer EIL satisfies the above-described range, satisfactory electron injection characteristics may be obtained without a substantial increase in driving voltage.

The second electrode EL2 is provided on the electron transport region ETR. The second electrode EL2 may be a common electrode. The second electrode EL2 may be a cathode or an anode, but the embodiments are not limited thereto. For example, when the first electrode EL1 is an anode, the second electrode EL2 may be a cathode, and when the first electrode EL1 is a cathode, the second electrode EL2 may be an anode.

The second electrode EL2 may be a transmissive electrode, a transflective electrode, or a reflective electrode. When the second electrode EL2 is the transmissive electrode, the second electrode EL2 may be formed of a transparent metal oxide, for example, an indium tin oxide (ITO), an indium zinc oxide (IZO), a zinc oxide (ZnO), an indium tin zinc oxide (ITZO), etc.

When the second electrode EL2 is the transflective electrode or the reflective electrode, the second electrode EL2 may include Ag, Mg, Cu, Al, Pt, Pd, Au, Ni, Nd, Ir, Cr, L₁, Ca, LiF/Ca, LiF/Al, Mo, Ti, Yb, W, In, Zn, Sn, or a compound or mixture thereof (e.g., AgMg, AgYb, or MgAg). Alternatively, the second electrode EL2 may have a multilayer structure including a reflective film or a transflective film formed of the above-described materials, and a transparent conductive film formed of the ITO, IZO, ZnO, ITZO, etc. For example, the second electrode EL2 may include the above-described metal materials, combinations of at least two metal materials of the above-described metal materials, oxides of the above-described metal materials, or the like.

The second electrode EL2 may be connected with an auxiliary electrode. If the second electrode EL2 is connected with the auxiliary electrode, the resistance of the second electrode EL2 may decrease. A capping layer CPL may further be disposed on the second electrode EL2 of the light emitting device ED. The capping layer CPL may include a multilayer or a single layer.

In an embodiment, the capping layer CPL may be an organic layer or an inorganic layer. For example, when the capping layer CPL includes an inorganic material, the inorganic material may include an alkaline metal compound such as LiF, an alkaline earth metal compound such as MgF₂, SiON, SiNₓ, SiOy, etc.

For example, when the capping layer CPL includes an organic material, the organic material may include 2,2′-Dimethyl-N,N′-di-[(1-naphthyl)-N,N′-diphenyl]-1,1′-biphenyl-4,4′-diamine (α-NPD), NPB, TPD, m-MTDATA, Alq₃, Copper(II) phthalocyanine (CuPc), N4,N4,N4′,N4′-tetra(biphenyl-4-yl)biphenyl-4,4′-diamine (TPD15), 4,4′,4″-tris(N-carbazolyl)triphenylamine (TCTA), etc., or an epoxy resin, or acrylate such as methacrylate. However, the embodiments are not limited thereto, and the capping layer CPL may include at least one among Compounds P1 to P5 below:

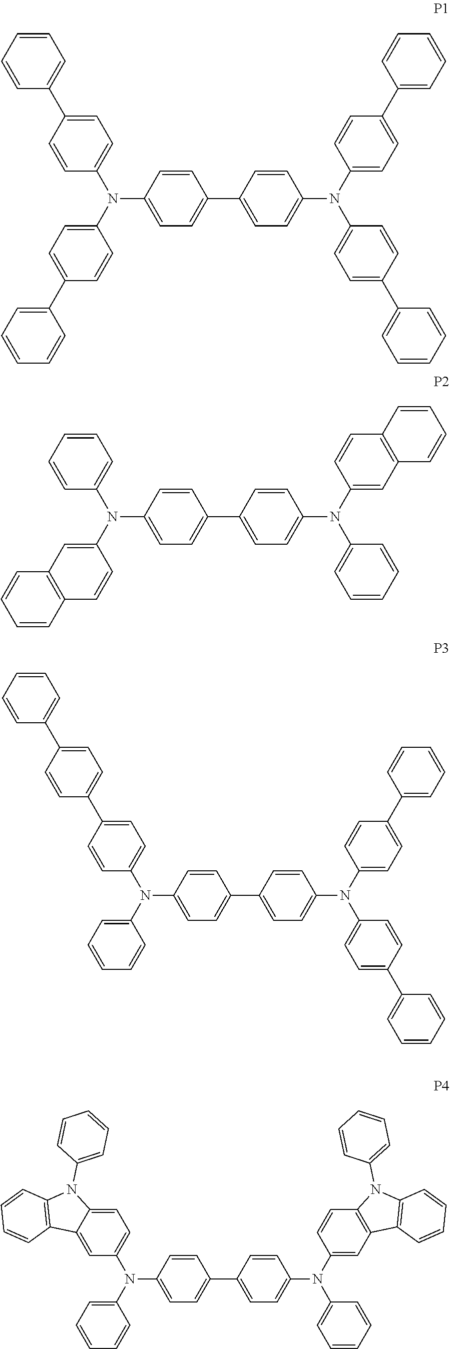 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

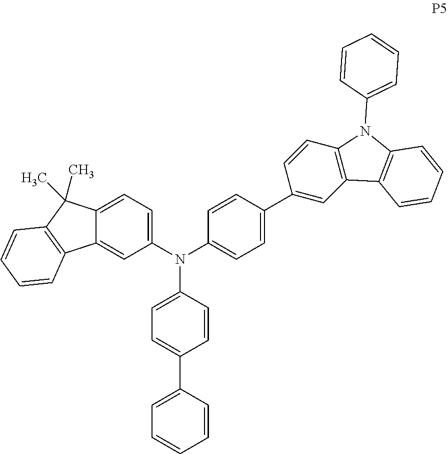 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

The refractive index of the capping layer CPL may be about 1.6 or more. Specifically, the refractive index of the capping layer CPL may be about 1.6 or more with respect to light in a wavelength range of about 550 nm to about 660 nm.

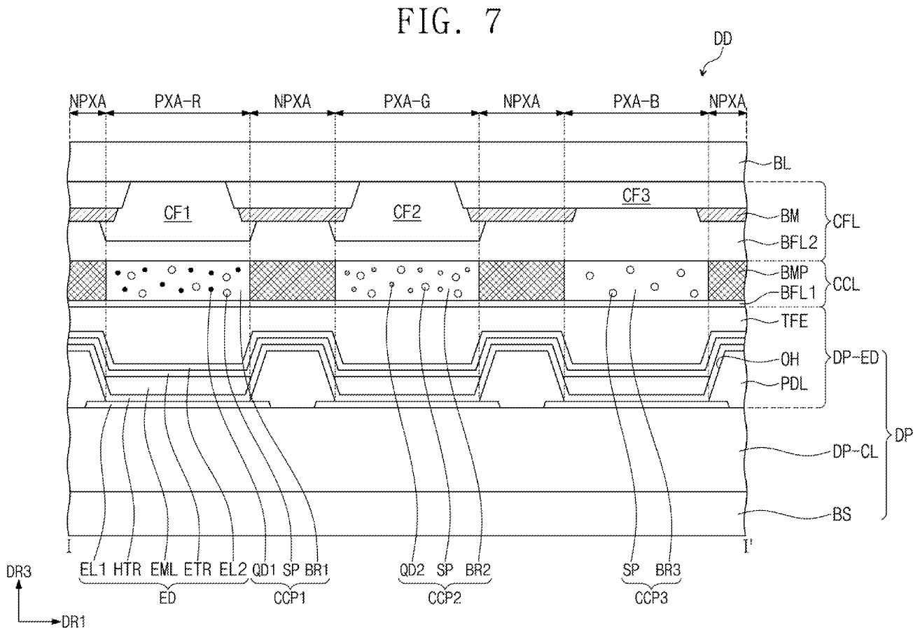

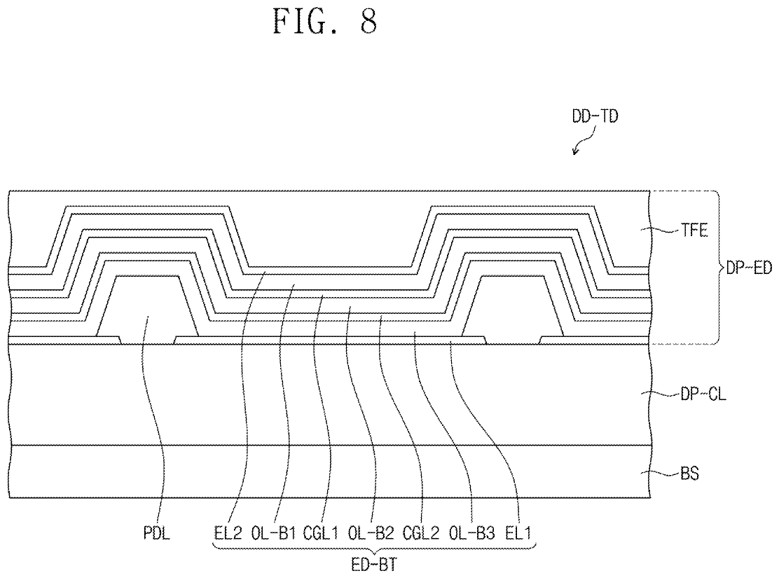

FIG. 7 is a cross-sectional view of another embodiment of a display apparatus including a light emitting device constructed according to the principles of the invention. FIG. 8 is a cross-sectional view of a further embodiment of a display apparatus including a light emitting device constructed according to the principles of the invention.

Hereinafter, in describing the display apparatus with reference to FIGS. 7 and 8, the repetitive features which have been described in FIGS. 1 to 6 are not described again, but their differences will be mainly described to avoid redundancy. Referring to FIG. 7, the display apparatus DD according to an embodiment may include a display panel DP including a display device layer DP-ED, a light control layer CCL disposed on the display panel DP, and a color filter layer CFL.

In an embodiment illustrated in FIG. 7, the display panel DP may include a base layer BS, a circuit layer DP-CL provided on the base layer BS, and the display device layer DP-ED, and the display device layer DP-ED may include a light emitting device ED. The light emitting device ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. The structures of any of the light emitting devices of FIGS. 3 to 6 as described above may be equally applied to the structure of the light emitting device ED shown in FIG. 7.

Referring to FIG. 7, the emission layer EML may be disposed in an opening OH defined in a pixel defining film PDL. For example, the emission layer EML, which is divided by the pixel defining film PDL and corresponds to each of the light emitting regions PXA-R, PXA-G, and PXA-B, may emit light in the same wavelength range. In the display apparatus DD, the emission layer EML may emit blue light. Unlike what is shown in FIG. 7, in another embodiment, the emission layer EML may be provided as a common layer in the entire light emitting regions PXA-R, PXA-G, and PXA-B.

The light control layer CCL may be disposed on the display panel DP. The light control layer CCL may include a light conversion body. The light conversion body may be a quantum dot, a phosphor, or the like. The light conversion body may emit light by converting the wavelength thereof. That is, the light control layer CCL may be a layer containing the quantum dot or a layer containing the phosphor. The light control layer CCL may include a plurality of light control parts CCP1, CCP2 and CCP3. The light control parts CCP1, CCP2, and CCP3 may be spaced apart from one another.

Referring to FIG. 7, divided patterns BMP may be disposed between the light control parts CCP1, CCP2 and CCP3 which are spaced apart from each other, but the embodiments are not limited thereto. FIG. 7 illustrates that the divided patterns BMP do not overlap the light control parts CCP1, CCP2 and CCP3, but at least a portion of the edges of the light control parts CCP1, CCP2 and CCP3 may overlap the divided patterns BMP.

The light control layer CCL may include a first light control part CCP1 containing a first quantum dot QD1 which converts first color light provided from the light emitting device ED into second color light, a second light control part CCP2 containing a second quantum dot QD2 which converts the first color light into third color light, and a third light control part CCP3 which transmits the first color light.

In an embodiment, the first light control part CCP1 may provide red light that is the second color light, and the second light control part CCP2 may provide green light that is the third color light. The third light control part CCP3 may provide blue light by transmitting the blue light that is the first color light provided in the light emitting device ED. For example, the first quantum dot QD1 may be a red quantum dot, and the second quantum dot QD2 may be a green quantum dot. The same as described above may be applied with respect to the quantum dots QD1 and QD2.

In addition, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include any quantum dot but include the scatterer SP.

The scatterer SP may be inorganic particles. For example, the scatterer SP may include at least one of a TiO₂, ZnO, Al₂O₃, SiO₂, or hollow silica. The scatterer SP may include any one of the TiO₂, ZnO, Al₂O₃, SiO₂, or hollow silica, or may be a mixture of at least two materials selected from among the TiO₂, ZnO, Al₂O₃, SiO₂, and hollow silica.

The first light control part CCP1, the second light control part CCP2, and the third light control part CCP3 each may include base resins BR1, BR2, and BR3 in which the quantum dots QD1 and QD2 and the scatterer SP are dispersed. In an embodiment, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3. The base resins BR1, BR2, and BR3 are media in which the quantum dots QD1 and QD2 and the scatterer SP are dispersed, and may be formed of various resin compositions, which may be generally referred to as a binder. For example, the base resins BR1, BR2, and BR3 may be acrylic-based resins, urethane-based resins, silicone-based resins, epoxy-based resins, etc. The base resins BR1, BR2, and BR3 may be transparent resins. In an embodiment, the first base resin BR1, the second base resin BR2, and the third base resin BR3 each may be the same as or different from each other.

The light control layer CCL may include a barrier layer BFL1. The barrier layer BFL1 may serve to prevent the penetration of moisture and/or oxygen (referred to as ‘moisture/oxygen’). The barrier layer BFL1 may be disposed on the light control units CCP1, CCP2, and CCP3 to block the light control units CCP1, CCP2 and CCP3 from being exposed to moisture/oxygen. The barrier layer BFL1 may cover the light control units CCP1, CCP2, and CCP3. In addition, the barrier layer BFL1 may be provided between the light control units CCP1, CCP2, and CCP3 and the color filter layer CFL.

The barrier layers BFL1 and BFL2 may include at least one inorganic layer. That is, the barrier layers BFL1 and BFL2 may include an inorganic material. For example, the barrier layers BFL1 and BFL2 may include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, etc. The barrier layers BFL1 and BFL2 may further include an organic film. The barrier layers BFL1 and BFL2 may be formed of a single layer or a plurality of layers.

In the display apparatus DD, the color filter layer CFL may be disposed on the light control layer CCL. For example, the color filter layer CFL may be directly disposed on the light control layer CCL. In this case, the barrier layer BFL2 may be omitted.

The color filter layer CFL may include a light shielding unit BM and filters CF1, CF2, and CF3. The color filter layer CFL may include a first filter CF1 configured to transmit the second color light, a second filter CF2 configured to transmit the third color light, and a third filter CF3 configured to transmit the first color light. For example, the first filter CF1 may be a red filter, the second filter CF2 may be a green filter, and the third filter CF3 may be a blue filter. The filters CF1, CF2, and CF3 each may include a polymeric photosensitive resin and a pigment or a dye. The first filter CF1 may include a red pigment or a dye, the second filter CF2 may include a green pigment or a dye, and the third filter CF3 may include a blue pigment or a dye. The embodiments are not limited thereto, and the third filter CF3 may not include a pigment or dye. The third filter CF3 may include a polymeric photosensitive resin and may not include a pigment or dye. The third filter CF3 may be transparent. The third filter CF3 may be formed of a transparent photosensitive resin.

Furthermore, in an embodiment, the first filter CF1 and the second filter CF2 may be a yellow filter. The first filter CF1 and the second filter CF2 may not be separated but be provided as one filter. The light shielding unit BM may be a black matrix. The light shielding unit BM may include an organic light shielding material or an inorganic light shielding material containing a black pigment or a dye. The light shielding unit BM may prevent light leakage, and may separate boundaries between the adjacent filters CF1, CF2, and CF3. In addition, in an embodiment, the light shielding unit BM may be formed of a blue filter.

The first to third filters CF1, CF2, and CF3 may be disposed corresponding to the red light emitting region PXA-R, the green light emitting region PXA-G, and the blue light emitting region PXA-B, respectively. An upper base layer BL may be disposed on the color filter layer CFL. The upper base layer BL may be a member which provides a base surface in which the color filter layer CFL, the light control layer CCL, and the like are disposed. The upper base layer BL may be a glass substrate, a metal substrate, a plastic substrate, etc. However, the embodiments are not limited thereto, and the upper base layer BL may be an inorganic layer, an organic layer, or a composite material layer. In addition, unlike shown, in an embodiment, the upper base layer BL may be omitted.

Referring to FIG. 8, in the display apparatus DD-TD, the light emitting device ED-BT may include a plurality of light emitting structures OL-B1, OL-B2, and OL-B3. The light emitting device ED-BT may include a first electrode EL1 and a second electrode EL2 which face each other, and the plurality of light emitting structures OL-B1, OL-B2, and OL-B3 sequentially stacked in the thickness direction between the first electrode EL1 and the second electrode EL2. The light emitting structures OL-B1, OL-B2, and OL-B3 each may include an emission layer EML (FIG. 7) and a hole transport region HTR and an electron transport region ETR disposed with the emission layer EML (FIG. 7) therebetween. That is, the light emitting device ED-BT included in the display apparatus DD-TD may be a light emitting device having a tandem structure and including a plurality of emission layers.

In the embodiment illustrated in FIG. 8, all light beams respectively emitted from the light emitting structures OL-B1, OL-B2, and OL-B3 may be blue light. However, the embodiments are not limited thereto, and the light beams respectively emitted from the light emitting structures OL-B1, OL-B2, and OL-B3 may have wavelength ranges different from each other. For example, the light emitting device ED-BT including the plurality of light emitting structures OL-B1, OL-B2, and OL-B3 which emit light beams having wavelength ranges different from each other may emit white light.

A charge generation layer CGL1, CGL2 may be disposed between the neighboring light emitting structures OL-B1, OL-B2, and OL-B3. The charge generation layer CGL1, CGL2 may include a p-type charge generation layer and/or an n-type charge generation layer.

The aforementioned nitrogen-containing compound may include a structure including a carbazole substituent linked to a benzofuroindole or a benzothienoindole skeleton. As the nitrogen-containing compound according to an embodiment has a structure represented by Formula 1, the light emitting device may have high efficiency and long lifespan when, although not wanting to be bound by theory, a nitrogen-containing compound made according to principles and embodiments of the invention is used as a hole transport material.

Hereinafter, with reference to Examples and Comparative Examples, a nitrogen-containing compound made according to an embodiment of the invention and a light emitting device incorporating same will be described in detail. In addition, Examples shown below are illustrated only for the understanding the principles of the invention, and the scope of the appended claims not limited thereto.

### Examples

1. Synthesis of Nitrogen-Containing Compound

First, a synthetic method of a nitrogen-containing compound according to an embodiment will be described in detail by illustrating the synthetic methods of Compounds A3, A36, A45, A50, A54, A74, B4, B99, B107, B138, B151, B263, C30, C62, C114, C148, C190, A59, C222, and C259. Also, in the following descriptions, a synthetic method of a nitrogen-containing compound is provided as an example, but the synthetic method of the nitrogen-containing compound according to an embodiment is not limited to Examples below.

(1) Synthesis of Compound A3

Synthesis of Intermediate IM-1

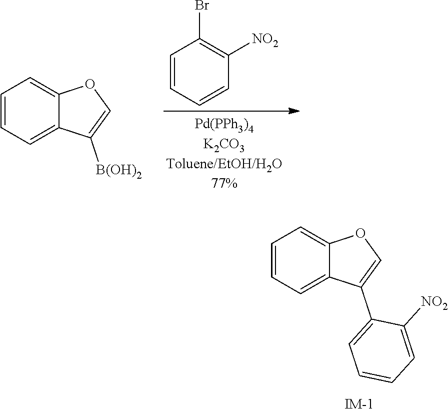

In an argon (Ar) atmosphere, in a 2000 mL three-neck flask, benzofuran-3-yl boronic acid (30.00 gram (g), 185.2 millimole (mmol)), 1-bromo-2-nitrobenzene (41.16 g, 1.1 equiv, 203.8 mmol), potassium carbonate (K₂CO₃ in an amount of 76.81 g, 3.0 equiv, 555.7 mmol), tetrakis(triphenylphosphine)palladium(0) (Pd(PPh₃)₄ in an amount of 10.70 g, 0.05 equiv, 9.3 mmol), and a mixed solution of toluene/ethanol(EtOH)/water (H₂O) in a ratio of 4/2/1; (1,297 mL) were sequentially added and heated and stirred at about 80° C. After air-cooled to room temperature, the reaction solution was extracted with toluene. A water layer was removed, and the organic layers were washed with saturated saline and then dried over magnesium sulfate (MgSO₄). The compound MgSO₄ was filtered off and the organic layers were concentrated, and then the resulting crude product was purified by silica gel column chromatography (using a mixed solvent of hexane and toluene as an eluent) to obtain Intermediate IM-1 (34.12 g, yield 77%).

By measuring fast atom bombardment/mass spectroscopy (FAB-MS), a mass number of m/z=239 was observed by molecular ion peak, thereby identifying Intermediate IM-1.

Synthesis of Intermediate IM-2

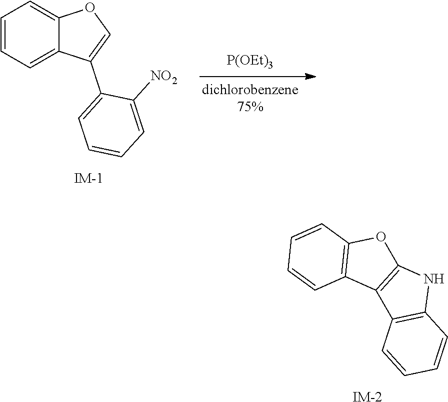

In an Ar atmosphere, in a 500 mL three-neck flask, Intermediate IM-1 (30.00 g, 125.4 mmol), o-dichlorobenzene (250 mL) and triethyl phosphite (P(OEt)₃ in an amount of 83.35 g, 4 equiv, 501.6 mmol) were sequentially added and heated and stirred at about 160° C. After the reaction solution was air-cooled to room temperature, the reaction solvent was removed by distillation, and then the resulting crude product was purified by silica gel column chromatography (using a mixed solvent of hexane and toluene as an eluent) to obtain Intermediate IM-2 (19.49 g, yield 75%).

By measuring FAB-MS, a mass number of m/z=207 was observed by molecular ion peak, thereby identifying Intermediate IM-2.

Synthesis of Intermediate IM-3

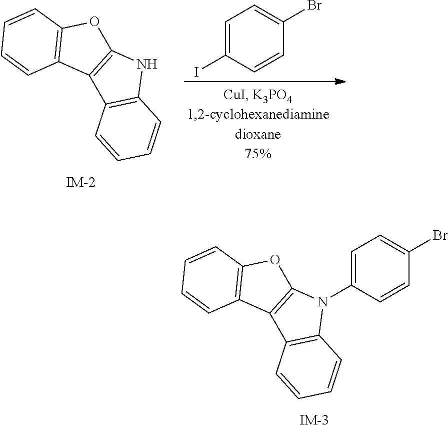

In an Ar atmosphere, in a 500 mL three-neck flask, Intermediate IM-2 (15.00 g, 72.4 mmol), copper iodide (CuI in an amount of 1.38 g, 0.1 equiv, 7.2 mmol), potassium phosphate (K₃PO₄ in an amount of 46.09 g, 3.0 equiv, 217.2 mmol), 1-bromo-4-iodobenzene (40.96 g, 2.0 equiv, 144.8 mmol), 1,4-dioxane (362 mL), and 1,2-cyclohexanediamine (1.65 g, 0.2 equiv, 14.5 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-3 (19.66 g, yield 75%).

By measuring FAB-MS, a mass number of m/z=362 was observed by molecular ion peak, thereby identifying Intermediate IM-3.

Synthesis of Compound A3

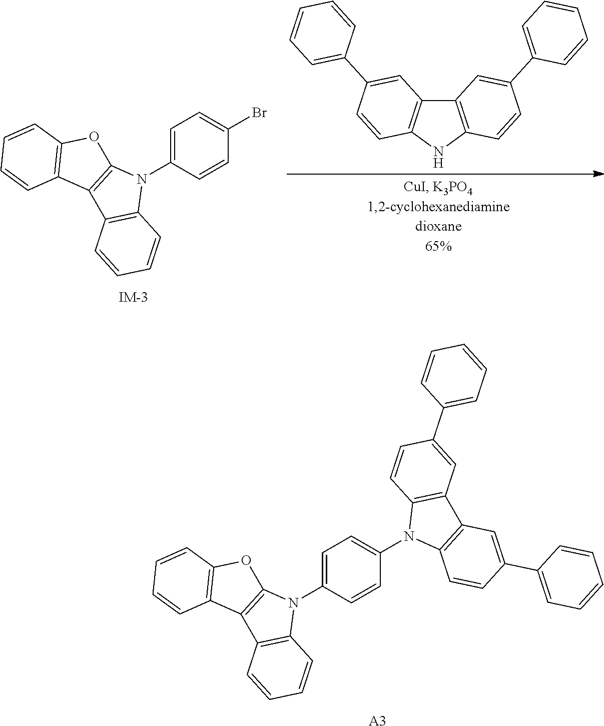

In an Ar atmosphere, in a 300 mL three-neck flask, Intermediate IM-3 (10.00 g, 37.1 mmol), CuI (0.53 g, 0.1 equiv, 2.8 mmol), K₃PO₄ (17.58 g, 3.0 equiv, 82.8 mmol), 3,6-diphenyl-9H-carbazole (8.82 g, 1.0 equiv, 27.6 mmol), 1,4-dioxane (138 mL), and 1,2-cyclohexanediamine (0.63 g, 0.2 equiv, 5.5 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Compound A3 which is solid (10.78 g, yield 65%).

By measuring FAB-MS, a mass number of m/z=600 was observed by molecular ion peak, thereby identifying Compound A3.

(2) Synthesis of Compound A36

Synthesis of Intermediate IM-4

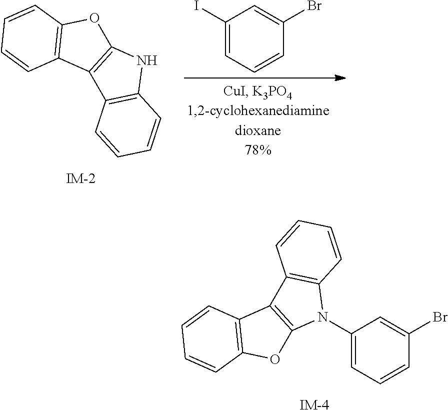

In an Ar atmosphere, in a 500 mL three-neck flask, Intermediate IM-2 (15.00 g, 72.4 mmol), CuI (1.38 g, 0.1 equiv, 7.2 mmol), K₃PO₄ (46.09 g, 3.0 equiv, 217.2 mmol), 1-bromo-3-iodobenzene (40.96 g, 2.0 equiv, 144.8 mmol), 1,4-dioxane (362 mL), and 1,2-cyclohexanediamine (1.65 g, 0.2 equiv, 14.5 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-4 (20.45 g, yield 78%).

By measuring FAB-MS, a mass number of m/z=362 was observed by molecular ion peak, thereby identifying Intermediate IM-4.

Synthesis of Compound A36

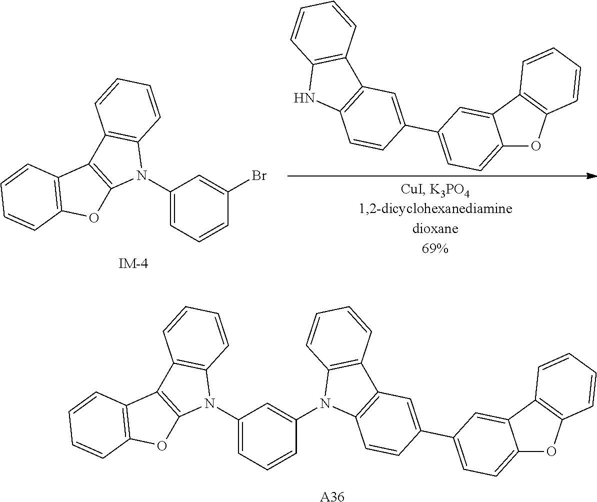

In an Ar atmosphere, in a 300 mL three-neck flask, Intermediate IM-4 (10.00 g, 27.6 mmol), CuI (0.53 g, 0.1 equiv, 2.8 mmol), K₃PO₄ (17.58 g, 3.0 equiv, 82.8 mmol), 3-(dibenzofuran-2-yl)-9H-carbazole (9.20 g, 1.0 equiv, 27.6 mmol), 1,4-dioxane (138 mL), and 1,2-cyclohexanediamine (0.63 g, 0.2 equiv, 5.5 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Compound A36 which is solid (11.71 g, yield 69%).

By measuring FAB-MS, a mass number of m/z=614 was observed by molecular ion peak, thereby identifying Compound A36.

(3) Synthesis of Compound A45

Synthesis of Intermediate IM-5

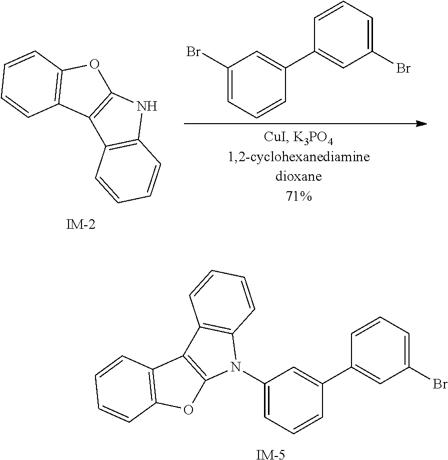

In an Ar atmosphere, in a 500 mL three-neck flask, Intermediate IM-2 (15.00 g, 72.4 mmol), CuI (1.38 g, 0.1 equiv, 7.2 mmol), K₃PO₄ (46.09 g, 3.0 equiv, 217.2 mmol), 3,3′-dibromo-1,1′-biphenyl (45.17 g, 2.0 equiv, 144.8 mmol), 1,4-dioxane (362 mL), and 1,2-cyclohexanediamine (1.65 g, 0.2 equiv, 14.5 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-5 (22.53 g, yield 71%).

By measuring FAB-MS, a mass number of m/z=438 was observed by molecular ion peak, thereby identifying Intermediate IM-5.

Synthesis of Compound A45

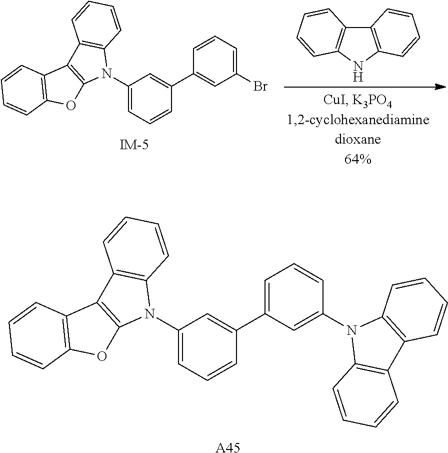

In an Ar atmosphere, in a 300 mL three-neck flask, Intermediate IM-5 (10.00 g, 22.8 mmol), CuI (0.43 g, 0.1 equiv, 2.3 mmol), K₃PO₄ (14.53 g, 3.0 equiv, 68.4 mmol), 9H-carbazole (3.81 g, 1.0 equiv, 22.8 mmol), 1,4-dioxane (115 mL), and 1,2-cyclohexanediamine (0.52 g, 0.2 equiv, 4.6 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Compound A45 which is solid (7.66 g, yield 64%).

By measuring FAB-MS, a mass number of m/z=524 was observed by molecular ion peak, thereby identifying Compound A45.

(4) Synthesis of Compound A50

Synthesis of Compound A50

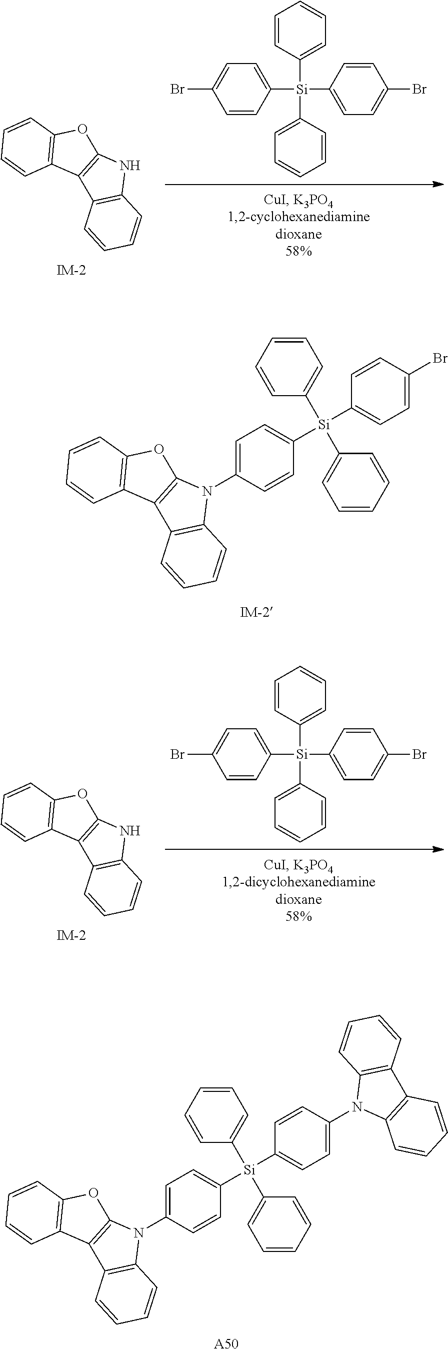

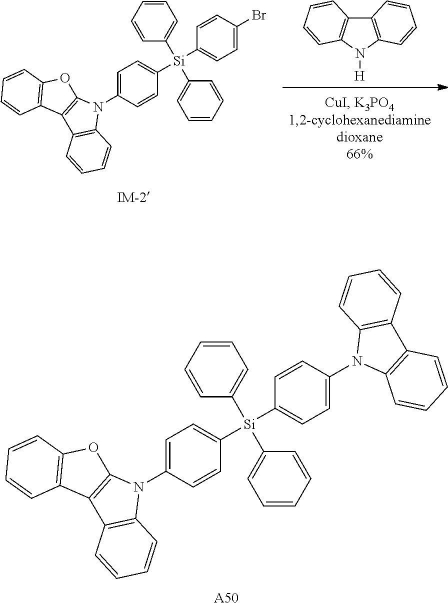 [SMILES: Brc1ccc([Si](c2ccccc2)(c2ccccc2)c2ccc(-n3c4ccccc4c4c5ccccc5oc43)cc2)cc1 ; c1ccc2c(c1)[nH]c1ccccc12 ; c1ccc([Si](c2ccccc2)(c2ccc(-n3c4ccccc4c4ccccc43)cc2)c2ccc(-n3c4ccccc4c4c5ccccc5oc43)cc2)cc1 ; "A50"]

In an Ar atmosphere, in a 500 mL three-neck flask, Intermediate IM-2 (7.49 g, 36.2 mmol), CuI (0.68 g, 0.1 equiv, 3.6 mmol), K₃PO₄ (23.0 g, 3.0 equiv, 108.5 mmol), bis(4-bromophenyl)diphenylsilane (35.7 g, 2.0 equiv, 72.2 mmol), 1,4-dioxane (181 mL), and 1,2-cyclohexanediamine (0.83 g, 0.2 equiv, 7.2 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-2′ (13.0 g, yield 58%).

In an Ar atmosphere, in a 500 mL three-neck flask, Intermediate IM-2′ (12.0 g, 19.3 mmol), CuI (0.37 g, 0.1 equiv, 1.9 mmol), K₃PO₄ (12.31 g, 3.0 equiv, 58.0 mmol), 9H-carbazole (3.23 g, 1.0 equiv, 19.3 mmol), 1,4-dioxane (97 mL), and 1,2-cyclohexanediamine (0.44 g, 0.2 equiv, 3.9 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Compound A50 (9.02 g, yield 66%).

By measuring FAB-MS, a mass number of m/z=706 was observed by molecular ion peak, thereby identifying Compound A50.

(5) Synthesis of Compound A54

Synthesis of Intermediate IM-6

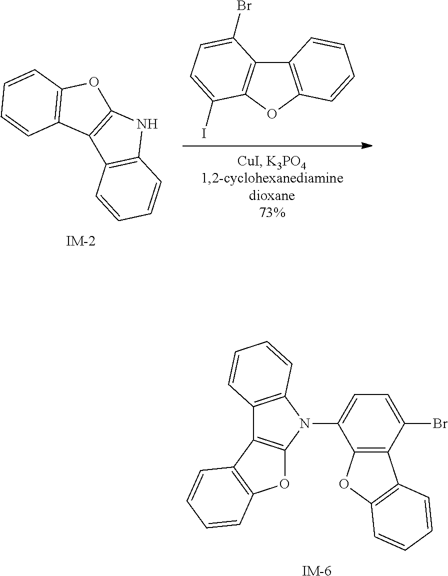

In an Ar atmosphere, in a 500 mL three-neck flask, Intermediate IM-2 (15.00 g, 72.4 mmol), CuI (1.38 g, 0.1 equiv, 7.2 mmol), K₃PO₄ (46.09 g, 3.0 equiv, 217.2 mmol), 1-bromo-4-iododibenzofuran (54.00 g, 2.0 equiv, 144.8 mmol), 1,4-dioxane (362 mL), and 1,2-cyclohexanediamine (1.65 g, 0.2 equiv, 14.5 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-6 (23.90 g, yield 73%).

By measuring FAB-MS, a mass number of m/z=452 was observed by molecular ion peak, thereby identifying Intermediate IM-6.

Synthesis of Compound A54

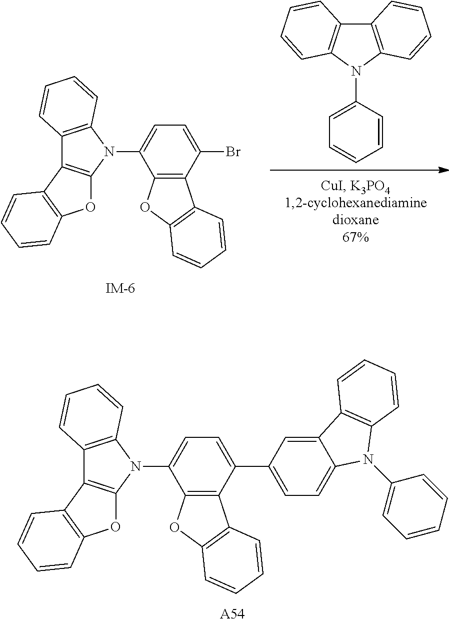

In an Ar atmosphere, in a 300 mL three-neck flask, Intermediate IM-6 (10.00 g, 22.1 mmol), CuI (0.42 g, 0.1 equiv, 2.2 mmol), K₃PO₄ (14.07 g, 3.0 equiv, 66.3 mmol), 9-phenyl-carbazole (5.37 g, 1.0 equiv, 22.1 mmol), 1,4-dioxane (110 mL), and 1,2-cyclohexanediamine (0.50 g, 0.2 equiv, 4.4 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Compound A54 which is solid (9.11 g, yield 67%).

By measuring FAB-MS, a mass number of m/z=614 was observed by molecular ion peak, thereby identifying Compound A54.

(6) Synthesis of Compound A74

Synthesis of Compound A74

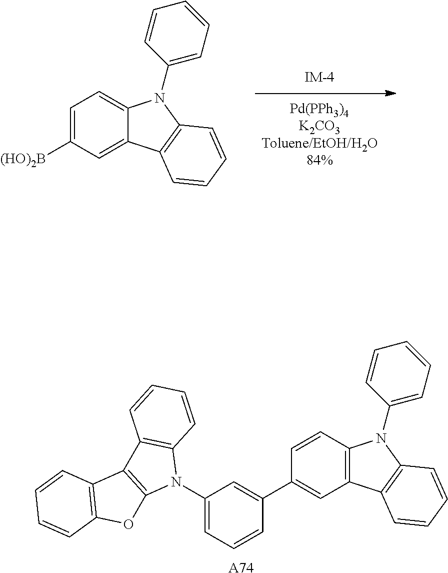

In an Ar atmosphere, in a 300 mL three-neck flask, Intermediate IM-4 (5.00 g, 13.8 mmol), (9-phenyl-9H-carbazol-3-yl)boronic acid (3.96 g, 1.0 equiv, 13.8 mmol), K₂CO₃ (5.72 g, 3.0 equiv, 41.4 mmol), Pd(PPh₃)₄ (0.80 g, 0.05 equiv, 0.7 mmol), and a mixed solution of toluene/EtOH/H₂O (4/2/1, 97 mL) were sequentially added, and then heated and stirred at 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Compound A74 (6.08 g, yield 84%).

By measuring FAB-MS, a mass number of m/z=524 was observed by molecular ion peak, thereby identifying Compound A74.

(7) Synthesis of Compound B4

Synthesis of Intermediate IM-7

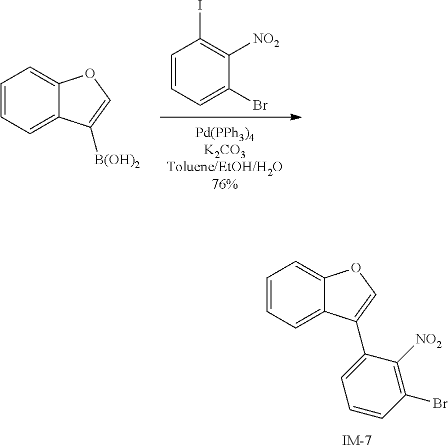

In an Ar atmosphere, in a 2000 mL three-neck flask, benzofuran-3-yl boronic acid (30.00 g, 185.2 mmol), 1-bromo-3-iodo-2-nitrobenzene (66.82 g, 1.1 equiv, 203.8 mmol), K₂CO₃ (76.81 g, 3.0 equiv, 555.7 mmol), Pd(PPh₃)₄ (10.70 g, 0.05 equiv, 9.3 mmol), and a mixed solution of toluene/EtOH/H₂O (4/2/1) (1,297 mL) were sequentially added and heated and stirred at about 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-7 (44.79 g, yield 76%).

By measuring FAB-MS, a mass number of m/z=318 was observed by molecular ion peak, thereby identifying Intermediate IM-7.

Synthesis of Intermediate IM-8

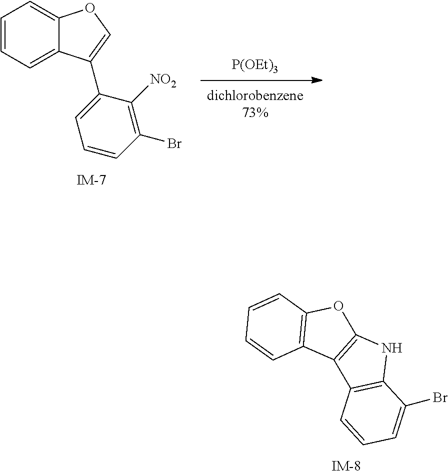

In an Ar atmosphere, in a 500 mL three-neck flask, Intermediate IM-7 (30.00 g, 94.3 mmol), o-dichlorobenzene (188 mL) and P(OEt)₃ (62.68 g, 4 equiv, 377.2 mmol) were sequentially added and heated and stirred at about 160° C. After the reaction solution was air-cooled to room temperature, the reaction solvent was removed by distillation, and then the resulting crude product was purified by silica gel column chromatography (using a mixed solvent of hexane and toluene as an eluent) to obtain Intermediate IM-8 (19.70 g, yield 73%).

By measuring FAB-MS, a mass number of m/z=286 was observed by molecular ion peak, thereby identifying Intermediate IM-8.

Synthesis of Intermediate IM-9

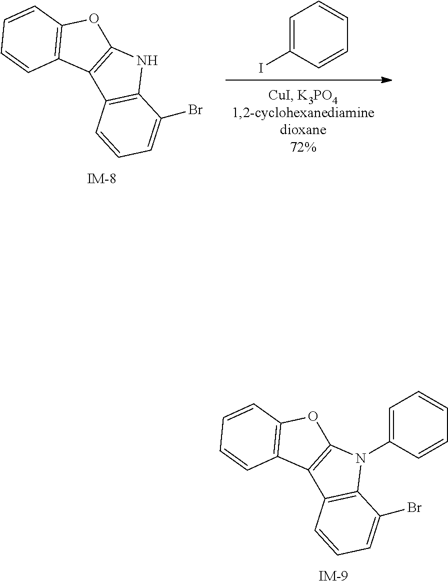

In an Ar atmosphere, in a 500 mL three-neck flask, Intermediate IM-8 (15.00 g, 52.4 mmol), CuI (1.00 g, 0.1 equiv, 5.2 mmol), K₃PO₄ (33.38 g, 3.0 equiv, 157.3 mmol), iodobenzene (21.39 g, 2.0 equiv, 104.8 mmol), 1,4-dioxane (262 mL), and 1,2-cyclohexanediamine (1.20 g, 0.2 equiv, 10.5 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-9 (13.67 g, yield 72%).

By measuring FAB-MS, a mass number of m/z=362 was observed by molecular ion peak, thereby identifying Intermediate IM-9.

Synthesis of Intermediate IM-10

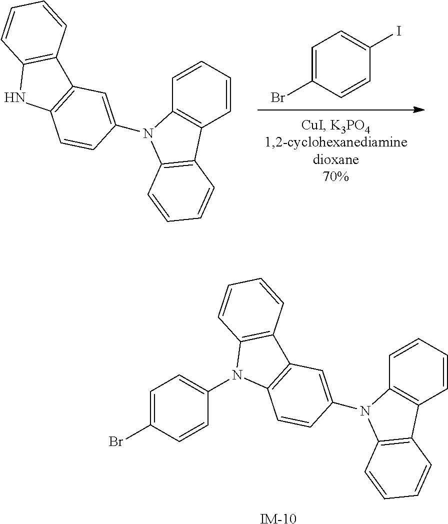

In an Ar atmosphere, in a 500 mL three-neck flask, 9H-3,9′-bicarbazole (20.00 g, 60.2 mmol), CuI (1.15 g, 0.1 equiv, 6.0 mmol), K₃PO₄ (38.81 g, 3.0 equiv, 180.5 mmol), 1-bromo-4-iodobenzene (85.11 g, 5.0 equiv, 300.8 mmol), 1,4-dioxane (300 mL), and 1,2-cyclohexanediamine (1.37 g, 0.2 equiv, 12.0 mmol) were sequentially added and heated and stirred under reflux. After the reaction solution was air-cooled to room temperature, the organic layer was fractionated by adding water to the reaction solvent and performing a suction filtration with celite. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-10 (21.88 g, yield 70%).

By measuring FAB-MS, a mass number of m/z=519 was observed by molecular ion peak, thereby identifying Intermediate IM-10.

Synthesis of Intermediate IM-11

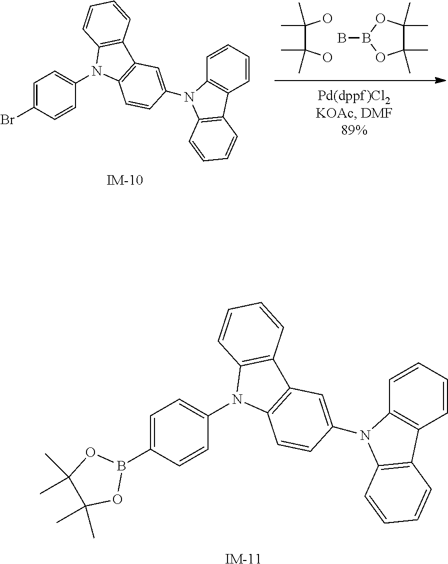

In an Ar atmosphere, in a 300 mL three-neck flask, Intermediate IM-10 (15.00 g, 28.9 mmol), [1,1′-bis(diphenylphosphino)ferrocene]dichloropalladium(II) (Pd(dppf)Cl₂ in an amount of 2.39 g, 0.1 equiv, 2.9 mmol), potassium acetate (KOAc in an amount of 5.67 g, 2.0 equiv, 57.7 mmol), dimethylformamide (DMF in an amount of 144 mL) and bis(pinacolato)dibron (8.80 g, 1.2 equiv, 34.6 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-11 (14.56 g, yield 89%).

By measuring FAB-MS, a mass number of m/z=566 was observed by molecular ion peak, thereby identifying Intermediate IM-11.

Synthesis of Compound B4

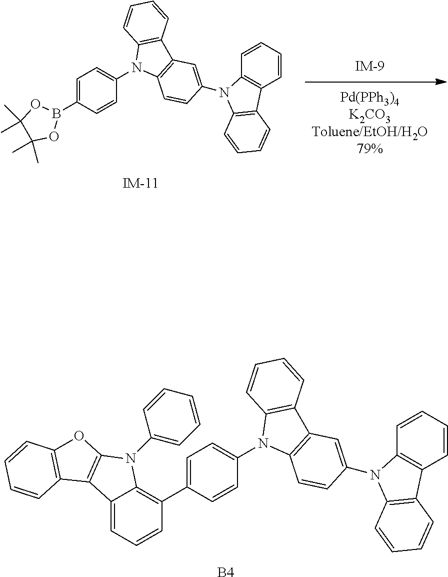

In an Ar atmosphere, in a 300 mL three-neck flask, Intermediate IM-9 (5.00 g, 13.8 mmol), Intermediate IM-11 (7.82 g, 1.0 equiv, 13.8 mmol), K₂CO₃ (5.72 g, 3.0 equiv, 41.4 mmol), Pd(PPh₃)₄ (0.80 g, 0.05 equiv, 0.7 mmol), and a mixed solution of toluene/EtOH/H₂O (4/2/1) (97 mL) were sequentially added and heated and stirred at about 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Compound B4 (7.52 g, yield 79%).

By measuring FAB-MS, a mass number of m/z=689 was observed by molecular ion peak, thereby identifying Compound B4.

(8) Synthesis of Compound B99

Synthesis of Intermediate IM-12

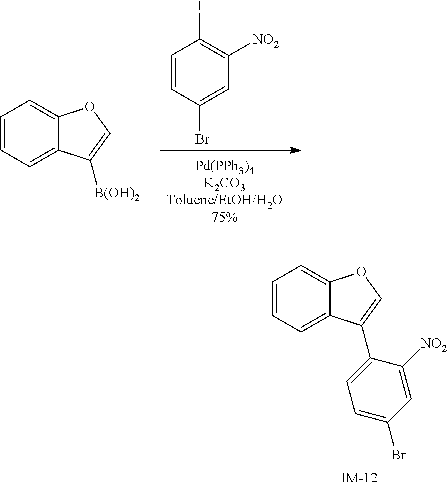

In an Ar atmosphere, in a 2000 mL three-neck flask, benzofuran-3-yl boronic acid (30.00 g, 185.2 mmol), 4-bromo-1-iodo-2-nitrobenzene (66.82 g, 1.1 equiv, 203.8 mmol), K₂CO₃ (76.81 g, 3.0 equiv, 555.7 mmol), Pd(PPh₃)₄ (10.70 g, 0.05 equiv, 9.3 mmol), and a mixed solution of toluene/EtOH/H₂O (4/2/1) (1,297 mL) were sequentially added and heated and stirred at about 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-12 (44.20 g, yield 75%).

By measuring FAB-MS, a mass number of m/z=318 was observed by molecular ion peak, thereby identifying Intermediate IM-12.

Synthesis of Intermediate IM-13

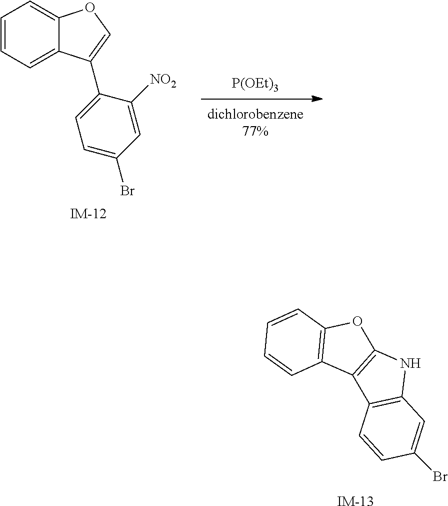

In an Ar atmosphere, in a 500 mL three-neck flask, Intermediate IM-12 (30.00 g, 94.3 mmol), o-dichlorobenzene (188 mL) and P(OEt)₃ (62.68 g, 4 equiv, 377.2 mmol) were sequentially added and heated and stirred at about 160° C. After the reaction solution was air-cooled to room temperature, the reaction solvent was removed by distillation, and then the resulting crude product was purified by silica gel column chromatography (using a mixed solvent of hexane and toluene as an eluent) to obtain Intermediate IM-13 (20.78 g, yield 77%).

By measuring FAB-MS, a mass number of m/z=286 was observed by molecular ion peak, thereby identifying Intermediate IM-13.

Synthesis of Intermediate IM-14

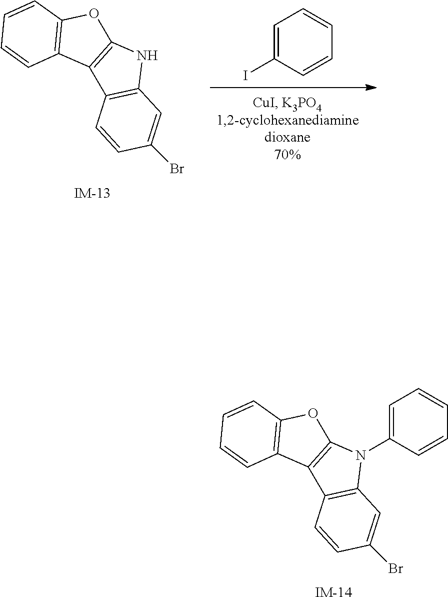

In an Ar atmosphere, in a 300 mL three-neck flask, Intermediate IM-13 (15.00 g, 52.4 mmol), CuI (1.00 g, 0.1 equiv, 5.2 mmol), K₃PO₄ (33.38 g, 3.0 equiv, 157.3 mmol), iodobenzene (21.39 g, 2.0 equiv, 104.8 mmol), 1,4-dioxane (262 mL), and 1,2-cyclohexanediamine (1.20 g, 0.2 equiv, 10.5 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-14 (13.29 g, yield 70%).

By measuring FAB-MS, a mass number of m/z=362 was observed by molecular ion peak, thereby identifying Intermediate IM-14.

Synthesis of Intermediate IM-15

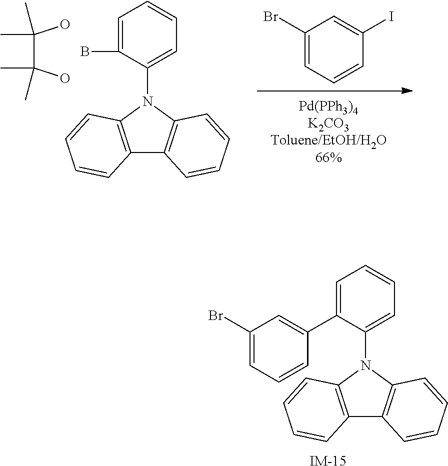

In an Ar atmosphere, in a 1000 mL three-neck flask, 9-[2-(4,4,5,5-tetramethyl-1,3,2-dioxaborolan-2-yl)phenyl]-9H-carbazole (25.00 g, 67.7 mmol), 1-bromo-3-iodobenzene (19.15 g, 1.1 equiv, 67.7 mmol), K₂CO₃ (28.07 g, 3.0 equiv, 203.1 mmol), Pd(PPh₃)₄ (3.91 g, 0.05 equiv, 3.4 mmol), and a mixed solution of toluene/EtOH/H₂O (4/2/1) (473 mL) were sequentially added and heated and stirred at about 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-15 (17.80 g, yield 66%).

By measuring FAB-MS, a mass number of m/z=398 was observed by molecular ion peak, thereby identifying Intermediate IM-15.

Synthesis of Intermediate IM-16

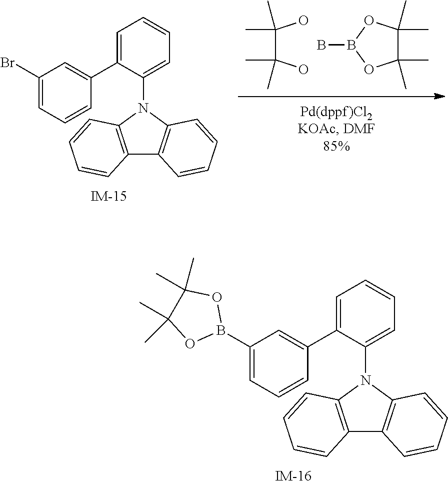

In an Ar atmosphere, in a 300 mL three-neck flask, Intermediate IM-15 (15.00 g, 37.7 mmol), Pd(dppf)Cl₂ (3.08 g, 0.1 equiv, 3.8 mmol), KOAc (7.39 g, 2.0 equiv, 75.3 mmol), DMF (188 mL) and bis(pinacolato)dibron (11.48 g, 1.2 equiv, 45.2 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-16 (14.26 g, yield 85%).

By measuring FAB-MS, a mass number of m/z=445 was observed by molecular ion peak, thereby identifying Intermediate IM-16.

Synthesis of Compound B99

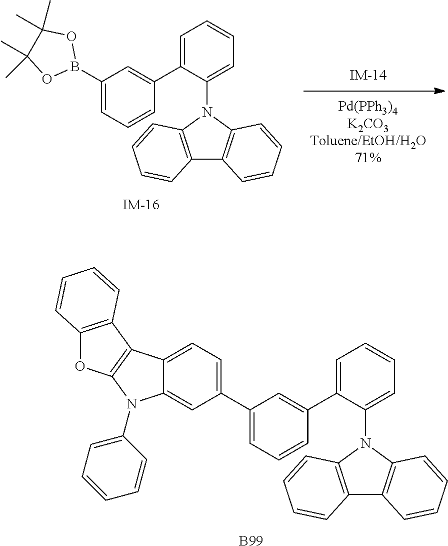

In an Ar atmosphere, in a 300 mL three-neck flask, Intermediate IM-14 (5.00 g, 13.8 mmol), Intermediate IM-16 (6.15 g, 1.0 equiv, 13.8 mmol), K₂CO₃ (5.72 g, 3.0 equiv, 41.4 mmol), Pd(PPh₃)₄ (0.80 g, 0.05 equiv, 0.7 mmol), and a mixed solution of toluene/EtOH/H₂O (4/2/1) (97 mL) were sequentially added and heated and stirred at about 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Compound B99 (5.89 g, yield 71%).

By measuring FAB-MS, a mass number of m/z=600 was observed by molecular ion peak, thereby identifying Compound B99.

(9) Synthesis of Compound B107

Synthesis of Intermediate IM-17

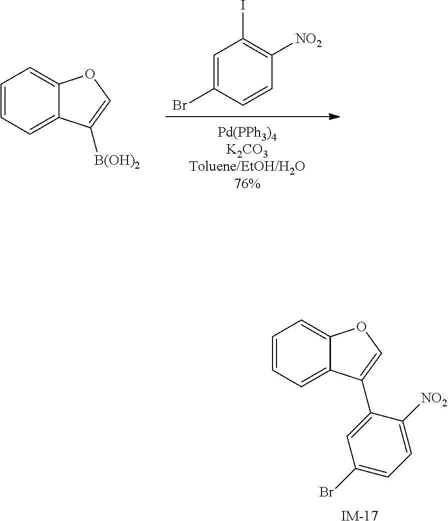

In an Ar atmosphere, in a 2000 mL three-neck flask, benzofuran-3-yl boronic acid (30.00 g, 185.2 mmol), 4-bromo-2-iodo-1-nitrobenzene (66.82 g, 1.1 equiv, 203.8 mmol), K₂CO₃ (76.81 g, 3.0 equiv, 555.7 mmol), Pd(PPh₃)₄ (10.70 g, 0.05 equiv, 9.3 mmol), and a mixed solution of toluene/EtOH/H₂O (4/2/1) (1,297 mL) were sequentially added and heated and stirred at about 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-17 (44.79 g, yield 76%).

By measuring FAB-MS, a mass number of m/z=318 was observed by molecular ion peak, thereby identifying Intermediate IM-17.

Synthesis of Intermediate IM-18

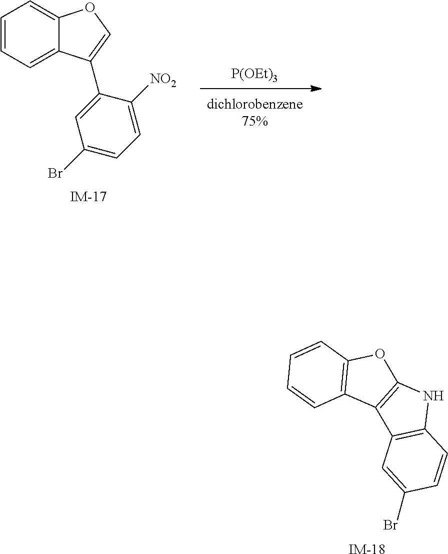

In an Ar atmosphere, in a 500 mL three-neck flask, Intermediate IM-17 (30.00 g, 94.3 mmol), o-dichlorobenzene (188 mL) and P(OEt)₃ (62.68 g, 4 equiv, 377.2 mmol) were sequentially added and heated and stirred at about 160° C. After the reaction solution was air-cooled to room temperature, the reaction solvent was removed by distillation, and then the resulting crude product was purified by silica gel column chromatography (using a mixed solvent of hexane and toluene as an eluent) to obtain Intermediate IM-18 (20.24 g, yield 75%).

By measuring FAB-MS, a mass number of m/z=286 was observed by molecular ion peak, thereby identifying Intermediate IM-18.

Synthesis of Intermediate IM-19

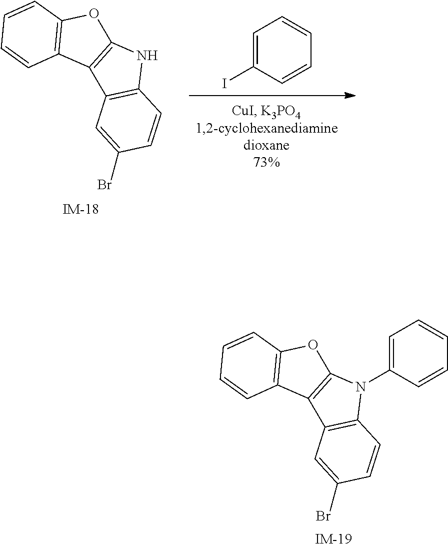

In an Ar atmosphere, in a 500 mL three-neck flask, Intermediate IM-18 (15.00 g, 52.4 mmol), CuI (1.00 g, 0.1 equiv, 5.2 mmol), K₃PO₄ (33.38 g, 3.0 equiv, 157.3 mmol), iodobenzene (21.39 g, 2.0 equiv, 104.8 mmol), 1,4-dioxane (262 mL), and 1,2-cyclohexanediamine (1.20 g, 0.2 equiv, 10.5 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-19 (13.86 g, yield 73%).

By measuring FAB-MS, a mass number of m/z=362 was observed by molecular ion peak, thereby identifying Intermediate IM-19.

Synthesis of Intermediate IM-20

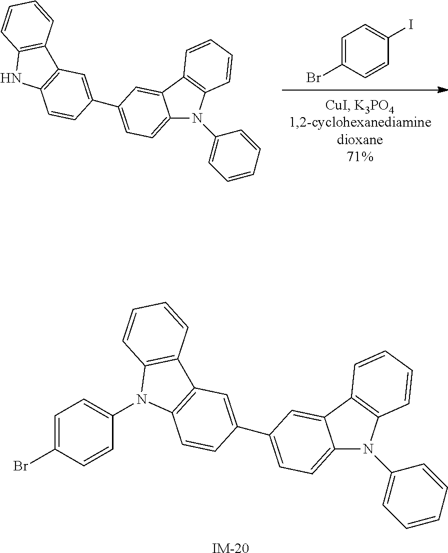

In an Ar atmosphere, in a 500 mL three-neck flask, 9-phenyl-9H,9′H-3,3′-bicarbazole (20.00 g, 49.0 mmol), CuI (0.93 g, 0.1 equiv, 4.9 mmol), K₃PO₄ (31.18 g, 3.0 equiv, 146.9 mmol), 1-bromo-4-iodobenzene (69.26 g, 5.0 equiv, 244.8 mmol), 1,4-dioxane (244 mL), and 1,2-cyclohexanediamine (1.12 g, 0.2 equiv, 9.8 mmol) were sequentially added and heated and stirred under reflux. After the reaction solution was air-cooled to room temperature, the organic layer was fractionated by adding water to the reaction solvent and performing a suction filtration with celite. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-20 (20.14 g, yield 73%).

By measuring FAB-MS, a mass number of m/z=519 was observed by molecular ion peak, thereby identifying Intermediate IM-20.

Synthesis of Intermediate IM-21

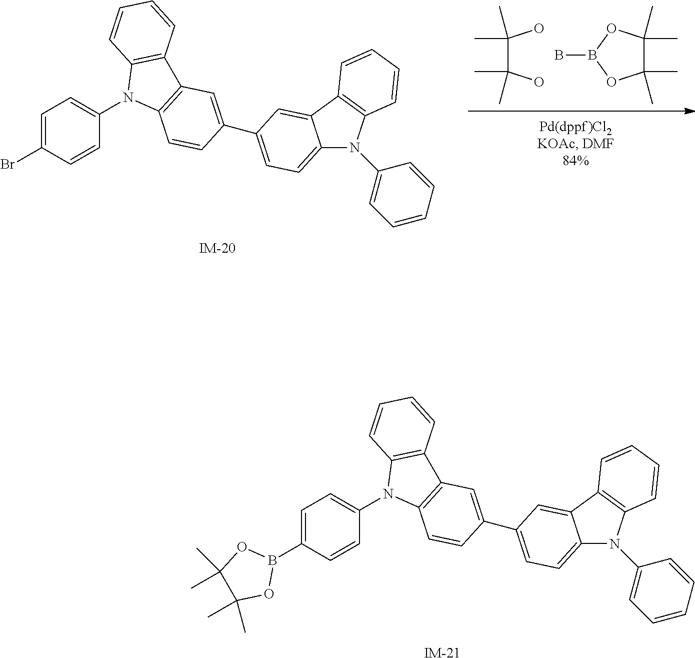

In an Ar atmosphere, in a 300 mL three-neck flask, Intermediate IM-20 (15.00 g, 26.6 mmol), Pd(dppf)Cl₂ (2.17 g, 0.1 equiv, 2.7 mmol), KOAc (5.22 g, 2.0 equiv, 53.2 mmol), DMF (144 mL) and bis(pinacolato)dibron (8.11 g, 1.2 equiv, 31.9 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-21 (13.65 g, yield 84%).

By measuring FAB-MS, a mass number of m/z=610 was observed by molecular ion peak, thereby identifying Intermediate IM-21.

Synthesis of Compound B107

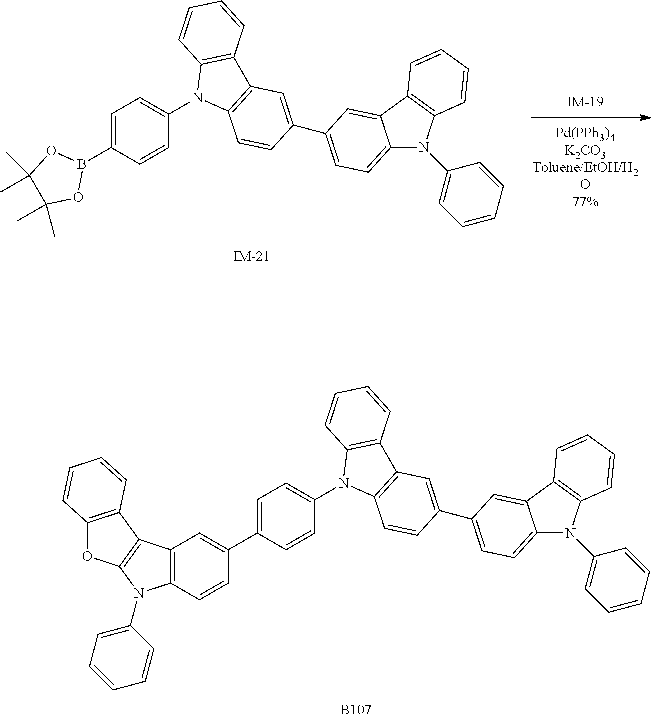

In an Ar atmosphere, in a 300 mL three-neck flask, Intermediate IM-19 (5.00 g, 13.8 mmol), Intermediate IM-21 (8.87 g, 1.0 equiv, 13.8 mmol), K₂CO₃ (5.72 g, 3.0 equiv, 41.4 mmol), Pd(PPh₃)₄ (0.80 g, 0.05 equiv, 0.7 mmol), and a mixed solution of toluene/EtOH/H₂O (4/2/1) (97 mL) were sequentially added and heated and stirred at about 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Compound B107 (8.46 g, yield 77%).

By measuring FAB-MS, a mass number of m/z=765 was observed by molecular ion peak, thereby identifying Compound B107.

(10) Synthesis of Compound B138

Synthesis of Intermediate IM-22

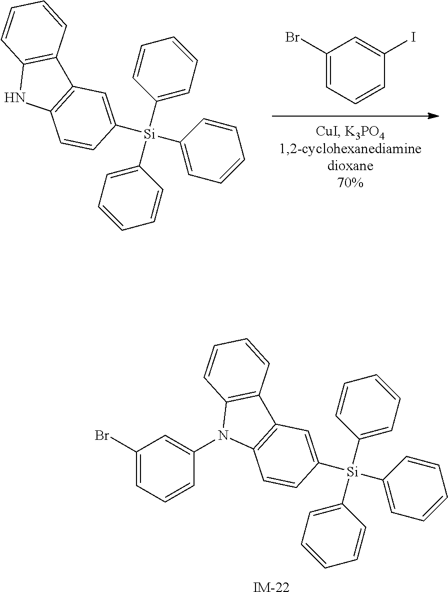

In an Ar atmosphere, in a 500 mL three-neck flask, 3-(triphenylsilyl)-9H-carbazole (20.00 g, 47.0 mmol), CuI (0.89 g, 0.1 equiv, 4.7 mmol), K₃PO₄ (29.92 g, 3.0 equiv, 140.9 mmol), 1-bromo-3-iodobenzene (66.47 g, 5.0 equiv, 235.0 mmol), 1,4-dioxane (235 mL), and 1,2-cyclohexanediamine (1.07 g, 0.2 equiv, 9.40 mmol) were sequentially added and heated and stirred under reflux. After the reaction solution was air-cooled to room temperature, the organic layer was fractionated by adding water to the reaction solvent and performing a suction filtration with celite. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-22 (19.10 g, yield 70%).

By measuring FAB-MS, a mass number of m/z=580 was observed by molecular ion peak, thereby identifying Intermediate IM-22.

Synthesis of Intermediate IM-23

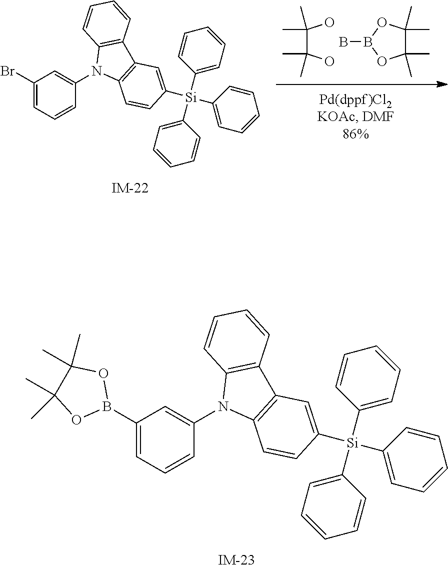

In an Ar atmosphere, in a 300 mL three-neck flask, Intermediate IM-22 (15.00 g, 25.8 mmol), Pd(dppf)Cl₂ (2.11 g, 0.1 equiv, 2.6 mmol), KOAc (5.07 g, 2.0 equiv, 51.7 mmol), DMF (130 mL) and bis(pinacolato)dibron (7.87 g, 1.2 equiv, 31.0 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-23 (13.95 g, yield 86%).

By measuring FAB-MS, a mass number of m/z=627 was observed by molecular ion peak, thereby identifying Intermediate IM-23.

Synthesis of Compound B138

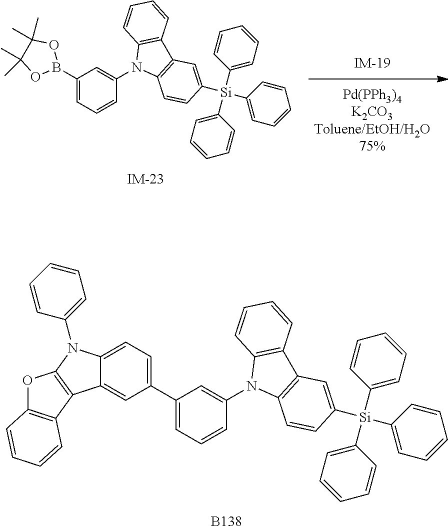

In an Ar atmosphere, in a 300 mL three-neck flask, Intermediate IM-19 (5.00 g, 13.8 mmol), Intermediate IM-23 (8.66 g, 1.0 equiv, 13.8 mmol), K₂CO₃ (5.72 g, 3.0 equiv, 41.4 mmol), Pd(PPh₃)₄ (0.80 g, 0.05 equiv, 0.7 mmol), and a mixed solution of toluene/EtOH/H₂O (4/2/1) (97 mL) were sequentially added and heated and stirred at about 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Compound B138 (8.11 g, yield 75%).

By measuring FAB-MS, a mass number of m/z=783 was observed by molecular ion peak, thereby identifying Compound B138.

(11) Synthesis of Compound B151

Synthesis of Intermediate IM-24

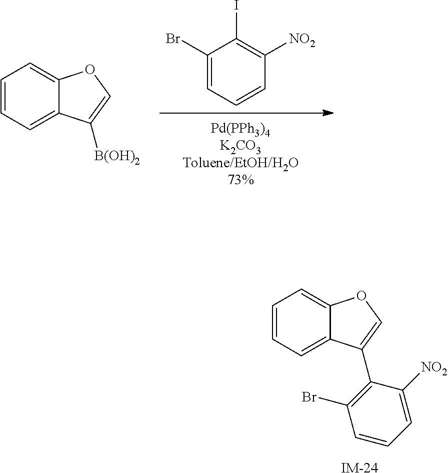

In an Ar atmosphere, in a 2000 mL three-neck flask, benzofuran-3-yl boronic acid (30.00 g, 185.2 mmol), 1-bromo-2-iodo-3-nitrobenzene (66.82 g, 1.1 equiv, 203.8 mmol), K₂CO₃ (76.81 g, 3.0 equiv, 555.7 mmol), Pd(PPh₃)₄ (10.70 g, 0.05 equiv, 9.3 mmol), and a mixed solution of toluene/EtOH/H₂O (4/2/1) (1,297 mL) were sequentially added and heated and stirred at about 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-24 (43.02 g, yield 73%).

By measuring FAB-MS, a mass number of m/z=318 was observed by molecular ion peak, thereby identifying Intermediate IM-24.

Synthesis of Intermediate IM-25

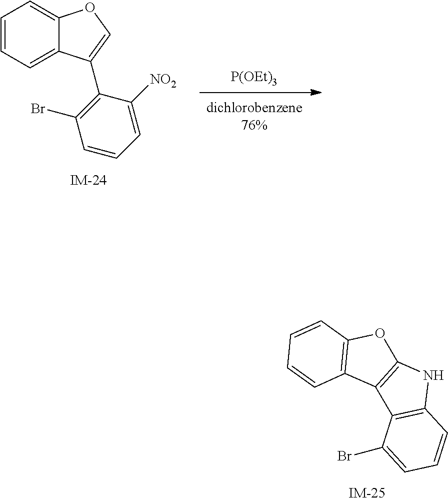

In an Ar atmosphere, in a 500 mL three-neck flask, Intermediate IM-24 (30.00 g, 94.3 mmol), o-dichlorobenzene (188 mL) and P(OEt)₃ (62.68 g, 4 equiv, 377.2 mmol) were sequentially added and heated and stirred at about 160° C. After the reaction solution was air-cooled to room temperature, the reaction solvent was removed by distillation, and then the resulting crude product was purified by silica gel column chromatography (using a mixed solvent of hexane and toluene as an eluent) to obtain Intermediate IM-25 (20.51 g, yield 76%).

By measuring FAB-MS, a mass number of m/z=286 was observed by molecular ion peak, thereby identifying Intermediate IM-25.

Synthesis of Intermediate IM-26

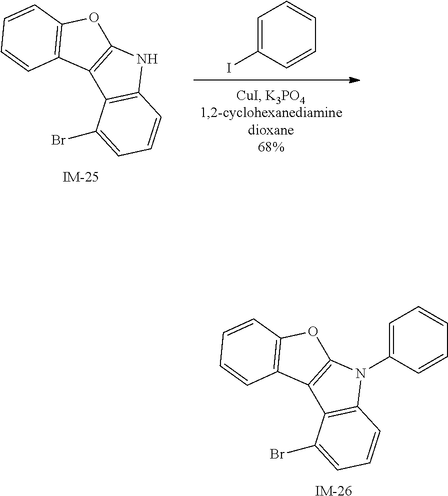

In an Ar atmosphere, in a 500 mL three-neck flask, Intermediate IM-25 (15.00 g, 52.4 mmol), CuI (1.00 g, 0.1 equiv, 5.2 mmol), K₃PO₄ (33.38 g, 3.0 equiv, 157.3 mmol), iodobenzene (21.39 g, 2.0 equiv, 104.8 mmol), 1,4-dioxane (262 mL), and 1,2-cyclohexanediamine (1.20 g, 0.2 equiv, 10.5 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-26 (12.91 g, yield 68%).

By measuring FAB-MS, a mass number of m/z=362 was observed by molecular ion peak, thereby identifying Intermediate IM-26.

Synthesis of Compound B151

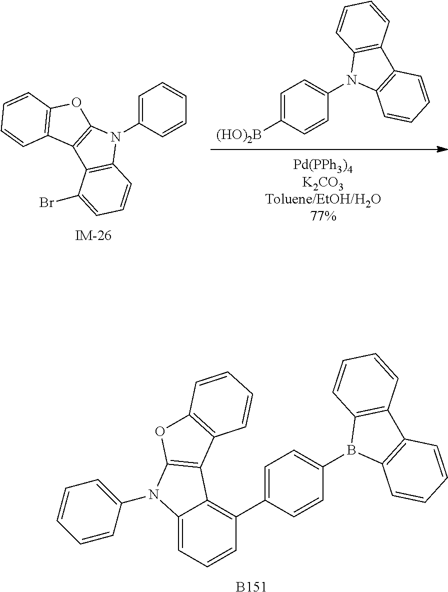

In an Ar atmosphere, in a 300 mL three-neck flask, Intermediate IM-26 (5.00 g, 13.8 mmol), [4-(9H-carbazol-9-yl)phenyl)]boronic acid (3.96 g, 1.0 equiv, 13.8 mmol), K₂CO₃ (5.72 g, 3.0 equiv, 41.4 mmol), Pd(PPh₃)₄ (0.80 g, 0.05 equiv, 0.7 mmol), and a mixed solution of toluene/EtOH/H₂O (4/2/1, 97 mL) were sequentially added, and then heated and stirred at 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Compound B151 (5.58 g, yield 77%).

By measuring FAB-MS, a mass number of m/z=524 was observed by molecular ion peak, thereby identifying Compound B151.

(12) Synthesis of Compound B263

Synthesis of Compound B263

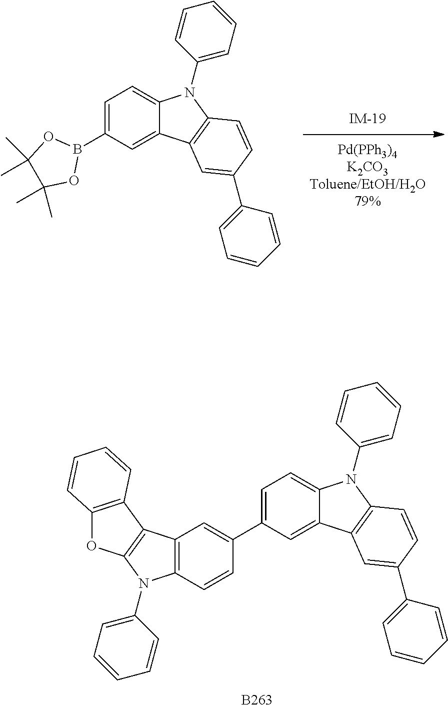 [SMILES: CC1(C)OB(c2ccc3c(c2)c2cc(-c4ccccc4)ccc2n3-c2ccccc2)OC1(C)C ; c1ccc(-c2ccc3c(c2)c2cc(-c4ccc5c(c4)c4c6ccccc6oc4n5-c4ccccc4)ccc2n3-c2ccccc2)cc1 ; "B263"]

In an Ar atmosphere, in a 300 mL three-neck flask, Intermediate IM-19 (5.00 g, 13.8 mmol), 3,9-diphenyl-6-(4,4,5,5-tetramethyl-1,3,2-dioxaborolan-2-yl)-9H-carbazole (6.15 g, 1.0 equiv, 13.8 mmol), K₂CO₃ (5.72 g, 3.0 equiv, 41.4 mmol), Pd(PPh₃)₄ (0.80 g, 0.05 equiv, 0.7 mmol), and a mixed solution of toluene/EtOH/H₂O (4/2/1, 97 mL) were sequentially added, and then heated and stirred at 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Compound B263 (6.55 g, yield 79%).

By measuring FAB-MS, a mass number of m/z=600 was observed by molecular ion peak, thereby identifying Compound B263.

(13) Synthesis of Compound C30

Synthesis of Intermediate IM-27

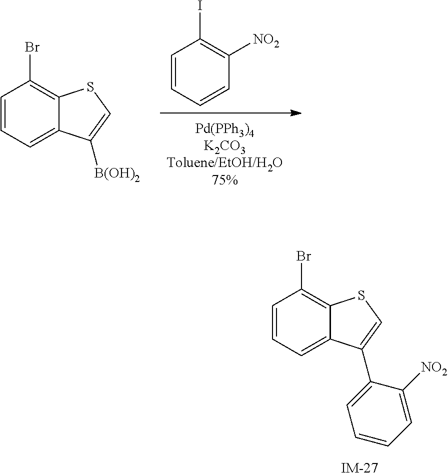

In an Ar atmosphere, in a 2000 mL three-neck flask, (7-bromobenzothiophene-3-yl)boronic acid (35.00 g, 136.2 mmol), 1-iodo-2-nitrobenzene (37.32 g, 1.1 equiv, 149.9 mmol), K₂CO₃ (56.48 g, 3.0 equiv, 408.7 mmol), Pd(PPh₃)₄ (7.87 g, 0.05 equiv, 6.8 mmol), and a mixed solution of toluene/EtOH/H₂O (4/2/1) (954 mL) were sequentially added and heated and stirred at about 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-27 (34.15 g, yield 75%).

By measuring FAB-MS, a mass number of m/z=334 was observed by molecular ion peak, thereby identifying Intermediate IM-27.

Synthesis of Intermediate IM-28

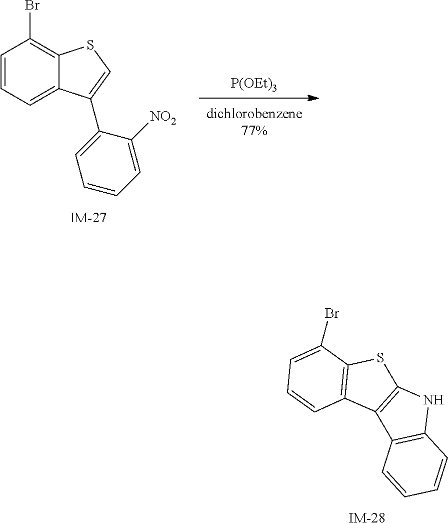

In an Ar atmosphere, in a 500 mL three-neck flask, Intermediate IM-27 (30.00 g, 89.8 mmol), o-dichlorobenzene (180 mL) and P(OEt)₃ (59.66 g, 4 equiv, 359.1 mmol) were sequentially added and heated and stirred at about 160° C. After the reaction solution was air-cooled to room temperature, the reaction solvent was removed by distillation, and then the resulting crude product was purified by silica gel column chromatography (using a mixed solvent of hexane and toluene as an eluent) to obtain Intermediate IM-28 (20.89 g, yield 77%).

By measuring FAB-MS, a mass number of m/z=302 was observed by molecular ion peak, thereby identifying Intermediate IM-28.

Synthesis of Intermediate IM-29

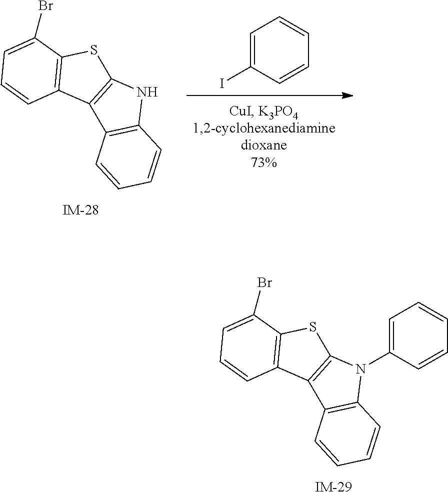

In an Ar atmosphere, in a 500 mL three-neck flask, Intermediate IM-28 (15.00 g, 49.6 mmol), CuI (0.94 g, 0.1 equiv, 5.0 mmol), K₃PO₄ (31.61 g, 3.0 equiv, 148.9 mmol), iodobenzene (20.25 g, 2.0 equiv, 99.3 mmol), 1,4-dioxane (248 mL), and 1,2-cyclohexanediamine (1.13 g, 0.2 equiv, 9.9 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-29 (13.71 g, yield 73%).

By measuring FAB-MS, a mass number of m/z=378 was observed by molecular ion peak, thereby identifying Intermediate IM-29.

Synthesis of Intermediate IM-30

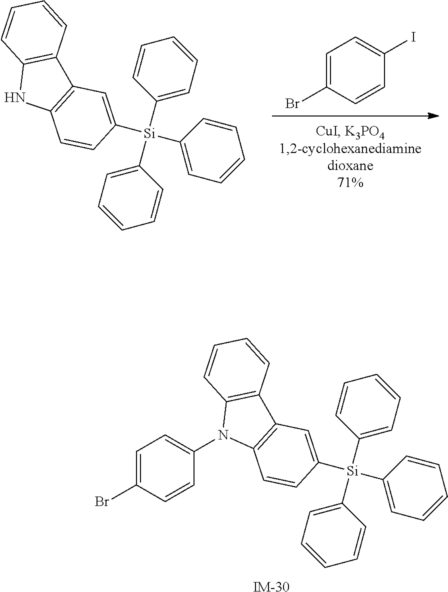

In an Ar atmosphere, in a 500 mL three-neck flask, 3-(triphenylsilyl)-9H-carbazole (20.00 g, 47.0 mmol), CuI (0.89 g, 0.1 equiv, 4.7 mmol), K₃PO₄ (29.92 g, 3.0 equiv, 140.9 mmol), 1-bromo-4-iodobenzene (66.47 g, 5.0 equiv, 235.0 mmol), 1,4-dioxane (235 mL), and 1,2-cyclohexanediamine (1.07 g, 0.2 equiv, 9.40 mmol) were sequentially added and heated and stirred under reflux. After the reaction solution was air-cooled to room temperature, the organic layer was fractionated by adding water to the reaction solvent and performing a suction filtration with celite. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-30 (19.37 g, yield 71%).

By measuring FAB-MS, a mass number of m/z=580 was observed by molecular ion peak, thereby identifying Intermediate IM-30.

Synthesis of Intermediate IM-31

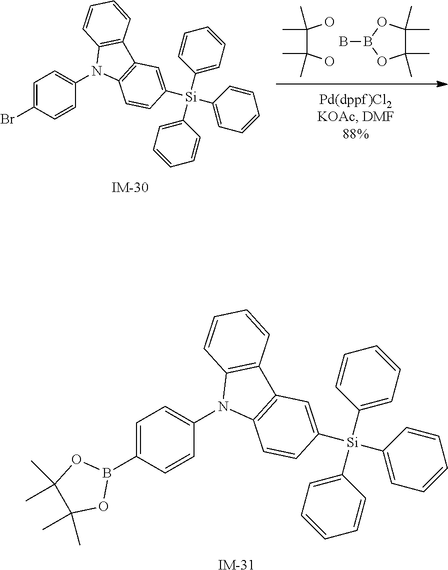

In an Ar atmosphere, in a 300 mL three-neck flask, Intermediate IM-30 (15.00 g, 25.8 mmol), Pd(dppf)Cl₂ (2.11 g, 0.1 equiv, 2.6 mmol), KOAc (5.07 g, 2.0 equiv, 51.7 mmol), DMF (130 mL) and bis(pinacolato)dibron (7.87 g, 1.2 equiv, 31.0 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-31 (14.27 g, yield 88%).

By measuring FAB-MS, a mass number of m/z=627 was observed by molecular ion peak, thereby identifying Intermediate IM-31.

Synthesis of Compound C30

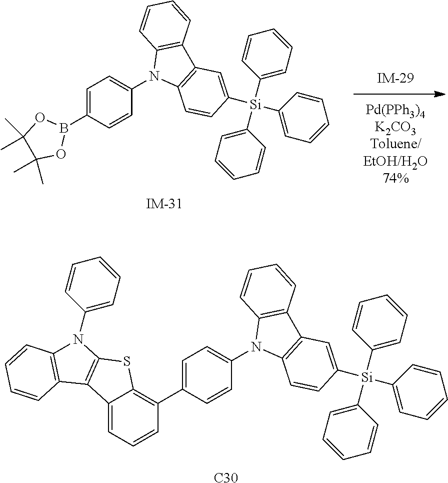

In an Ar atmosphere, in a 300 mL three-neck flask, Intermediate IM-29 (5.00 g, 13.2 mmol), Intermediate IM-31 (8.30 g, 1.0 equiv, 13.2 mmol), K₂CO₃ (5.48 g, 3.0 equiv, 39.7 mmol), Pd(PPh₃)₄ (0.76 g, 0.05 equiv, 0.7 mmol), and a mixed solution of toluene/EtOH/H₂O (4/2/1) (92 mL) were sequentially added and heated and stirred at about 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Compound C30 (7.82 g, yield 74%).

By measuring FAB-MS, a mass number of m/z=799 was observed by molecular ion peak, thereby identifying Compound C30.

(14) Synthesis of Compound C62

Synthesis of Intermediate IM-32

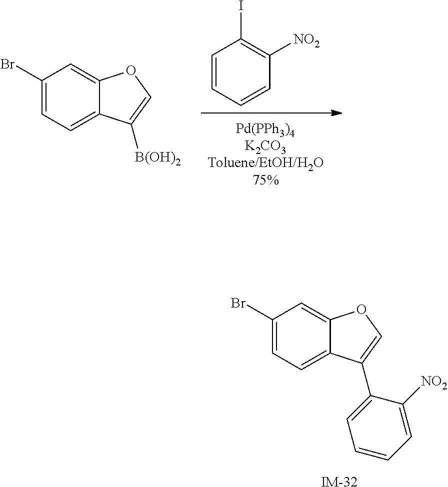

In an Ar atmosphere, in a 2000 mL three-neck flask, (6-bromobenzofuran-3-yl)boronic acid (35.00 g, 145.3 mmol), 1-iodo-2-nitrobenzene (39.80 g, 1.1 equiv, 159.9 mmol), K₂CO₃ (60.25 g, 3.0 equiv, 436.0 mmol), Pd(PPh₃)₄ (8.40 g, 0.05 equiv, 7.3 mmol), and a mixed solution of toluene/EtOH/H₂O (4/2/1) (1017 mL) were sequentially added and heated and stirred at about 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-32 (34.67 g, yield 75%).

By measuring FAB-MS, a mass number of m/z=318 was observed by molecular ion peak, thereby identifying Intermediate IM-32.

Synthesis of Intermediate IM-33

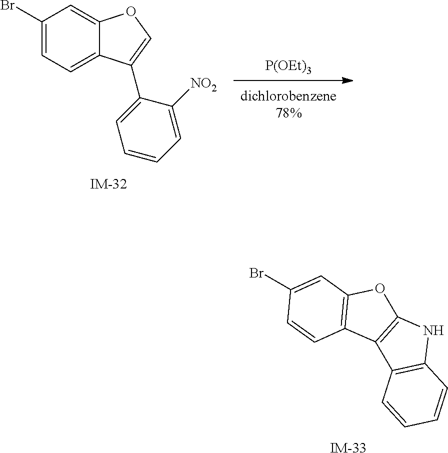

In an Ar atmosphere, in a 500 mL three-neck flask, Intermediate IM-32 (30.00 g, 94.3 mmol), o-dichlorobenzene (188 mL) and P(OEt)₃ (62.68 g, 4 equiv, 377.2 mmol) were sequentially added and heated and stirred at about 160° C. After the reaction solution was air-cooled to room temperature, the reaction solvent was removed by distillation, and then the resulting crude product was purified by silica gel column chromatography (using a mixed solvent of hexane and toluene as an eluent) to obtain Intermediate IM-33 (21.05 g, yield 78%).

By measuring FAB-MS, a mass number of m/z=286 was observed by molecular ion peak, thereby identifying Intermediate IM-33.

Synthesis of Intermediate IM-34

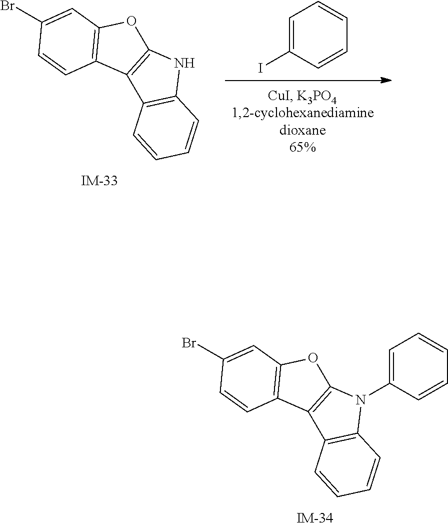

In an Ar atmosphere, in a 500 mL three-neck flask, Intermediate IM-33 (15.00 g, 52.4 mmol), CuI (1.00 g, 0.1 equiv, 5.2 mmol), K₃PO₄ (33.38 g, 3.0 equiv, 157.3 mmol), iodobenzene (21.39 g, 2.0 equiv, 104.8 mmol), 1,4-dioxane (262 mL), and 1,2-cyclohexanediamine (1.20 g, 0.2 equiv, 10.5 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-34 (12.34 g, yield 65%).

By measuring FAB-MS, a mass number of m/z=362 was observed by molecular ion peak, thereby identifying Intermediate IM-34.

Synthesis of Intermediate IM-35

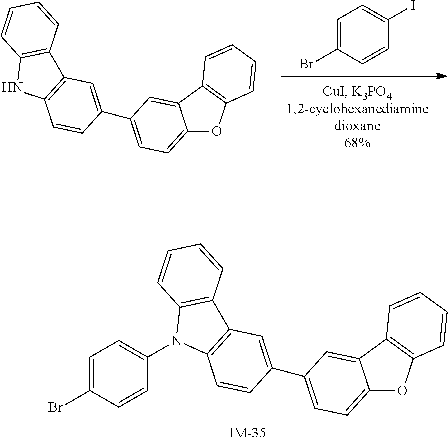

In an Ar atmosphere, in a 500 mL three-neck flask, 3-(dibenzofuran-2-yl)-9H-carbazole (20.00 g, 60.0 mmol), CuI (1.15 g, 0.1 equiv, 6.0 mmol), K₃PO₄ (38.20 g, 3.0 equiv, 180.0 mmol), 1-bromo-4-iodobenzene (84.86 g, 5.0 equiv, 299.9 mmol), 1,4-dioxane (300 mL), and 1,2-cyclohexanediamine (1.37 g, 0.2 equiv, 12.0 mmol) were sequentially added and heated and stirred under reflux. After the reaction solution was air-cooled to room temperature, the organic layer was fractionated by adding water to the reaction solvent and performing a suction filtration with celite. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-35 (19.92 g, yield 68%).

By measuring FAB-MS, a mass number of m/z=488 was observed by molecular ion peak, thereby identifying Intermediate IM-35.

Synthesis of Intermediate IM-36

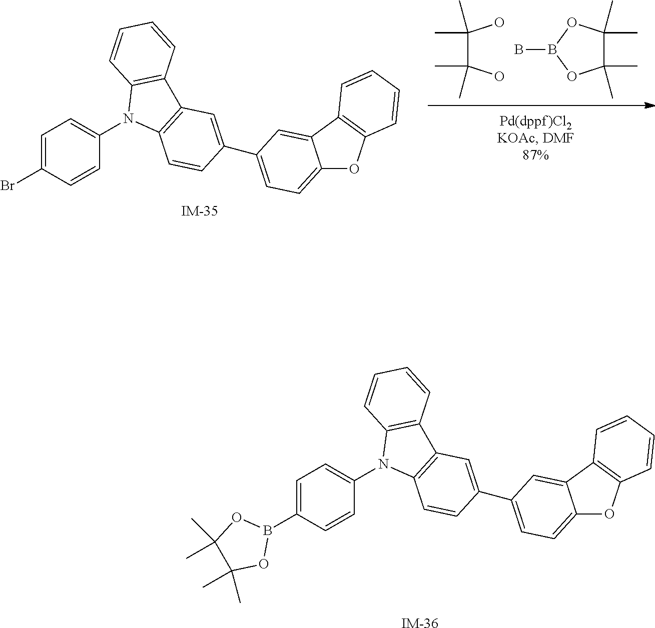

In an Ar atmosphere, in a 300 mL three-neck flask, Intermediate IM-35 (15.00 g, 30.7 mmol), Pd(dppf)Cl₂ (2.50 g, 0.1 equiv, 3.1 mmol), KOAc (6.03 g, 2.0 equiv, 61.4 mmol), DMF (154 mL) and bis(pinacolato)dibron (9.36 g, 1.2 equiv, 36.9 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-36 (14.31 g, yield 87%).

By measuring FAB-MS, a mass number of m/z=535 was observed by molecular ion peak, thereby identifying Intermediate IM-36.

Synthesis of Compound C62

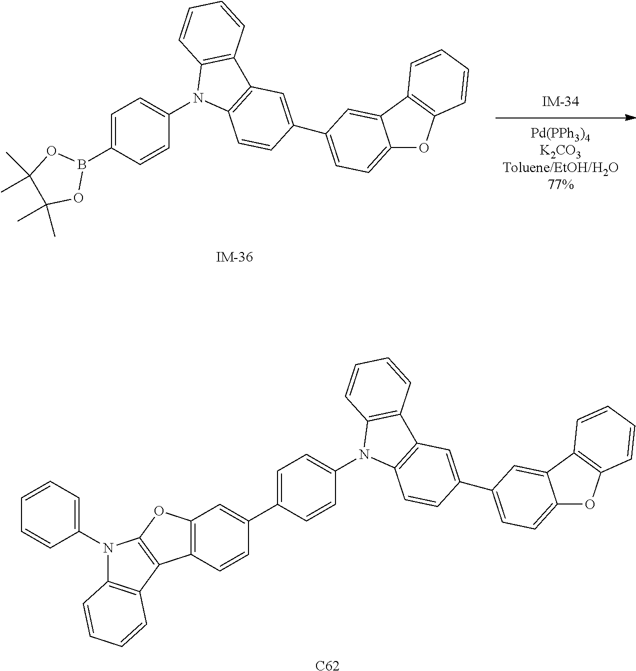

In an Ar atmosphere, in a 300 mL three-neck flask, Intermediate IM-34 (5.00 g, 13.8 mmol), Intermediate IM-36 (7.39 g, 1.0 equiv, 13.8 mmol), K₂CO₃ (5.72 g, 3.0 equiv, 41.4 mmol), Pd(PPh₃)₄ (0.80 g, 0.05 equiv, 0.7 mmol), and a mixed solution of toluene/EtOH/H₂O (4/2/1) (97 mL) were sequentially added and heated and stirred at about 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Compound C62 (7.34 g, yield 77%).

By measuring FAB-MS, a mass number of m/z=690 was observed by molecular ion peak, thereby identifying Compound C62.

(15) Synthesis of Compound C114

Synthesis of Intermediate IM-37

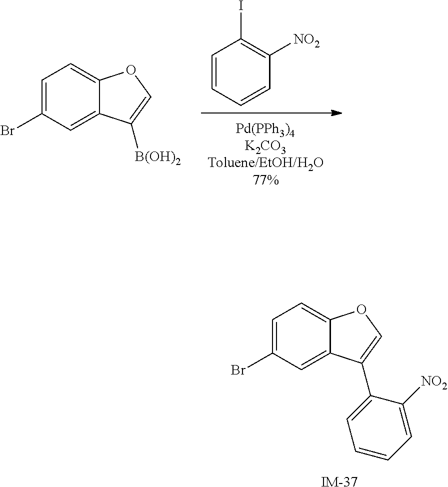

In an Ar atmosphere, in a 2000 mL three-neck flask, (5-bromobenzofuran-3-yl)boronic acid (35.00 g, 145.3 mmol), 1-iodo-2-nitrobenzene (39.80 g, 1.1 equiv, 159.9 mmol), K₂CO₃ (60.25 g, 3.0 equiv, 436.0 mmol), Pd(PPh₃)₄ (8.40 g, 0.05 equiv, 7.3 mmol), and a mixed solution of toluene/EtOH/H₂O (4/2/1) (1017 mL) were sequentially added and heated and stirred at about 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-37 (35.60 g, yield 77%).

By measuring FAB-MS, a mass number of m/z=318 was observed by molecular ion peak, thereby identifying Intermediate IM-37.

Synthesis of Intermediate IM-38

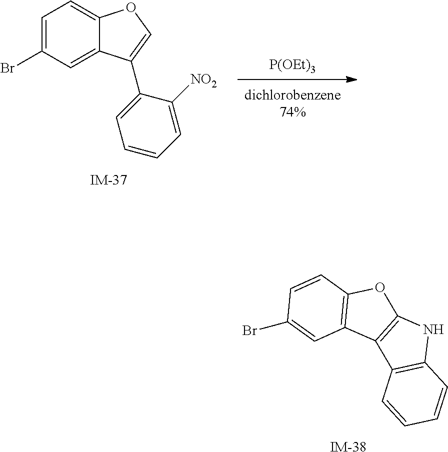

In an Ar atmosphere, in a 500 mL three-neck flask, Intermediate IM-37 (30.00 g, 94.3 mmol), o-dichlorobenzene (188 mL) and P(OEt)₃ (62.68 g, 4 equiv, 377.2 mmol) were sequentially added and heated and stirred at about 160° C. After the reaction solution was air-cooled to room temperature, the reaction solvent was removed by distillation, and then the resulting crude product was purified by silica gel column chromatography (using a mixed solvent of hexane and toluene as an eluent) to obtain Intermediate IM-38 (19.97 g, yield 74%).

By measuring FAB-MS, a mass number of m/z=286 was observed by molecular ion peak, thereby identifying Intermediate IM-38.

Synthesis of Intermediate IM-39

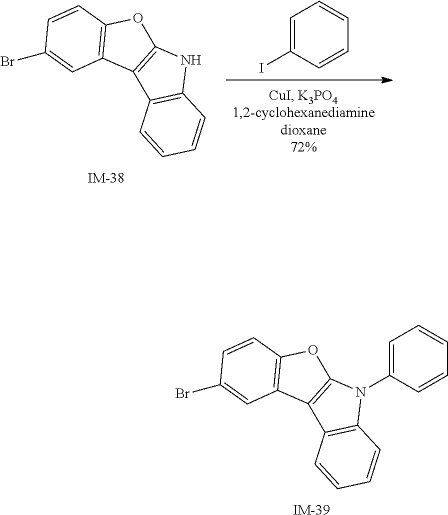

In an Ar atmosphere, in a 500 mL three-neck flask Intermediate IM-38 (15.00 g, 52.4 mmol), CuI (1.00 g, 0.1 equiv, 5.2 mmol), K₃PO₄ (33.38 g, 3.0 equiv, 157.3 mmol), iodobenzene (21.39 g, 2.0 equiv, 104.8 mmol), 1,4-dioxane (262 mL), and 1,2-cyclohexanediamine (1.20 g, 0.2 equiv, 10.5 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-39 (13.67 g, yield 72%).

By measuring FAB-MS, a mass number of m/z=362 was observed by molecular ion peak, thereby identifying Intermediate IM-39.

Synthesis of Intermediate IM-40

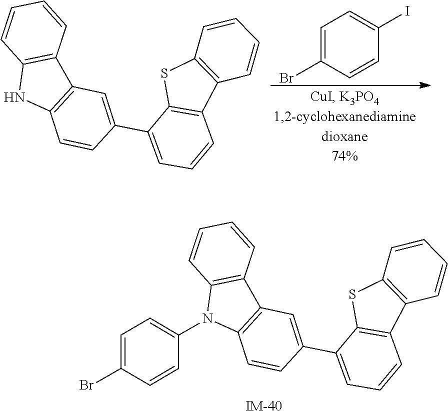

In an Ar atmosphere, in a 500 mL three-neck flask, 3-(dibenzothiophen-4-yl)-9H-carbazole (20.00 g, 57.2 mmol), CuI (1.09 g, 0.1 equiv, 5.72 mmol), K₃PO₄ (36.45 g, 3.0 equiv, 171.7 mmol), 1-bromo-4-iodobenzene (80.96 g, 5.0 equiv, 286.2 mmol), 1,4-dioxane (286 mL), and 1,2-cyclohexanediamine (1.31 g, 0.2 equiv, 11.4 mmol) were sequentially added and heated and stirred under reflux. After the reaction solution was air-cooled to room temperature, the organic layer was fractionated by adding water to the reaction solvent and performing a suction filtration with celite. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-40 (21.36 g, yield 74%).

By measuring FAB-MS, a mass number of m/z=504 was observed by molecular ion peak, thereby identifying Intermediate IM-40.

Synthesis of Intermediate IM-41

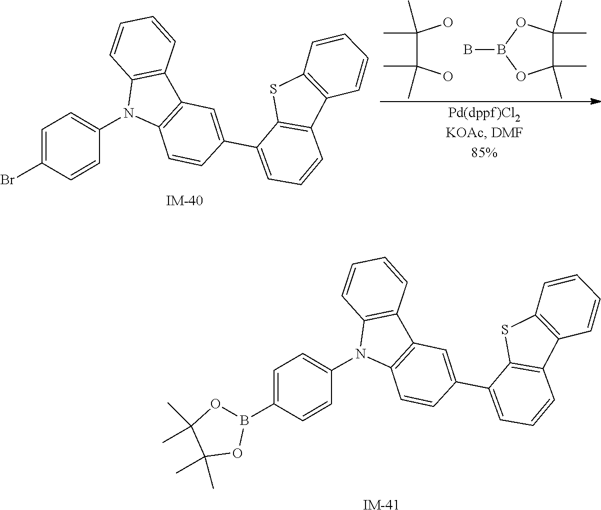

In an Ar atmosphere, in a 300 mL three-neck flask, Intermediate IM-40 (15.00 g, 29.7 mmol), Pd(dppf)Cl₂ (2.43 g, 0.1 equiv, 3.0 mmol), KOAc (5.84 g, 2.0 equiv, 59.5 mmol), DMF (148 mL) and bis(pinacolato)dibron (9.06 g, 1.2 equiv, 35.7 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-41 (13.94 g, yield 85%).

By measuring FAB-MS, a mass number of m/z=551 was observed by molecular ion peak, thereby identifying Intermediate IM-41.

Synthesis of Compound C114

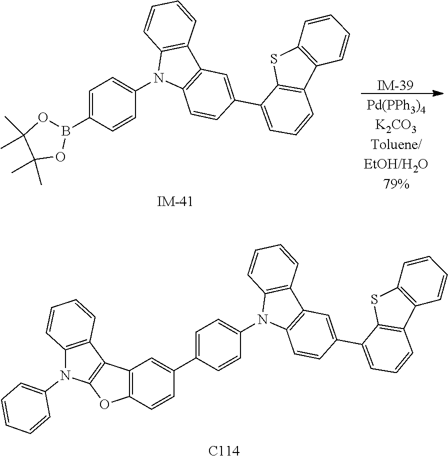

In an Ar atmosphere, in a 300 mL three-neck flask, Intermediate IM-39 (5.00 g, 13.8 mmol), Intermediate IM-41 (7.61 g, 1.0 equiv, 13.8 mmol), K₂CO₃ (5.72 g, 3.0 equiv, 41.4 mmol), Pd(PPh₃)₄ (0.80 g, 0.05 equiv, 0.7 mmol), and a mixed solution of toluene/EtOH/H₂O (4/2/1) (97 mL) were sequentially added and heated and stirred at about 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Compound C114 (7.71 g, yield 79%).

By measuring FAB-MS, a mass number of m/z=706 was observed by molecular ion peak, thereby identifying Compound C114.

(16) Synthesis of Compound C148

Synthesis of Intermediate IM-42

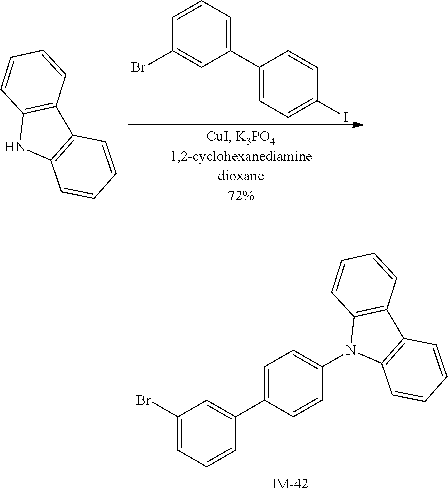

In an Ar atmosphere, in a 1000 mL three-neck flask, 9H-carbazol (20.00 g, 119.6 mmol), CuI (2.28 g, 0.1 equiv, 11.96 mmol), K₃PO₄ (76.17 g, 3.0 equiv, 358.8 mmol), 3-bromo-4′-iodo-1,1′-biphenyl (214.70 g, 5.0 equiv, 598.1 mmol), 1,4-dioxane (598 mL), and 1,2-cyclohexanediamine (2.73 g, 0.2 equiv, 23.9 mmol) were sequentially added and heated and stirred under reflux. After the reaction solution was air-cooled to room temperature, the organic layer was fractionated by adding water to the reaction solvent and performing a suction filtration with diatomaceous earth (sold under the trade designation CELITE by Imerys Minerals California, Inc. of San Jose, Calif. (hereinafter “celite”). The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-42 (34.30 g, yield 72%).

By measuring FAB-MS, a mass number of m/z=398 was observed by molecular ion peak, thereby identifying Intermediate IM-42.

Synthesis of Intermediate IM-43

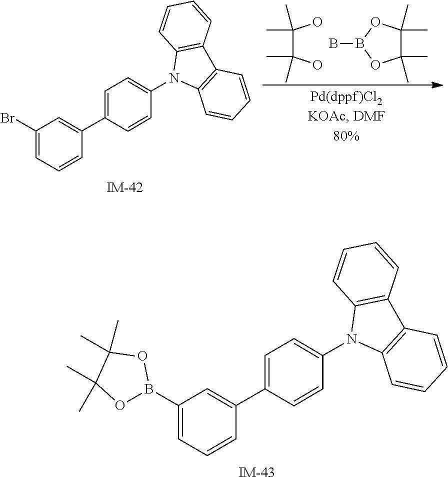

In an Ar atmosphere, in a 500 mL three-neck flask, Intermediate IM-42 (15.00 g, 37.7 mmol), Pd(dppf)Cl₂ (3.08 g, 0.1 equiv, 3.8 mmol), KOAc (7.39 g, 2.0 equiv, 75.3 mmol), DMF (188 mL) and bis(pinacolato)dibron (11.48 g, 1.2 equiv, 45.2 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-43 (13.42 g, yield 80%).

By measuring FAB-MS, a mass number of m/z=445 was observed by molecular ion peak, thereby identifying Intermediate IM-43.

Synthesis of Compound C148

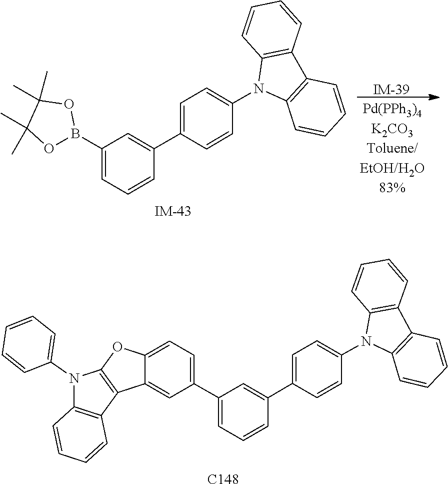

In an Ar atmosphere, in a 300 mL three-neck flask, Intermediate IM-39 (5.00 g, 13.8 mmol), Intermediate IM-43 (6.59 g, 1.0 equiv, 13.8 mmol), K₂CO₃ (5.72 g, 3.0 equiv, 41.4 mmol), Pd(PPh₃)₄ (0.80 g, 0.05 equiv, 0.7 mmol), and a mixed solution of toluene/EtOH/H₂O (4/2/1) (97 mL) were sequentially added and heated and stirred at about 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Compound C148 (6.88 g, yield 83%).

By measuring FAB-MS, a mass number of m/z=600 was observed by molecular ion peak, thereby identifying Compound C148.

(17) Synthesis of Compound C190

Synthesis of Intermediate IM-44

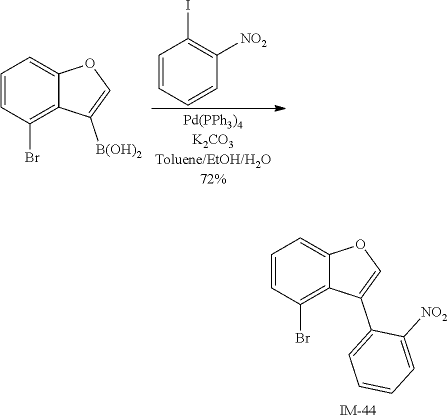

In an Ar atmosphere, in a 2000 mL three-neck flask, (4-bromobenzofuran-3-yl)boronic acid (35.00 g, 145.3 mmol), 1-iodo-2-nitrobenzene (39.80 g, 1.1 equiv, 159.9 mmol), K₂CO₃ (60.25 g, 3.0 equiv, 436.0 mmol), Pd(PPh₃)₄ (8.40 g, 0.05 equiv, 7.3 mmol), and a mixed solution of toluene/EtOH/H₂O (4/2/1) (1017 mL) were sequentially added and heated and stirred at about 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-44 (33.29 g, yield 72%).

By measuring FAB-MS, a mass number of m/z=318 was observed by molecular ion peak, thereby identifying Intermediate IM-44.

Synthesis of Intermediate IM-45

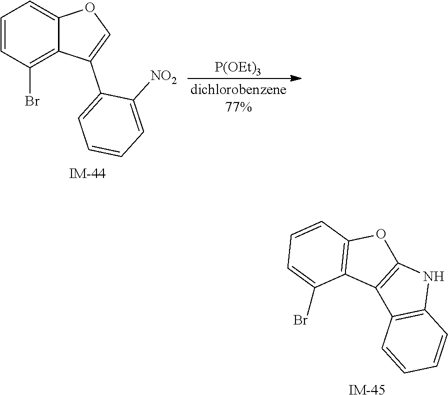

In an Ar atmosphere, in a 500 mL three-neck flask, Intermediate IM-44 (30.00 g, 94.3 mmol), o-dichlorobenzene (188 mL) and P(OEt)₃ (62.68 g, 4 equiv, 377.2 mmol) were sequentially added and heated and stirred at about 160° C. After the reaction solution was air-cooled to room temperature, the reaction solvent was removed by distillation, and then the resulting crude product was purified by silica gel column chromatography (using a mixed solvent of hexane and toluene as an eluent) to obtain Intermediate IM-45 (20.78 g, yield 77%).

By measuring FAB-MS, a mass number of m/z=286 was observed by molecular ion peak, thereby identifying Intermediate IM-45.

Synthesis of Intermediate IM-46

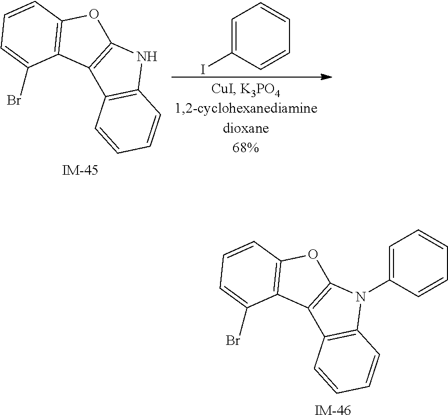

In an Ar atmosphere, in a 500 mL three-neck flask, Intermediate IM-45 (15.00 g, 52.4 mmol), CuI (1.00 g, 0.1 equiv, 5.2 mmol), K₃PO₄ (33.38 g, 3.0 equiv, 157.3 mmol), iodobenzene (21.39 g, 2.0 equiv, 104.8 mmol), 1,4-dioxane (262 mL), and 1,2-cyclohexanediamine (1.20 g, 0.2 equiv, 10.5 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-46 (12.91 g, yield 68%).

By measuring FAB-MS, a mass number of m/z=362 was observed by molecular ion peak, thereby identifying Intermediate IM-46.

Synthesis of Intermediate IM-47

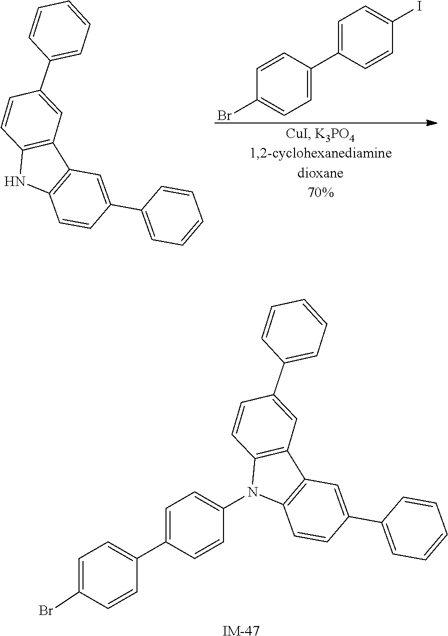

In an Ar atmosphere, in a 500 mL three-neck flask, 3,6-diphenyl-9H-carbazole (20.00 g, 62.6 mmol), CuI (1.19 g, 0.1 equiv, 6.3 mmol), K₃PO₄ (39.87 g, 3.0 equiv, 187.8 mmol), 3-bromo-4′-iodo-1,1′-biphenyl (112.39 g, 5.0 equiv, 313.1 mmol), 1,4-dioxane (313 mL), and 1,2-cyclohexanediamine (1.43 g, 0.2 equiv, 12.5 mmol) were sequentially added and heated and stirred under reflux. After the reaction solution was air-cooled to room temperature, the organic layer was fractionated by adding water to the reaction solvent and performing a suction filtration with celite. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-47 (24.83 g, yield 70%).

By measuring FAB-MS, a mass number of m/z=566 was observed by molecular ion peak, thereby identifying Intermediate IM-47.

Synthesis of Intermediate IM-48

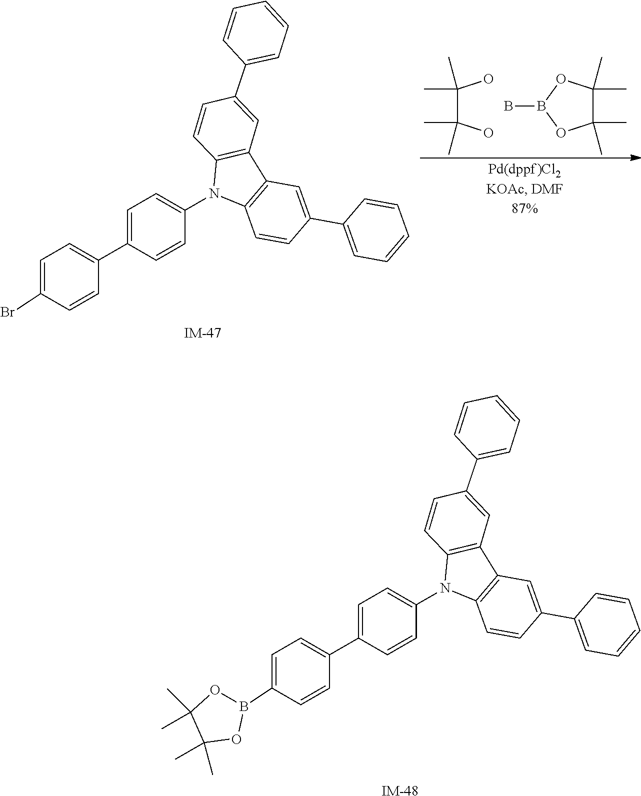

In an Ar atmosphere, in a 300 mL three-neck flask, Intermediate IM-47 (15.00 g, 26.5 mmol), Pd(dppf)Cl₂ (2.16 g, 0.1 equiv, 2.6 mmol), KOAc (5.20 g, 2.0 equiv, 53.0 mmol), DMF (132 mL) and bis(pinacolato)dibron (8.07 g, 1.2 equiv, 31.8 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Intermediate IM-48 (14.50 g, yield 87%).

By measuring FAB-MS, a mass number of m/z=629 was observed by molecular ion peak, thereby identifying Intermediate IM-48.

Synthesis of Compound C190

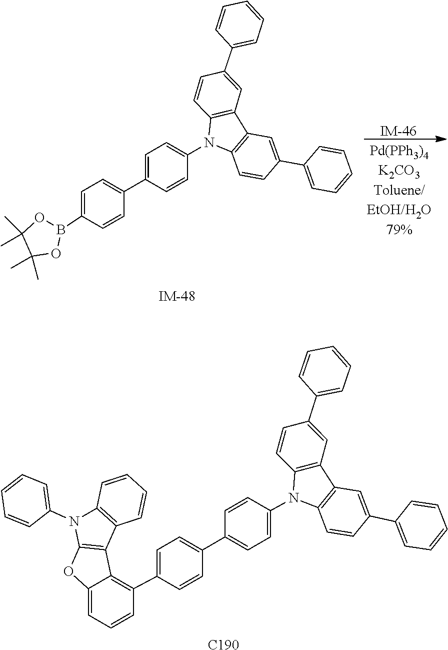

In an Ar atmosphere, in a 300 mL three-neck flask, Intermediate IM-46 (5.00 g, 13.8 mmol), Intermediate IM-48 (8.69 g, 1.0 equiv, 13.8 mmol), K₂CO₃ (5.72 g, 3.0 equiv, 41.4 mmol), Pd(PPh₃)₄ (0.80 g, 0.05 equiv, 0.7 mmol), and a mixed solution of toluene/EtOH/H₂O (4/2/1) (97 mL) were sequentially added and heated and stirred at about 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Compound C190 (8.21 g, yield 79%).

By measuring FAB-MS, a mass number of m/z=752 was observed by molecular ion peak, thereby identifying Compound C190.

(18) Synthesis of Compound A59

Synthesis of Compound A59

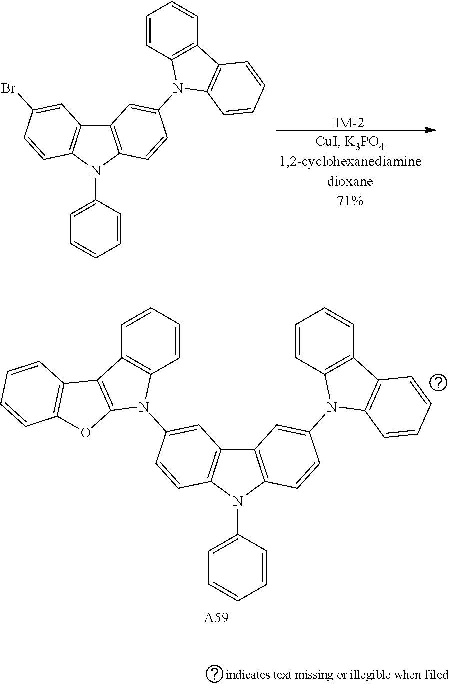

In an Ar atmosphere, in a 300 mL three-neck flask, Intermediate IM-2 (5.00 g, 24.1 mmol), CuI (0.46 g, 0.1 equiv, 2.4 mmol), K₃PO₄ (15.36 g, 3.0 equiv, 72.4 mmol), 6-bromo-9-phenyl-9H-3,9′-bicarbazole (11.76 g, 1.0 equiv, 24.1 mmol), 1,4-dioxane (120 mL), and 1,2-cyclohexanediamine (0.55 g, 0.2 equiv, 4.8 mmol) were sequentially added and heated and stirred under reflux. After the reaction solution was air-cooled to room temperature, the organic layer was fractionated by adding water to the reaction solvent. The organic layer was further extracted by adding toluene to a water layer, and then the combined organic layers were washed with saline and dried over MgSO₄. The compound MgSO₄ was filtered off and the organic layers were concentrated, and then the resulting crude product was purified by silica gel column chromatography (using a mixed solvent of hexane and toluene as an eluent) to obtain Compound A59 which is solid (10.51 g, yield 71%).

By measuring FAB-MS, a mass number of m/z=613 was observed by molecular ion peak, thereby identifying Compound A59.

(19) Synthesis of Compound C222

Synthesis of Compound C222

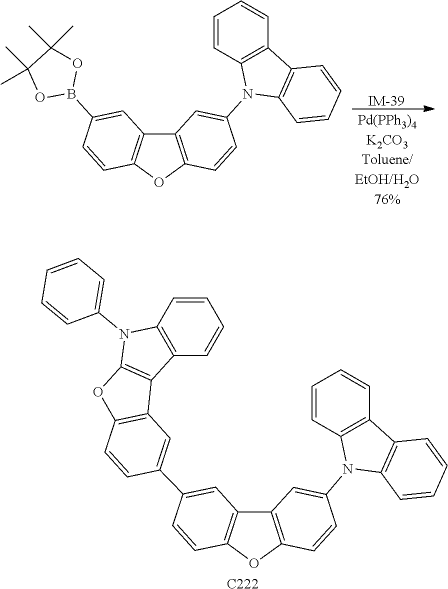

In an Ar atmosphere, in a 300 mL three-neck flask, Intermediate IM-39 (5.00 g, 13.8 mmol), 9-[8-(4,4,5,5-tetramethyl-1,3,2-dioxaborolan-2-yl)dibenzofuran-2-yl]-9H-carbazole (6.34 g, 1.0 equiv, 13.8 mmol), K₂CO₃ (5.72 g, 3.0 equiv, 41.4 mmol), Pd(PPh₃)₄ (0.80 g, 0.05 equiv, 0.7 mmol), and a mixed solution of toluene/EtOH/H₂O (4/2/1, 97 mL) were sequentially added, and then heated and stirred at 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Compound C222 (6.45 g, yield 76%).

By measuring FAB-MS, a mass number of m/z=614 was observed by molecular ion peak, thereby identifying Compound C222

(20) Synthesis of Compound C259

Synthesis of Compound C259

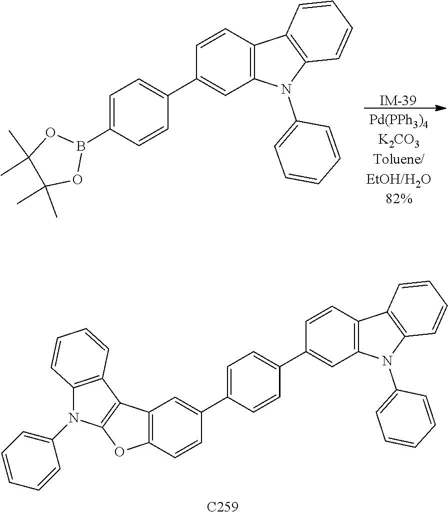

In an Ar atmosphere, in a 300 mL three-neck flask, Intermediate IM-39 (5.00 g, 13.8 mmol), 9-phenyl-2-[4-(4,4,5,5-tetramethyl-1,3,2-dioxaborolan-2-yl)phenyl]-9H-carbazole 6.15 g (1.0 equiv, 13.8 mmol), K₂CO₃ (5.72 g, 3.0 equiv, 41.4 mmol), Pd(PPh₃)₄ (0.80 g, 0.05 equiv, 0.7 mmol), and a mixed solution of toluene/EtOH/H₂O (4/2/1, 97 mL) were sequentially added, and then heated and stirred at 80° C. After air-cooled to room temperature, the reaction solution was extracted with toluene. A water layer was removed, and the organic layers were washed with saturated saline and then dried over MgSO₄. The compound MgSO₄ was filtered off and the organic layers were concentrated, and then the resulting crude product was purified by silica gel column chromatography (using a mixed solvent of hexane and toluene as an eluent) to obtain Compound C259 (6.80 g, yield 82%).

By measuring FAB-MS, a mass number of m/z=600 was observed by molecular ion peak, thereby identifying Compound C259.

2. Manufacture and Evaluation of Light Emitting Device Including Nitrogen-Containing Compound

(1) Manufacture and Evaluation of Light Emitting Device in which Nitrogen-Containing Compound is Applied as Hole Transport Layer Material and Fluorescence Emission Compound is used in Emission Layer

Manufacture of Light Emitting Device

Compounds A3, A36, A45, A50, A54, A74, B4, B99, B107, B138, B151, B263, C30, C62, C114, C148, C190, A59, C222, and C259 as described above were used as a hole transport layer material to manufacture the light emitting devices of Examples 1-1 to 1-20, respectively.

### EXAMPLE COMPOUNDS

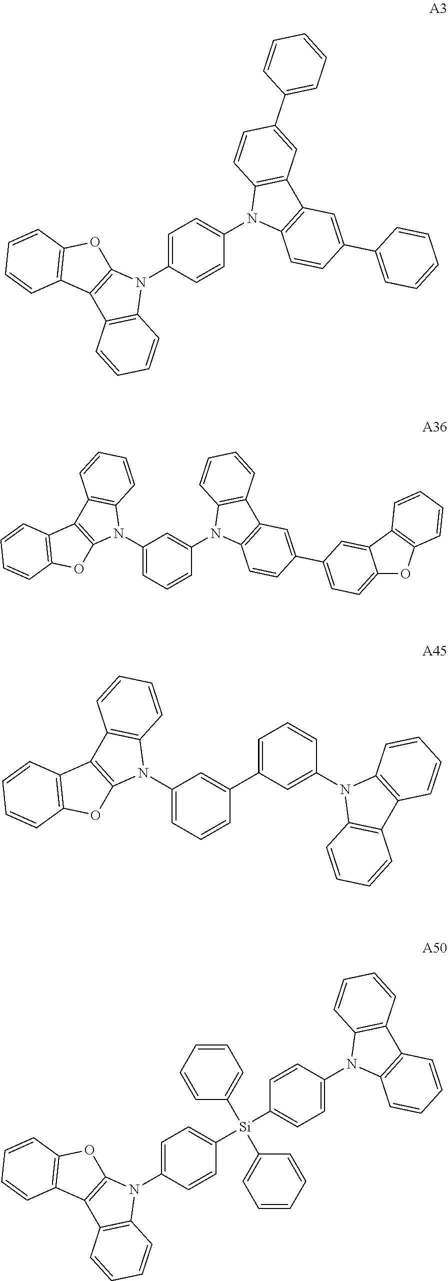 [SMILES: c1ccc(-c2ccc3c(c2)c2cc(-c4ccccc4)ccc2n3-c2ccc(-n3c4ccccc4c4c5ccccc5oc43)cc2)cc1 ; c1cc(-n2c3ccccc3c3cc(-c4ccc5oc6ccccc6c5c4)ccc32)cc(-n2c3ccccc3c3c4ccccc4oc32)c1 ; c1cc(-c2cccc(-n3c4ccccc4c4c5ccccc5oc43)c2)cc(-n2c3ccccc3c3ccccc32)c1 ; c1ccc([Si](c2ccccc2)(c2ccc(-n3c4ccccc4c4ccccc43)cc2)c2ccc(-n3c4ccccc4c4c5ccccc5oc43)cc2)cc1]

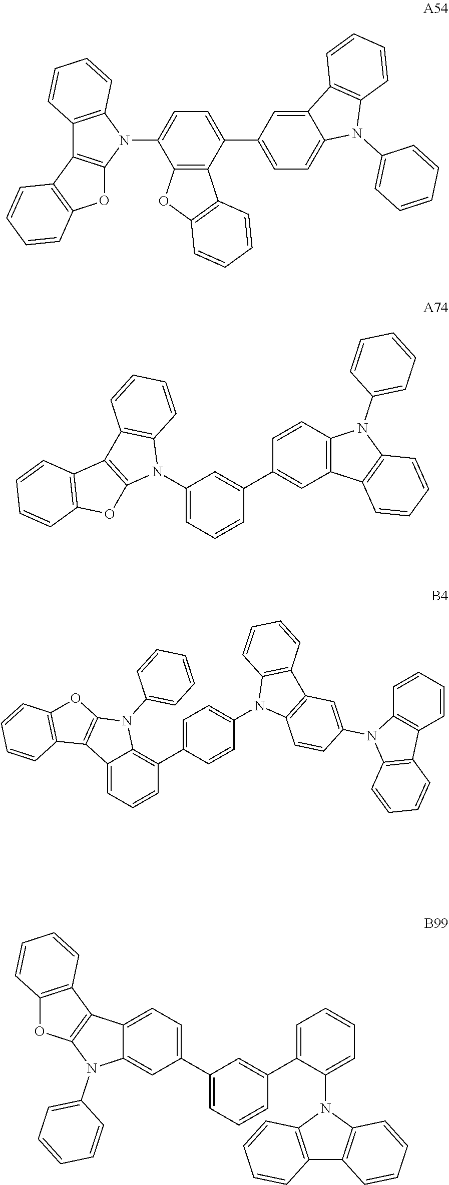 [SMILES: c1ccc(-n2c3ccccc3c3cc(-c4ccc(-n5c6ccccc6c6c7ccccc7oc65)c5oc6ccccc6c45)ccc32)cc1 ; c1ccc(-n2c3ccccc3c3cc(-c4cccc(-n5c6ccccc6c6c7ccccc7oc65)c4)ccc32)cc1 ; c1ccc(-n2c3oc4ccccc4c3c3cccc(-c4ccc(-n5c6ccccc6c6cc(-n7c8ccccc8c8ccccc87)ccc65)cc4)c32)cc1 ; c1ccc(-n2c3cc(-c4cccc(-c5ccccc5-n5c6ccccc6c6ccccc65)c4)ccc3c3c4ccccc4oc32)cc1]

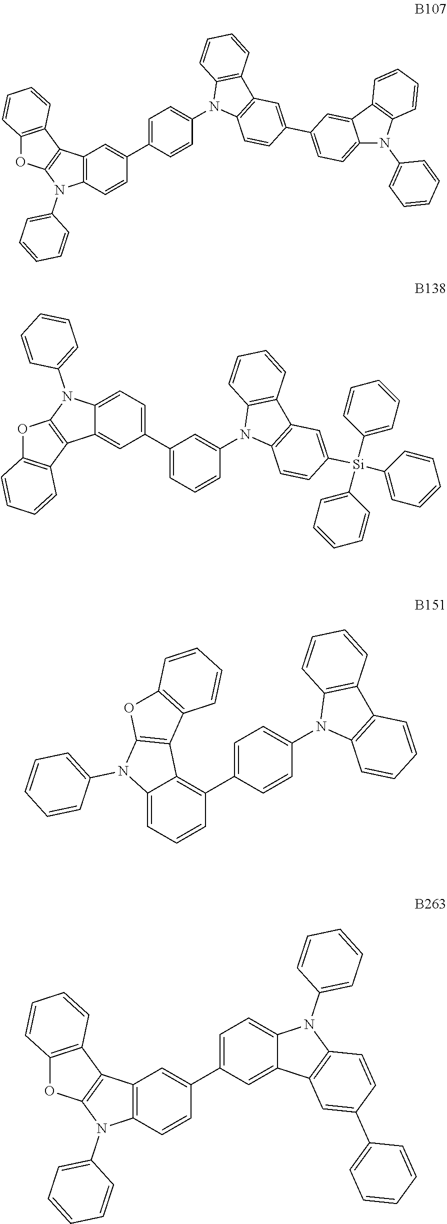 [SMILES: c1ccc(-n2c3ccccc3c3cc(-c4ccc5c(c4)c4ccccc4n5-c4ccc(-c5ccc6c(c5)c5c7ccccc7oc5n6-c5ccccc5)cc4)ccc32)cc1 ; c1ccc(-n2c3ccc(-c4cccc(-n5c6ccccc6c6cc([Si](c7ccccc7)(c7ccccc7)c7ccccc7)ccc65)c4)cc3c3c4ccccc4oc32)cc1 ; c1ccc(-n2c3cccc(-c4ccc(-n5c6ccccc6c6ccccc65)cc4)c3c3c4ccccc4oc32)cc1 ; c1ccc(-c2ccc3c(c2)c2cc(-c4ccc5c(c4)c4c6ccccc6oc4n5-c4ccccc4)ccc2n3-c2ccccc2)cc1]

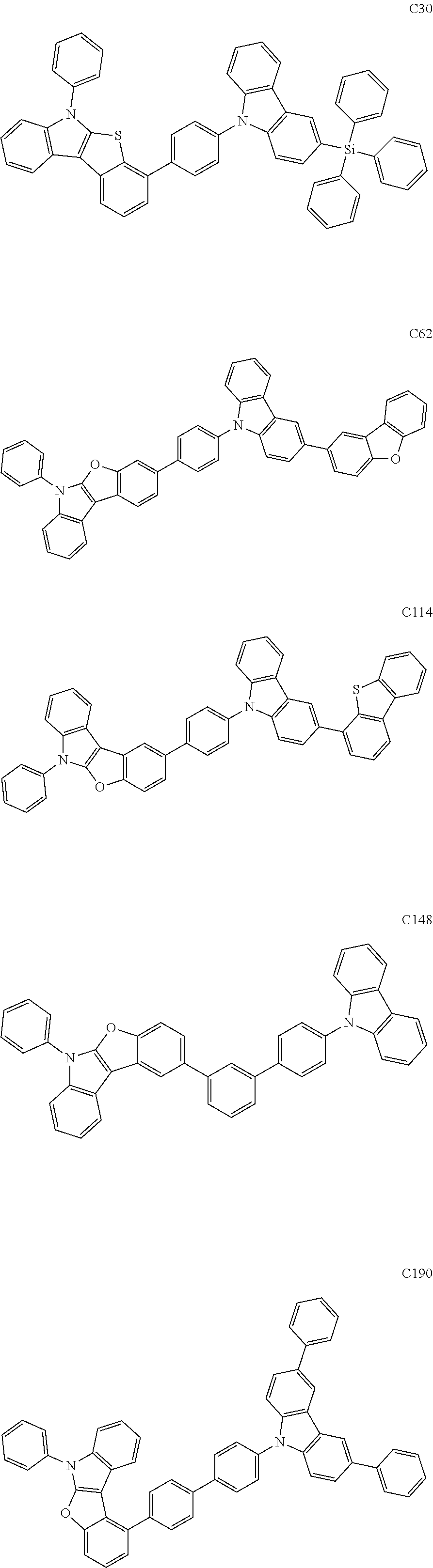 [SMILES: c1ccc(-n2c3ccccc3c3c4cccc(-c5ccc(-n6c7ccccc7c7cc([Si](c8ccccc8)(c8ccccc8)c8ccccc8)ccc76)cc5)c4sc32)cc1 ; c1ccc(-n2c3ccccc3c3c4ccc(-c5ccc(-n6c7ccccc7c7cc(-c8ccc9oc%10ccccc%10c9c8)ccc76)cc5)cc4oc32)cc1 ; c1ccc(-n2c3ccccc3c3c4cc(-c5ccc(-n6c7ccccc7c7cc(-c8cccc9c8sc8ccccc89)ccc76)cc5)ccc4oc32)cc1 ; c1ccc(-n2c3ccccc3c3c4cc(-c5cccc(-c6ccc(-n7c8ccccc8c8ccccc87)cc6)c5)ccc4oc32)cc1 ; c1ccc(-c2ccc3c(c2)c2cc(-c4ccccc4)ccc2n3-c2ccc(-c3ccc(-c4cccc5oc6c(c7ccccc7n6-c6ccccc6)c45)cc3)cc2)cc1]

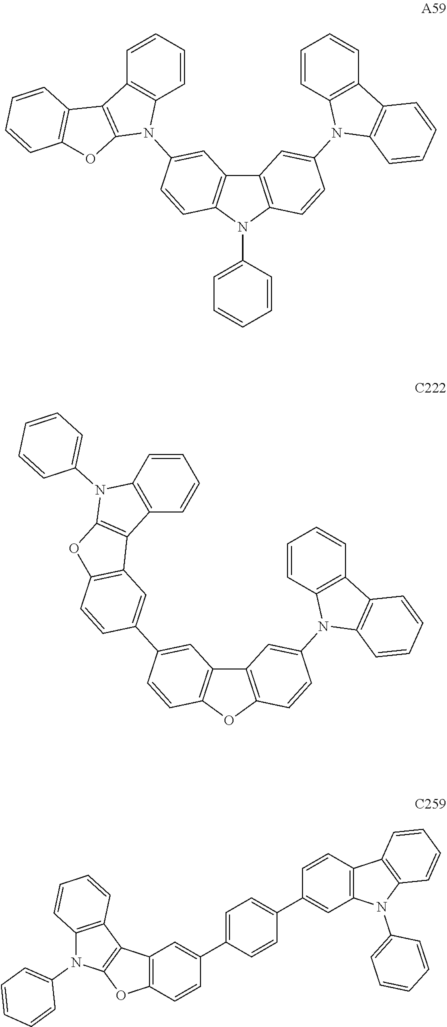 [SMILES: c1ccc(-n2c3ccc(-n4c5ccccc5c5ccccc54)cc3c3cc(-n4c5ccccc5c5c6ccccc6oc54)ccc32)cc1 ; c1ccc(-n2c3ccccc3c3c4cc(-c5ccc6oc7ccc(-n8c9ccccc9c9ccccc98)cc7c6c5)ccc4oc32)cc1 ; c1ccc(-n2c3ccccc3c3ccc(-c4ccc(-c5ccc6oc7c(c6c5)c5ccccc5n7-c5ccccc5)cc4)cc32)cc1]

Comparative Example Compounds R1 to R17 below were used to manufacture devices of Comparative Examples.

Comparative Example Compounds

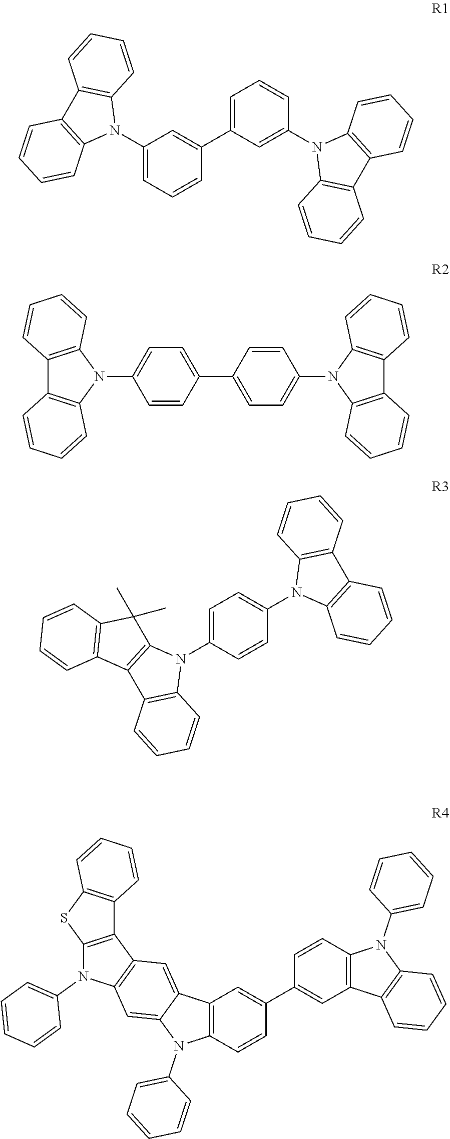 [SMILES: c1cc(-c2cccc(-n3c4ccccc4c4ccccc43)c2)cc(-n2c3ccccc3c3ccccc32)c1 ; c1ccc2c(c1)c1ccccc1n2-c1ccc(-c2ccc(-n3c4ccccc4c4ccccc43)cc2)cc1 ; CC1(C)c2ccccc2-c2c1n(-c1ccc(-n3c4ccccc4c4ccccc43)cc1)c1ccccc21 ; c1ccc(-n2c3ccccc3c3cc(-c4ccc5c(c4)c4cc6c7c8ccccc8sc7n(-c7ccccc7)c6cc4n5-c4ccccc4)ccc32)cc1]

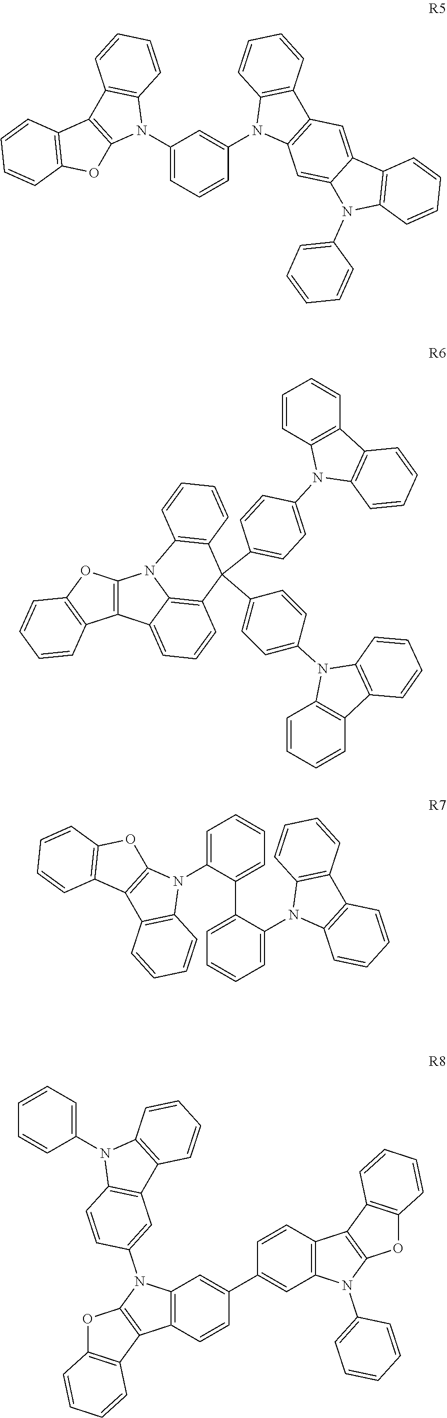 [SMILES: c1ccc(-n2c3ccccc3c3cc4c5ccccc5n(-c5cccc(-n6c7ccccc7c7c8ccccc8oc76)c5)c4cc32)cc1 ; c1ccc2c(c1)-n1c3oc4ccccc4c3c3cccc(c31)C2(c1ccc(-n2c3ccccc3c3ccccc32)cc1)c1ccc(-n2c3ccccc3c3ccccc32)cc1 ; c1ccc(-n2c3ccccc3c3ccccc32)c(-c2ccccc2-n2c3ccccc3c3c4ccccc4oc32)c1 ; c1ccc(-n2c3ccccc3c3cc(-n4c5cc(-c6ccc7c8c9ccccc9oc8n(-c8ccccc8)c7c6)ccc5c5c6ccccc6oc54)ccc32)cc1]

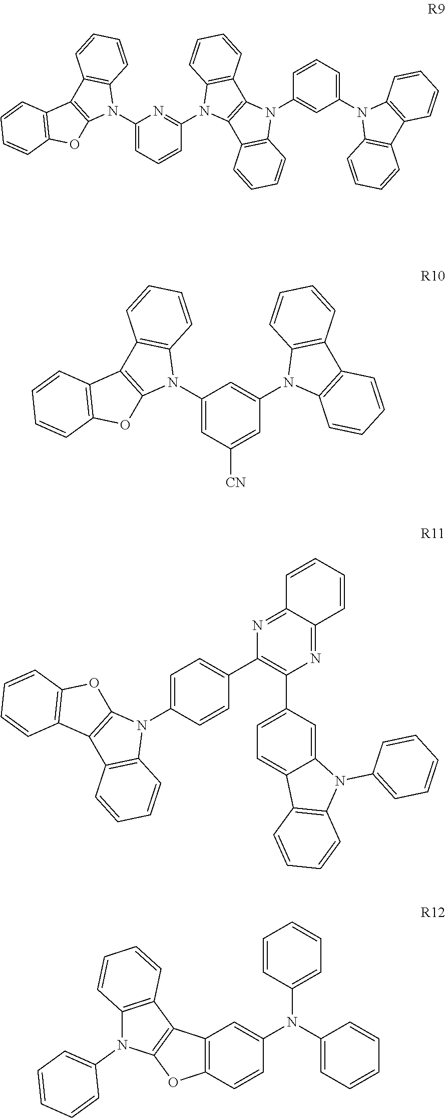 [SMILES: c1cc(-n2c3ccccc3c3ccccc32)cc(-n2c3ccccc3c3c2c2ccccc2n3-c2cccc(-n3c4ccccc4c4c5ccccc5oc43)n2)c1 ; N#Cc1cc(-n2c3ccccc3c3ccccc32)cc(-n2c3ccccc3c3c4ccccc4oc32)c1 ; c1ccc(-n2c3ccccc3c3ccc(-c4nc5ccccc5nc4-c4ccc(-n5c6ccccc6c6c7ccccc7oc65)cc4)cc32)cc1 ; c1ccc(N(c2ccccc2)c2ccc3oc4c(c3c2)c2ccccc2n4-c2ccccc2)cc1]

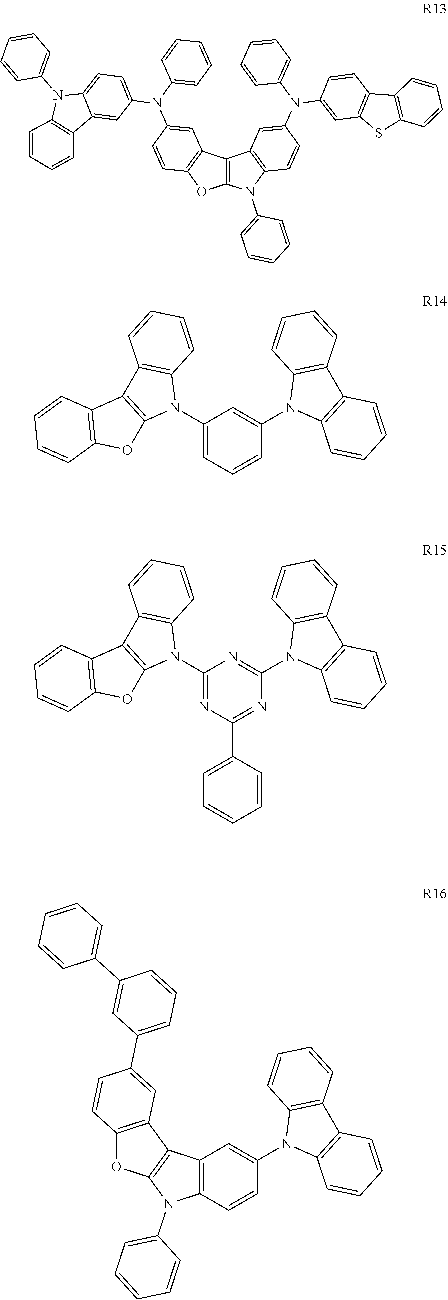 [SMILES: c1ccc(N(c2ccc3oc4c(c3c2)c2cc(N(c3ccccc3)c3ccc5c(c3)sc3ccccc35)ccc2n4-c2ccccc2)c2ccc3c(c2)c2ccccc2n3-c2ccccc2)cc1 ; c1cc(-n2c3ccccc3c3ccccc32)cc(-n2c3ccccc3c3c4ccccc4oc32)c1 ; c1ccc(-c2nc(-n3c4ccccc4c4ccccc43)nc(-n3c4ccccc4c4c5ccccc5oc43)n2)cc1 ; c1ccc(-c2cccc(-c3ccc4oc5c(c4c3)c3cc(-n4c6ccccc6c6ccccc64)ccc3n5-c3ccccc3)c2)cc1]

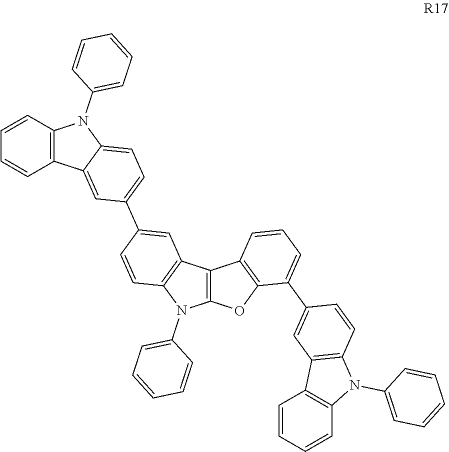 [SMILES: c1ccc(-n2c3ccccc3c3cc(-c4ccc5c(c4)c4c6cccc(-c7ccc8c(c7)c7ccccc7n8-c7ccccc7)c6oc4n5-c4ccccc4)ccc32)cc1]

The light emitting device of an example including the nitrogen-containing compound of an example in a hole transport layer was manufactured as follows. Examples 1-1 to 1-20 correspond to the light emitting devices manufactured by using Compounds A3, A36, A45, A50, A54, A74, B4, B99, B107, B138, B151, B263, C30, C62, C114, C148, C190, A59, C222, and C259 which are Example Compounds as described above as a hole transport layer material, respectively. Comparative Examples 1-1 to 1-17 correspond to the light emitting devices manufactured by using Comparative Example Compounds R1 to R17 as a hole transport layer material, respectively.

The compound ITO was used to form a 150 nm-thick first electrode, 4,4′,4″-tris[N(2-naphthyl)-N-phenylamino]-triphenylamine (2-TNATA) was used to form a 60 nm-thick hole injection layer, Example Compound or Comparative Example Compound was used to form a 30 nm-thick hole transport layer, 2,5,8,11-tetra-t-butylperylene (TBP) doped by 3% 9,10-di(naphthalene-2-yl)anthracene (ADN) (TBP 3%:ADN 97%) to form a 25 nm-thick emission layer, tris(8-hydroxyquinolinato)aluminum (Alq₃) was used to form a 25 nm-thick electron transport layer, The compound lithium fluoride (LiF) was used to form a 1 nm-thick electron injection layer, and the element Al was used to form a 100 nm-thick second electrode. Each layer was formed by a deposition method in a vacuum atmosphere.

Compounds used for manufacturing the light emitting devices of Examples and Comparative Examples are disclosed below. The compounds below are known materials, and commercial products were subjected to sublimation purification and used to manufacture the devices.

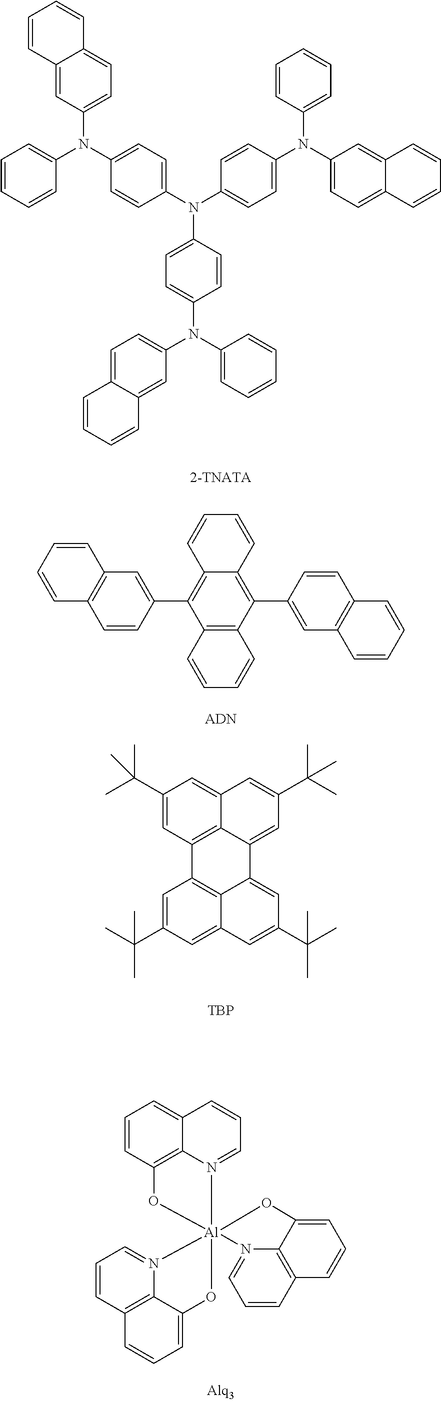

Experimental Example

The device efficiencies of the light emitting devices manufactured by using Example Compounds A3, A36, A45, A50, A54, A74, B4, B99, B107, B138, B151, B263, C30, C62, C114, C148, C190, A59, C222, and C259 as described above and Comparative Example Compounds R1 to R17 were evaluated. The evaluation results are shown in Table 1 below. In the evaluation of the device, luminous efficiencies and lifespans in percent (%) of the light emitting devices were measured at a current density of 10 milliamp per centimeter squared (mA/cm²) and listed. The devices were measured by using a source meter (sold under the trade designation Keithley MU 236, by Tektronix, Inc., of Beaverton, Oreg.) and a luminance meter sold under the trade designation PR650 from Photo Research Inc. of Los Angeles, Calif. In addition, luminous efficiencies and lifespans are represented as a comparative value when efficiency and lifespan of Comparative Example 1-1 are considered 10000.

TABLE 1

| Device | | | |
|---|---|---|---|
| manufacturing | Hole transport layer | Luminous efficiency | Lifespan |
| examples | compound | (%) | (LT₅₀, %) |
| Example 1-1 | Example Compound | 134% | 153% |
| | A3 | | |
| Example 1-2 | Example Compound | 132% | 158% |
| | A36 | | |
| Example 1-3 | Example Compound | 142% | 149% |
| | A45 | | |
| Example 1-4 | Example Compound | 145% | 144% |
| | A50 | | |
| Example 1-5 | Example Compound | 134% | 149% |
| | A54 | | |
| Example 1-6 | Example Compound | 135% | 150% |
| | A74 | | |
| Example 1-7 | Example Compound | 136% | 154% |
| | B4 | | |
| Example 1-8 | Example Compound | 134% | 150% |
| | B99 | | |
| Example 1-9 | Example Compound | 133% | 158% |
| | B107 | | |
| Example 1-10 | Example Compound | 140% | 155% |
| | B138 | | |
| Example 1-11 | Example Compound | 144% | 145% |
| | B151 | | |
| Example 1-12 | Example Compound | 145% | 155% |
| | B263 | | |
| Example 1-13 | Example Compound | 140% | 157% |
| | C30 | | |
| Example 1-14 | Example Compound | 137% | 160% |
| | C62 | | |
| Example 1-15 | Example Compound | 138% | 156% |
| | C114 | | |
| Example 1-16 | Example Compound | 141% | 144% |
| | C148 | | |
| Example 1-17 | Example Compound | 142% | 146% |
| | C190 | | |
| Example 1-18 | Example Compound | 140% | 152% |
| | A59 | | |
| Example 1-19 | Example Compound | 144% | 149% |
| | C222 | | |
| Example 1-20 | Example Compound | 148% | 158% |
| | C259 | | |
| Comparative | Comparative | 100% | 100% |
| Example 1-1 | Example Compound | | |
| | R1 | | |
| Comparative | Comparative | 93% | 102% |
| Example 1-2 | Example Compound | | |
| | R2 | | |
| Comparative | Comparative | 86% | 88% |
| Example 1-3 | Example Compound | | |
| | R3 | | |
| Comparative | Comparative | 102% | 94% |
| Example 1-4 | Example Compound | | |
| | R4 | | |
| Comparative | Comparative | 103% | 103% |
| Example 1-5 | Example Compound | | |
| | R5 | | |
| Comparative | Comparative | 91% | 78% |
| Example 1-6 | Example Compound | | |
| | R6 | | |
| Comparative | Comparative | 104% | 98% |
| Example 1-7 | Example Compound | | |
| | R7 | | |
| Comparative | Comparative | 97% | 100% |
| Example 1-8 | Example Compound | | |
| | R8 | | |
| Comparative | Comparative | 87% | 61% |
| Example 1-9 | Example Compound | | |
| | R9 | | |
| Comparative | Comparative | 79% | 75% |
| Example 1-10 | Example Compound | | |
| | R10 | | |
| Comparative | Comparative | 66% | 68% |
| Example 1-11 | Example Compound | | |
| | R11 | | |
| Comparative | Comparative | 99% | 96% |
| Example 1-12 | Example Compound | | |
| | R12 | | |
| Comparative | Comparative | 93% | 90% |
| Example 1-13 | Example Compound | | |
| | R13 | | |
| Comparative | Comparative | 105% | 106% |
| Example 1-14 | Example Compound | | |
| | R14 | | |
| Comparative | Comparative | 80% | 66% |
| Example 1-15 | Example Compound | | |
| | R15 | | |
| Comparative | Comparative | 103% | 98% |
| Example 1-16 | Example Compound | | |
| | R16 | | |
| Comparative | Comparative | 101% | 89% |
| Example1-17 | Example Compound | | |
| | R17 | | |

Referring to the results of Table 1, Examples of the light emitting devices in which the nitrogen-containing compounds according to examples of the invention are used as a hole transport layer material have significantly and unexpectedly improved luminous efficiencies and lifespan compared to Comparative Examples.

Although not wanting to be bound by theory, Example Compounds may have a structure in which the carbazole substituent is linked to a benzofuroindole or benzothienoindole skeleton at the position of the nitrogen atom of the carbazole substituent via a linker. Alternatively, the nitrogen-containing compound may have a structure in which the carbazole substituent is linked to a benzofuroindole or benzothienoindole skeleton at the position of one carbon atom of the benzene rings of the carbazole substituent via a linker, or a structure in which the carbazole substituent is directly linked to a benzofuroindole or benzothienoindole skeleton at the position of one carbon atom of the benzene rings of the carbazole substituent. Thus, Example Compounds have improved hole transport ability of the entire molecule, the electron resistance and lowest triplet excitation energy may be increased, and thus when Example Compounds are applied to the light emitting device, efficiency and a service life of the light emitting device are improved. In particular, when the light emitting device of an example includes a nitrogen-containing compound made according to the principles and embodiments of the invention as a hole transport layer material of the light emitting device, efficiency and a lifespan of the light emitting device is improved.

Comparative Example Compound R1 and R2 included in Comparative Examples 1-1 and 1-2, respectively, are compounds which do not include a benzofuroindole or benzothienoindole skeleton, Comparative Example Compound R3 included in Comparative Example 1-3 is a compound which includes an indenoindole skeleton instead of the benzofuroindole skeleton, and thus hole transport properties and electron resistance are not sufficient. Accordingly, it may be confirmed that the devices of Comparative Examples 1-1 to 1-3 have decreased luminous efficiencies and lifespans compared to Examples. Comparative Example Compounds R4 and R5 included in Comparative Examples 1-4 and 1-5, respectively, are compounds which have an additional condensed structure in a benzofuroindole or carbazole skeleton, and the planarity of the entire molecule increases, and thus when the deposition temperature increases, materials are decomposed and layer-forming properties are reduced. Accordingly, it may be confirmed that the devices of Comparative Examples 1-4 and 1-5 have luminous efficiencies and lifespans lower than those of Examples have. Although not wanting to be bound by theory, Comparative Example Compound R6 included in Comparative Example 1-6 is a compound in which sp3 hybrid carbon atom parts are present in a benzofuroindole skeleton, and the sp3 hybrid carbon atom parts are unstable under a high temperature condition. Accordingly, it may be confirmed that the device of Comparative Example 1-6 has luminous efficiency and lifespan lower than those of Examples have, and in particular, the lifespan is significantly reduced. Comparative Example Compound R7 included in Comparative Example 1-7 is a compound in which a benzofuroindole skeleton and a carbazole group are connected via an o,o-biphenyl linking group, that is, 2,2′-biphenyl linking group, the 2,2′-biphenyl linking group increases the steric volume, and thus the stability of the compound is reduced. Accordingly, it may be confirmed that the device of Comparative Example 1-7 has luminous efficiency and lifespan lower than those of inventive Examples. Comparative Example Compounds R8 to R11, and R15 included in Comparative Examples 1-8 to 1-11, and 1-15, respectively, are compounds which further include a heteroaryl group containing a plurality of heteroatoms in addition to a benzofuroindole or a benzothienoindole skeleton, or include a cyano group that is an electron-withdrawing substituent, and the charge balance of the compound is broken. Accordingly, it may be confirmed that the devices of Comparative Examples 1-8 to 1-11, and 1-15 have luminous efficiencies and lifespans lower than those of Examples have. Comparative Example Compounds R12 and R13 included in Comparative Examples 1-12 and 1-13 are compounds containing amine groups, and the charge balance of the compound is broken. Accordingly, it may be confirmed that the devices of Comparative Examples 1-12 and 1-13 have luminous efficiencies and lifespans lower than those of Examples have. As for Comparative Example Compound R14 included in Comparative Example 1-14, a carbazole substituent is linked to a benzofuroindole skeleton via a linker, but there are no other substituents such as an aryl group, a heteroaryl group, and a silyl group other than the carbazole substituent, and thus a charge resistance is insignificantly improved. Accordingly, it may be confirmed that the device of Comparative Example 1-14 has luminous efficiency and lifespan lower than those of the inventive Examples. As for Comparative Example Compound R16 included in Comparative Example 1-16, a carbazole substituent is linked to a benzofuroindole skeleton, but is directly linked at the nitrogen atom position of the carbazole substituent, and thus charges are too weighted towards one side, thereby reducing the stability of materials during driving the device. Accordingly, it may be confirmed that the device of Comparative Example 1-16 has luminous efficiency and lifespan lower than those of Examples have. As for Comparative Example Compound R17 included in Comparative Example 1-17, a carbazole substituent is linked to a benzofuroindole skeleton, but two carbazole groups are linked to the benzofuroindole skeleton, and thus the charge balance of the compound is broken. Accordingly, it may be confirmed that the device of Comparative Example 1-17 has luminous efficiency and lifespan lower than those of Examples have.

(2) Manufacture and Evaluation of Light Emitting Device in which Nitrogen-Containing Compound is Applied as Hole Transport Layer Material and Phosphorescence Emission Compound is Used in Emission Layer

Manufacture of Light Emitting Device

Compounds A3, A36, A45, A50, A54, A74, B4, B99, B107, B138, B151, B263, C30, C62, C114, C148, C190, A59, C222, and C259 as described above were used as a hole transport layer material to manufacture the light emitting devices of Examples 2-1 to 2-20, respectively.

Comparative Example Compounds R1 to R17 as described above were used to manufacture the devices of Comparative Examples.

The light emitting device of an example including the nitrogen-containing compound of an example in a hole transport layer was manufactured as follows. Examples 2-1 to 2-20 correspond to the light emitting devices manufactured by using Compounds A3, A36, A45, A50, A54, A74, B4, B99, B107, B138, B151, B263, C30, C62, C114, C148, C190, A59, C222, and C259 which are Example Compounds as described above as a hole transport layer material, respectively. Comparative Examples 2-1 to 2-17 correspond to the light emitting devices manufactured by using Comparative Example Compounds R1 to R17 as a hole transport layer material, respectively.

The compound ITO was used to form a 120 nm-thick first electrode, N,N′-di(naphthalene-1-yl)-N,N′-diphenyl-benzidine (NPB) was used to form a 40 nm-thick hole injection layer, Example Compound or Comparative Example Compound was used to form a 10 nm-thick hole transport layer, tris(2-phenylpyridine)iridium(III) (Ir(ppy)₃) was doped by 5% of 4,4′-bis(carbazol-9-yl)biphenyl (CBP) (Ir(ppy)₃ 3%:CBP 97%) to form a 30 nm-thick emission layer, 4,7-diphenyl-1,10-phenanthroline (BPhen) was used to form a 50 nm-thick electron transport layer, the compound LiF was used to form a 0.1 nm-thick electron injection layer, and the element A1 was used to form a 110 nm-thick second electrode. Each layer was formed by a deposition method in a vacuum atmosphere.

Compounds used for manufacturing the light emitting devices of Examples and Comparative Examples are disclosed below. The compounds below are known materials, and commercial products were subjected to sublimation purification and used to manufacture the devices.

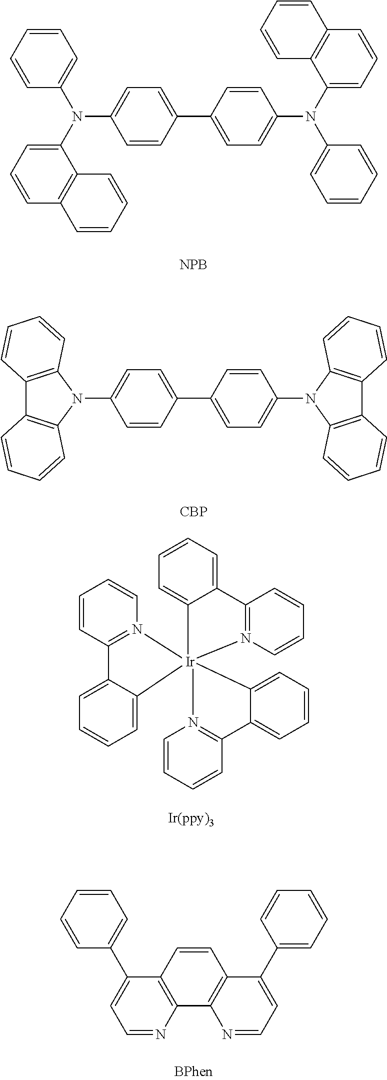

### Experimental Example

The device efficiencies of the light emitting devices manufactured by using Example Compounds A3, A36, A45, A50, A54, A74, B4, B99, B107, B138, B151, B263, C30, C62, C114, C148, C190, A59, C222, and C259 as described above and Comparative Example Compounds R1 to R17 were evaluated. The evaluation results are shown in Table 2 below. In the evaluation of the device, luminous efficiencies and lifespans of the light emitting devices were measured at a current density of 10 mA/cm² and depicted below, and substantially the same equipment and procedures were used as described above. In addition, luminous efficiencies and lifespans are represented as a comparative value when efficiency and lifespan of Comparative Example 2-1 are considered 100%.

TABLE 2

| Device | | | |
|---|---|---|---|
| manufacturing | Hole transport layer | Luminous efficiency | Lifespan |
| examples | compound | (%) | (LT₅₀, %) |
| Example 2-1 | Example Compound | 136% | 155% |
| | A3 | | |
| Example 2-2 | Example Compound | 134% | 158% |
| | A36 | | |
| Example 2-3 | Example Compound | 150% | 148% |
| | A45 | | |
| Example 2-4 | Example Compound | 151% | 146% |
| | A50 | | |
| Example 2-5 | Example Compound | 145% | 146% |
| | A54 | | |
| Example 2-6 | Example Compound | 147% | 145% |
| | A74 | | |
| Example 2-7 | Example Compound | 139% | 150% |
| | B4 | | |
| Example 2-8 | Example Compound | 135% | 148% |
| | B99 | | |
| Example 2-9 | Example Compound | 137% | 157% |
| | B107 | | |
| Example 2-10 | Example Compound | 142% | 160% |
| | B138 | | |
| Example 2-11 | Example Compound | 144% | 149% |
| | B151 | | |
| Example 2-12 | Example Compound | 140% | 155% |
| | B263 | | |
| Example 2-13 | Example Compound | 146% | 154% |
| | C30 | | |
| Example 2-14 | Example Compound | 147% | 157% |
| | C62 | | |
| Example 2-15 | Example Compound | 142% | 156% |
| | C114 | | |
| Example 2-16 | Example Compound | 149% | 145% |
| | C148 | | |
| Example 2-17 | Example Compound | 150% | 160% |
| | C190 | | |
| Example 2-18 | Example Compound | 146% | 159% |
| | A59 | | |
| Example 2-19 | Example Compound | 151% | 155% |
| | C222 | | |
| Example 2-20 | Example Compound | 155% | 150% |
| | C259 | | |
| Comparative | Comparative | 100% | 100% |
| Example 2-1 | Example Compound | | |
| | R1 | | |
| Comparative | Comparative | 94% | 105% |
| Example 2-2 | Example Compound | | |
| | R2 | | |
| Comparative | Comparative | 89% | 90% |
| Example 2-3 | Example Compound | | |
| | R3 | | |
| Comparative | Comparative | 105% | 97% |
| Example 2-4 | Example Compound | | |
| | R4 | | |
| Comparative | Comparative | 102% | 104% |
| Example 2-5 | Example Compound | | |
| | R5 | | |
| Comparative | Comparative | 94% | 81% |
| Example 2-6 | Example Compound | | |
| | R6 | | |
| Comparative | Comparative | 99% | 91% |
| Example 2-7 | Example Compound | | |
| | R7 | | |
| Comparative | Comparative | 94% | 94% |
| Example 2-8 | Example Compound | | |
| | R8 | | |
| Comparative | Comparative | 84% | 61% |
| Example 2-9 | Example Compound | | |
| | R9 | | |
| Comparative | Comparative | 75% | 69% |
| Example 2-10 | Example Compound | | |
| | R10 | | |
| Comparative | Comparative | 70% | 64% |
| Example 2-11 | Example Compound | | |
| | R11 | | |
| Comparative | Comparative | 97% | 99% |
| Example 2-12 | Example Compound | | |
| | R12 | | |
| Comparative | Comparative | 93% | 96% |
| Example 2-13 | Example Compound | | |
| | R13 | | |
| Comparative | Comparative | 103% | 109% |
| Example 2-14 | Example Compound | | |
| | R14 | | |
| Comparative | Comparative | 77% | 63% |
| Example 2-15 | Example Compound | | |
| | R15 | | |
| Comparative | Comparative | 101% | 102% |
| Example 2-16 | Example Compound | | |
| | R16 | | |
| Comparative | Comparative | 99% | 92% |
| Example 2-17 | Example Compound | | |
| | R17 | | |

Table 2 shows that Examples of the light emitting devices in which the nitrogen-containing compounds according to examples of the invention are used as a hole transport layer material have significantly and unexpectedly improved luminous efficiencies and lifespans compared to Comparative Examples. It may be confirmed from the results of Table 1, although not wanting to be bound by theory, when being applied as a hole transport layer material of the device including a fluorescence emission compound in an emission layer, the nitrogen-containing compound according to an example may not only improve the luminous efficiency and lifespan of the light emitting device, but like the results of Table 2, when being applied as a hole transport layer material of the device including a phosphorescence emission compound in an emission layer, the nitrogen-containing compound may also improve the luminous efficiency and lifespan of the light emitting device.

In Table 1, the description of Examples 1-1 to 1-20 and Comparative Examples 1-1 to 1-17 may be equally applied to Examples 2-1 to 2-20 and Comparative Examples 2-1 to 2-17.

(3) Manufacture and evaluation of light emitting device in which nitrogen-containing compound is applied as host material

Manufacture of Light Emitting Device

Compounds A3, A36, A45, A50, A54, A74, B4, B99, B107, B138, B151, B263, C30, C62, C114, C148, C190, A59, C222, and C259 as described above were used as a host material to manufacture the light emitting devices of Examples 3-1 to 3-20, respectively.

Comparative Example Compounds R1 to R17 as described above were used to manufacture the devices of Comparative Examples.

The light emitting device of an example including the nitrogen-containing compound of an example in an emission layer was manufactured as follows. Examples 3-1 to 3-20 correspond to the light emitting devices manufactured by using Compounds A3, A36, A45, A50, A54, A74, B4, B99, B107, B138, B151, B263, C30, C62, C114, C148, C190, A59, C222, and C259 which are Example Compounds as described above as a host material of an emission layer, respectively. Comparative Examples 3-1 to 3-17 correspond to the light emitting devices manufactured by using Comparative Example Compounds R1 to R17 as a host material of an emission layer, respectively.

The compound ITO was used to form a 120 nm-thick first electrode, N,N′-di(naphthalene-1-yl)-N,N′-diphenyl-benzidine (NPB) was used to form a 40 nm-thick hole injection layer, 4,4′,4″-tris(N-carbazolyl)triphenylamine (TCTA) was used to form a 10 nm-thick hole transport layer, tris(2-phenylpyridine)iridium(III) (Ir(ppy)₃) was doped by 5% of Example Compound or Comparative Example Compound to form a 30 nm-thick emission layer, 4,7-diphenyl-1,10-phenanthroline (BPhen) was used to form a 50 nm-thick electron transport layer, the compound LiF was used to form a 0.1 nm-thick electron injection layer, and the element A1 was used to form a 110 nm-thick second electrode. Each layer was formed by a deposition method in a vacuum atmosphere.

Compounds used for manufacturing the light emitting devices of Examples and Comparative Examples are disclosed below. The compounds below are known materials, and commercial products were subjected to sublimation purification and used to manufacture the devices.

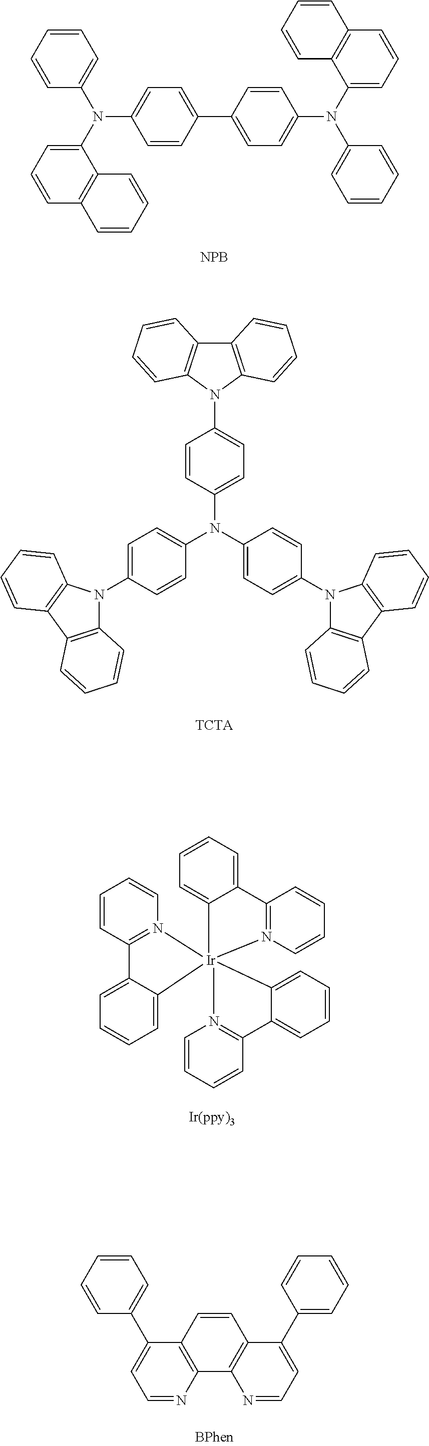

Experimental Example

The device efficiencies of the light emitting devices manufactured by using Example Compounds A3, A36, A45, A50, A54, A74, B4, B99, B107, B138, B151, B263, C30, C62, C114, C148, C190, A59, C222, and C259 as described above and Comparative Example Compounds R1 to R17 were evaluated. The evaluation results are shown in Table 3 below. In the evaluation of the device, luminous efficiencies and lifespans of the light emitting devices were measured at a current density of 10 mA/cm² and depicted below, and substantially the same equipment and procedures were used as described above. In addition, luminous efficiencies and lifespans are represented as a comparative value when efficiency and lifespan of Comparative Example 3-1 are considered 100%.

TABLE 3

| Device | | | |
|---|---|---|---|
| manufacturing | Emission layer host | Luminous efficiency | Lifespan |
| examples | compound | (%) | (LT₅₀, %) |
| Example 3-1 | Example Compound | 133% | 150% |
| | A3 | | |
| Example 3-2 | Example Compound | 135% | 156% |
| | A36 | | |
| Example 3-3 | Example Compound | 149% | 162% |
| | A45 | | |
| Example 3-4 | Example Compound | 152% | 159% |
| | A50 | | |
| Example 3-5 | Example Compound | 141% | 152% |
| | A54 | | |
| Example 3-6 | Example Compound | 143% | 157% |
| | A74 | | |
| Example 3-7 | Example Compound | 148% | 154% |
| | B4 | | |
| Example 3-8 | Example Compound | 137% | 156% |
| | B99 | | |
| Example 3-9 | Example Compound | 139% | 160% |
| | B107 | | |
| Example 3-10 | Example Compound | 147% | 158% |
| | B138 | | |
| Example 3-11 | Example Compound | 149% | 150% |
| | B151 | | |
| Example 3-12 | Example Compound | 146% | 153% |
| | B263 | | |
| Example 3-13 | Example Compound | 150% | 144% |
| | C30 | | |
| Example 3-14 | Example Compound | 139% | 161% |
| | C62 | | |
| Example 3-15 | Example Compound | 136% | 147% |
| | C114 | | |
| Example 3-16 | Example Compound | 135% | 144% |
| | C148 | | |
| Example 3-17 | Example Compound | 141% | 150% |
| | C190 | | |
| Example 3-18 | Example Compound | 138% | 158% |
| | A59 | | |
| Example 3-19 | Example Compound | 139% | 153% |
| | C222 | | |
| Example 3-20 | Example Compound | 145% | 160% |
| | C259 | | |
| Comparative | Comparative | 100% | 100% |
| Example 3-1 | Example Compound | | |
| | R1 | | |
| Comparative | Comparative | 105% | 110% |
| Example 3-2 | Example Compound | | |
| | R2 | | |
| Comparative | Comparative | 90% | 91% |
| Example 3-3 | Example Compound | | |
| | R3 | | |
| Comparative | Comparative | 101% | 90% |
| Example 3-4 | Example Compound | | |
| | R4 | | |
| Comparative | Comparative | 99% | 104% |
| Example 3-5 | Example Compound | | |
| | R5 | | |
| Comparative | Comparative | 94% | 87% |
| Example 3-6 | Example Compound | | |
| | R6 | | |
| Comparative | Comparative | 104% | 104% |
| Example 3-7 | Example Compound | | |
| | R7 | | |
| Comparative | Comparative | 97% | 92% |
| Example 3-8 | Example Compound | | |
| | R8 | | |
| Comparative | Comparative | 94% | 88% |
| Example 3-9 | Example Compound | | |
| | R9 | | |
| Comparative | Comparative | 74% | 65% |
| Example 3-10 | Example Compound | | |
| | R10 | | |
| Comparative | Comparative | 80% | 72% |
| Example 3-11 | Example Compound | | |
| | R11 | | |
| Comparative | Comparative | 87% | 81% |
| Example 3-12 | Example Compound | | |
| | R12 | | |
| Comparative | Comparative | 91% | 89% |
| Example 3-13 | Example Compound | | |
| | R13 | | |
| Comparative | Comparative | 103% | 109% |
| Example 3-14 | Example Compound | | |
| | R14 | | |
| Comparative | Comparative | 85% | 77% |
| Example 3-15 | Example Compound | | |
| | R15 | | |
| Comparative | Comparative | 98% | 105% |
| Example 3-16 | Example Compound | | |
| | R16 | | |
| Comparative | Comparative | 95% | 93% |
| Example 3-17 | Example Compound | | |
| | R17 | | |

Table 3 shows that Examples of the light emitting devices in which the nitrogen-containing compounds according to examples of the invention are used as an emission layer host material have significantly and unexpectedly improved luminous efficiencies and lifespans compared to Comparative Examples in which Comparative Example Compounds are used as a host material. Like the results of Tables 1 and 2, although not wanting to be bound by theory, when the nitrogen-containing compound according to an example is applied as a hole transport layer material of the device, not only does the luminous efficiency and lifespan of the light emitting device improve, but like the results of Table 3, when being applied as a host material of an emission layer, the nitrogen-containing compound may also improve the luminous efficiency and lifespan of the light emitting device. In particular, if the nitrogen-containing compound of an example is applied as a host material of an emission layer, it is possible to make a big difference of T1 level between a dopant material and the host material through a high T1 level thereof, thereby a reverse intersystem crossing is prevented, and thus the device may have high luminous efficiency, and because the nitrogen-containing compound has high chemical stability and charge transport properties, the device may have a long lifespan.

In Table 1, the description of Examples 1-1 to 1-20 and Comparative Examples 1-1 to 1-17 may be equally applied to Examples 3-1 to 3-20 and Comparative Examples 3-1 to 3-17.

Light emitting devices constructed according to the principles and embodiments of the invention exhibit improved device characteristics with high efficiency and a long lifespan. The nitrogen-containing compounds made according to the principles and embodiments of the invention may be included in a hole transport layer or an emission layer of the light emitting device to contribute to high efficiency and a long lifespan of the light emitting device.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. A light emitting device comprising:
a first electrode;
a second electrode facing the first electrode; and
a plurality of organic layers disposed between the first electrode and the second electrode,
wherein at least one of the organic layers comprises a compound including a nitrogen moiety, and
the compound is of Formula 1 below: 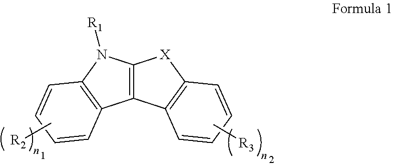
wherein, in Formula 1,
X is O or S,
R₁ to R₃ are each, independently from one another, a hydrogen atom, a deuterium atom, a halogen atom, a nitro group, a substituted or unsubstituted silyl group, a substituted or unsubstituted boron group, a substituted or unsubstituted oxy group, a substituted or unsubstituted thio group, a substituted or unsubstituted carbonyl group, a substituted or unsubstituted alkyl group having 1 to 30 carbon atoms, a substituted or unsubstituted aryl group having 6 to 60 carbon atoms, or a substituted or unsubstituted heteroaryl group 2 to 60 ring-forming carbon atoms,
n₁ and n₂ are each, independently from one another, an integer of 1 to 4, and
any one of Formula 2-1 below or Formula 2-2 below is a substituent of at least one of R₁ to R₃: 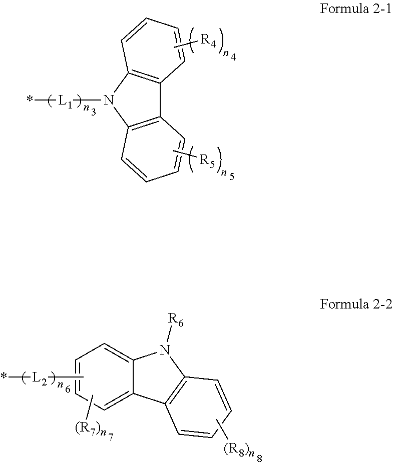
wherein, in Formula 2-1 and Formula 2-2,
L₁ and L₂ are each, independently from one another, a substituted or unsubstituted silyl linking group, a substituted or unsubstituted arylene group having 6 to 60 ring-forming carbon atoms, or a substituted or unsubstituted heteroarylene group having 2 to 60 ring-forming carbon atoms, excluding when each of L₁ and L₂ is a 2,2′-biphenyl linking group, a pyridine linking group, a pyrazine linking group, a quinoline linking group, a quinoxaline linking group, and a triazine linking group,
R₄ to R₈ are each, independently from one another, a hydrogen atom, a deuterium atom, a halogen atom, a nitro group, a substituted or unsubstituted silyl group, a substituted or unsubstituted boron group, a substituted or unsubstituted oxy group, a substituted or unsubstituted thio group, a substituted or unsubstituted carbonyl group, a substituted or unsubstituted alkyl group having 1 to 30 carbon atoms, a substituted or unsubstituted aryl group having 6 to 60 ring-forming carbon atoms, or a substituted or unsubstituted heteroaryl group having 2 to 60 ring-forming carbon atoms,
n₃ and n₇ are each, independently from one another, an integer of 1 to 3,
n₄, n₅, and n₈ are each, independently from one another, an integer of 1 to 4,
n₆ is an integer of 0 to 3, [symbol]
is a position linked to Formula 1,
when a substituent of Formula 2-1 is substituted in the compound of Formula 1, L₁ is a substituted or unsubstituted arylene group having 6 to 60 ring-forming carbon atoms, and n₃ is 1,
at least one of R₂ to R₅ is a substituted or unsubstituted silyl group, a substituted or unsubstituted aryl group having 6 to 60 ring-forming carbon atoms, or a substituted or unsubstituted heteroaryl group having 2 to 60 ring-forming carbon atoms.

2. The light emitting device of claim 1, wherein the organic layers comprise:
a hole transport region disposed on the first electrode;
an emission layer disposed on the hole transport region; and
an electron transport region disposed on the emission layer, and
the hole transport region comprises the compound.

3. The light emitting device of claim 2, wherein the hole transport region comprises:
a hole injection layer disposed on the first electrode; and
a hole transport layer disposed on the hole injection layer, and
the hole transport layer comprises the compound.

4. The light emitting device of claim 2, wherein the hole transport region comprises a plurality of organic layers, and
an organic layer adjacent to the emission layer comprises the compound.

5. The light emitting device of claim 1, wherein the organic layers comprise:
a hole transport region disposed on the first electrode;
an emission layer disposed on the hole transport region; and
an electron transport region disposed on the emission layer, and
wherein the emission layer comprises a host and a dopant, and
the host comprises the compound.

6. The light emitting device of claim 1,
wherein the nitrogen compound of Formula 1 is any one of Formula 1-1 to Formula 1-3 below: 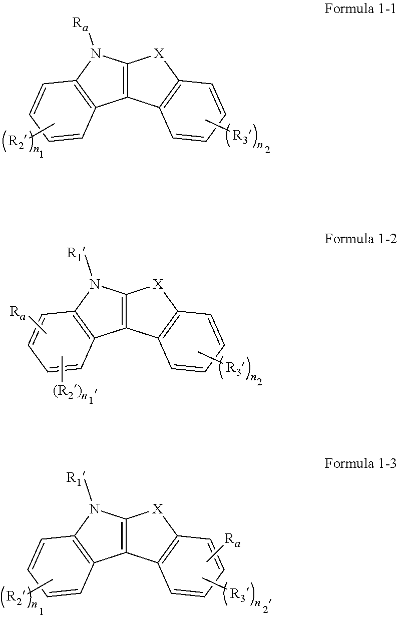
wherein, in Formula 1-1 to Formula 1-3,
Rₐ is a substituent of Formula 2-1 or Formula 2-2,
R₁′, R₂′, and R₃′ are each, independently from one another, a hydrogen atom, a deuterium atom, a halogen atom, a nitro group, a substituted or unsubstituted silyl group, a substituted or unsubstituted boron group, a substituted or unsubstituted oxy group, a substituted or unsubstituted thio group, a substituted or unsubstituted carbonyl group, a substituted or unsubstituted alkyl group having 1 to 30 carbon atoms, a substituted or unsubstituted aryl group having 6 to 60 ring-forming carbon atoms, or a substituted or unsubstituted heteroaryl group having 2 to 60 ring-forming carbon atoms, excluding when each of R₁′, R₂′, and R₃′ is of Formula 2-1 or Formula 2-2,
n₁′ and n₂′ are each, independently from one another, an integer of 1 to 3, and
X, n₁ and n₂ have, independently from one another, the same meaning as in Formula 1.

7. The light emitting device of claim 1, wherein the substituent of Formula 2-1 is of Formula 2-1-1 below: 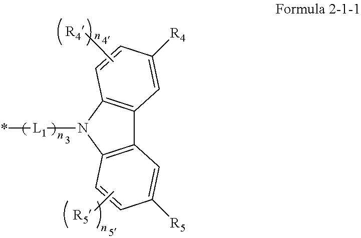
wherein, in Formula 2-1-1,
R₄′ and R₅′ are each, independently from one another, a hydrogen atom or a deuterium atom,
n₄′ and n₅′ are each, independently from one another, an integer of 1 to 3, and
L₁, n₃, R₄, and R₅ have, independently from one another, the same meaning as in Formula 2-1.

8. The light emitting device of claim 1, wherein the substituent of Formula 2-2 is any one of Formula 2-2-1 to Formula 2-2-4 below: 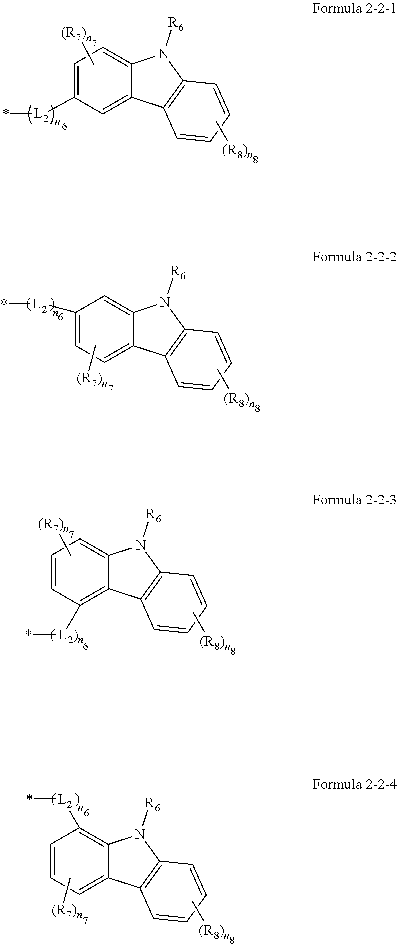
wherein, in Formula 2-2-1 and Formula 2-2-4,
L₂, R₆ to R₈, and n₆ to n₈ have, independently from one another, the same meaning as in Formula 2-2 in claim 1.

9. The light emitting device of claim 1, wherein L₁ and L₂ are each, independently from one another, any one of Formula 3-1 to Formula 3-12 below: 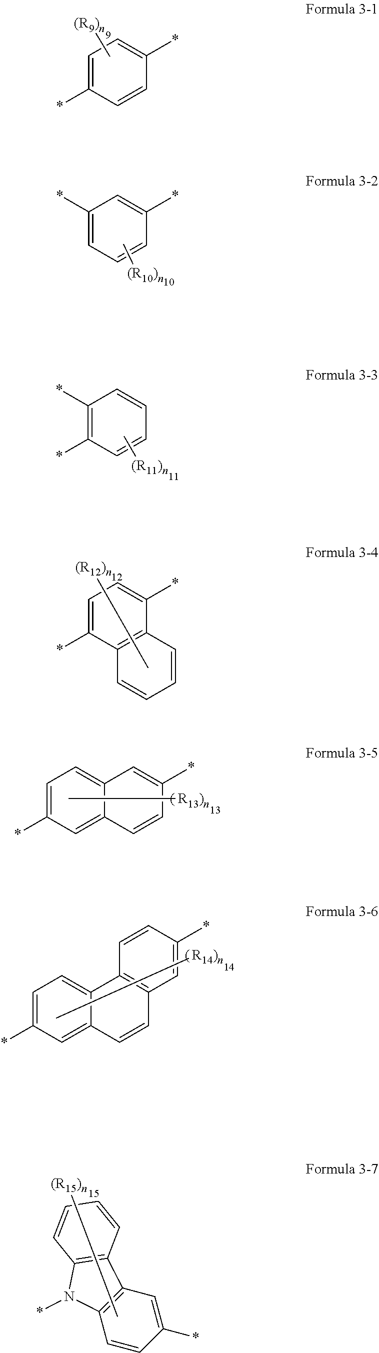 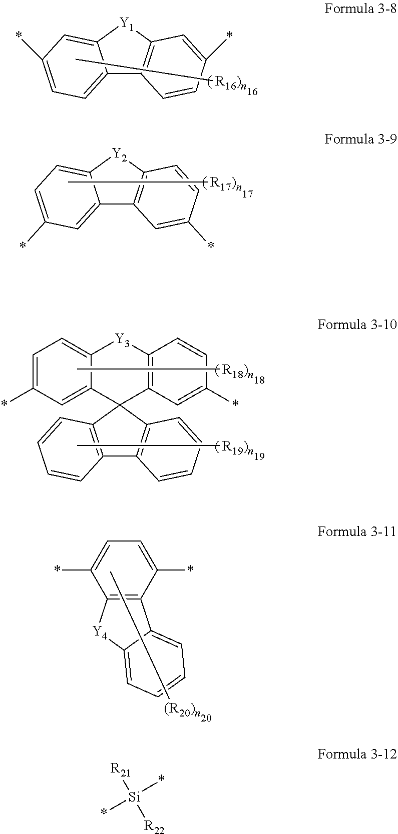
wherein, in Formula 3-1 to Formula 3-12,
Y₁ to Y₄ are each, independently from one another, NR_{b}, O or S,
R_{b} and R₉ to R₂₂ are each, independently from one another, a hydrogen atom, a deuterium atom, a halogen atom, a nitro group, a substituted or unsubstituted silyl group, a substituted or unsubstituted boron group, a substituted or unsubstituted oxy group, a substituted or unsubstituted thio group, a substituted or unsubstituted carbonyl group, a substituted or unsubstituted alkyl group having 1 to 30 carbon atoms, a substituted or unsubstituted aryl group having 6 to 60 carbon atoms, or a substituted or unsubstituted heteroaryl group 2 to 60 ring-forming carbon atoms,
n₉ to n₁₁ are each, independently from one another, an integer of 1 to 4,
n₁₂, n₁₃, n₁₅ to n₁₈, and n₂₀ are each, independently from one another, an integer of 1 to 6,
n₁₄ and n₁₉ are each, independently from one another, an integer of 1 to 8, and [symbol]
is a position linked to Formula 1 and a carbazole moiety of Formula 2-1 and Formula 2-2.

10. The light emitting device of claim 1, wherein R₁ to R₈ are each, independently from one another, a hydrogen atom, a deuterium atom, a halogen atom, a substituted or unsubstituted silyl group, a substituted or unsubstituted methyl group, a substituted or unsubstituted t-butyl group, a substituted or unsubstituted phenyl group, a substituted or unsubstituted naphthyl group, a substituted or unsubstituted biphenyl group, a substituted or unsubstituted terphenyl group, a substituted or unsubstituted phenanthrenyl group, a substituted or unsubstituted carbazole group, a substituted or unsubstituted dibenzofuran group, a substituted or unsubstituted dibenzothiophene group, a substituted or unsubstituted phenoxazine group, or a substituted or unsubstituted acridine group.

11. The light emitting device of claim 1, wherein in Formula 1, when R₁ is a substituent of Formula 2-1 or Formula 2-2,
R₂ and R₃ are each, independently from one another, a hydrogen atom, a deuterium atom, a halogen atom, a substituted or unsubstituted methyl group, or a substituted or unsubstituted phenyl group.

12. The light emitting device of claim 1, wherein in Formula 1, when any one of R₂ or R₃ is a substituent of Formula 2-1 or Formula 2-2,
R₁ is a substituted or unsubstituted phenyl group, a substituted or unsubstituted biphenyl group, a substituted or unsubstituted terphenyl group, or a substituted or unsubstituted naphthyl group.

13. The light emitting device of claim 1, further comprising a capping layer disposed on the second electrode, wherein the capping layer has a refractive index of about 1.6 or more.

14. The light emitting device of claim 1, wherein the compound of Formula 1 comprises at least one compound of Compound Group 1 to Compound Group 3 below:

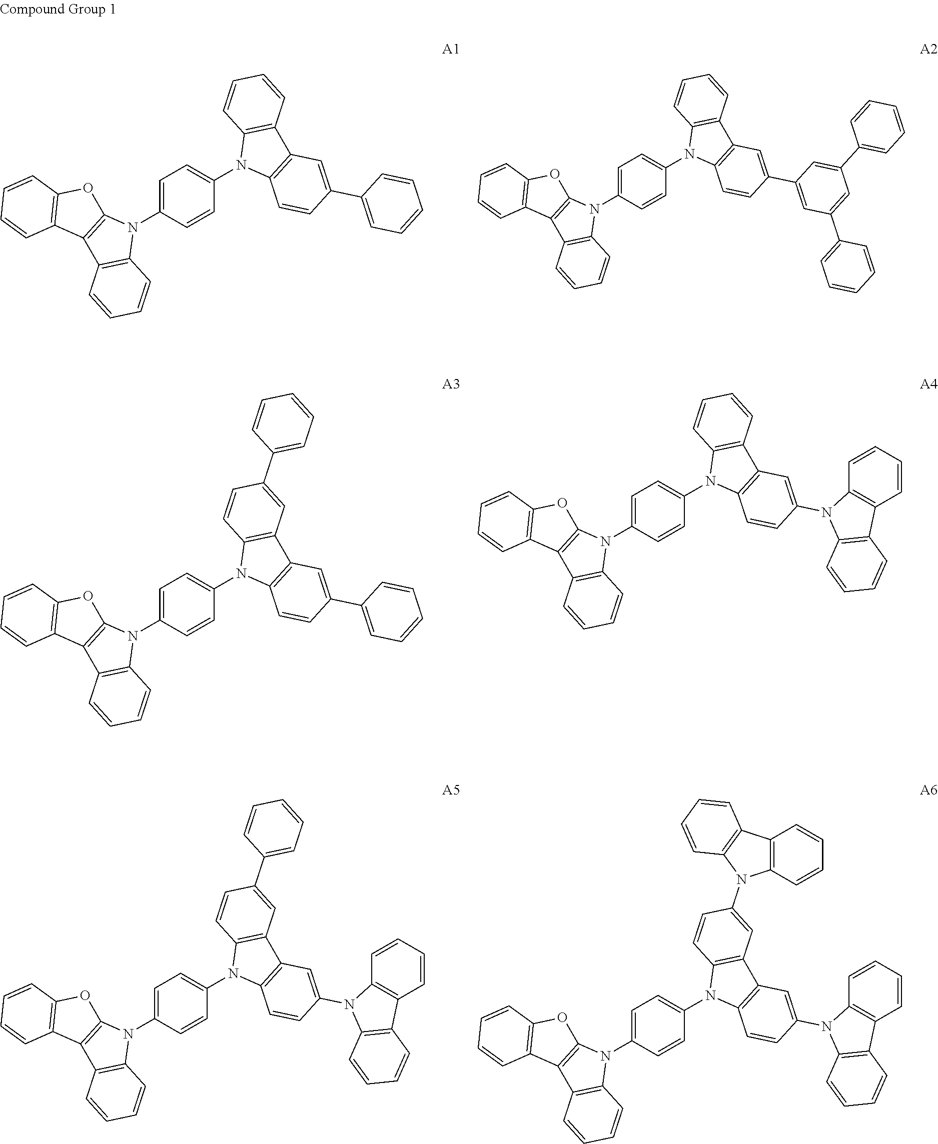

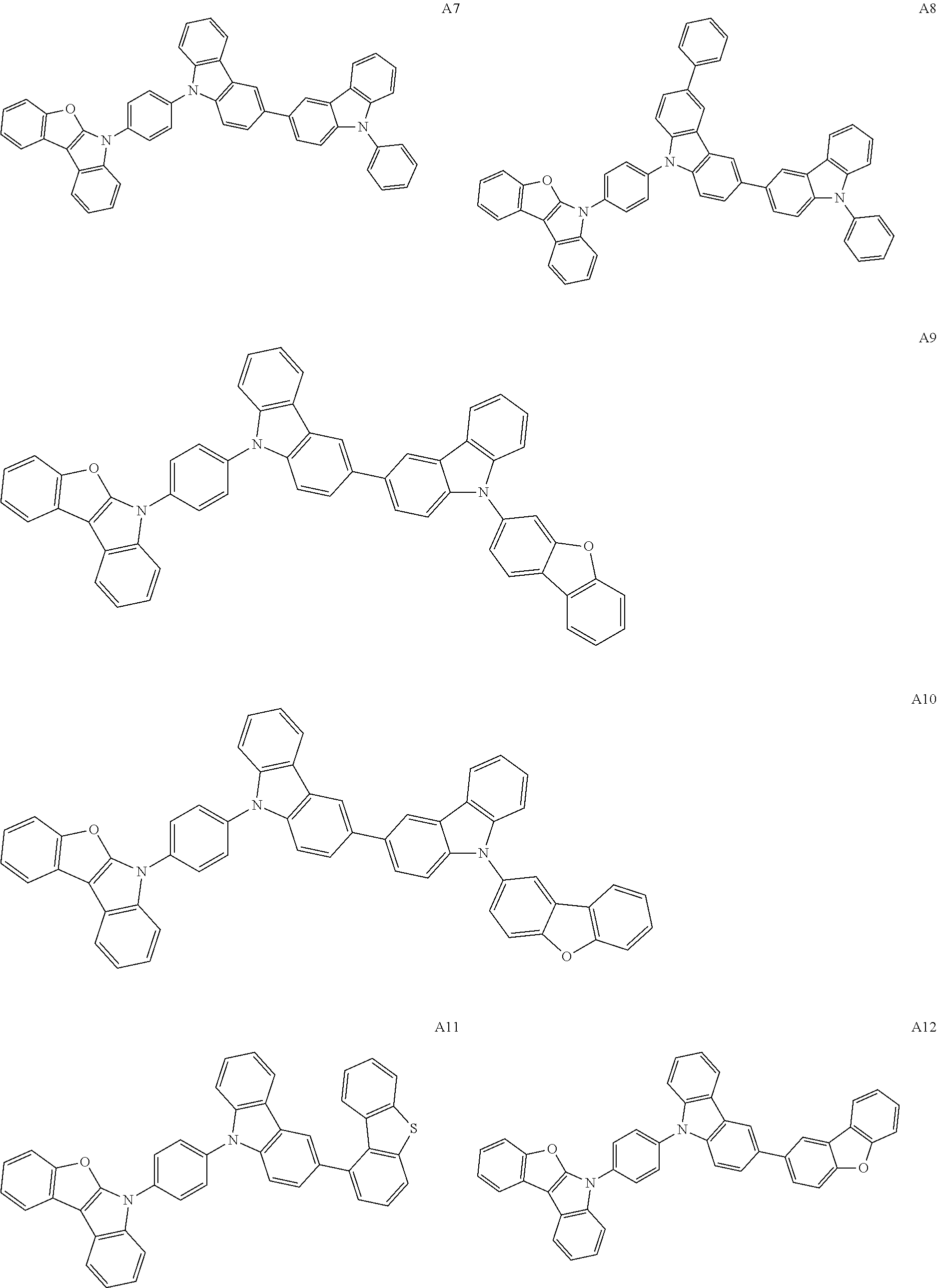

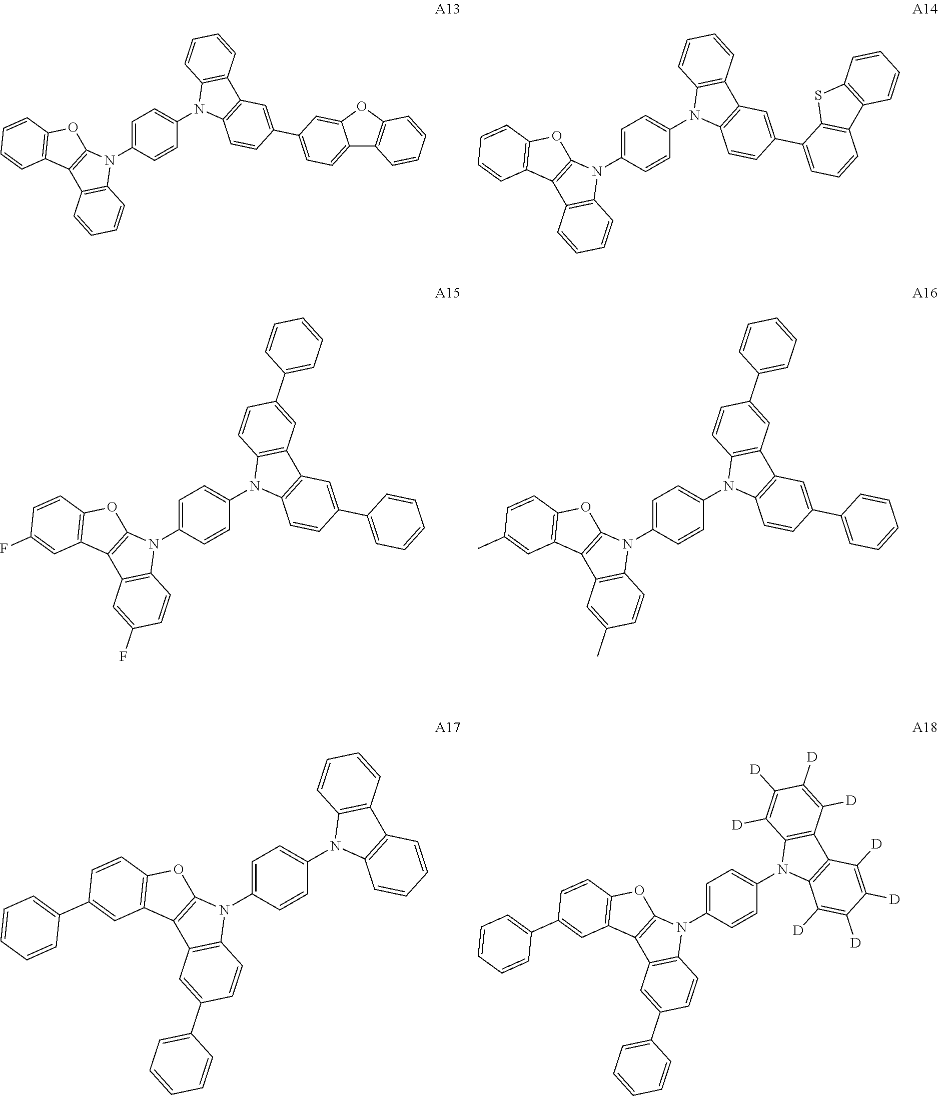

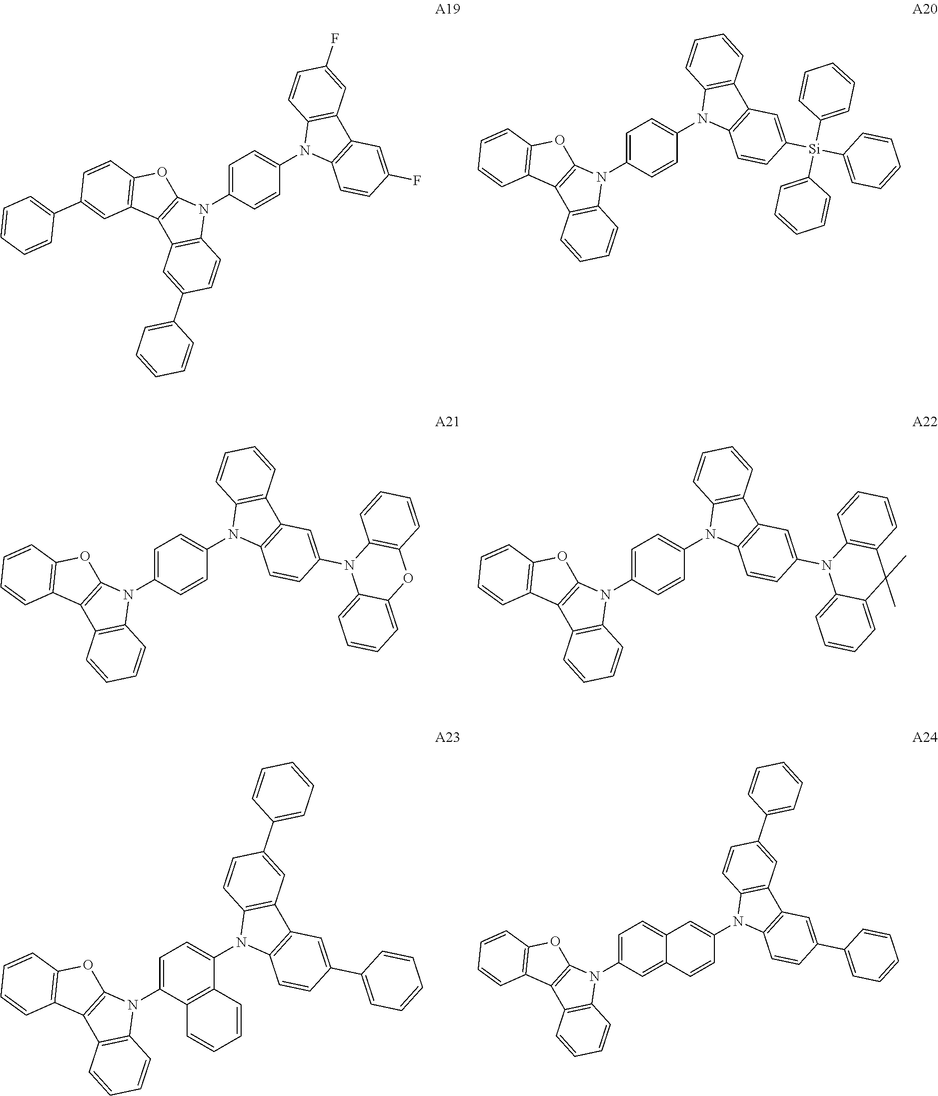

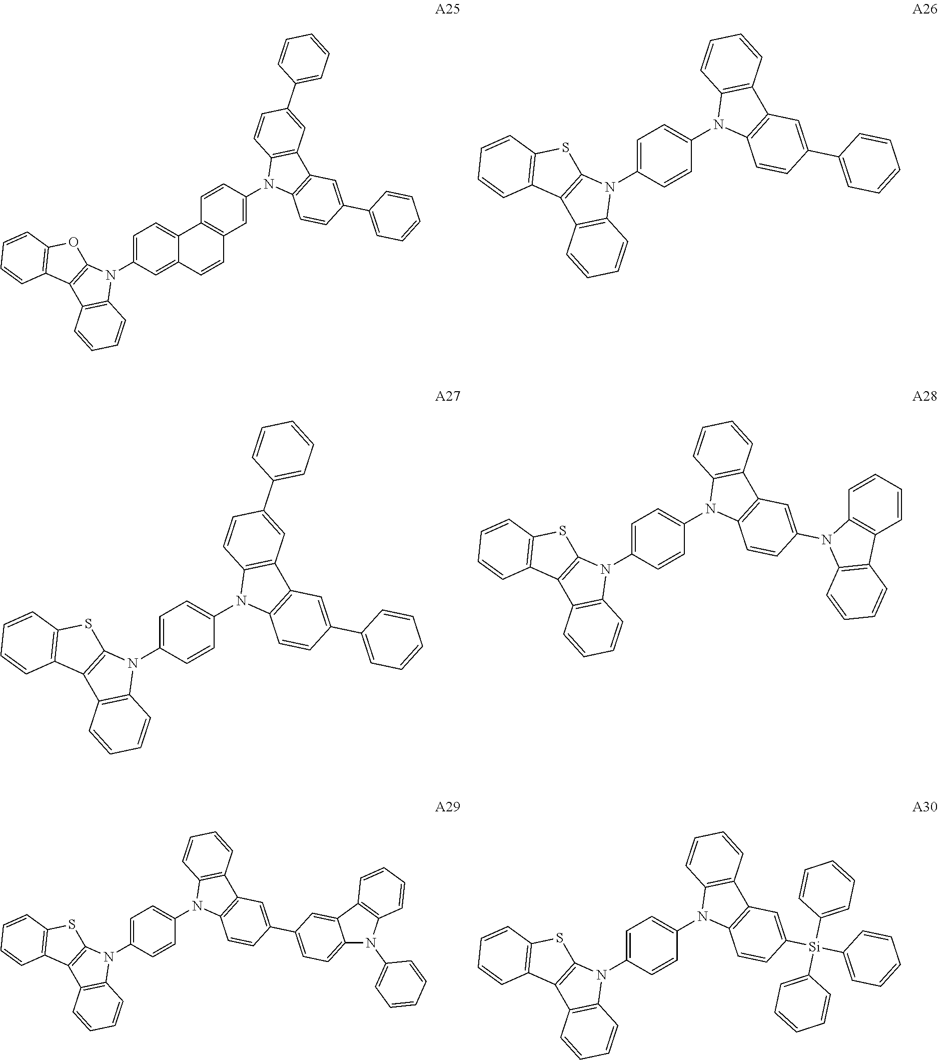

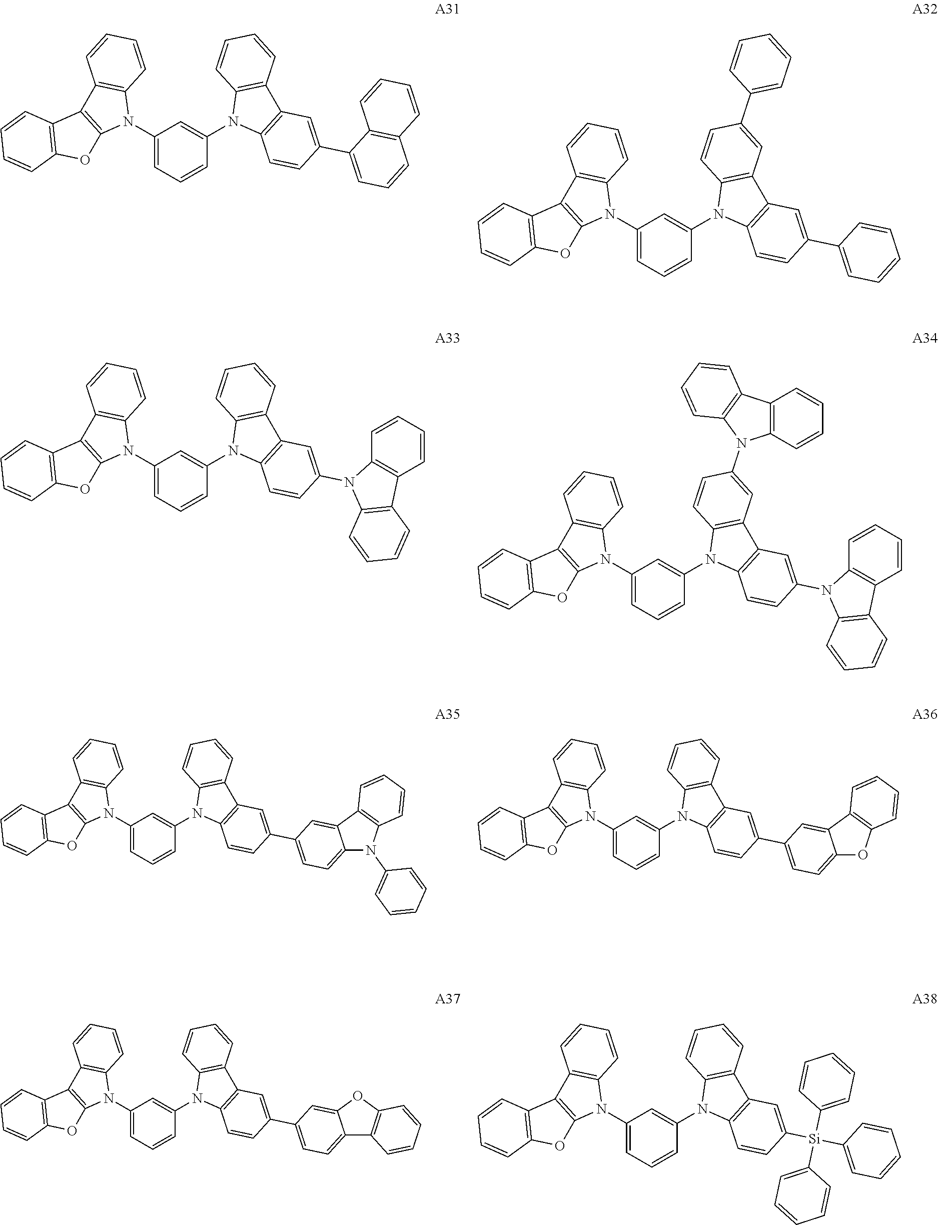

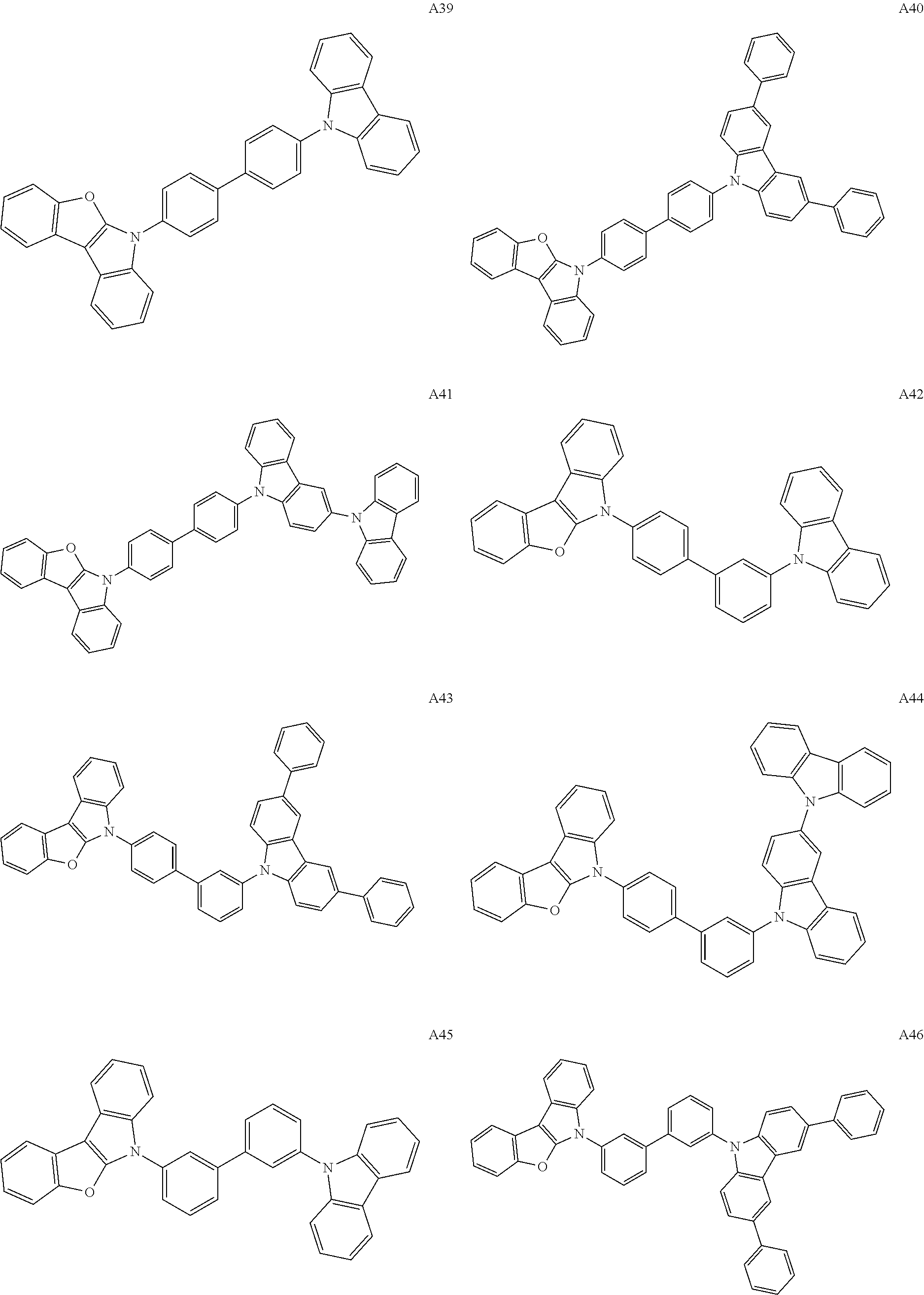

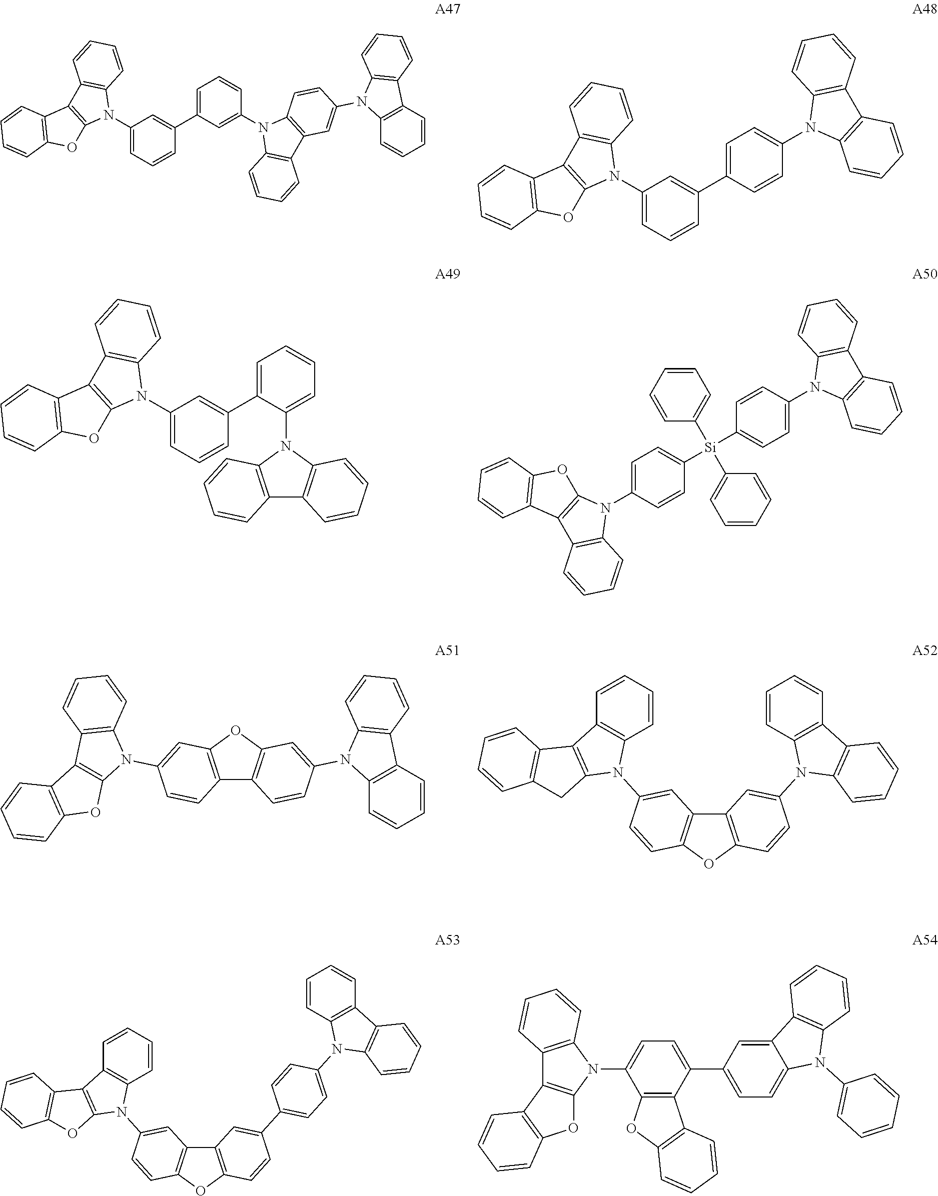

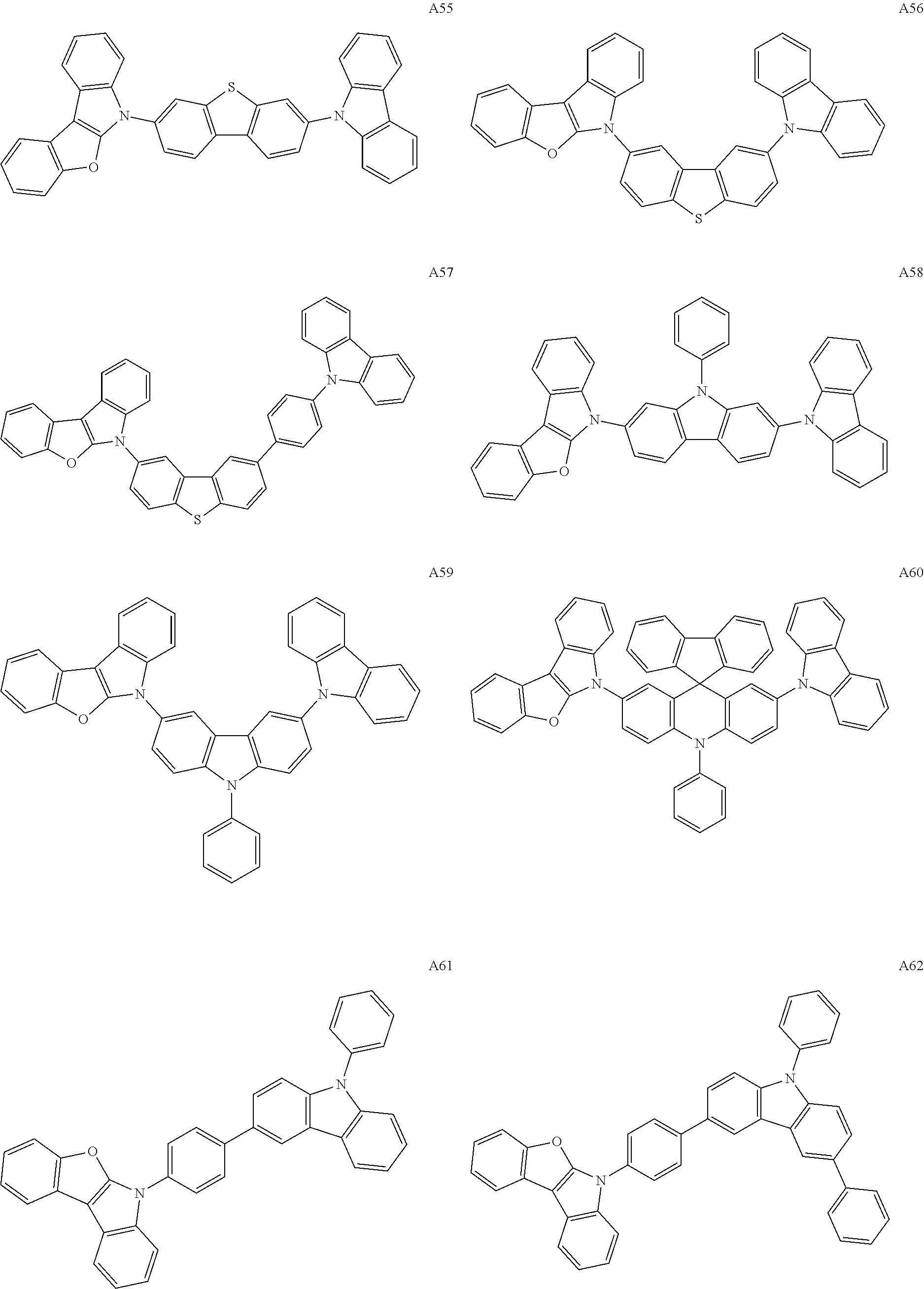

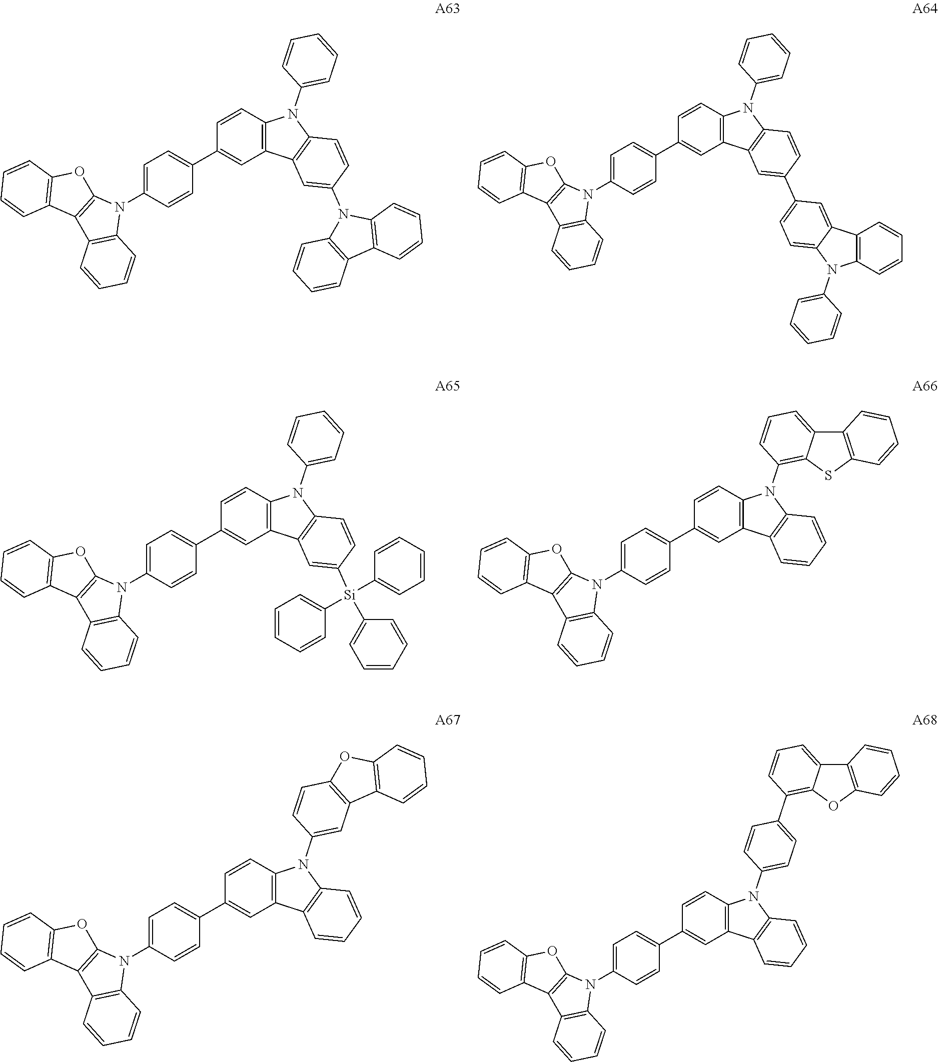

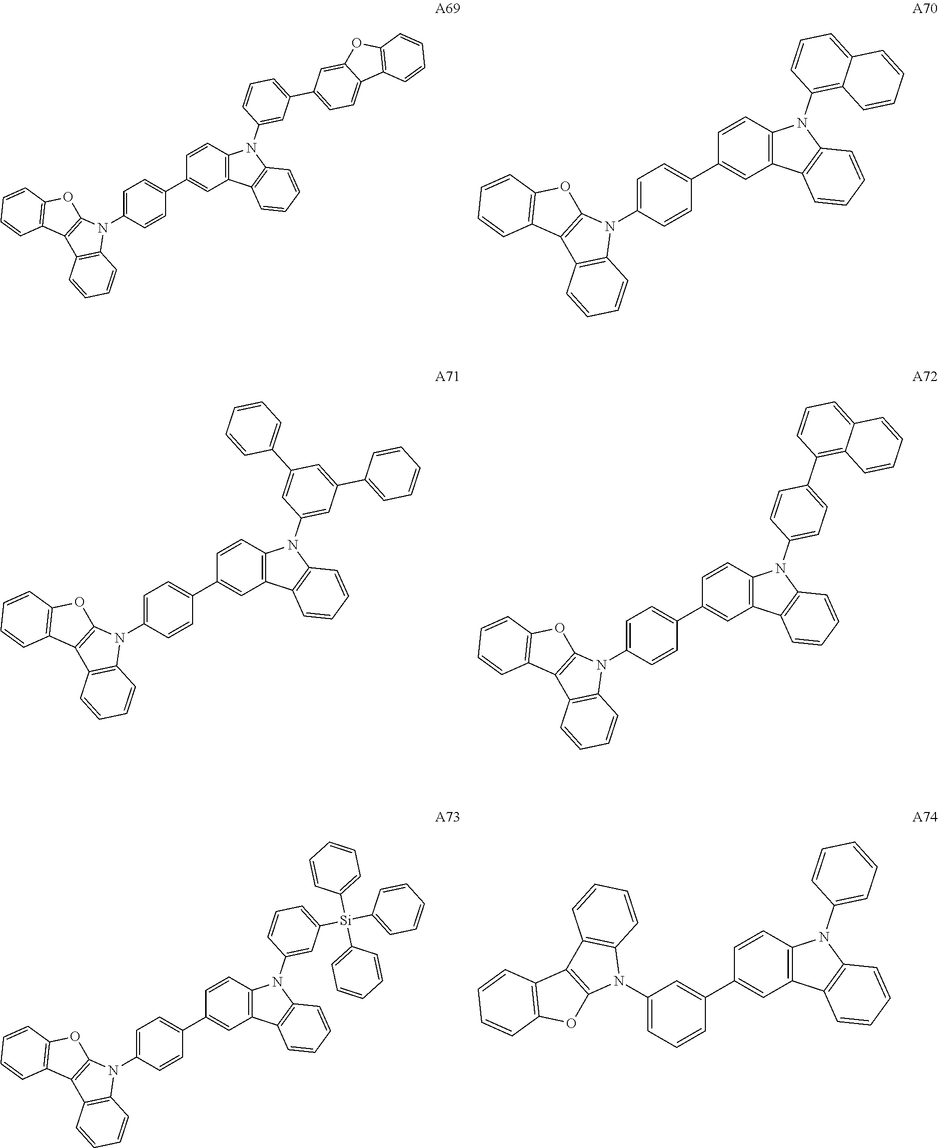

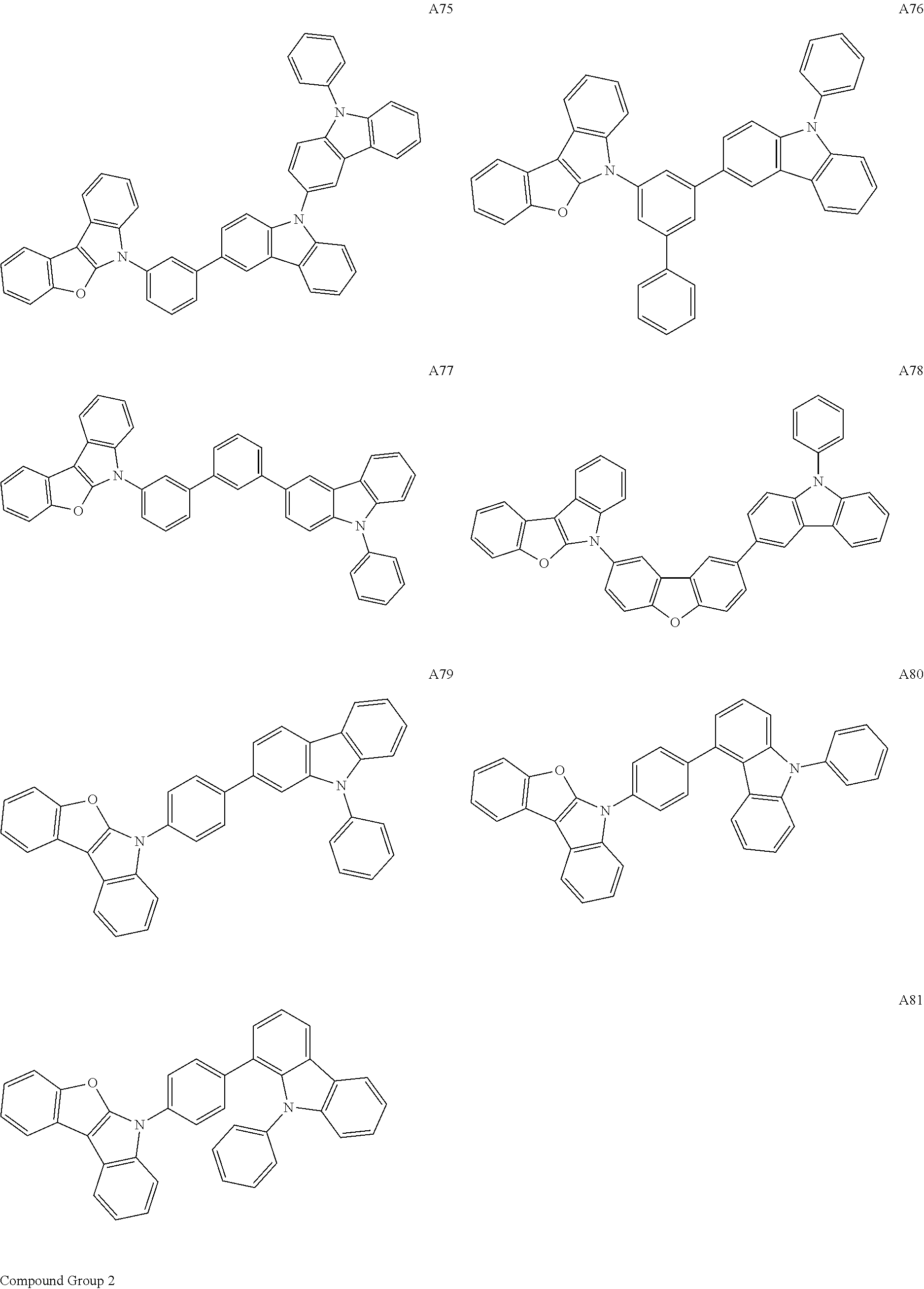

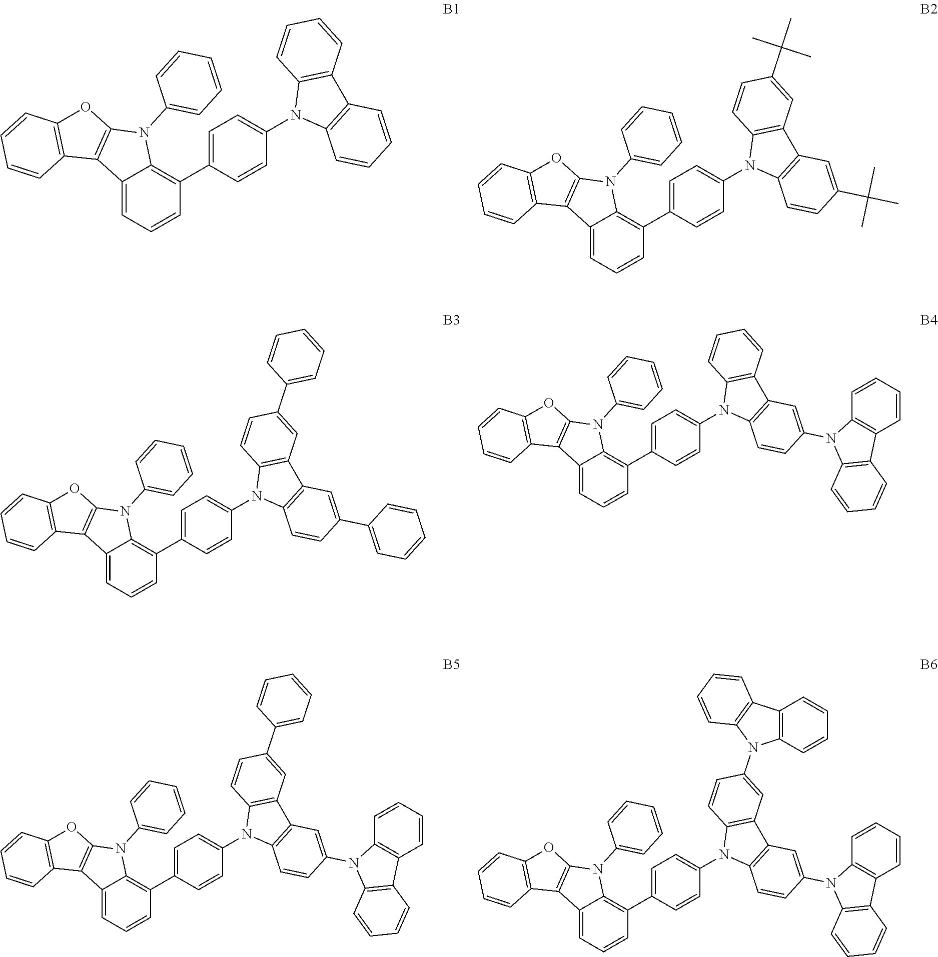

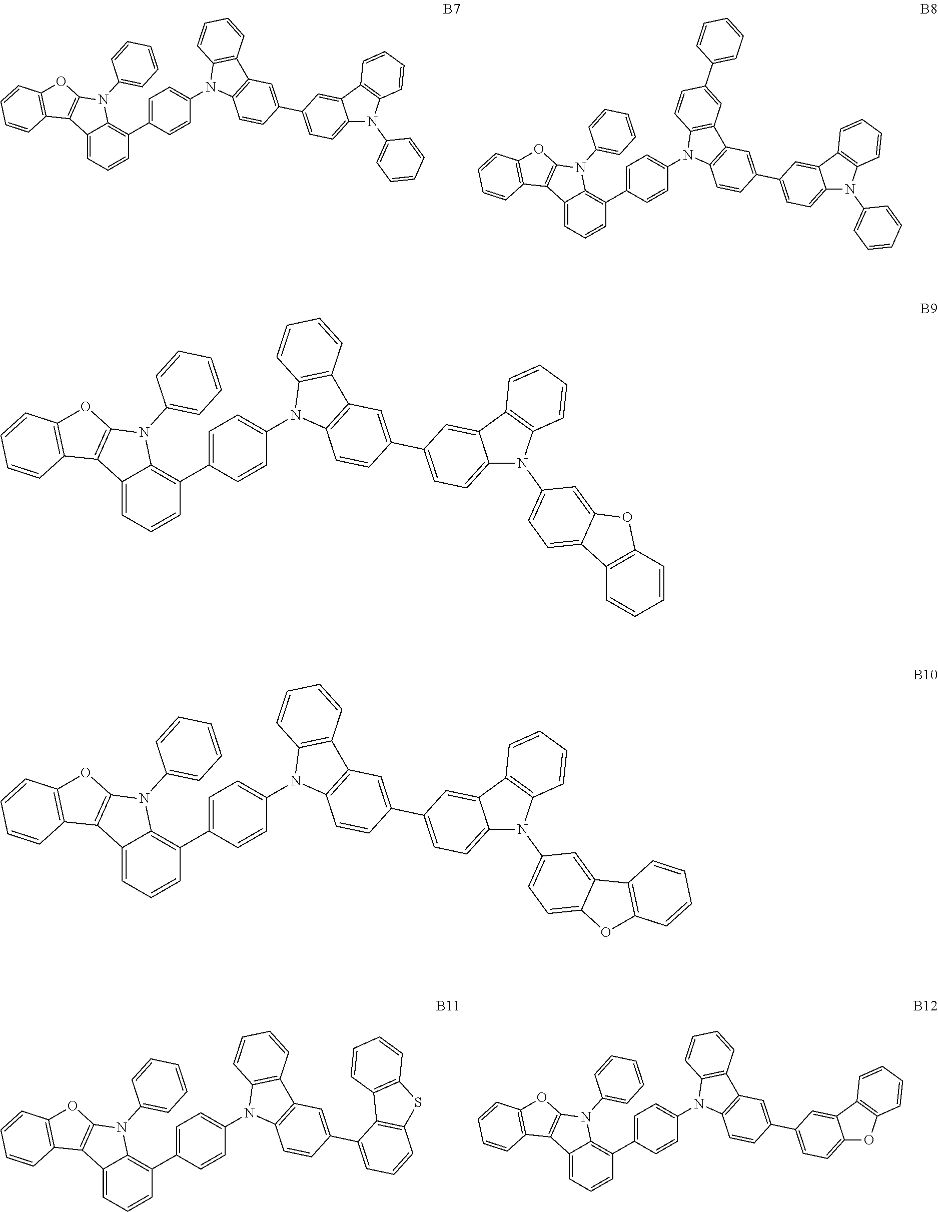

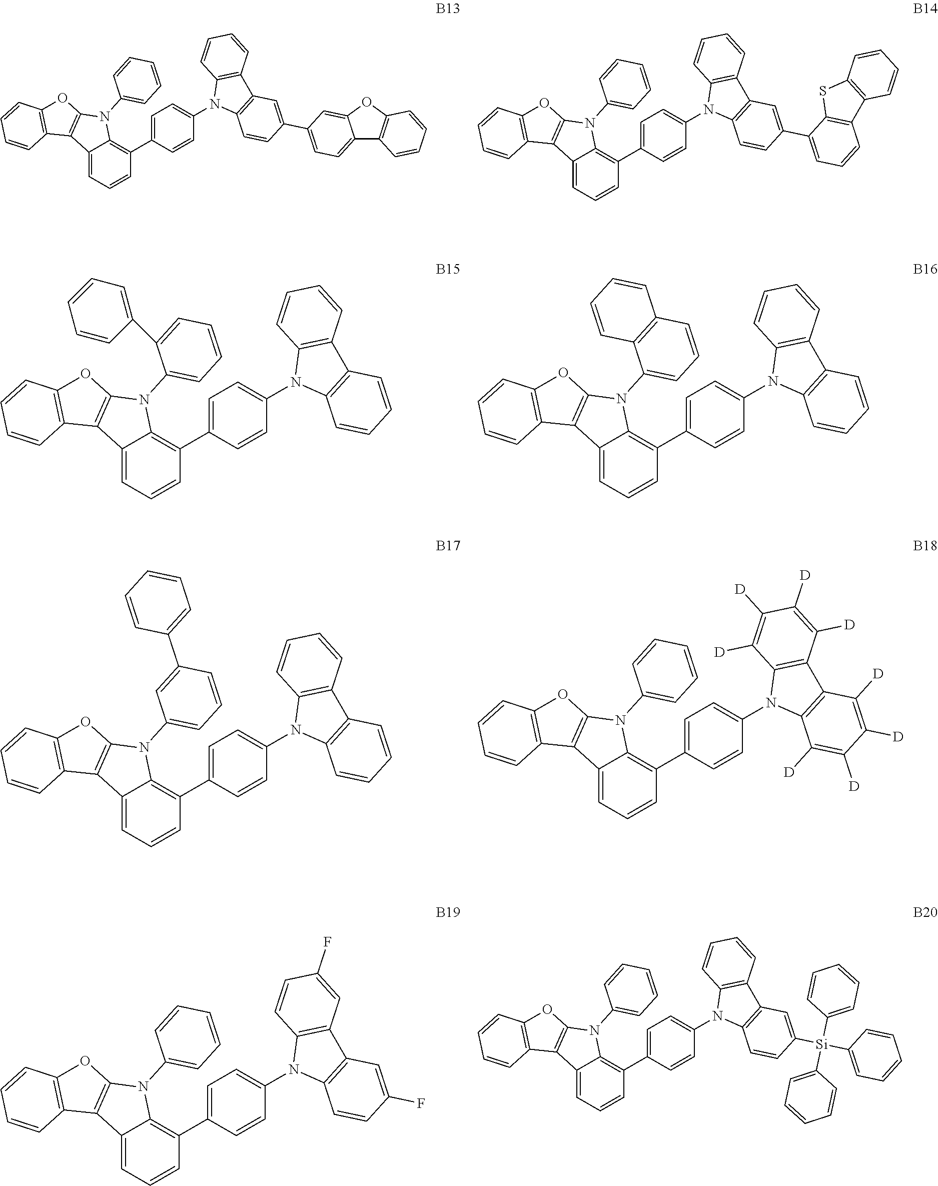

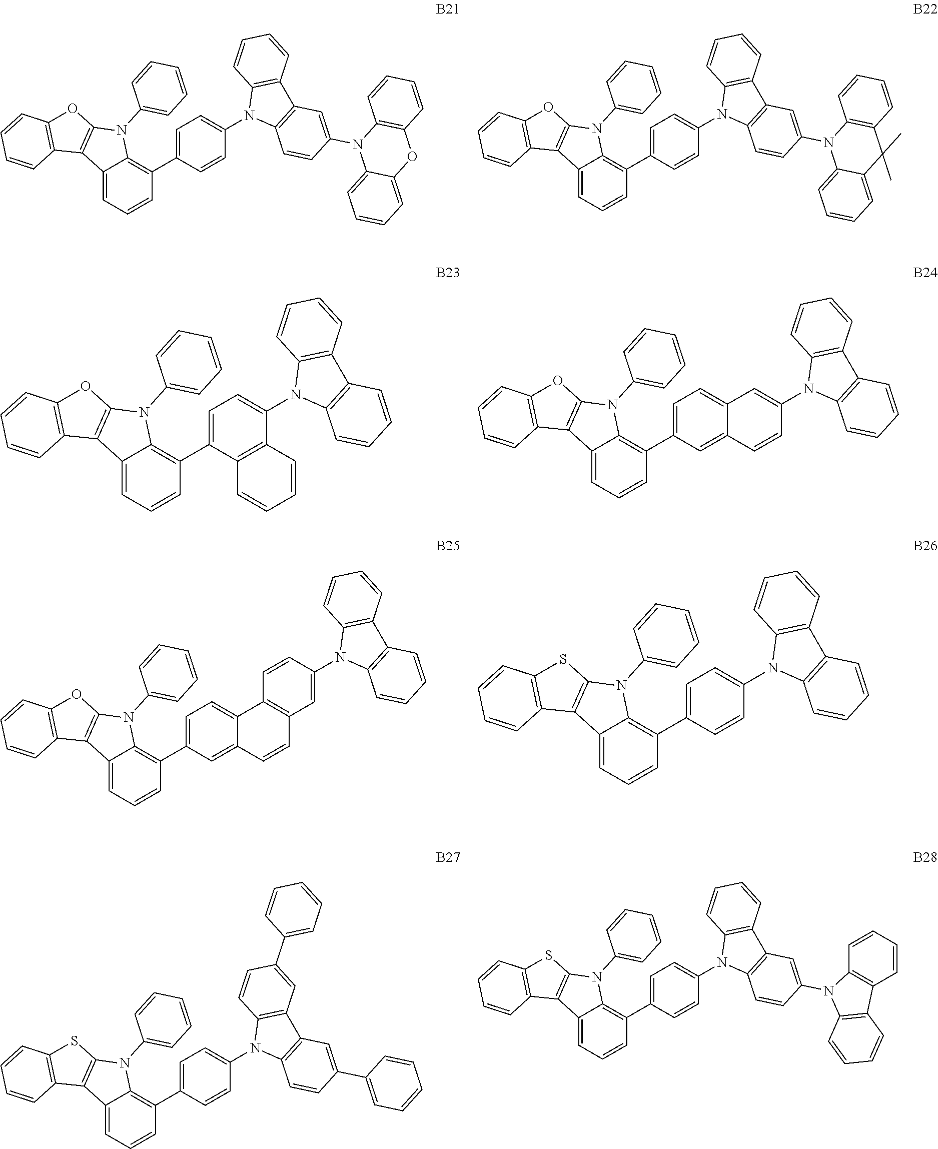

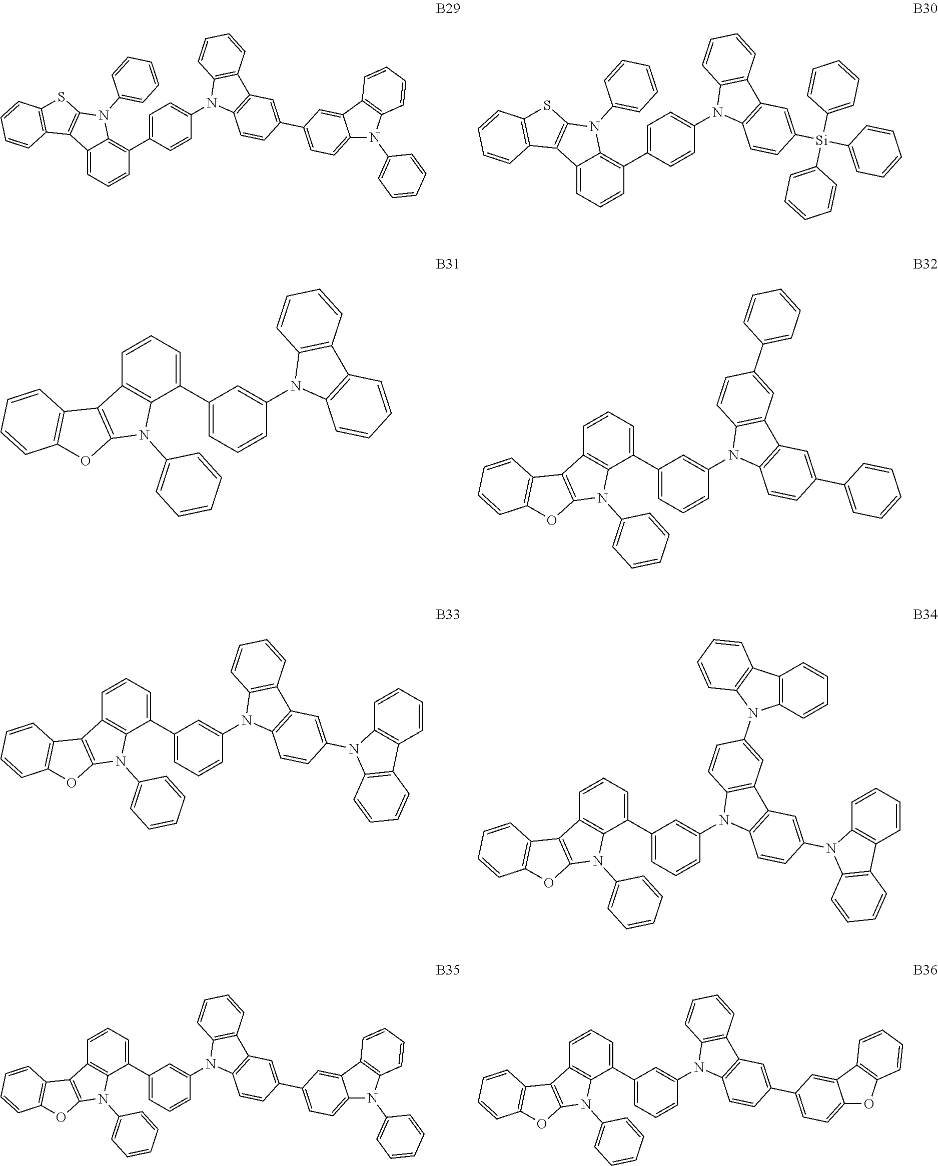

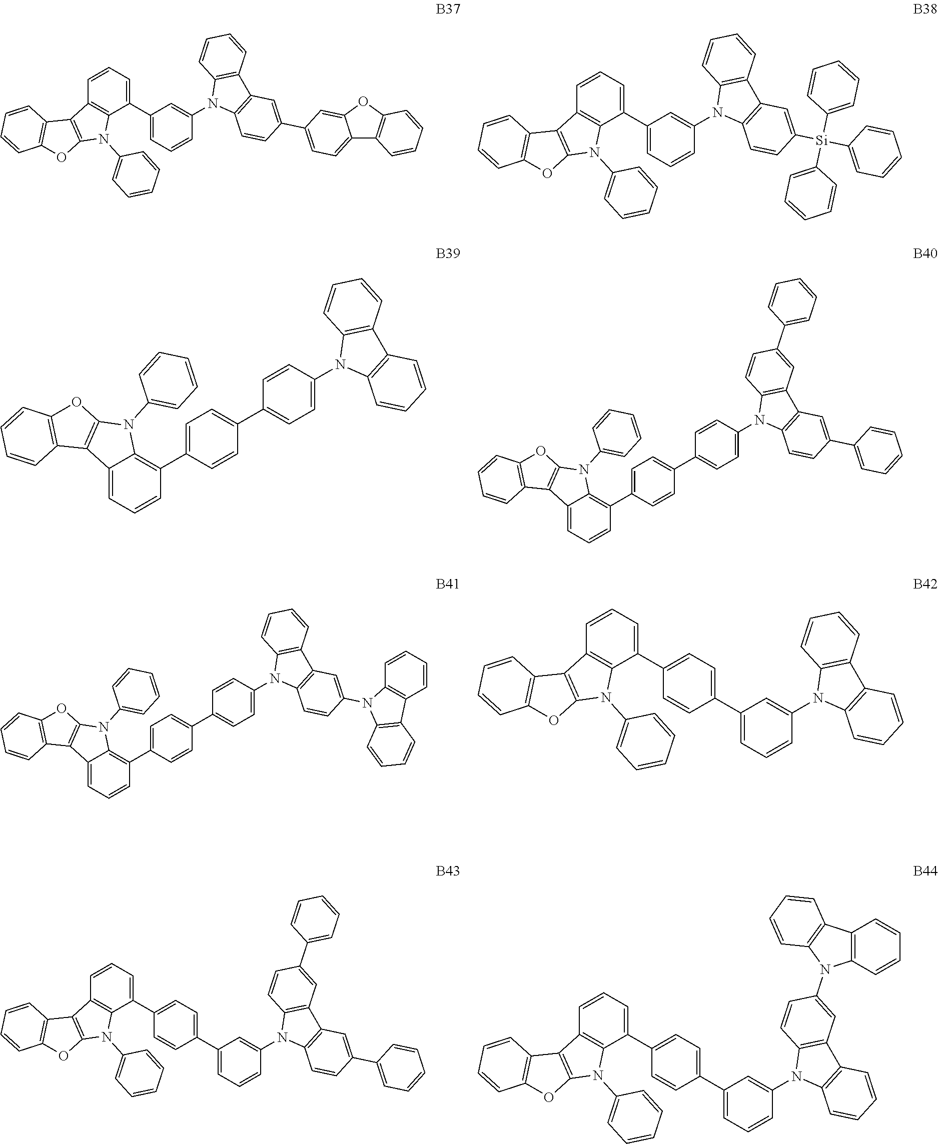

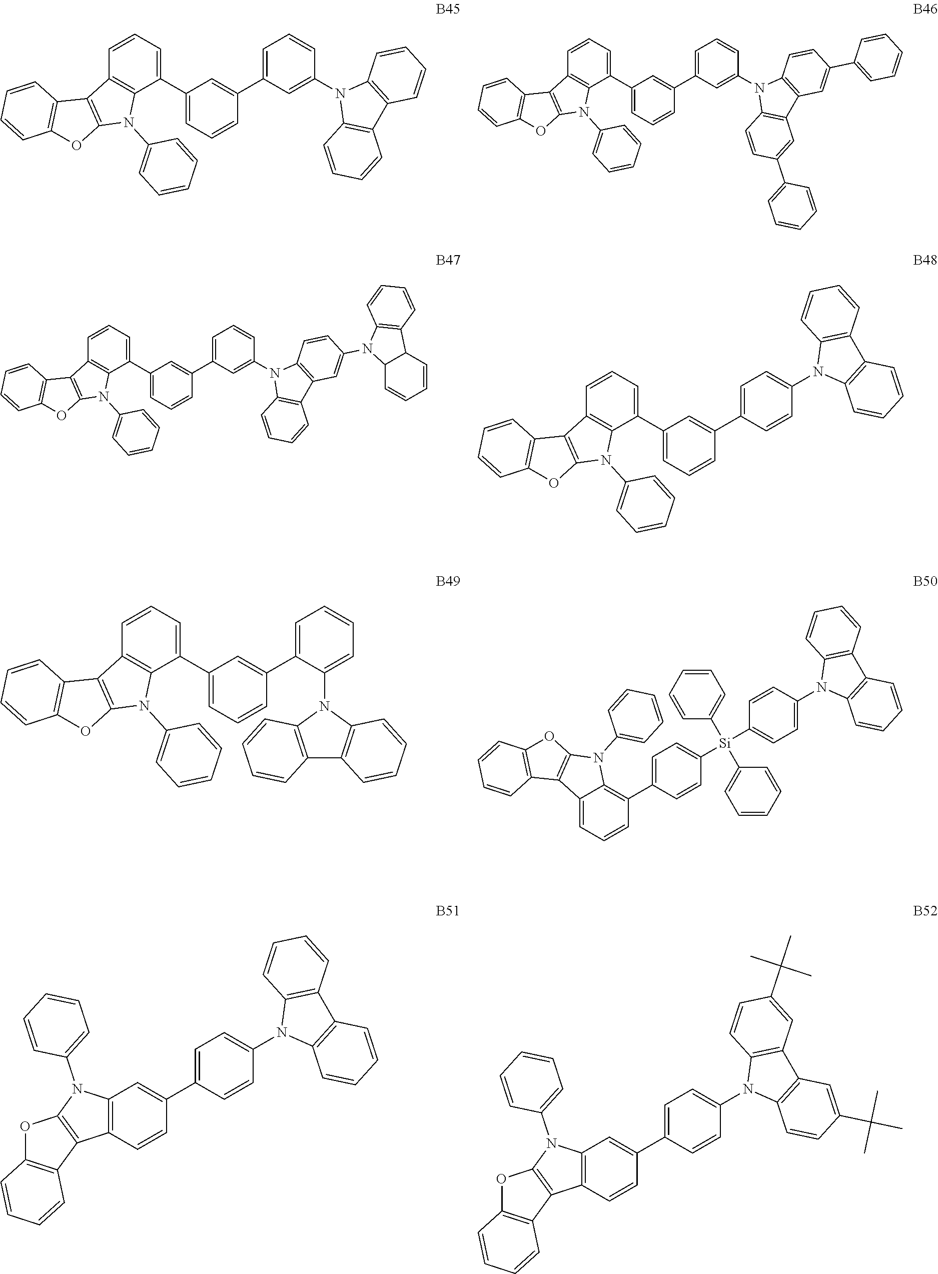

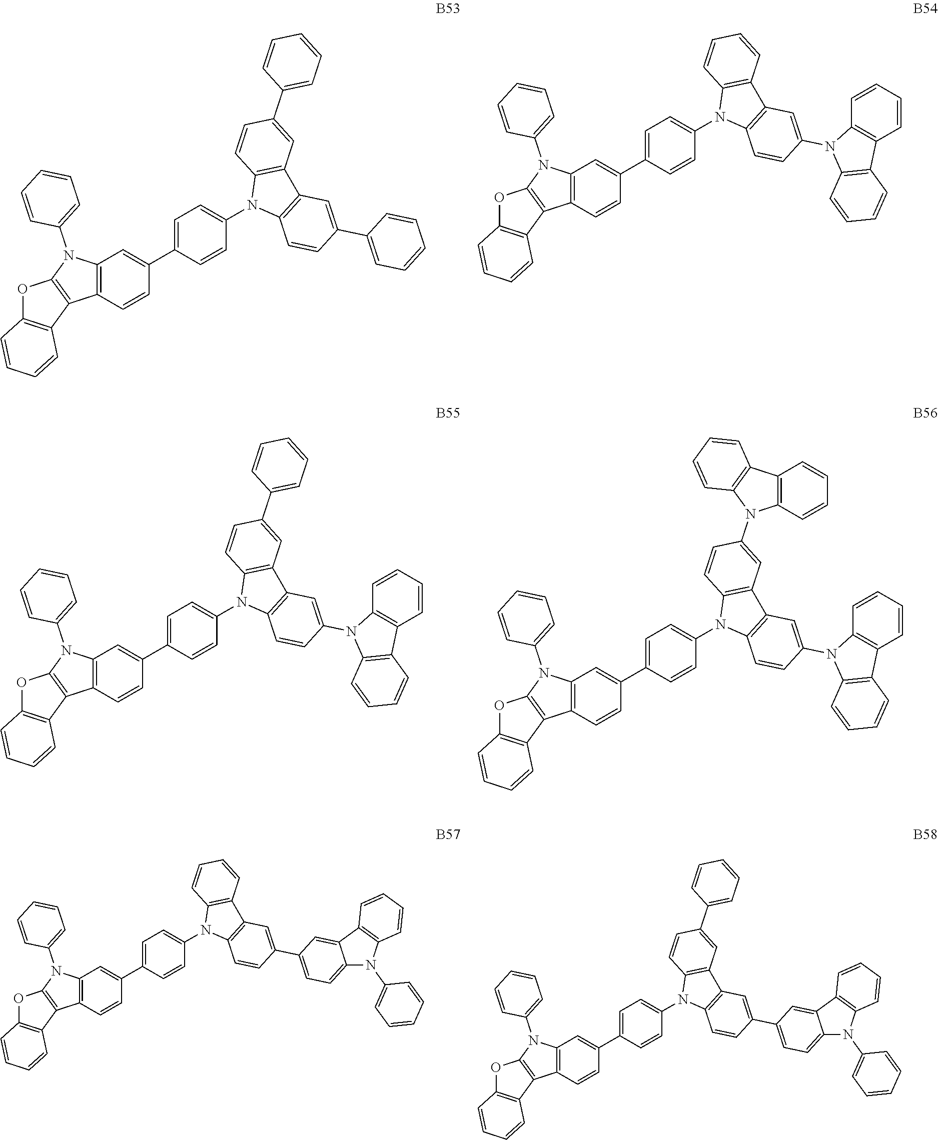

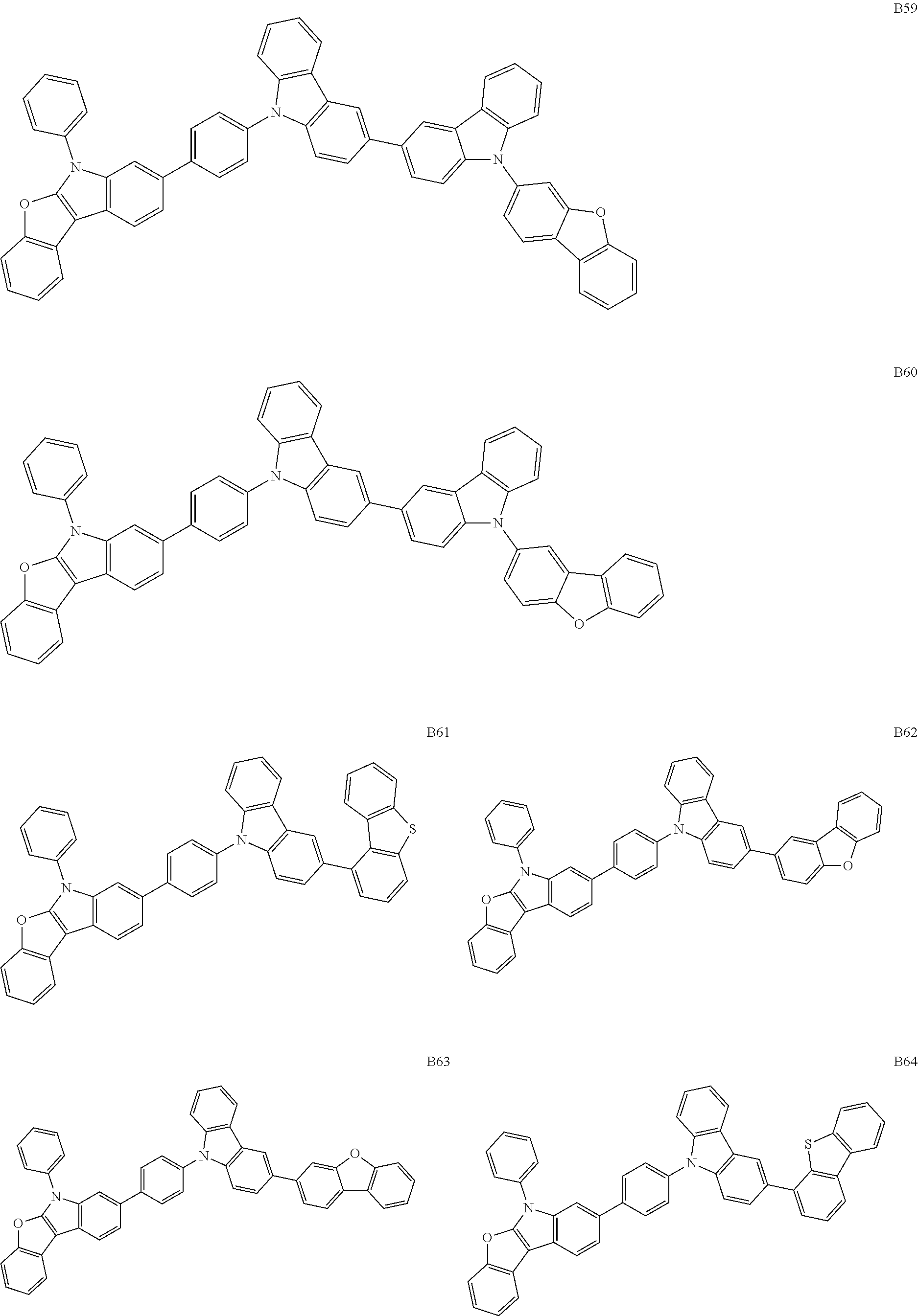

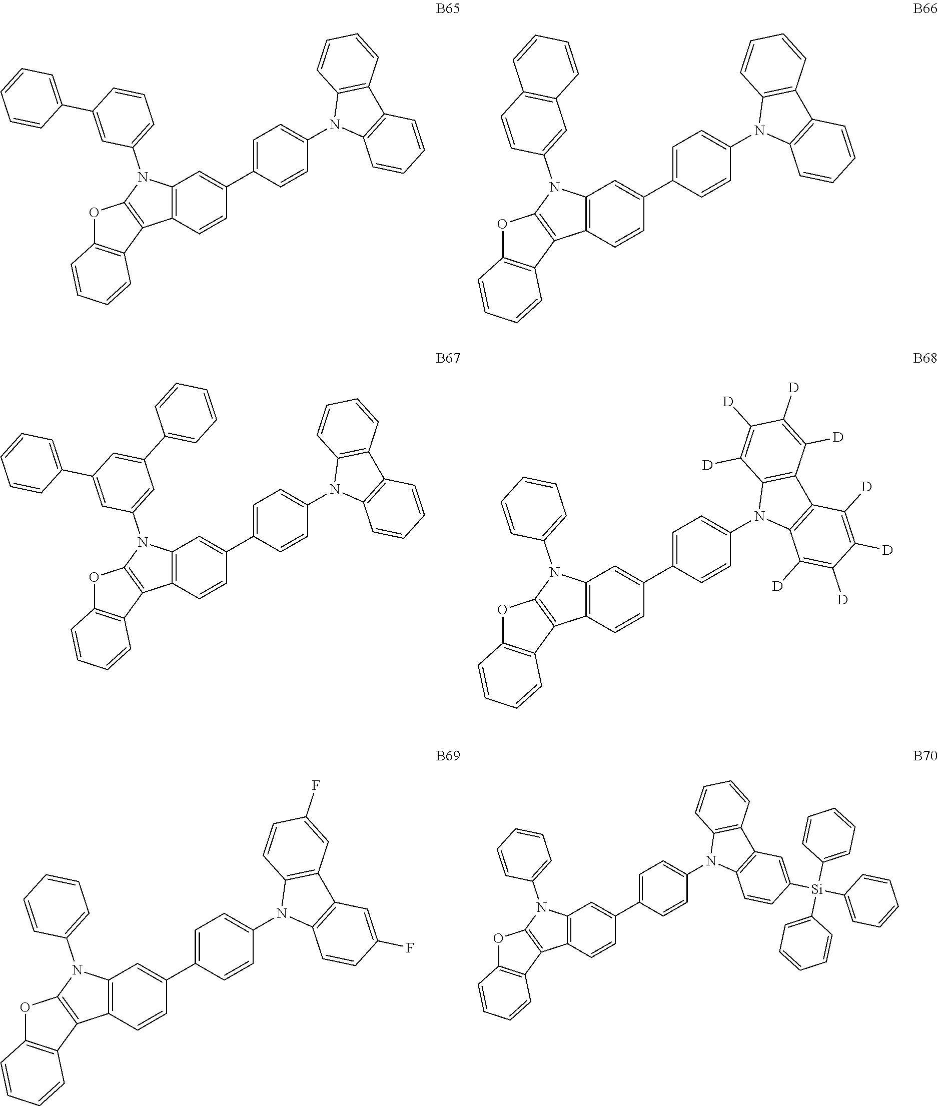

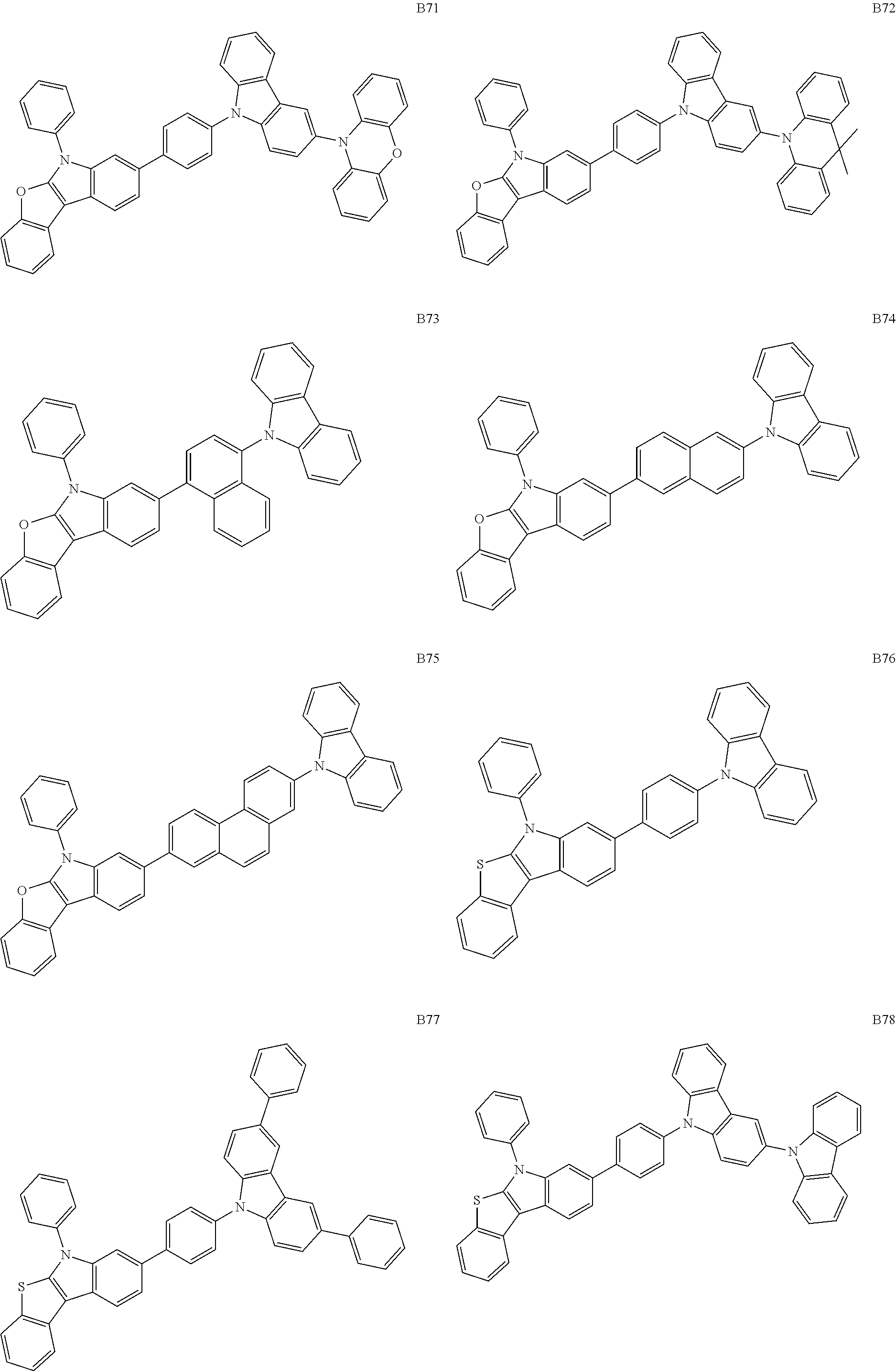

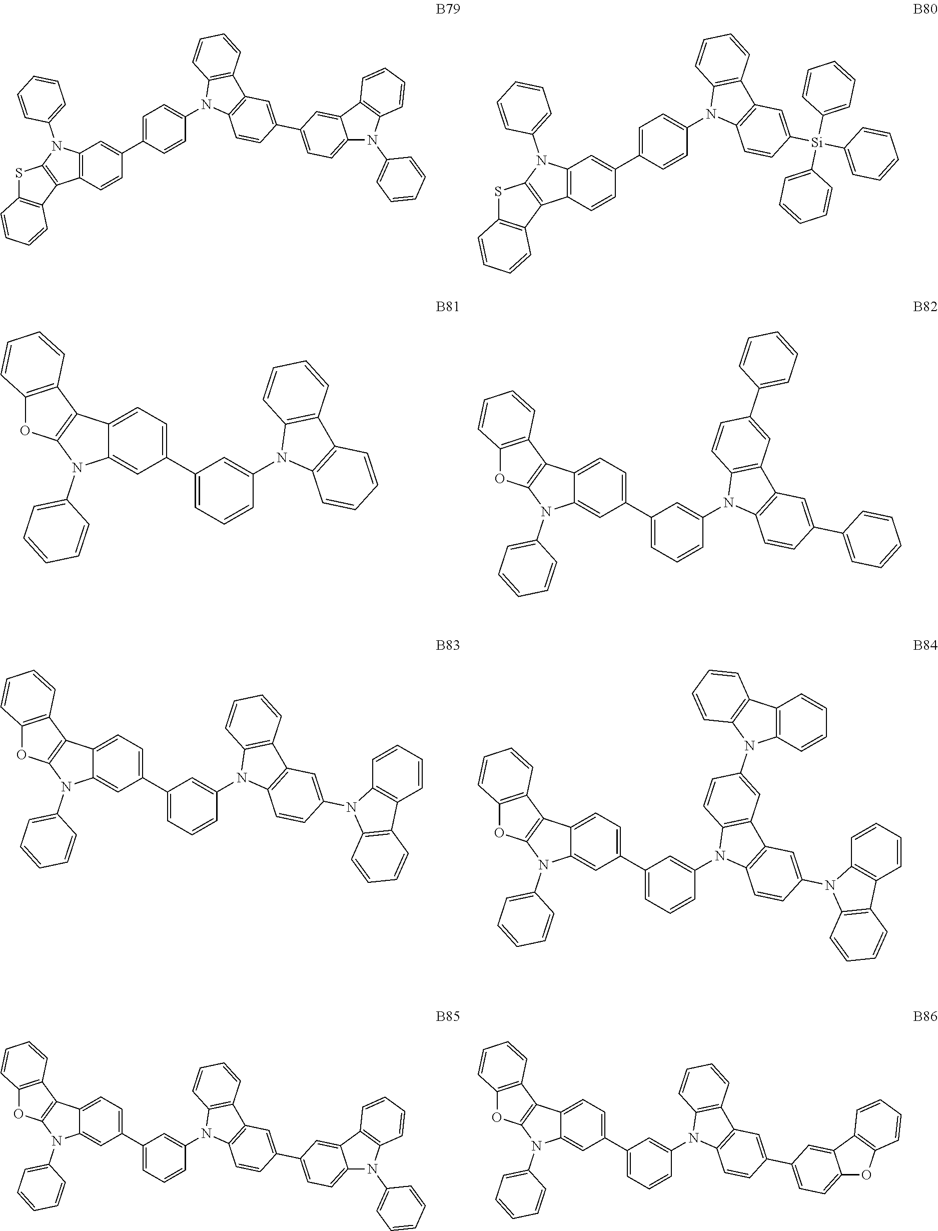

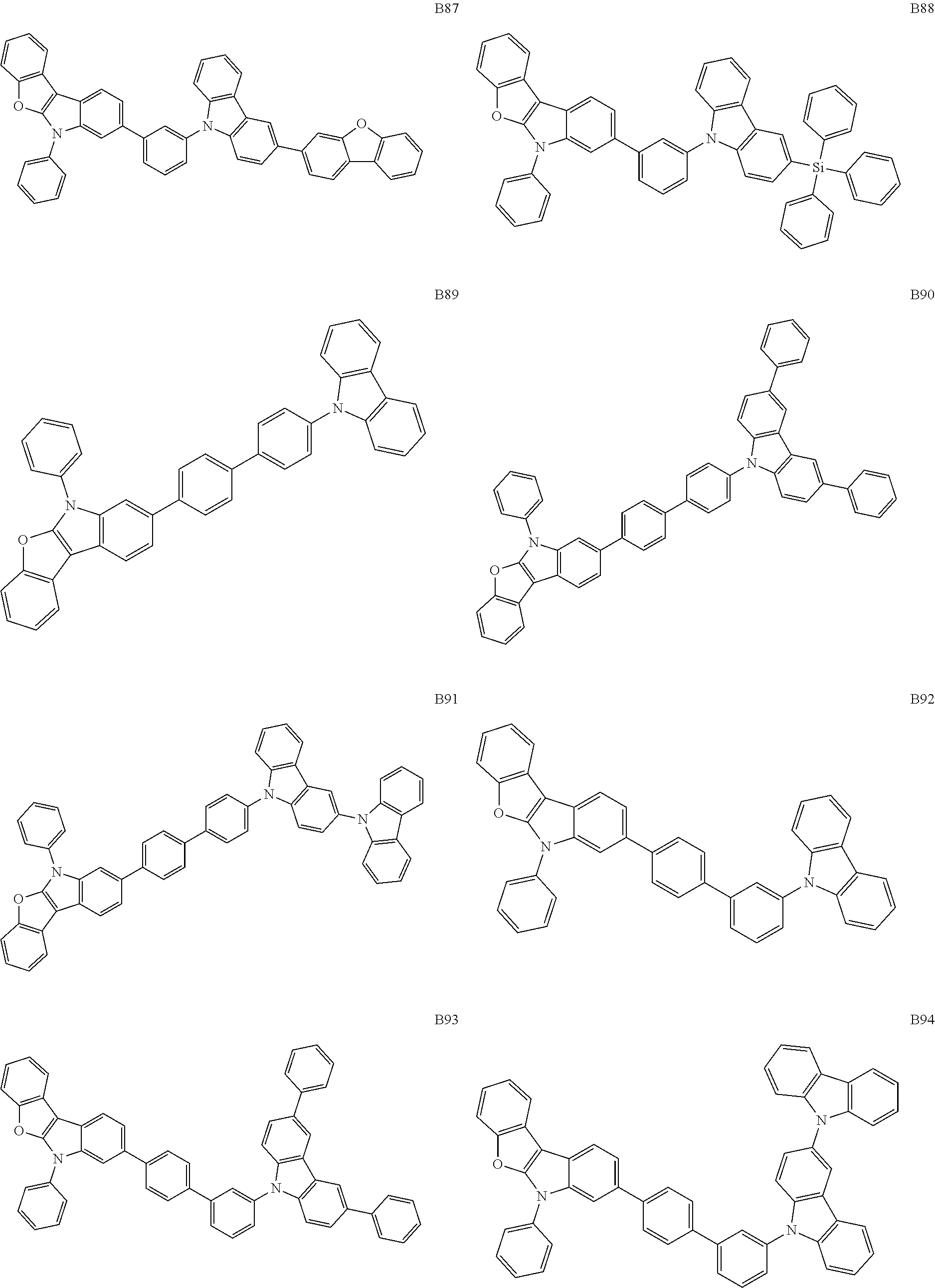

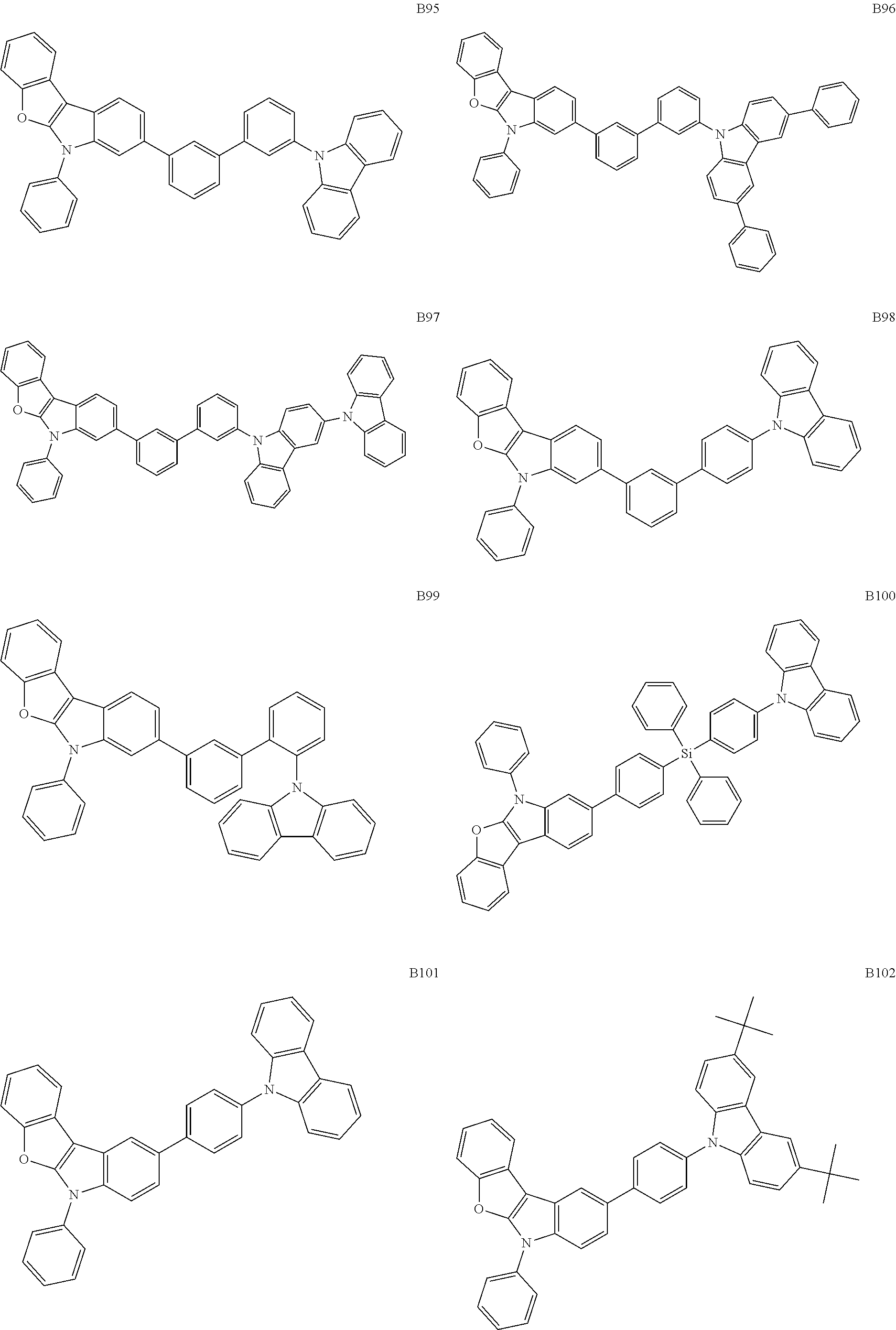

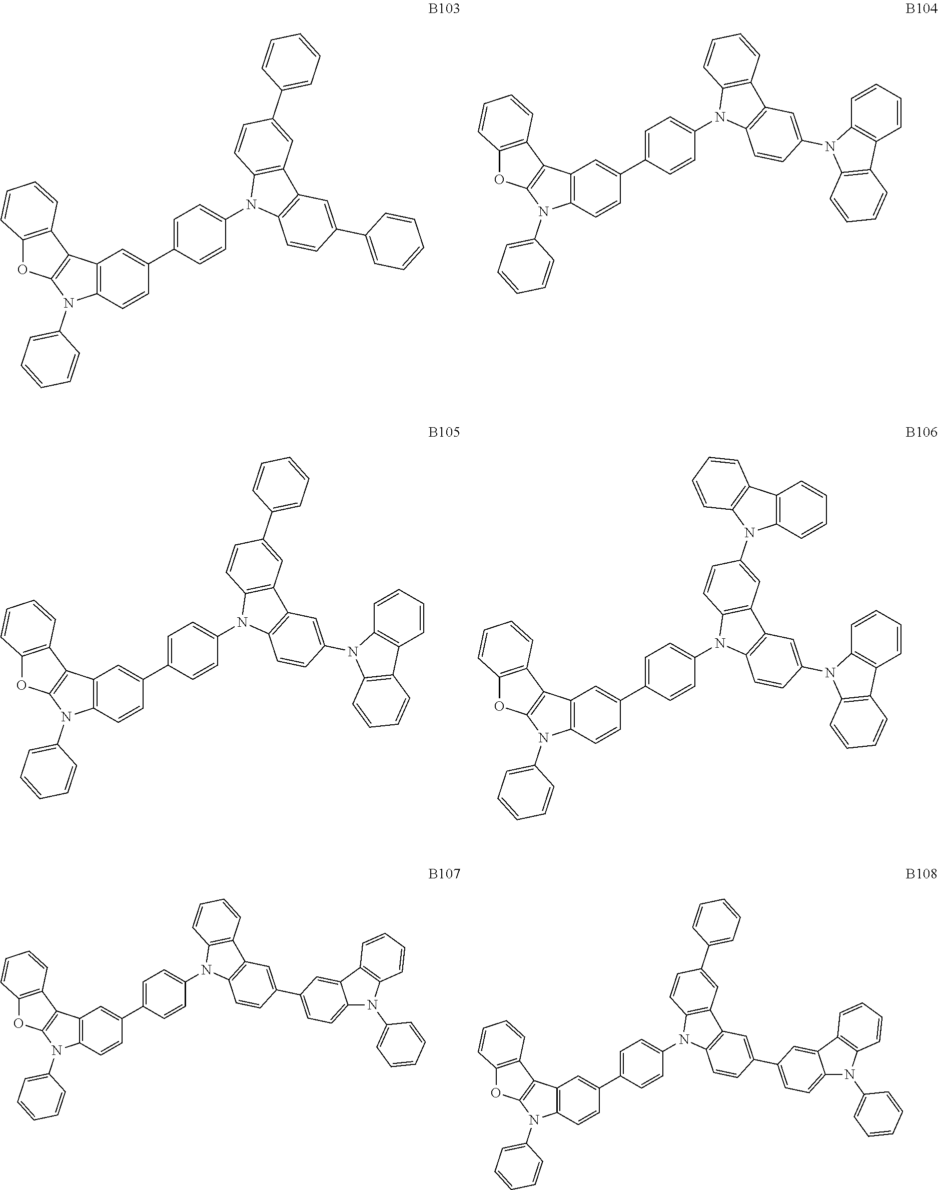

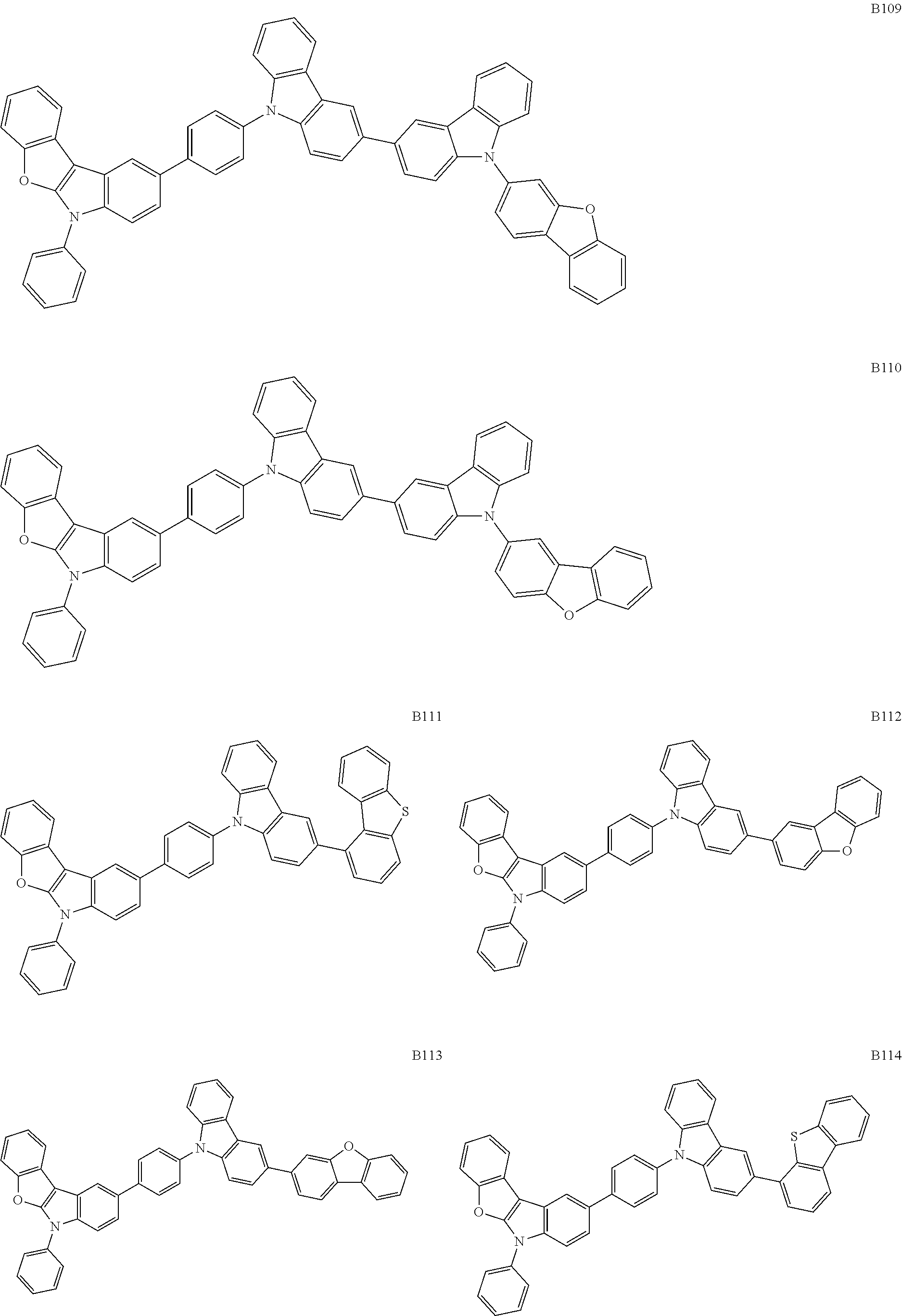

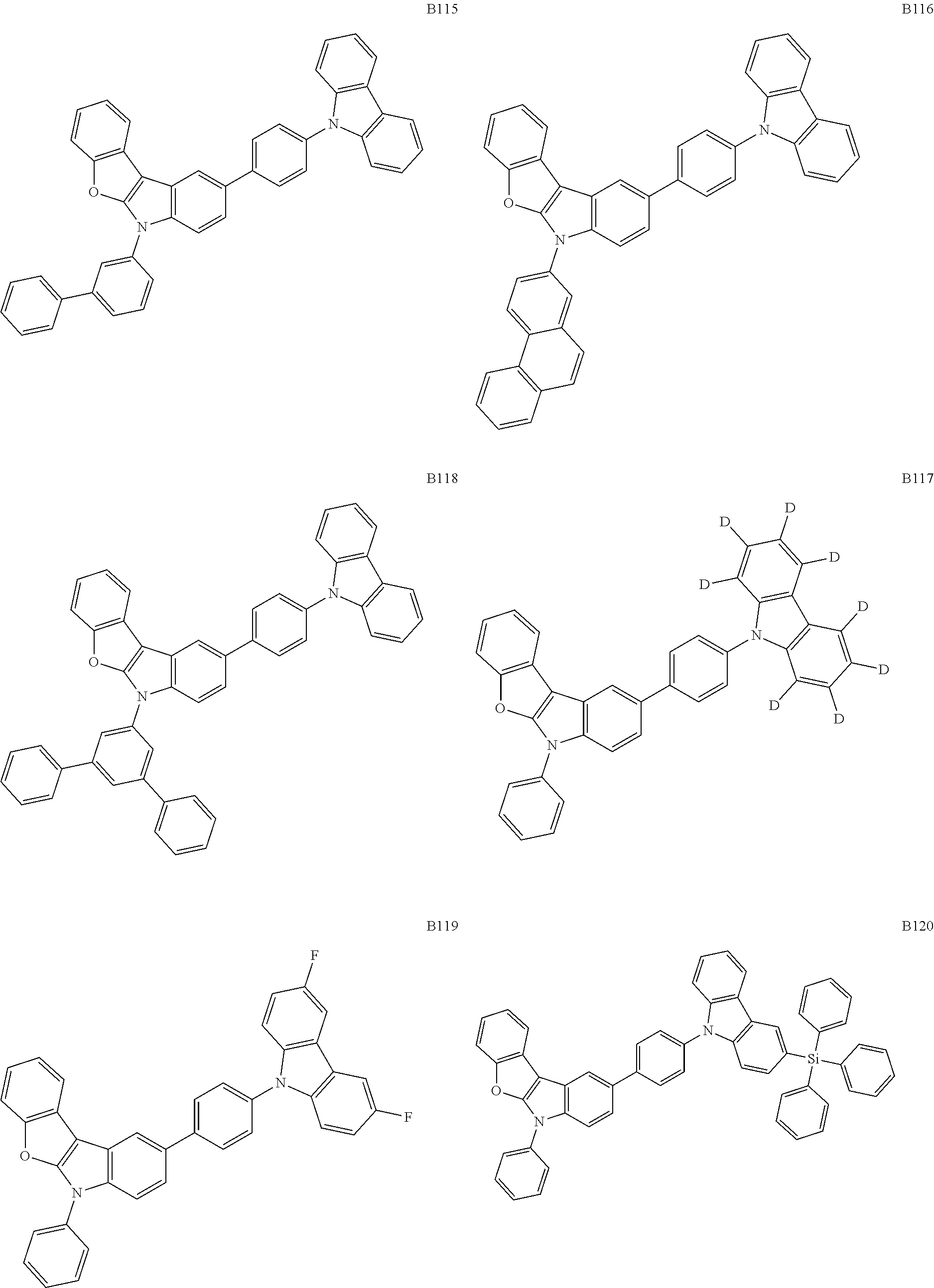

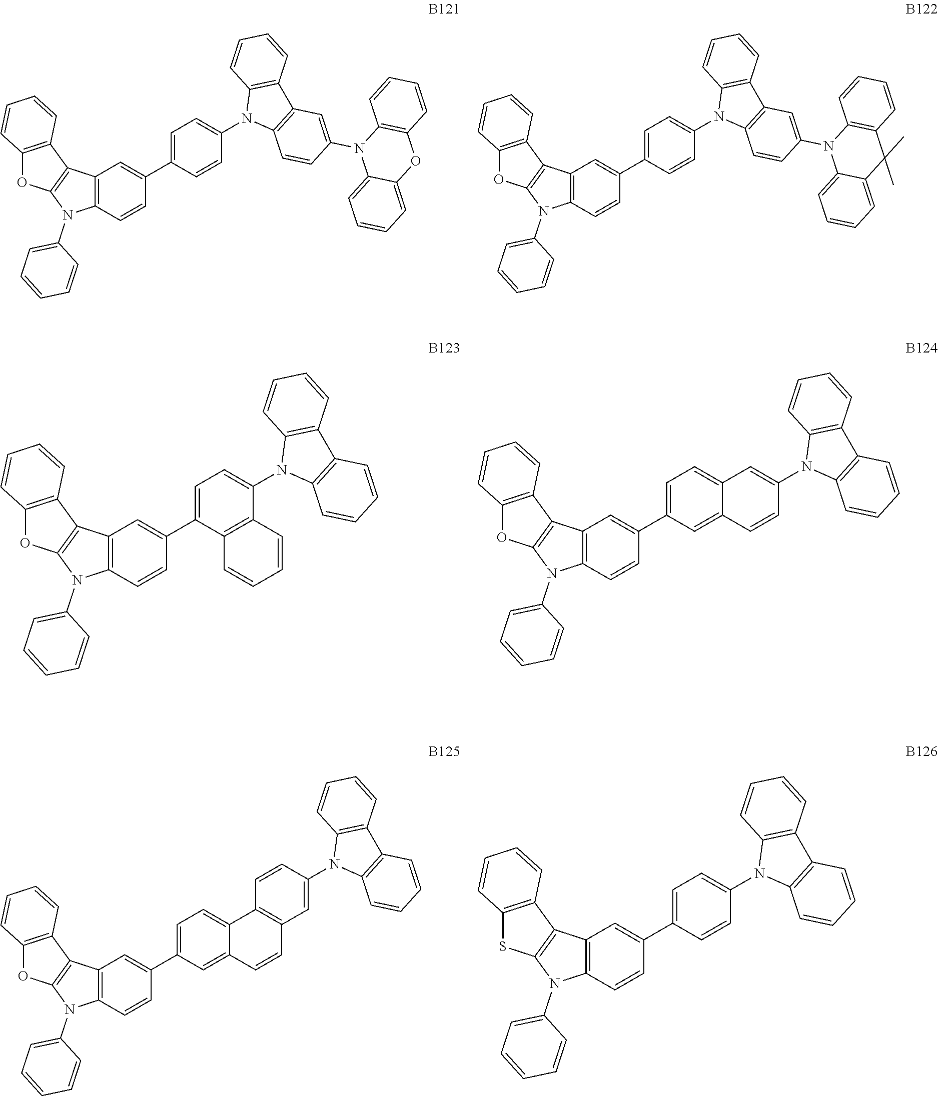 [SMILES: c1ccc(-n2c3ccc(-c4ccc(-n5c6ccccc6c6cc(N7c8ccccc8Oc8ccccc87)ccc65)cc4)cc3c3c4ccccc4oc32)cc1 ; CC1(C)c2ccccc2N(c2ccc3c(c2)c2ccccc2n3-c2ccc(-c3ccc4c(c3)c3c5ccccc5oc3n4-c3ccccc3)cc2)c2ccccc21 ; c1ccc(-n2c3ccc(-c4ccc(-n5c6ccccc6c6ccccc65)c5ccccc45)cc3c3c4ccccc4oc32)cc1 ; c1ccc(-n2c3ccc(-c4ccc5cc(-n6c7ccccc7c7ccccc76)ccc5c4)cc3c3c4ccccc4oc32)cc1 ; c1ccc(-n2c3ccc(-c4ccc5c(ccc6cc(-n7c8ccccc8c8ccccc87)ccc65)c4)cc3c3c4ccccc4oc32)cc1 ; c1ccc(-n2c3ccc(-c4ccc(-n5c6ccccc6c6ccccc65)cc4)cc3c3c4ccccc4sc32)cc1]

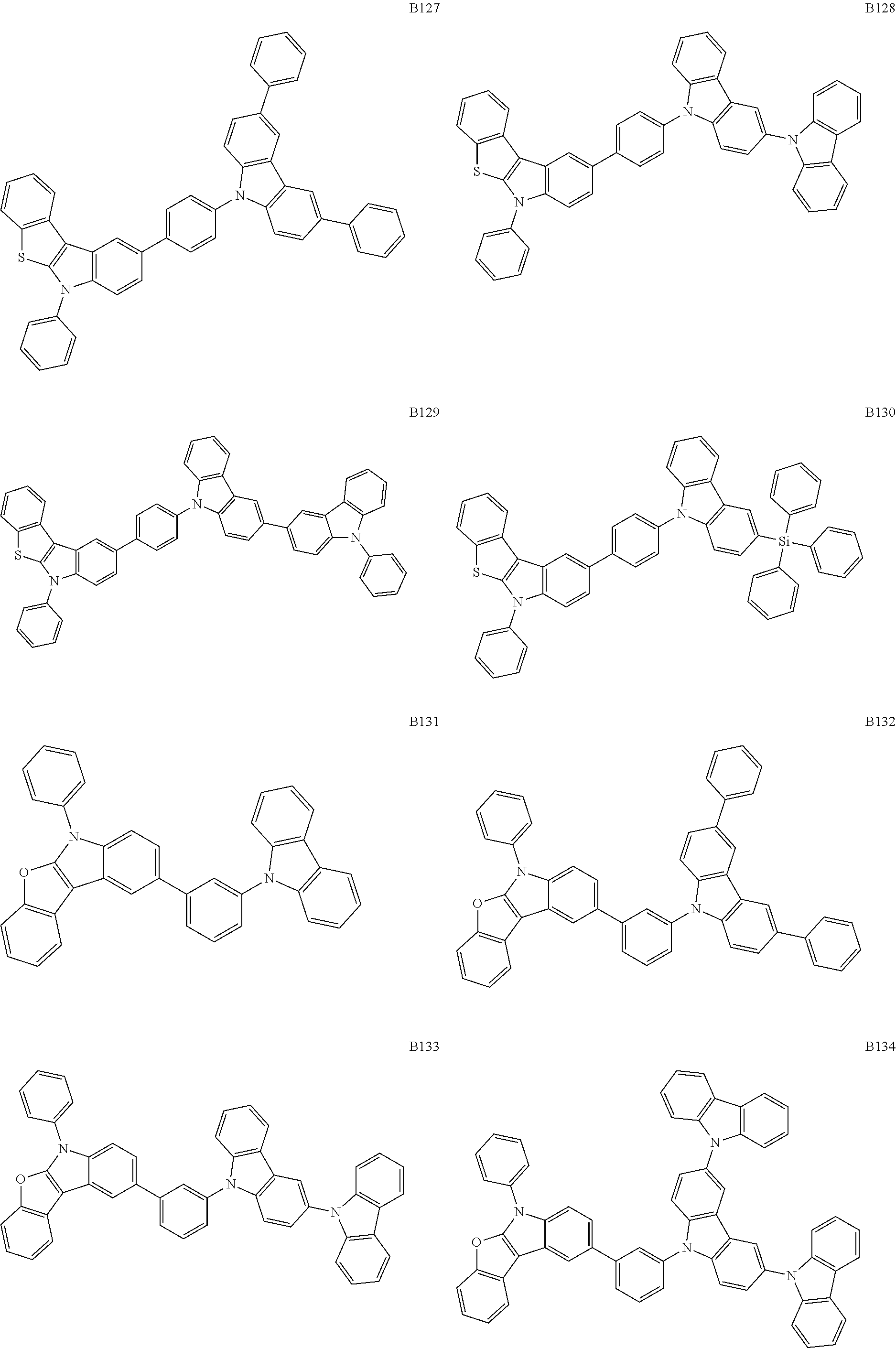

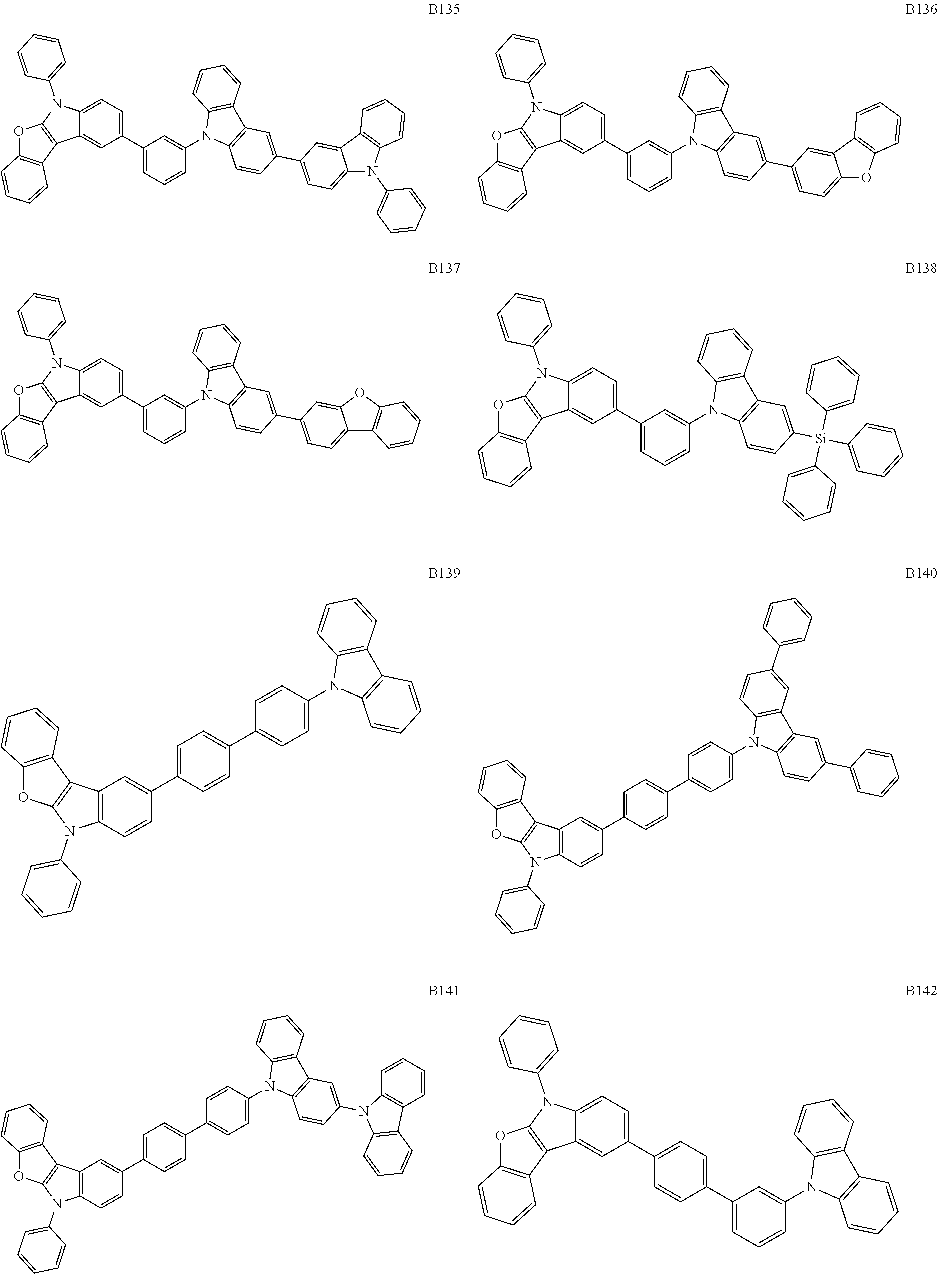

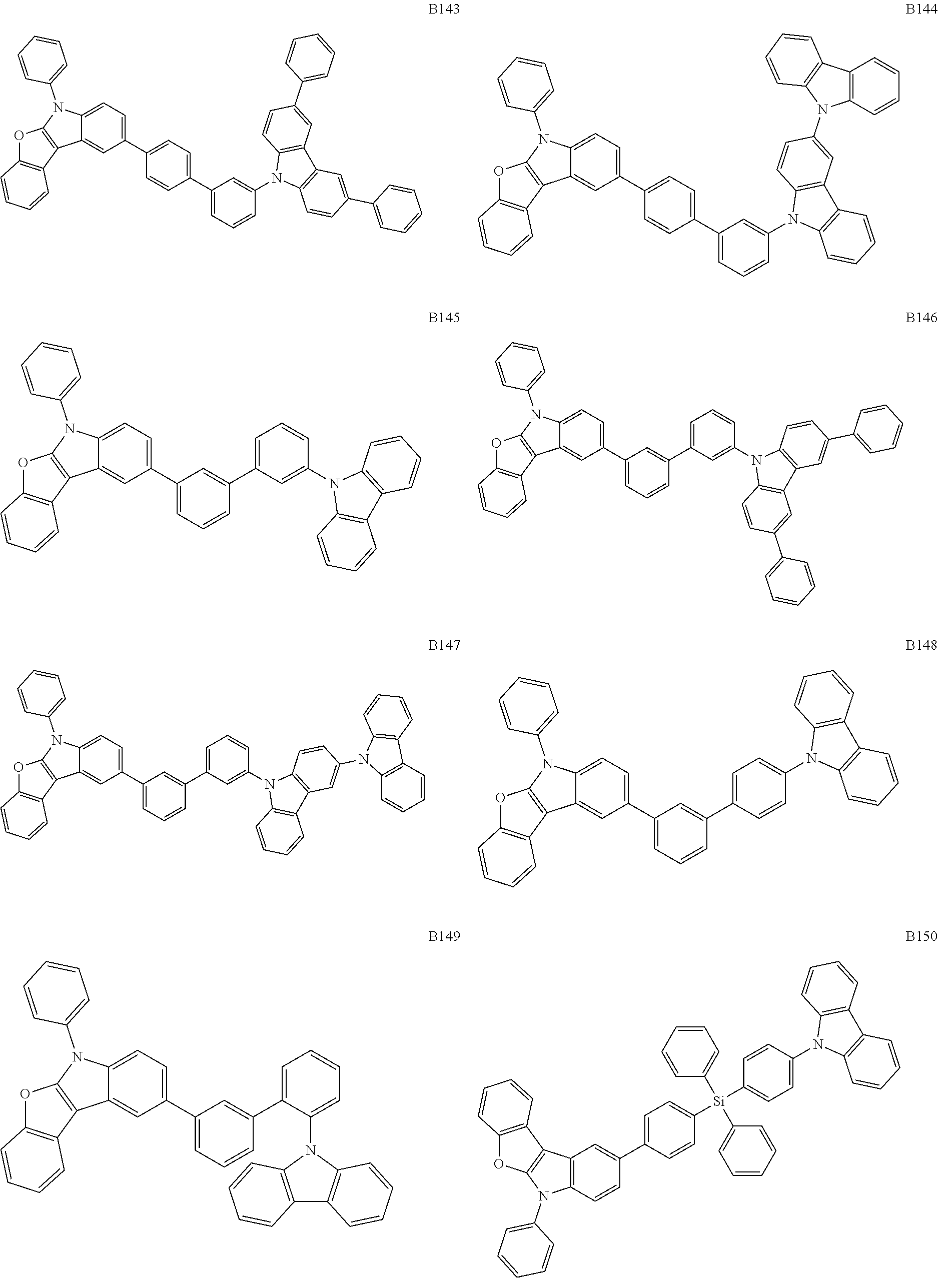

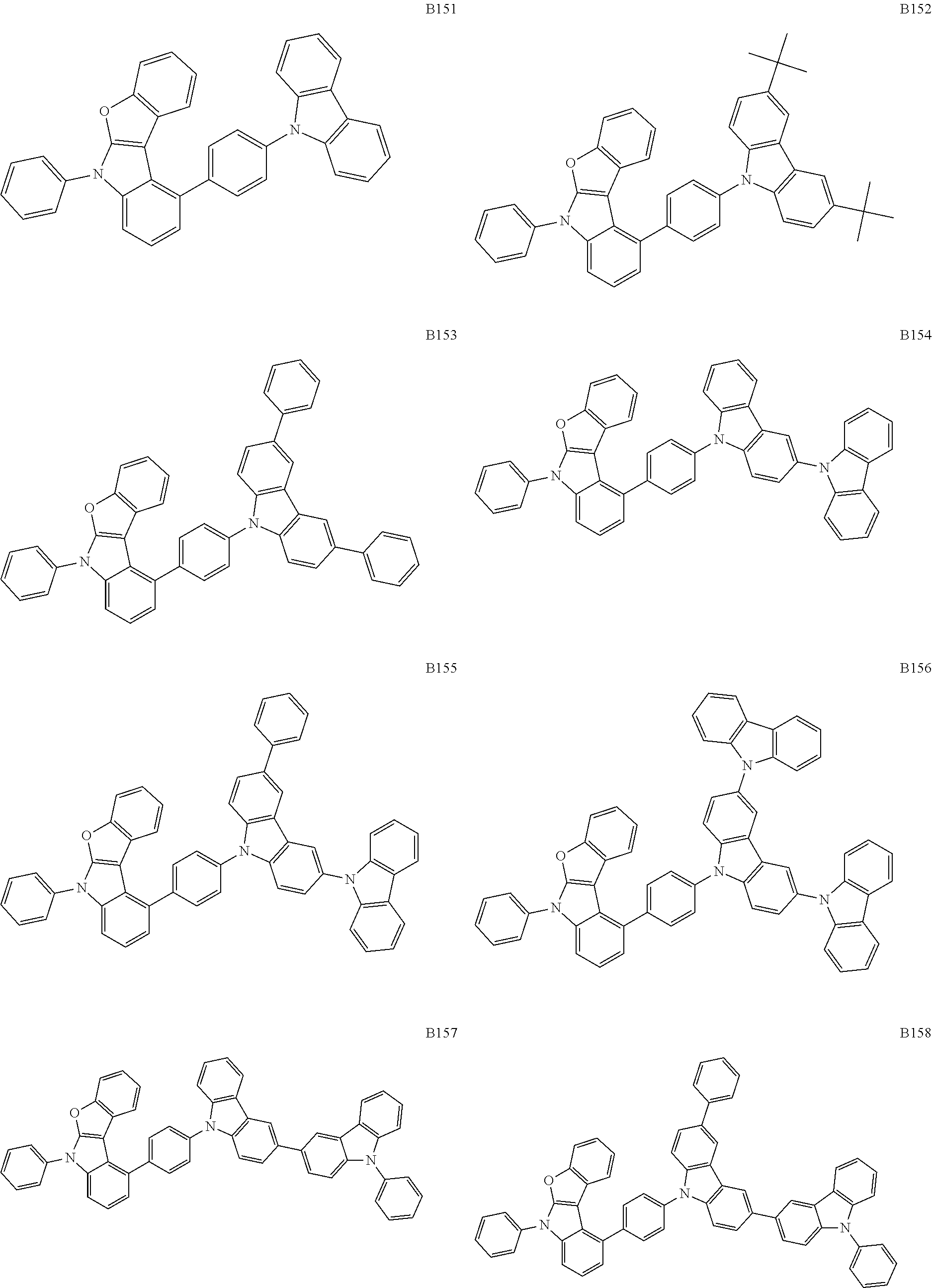

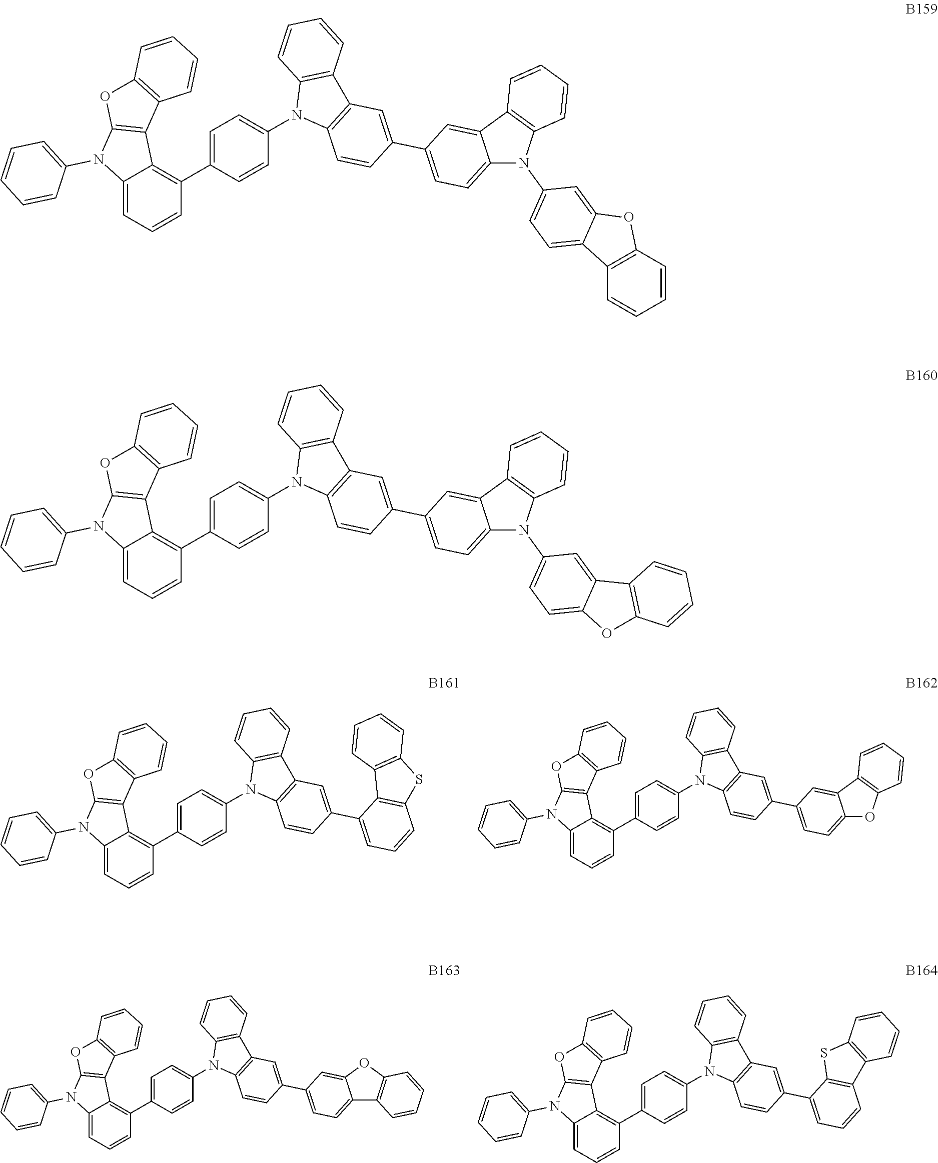

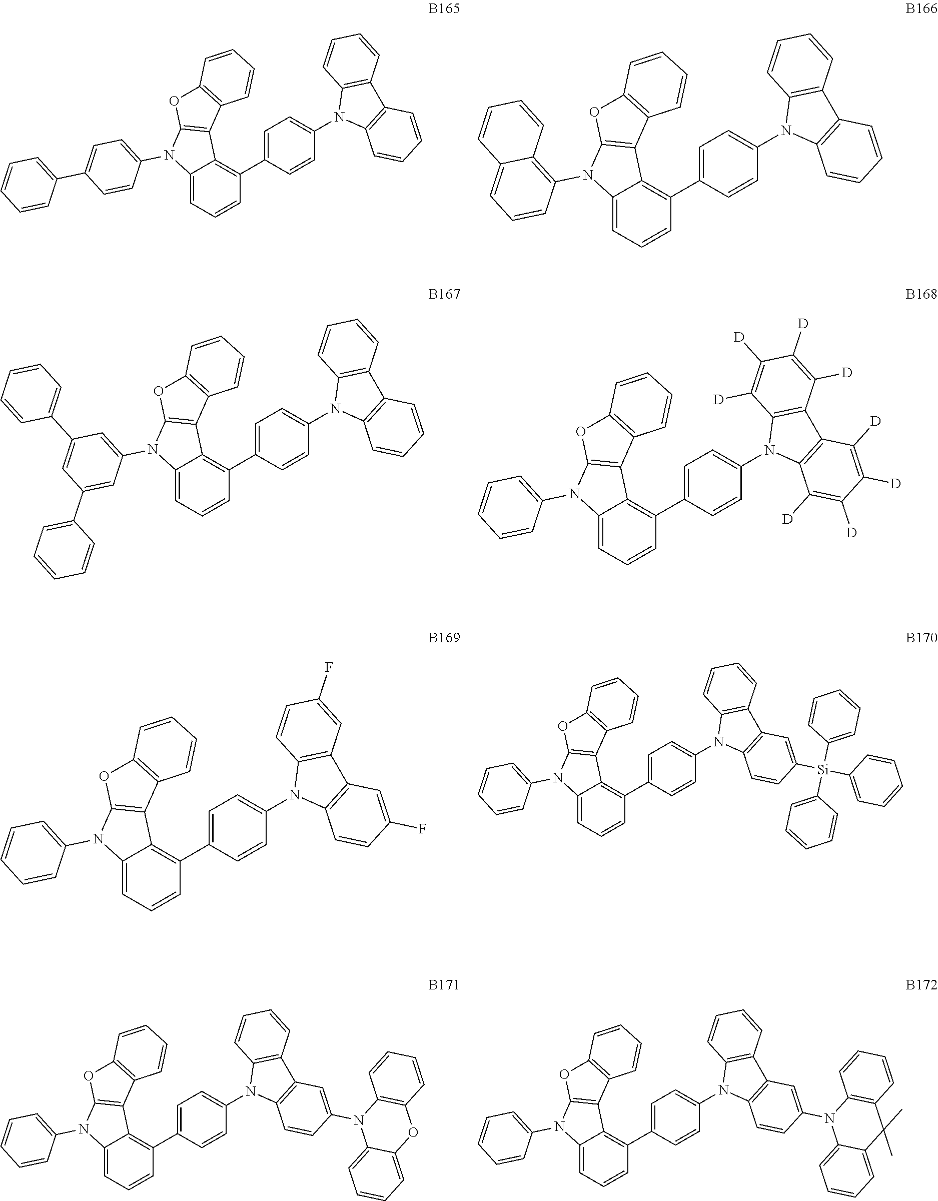

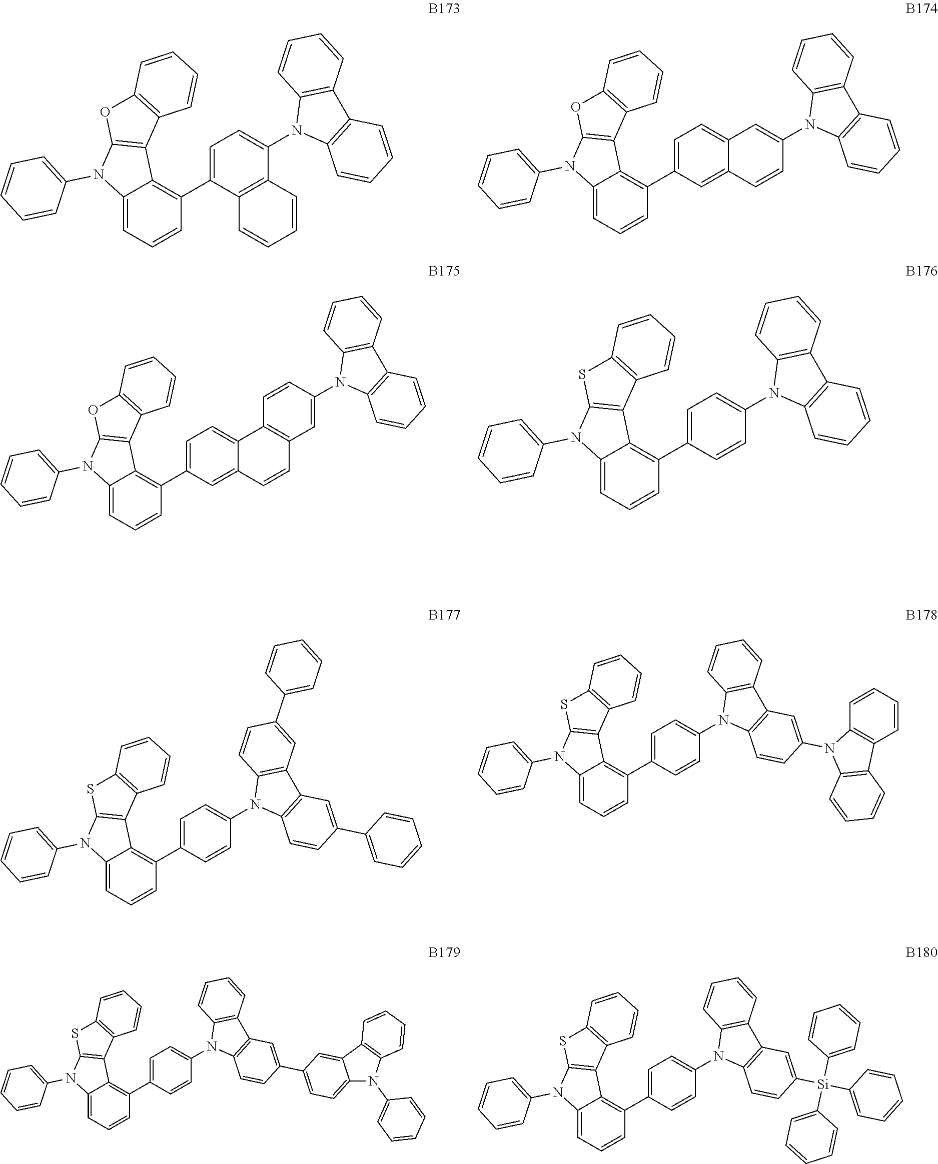

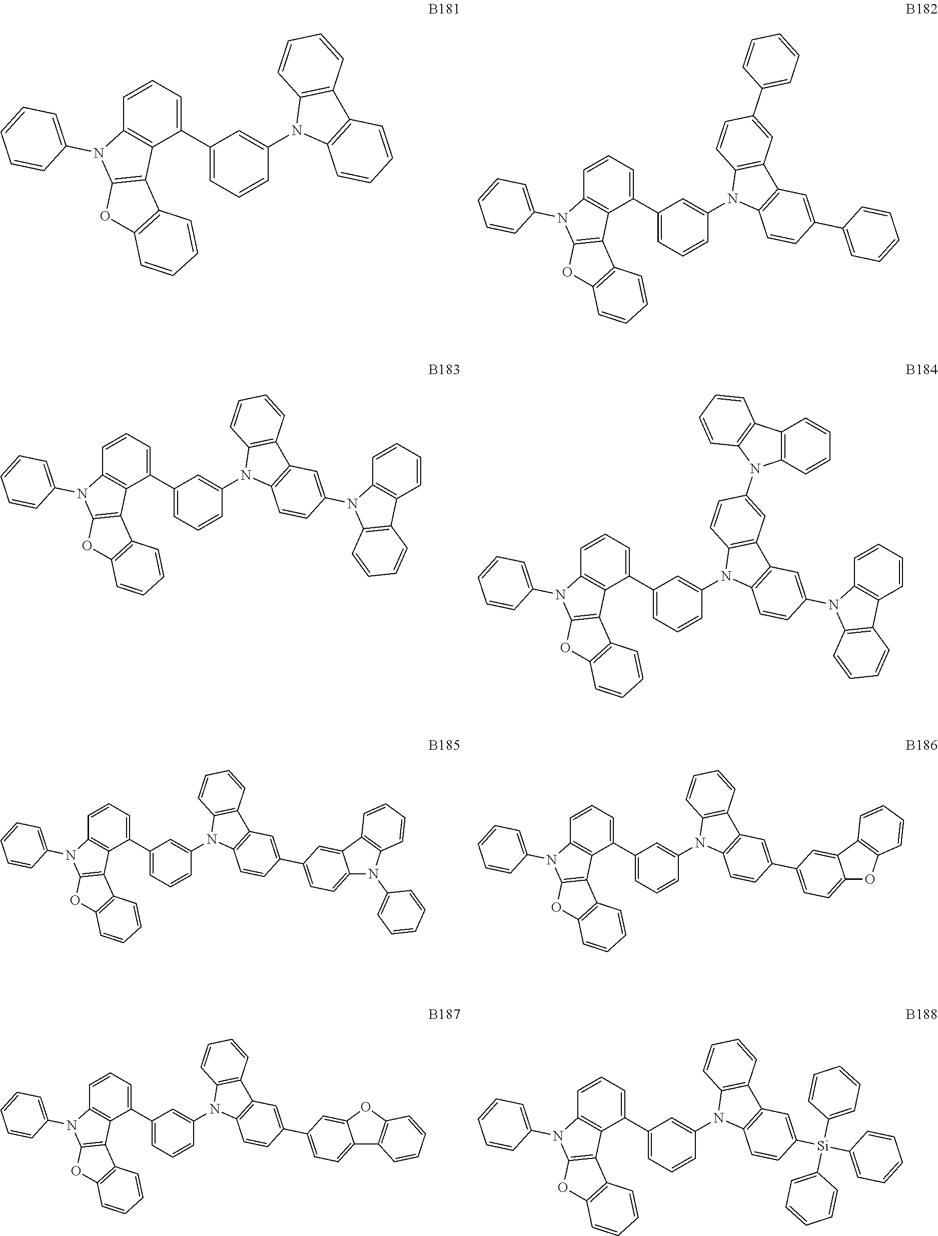

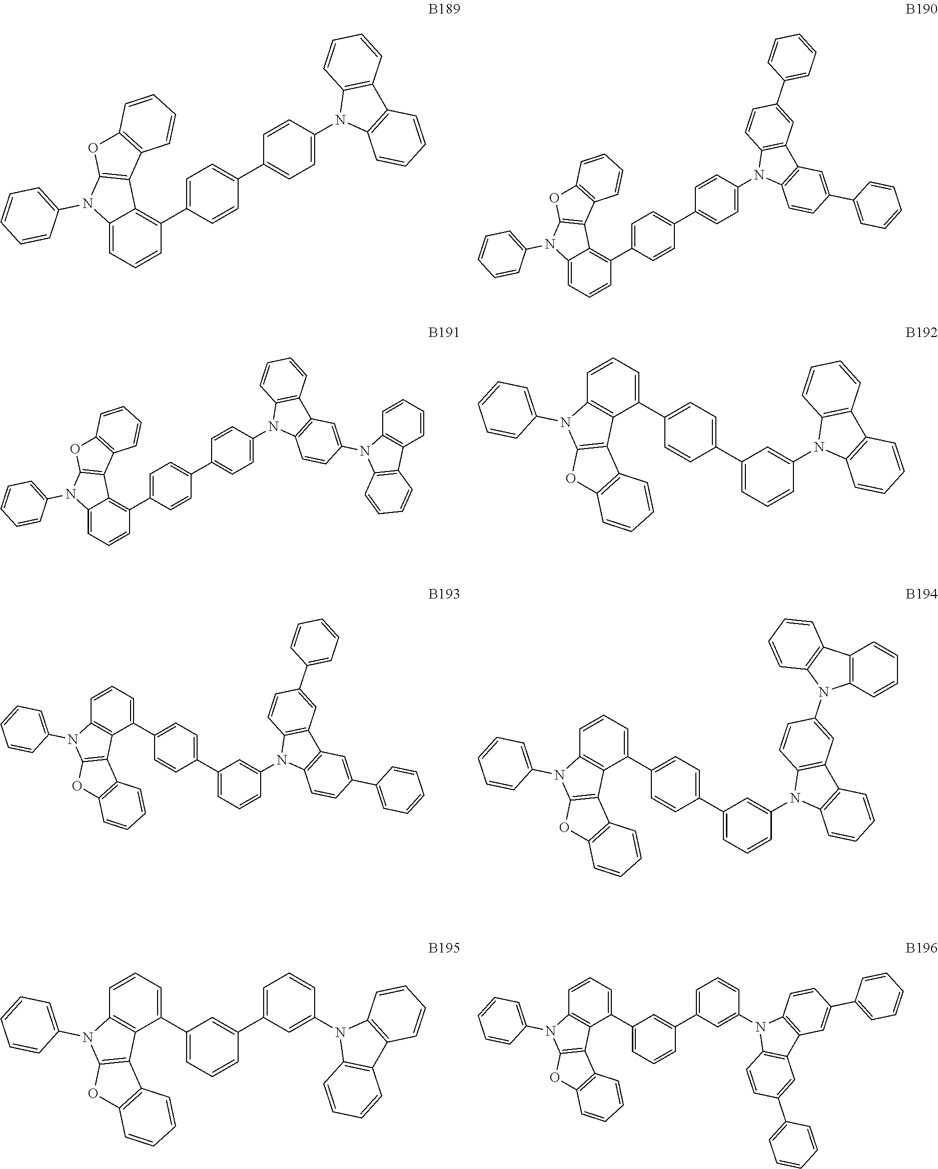

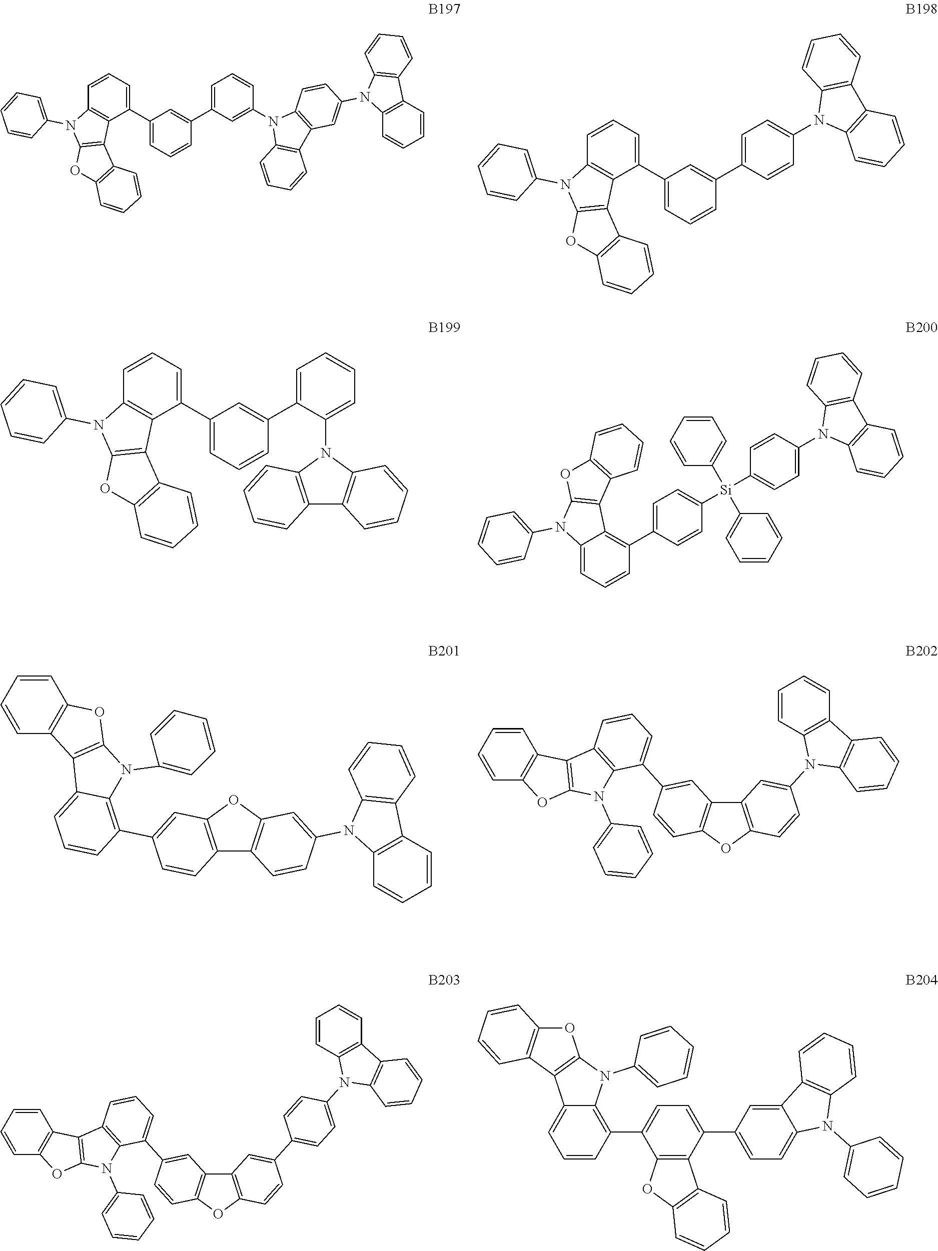

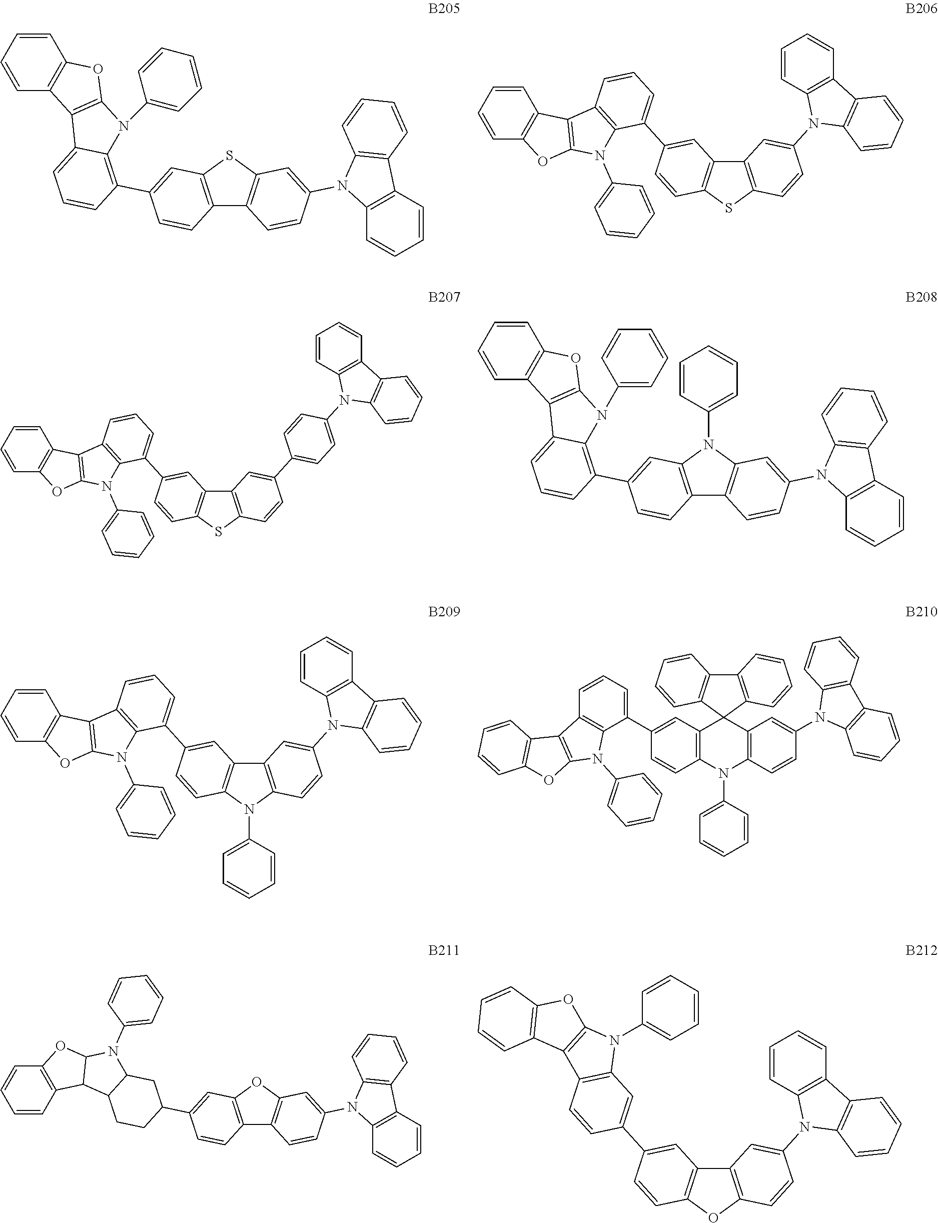

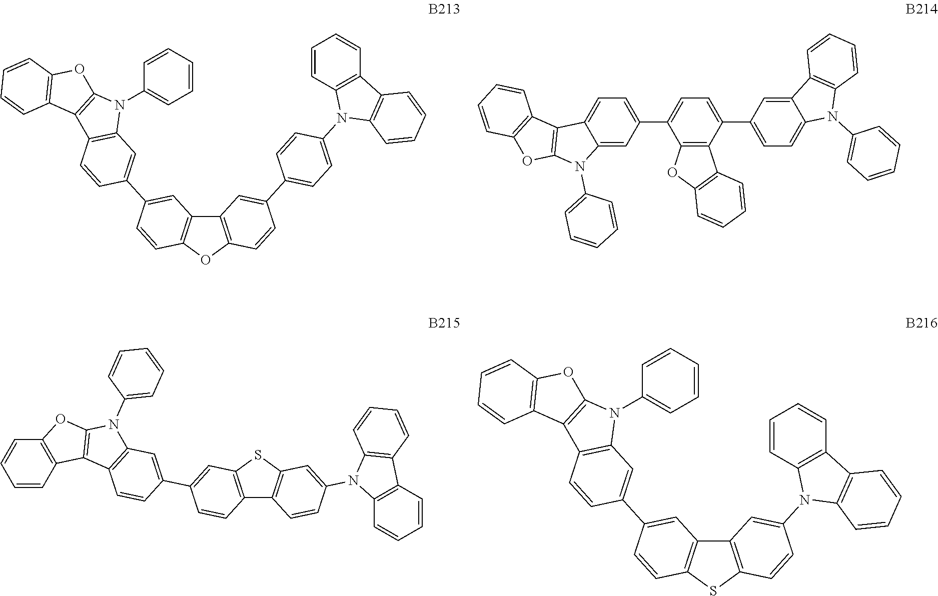 [SMILES: c1ccc(-n2c3cc(-c4ccc5oc6ccc(-c7ccc(-n8c9ccccc9c9ccccc98)cc7)cc6c5c4)ccc3c3c4ccccc4oc32)cc1 ; c1ccc(-n2c3ccccc3c3cc(-c4ccc(-c5ccc6c7c8ccccc8oc7n(-c7ccccc7)c6c5)c5oc6ccccc6c45)ccc32)cc1 ; c1ccc(-n2c3cc(-c4ccc5c(c4)sc4cc(-n6c7ccccc7c7ccccc76)ccc45)ccc3c3c4ccccc4oc32)cc1 ; c1ccc(-n2c3cc(-c4ccc5sc6ccc(-n7c8ccccc8c8ccccc87)cc6c5c4)ccc3c3c4ccccc4oc32)cc1]

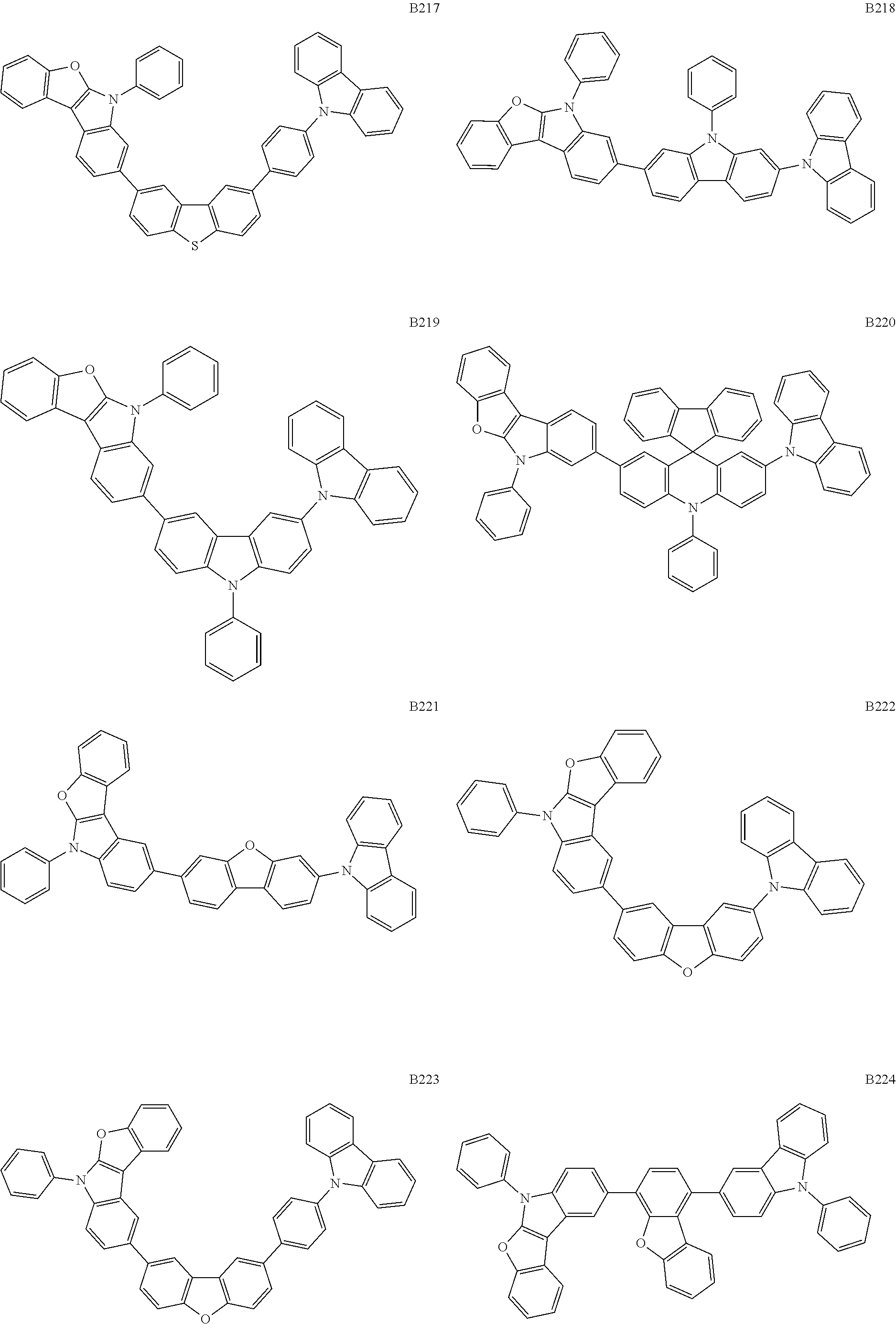

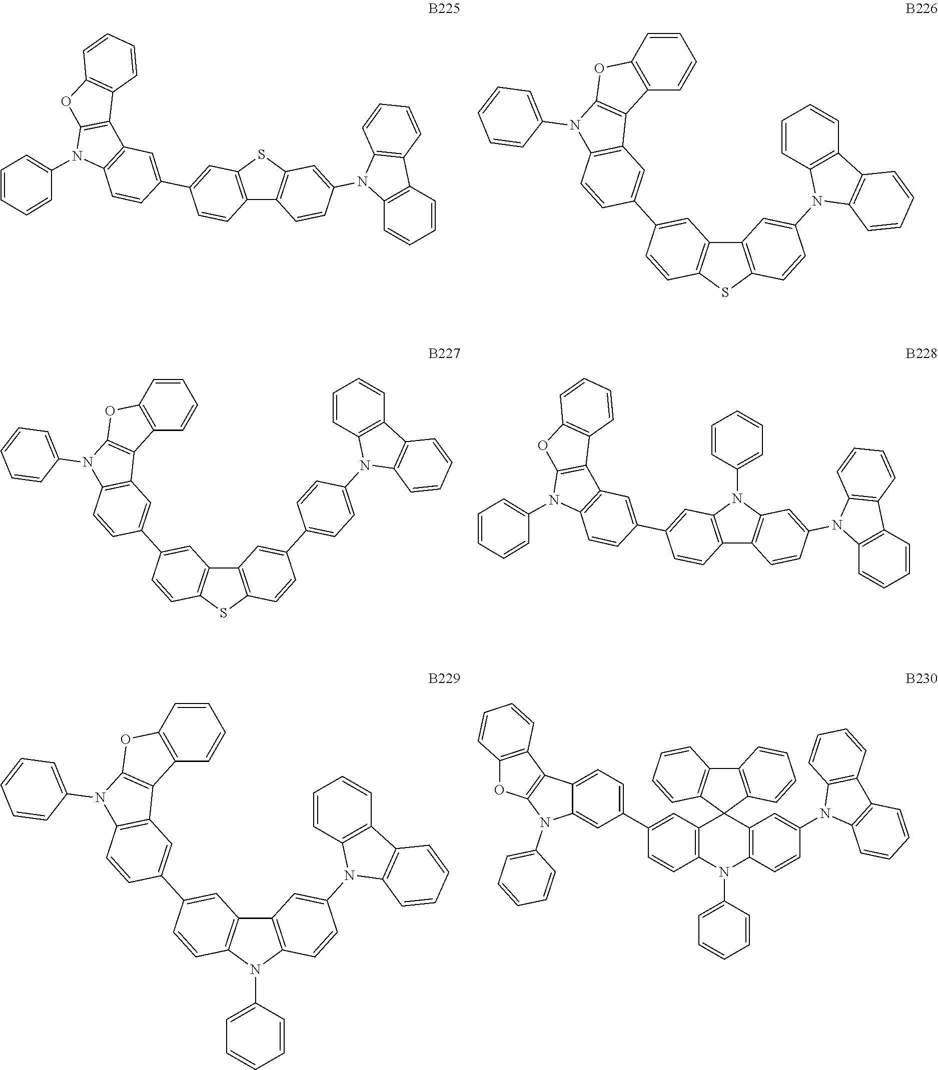

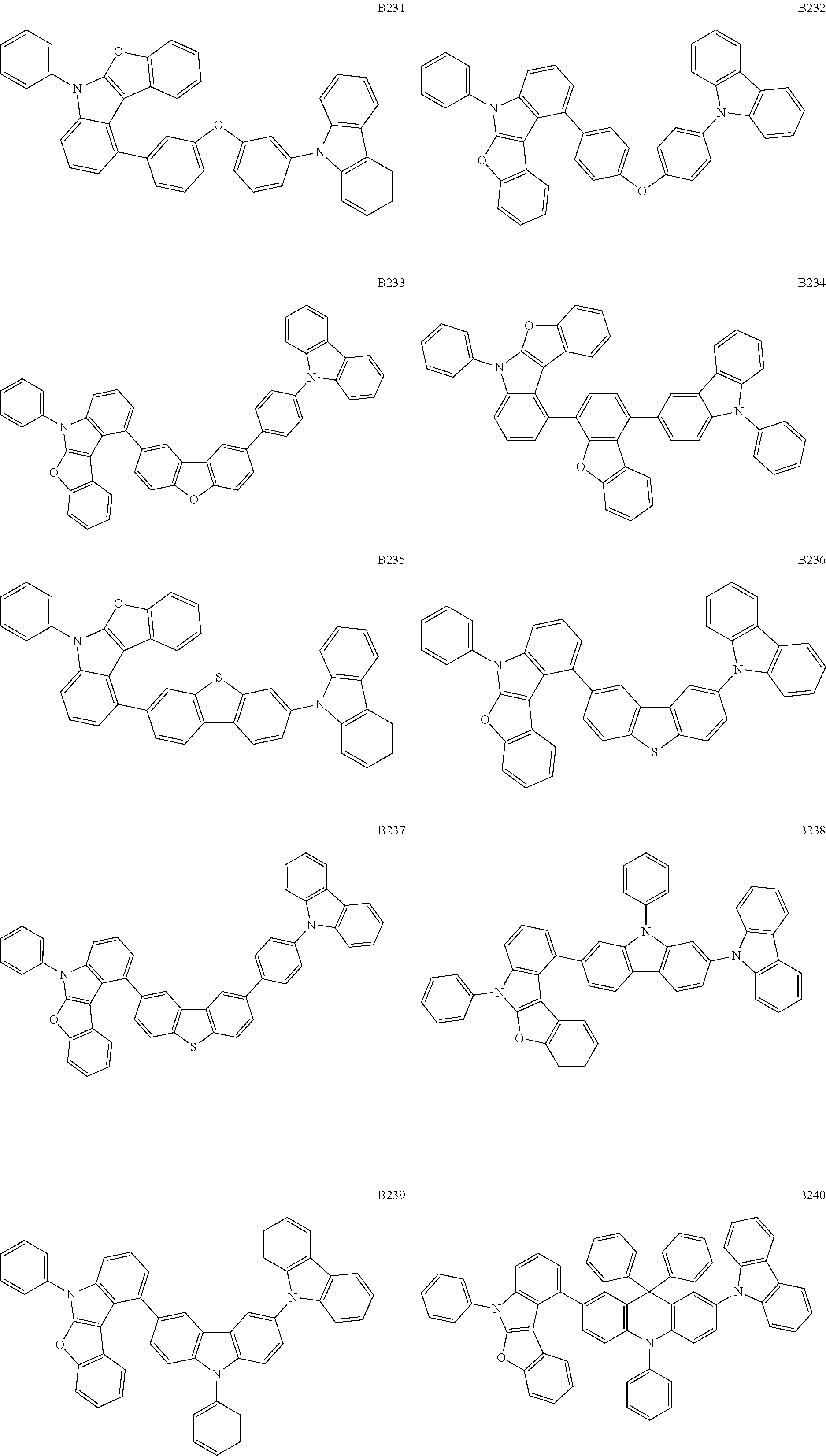

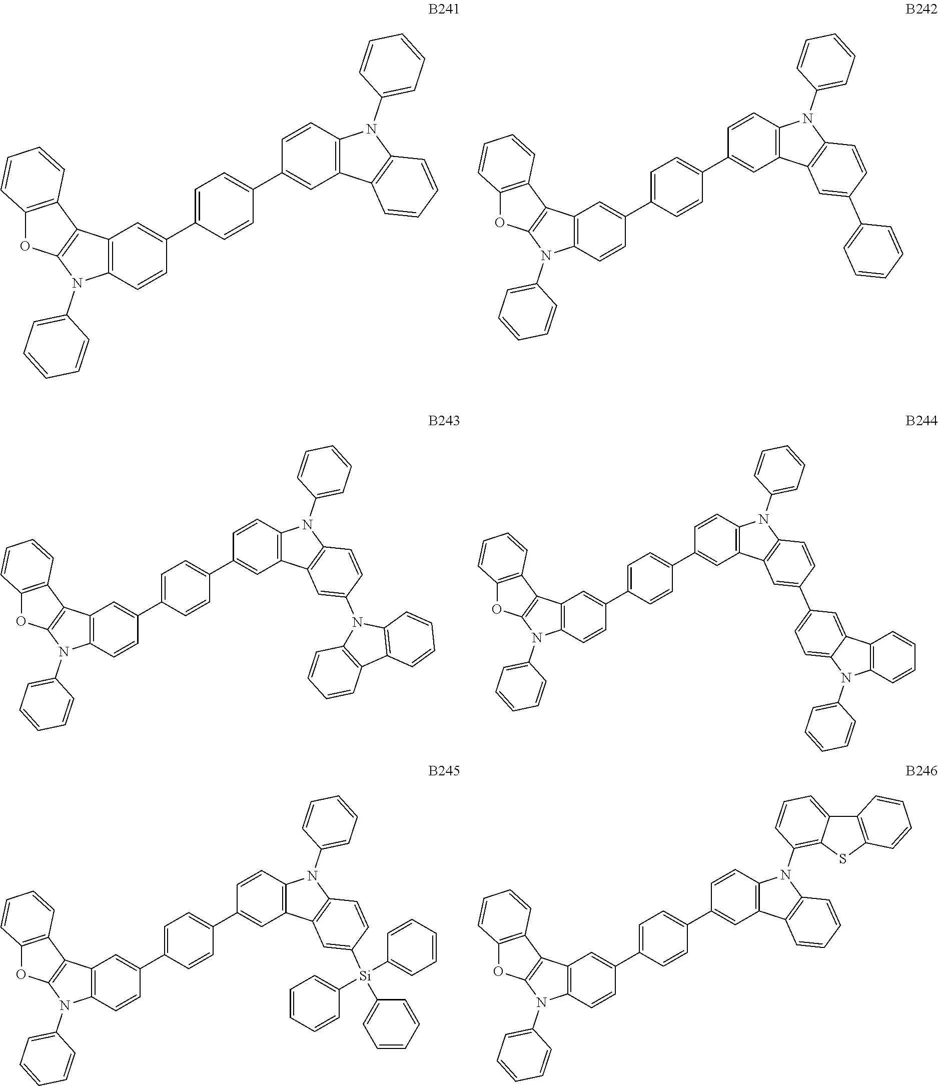

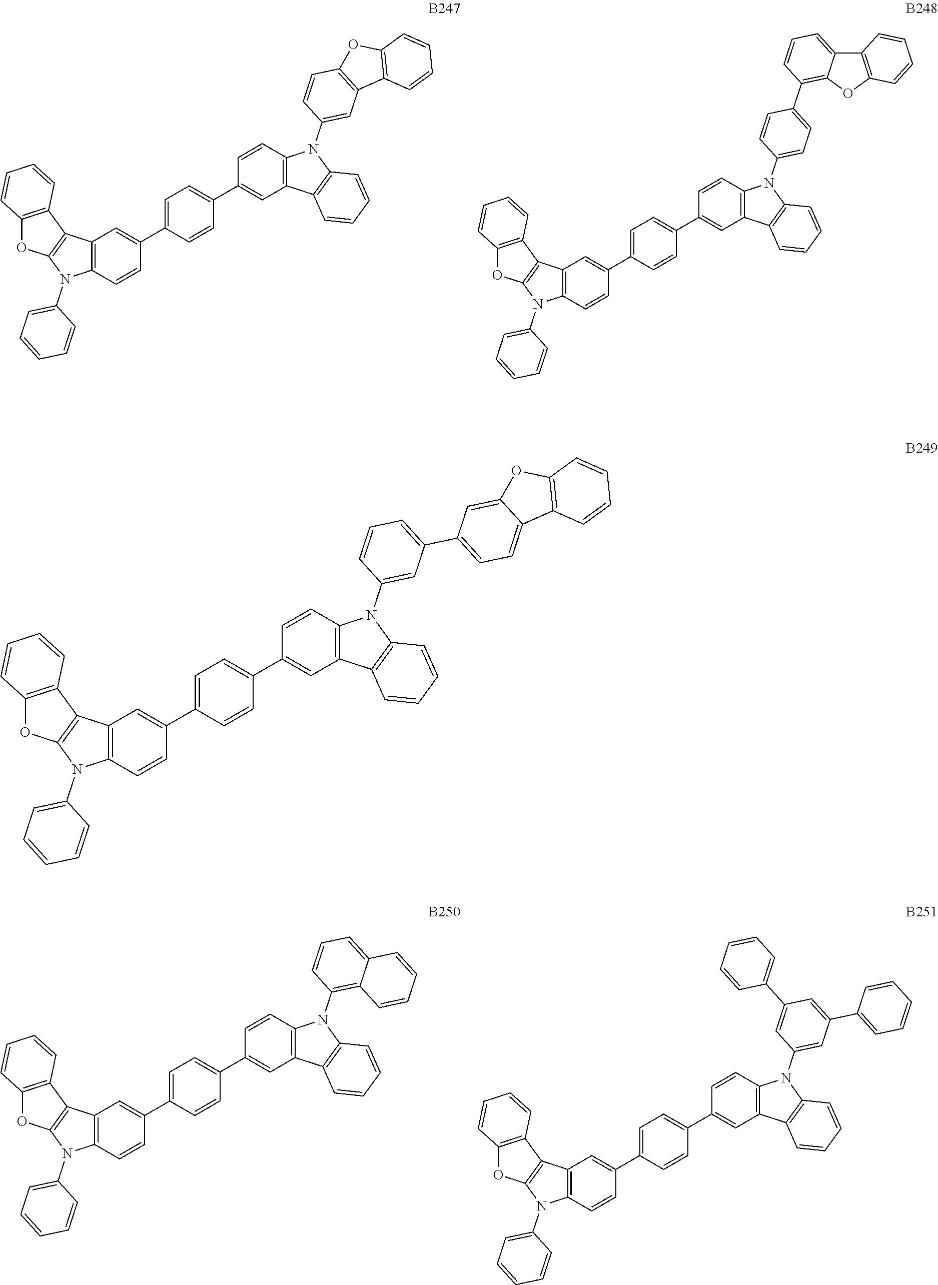 [SMILES: c1ccc(-n2c3ccc(-c4ccc(-c5ccc6c(c5)c5ccccc5n6-c5ccc6oc7ccccc7c6c5)cc4)cc3c3c4ccccc4oc32)cc1 ; c1ccc(-n2c3ccc(-c4ccc(-c5ccc6c(c5)c5ccccc5n6-c5ccc(-c6cccc7c6oc6ccccc67)cc5)cc4)cc3c3c4ccccc4oc32)cc1 ; c1ccc(-n2c3ccc(-c4ccc(-c5ccc6c(c5)c5ccccc5n6-c5cccc(-c6ccc7c(c6)oc6ccccc67)c5)cc4)cc3c3c4ccccc4oc32)cc1 ; c1ccc(-n2c3ccc(-c4ccc(-c5ccc6c(c5)c5ccccc5n6-c5cccc6ccccc56)cc4)cc3c3c4ccccc4oc32)cc1 ; c1ccc(-c2cc(-c3ccccc3)cc(-n3c4ccccc4c4cc(-c5ccc(-c6ccc7c(c6)c6c8ccccc8oc6n7-c6ccccc6)cc5)ccc43)c2)cc1]

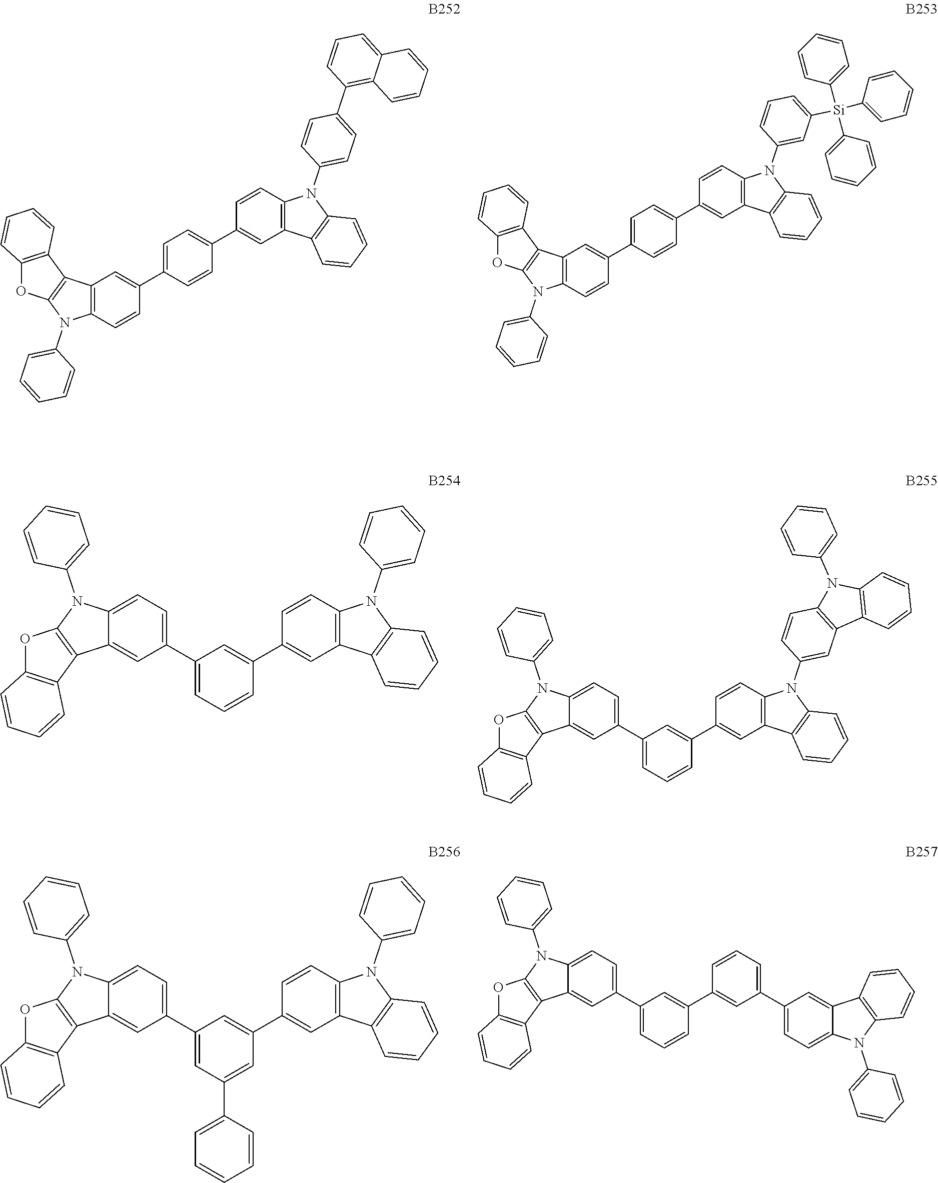

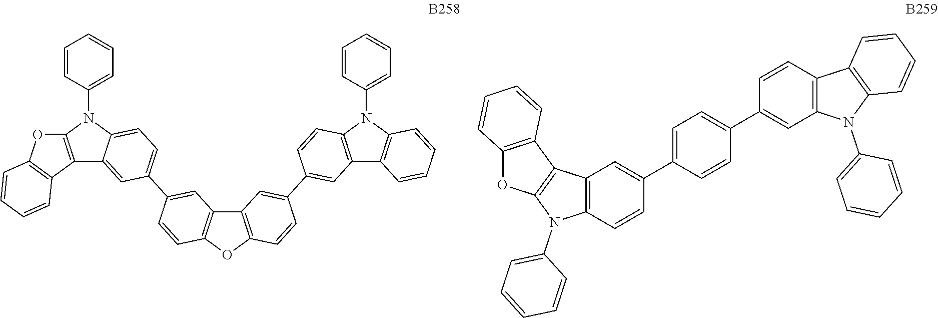 [SMILES: c1ccc(-n2c3ccccc3c3cc(-c4ccc5oc6ccc(-c7ccc8c(c7)c7c9ccccc9oc7n8-c7ccccc7)cc6c5c4)ccc32)cc1 ; c1ccc(-n2c3ccccc3c3ccc(-c4ccc(-c5ccc6c(c5)c5c7ccccc7oc5n6-c5ccccc5)cc4)cc32)cc1]

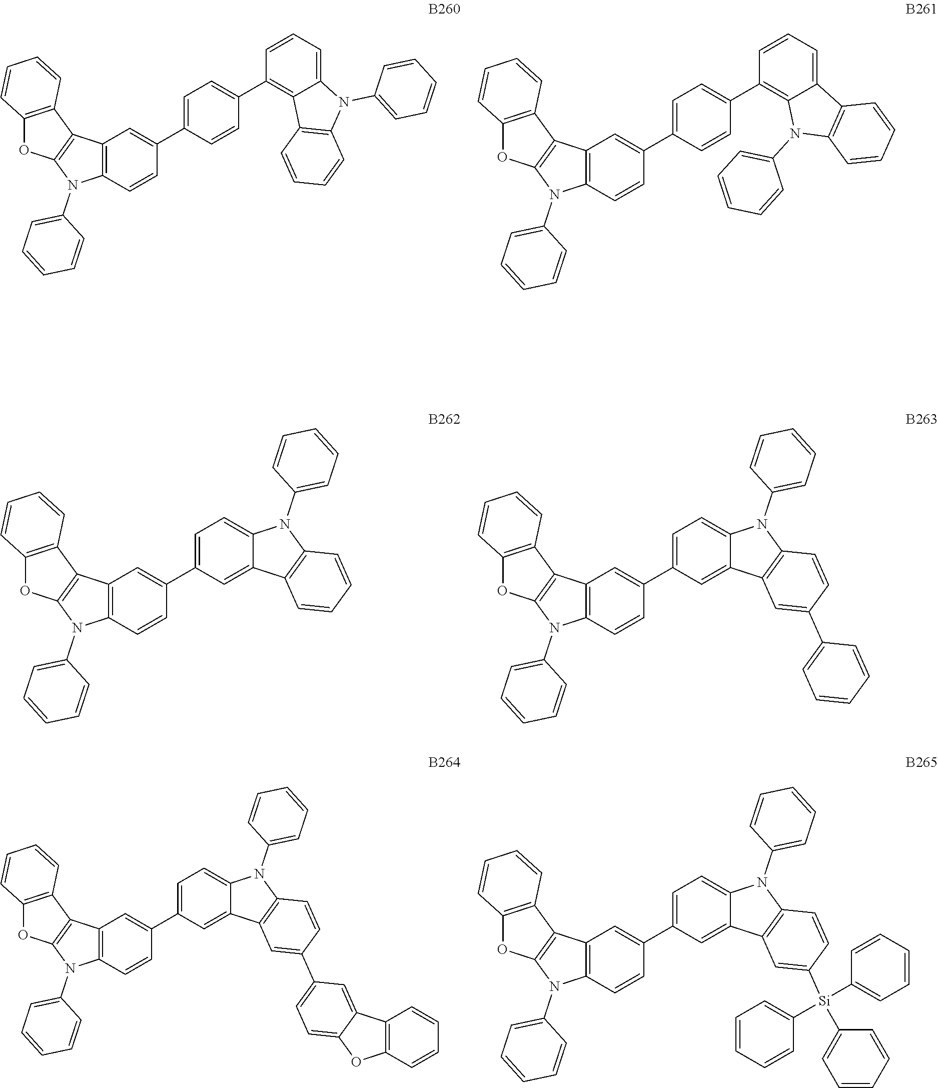

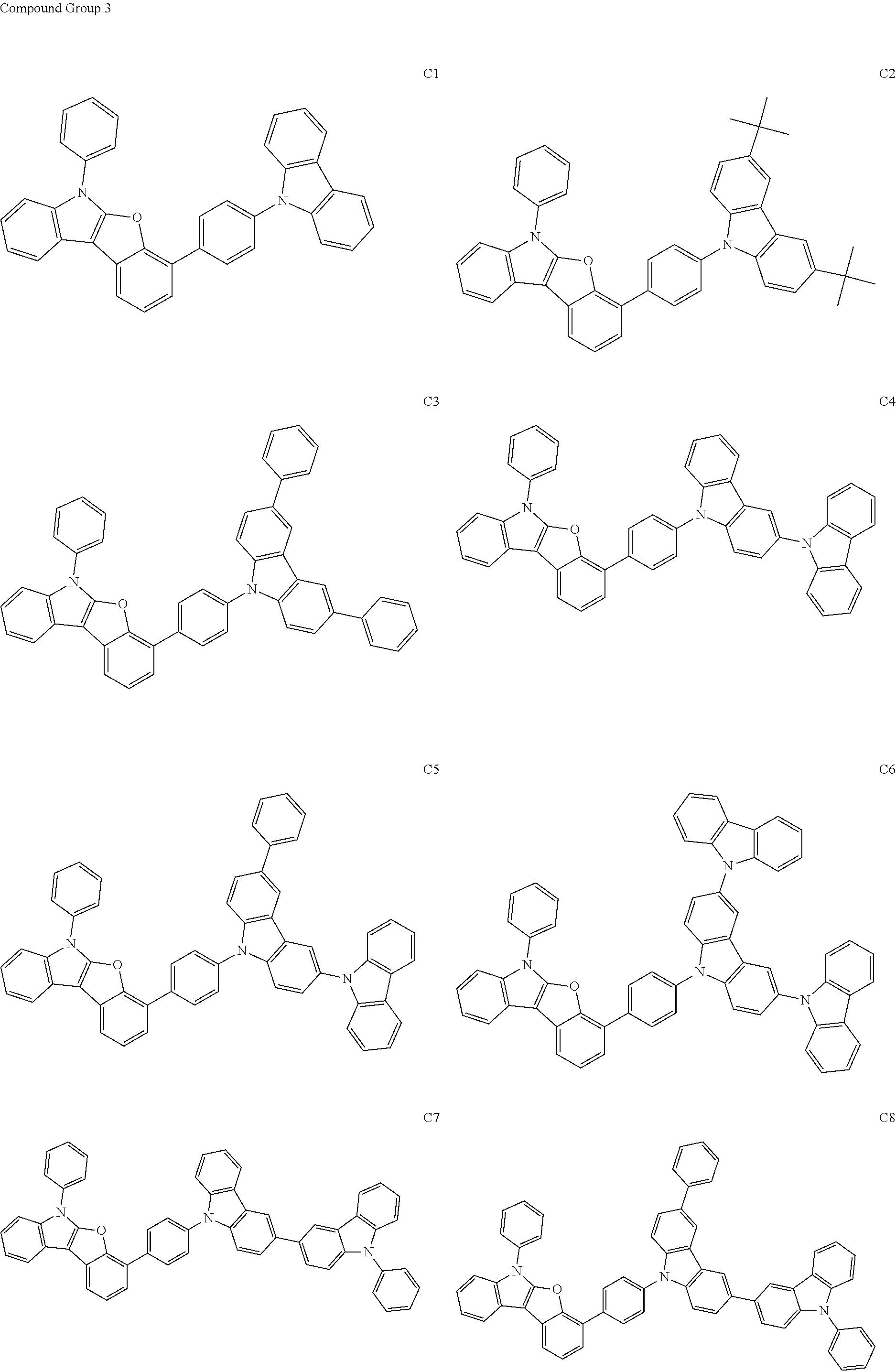

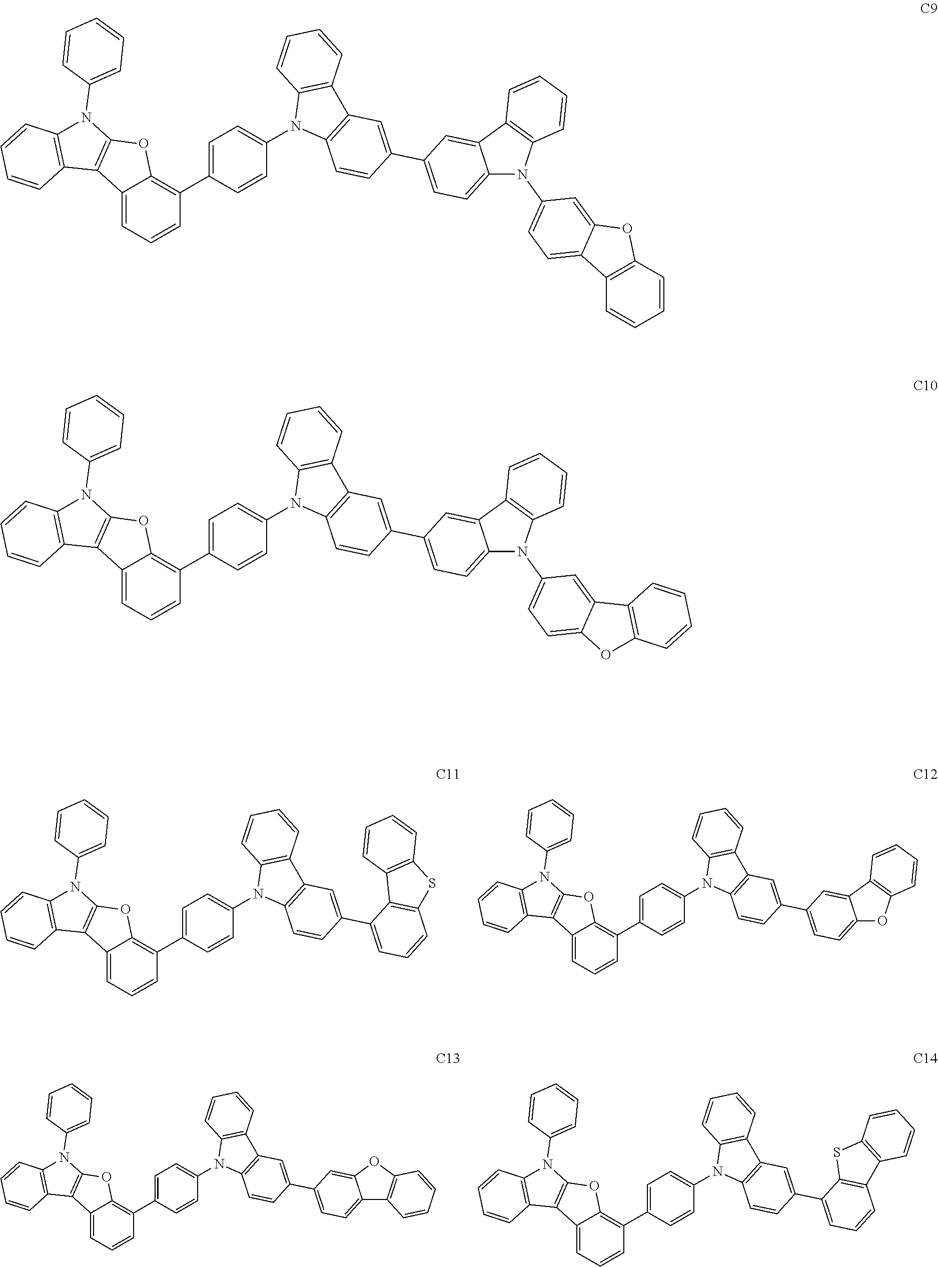

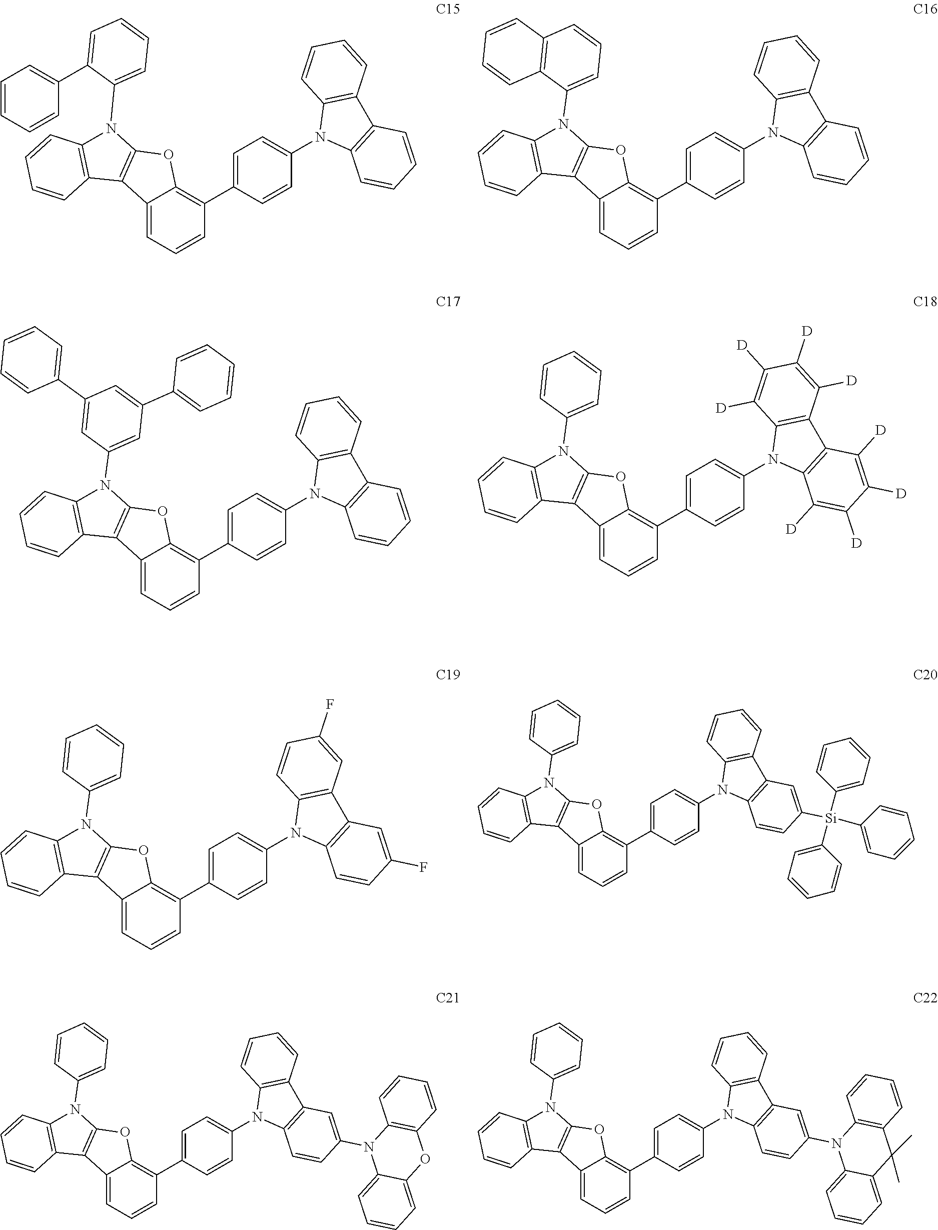

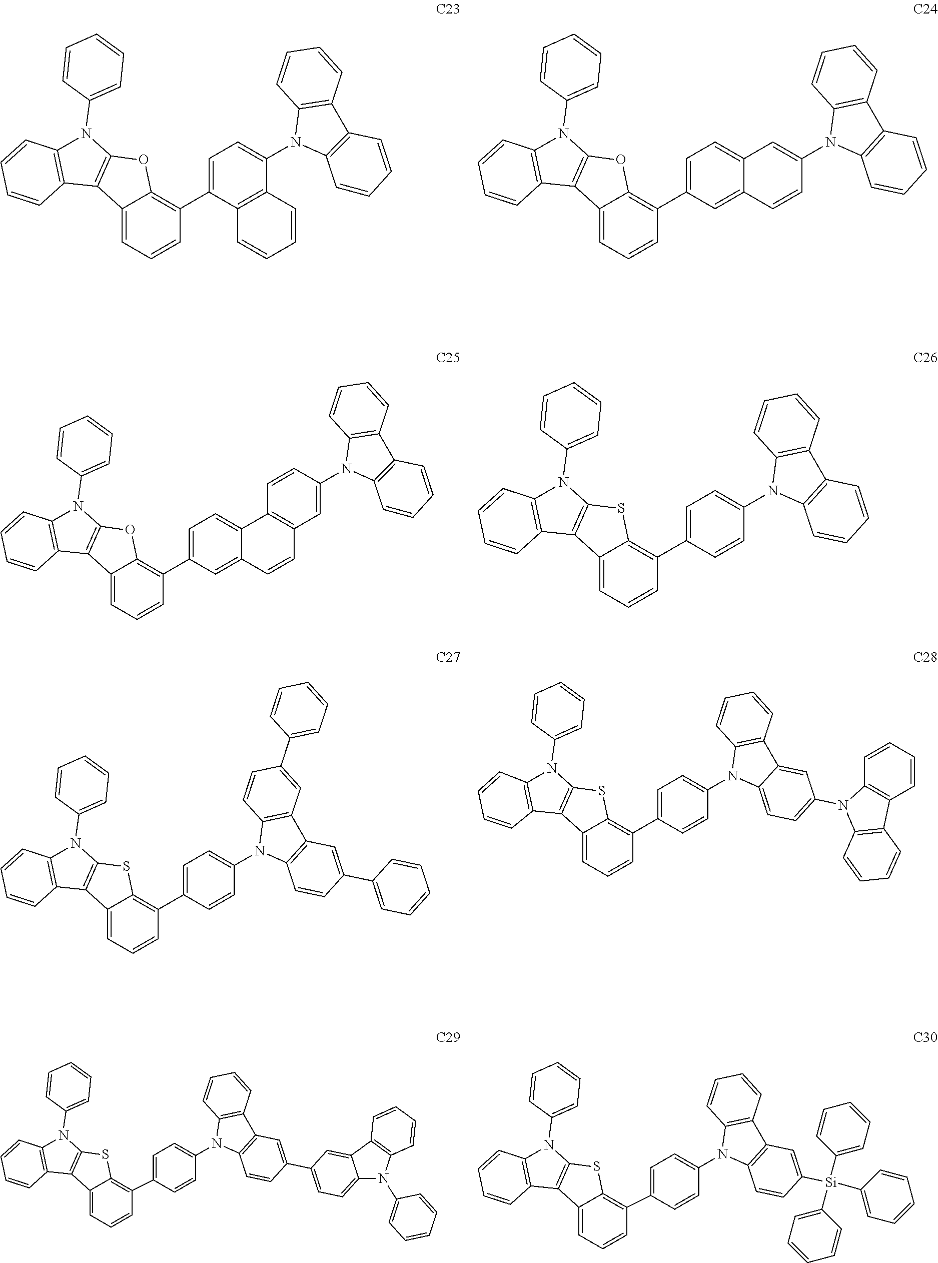

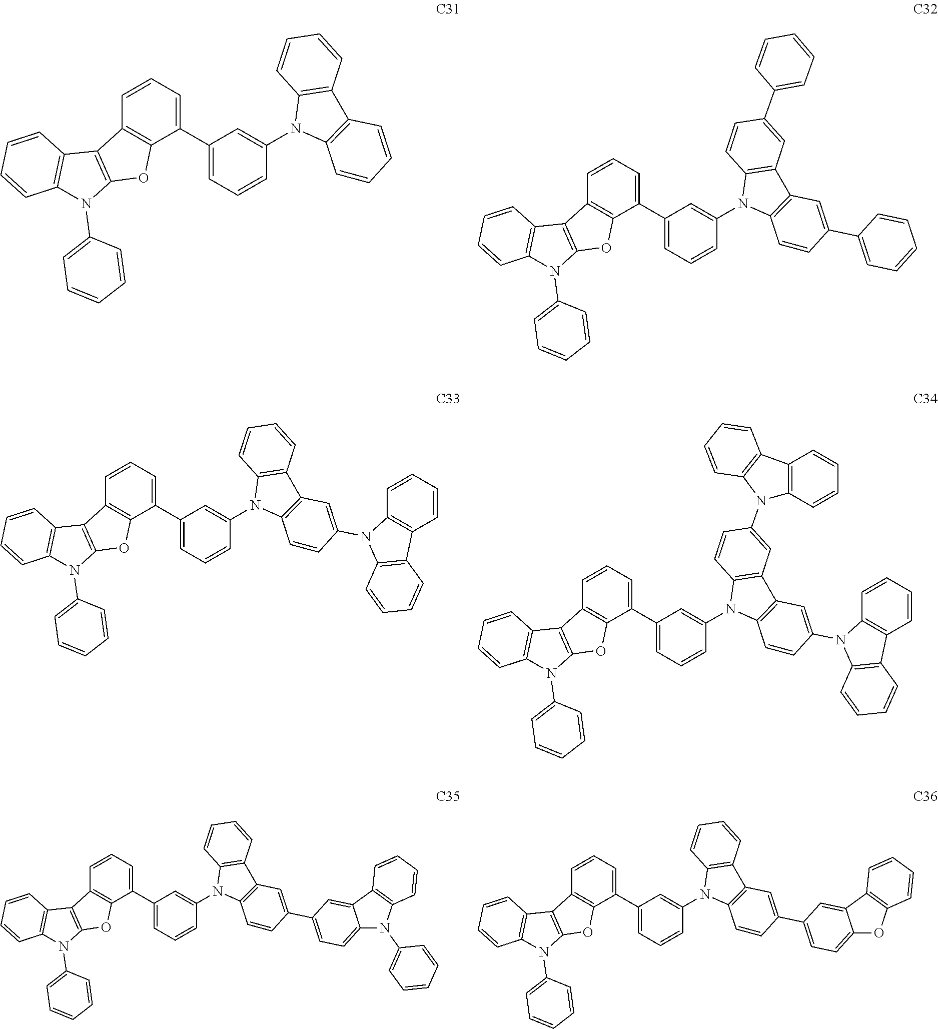

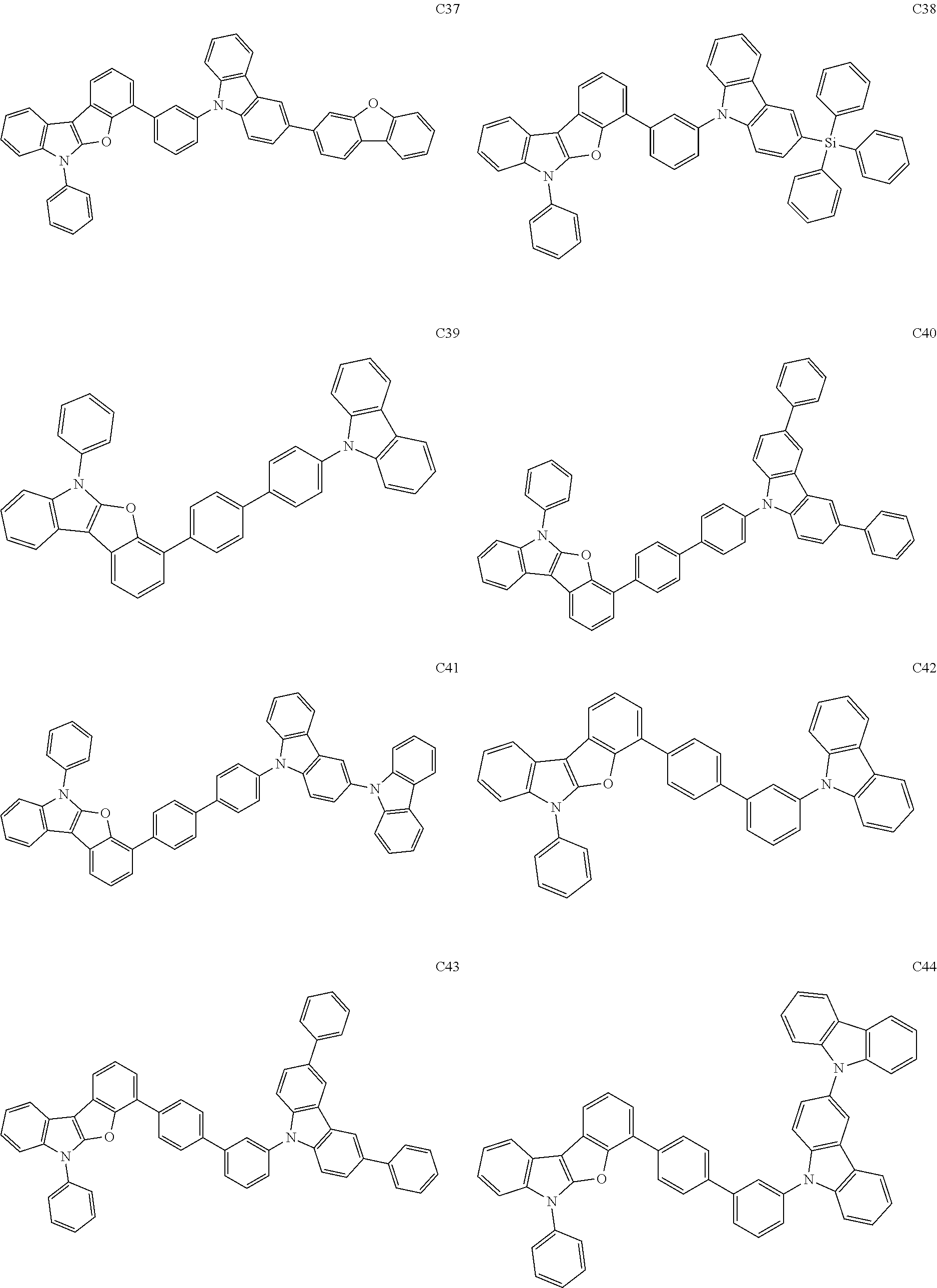

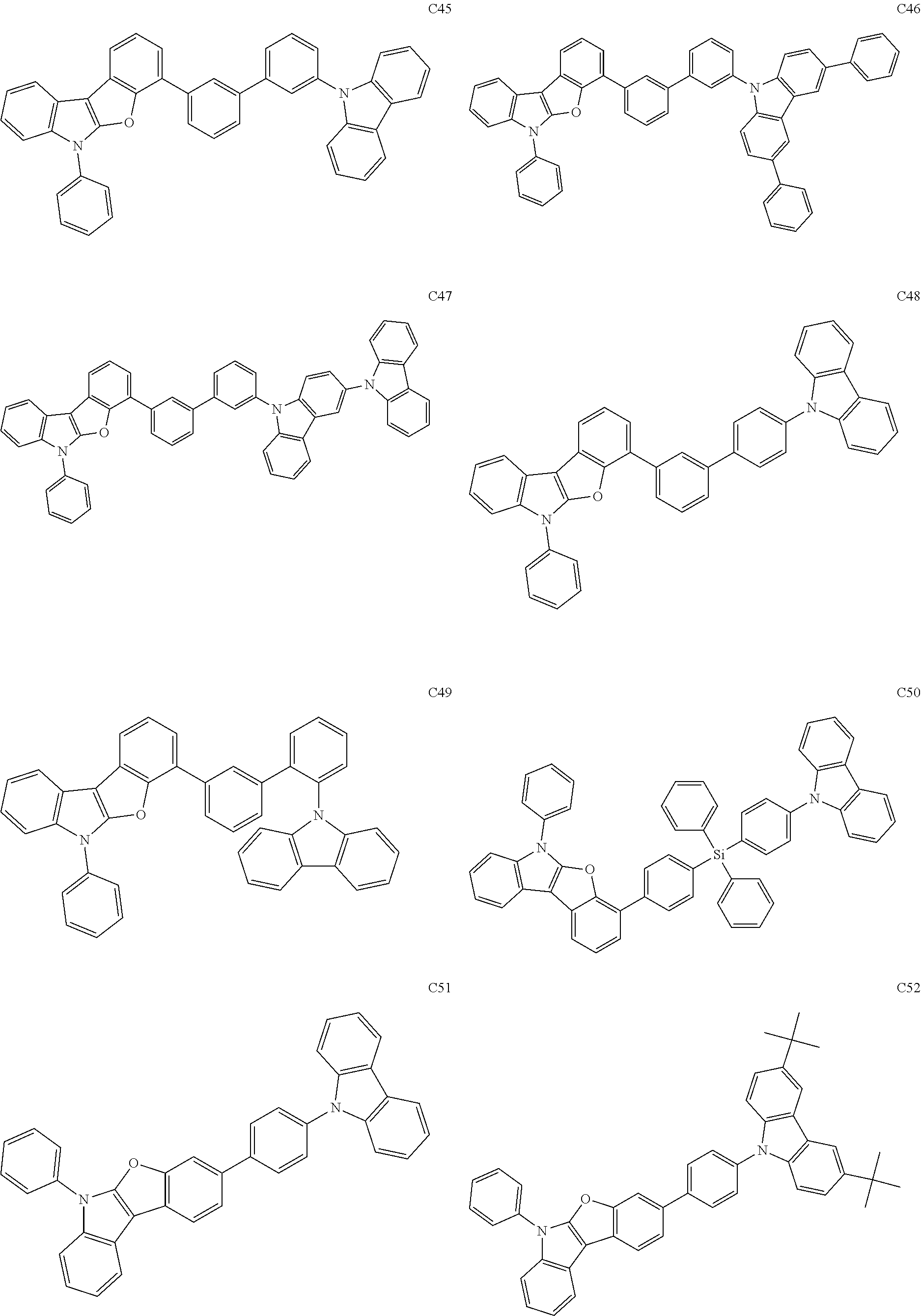

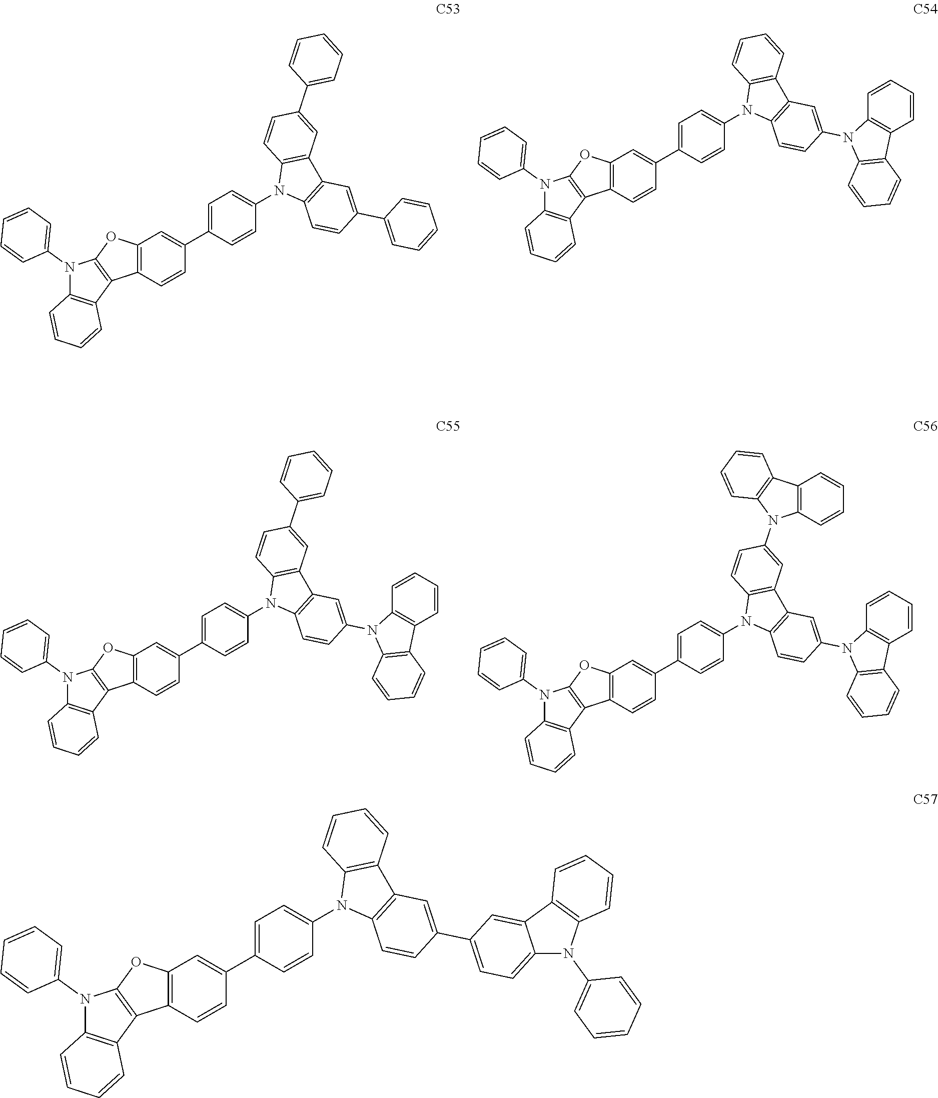

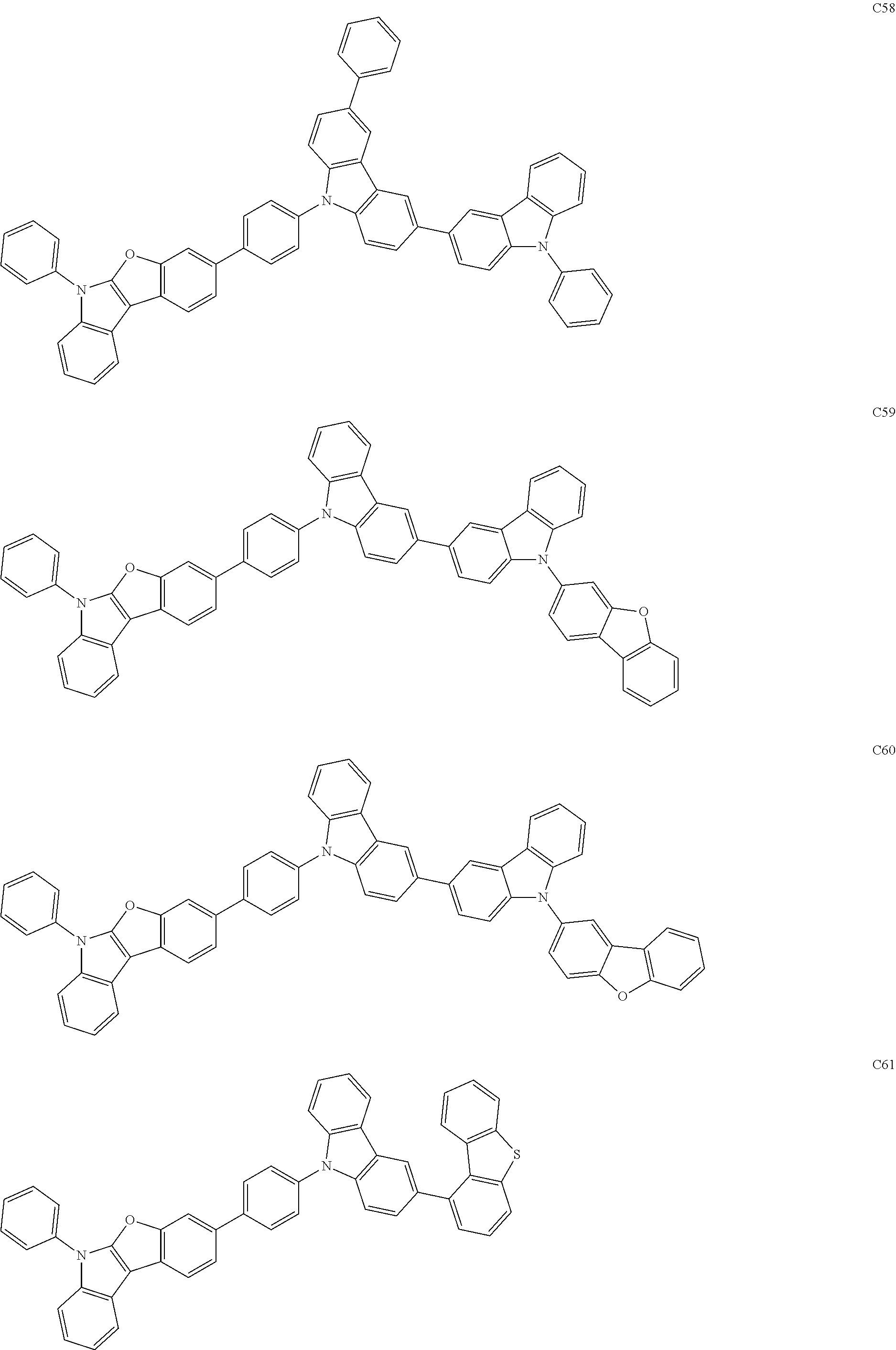 [SMILES: c1ccc(-c2ccc3c(c2)c2cc(-c4ccc5c(c4)c4ccccc4n5-c4ccccc4)ccc2n3-c2ccc(-c3ccc4c(c3)oc3c4c4ccccc4n3-c3ccccc3)cc2)cc1 ; c1ccc(-n2c3ccccc3c3c4ccc(-c5ccc(-n6c7ccccc7c7cc(-c8ccc9c(c8)c8ccccc8n9-c8ccc9c(c8)oc8ccccc89)ccc76)cc5)cc4oc32)cc1 ; c1ccc(-n2c3ccccc3c3c4ccc(-c5ccc(-n6c7ccccc7c7cc(-c8ccc9c(c8)c8ccccc8n9-c8ccc9oc%10ccccc%10c9c8)ccc76)cc5)cc4oc32)cc1 ; c1ccc(-n2c3ccccc3c3c4ccc(-c5ccc(-n6c7ccccc7c7cc(-c8cccc9sc%10ccccc%10c89)ccc76)cc5)cc4oc32)cc1]

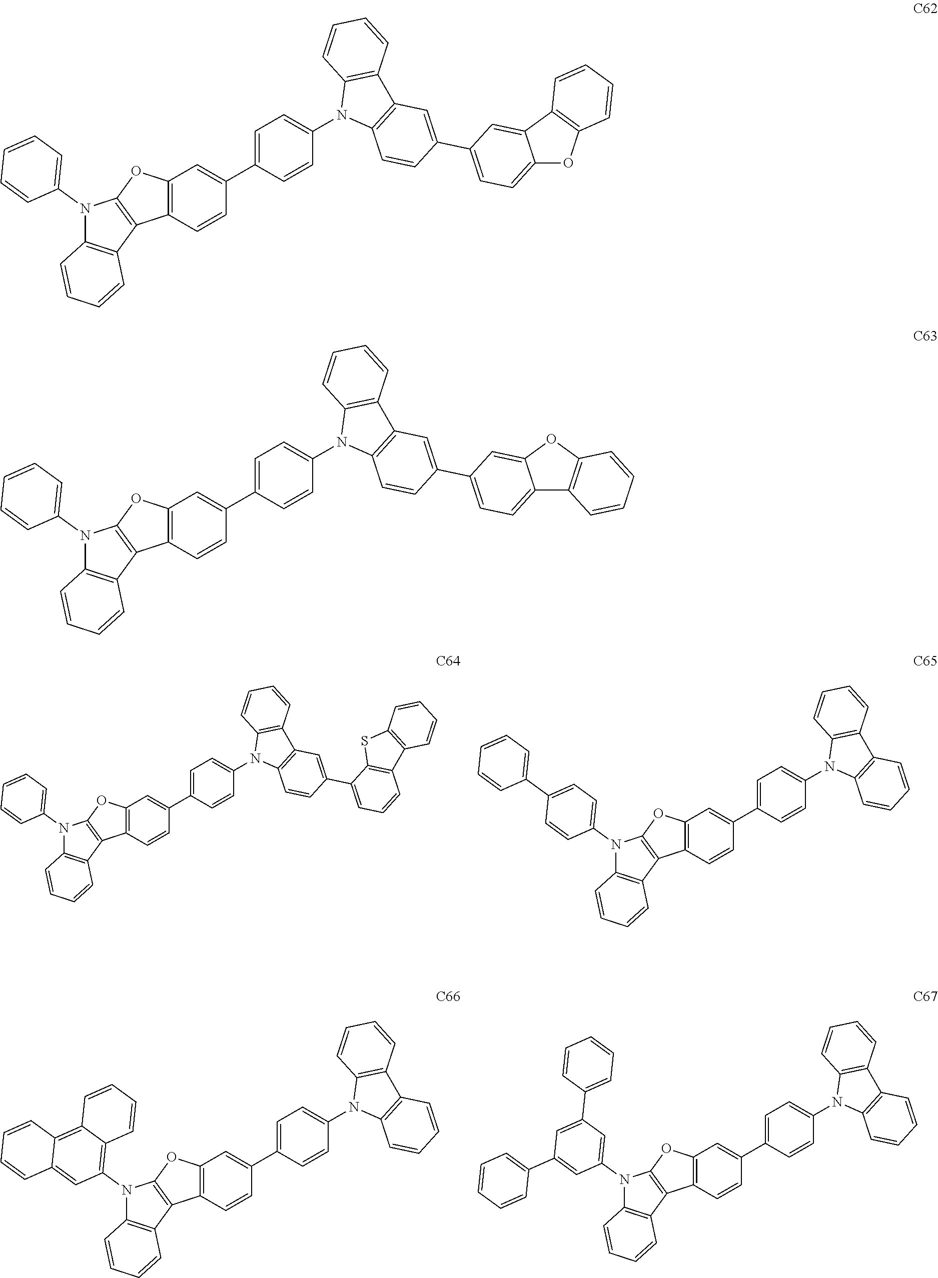

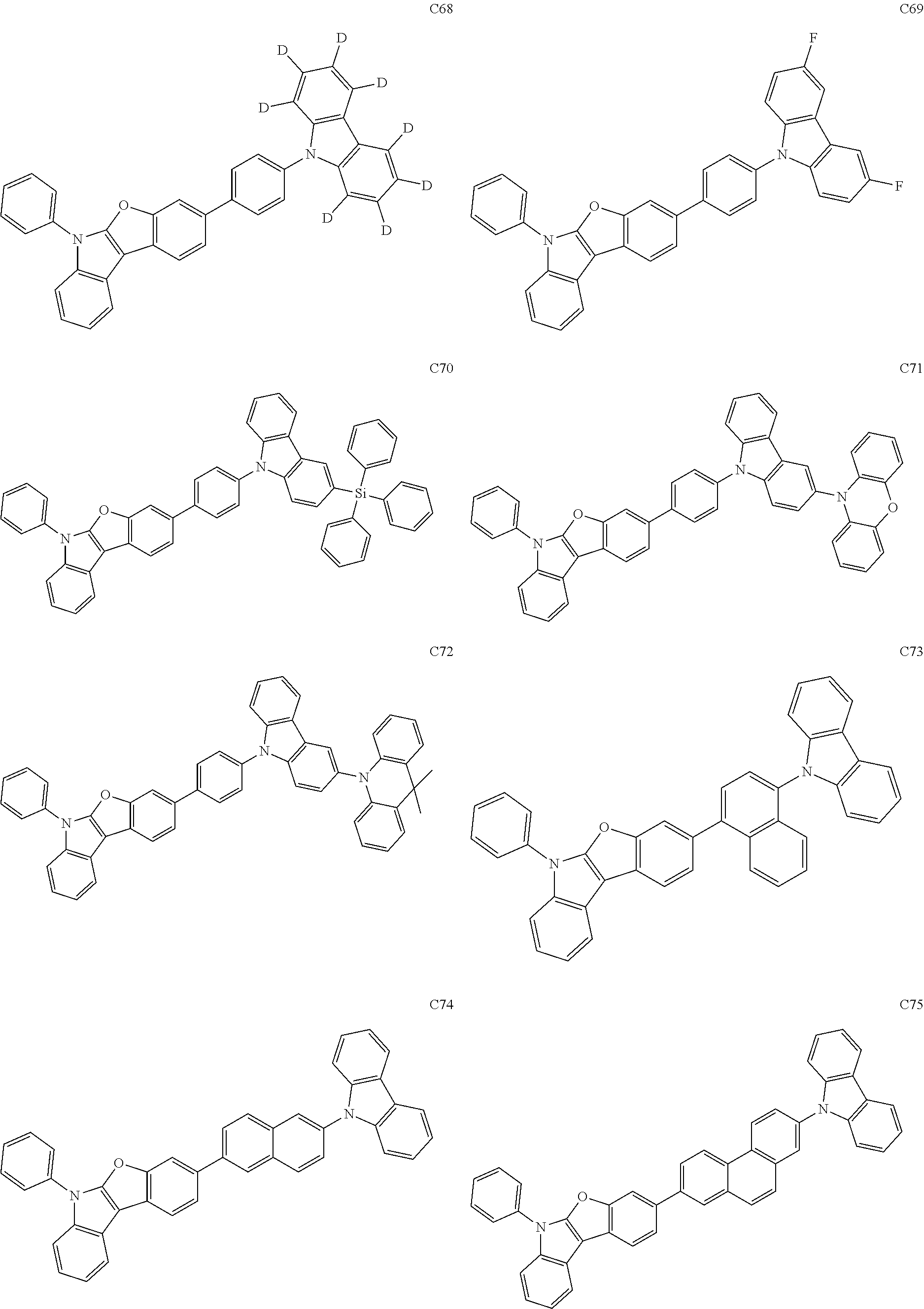

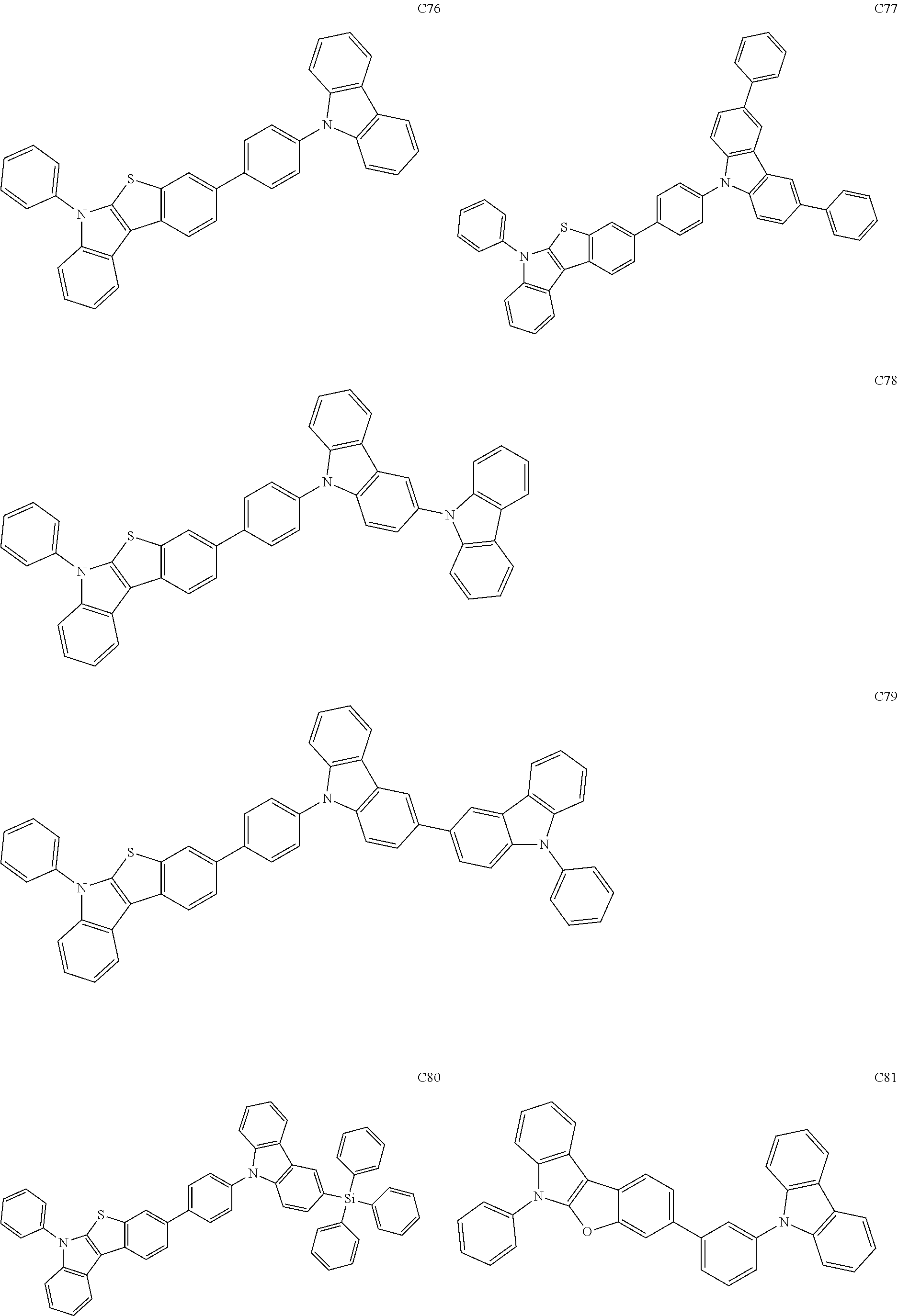

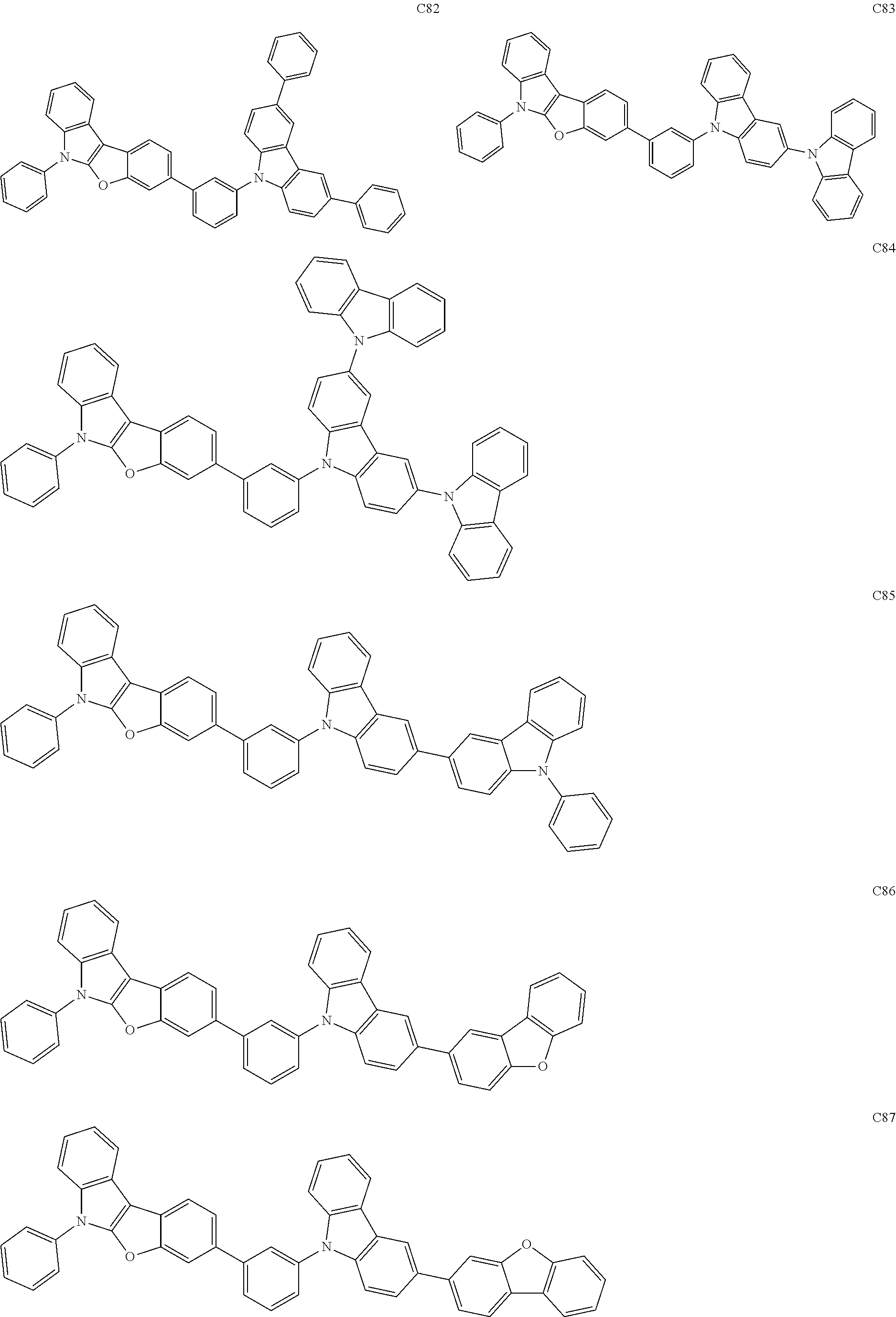

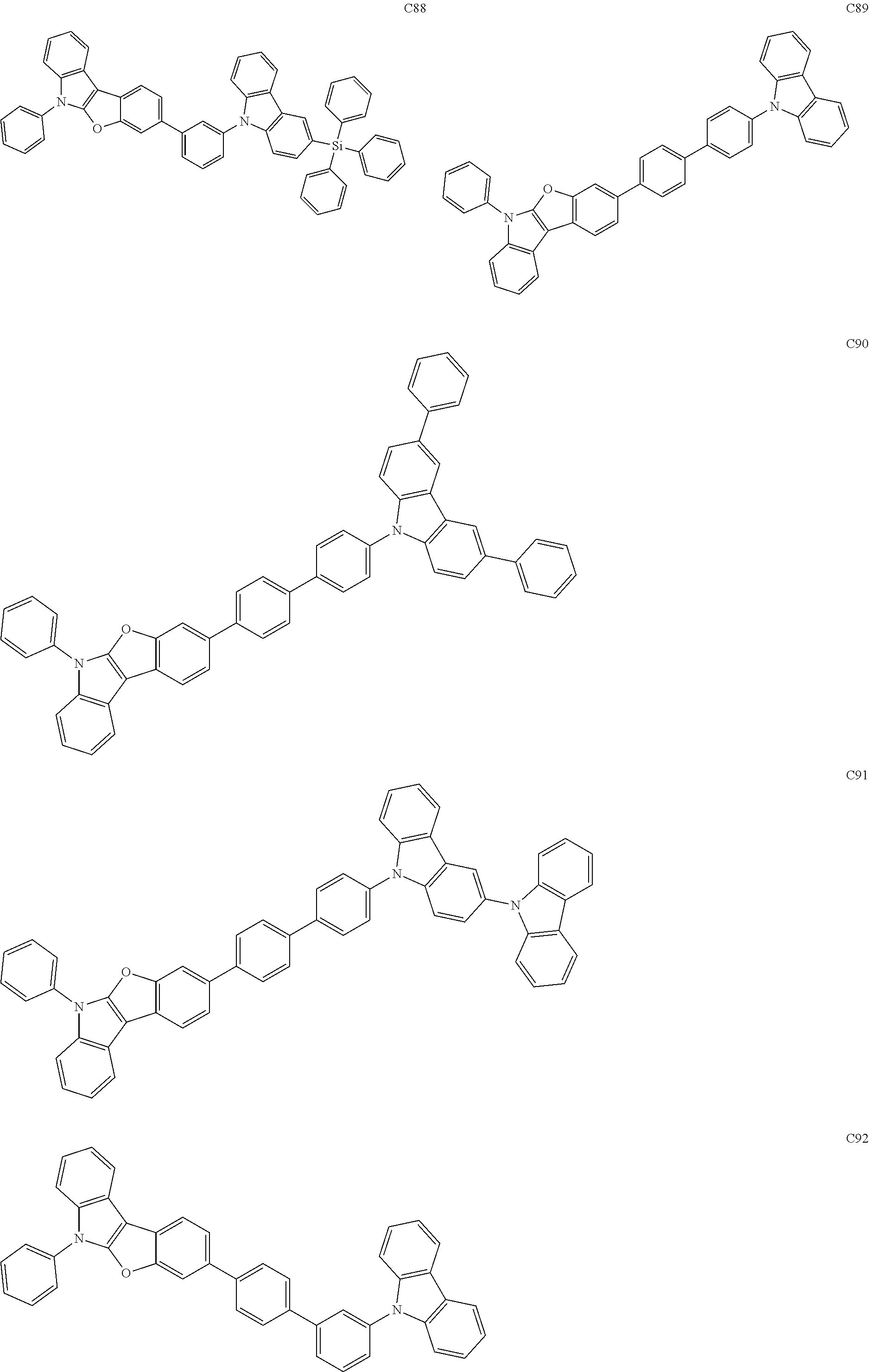

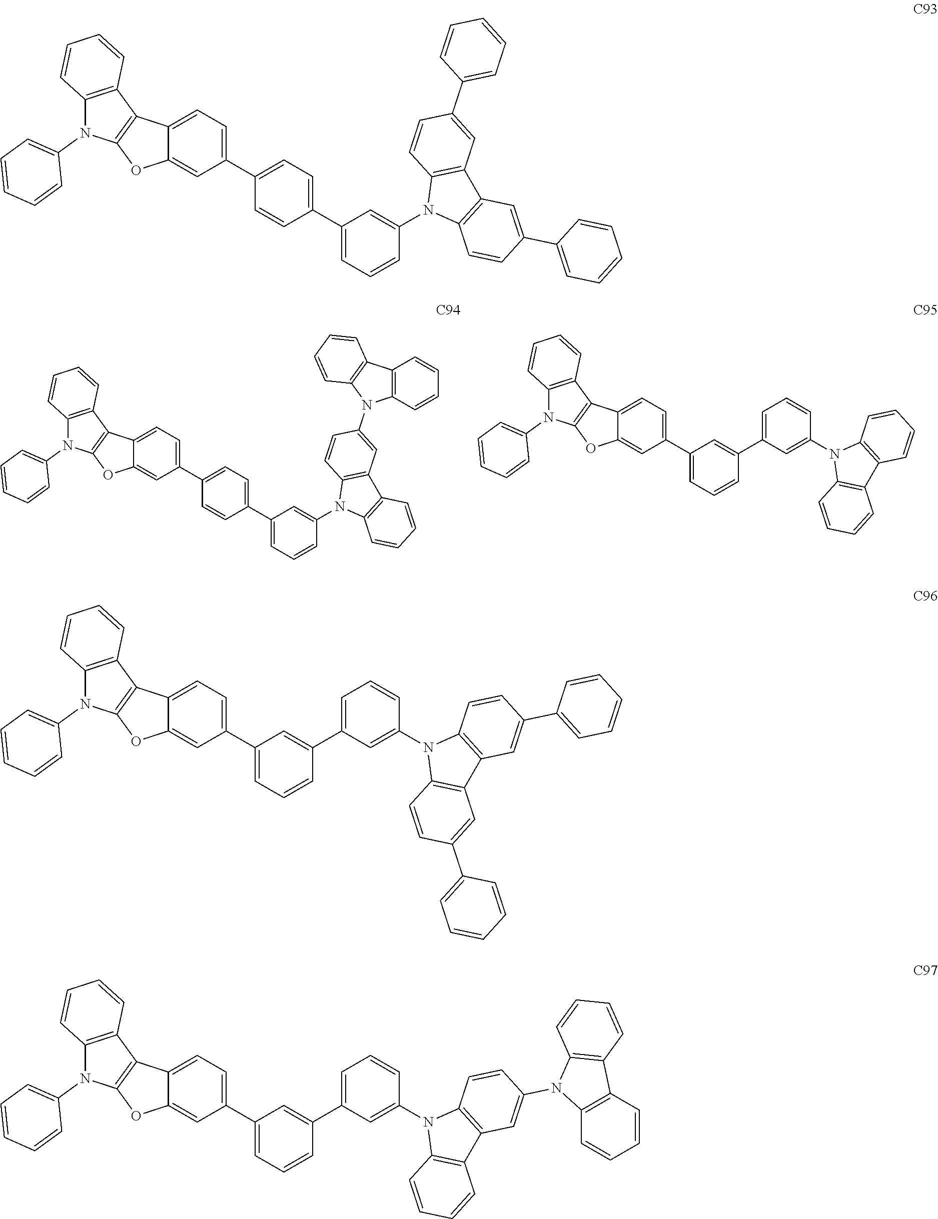

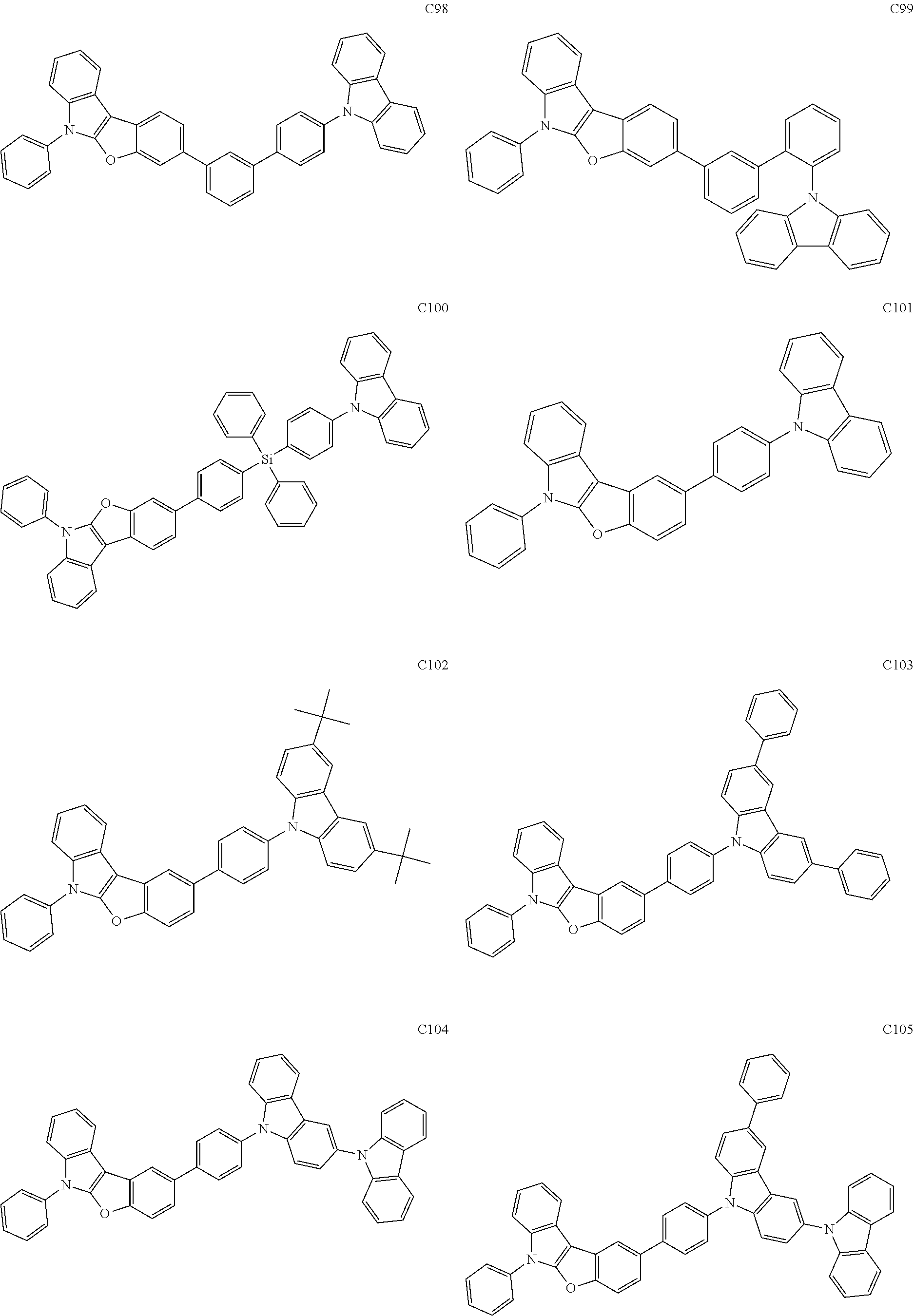

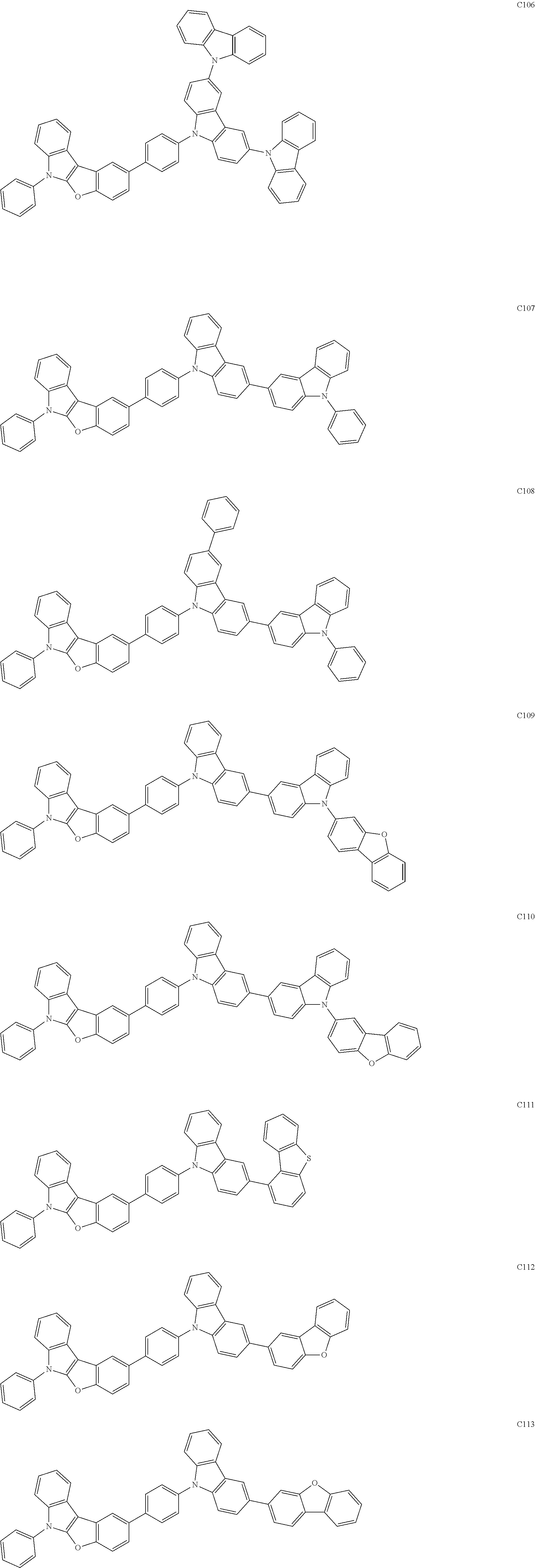

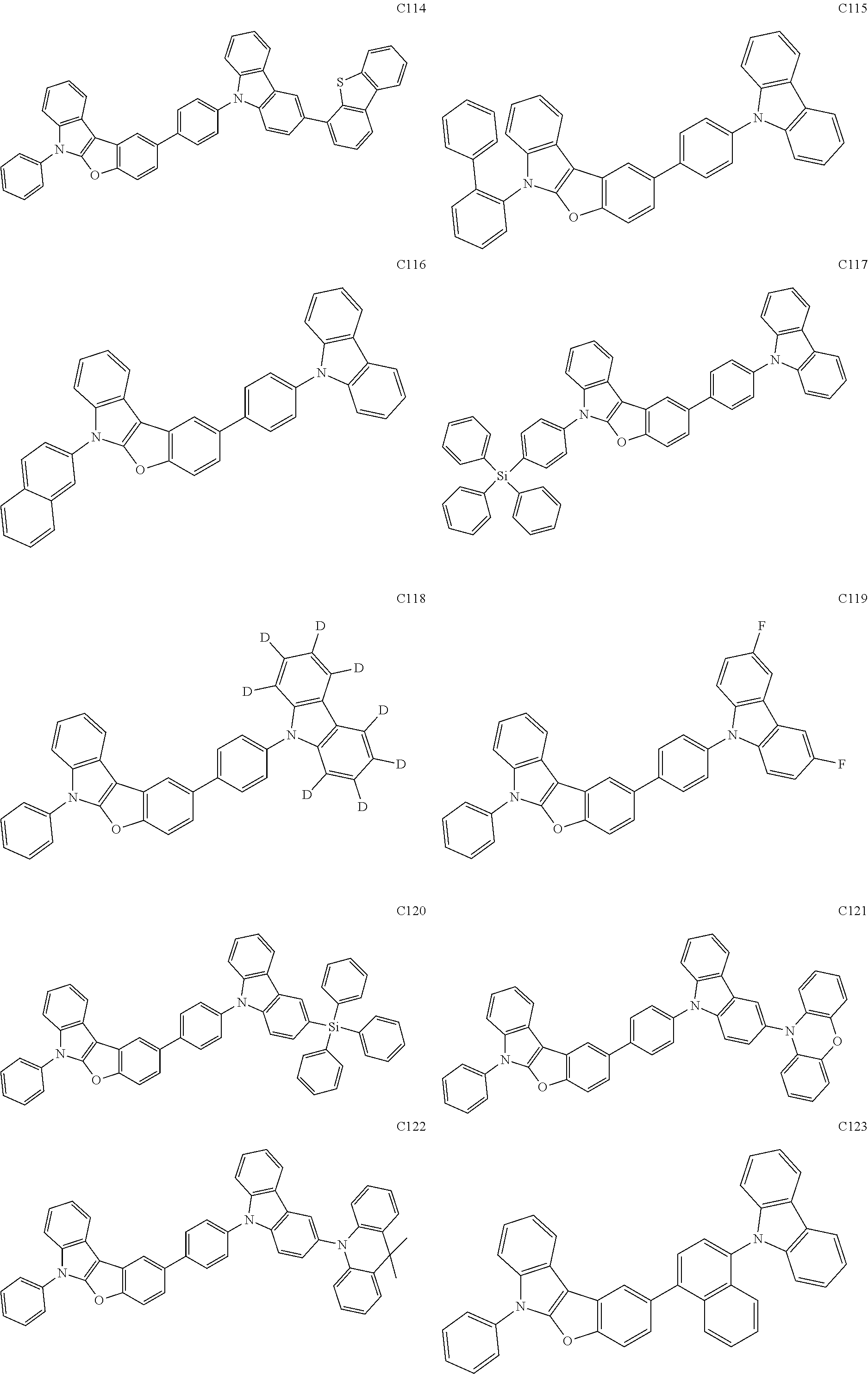

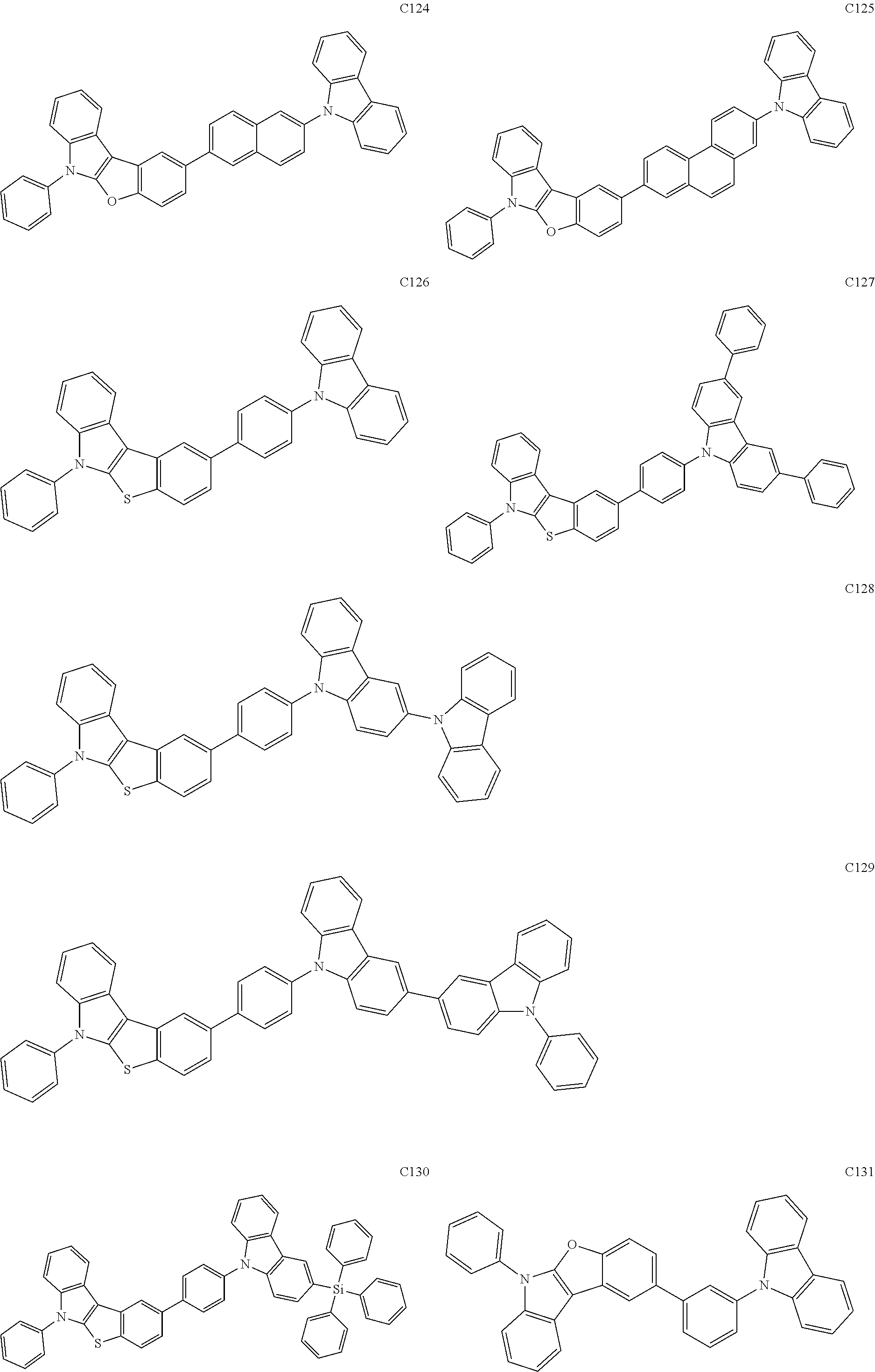

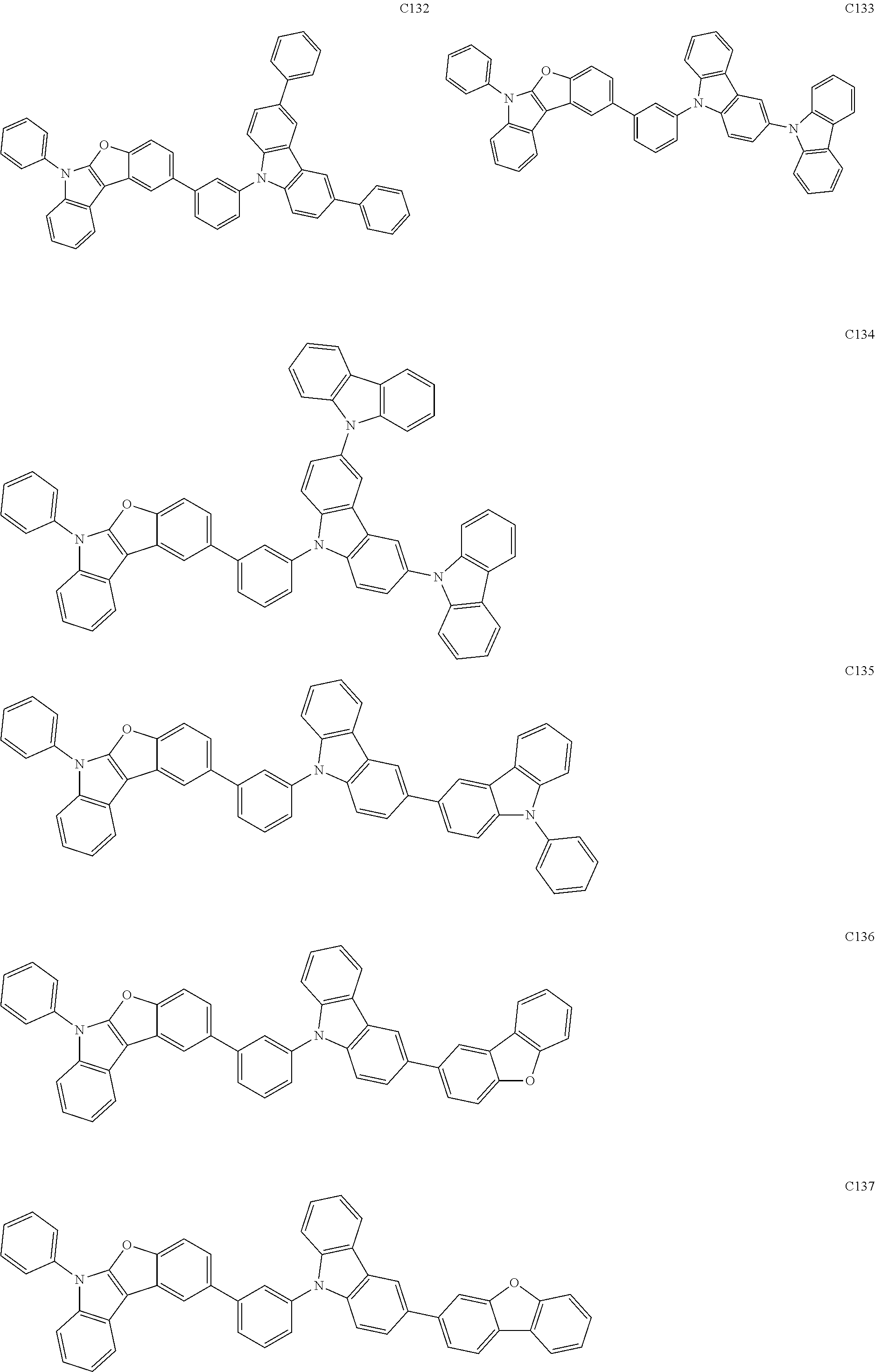

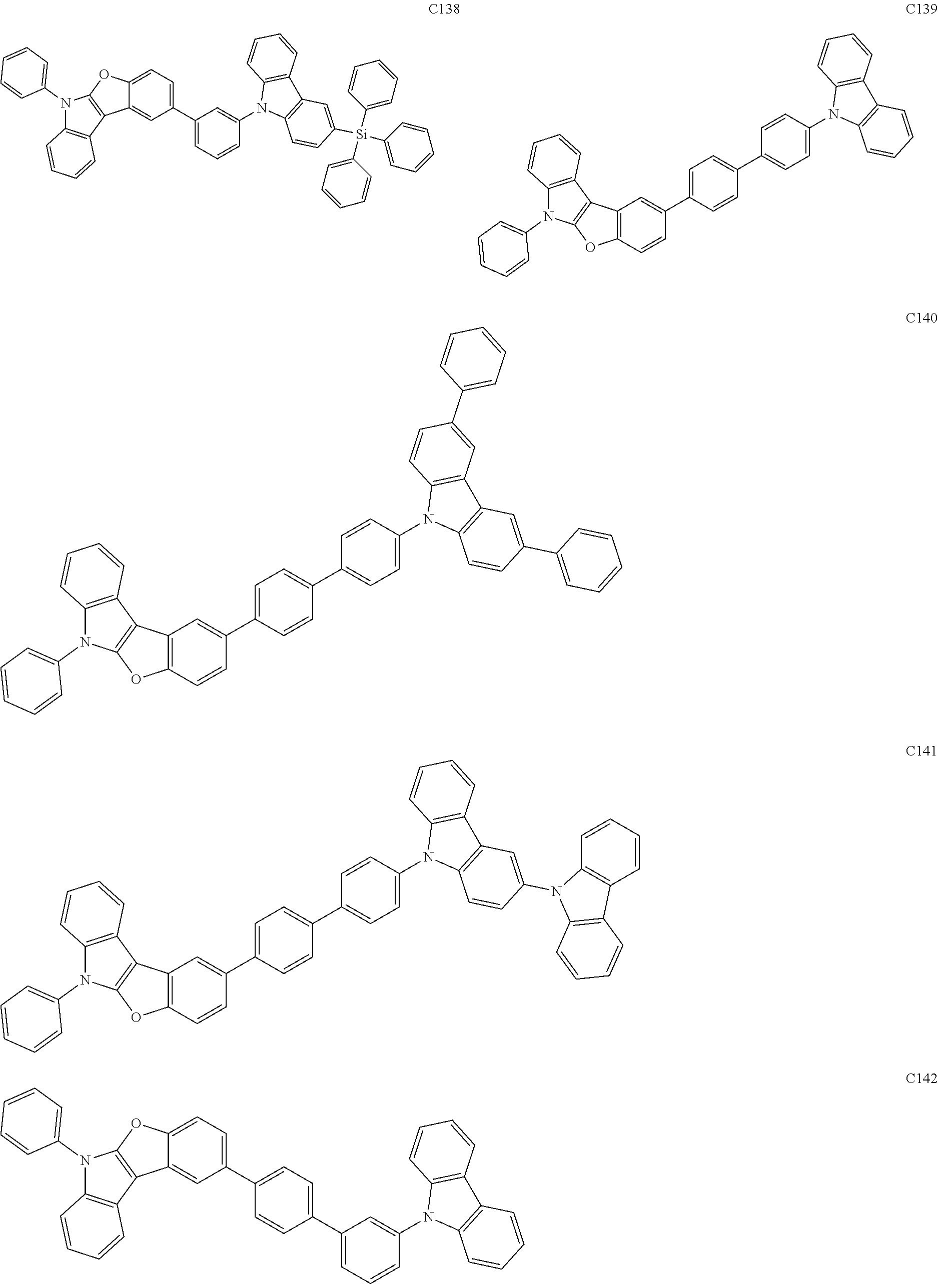

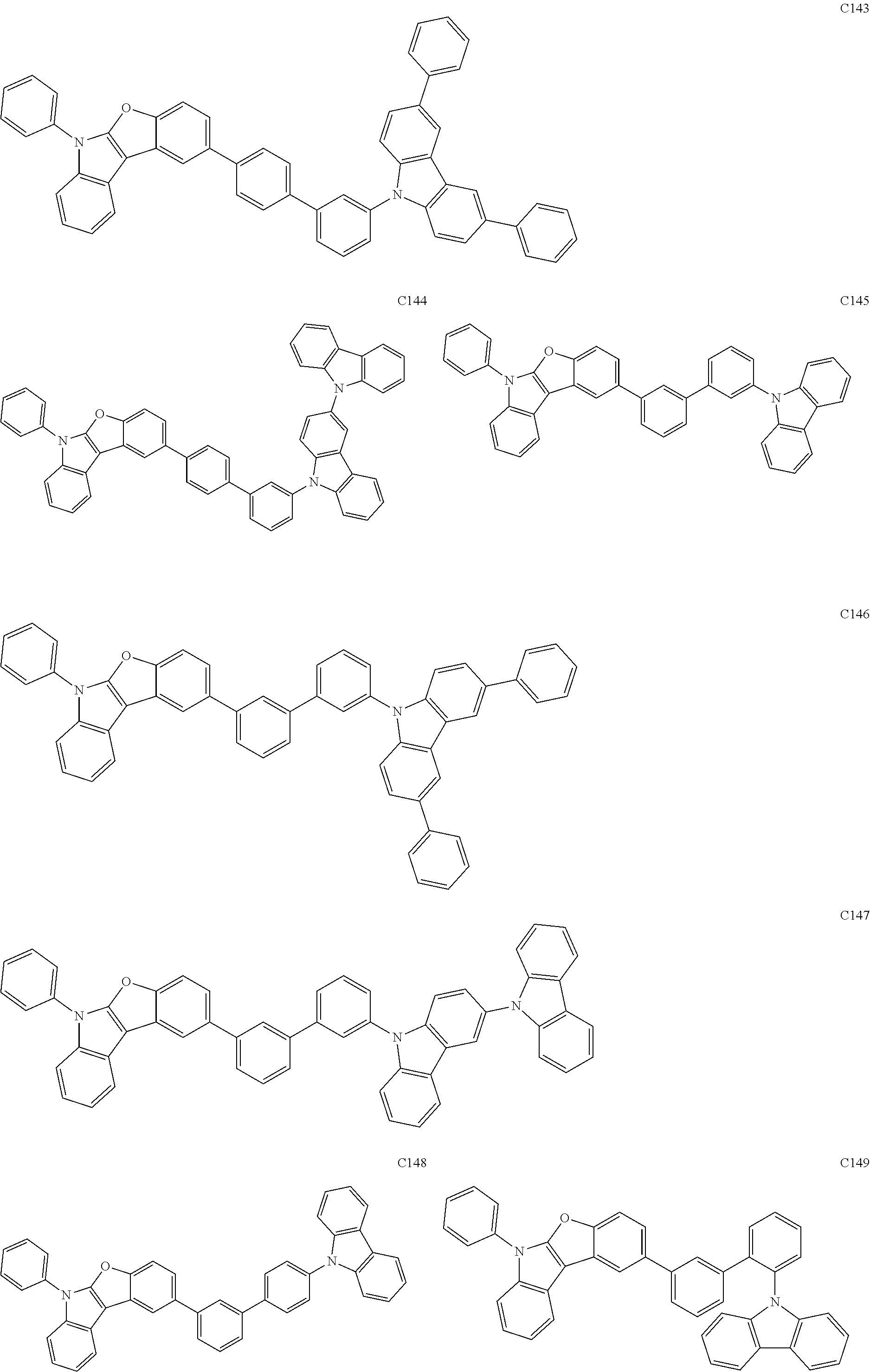

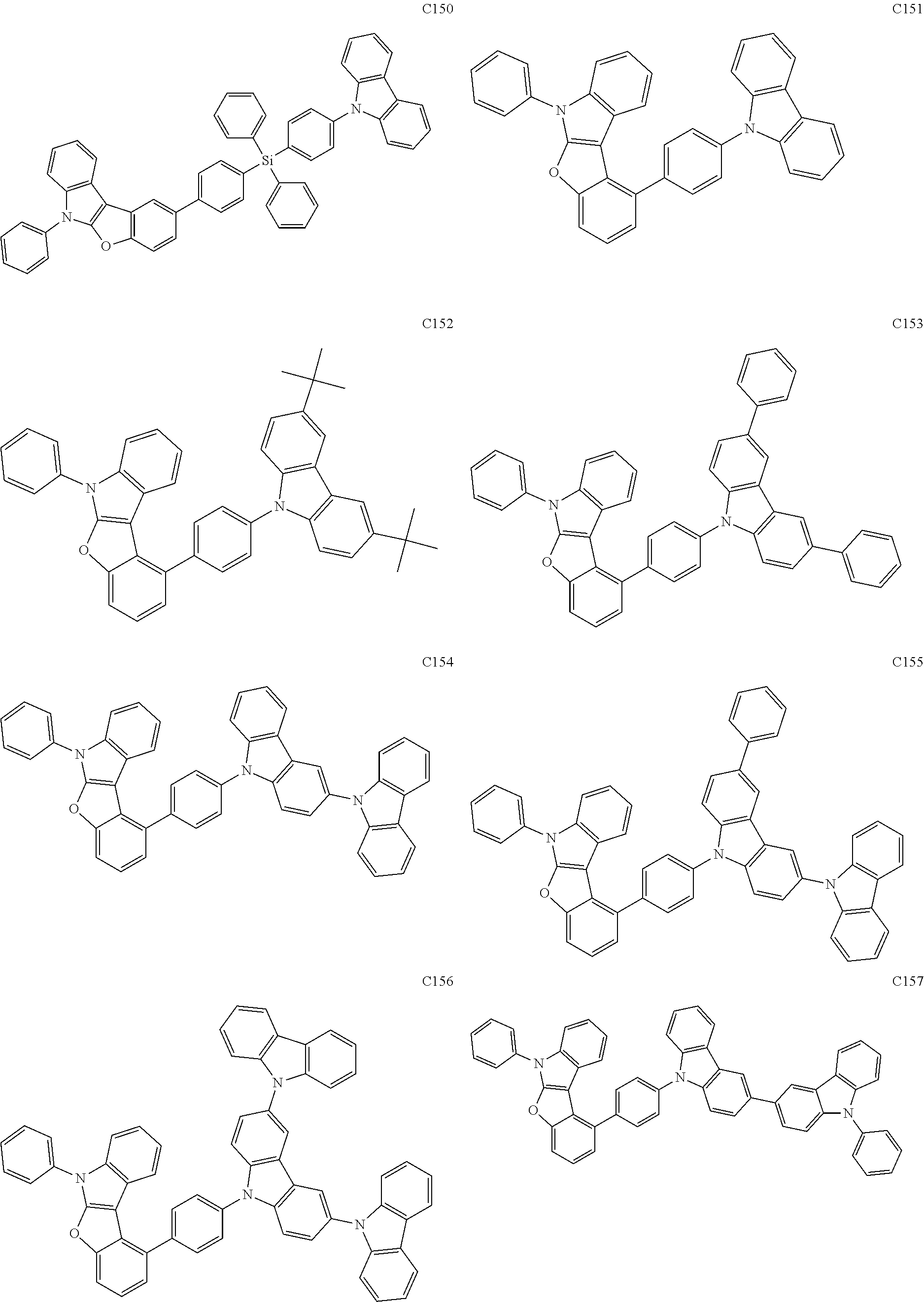

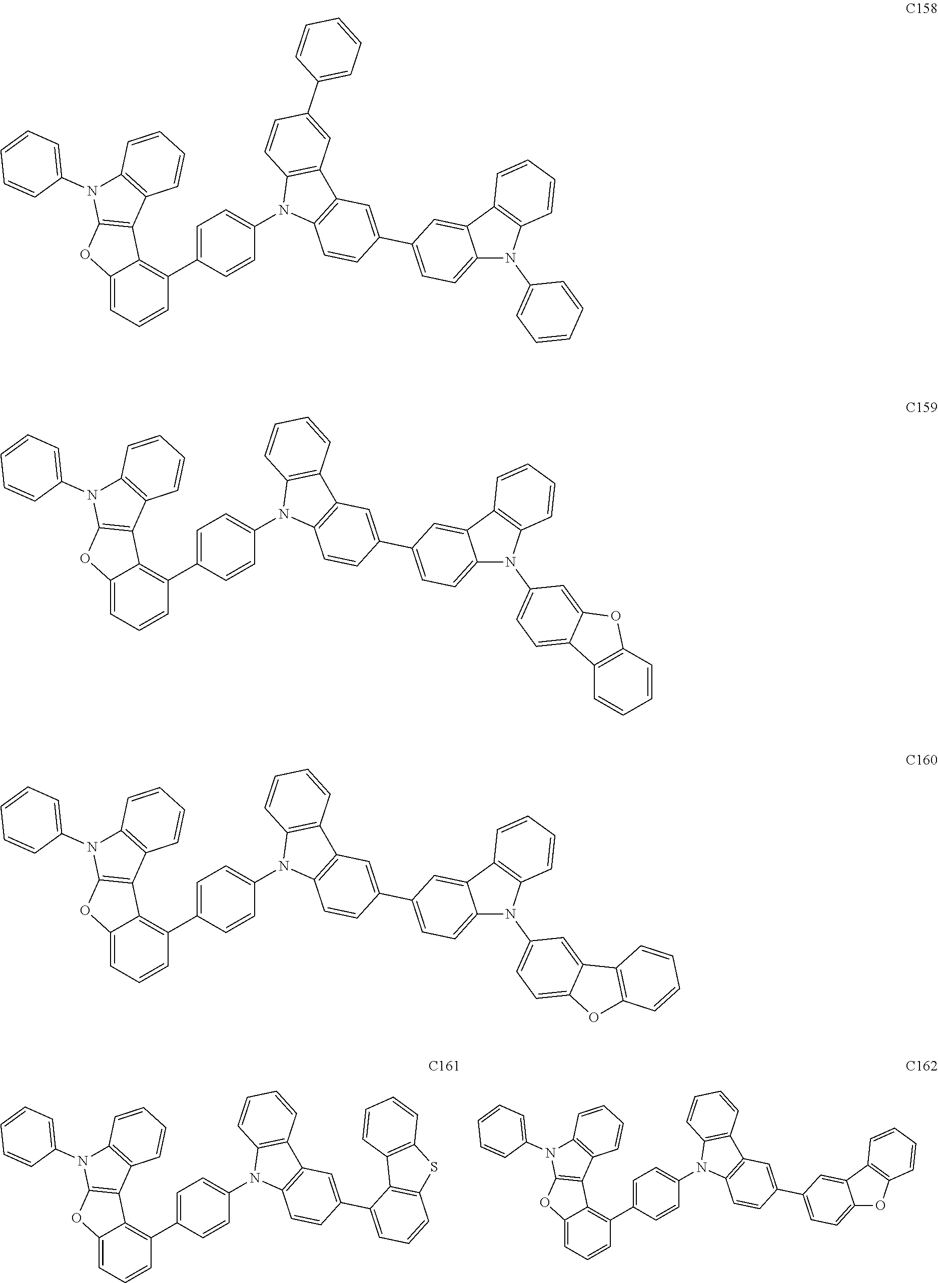

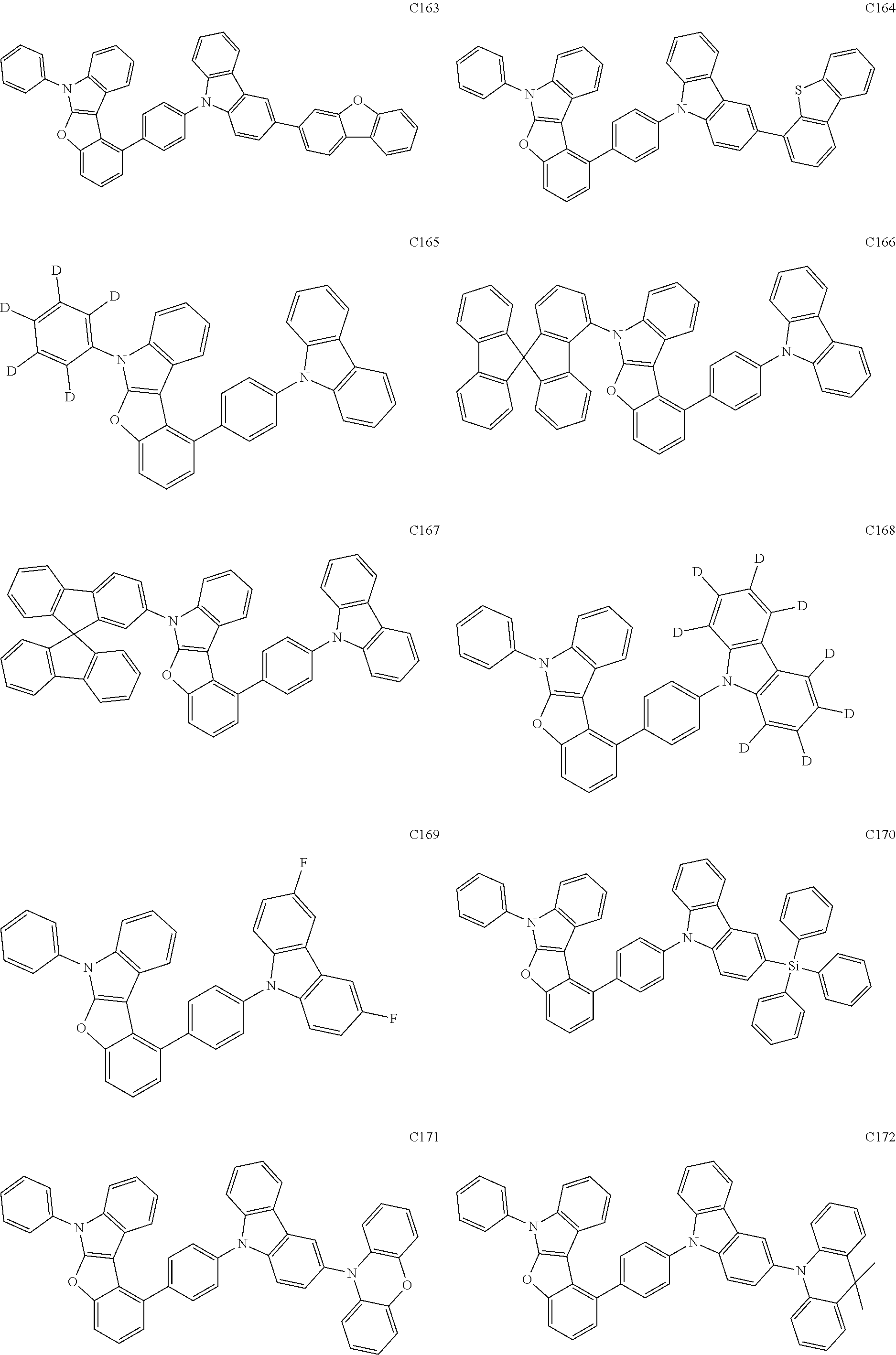

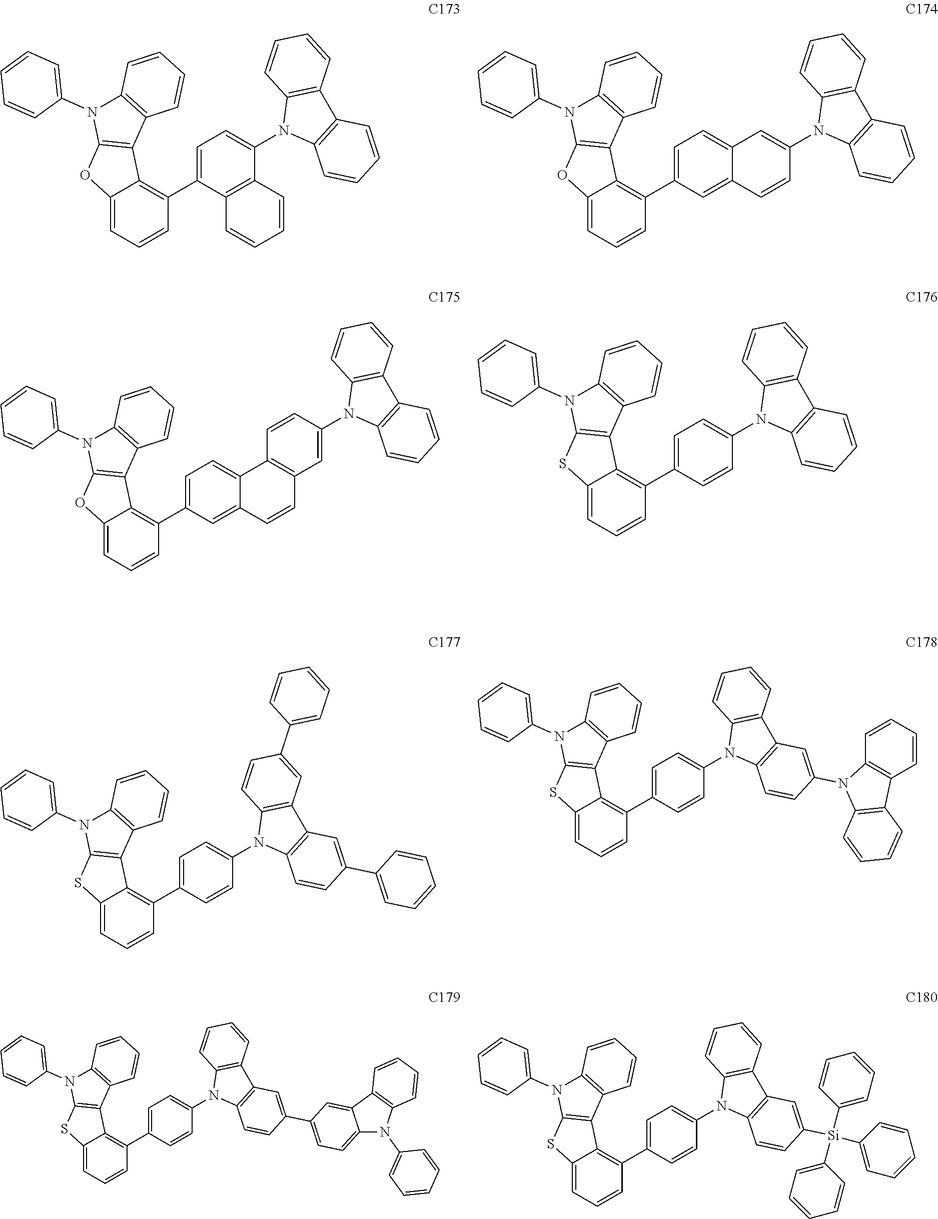

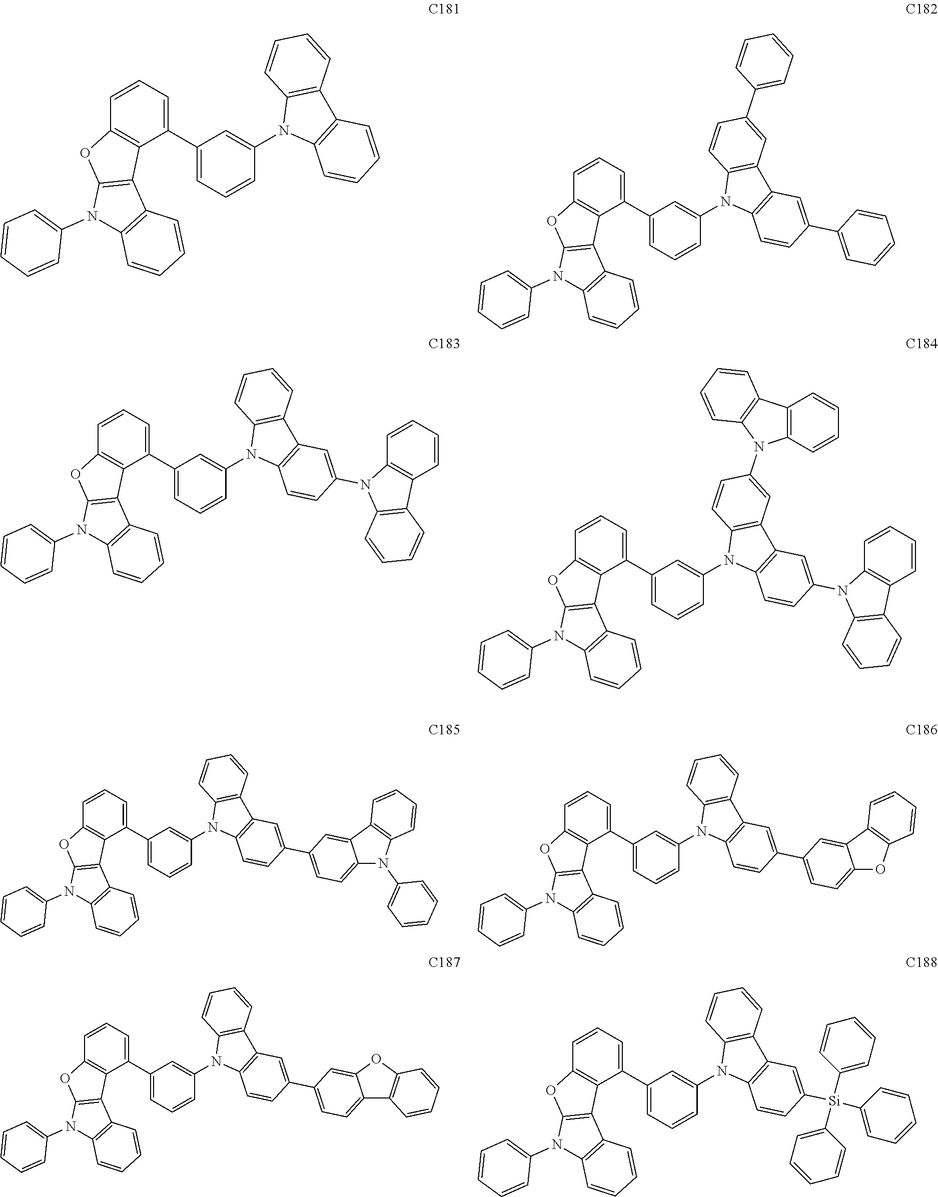

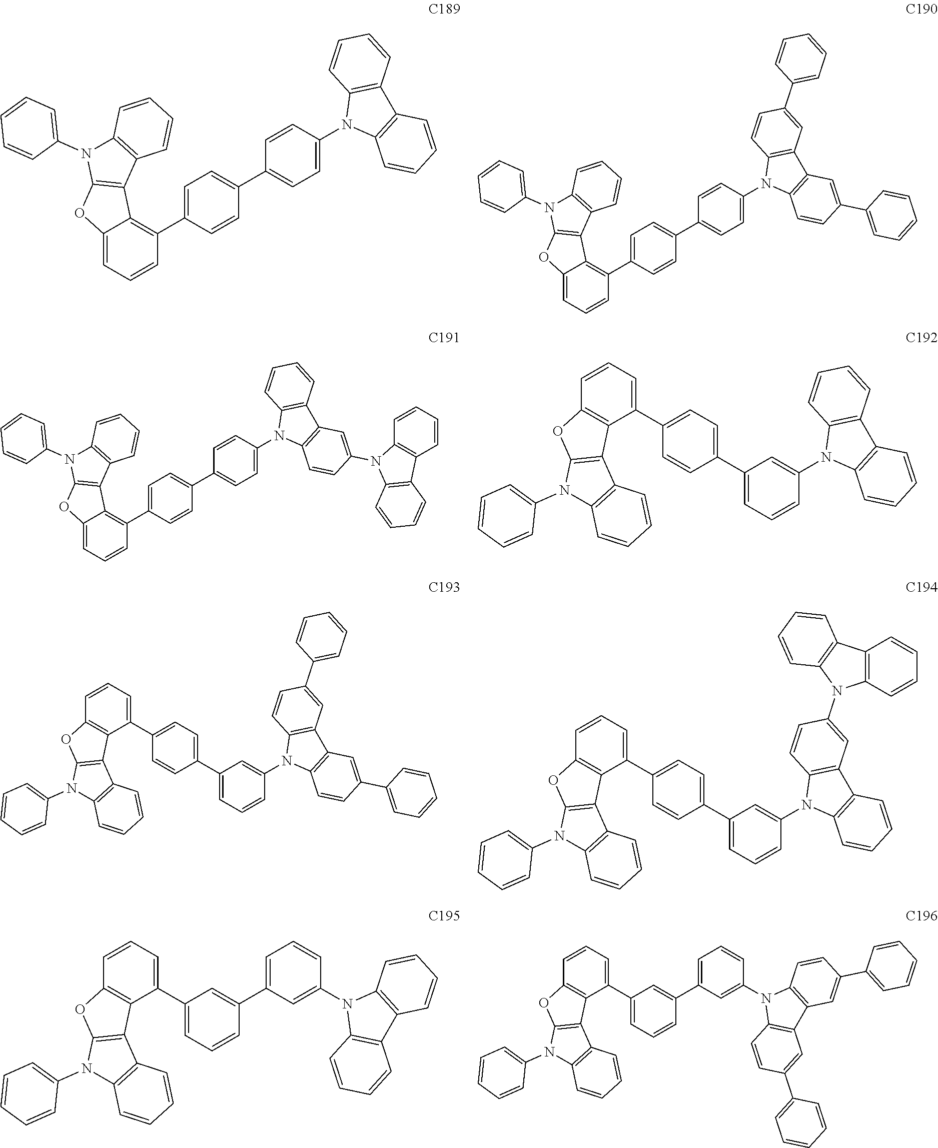

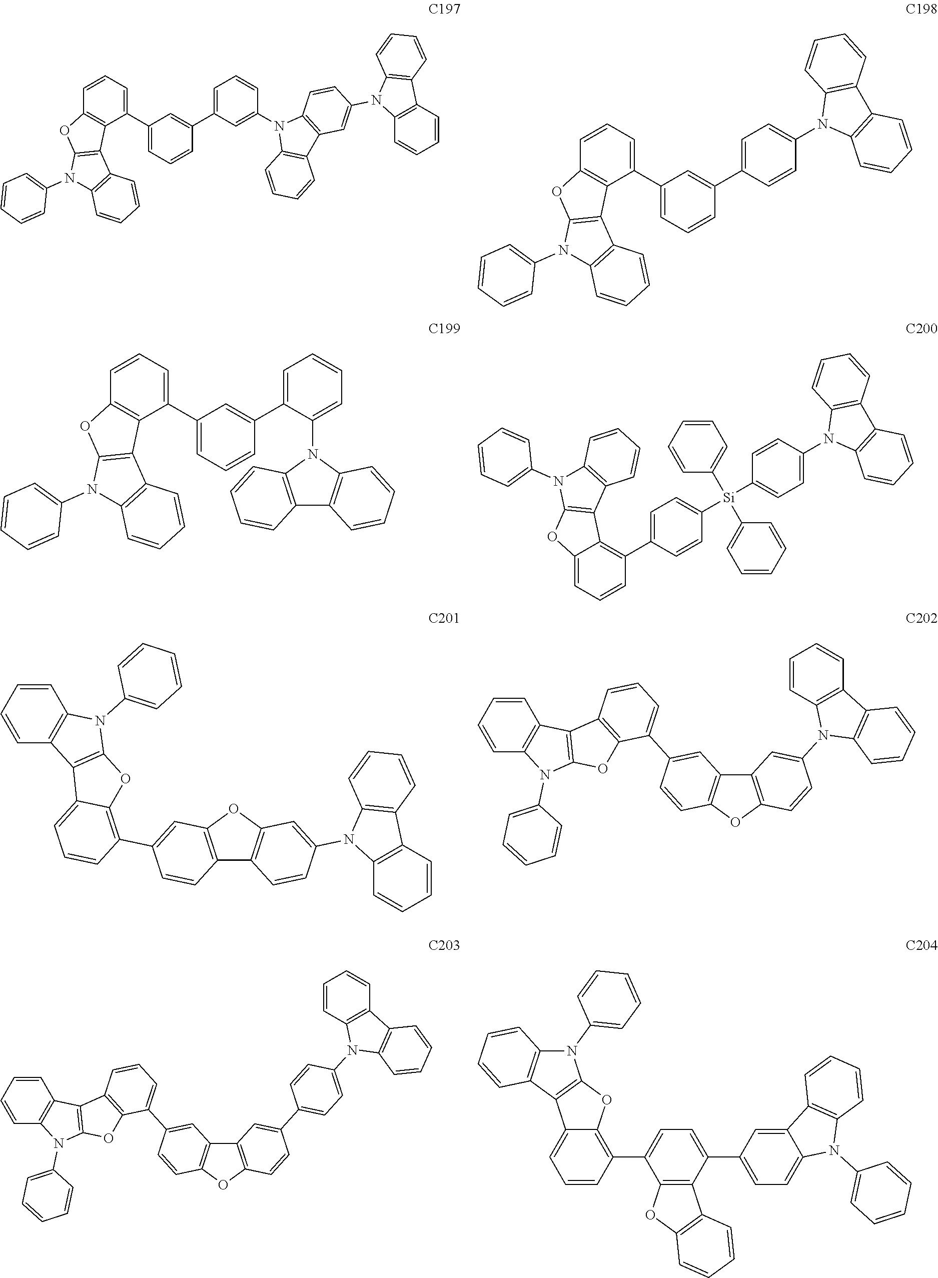

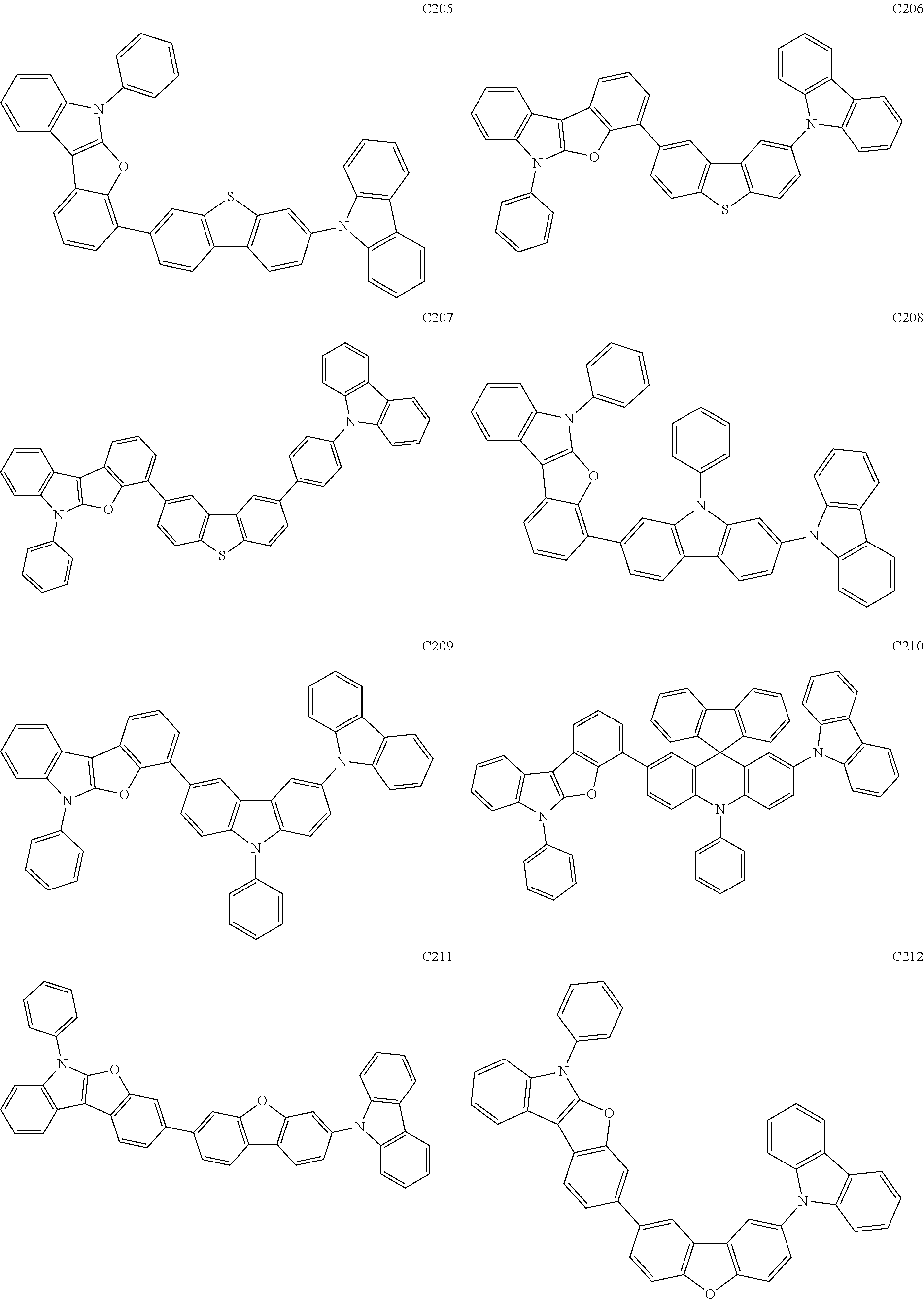

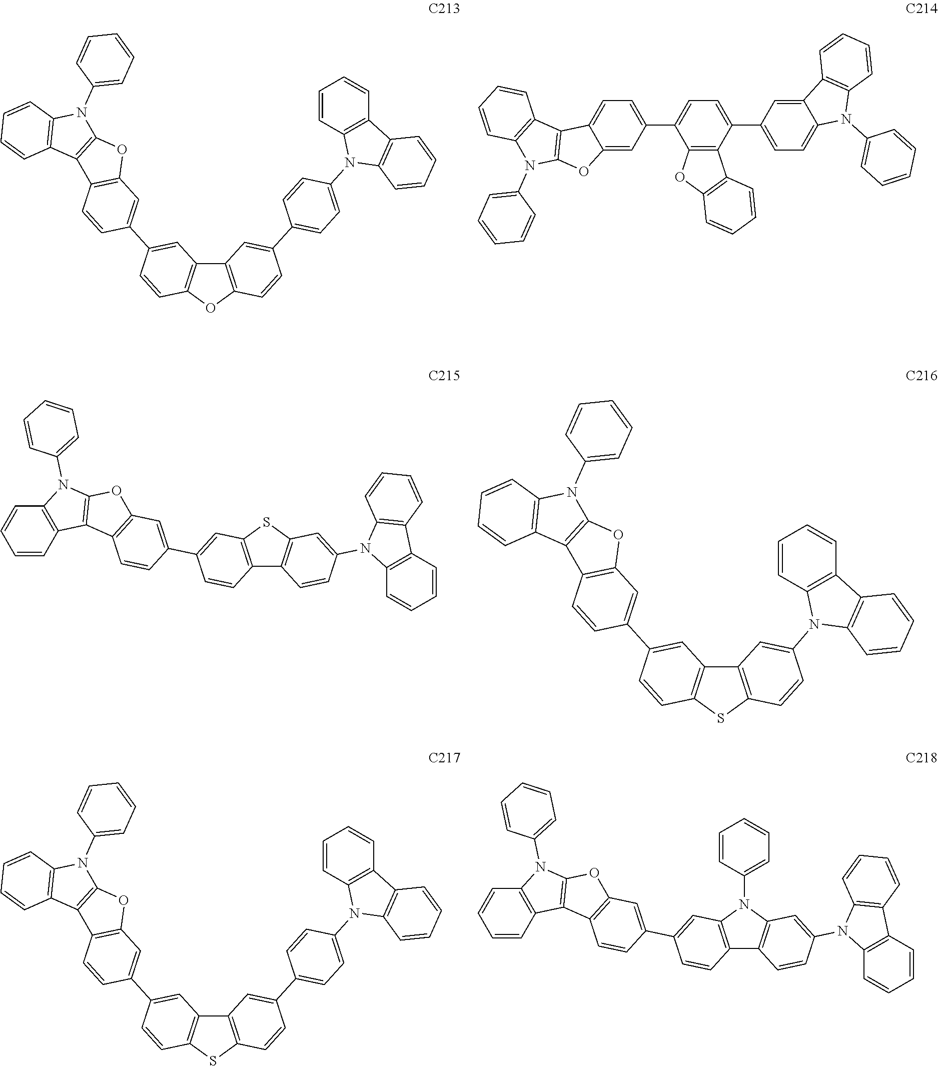

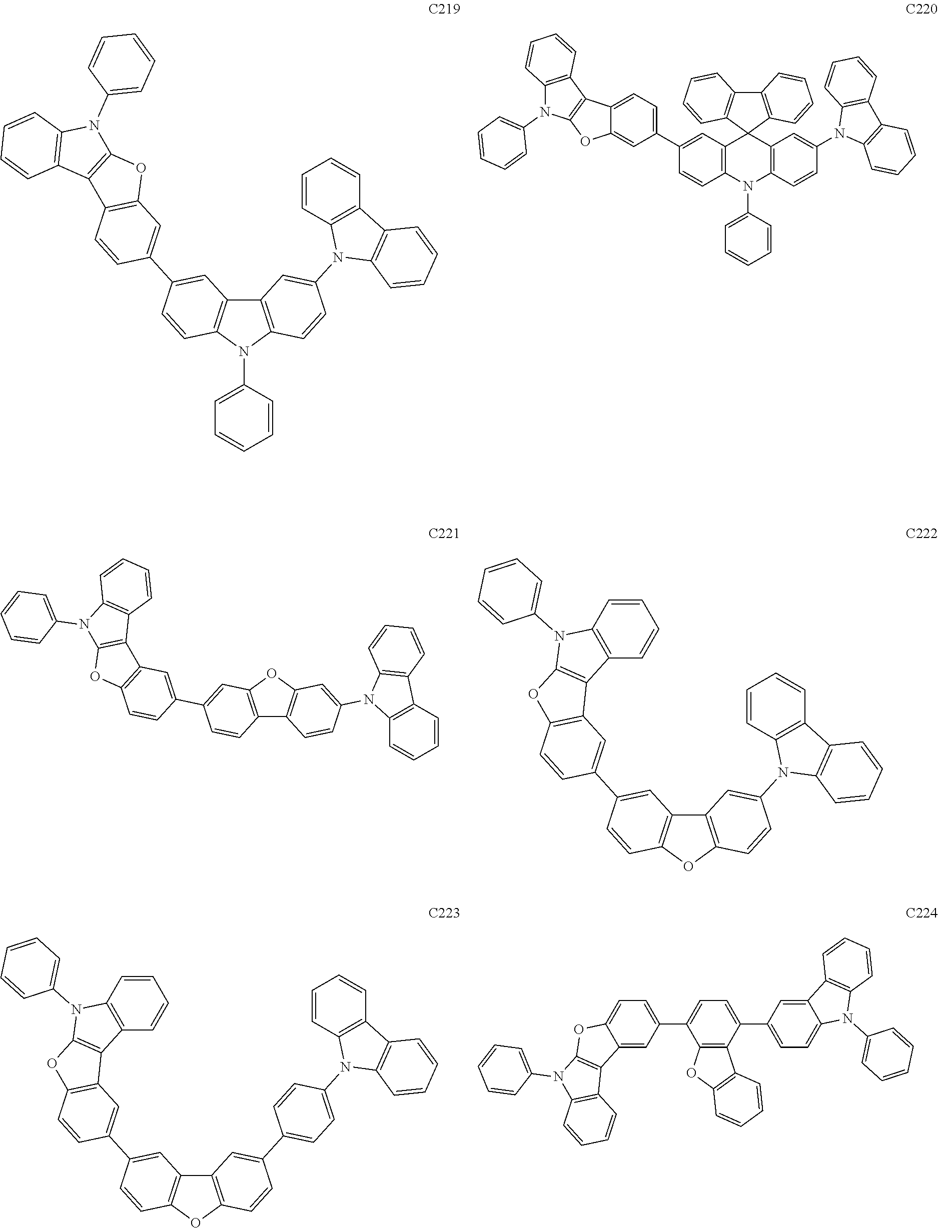

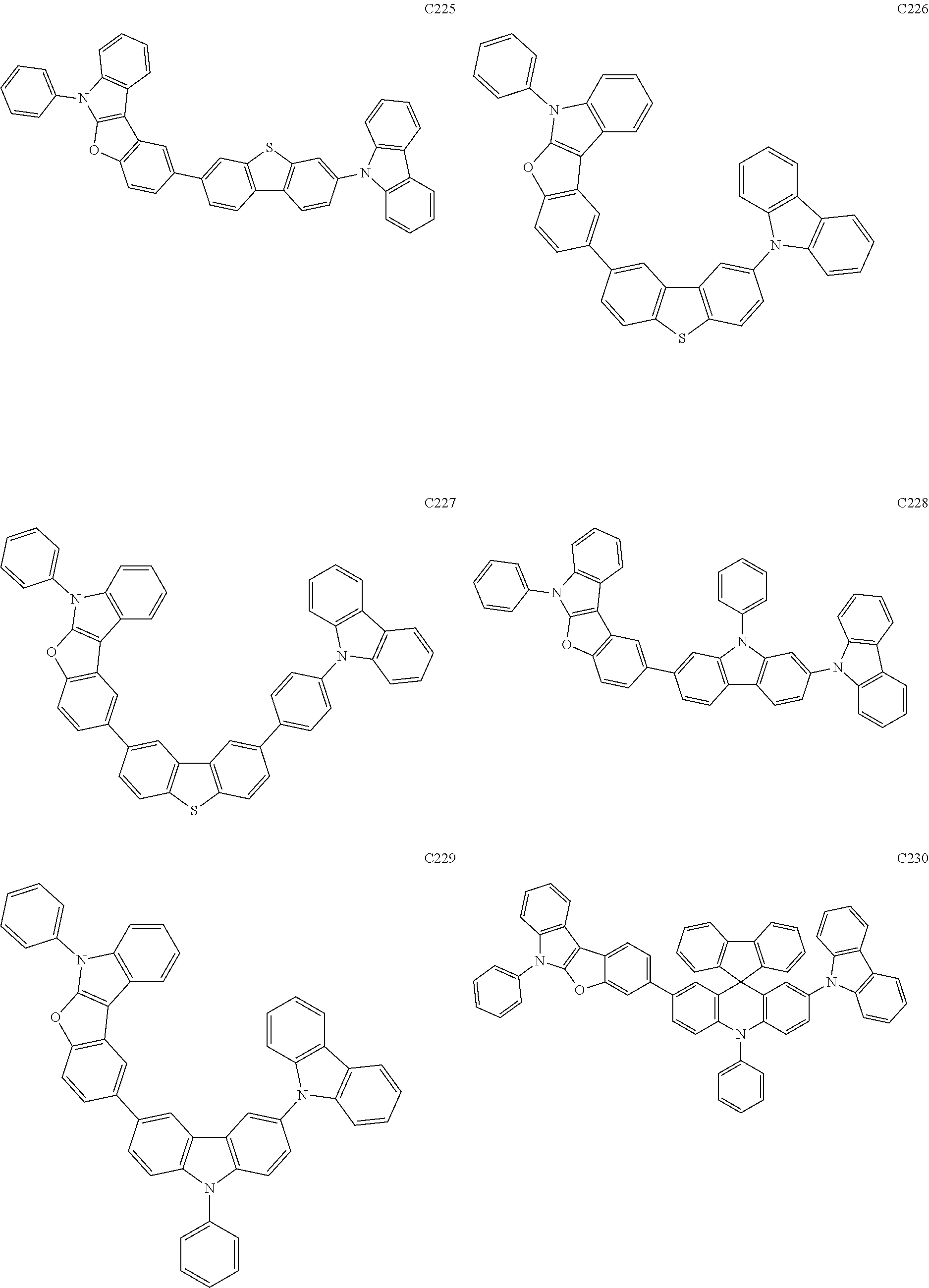

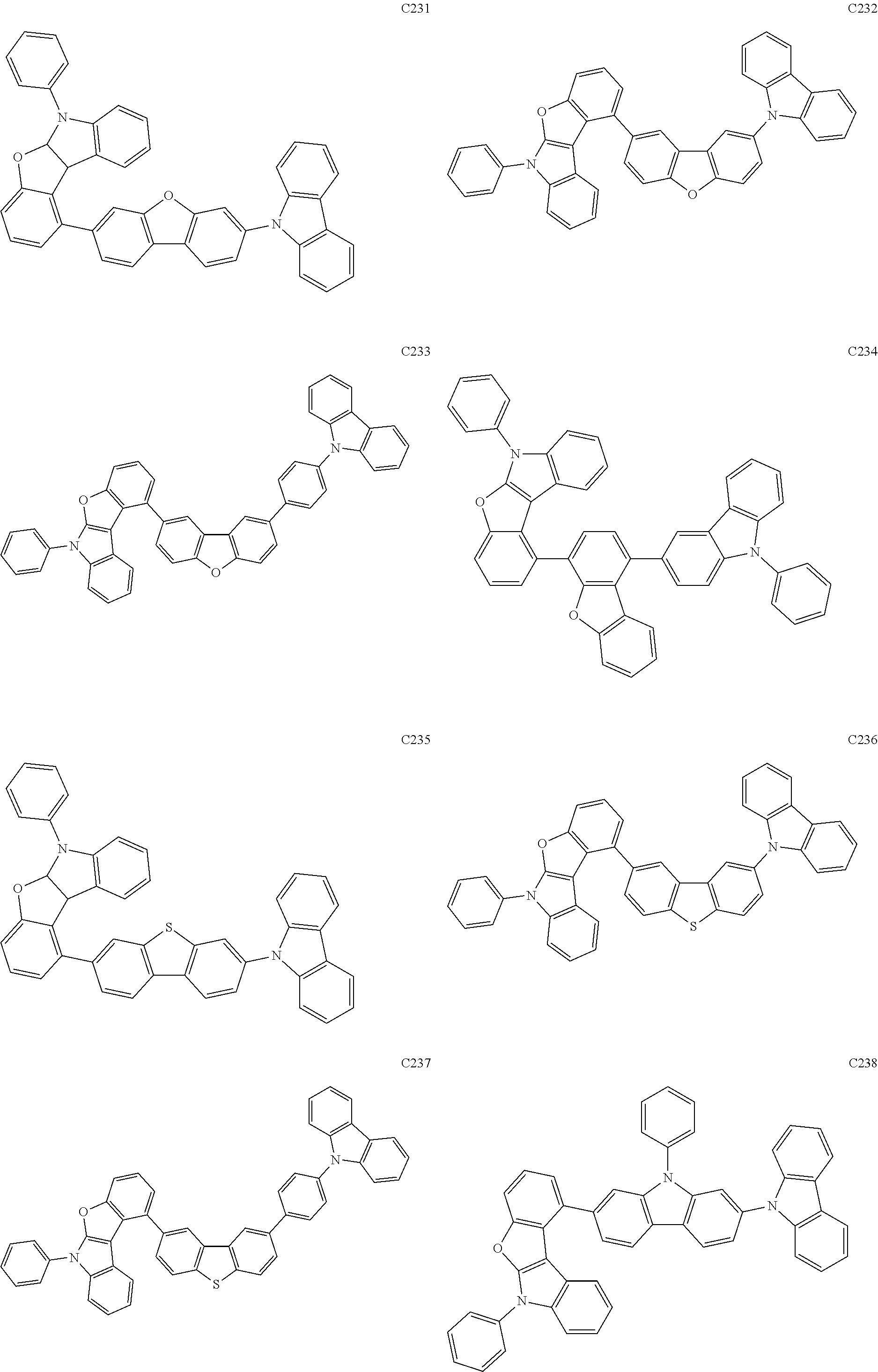

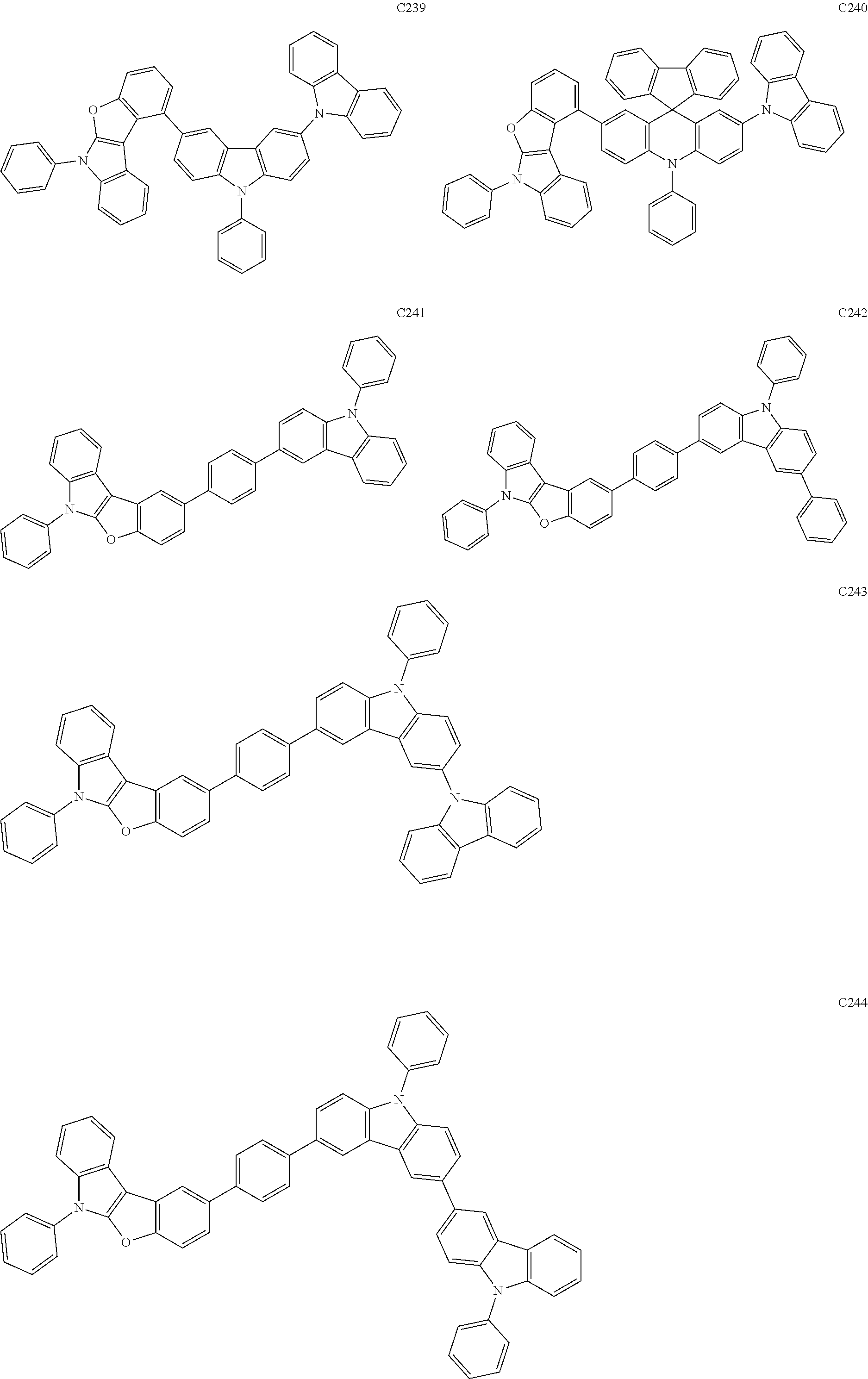

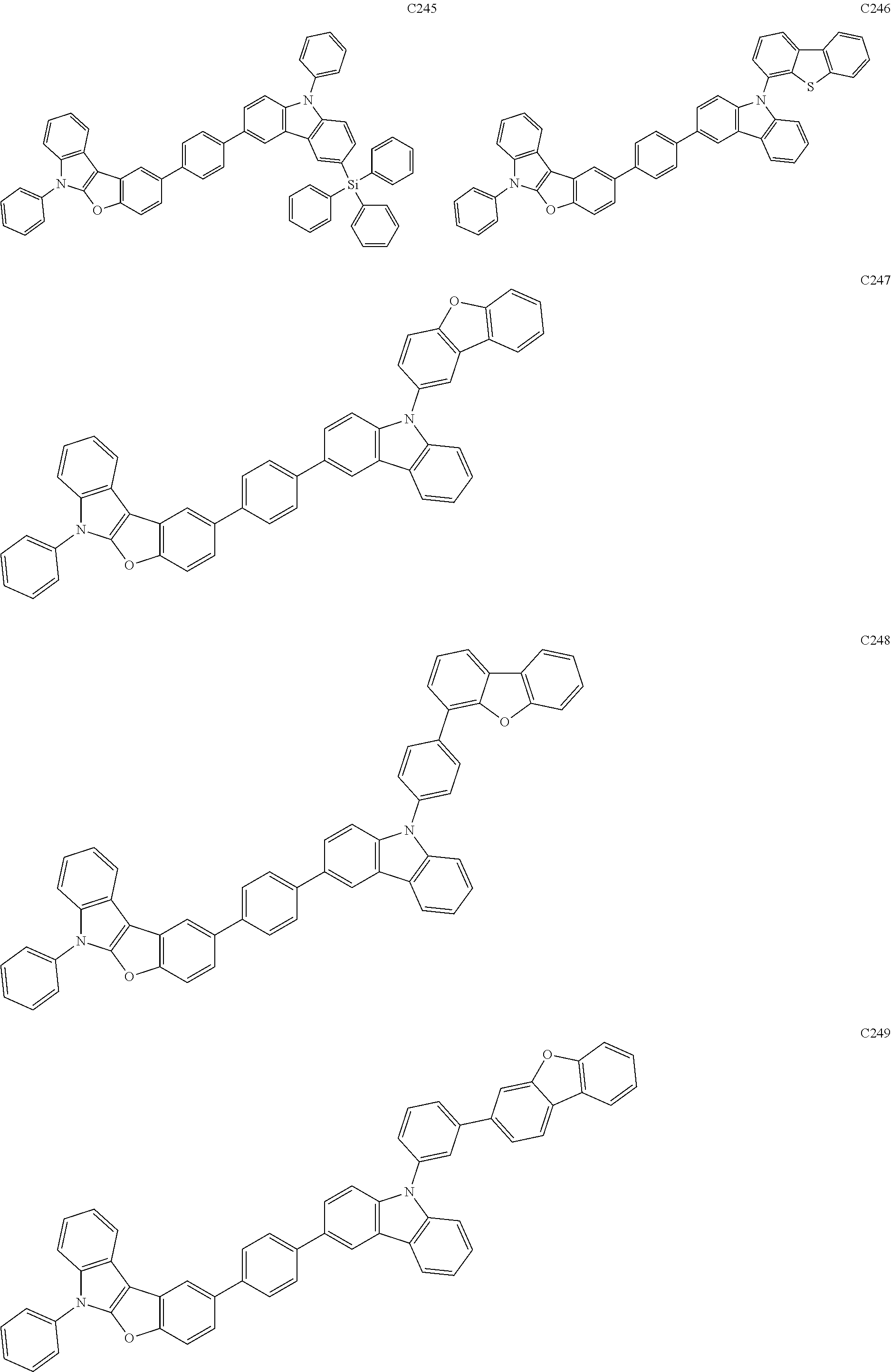

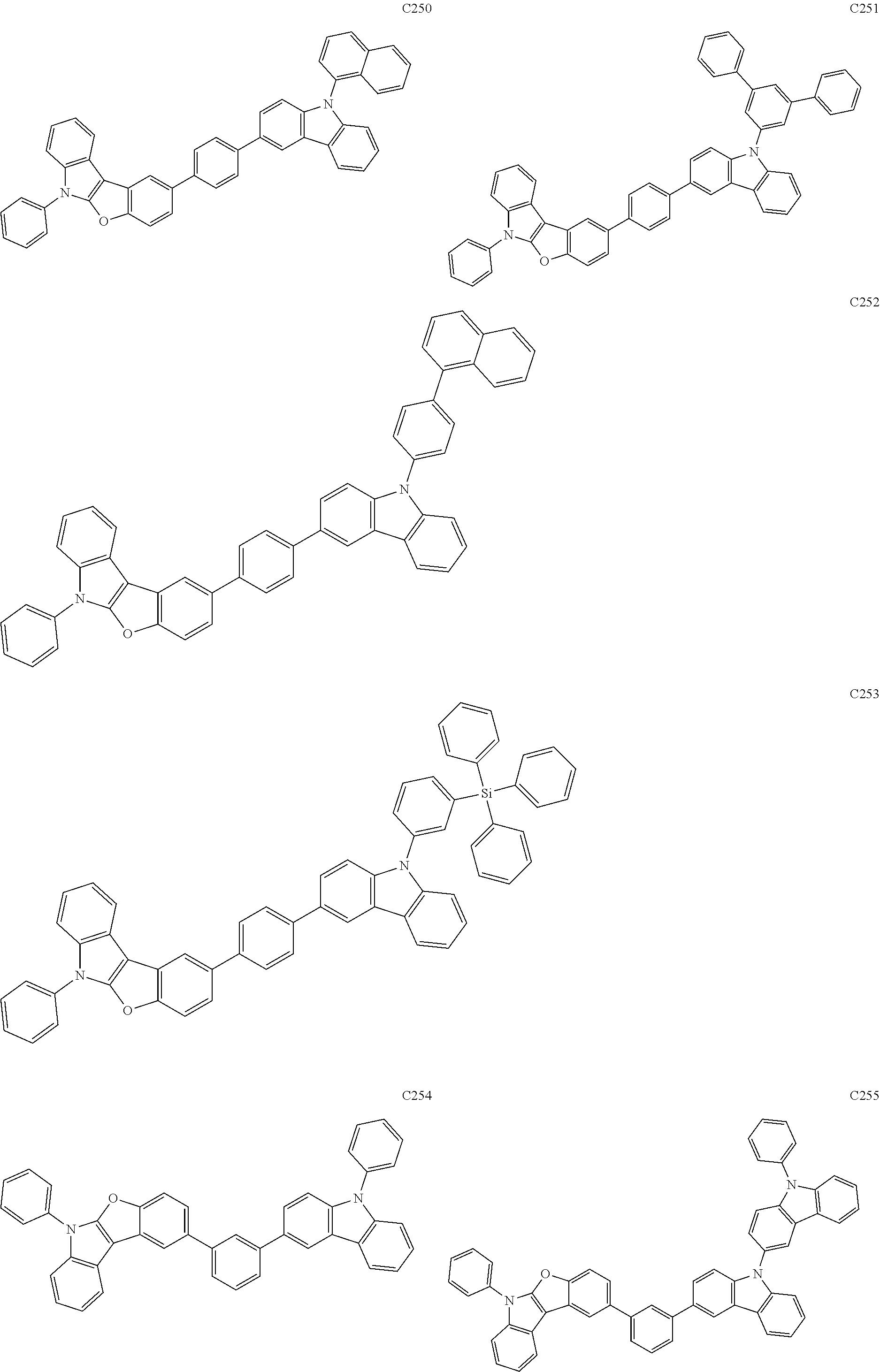

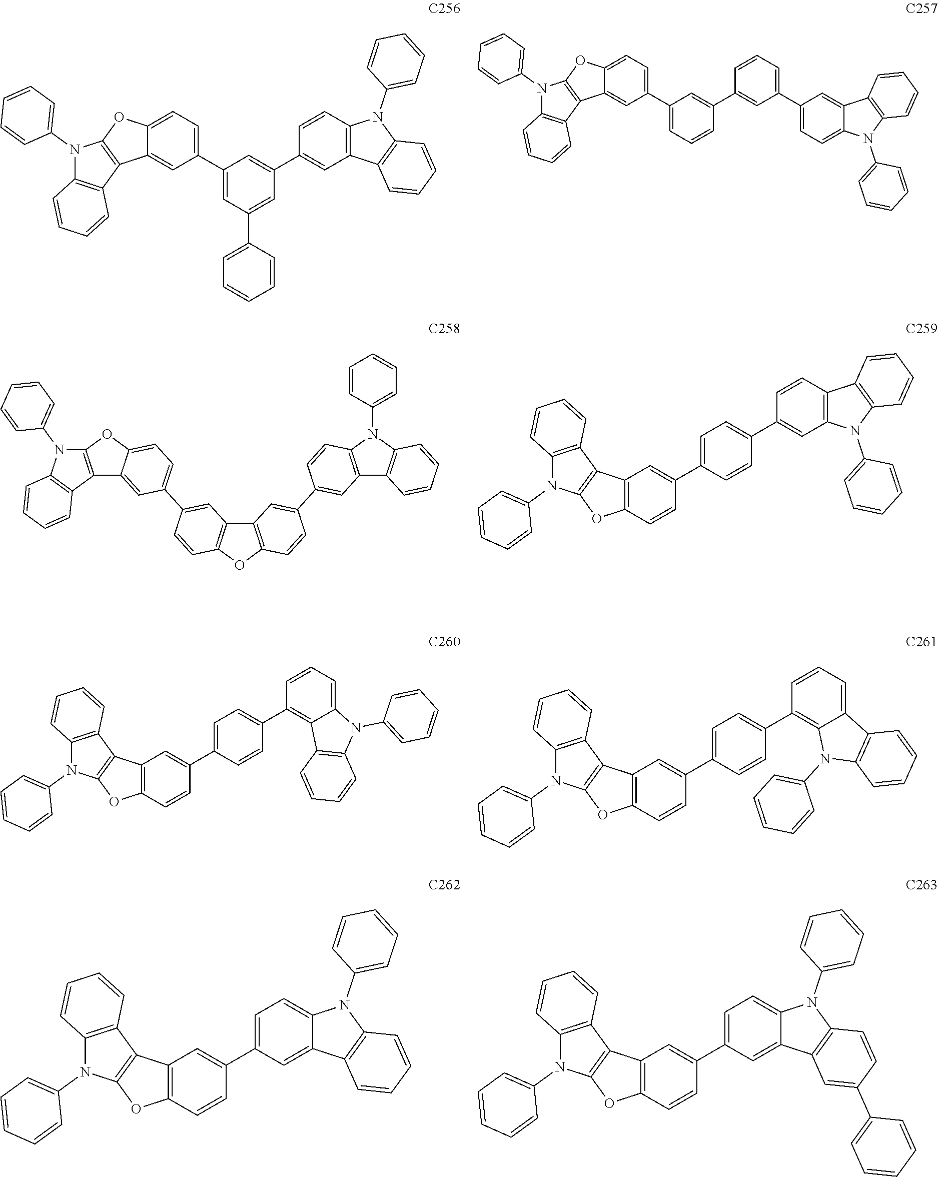

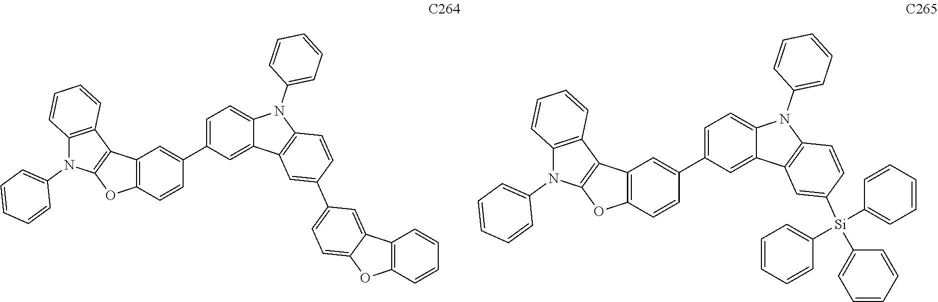 [SMILES: c1ccc(-n2c3ccc(-c4ccc5oc6ccccc6c5c4)cc3c3cc(-c4ccc5oc6c(c5c4)c4ccccc4n6-c4ccccc4)ccc32)cc1 ; c1ccc(-n2c3ccc(-c4ccc5oc6c(c5c4)c4ccccc4n6-c4ccccc4)cc3c3cc([Si](c4ccccc4)(c4ccccc4)c4ccccc4)ccc32)cc1]

15. A compound including a nitrogen moiety of Formula 1 below. 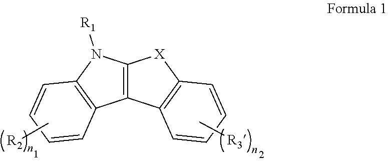
wherein, in Formula 1,
X is O or S,
R₁ to R₃ are each, independently from one another, a hydrogen atom, a deuterium atom, a halogen atom, a nitro group, a substituted or unsubstituted silyl group, a substituted or unsubstituted boron group, a substituted or unsubstituted oxy group, a substituted or unsubstituted thio group, a substituted or unsubstituted carbonyl group, a substituted or unsubstituted alkyl group having 1 to 30 carbon atoms, a substituted or unsubstituted aryl group having 6 to 60 carbon atoms, or a substituted or unsubstituted heteroaryl group 2 to 60 ring-forming carbon atoms,
n₁ and n₂ are each, independently from one another, an integer of 1 to 4, and
any one of Formula 2-1 below or Formula 2-2 below is a substituent of at least one of R₁ to R₃: 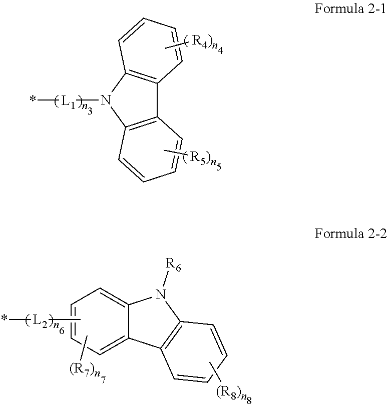
wherein, in Formula 2-1 and Formula 2-2,
L₁ and L₂ are each, independently from one another, a substituted or unsubstituted silyl linking group, a substituted or unsubstituted arylene group having 6 to 60 ring-forming carbon atoms, or a substituted or unsubstituted heteroarylene group having 2 to 60 ring-forming carbon atoms, excluding when each of L₁ and L₂ is a 2,2′-biphenyl linking group, a pyridine linking group, a pyrazine linking group, a quinoline linking group, a quinoxaline linking group, and a triazine linking group,
R₄ to R₈ are each, independently from one another, a hydrogen atom, a deuterium atom, a halogen atom, a nitro group, a substituted or unsubstituted silyl group, a substituted or unsubstituted boron group, a substituted or unsubstituted oxy group, a substituted or unsubstituted thio group, a substituted or unsubstituted carbonyl group, a substituted or unsubstituted alkyl group having 1 to 30 carbon atoms, a substituted or unsubstituted aryl group having 6 to 60 ring-forming carbon atoms, or a substituted or unsubstituted heteroaryl group having 2 to 60 ring-forming carbon atoms,
n₃ and n₇ are each, independently from one another, an integer of 1 to 3,
n₄, n₅, and n₈ are each, independently from one another, an integer of 1 to 4,
n₆ is an integer of 0 to 3, [symbol]
is a position linked to Formula 1,
when a substituent of Formula 2-1 is substituted in the compound of Formula 1, L₁ is a substituted or unsubstituted arylene group having 6 to 60 ring-forming carbon atoms, and n₃ is 1,
at least one of R₂ to R₅ is a substituted or unsubstituted silyl group, a substituted or unsubstituted aryl group having 6 to 60 ring-forming carbon atoms, or a substituted or unsubstituted heteroaryl group having 2 to 60 ring-forming carbon atoms.

16. The compound of claim 15, wherein the compound of Formula 1 is of any one of Formula 1-1 to Formula 1-3 below: 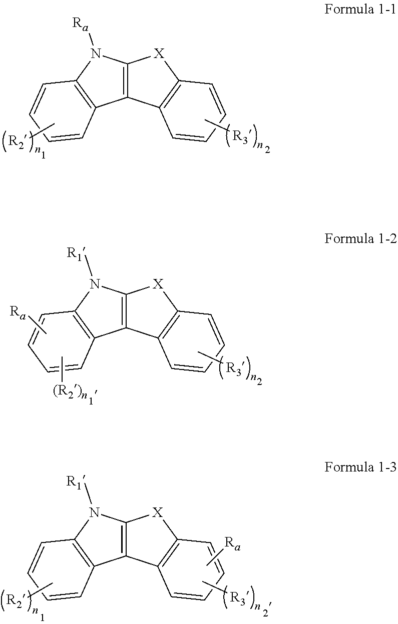
wherein, in Formula 1-1 to Formula 1-3,
Rₐ is a substituent of Formula 2-1 or Formula 2-2,
R₁′, R₂′, and R₃′ are each, independently from one another, a hydrogen atom, a deuterium atom, a halogen atom, a nitro group, a substituted or unsubstituted silyl group, a substituted or unsubstituted boron group, a substituted or unsubstituted oxy group, a substituted or unsubstituted thio group, a substituted or unsubstituted carbonyl group, a substituted or unsubstituted alkyl group having 1 to 30 carbon atoms, a substituted or unsubstituted aryl group having 6 to 60 ring-forming carbon atoms, or a substituted or unsubstituted heteroaryl group having 2 to 60 ring-forming carbon atoms, excluding when each of R₁′, R₂′, and R₃′ is of Formula 2-1 or Formula 2-2,
n₁′ and n₂′ are each, independently from one another, an integer of 1 to 3, and
X, n₁ and n₂ have, independently from one another, the same meaning as in Formula 1.

17. The compound of claim 15, wherein the substituent of Formula 2-1 is of Formula 2-1-1 below: 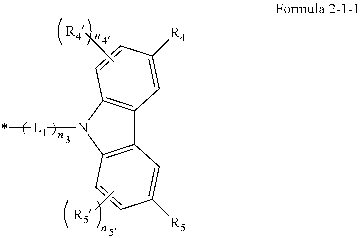
wherein, in Formula 2-1-1,
R₄′ and R₅′ are each, independently from one another, a hydrogen atom or a deuterium atom,
n₄′ and n₅′ are each, independently from one another, an integer of 1 to 3, and
L₁, n₃, R₄, and R₅ have, independently from one another, the same meaning as in Formula 2-1.

18. The compound of claim 15, wherein the substituent of Formula 2-2 is any one of Formula 2-2-1 to Formula 2-2-4 below: 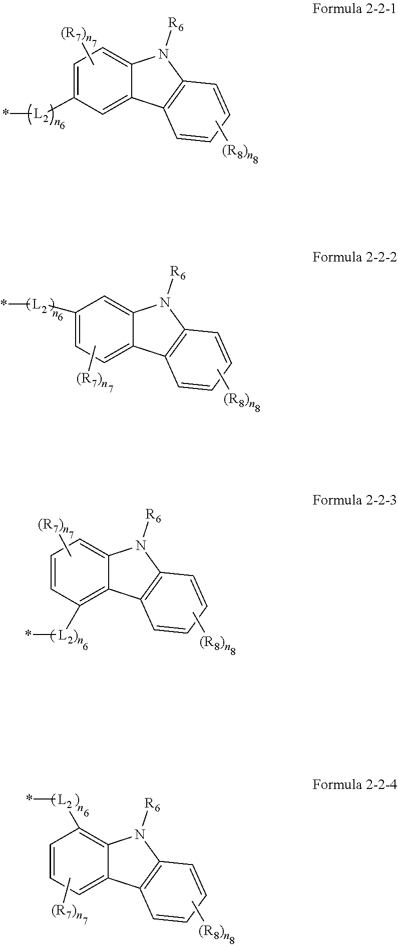
wherein, in Formula 2-2-1 and Formula 2-2-4,
L₂, R₆ to R₈, and n₆ to n₈ have, independently from one another, the same meaning as in in Formula 2-2 in claim 15.

19. The compound of claim 15, wherein L₁ and L₂ are each, independently from one another, any one of Formula 3-1 to Formula 3-12 below: 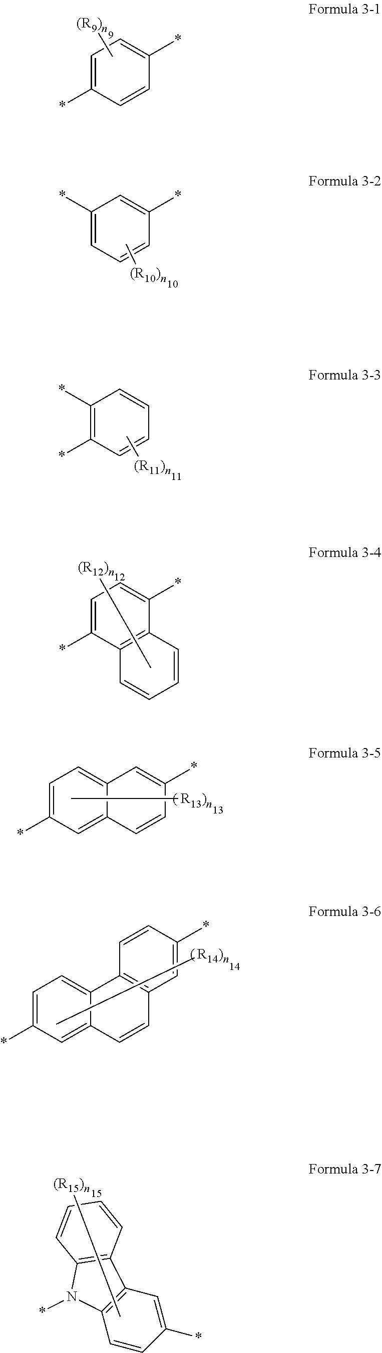 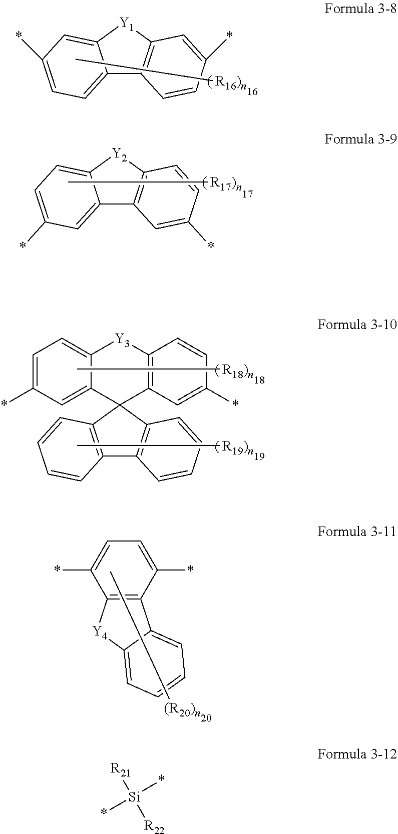
wherein, in Formula 3-1 to Formula 3-12,
Y₁ to Y₄ are each, independently from one another, NR_{b}, O or S,
R_{b} and R₉ to R₂₂ are each, independently from one another, a hydrogen atom, a deuterium atom, a halogen atom, a nitro group, a substituted or unsubstituted silyl group, a substituted or unsubstituted boron group, a substituted or unsubstituted oxy group, a substituted or unsubstituted thio group, a substituted or unsubstituted carbonyl group, a substituted or unsubstituted alkyl group having 1 to 30 carbon atoms, a substituted or unsubstituted aryl group having 6 to 60 carbon atoms, or a substituted or unsubstituted heteroaryl group 2 to 60 ring-forming carbon atoms,
n₉ to n₁₁ are each, independently from one another, an integer of 1 to 4,
n₁₂, n₁₃, n₁₅ to n₁₈, and n₂₀ are each, independently from one another, an integer of 1 to 6,
n₁₄ and n₁₉ are each, independently from one another, an integer of 1 to 8, and [symbol]
is a position linked to Formula 1 and a carbazole moiety of Formula 2-1 and Formula 2-2.

20. The compound of claim 15, wherein the compound of Formula 1 comprises at least one compound of Compound Group 1 to Compound Group 3 below: 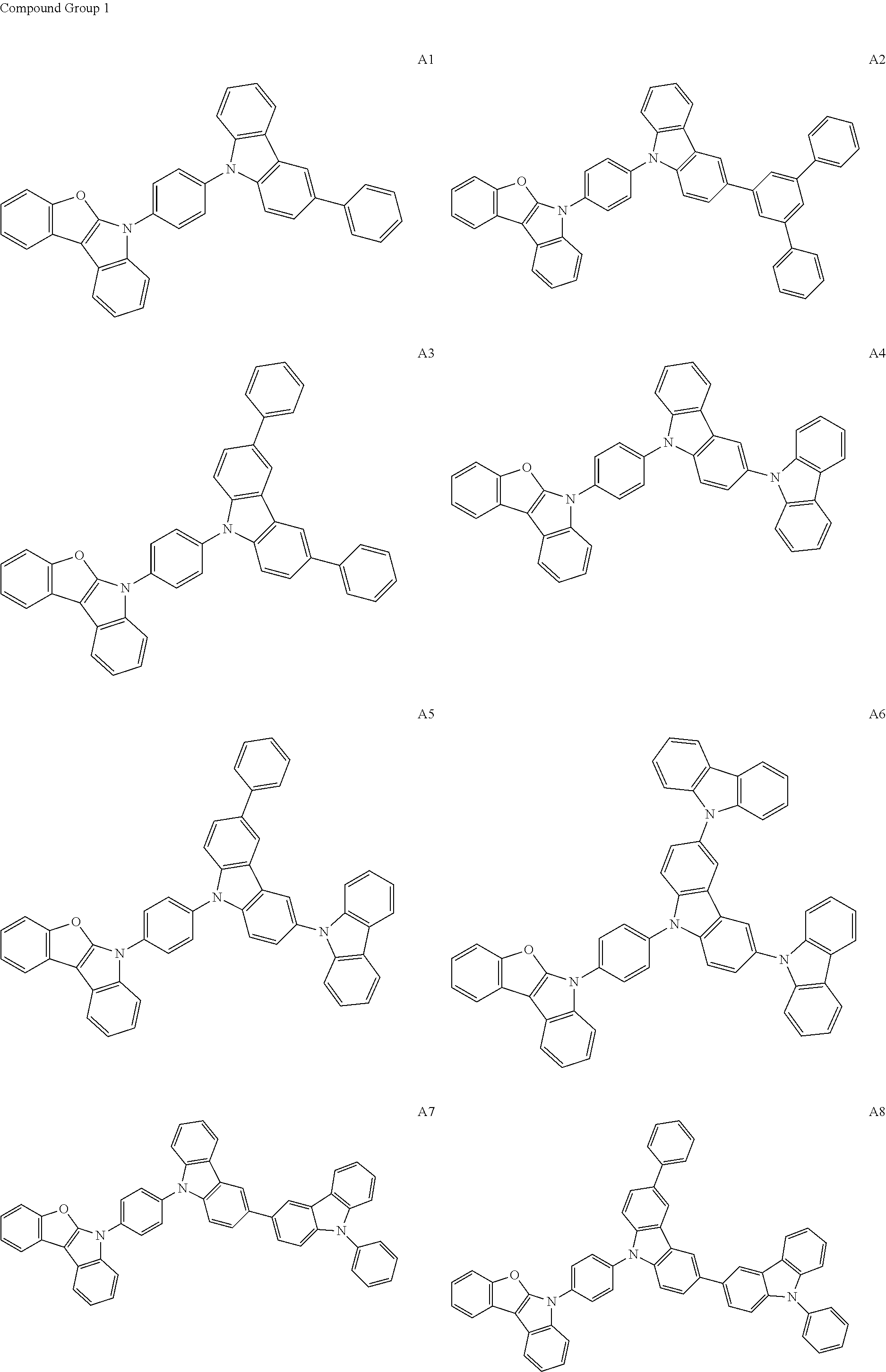 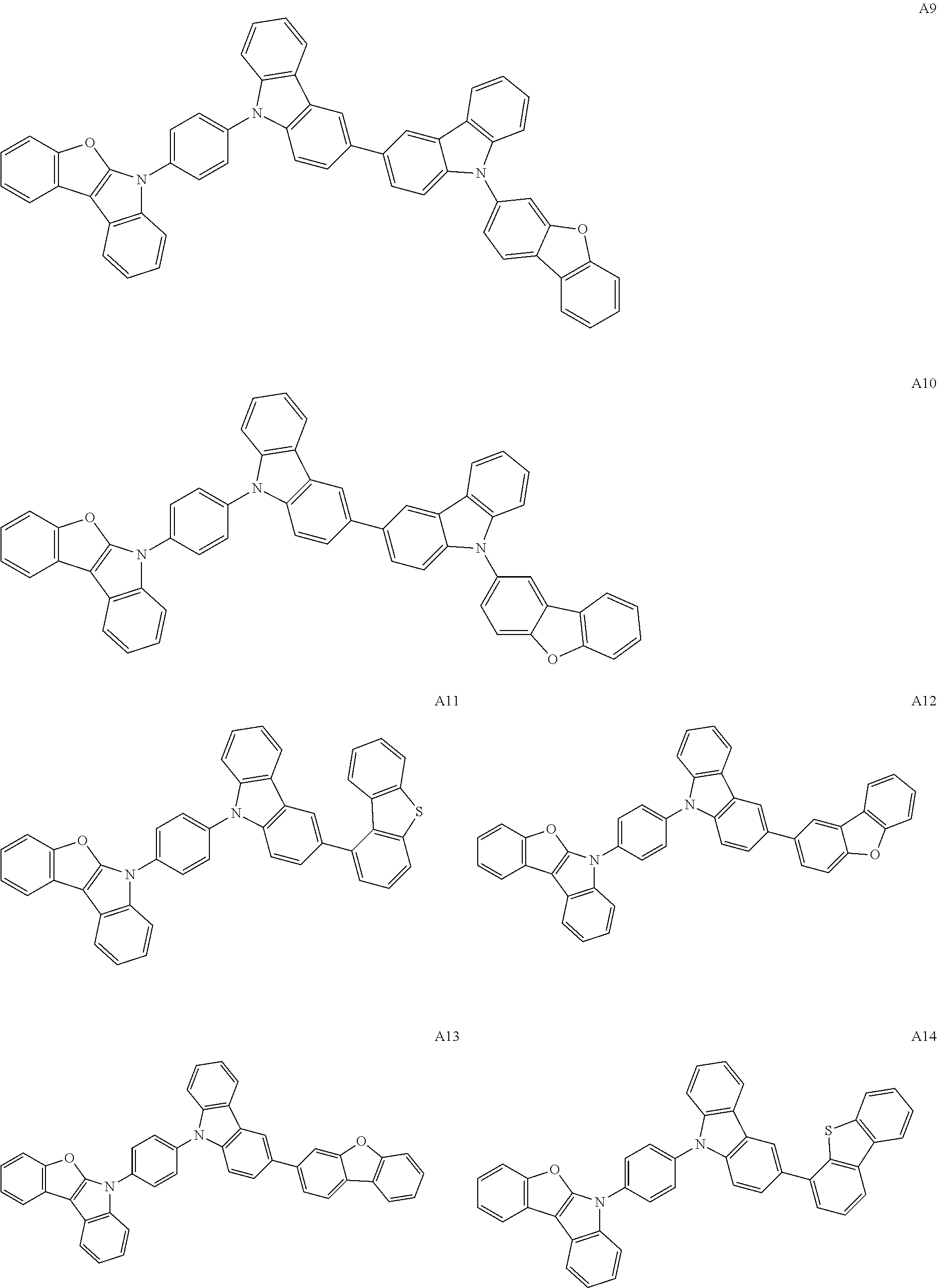 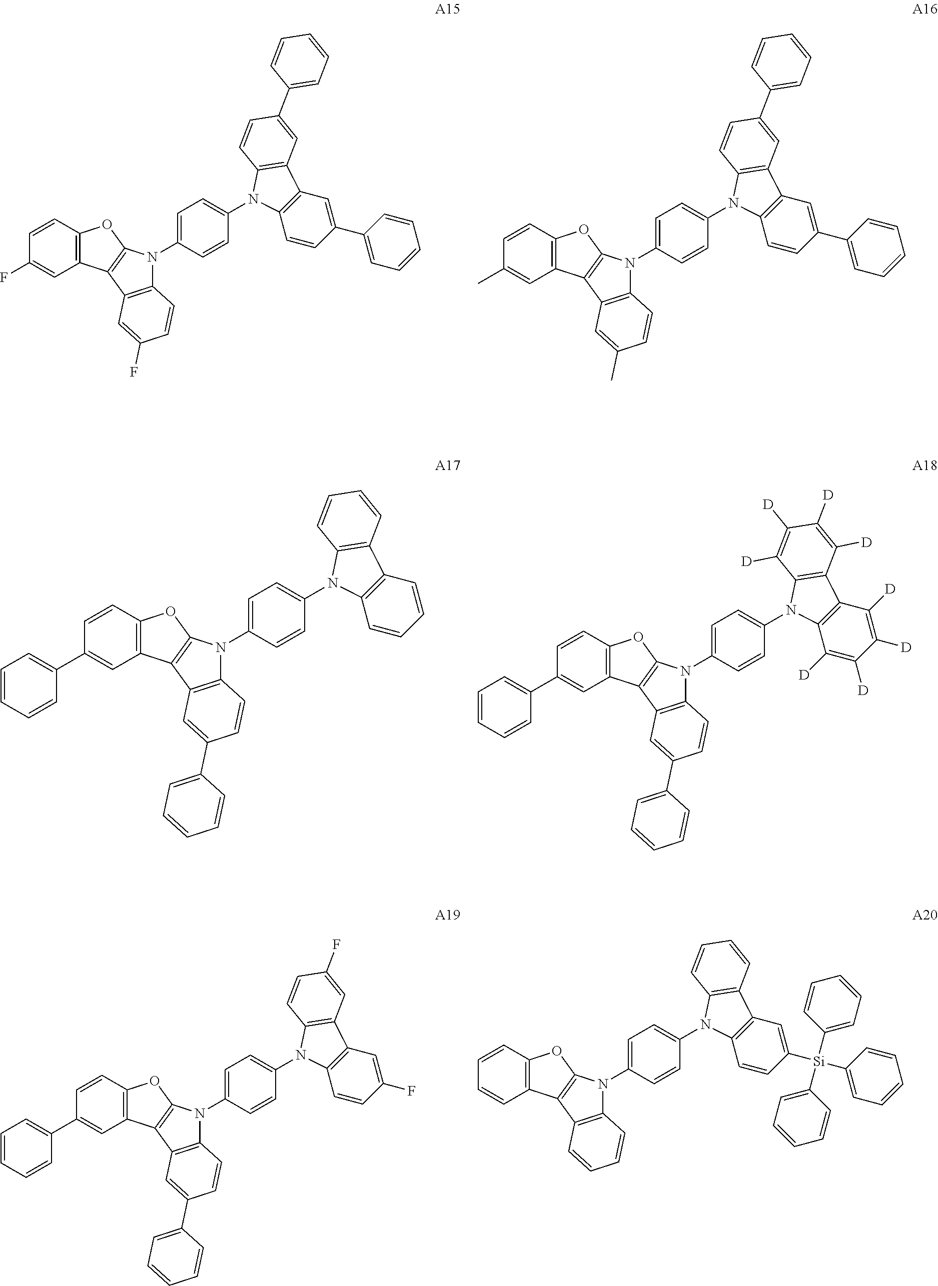 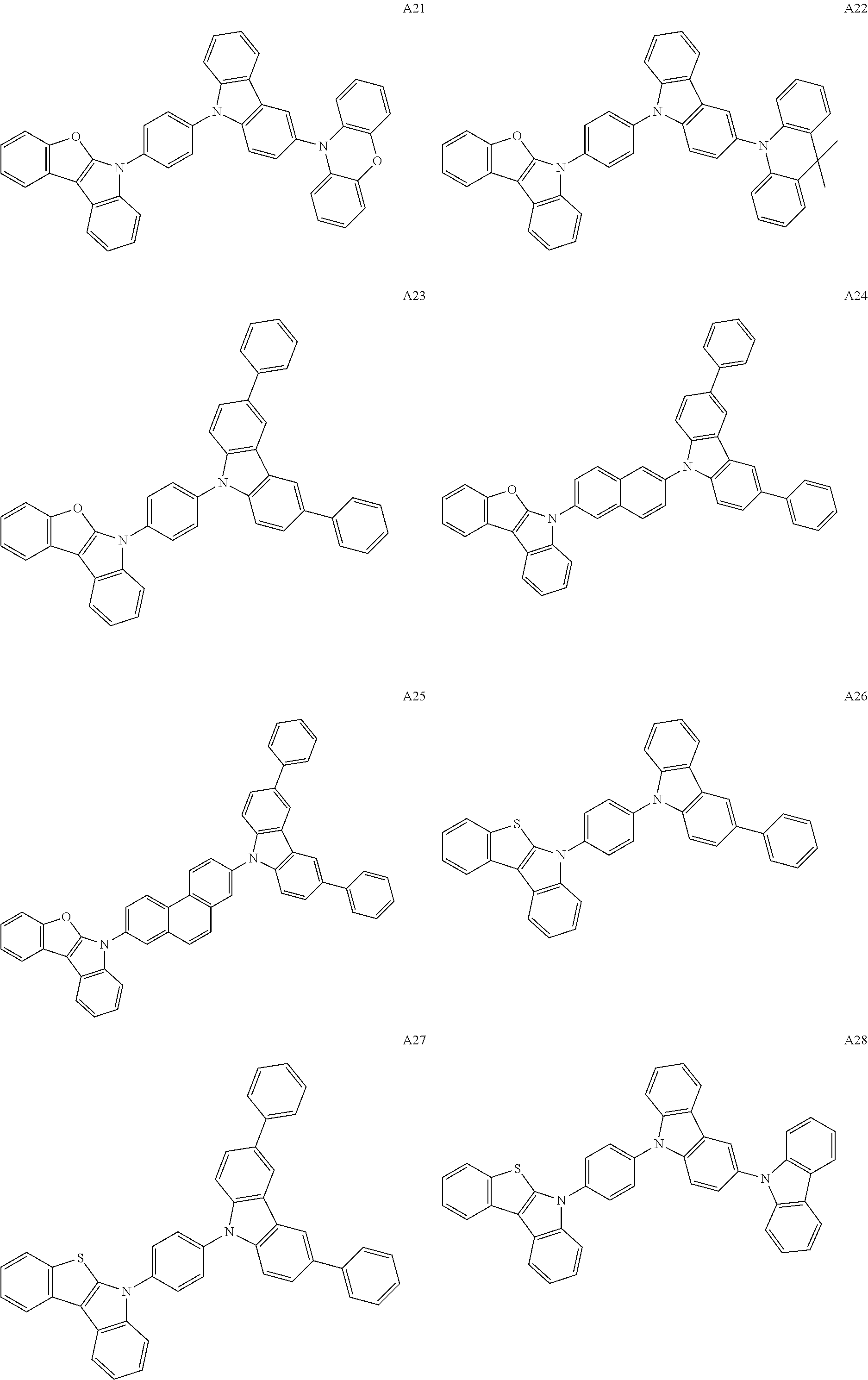 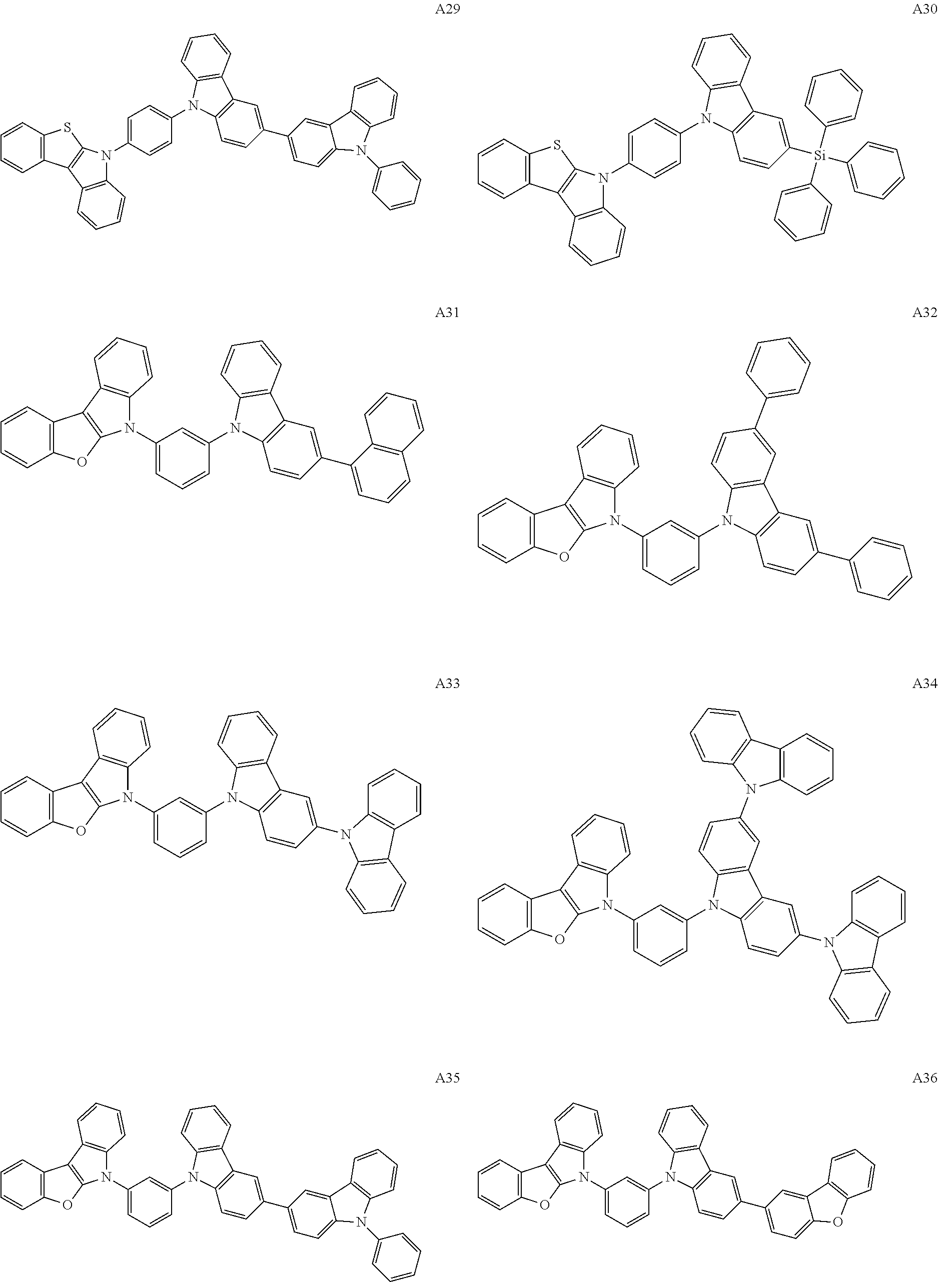 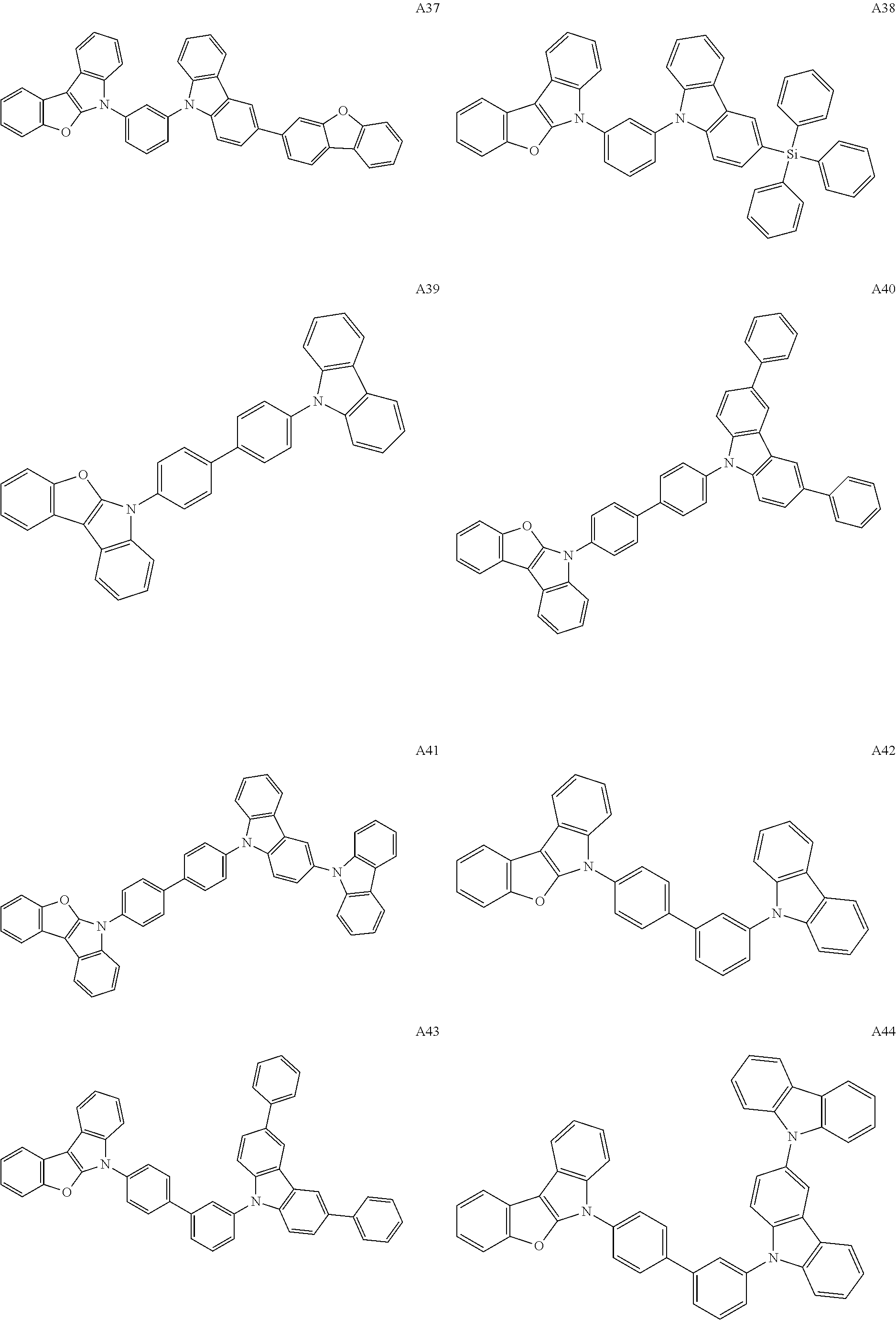 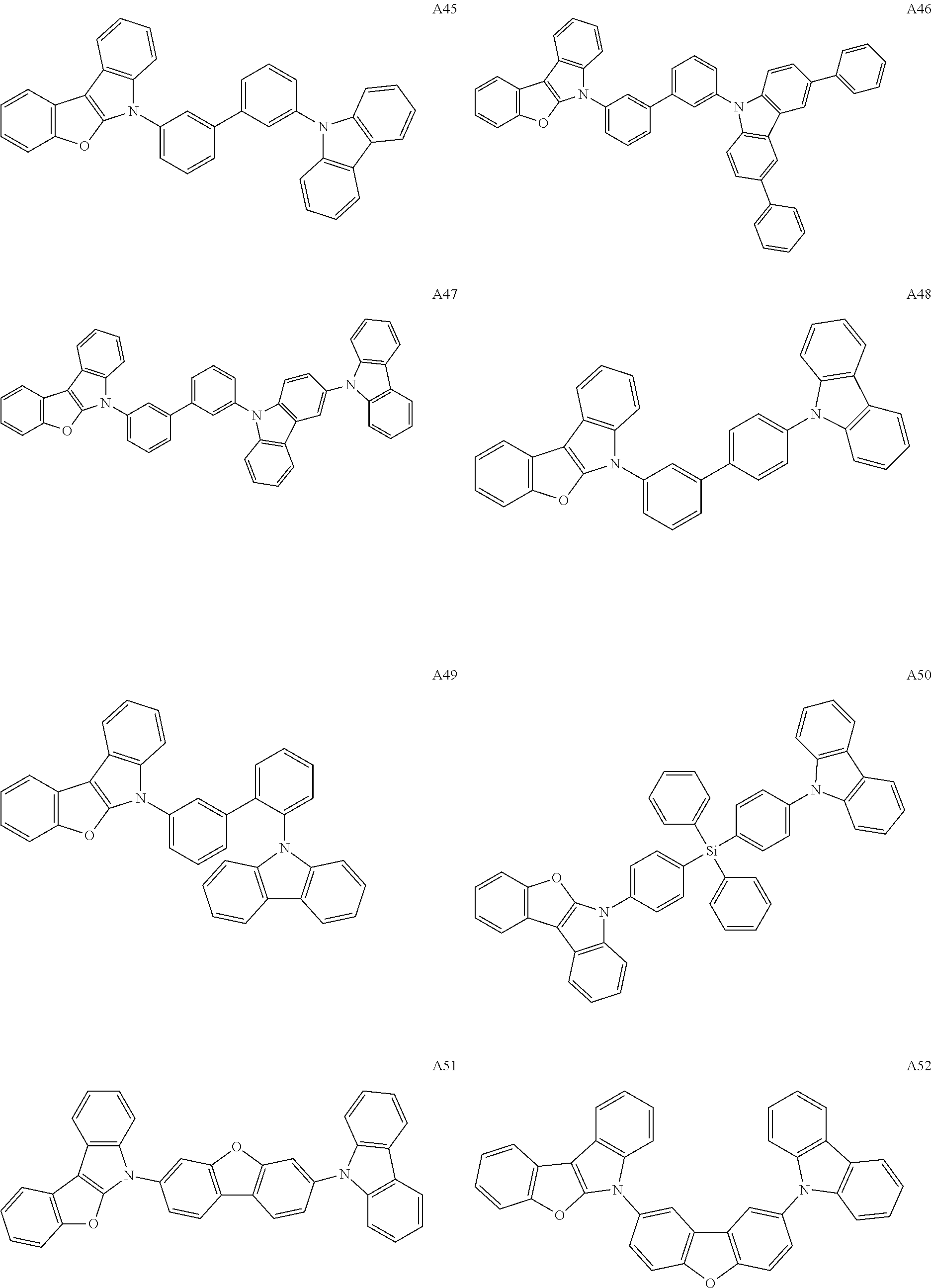 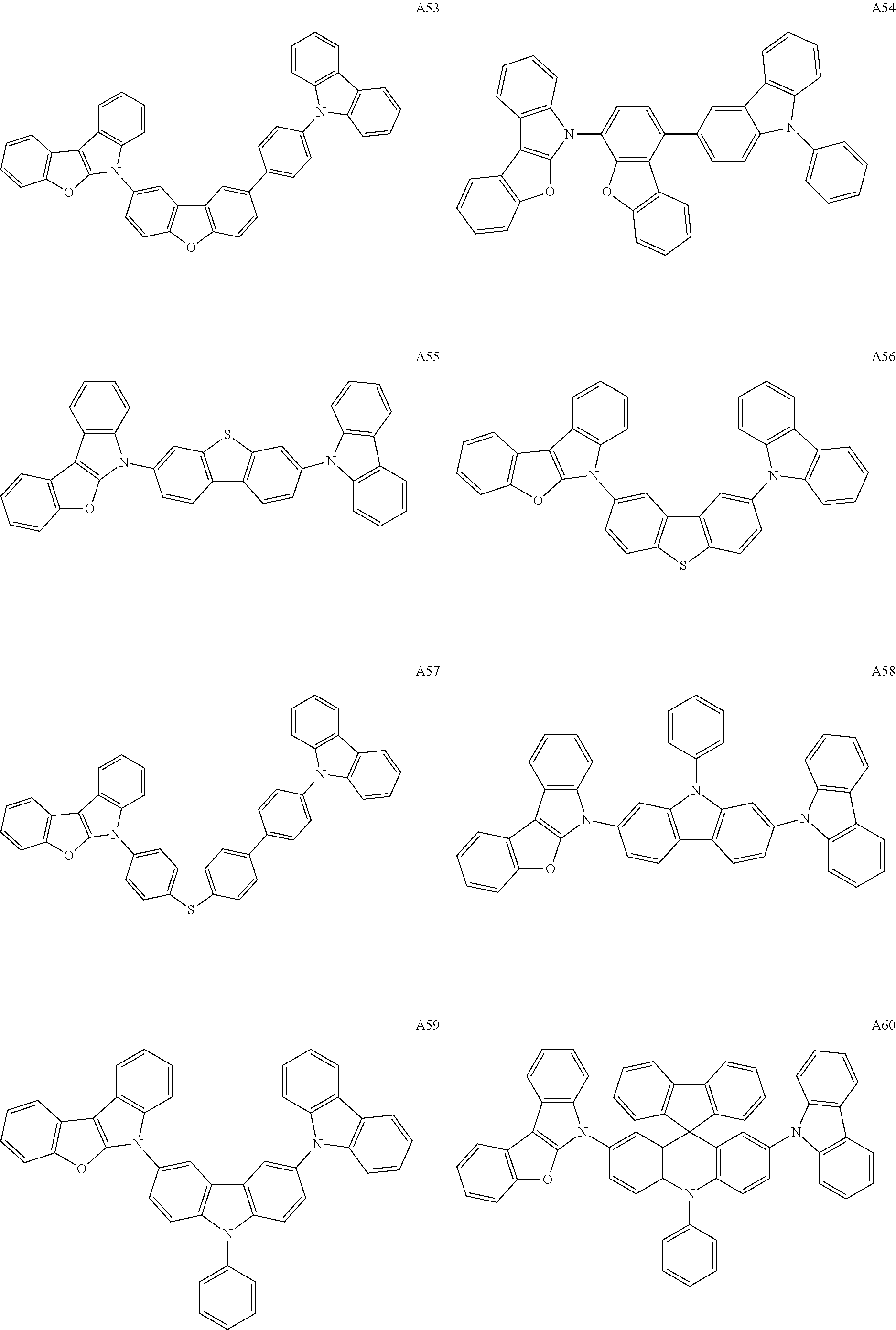 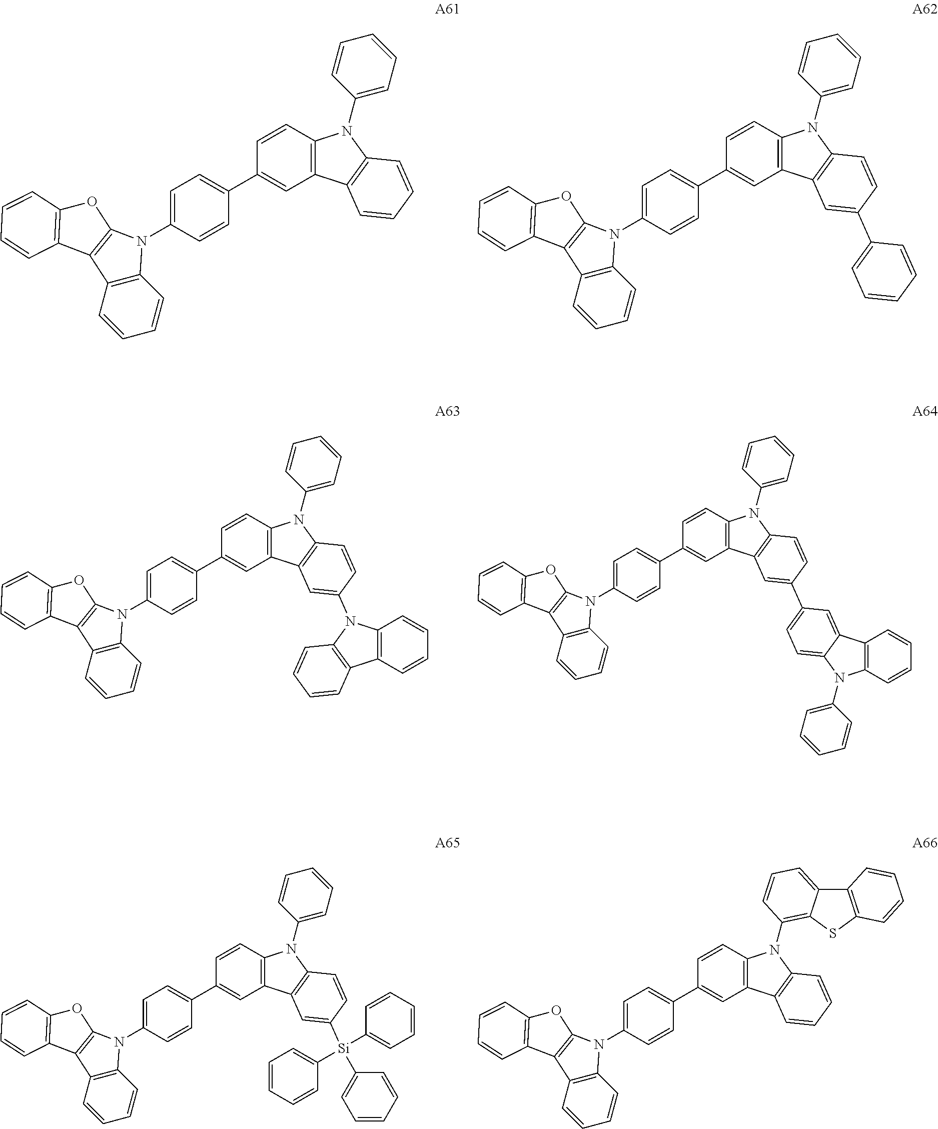 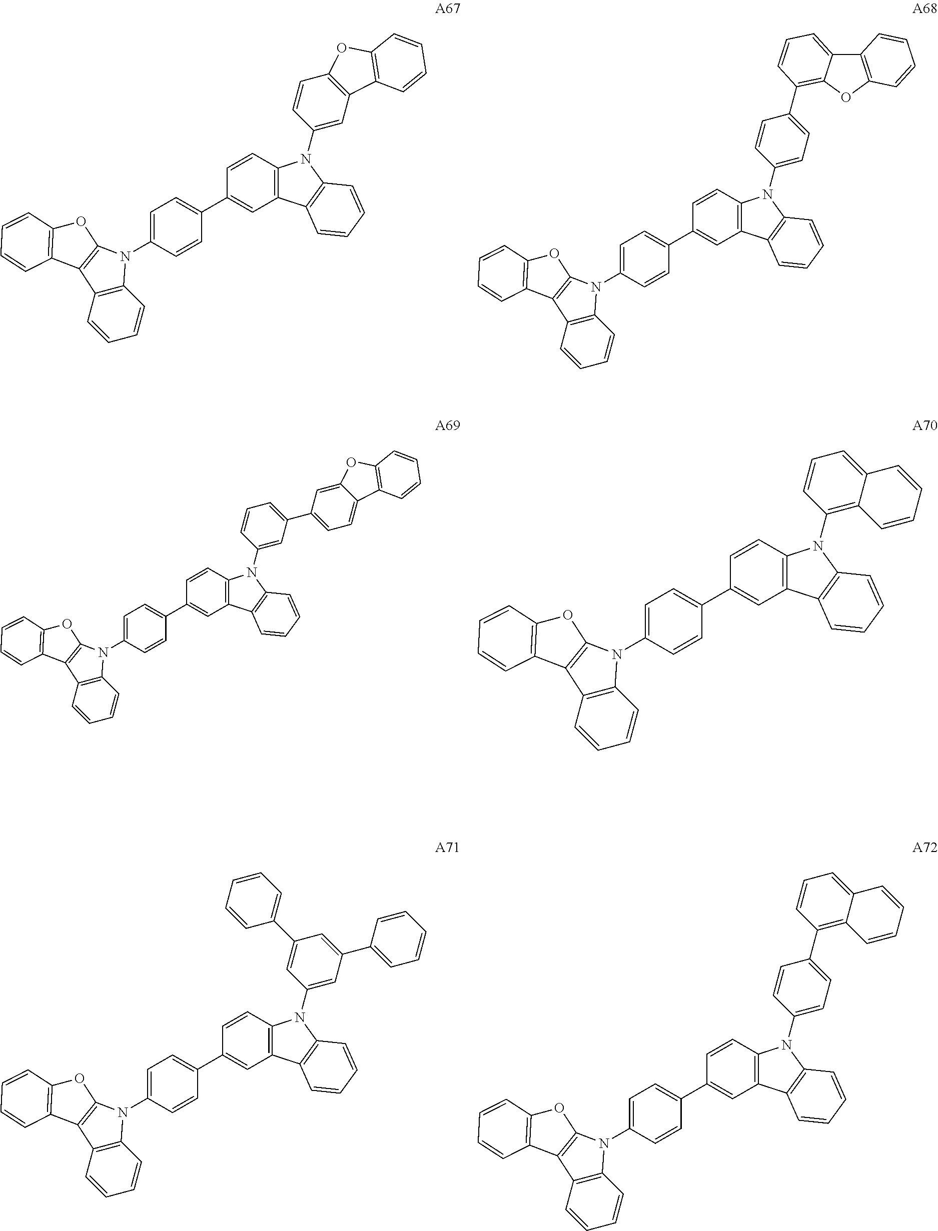 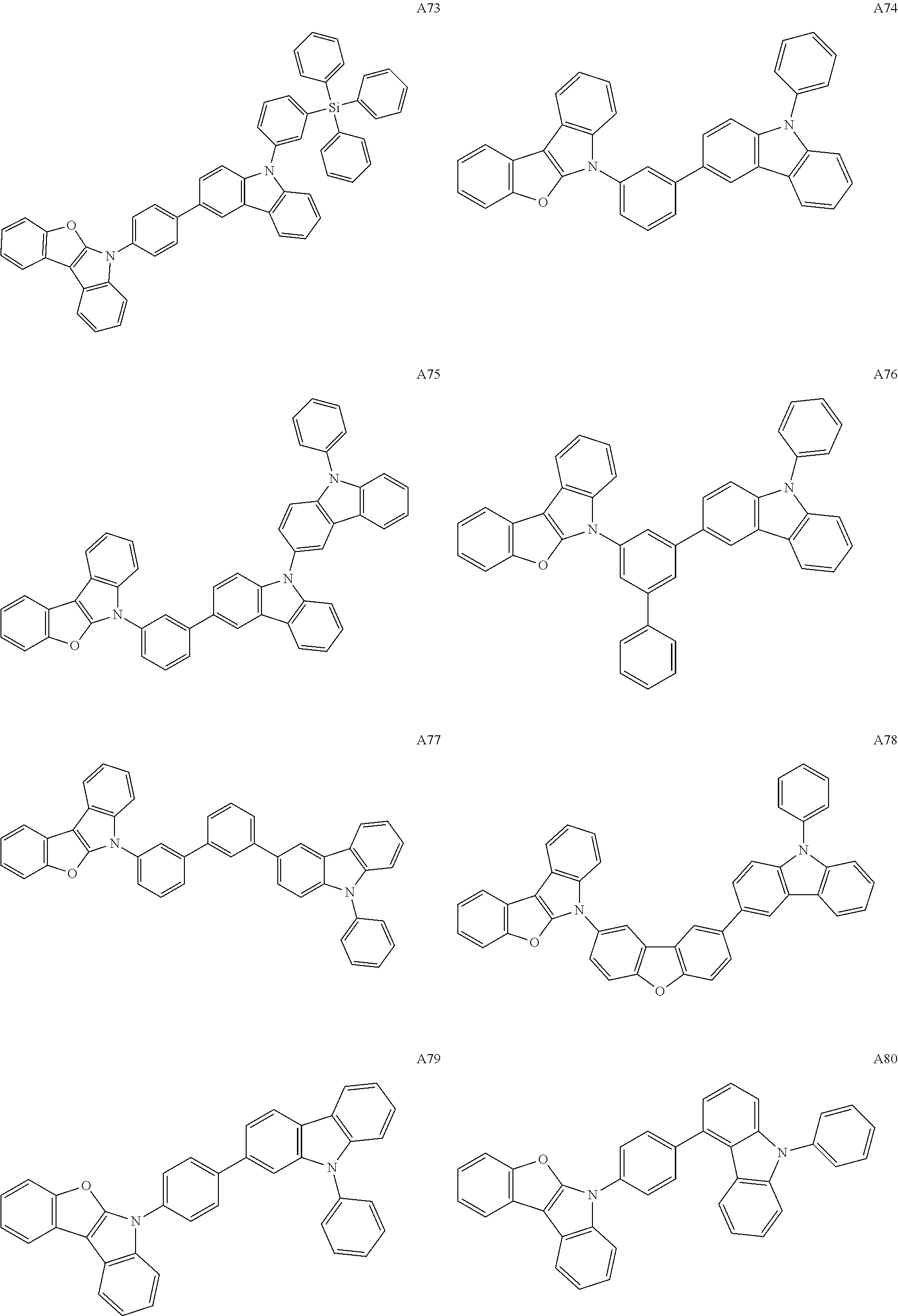 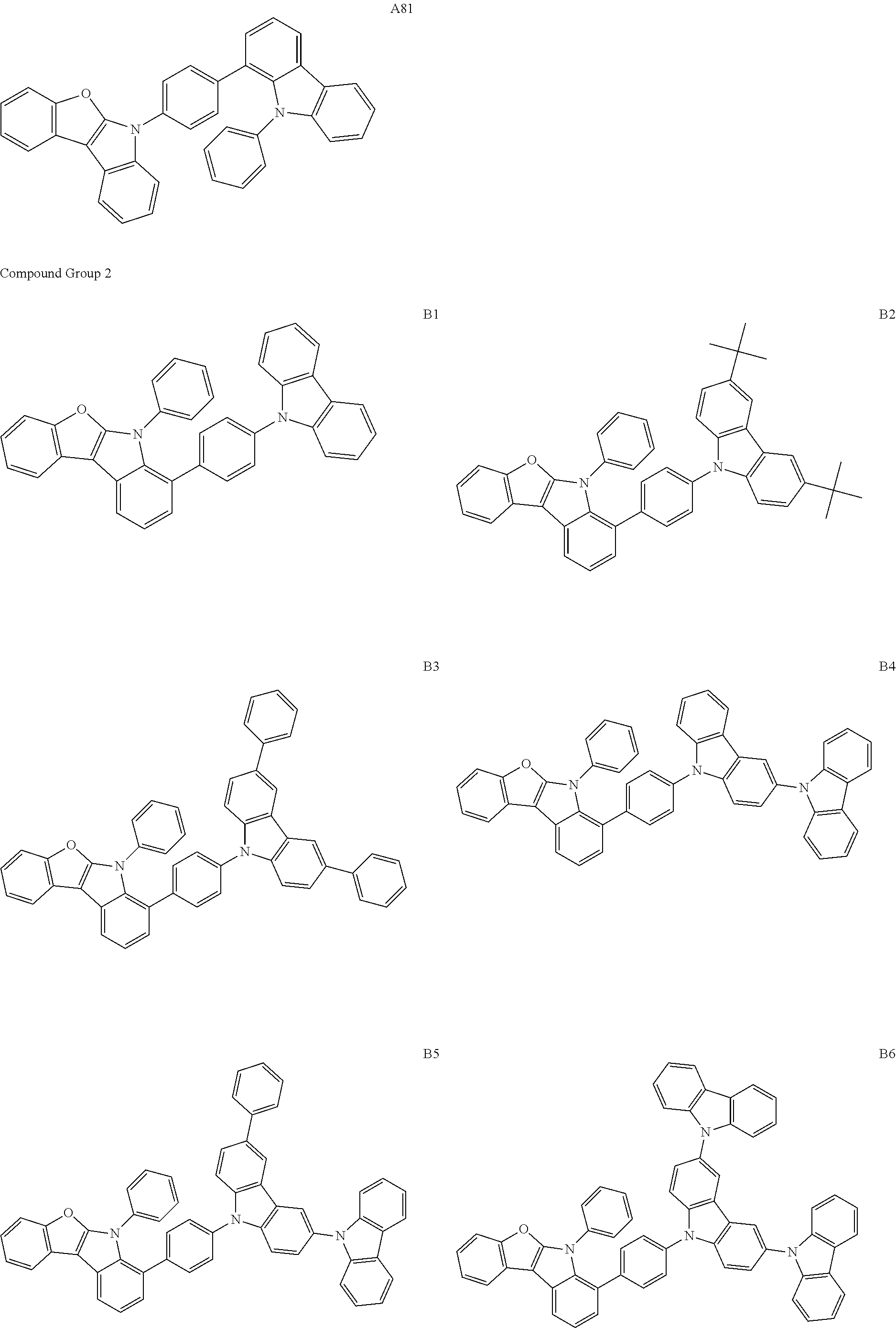 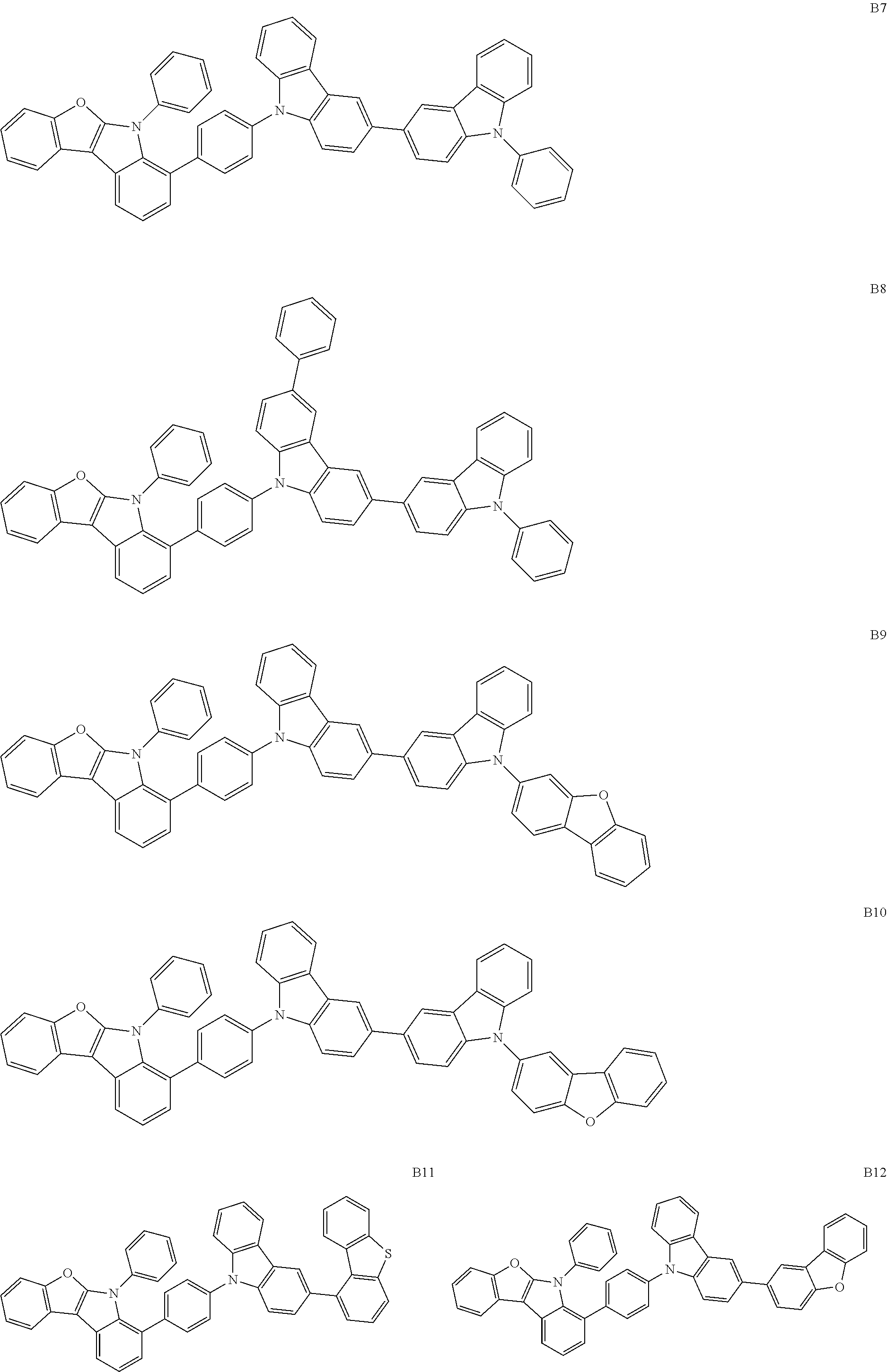 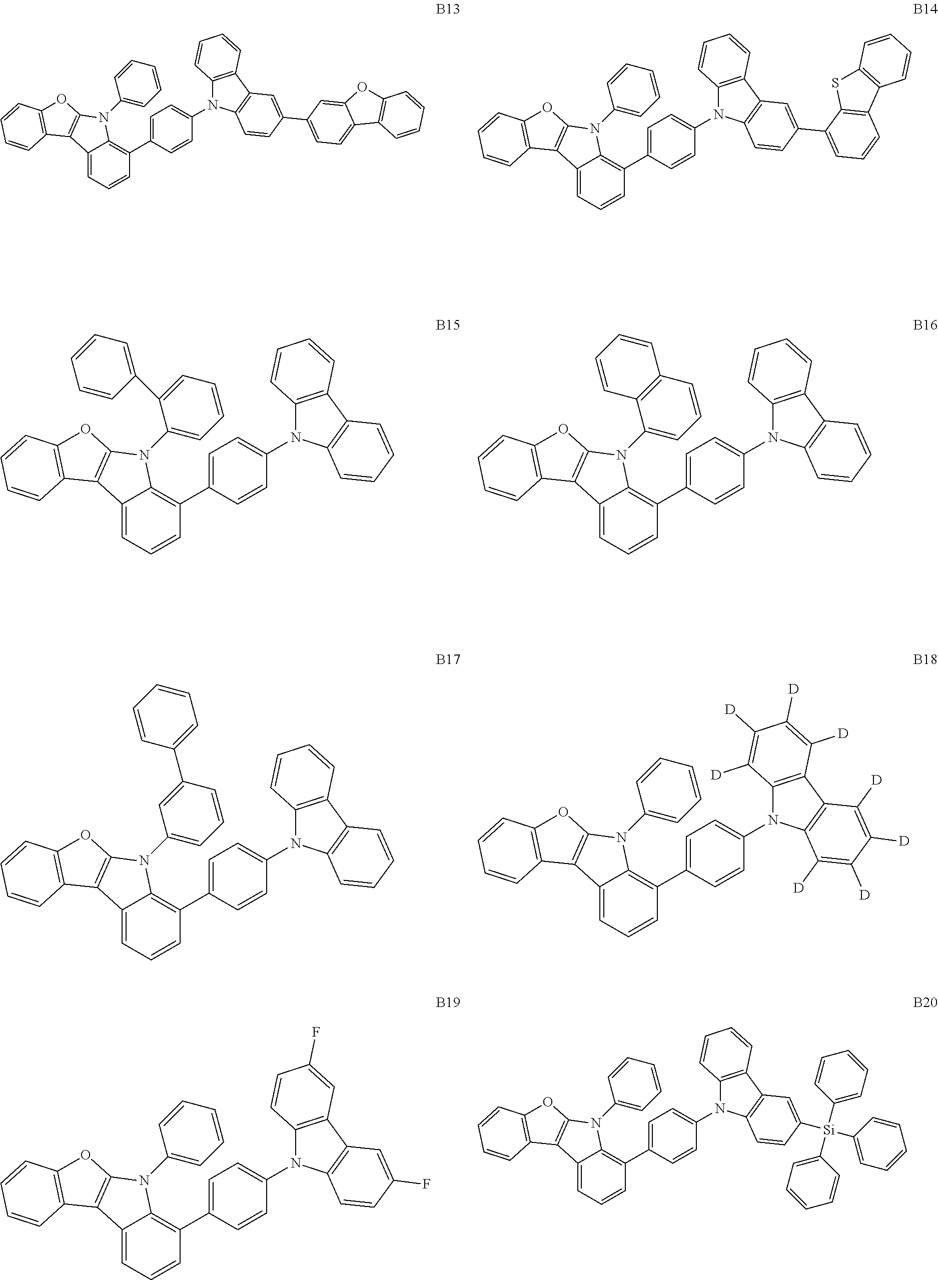 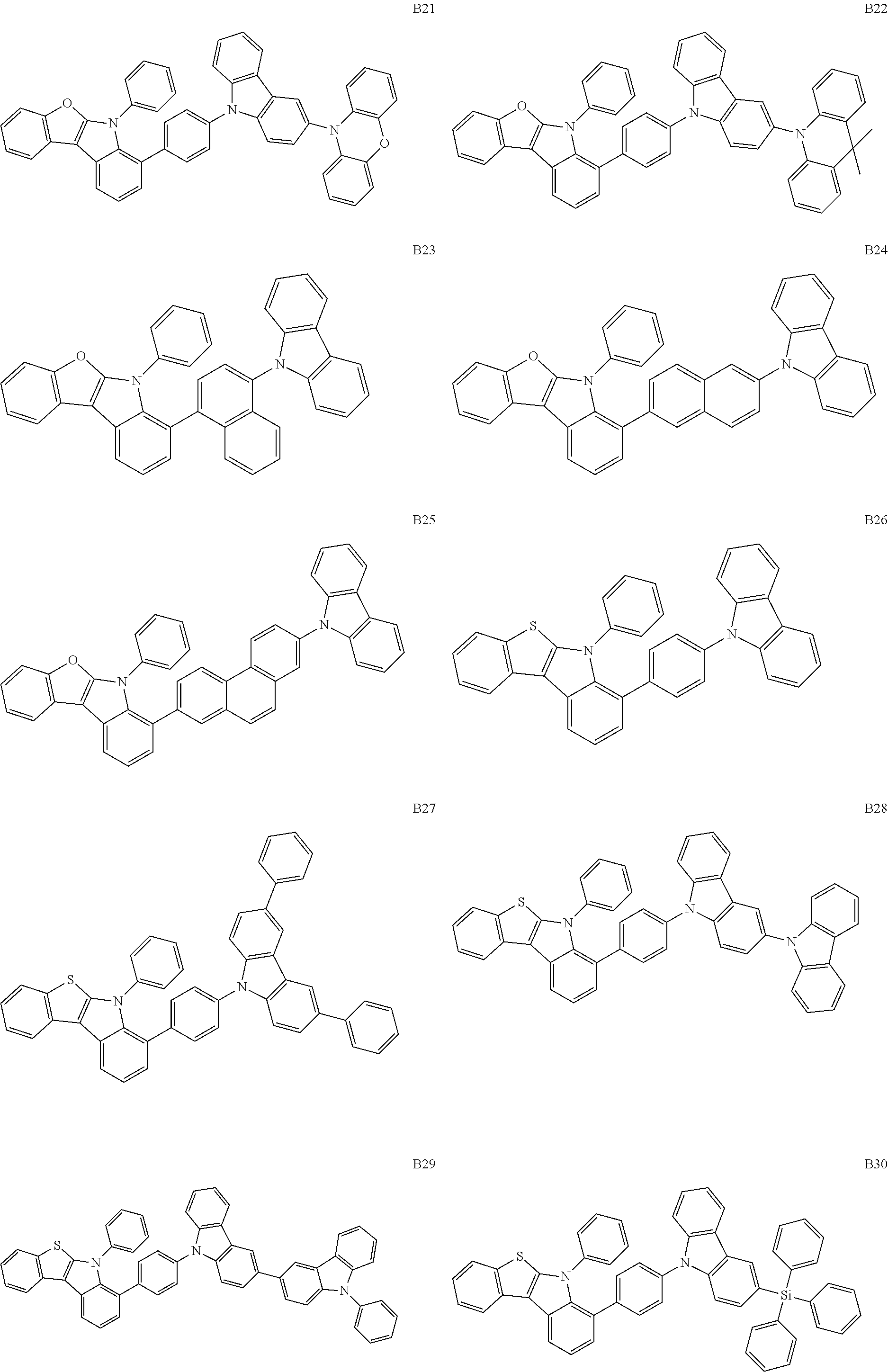 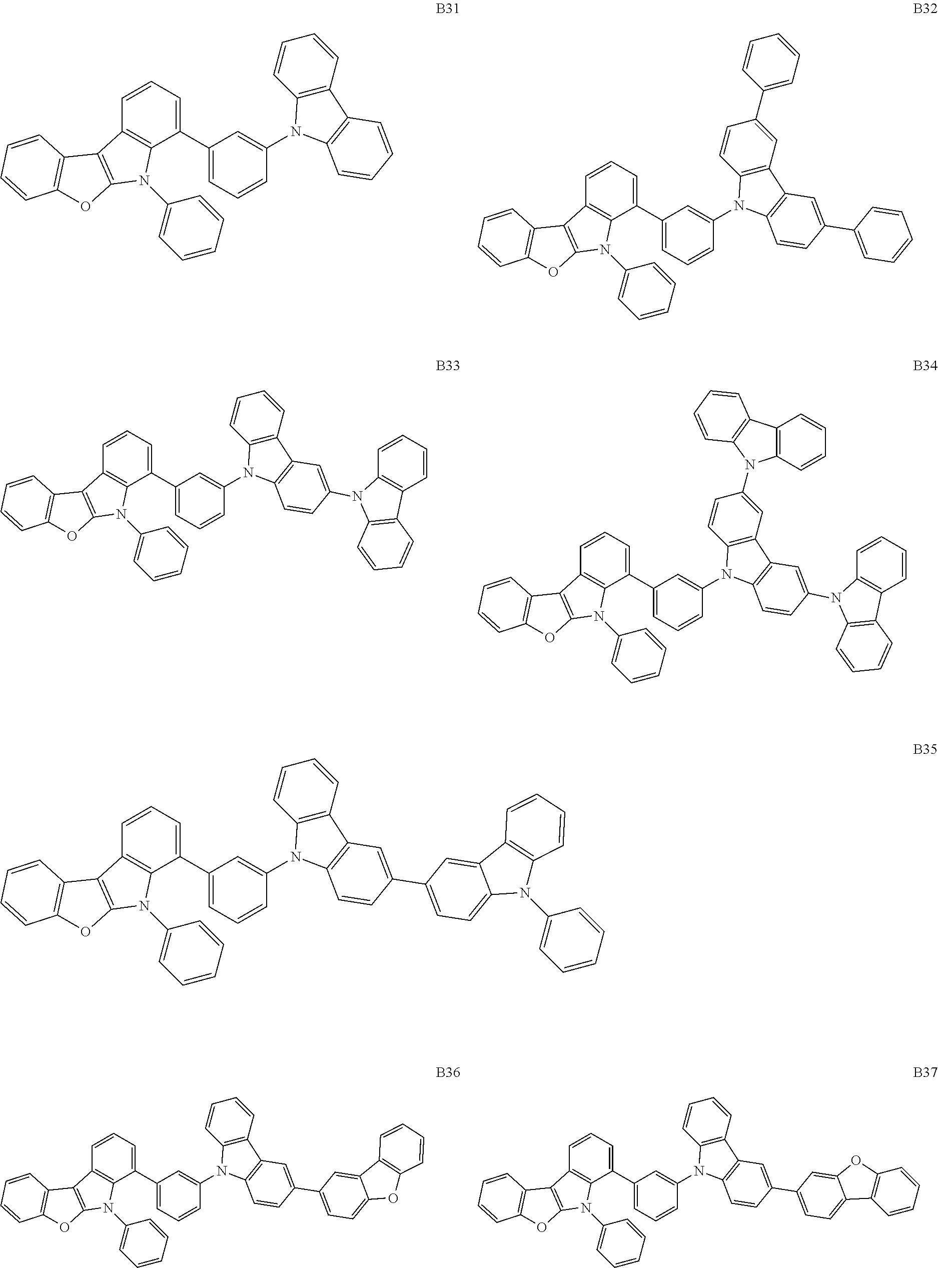 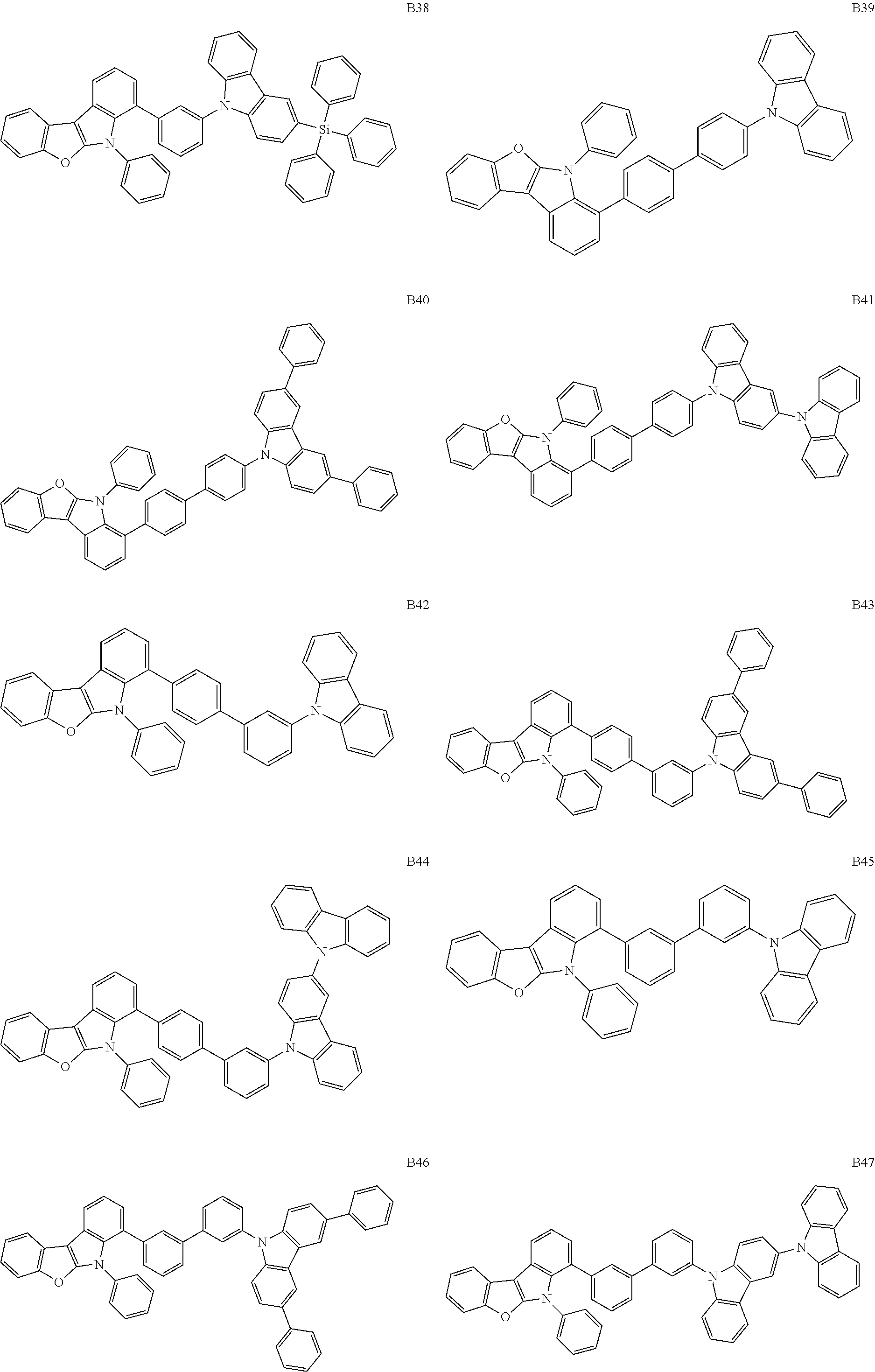 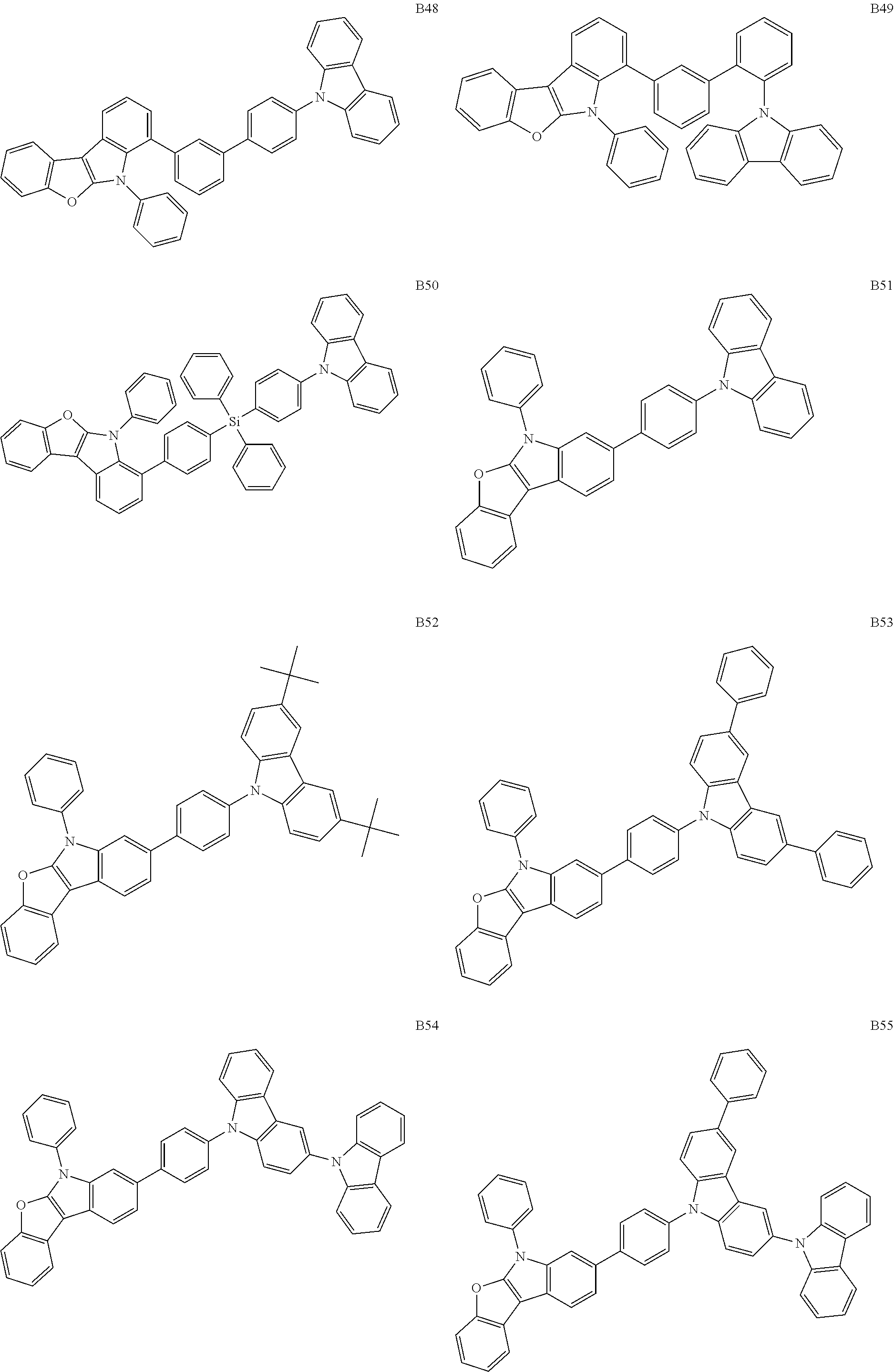 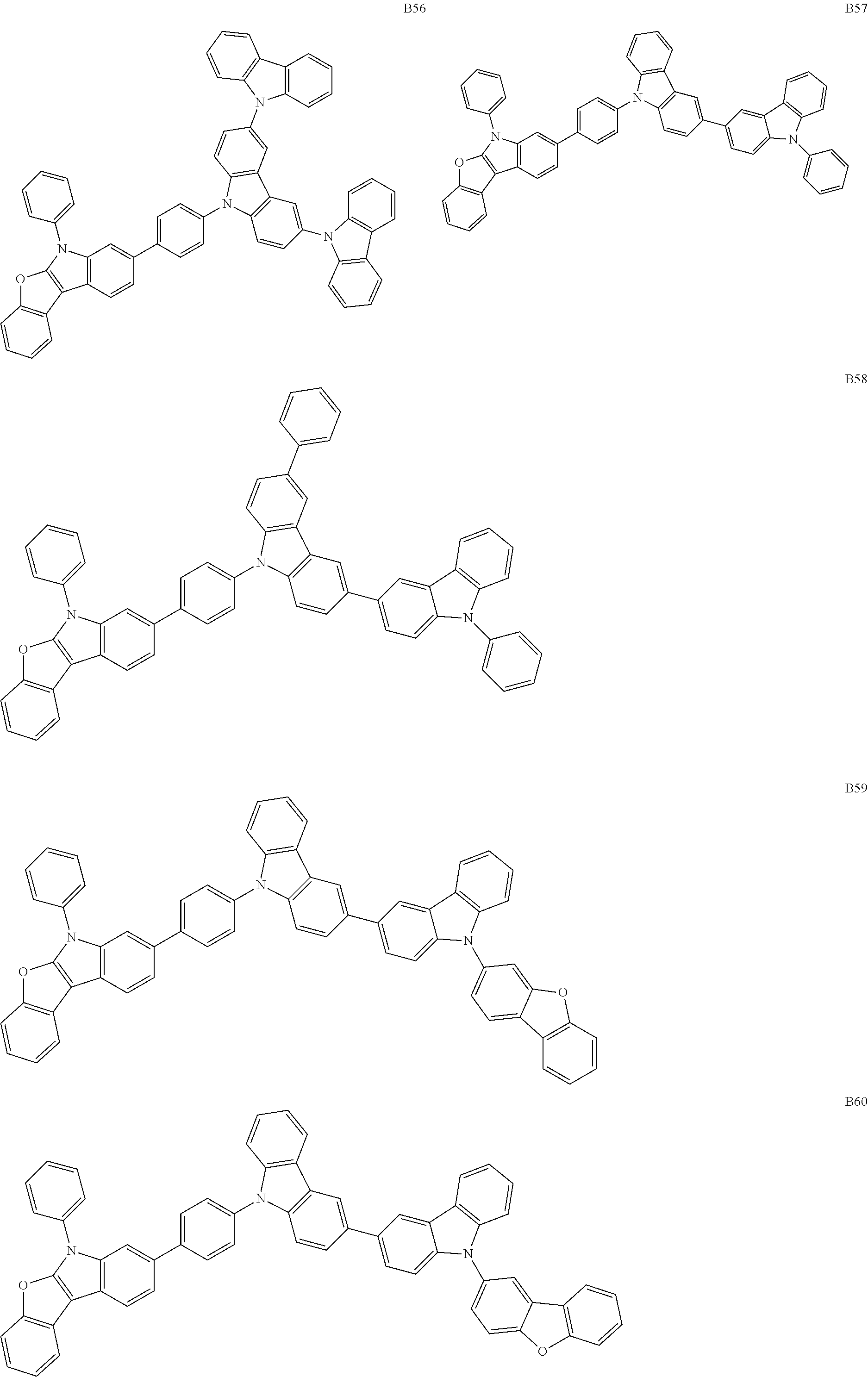 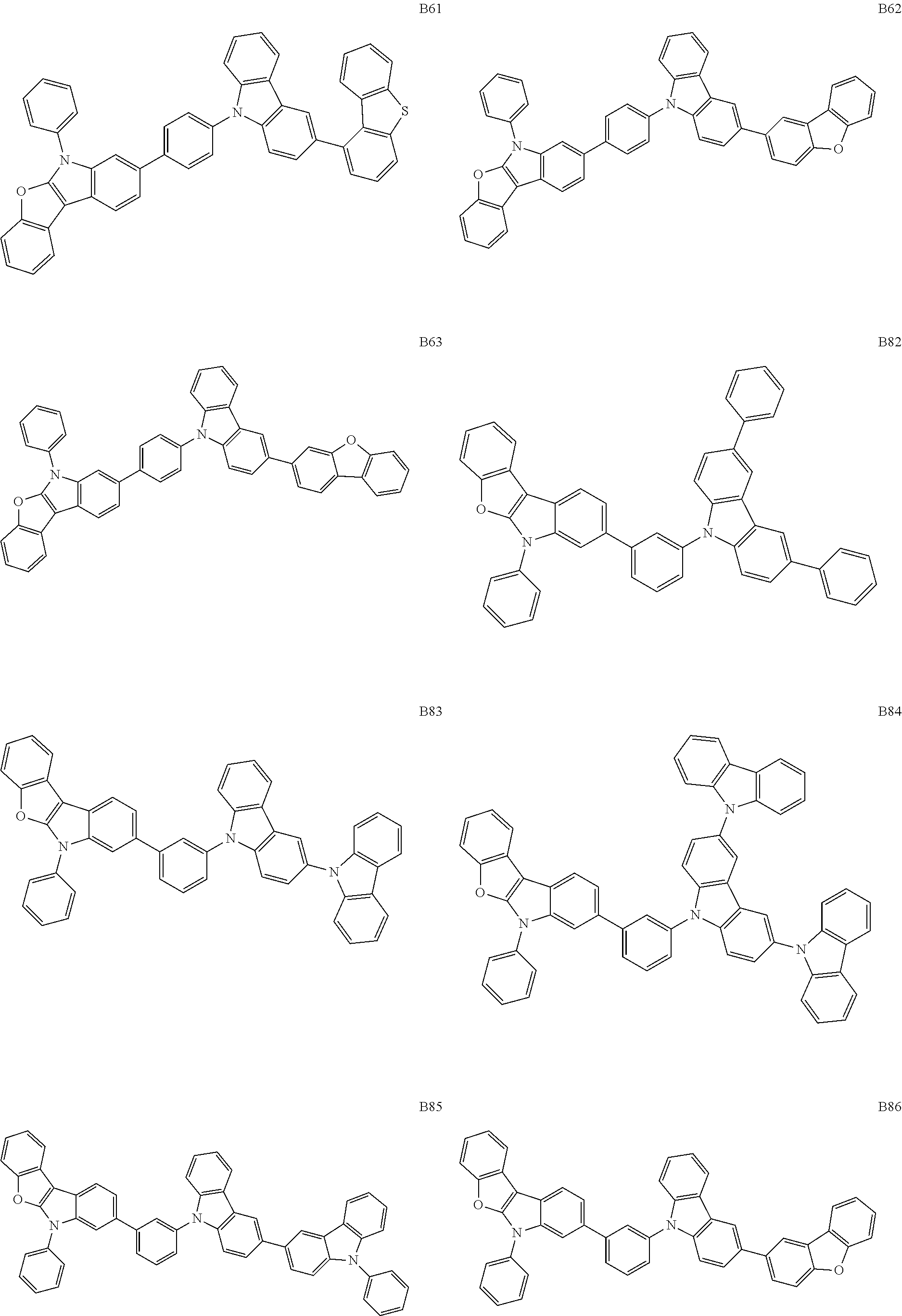 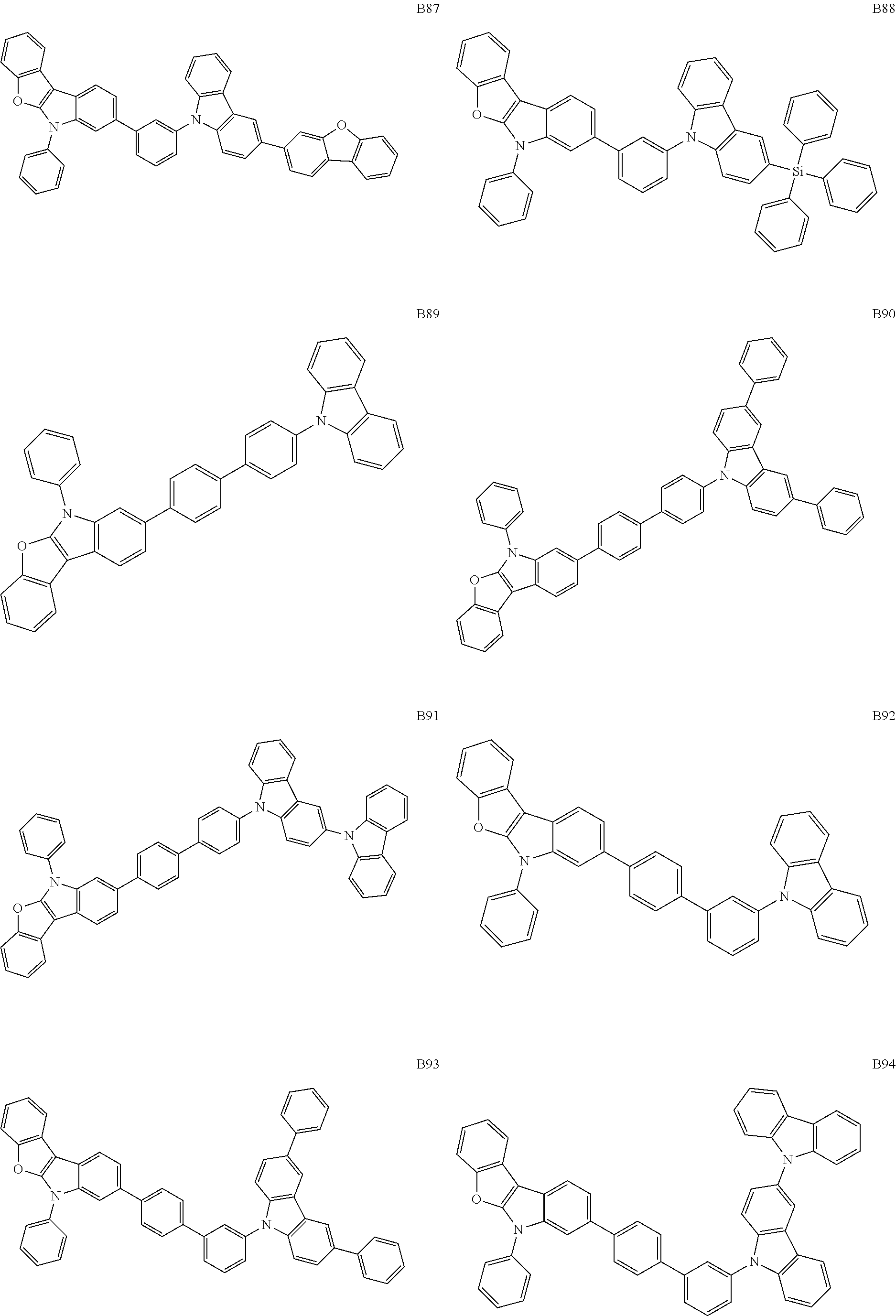 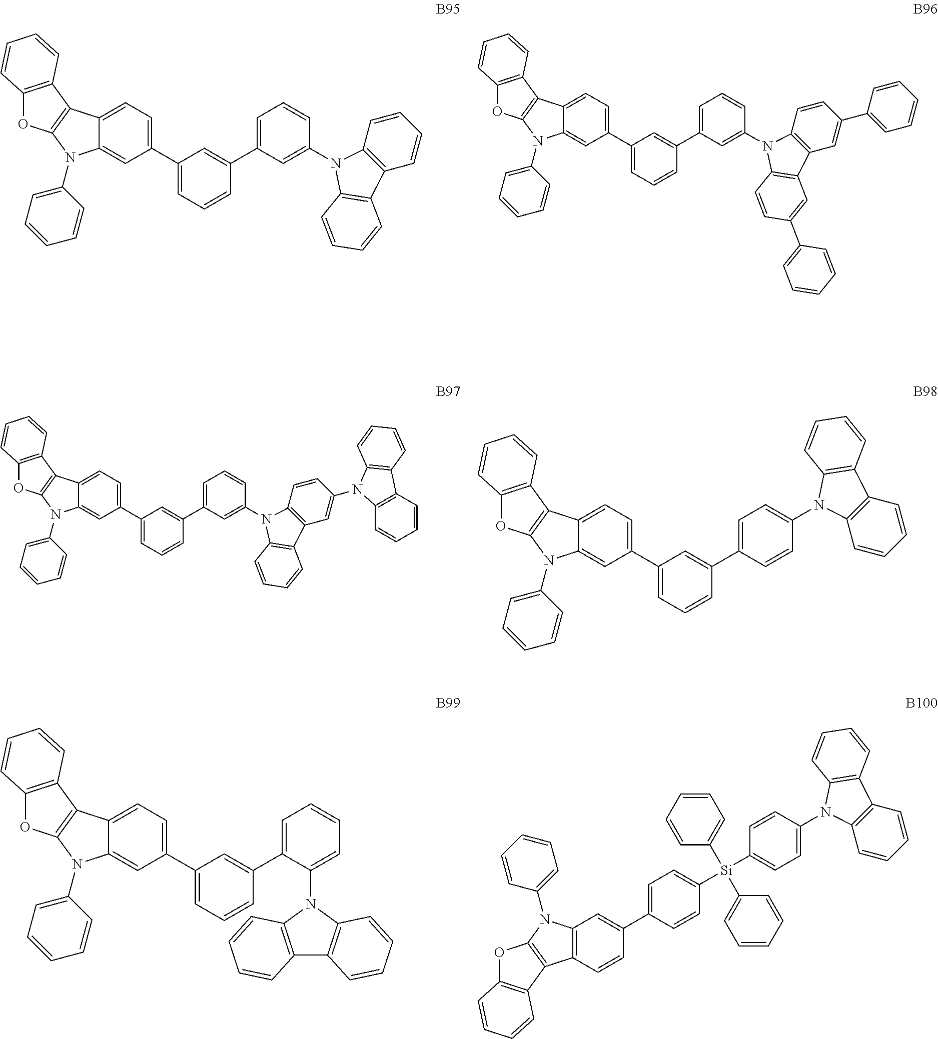 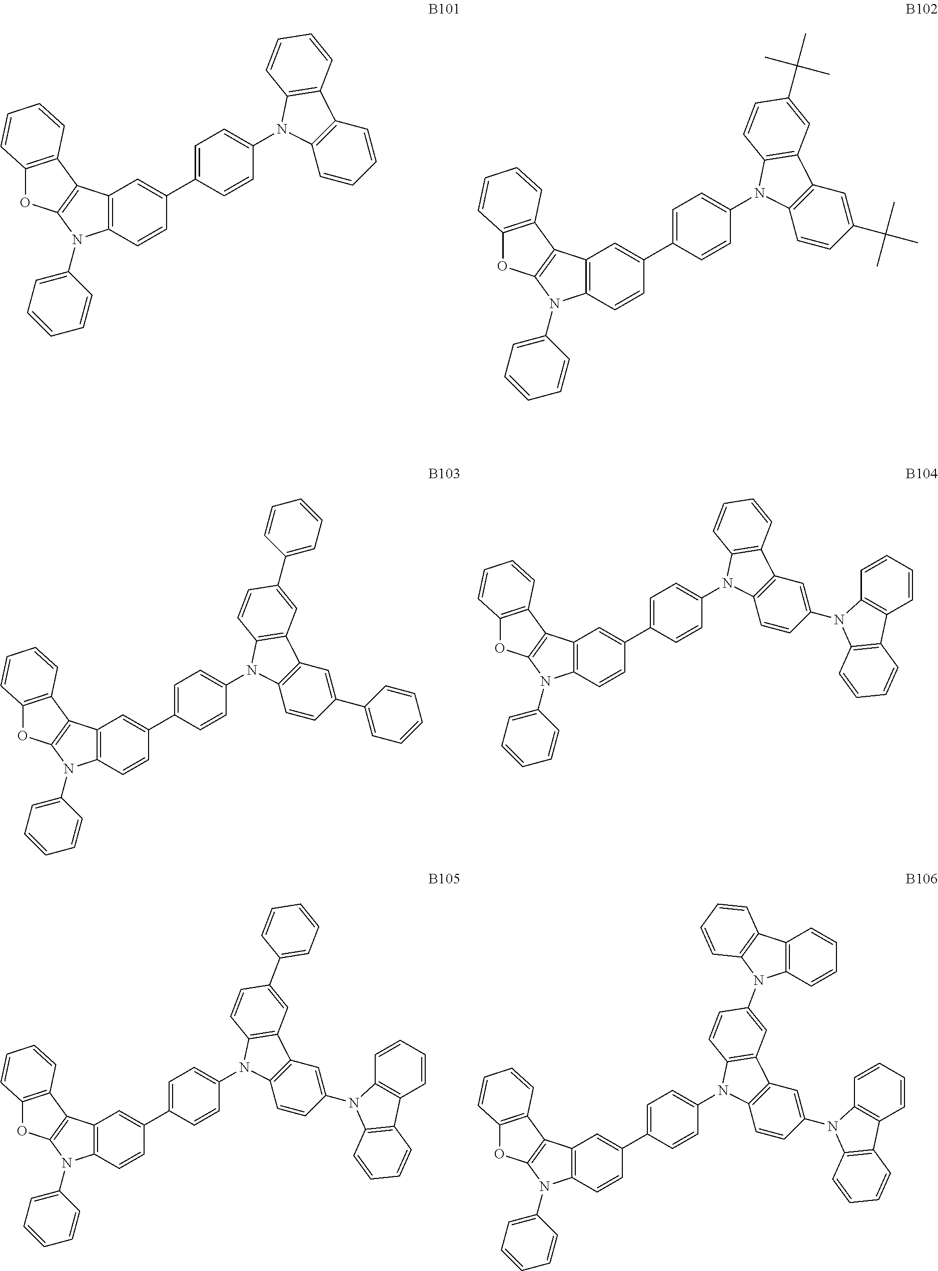 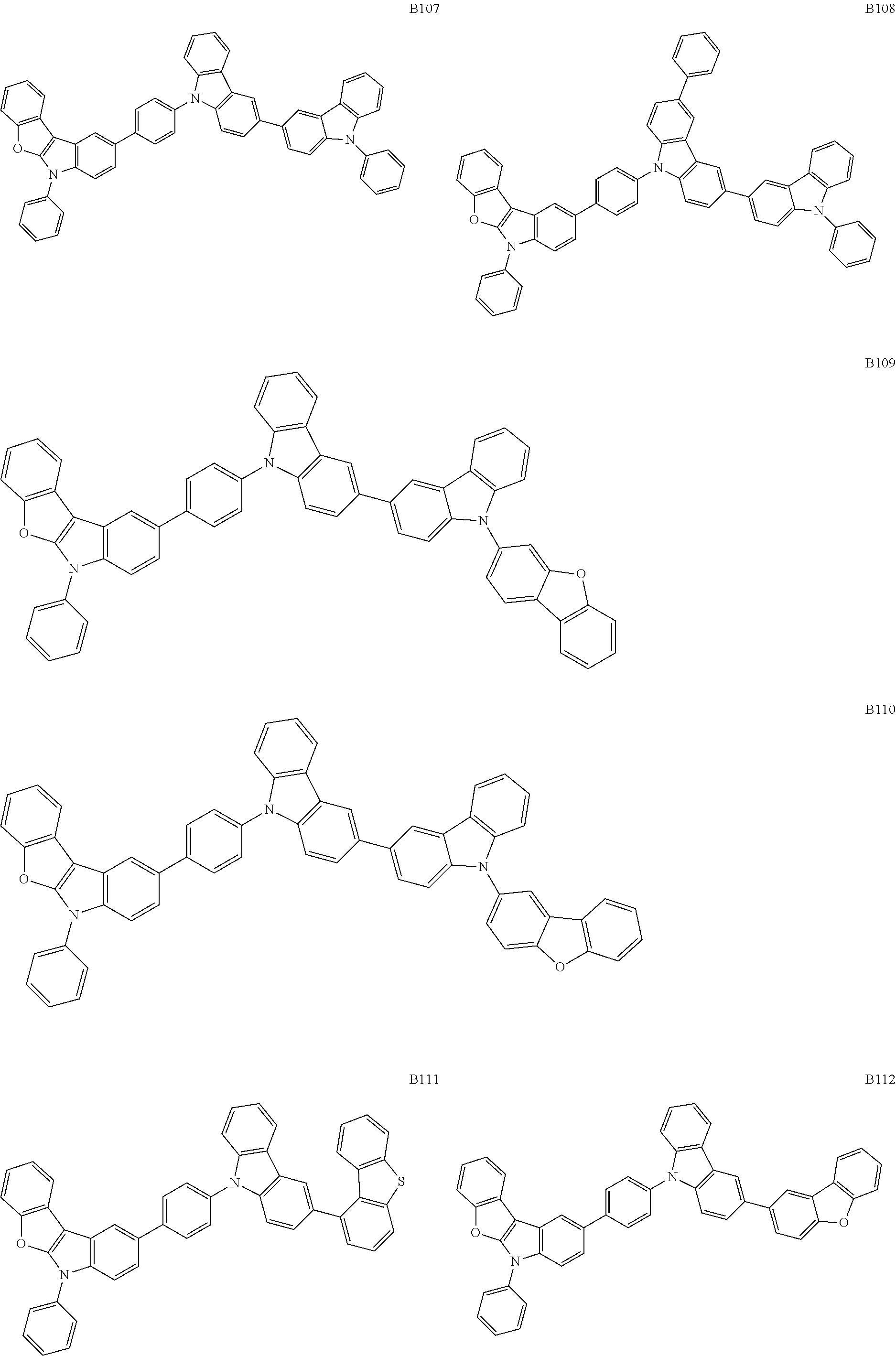 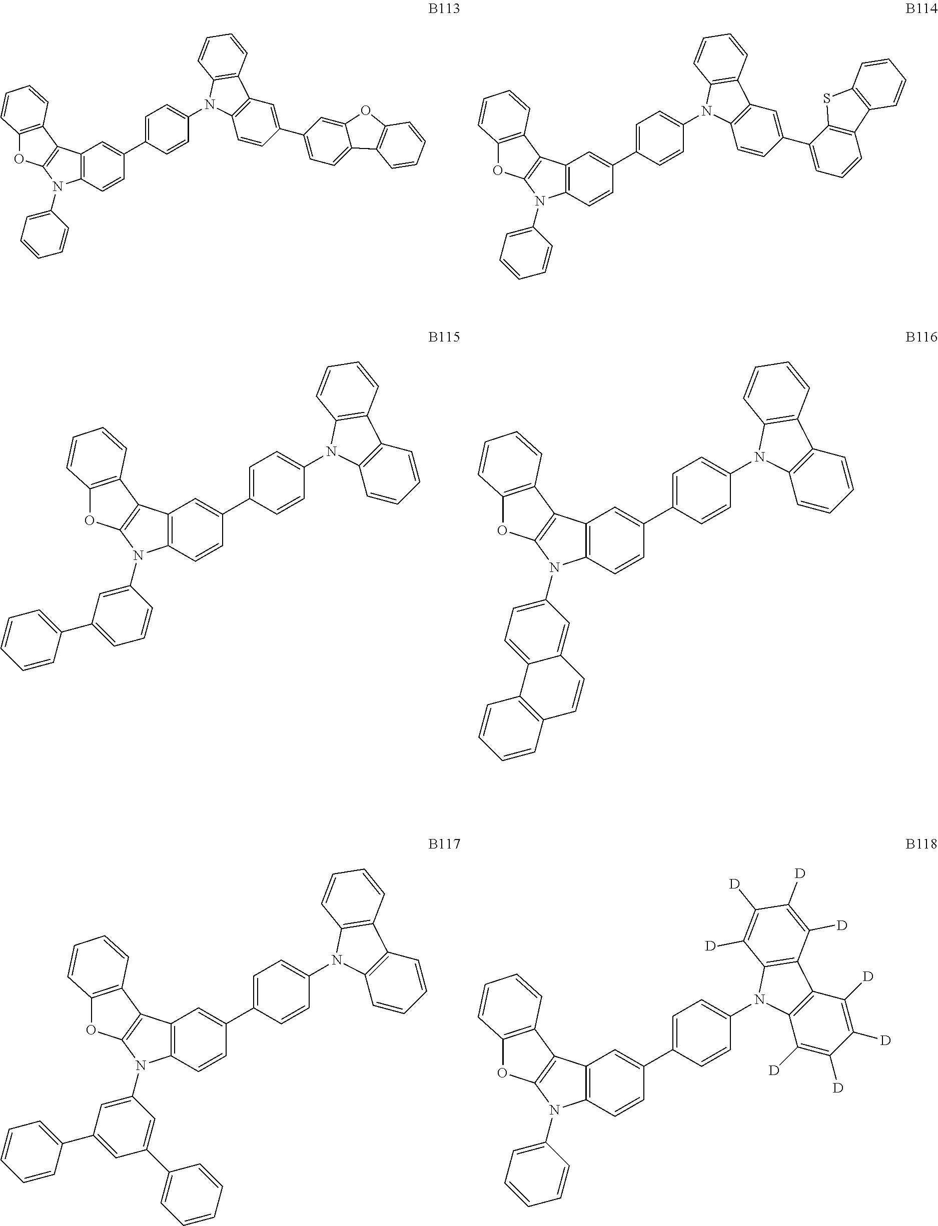 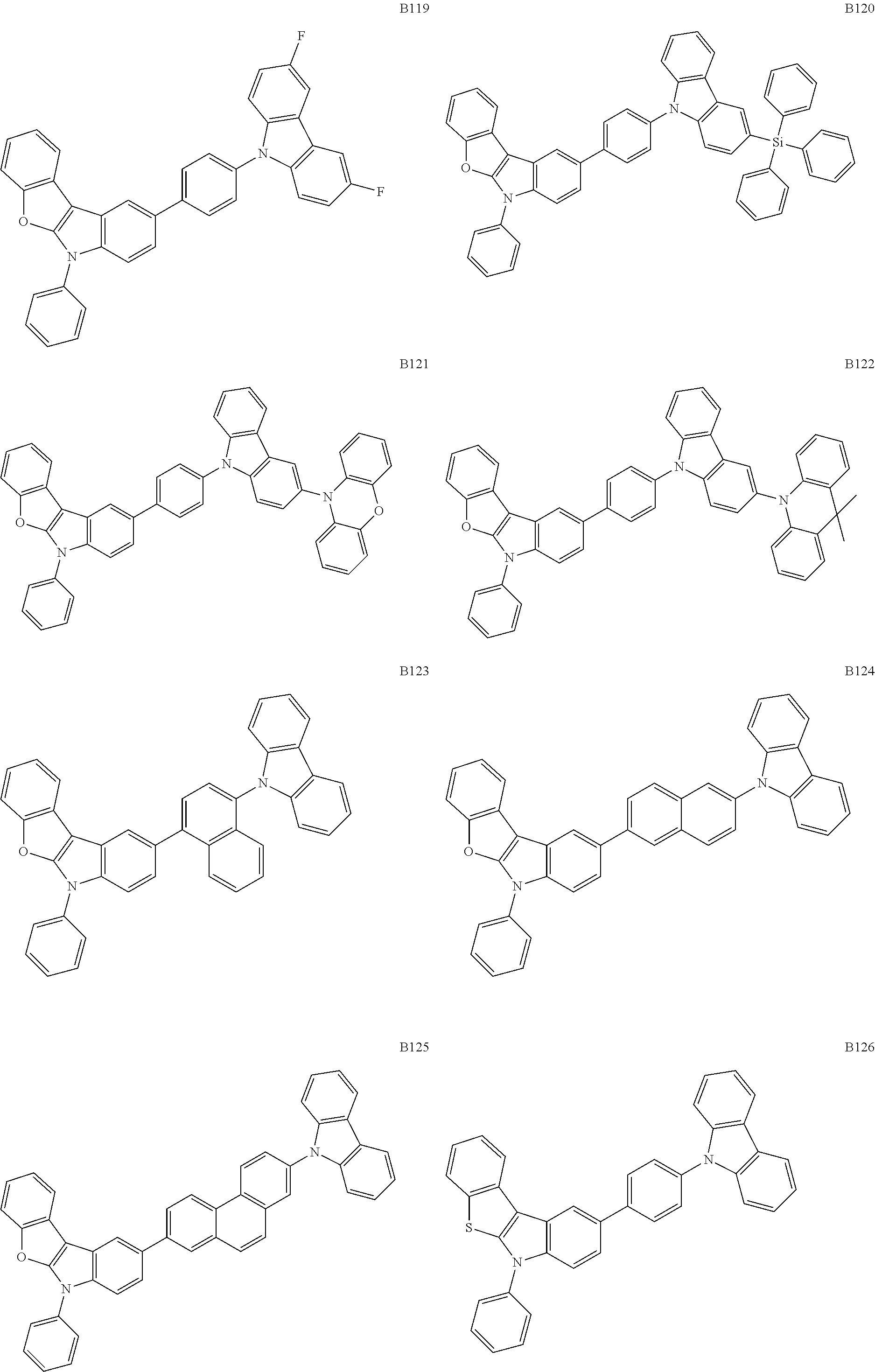 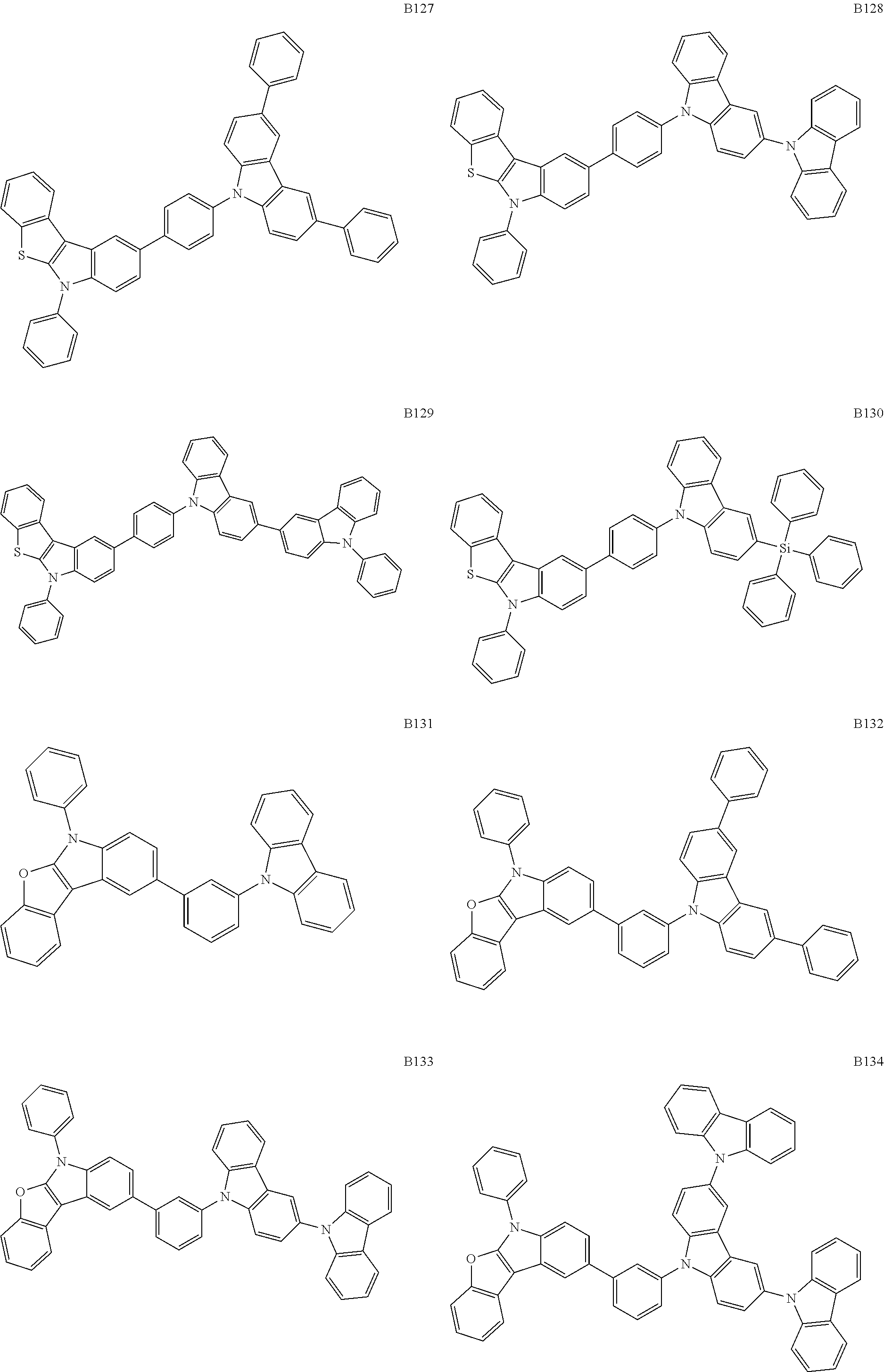 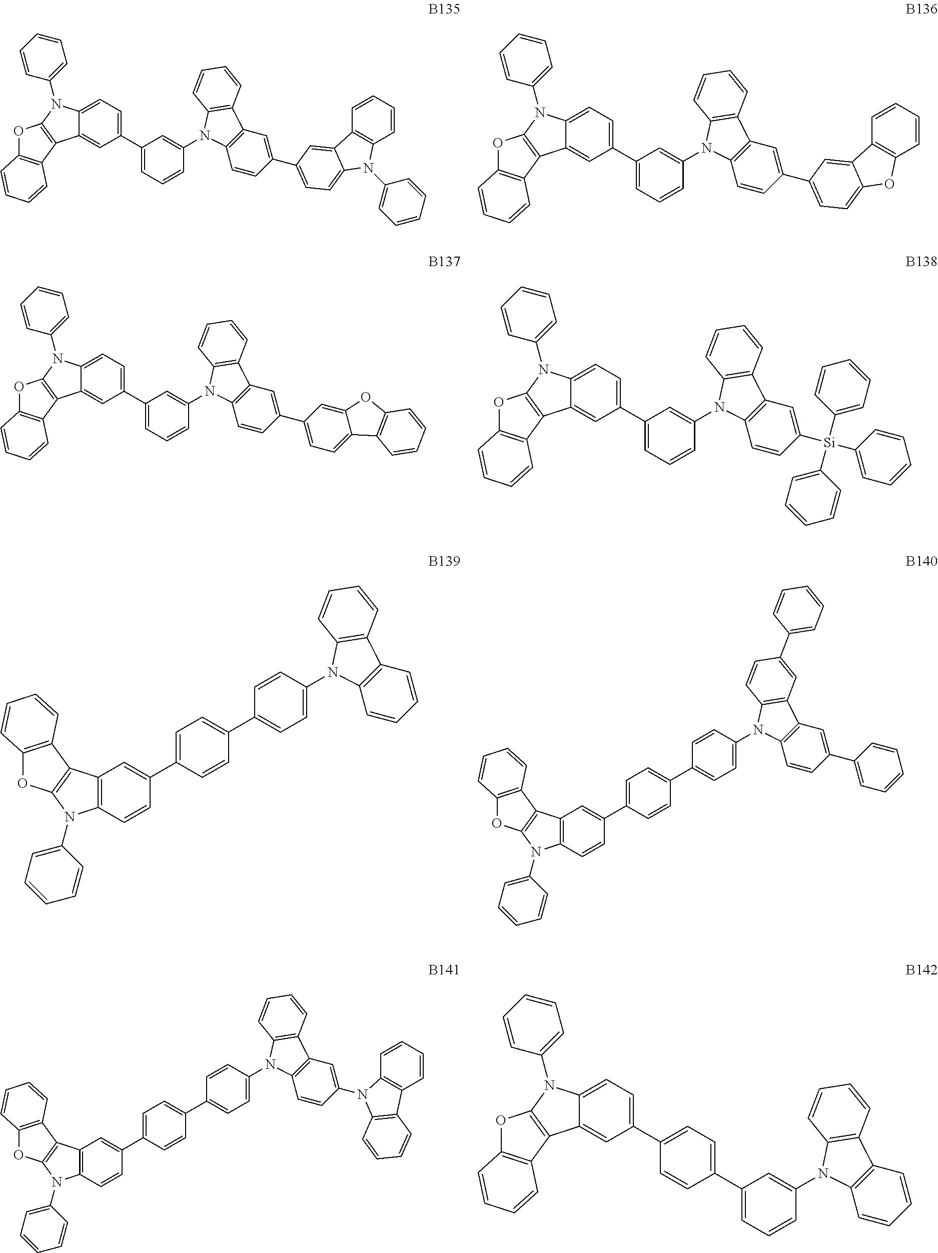 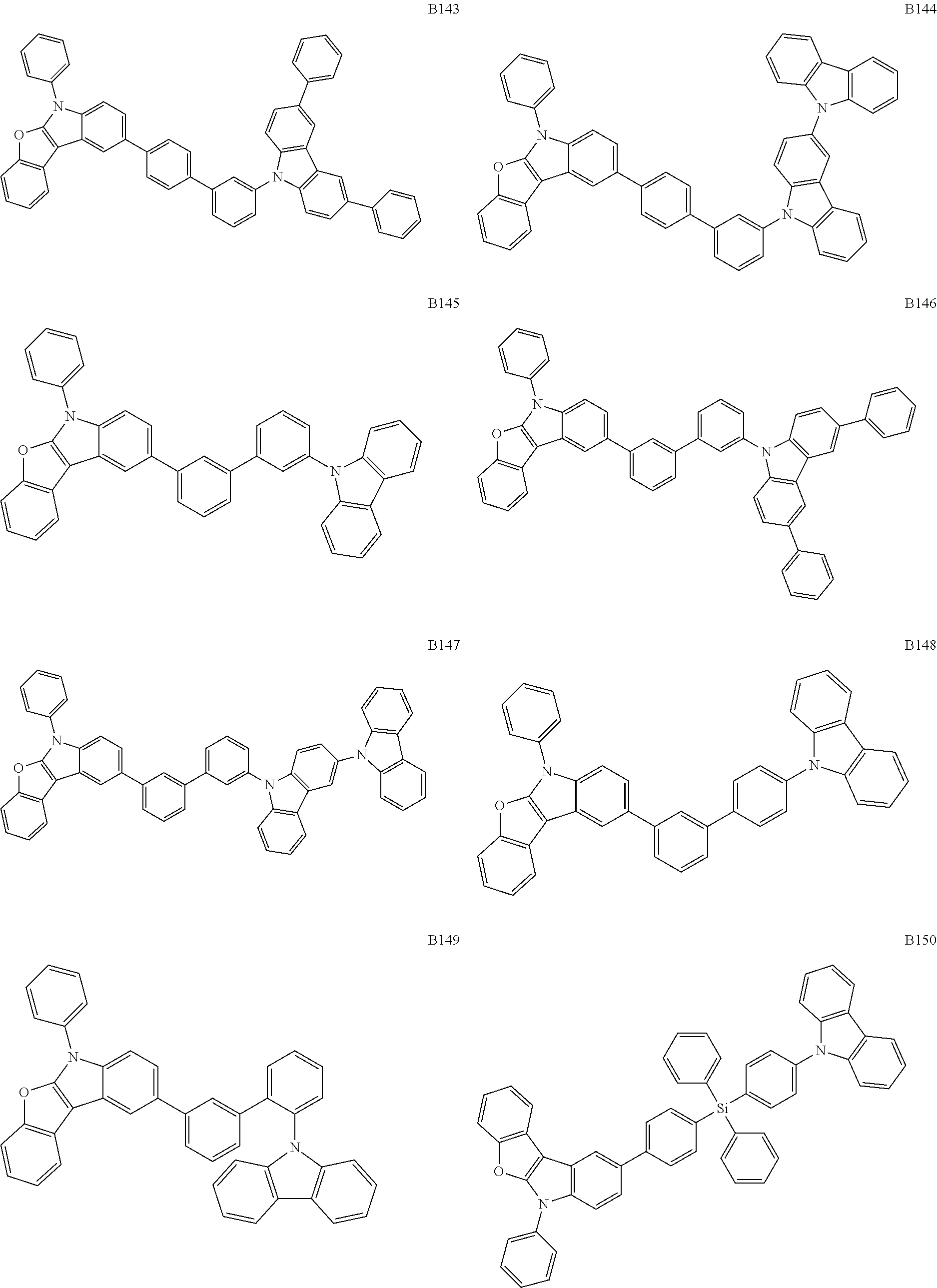 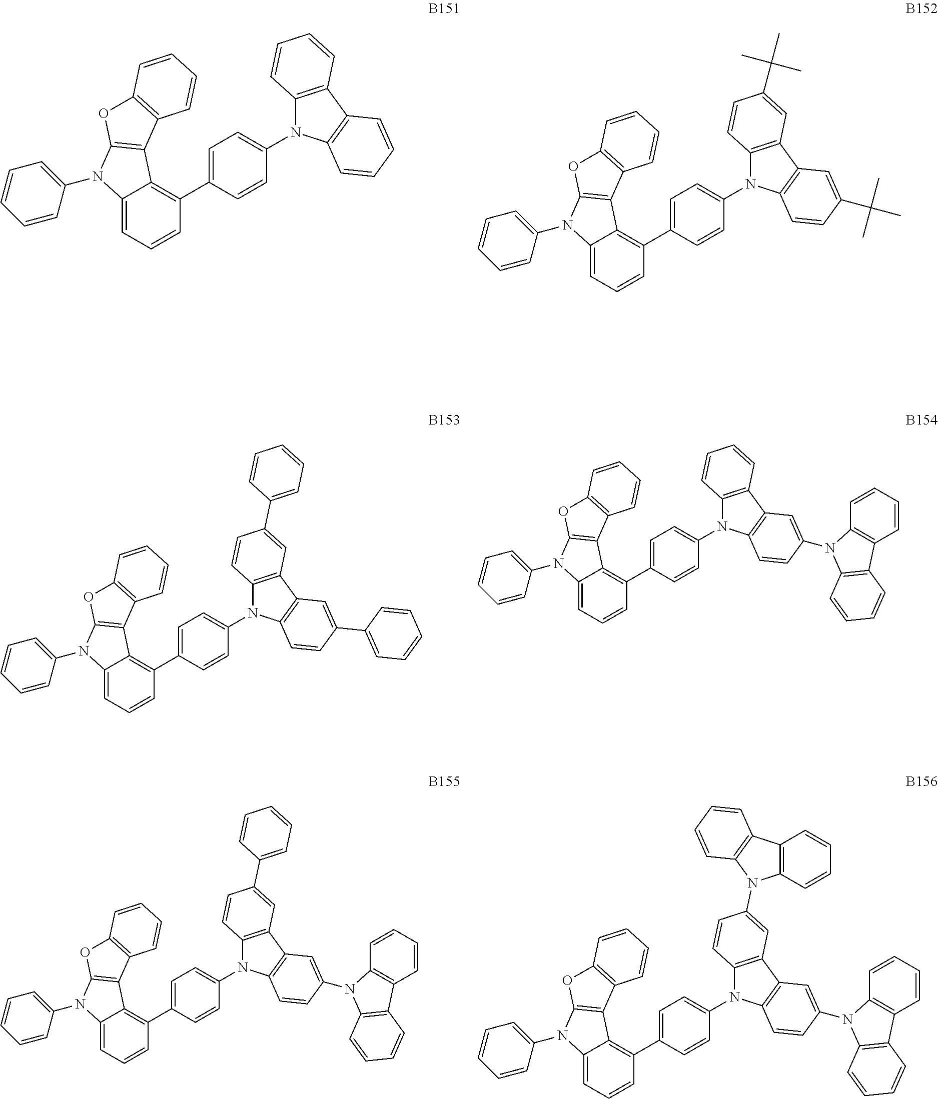 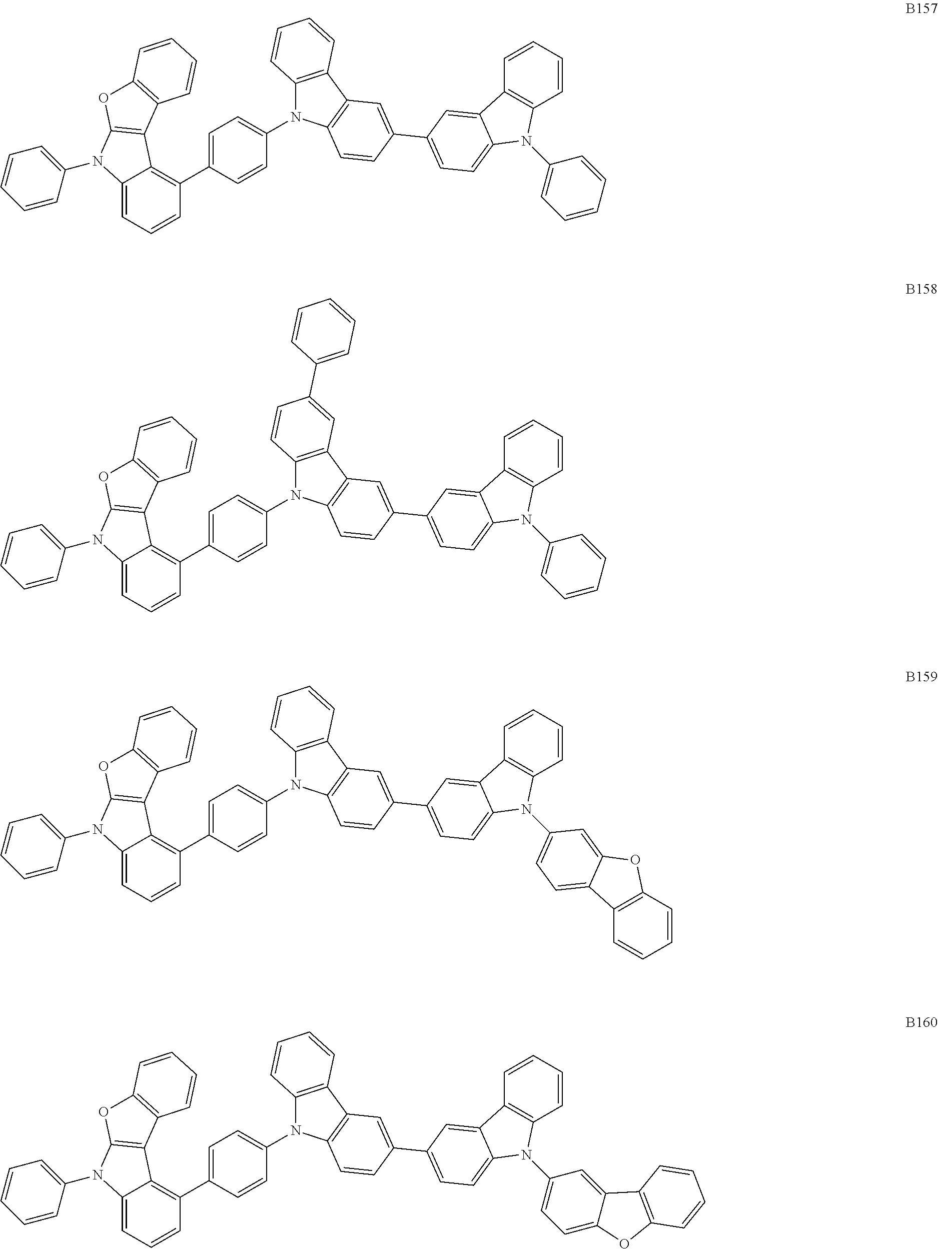 [SMILES: c1ccc(-n2c3ccccc3c3cc(-c4ccc5c(c4)c4ccccc4n5-c4ccc(-c5cccc6c5c5c7ccccc7oc5n6-c5ccccc5)cc4)ccc32)cc1 ; c1ccc(-c2ccc3c(c2)c2cc(-c4ccc5c(c4)c4ccccc4n5-c4ccccc4)ccc2n3-c2ccc(-c3cccc4c3c3c5ccccc5oc3n4-c3ccccc3)cc2)cc1 ; c1ccc(-n2c3cccc(-c4ccc(-n5c6ccccc6c6cc(-c7ccc8c(c7)c7ccccc7n8-c7ccc8c(c7)oc7ccccc78)ccc65)cc4)c3c3c4ccccc4oc32)cc1 ; c1ccc(-n2c3cccc(-c4ccc(-n5c6ccccc6c6cc(-c7ccc8c(c7)c7ccccc7n8-c7ccc8oc9ccccc9c8c7)ccc65)cc4)c3c3c4ccccc4oc32)cc1] 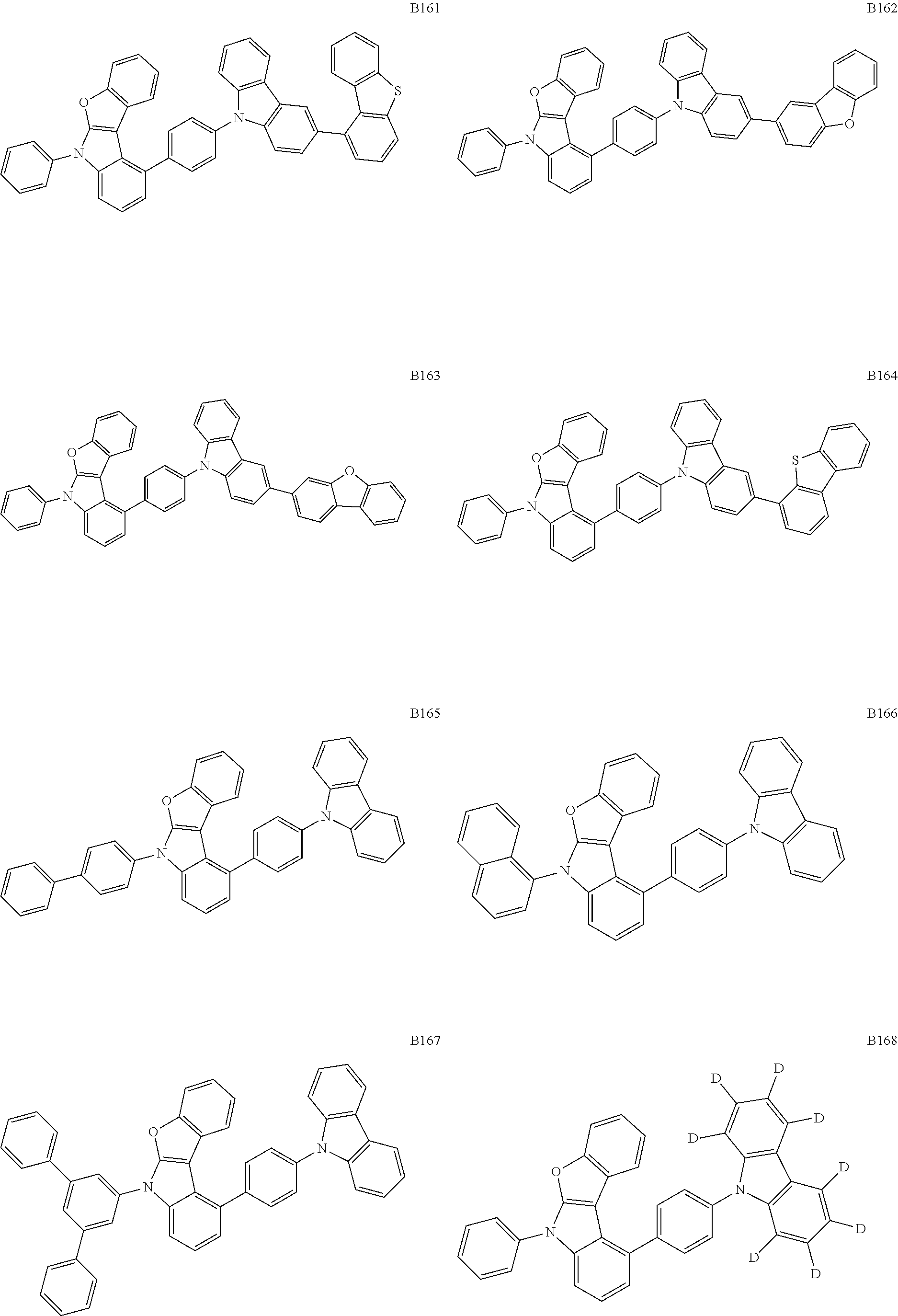 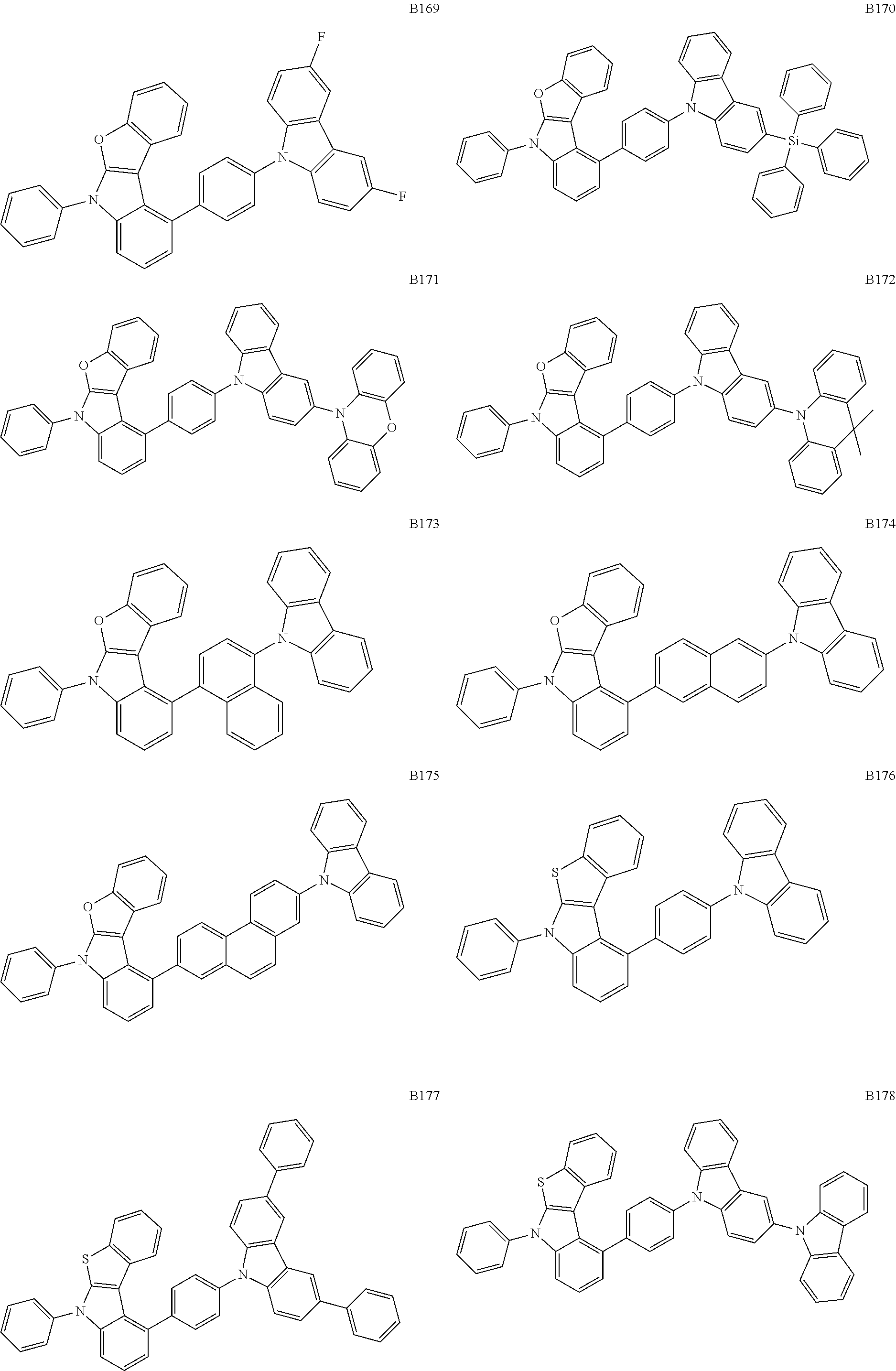 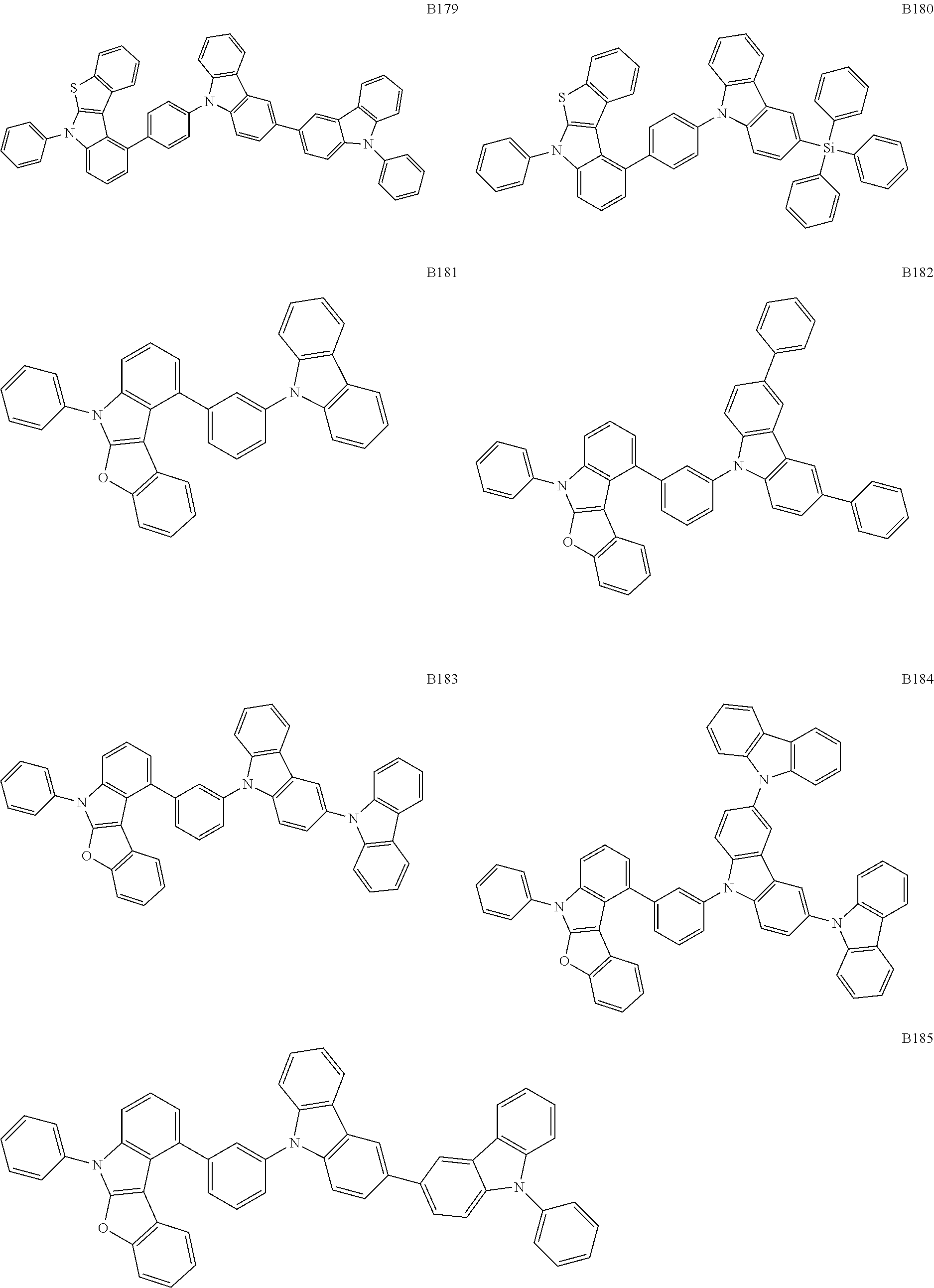 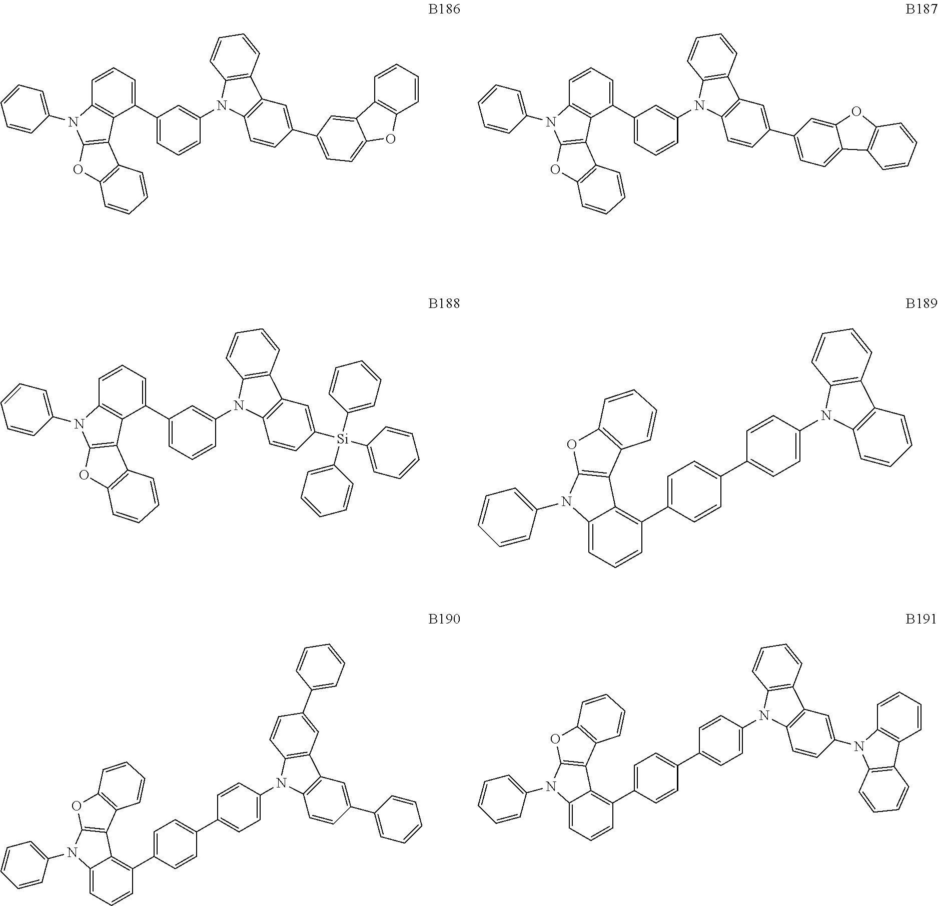 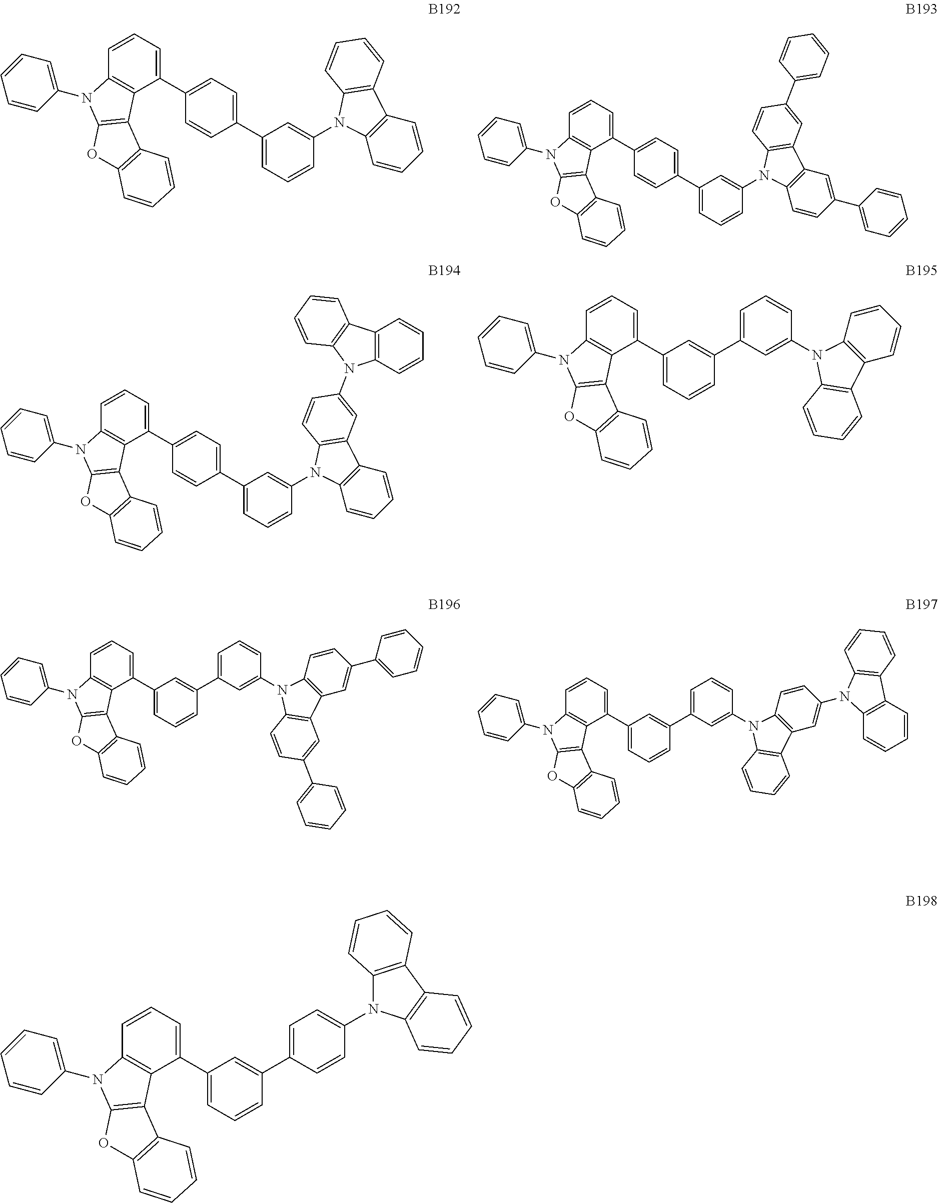 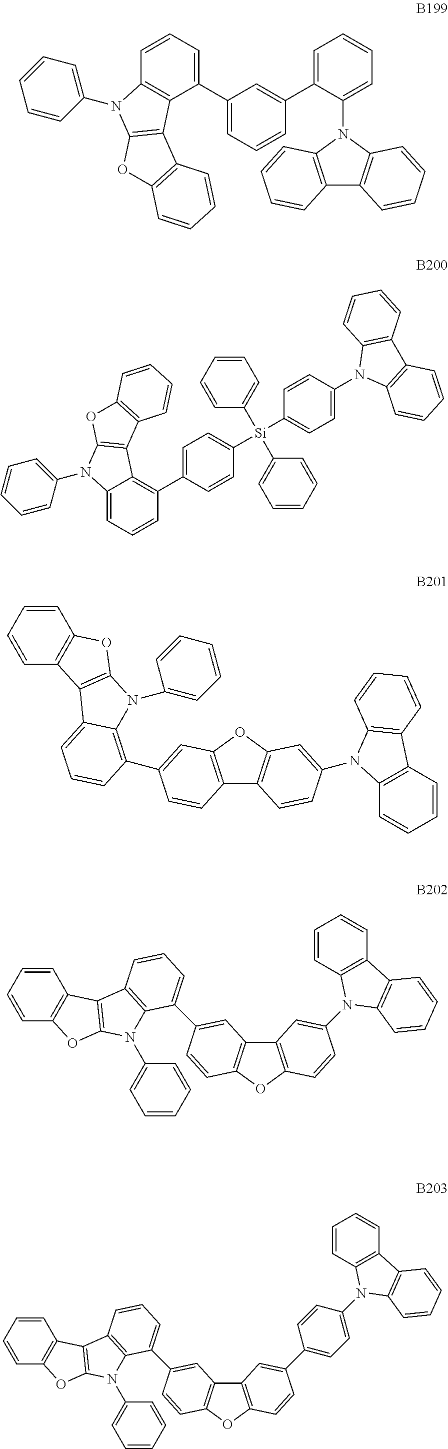 [SMILES: c1ccc(-n2c3cccc(-c4cccc(-c5ccccc5-n5c6ccccc6c6ccccc65)c4)c3c3c4ccccc4oc32)cc1 ; c1ccc(-n2c3cccc(-c4ccc([Si](c5ccccc5)(c5ccccc5)c5ccc(-n6c7ccccc7c7ccccc76)cc5)cc4)c3c3c4ccccc4oc32)cc1 ; c1ccc(-n2c3oc4ccccc4c3c3cccc(-c4ccc5c(c4)oc4cc(-n6c7ccccc7c7ccccc76)ccc45)c32)cc1 ; c1ccc(-n2c3oc4ccccc4c3c3cccc(-c4ccc5oc6ccc(-n7c8ccccc8c8ccccc87)cc6c5c4)c32)cc1 ; c1ccc(-n2c3oc4ccccc4c3c3cccc(-c4ccc5oc6ccc(-c7ccc(-n8c9ccccc9c9ccccc98)cc7)cc6c5c4)c32)cc1] 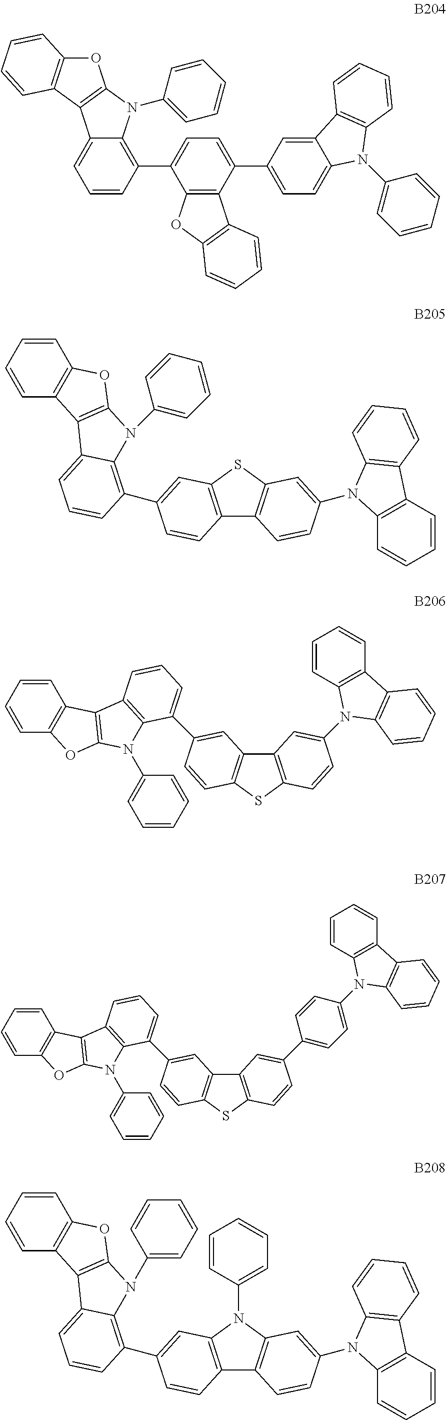 [SMILES: c1ccc(-n2c3ccccc3c3cc(-c4ccc(-c5cccc6c7c8ccccc8oc7n(-c7ccccc7)c56)c5oc6ccccc6c45)ccc32)cc1 ; c1ccc(-n2c3oc4ccccc4c3c3cccc(-c4ccc5c(c4)sc4cc(-n6c7ccccc7c7ccccc76)ccc45)c32)cc1 ; c1ccc(-n2c3oc4ccccc4c3c3cccc(-c4ccc5sc6ccc(-n7c8ccccc8c8ccccc87)cc6c5c4)c32)cc1 ; c1ccc(-n2c3oc4ccccc4c3c3cccc(-c4ccc5sc6ccc(-c7ccc(-n8c9ccccc9c9ccccc98)cc7)cc6c5c4)c32)cc1 ; c1ccc(-n2c3cc(-c4cccc5c6c7ccccc7oc6n(-c6ccccc6)c45)ccc3c3ccc(-n4c5ccccc5c5ccccc54)cc32)cc1] 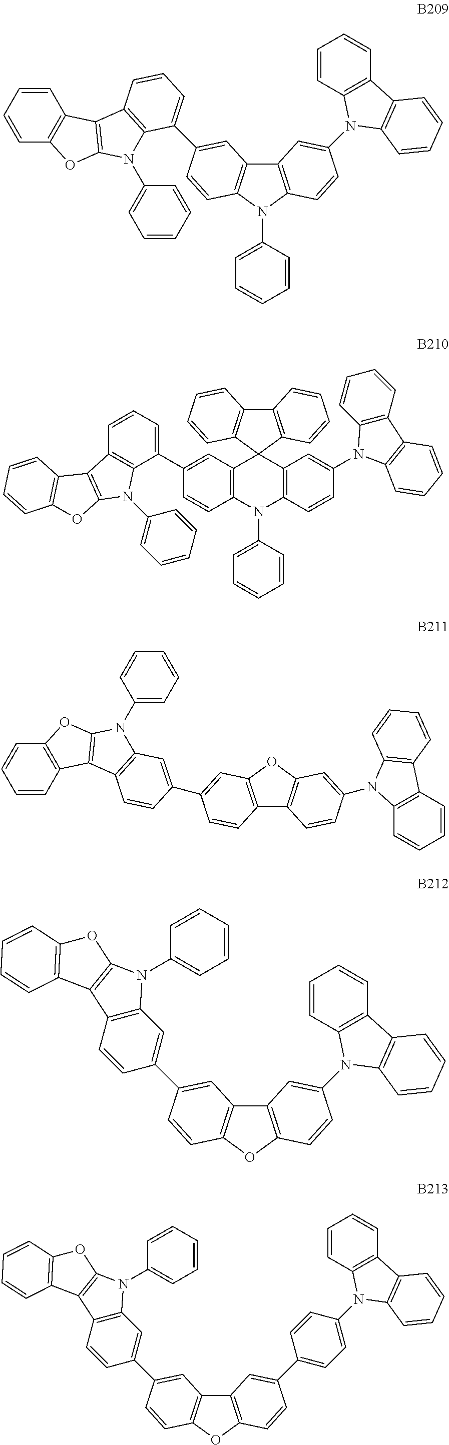 [SMILES: c1ccc(-n2c3ccc(-c4cccc5c6c7ccccc7oc6n(-c6ccccc6)c45)cc3c3cc(-n4c5ccccc5c5ccccc54)ccc32)cc1 ; c1ccc(N2c3ccc(-c4cccc5c6c7ccccc7oc6n(-c6ccccc6)c45)cc3C3(c4ccccc4-c4ccccc43)c3cc(-n4c5ccccc5c5ccccc54)ccc32)cc1 ; c1ccc(-n2c3cc(-c4ccc5c(c4)oc4cc(-n6c7ccccc7c7ccccc76)ccc45)ccc3c3c4ccccc4oc32)cc1 ; c1ccc(-n2c3cc(-c4ccc5oc6ccc(-n7c8ccccc8c8ccccc87)cc6c5c4)ccc3c3c4ccccc4oc32)cc1 ; c1ccc(-n2c3cc(-c4ccc5oc6ccc(-c7ccc(-n8c9ccccc9c9ccccc98)cc7)cc6c5c4)ccc3c3c4ccccc4oc32)cc1] 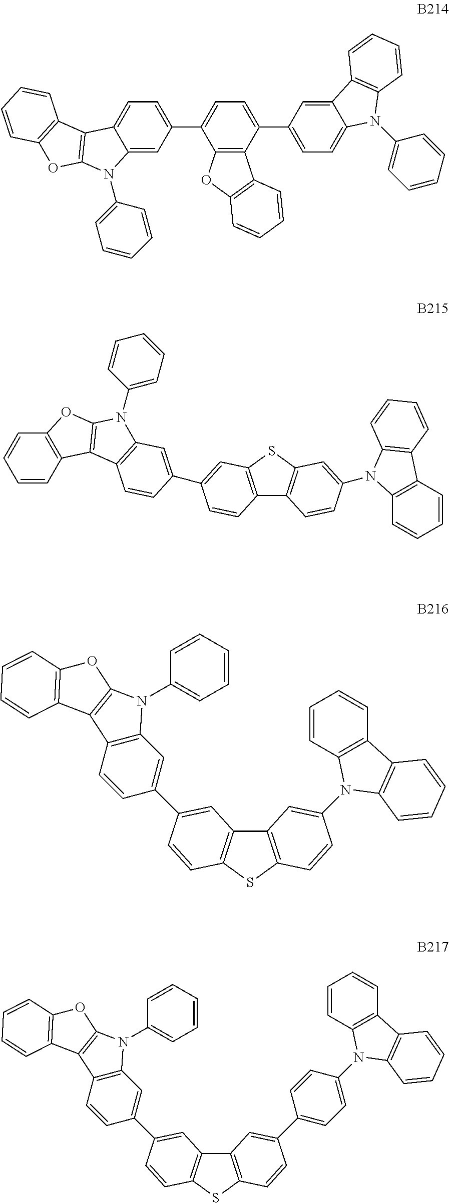 [SMILES: c1ccc(-n2c3ccccc3c3cc(-c4ccc(-c5ccc6c7c8ccccc8oc7n(-c7ccccc7)c6c5)c5oc6ccccc6c45)ccc32)cc1 ; c1ccc(-n2c3cc(-c4ccc5c(c4)sc4cc(-n6c7ccccc7c7ccccc76)ccc45)ccc3c3c4ccccc4oc32)cc1 ; c1ccc(-n2c3cc(-c4ccc5sc6ccc(-n7c8ccccc8c8ccccc87)cc6c5c4)ccc3c3c4ccccc4oc32)cc1 ; c1ccc(-n2c3cc(-c4ccc5sc6ccc(-c7ccc(-n8c9ccccc9c9ccccc98)cc7)cc6c5c4)ccc3c3c4ccccc4oc32)cc1] 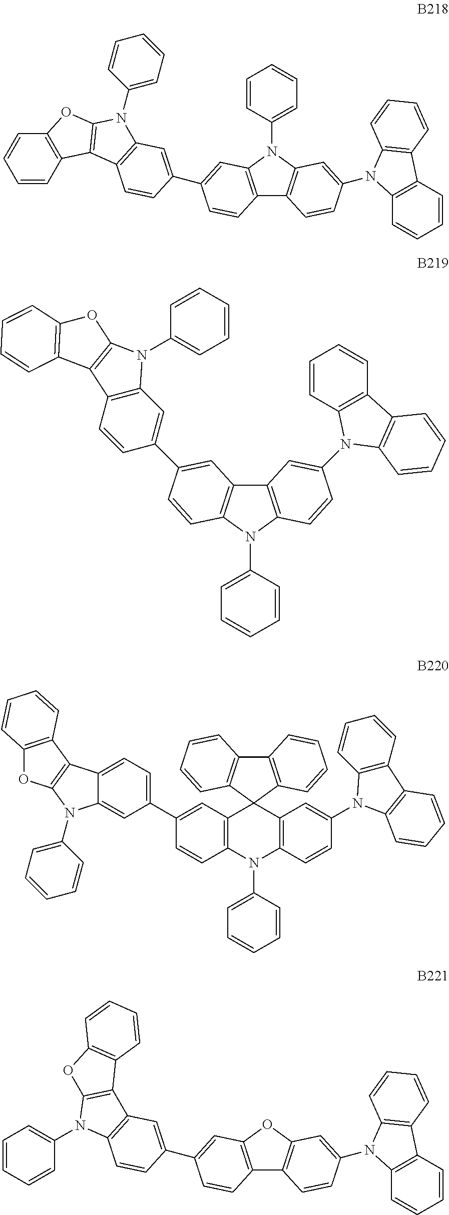 [SMILES: c1ccc(-n2c3cc(-c4ccc5c6c7ccccc7oc6n(-c6ccccc6)c5c4)ccc3c3ccc(-n4c5ccccc5c5ccccc54)cc32)cc1 ; c1ccc(-n2c3ccc(-c4ccc5c6c7ccccc7oc6n(-c6ccccc6)c5c4)cc3c3cc(-n4c5ccccc5c5ccccc54)ccc32)cc1 ; c1ccc(N2c3ccc(-c4ccc5c6c7ccccc7oc6n(-c6ccccc6)c5c4)cc3C3(c4ccccc4-c4ccccc43)c3cc(-n4c5ccccc5c5ccccc54)ccc32)cc1 ; c1ccc(-n2c3ccc(-c4ccc5c(c4)oc4cc(-n6c7ccccc7c7ccccc76)ccc45)cc3c3c4ccccc4oc32)cc1] 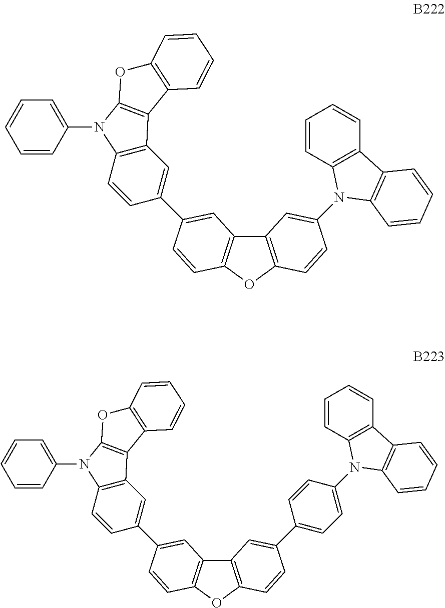 [SMILES: c1ccc(-n2c3ccc(-c4ccc5oc6ccc(-n7c8ccccc8c8ccccc87)cc6c5c4)cc3c3c4ccccc4oc32)cc1 ; c1ccc(-n2c3ccc(-c4ccc5oc6ccc(-c7ccc(-n8c9ccccc9c9ccccc98)cc7)cc6c5c4)cc3c3c4ccccc4oc32)cc1] 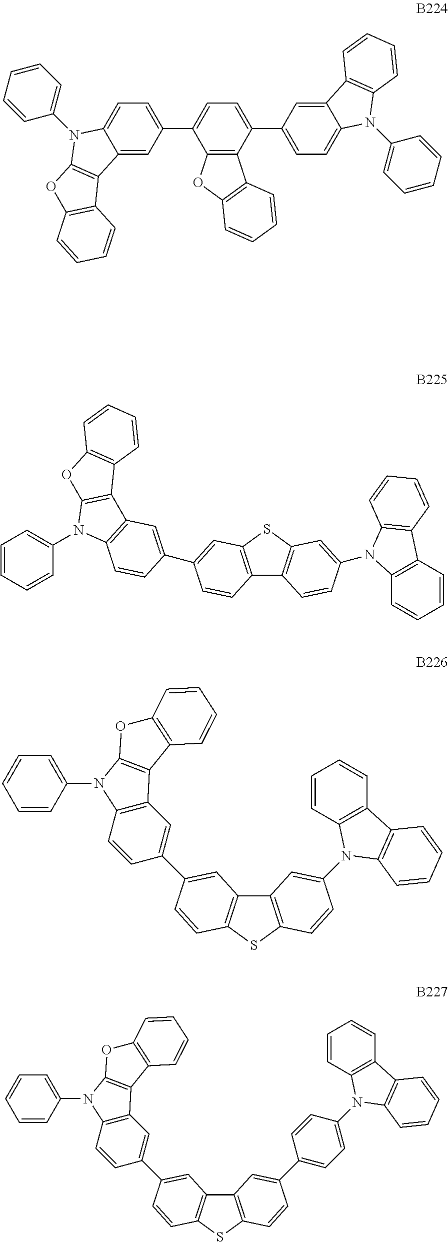 [SMILES: c1ccc(-n2c3ccccc3c3cc(-c4ccc(-c5ccc6c(c5)c5c7ccccc7oc5n6-c5ccccc5)c5oc6ccccc6c45)ccc32)cc1 ; c1ccc(-n2c3ccc(-c4ccc5c(c4)sc4cc(-n6c7ccccc7c7ccccc76)ccc45)cc3c3c4ccccc4oc32)cc1 ; c1ccc(-n2c3ccc(-c4ccc5sc6ccc(-n7c8ccccc8c8ccccc87)cc6c5c4)cc3c3c4ccccc4oc32)cc1 ; c1ccc(-n2c3ccc(-c4ccc5sc6ccc(-c7ccc(-n8c9ccccc9c9ccccc98)cc7)cc6c5c4)cc3c3c4ccccc4oc32)cc1] 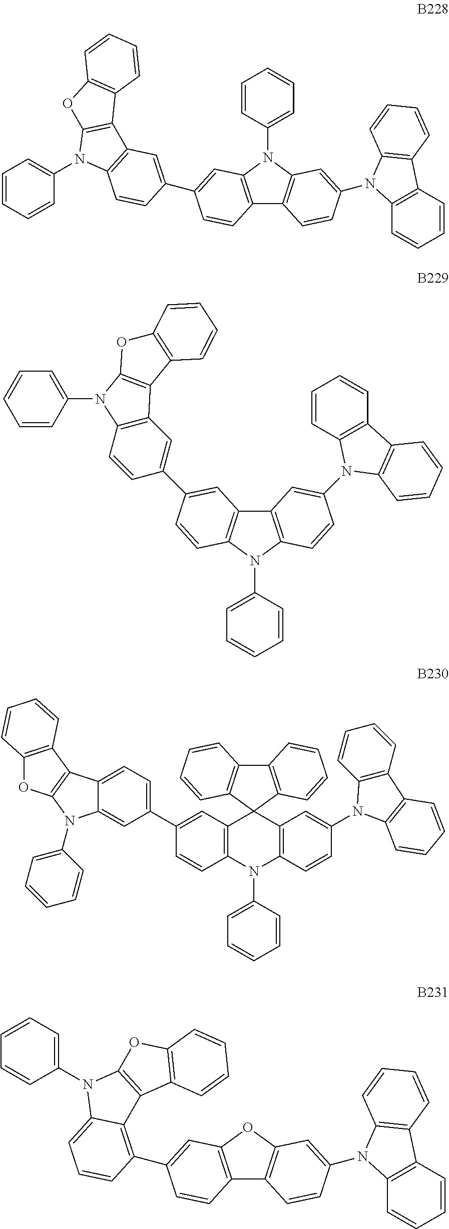 [SMILES: c1ccc(-n2c3cc(-c4ccc5c(c4)c4c6ccccc6oc4n5-c4ccccc4)ccc3c3ccc(-n4c5ccccc5c5ccccc54)cc32)cc1 ; c1ccc(-n2c3ccc(-c4ccc5c(c4)c4c6ccccc6oc4n5-c4ccccc4)cc3c3cc(-n4c5ccccc5c5ccccc54)ccc32)cc1 ; c1ccc(N2c3ccc(-c4ccc5c6c7ccccc7oc6n(-c6ccccc6)c5c4)cc3C3(c4ccccc4-c4ccccc43)c3cc(-n4c5ccccc5c5ccccc54)ccc32)cc1 ; c1ccc(-n2c3cccc(-c4ccc5c(c4)oc4cc(-n6c7ccccc7c7ccccc76)ccc45)c3c3c4ccccc4oc32)cc1] 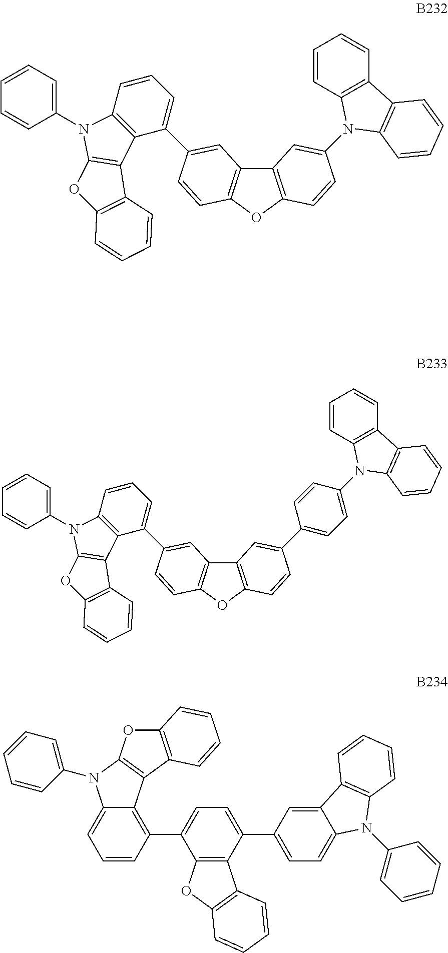 [SMILES: c1ccc(-n2c3cccc(-c4ccc5oc6ccc(-n7c8ccccc8c8ccccc87)cc6c5c4)c3c3c4ccccc4oc32)cc1 ; c1ccc(-n2c3cccc(-c4ccc5oc6ccc(-c7ccc(-n8c9ccccc9c9ccccc98)cc7)cc6c5c4)c3c3c4ccccc4oc32)cc1 ; c1ccc(-n2c3ccccc3c3cc(-c4ccc(-c5cccc6c5c5c7ccccc7oc5n6-c5ccccc5)c5oc6ccccc6c45)ccc32)cc1] 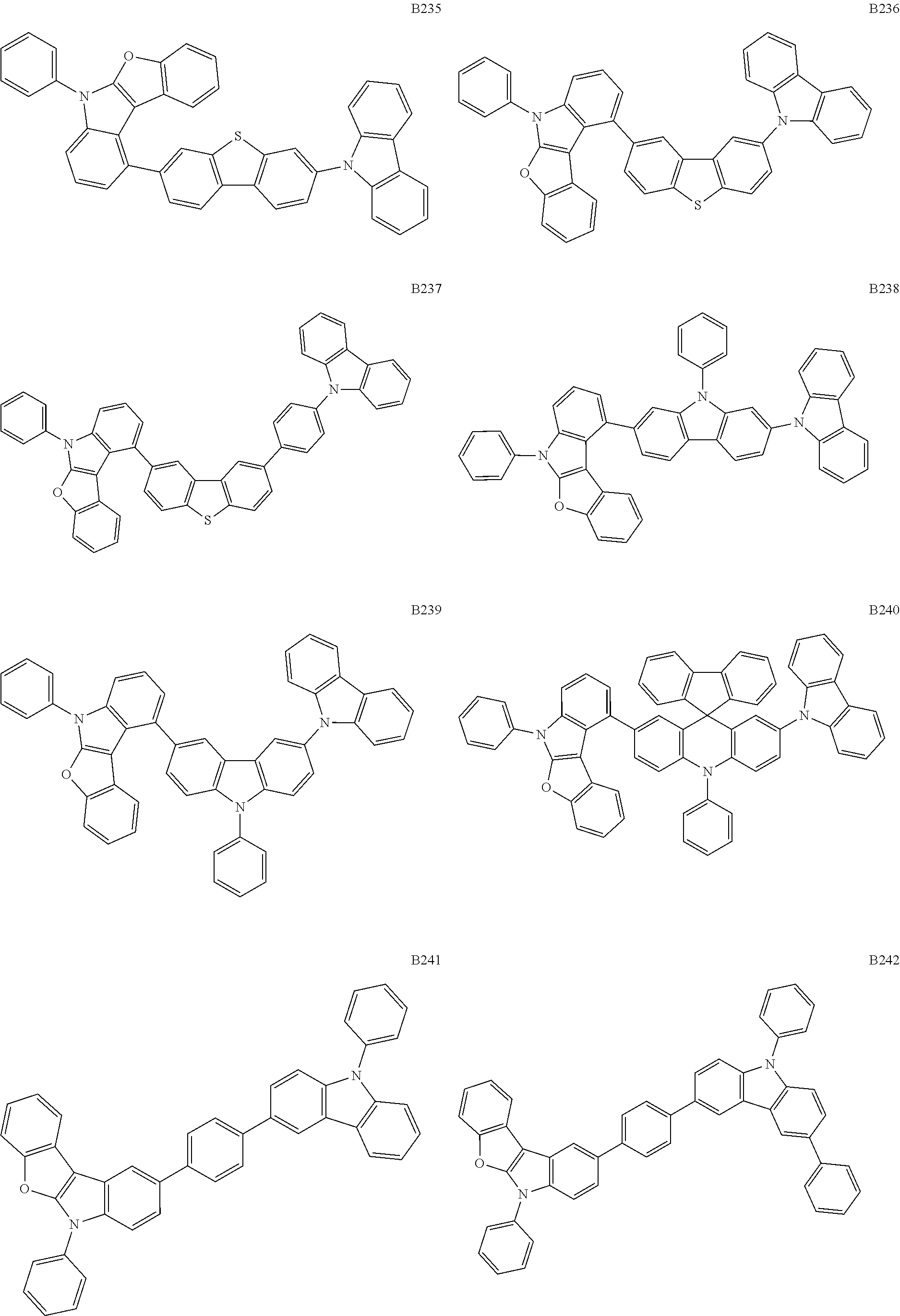 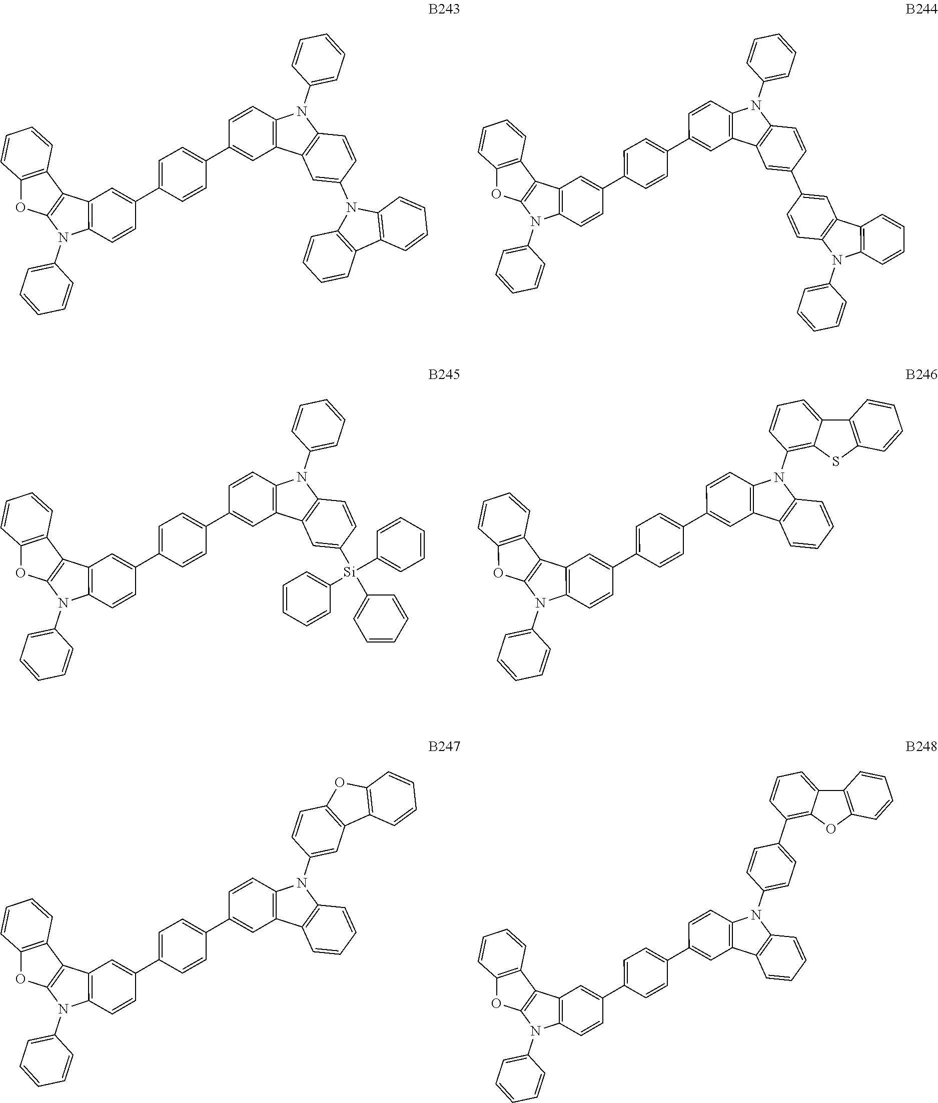 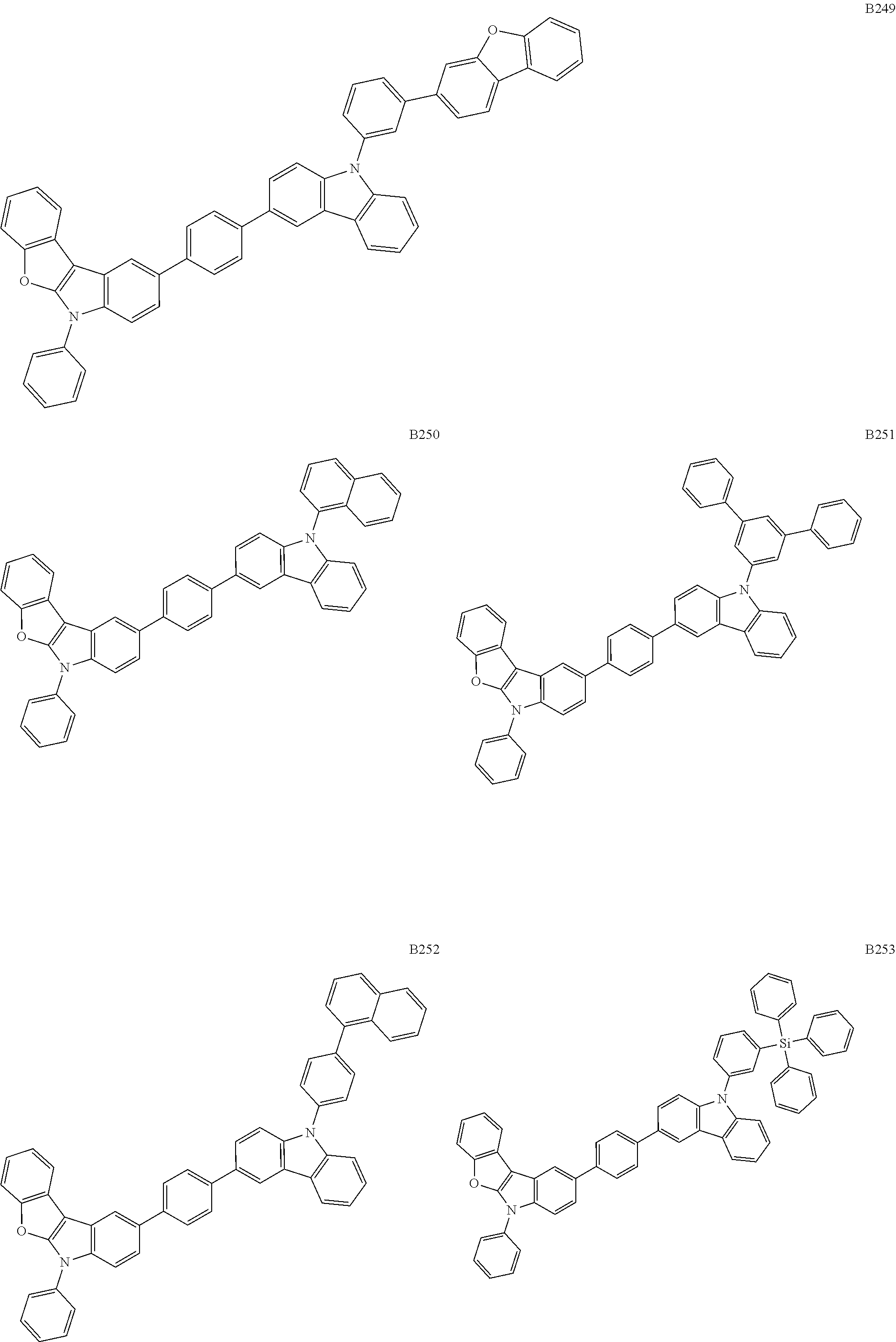 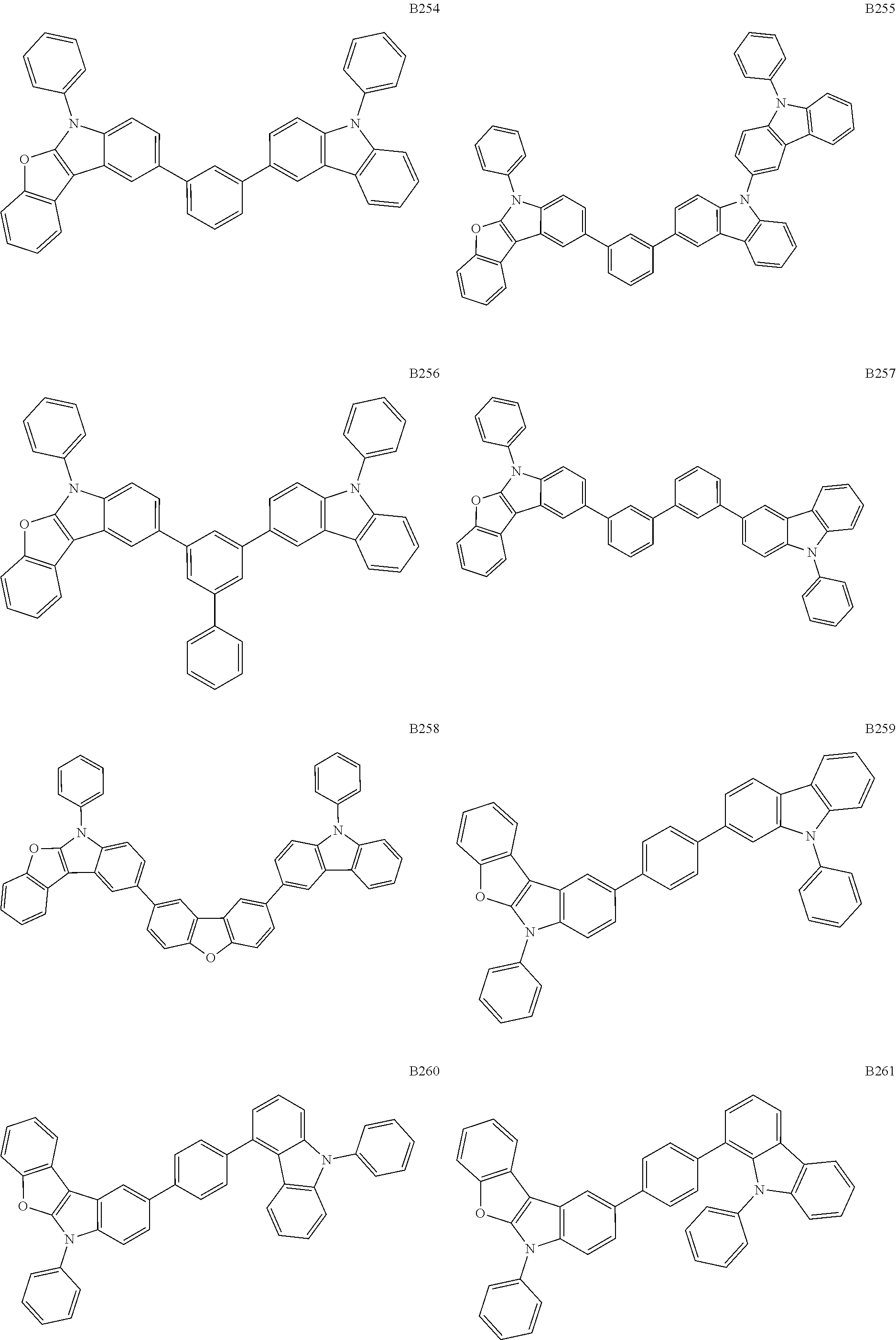 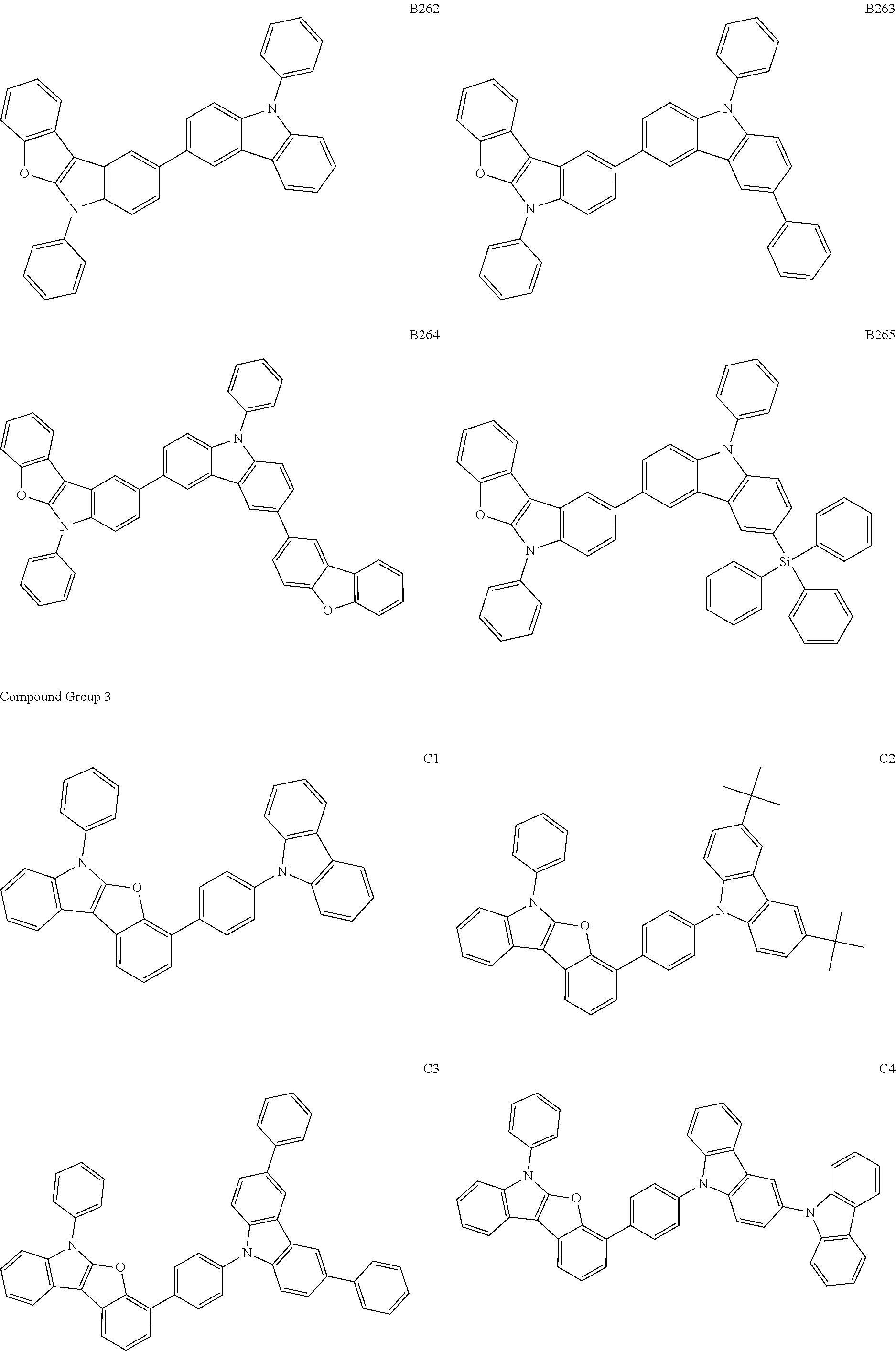 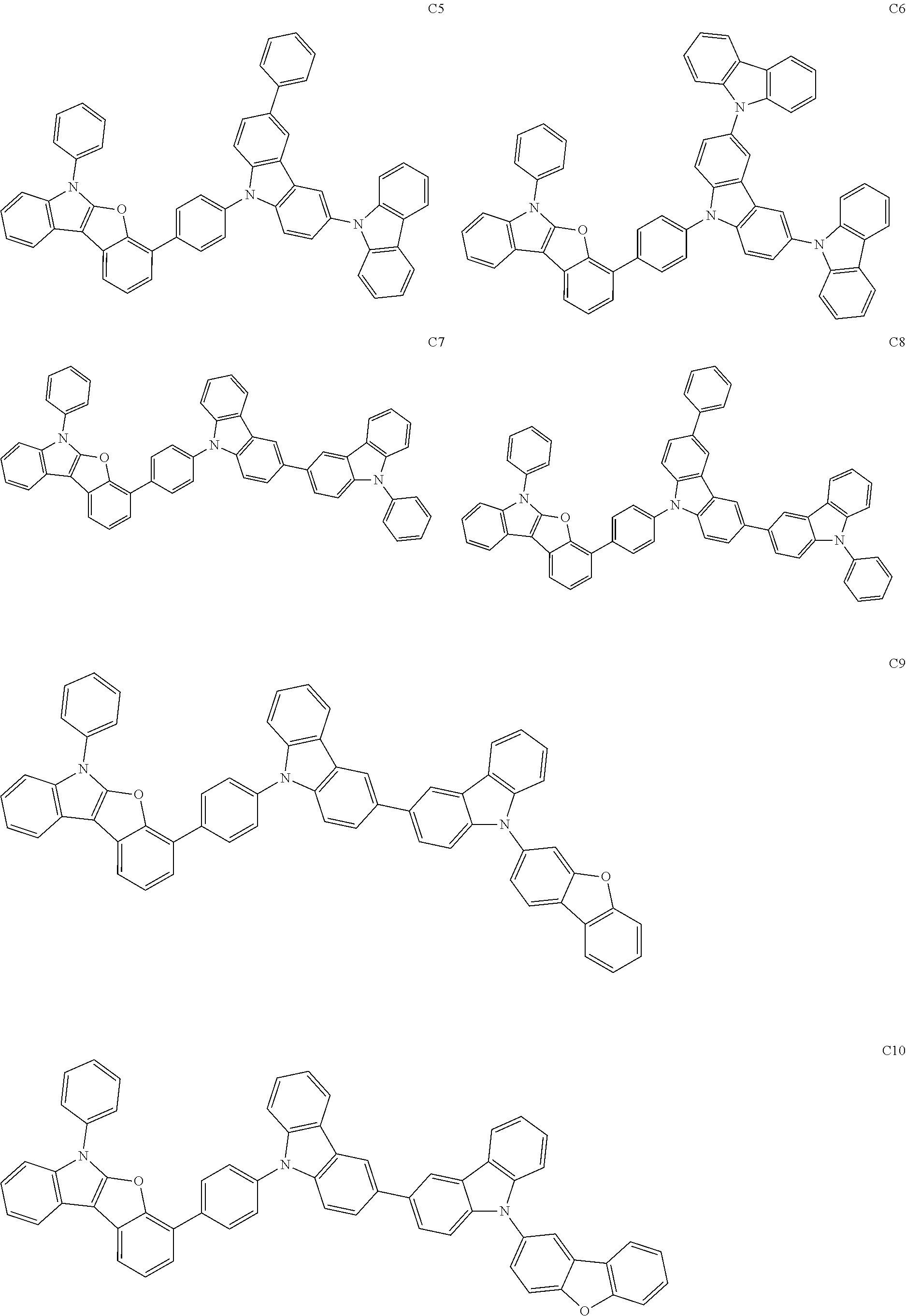 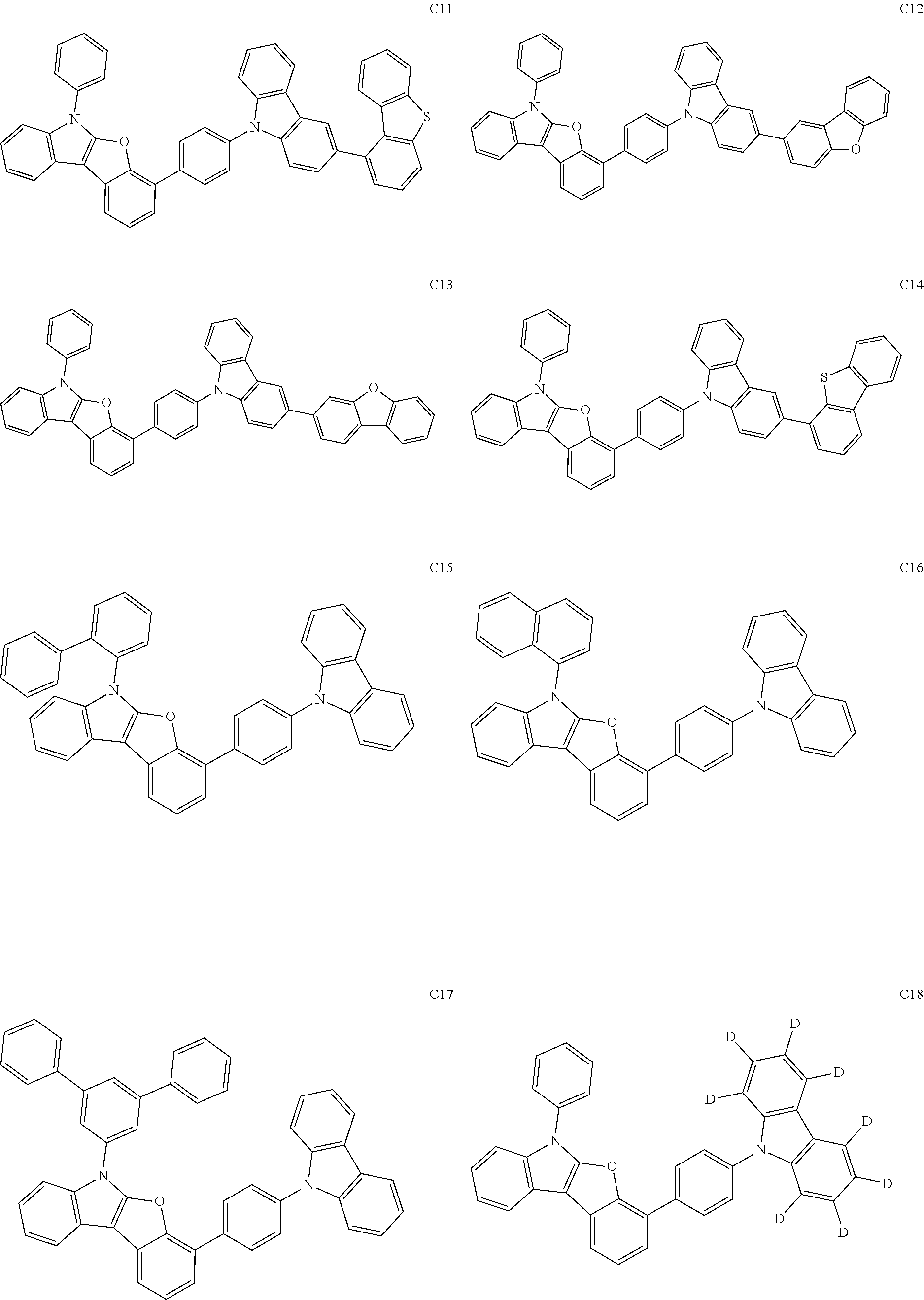 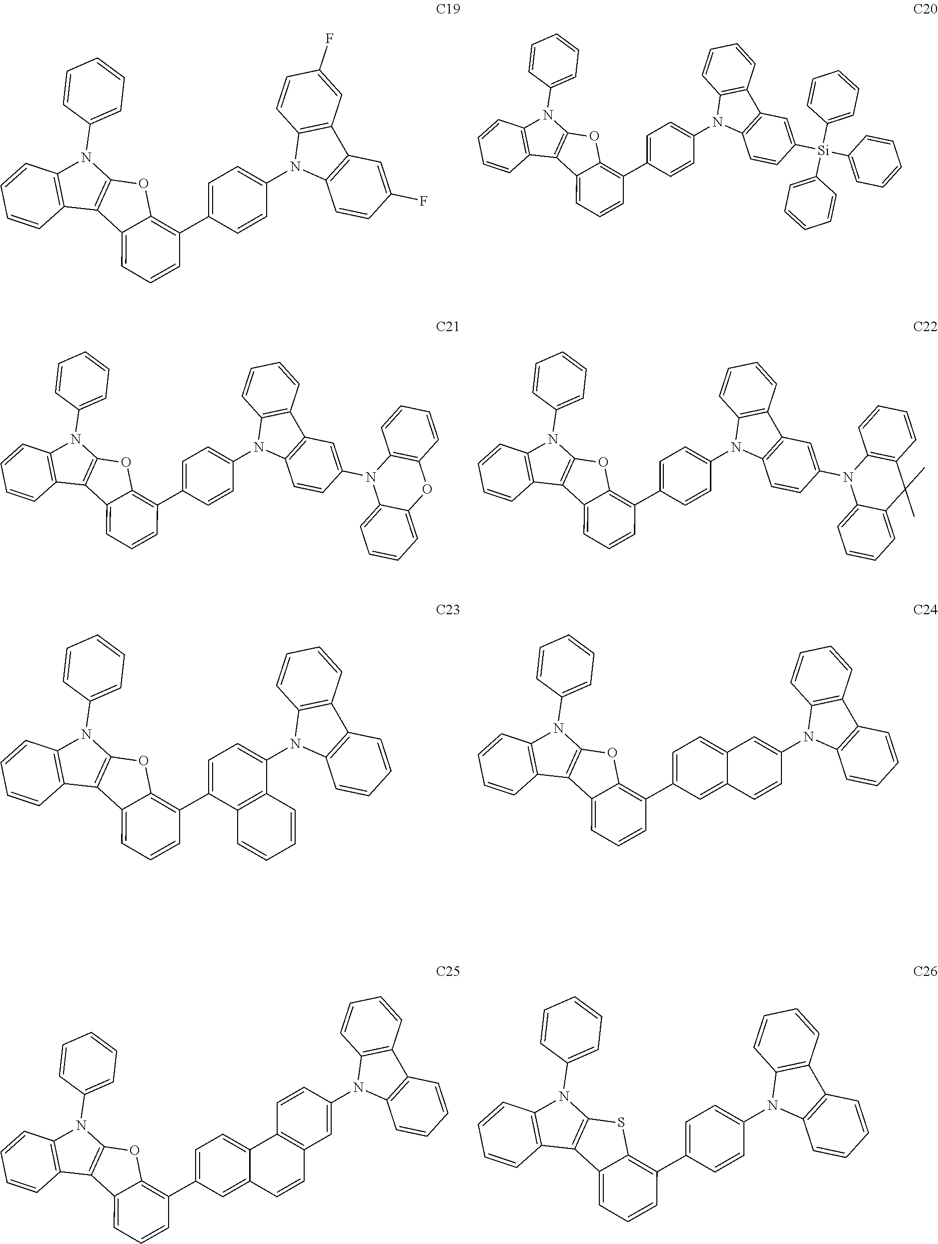 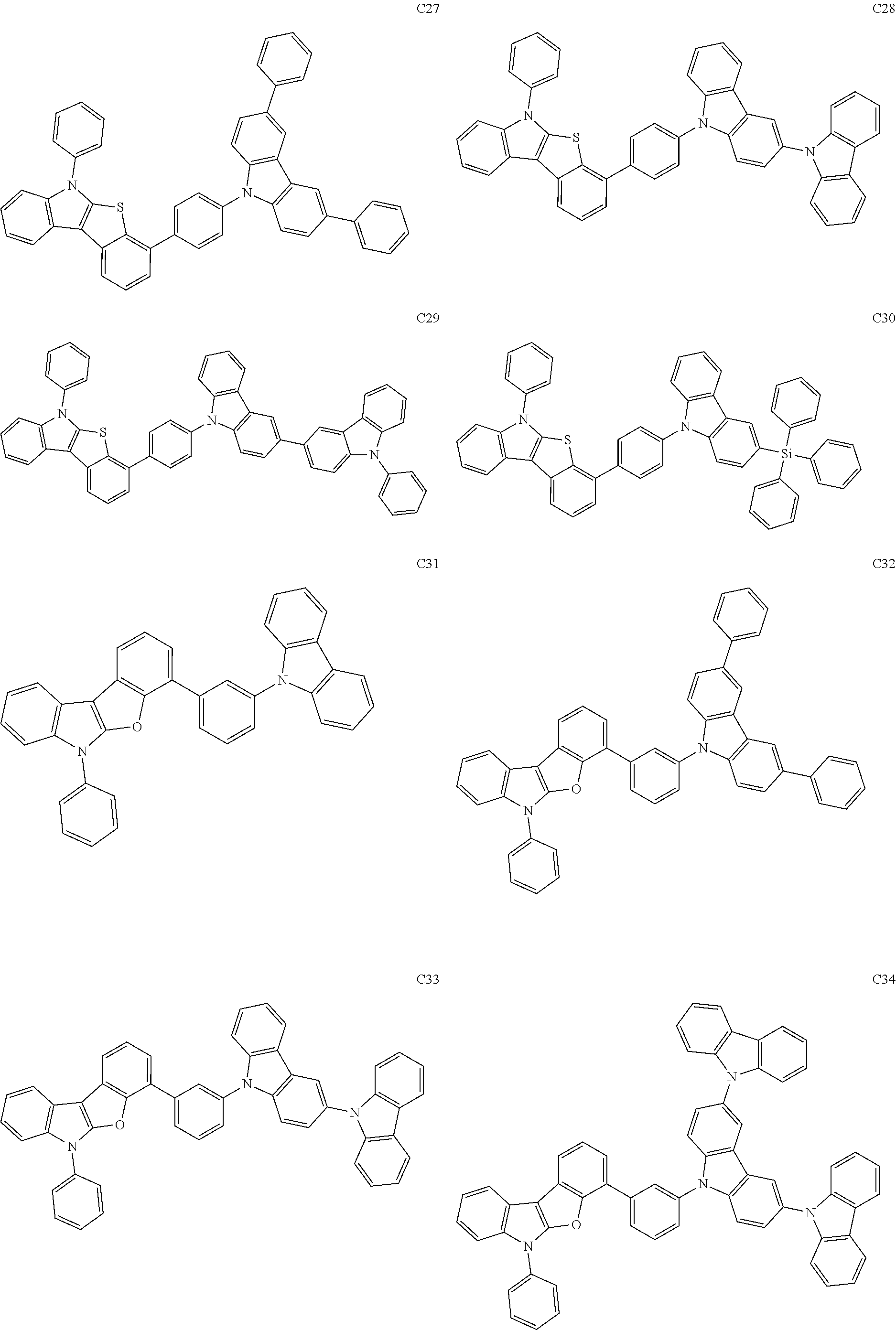 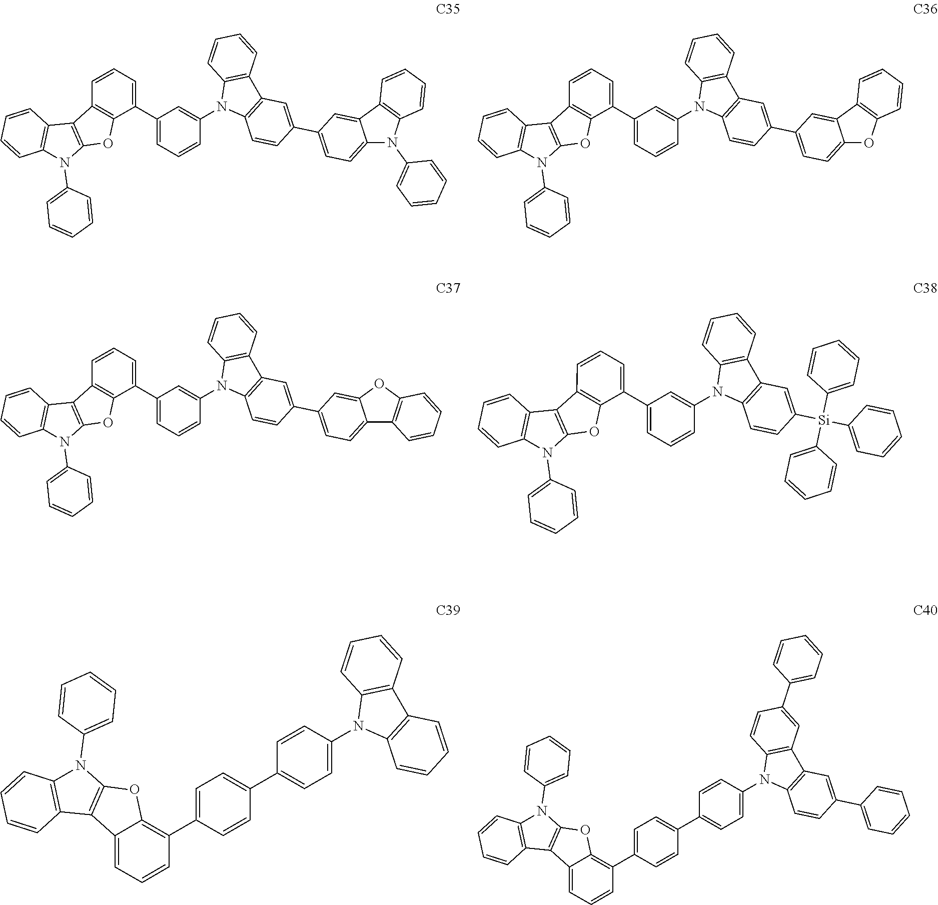 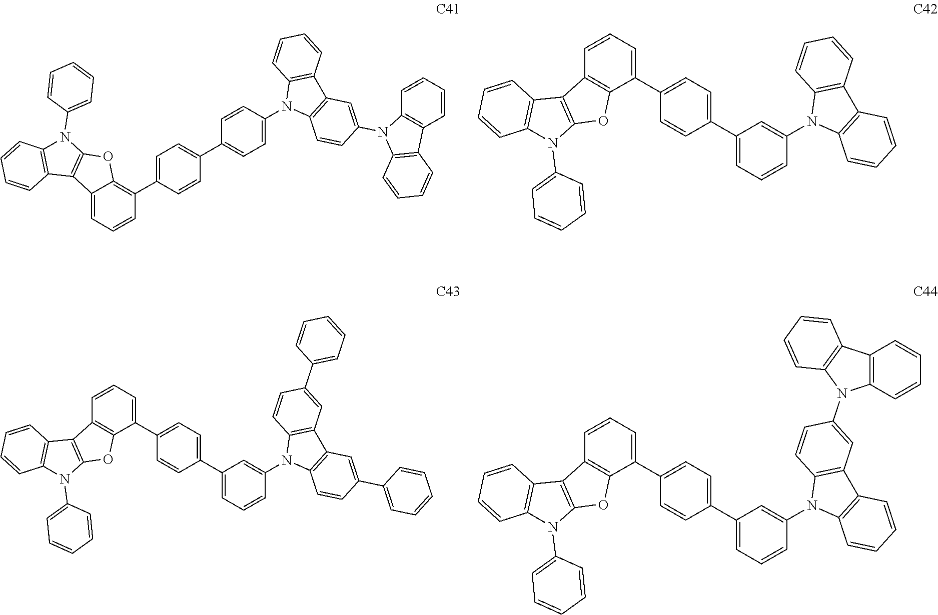 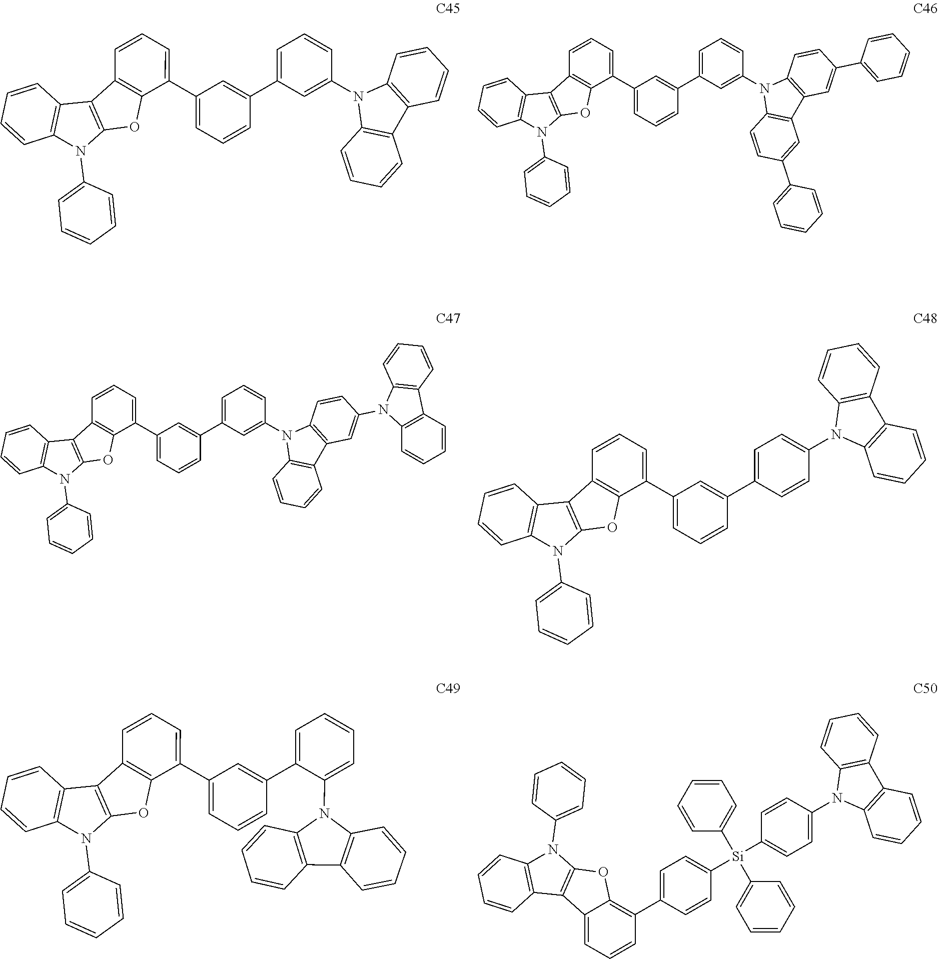 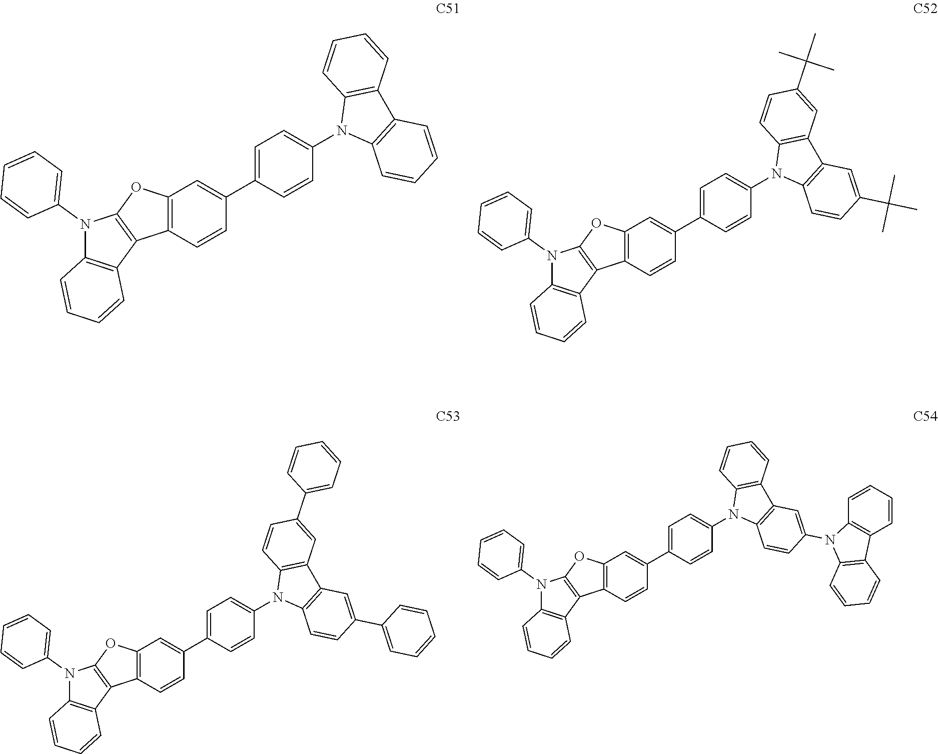 [SMILES: c1ccc(-n2c3ccccc3c3c4ccc(-c5ccc(-n6c7ccccc7c7ccccc76)cc5)cc4oc32)cc1 ; CC(C)(C)c1ccc2c(c1)c1cc(C(C)(C)C)ccc1n2-c1ccc(-c2ccc3c(c2)oc2c3c3ccccc3n2-c2ccccc2)cc1 ; c1ccc(-c2ccc3c(c2)c2cc(-c4ccccc4)ccc2n3-c2ccc(-c3ccc4c(c3)oc3c4c4ccccc4n3-c3ccccc3)cc2)cc1 ; c1ccc(-n2c3ccccc3c3c4ccc(-c5ccc(-n6c7ccccc7c7cc(-n8c9ccccc9c9ccccc98)ccc76)cc5)cc4oc32)cc1] 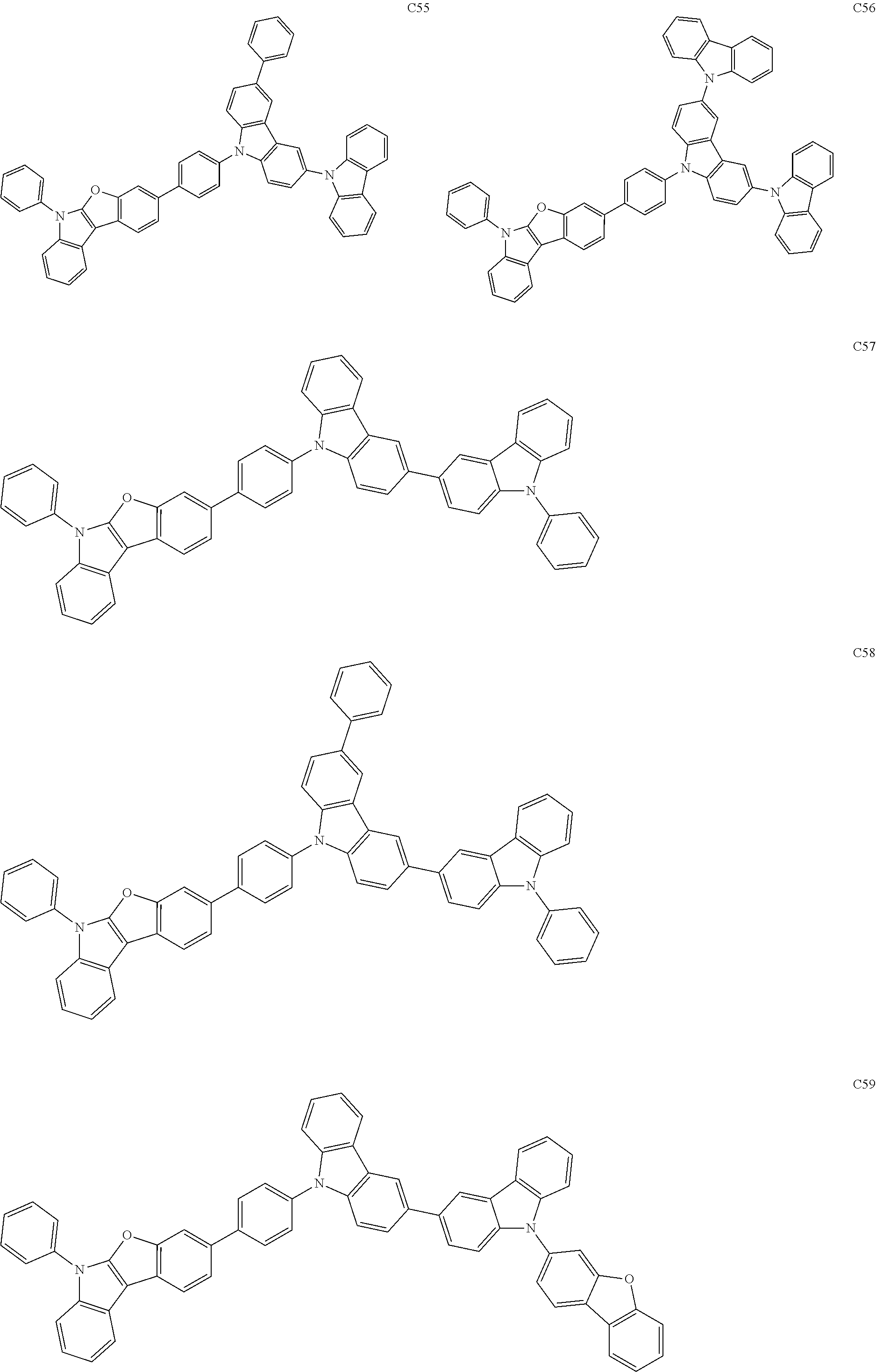 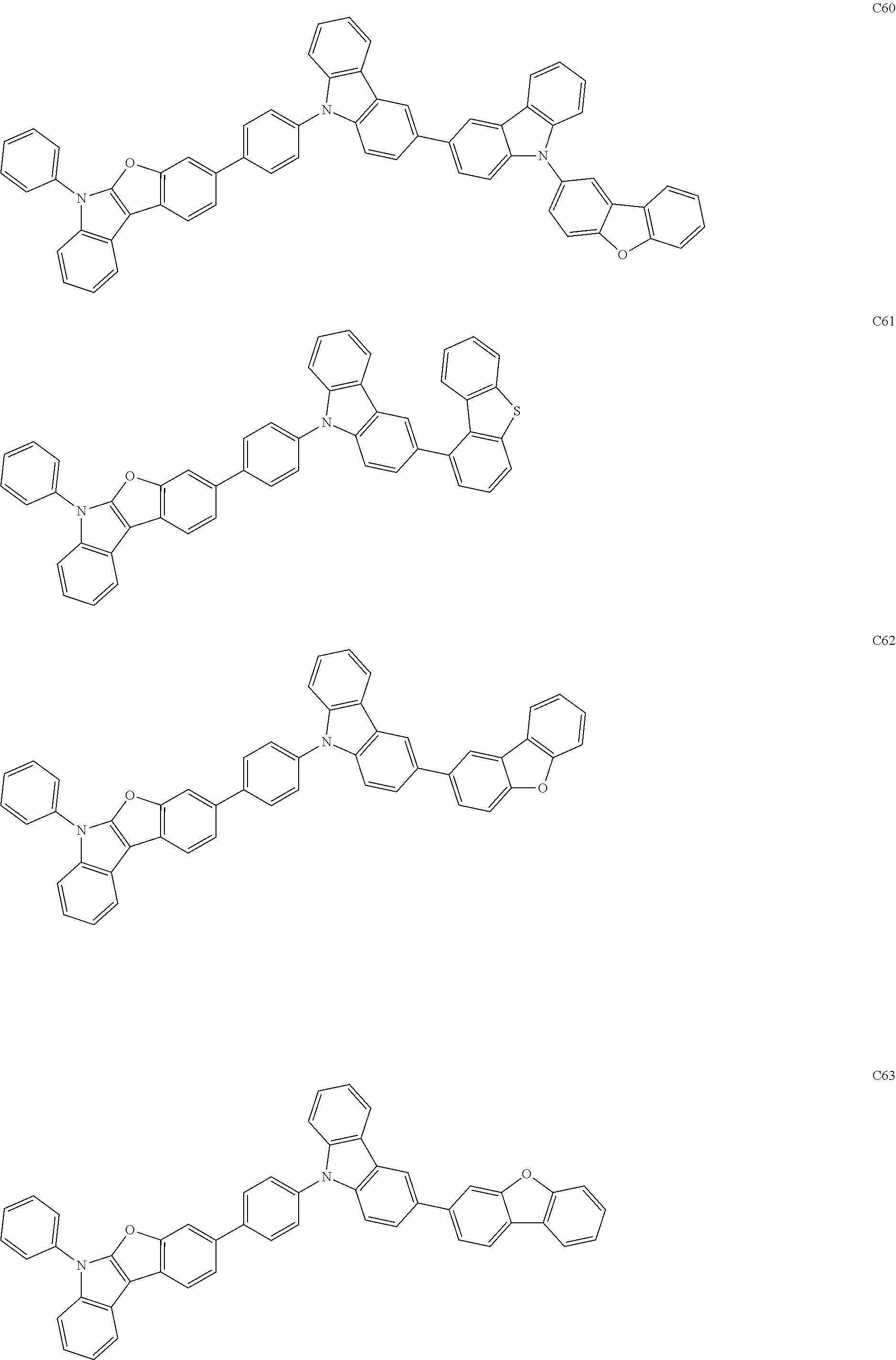 [SMILES: c1ccc(-n2c3ccccc3c3c4ccc(-c5ccc(-n6c7ccccc7c7cc(-c8ccc9c(c8)c8ccccc8n9-c8ccc9oc%10ccccc%10c9c8)ccc76)cc5)cc4oc32)cc1 ; c1ccc(-n2c3ccccc3c3c4ccc(-c5ccc(-n6c7ccccc7c7cc(-c8cccc9sc%10ccccc%10c89)ccc76)cc5)cc4oc32)cc1 ; c1ccc(-n2c3ccccc3c3c4ccc(-c5ccc(-n6c7ccccc7c7cc(-c8ccc9oc%10ccccc%10c9c8)ccc76)cc5)cc4oc32)cc1 ; c1ccc(-n2c3ccccc3c3c4ccc(-c5ccc(-n6c7ccccc7c7cc(-c8ccc9c(c8)oc8ccccc89)ccc76)cc5)cc4oc32)cc1] 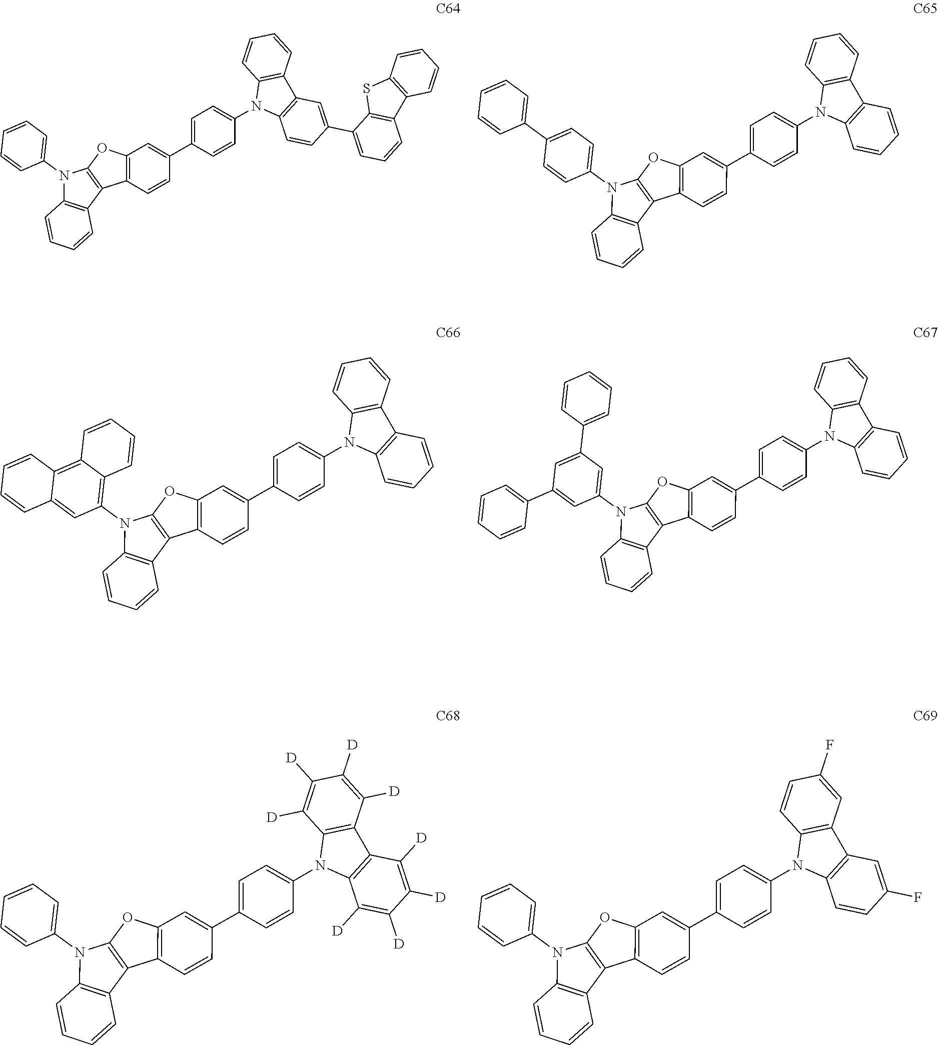 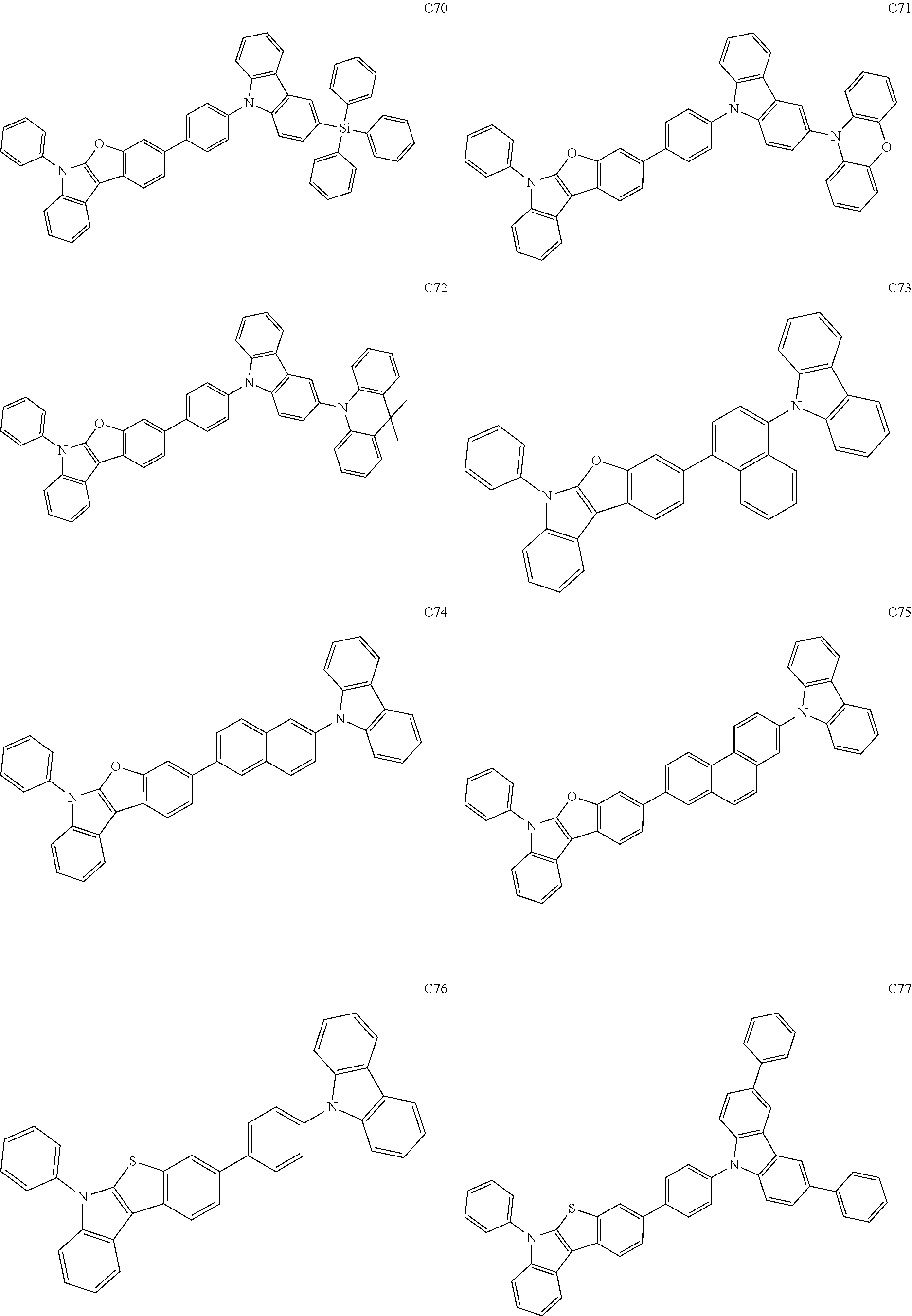 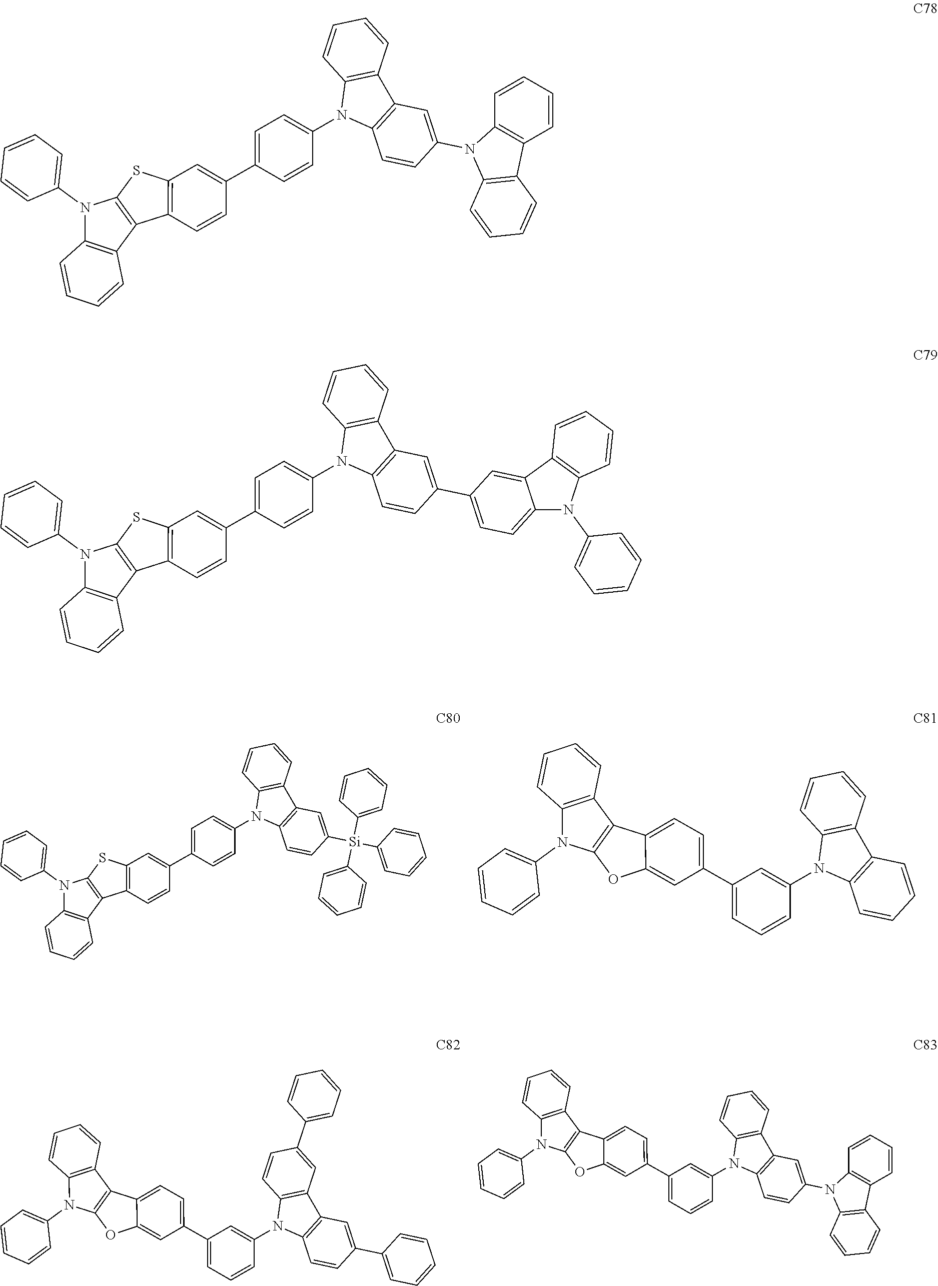 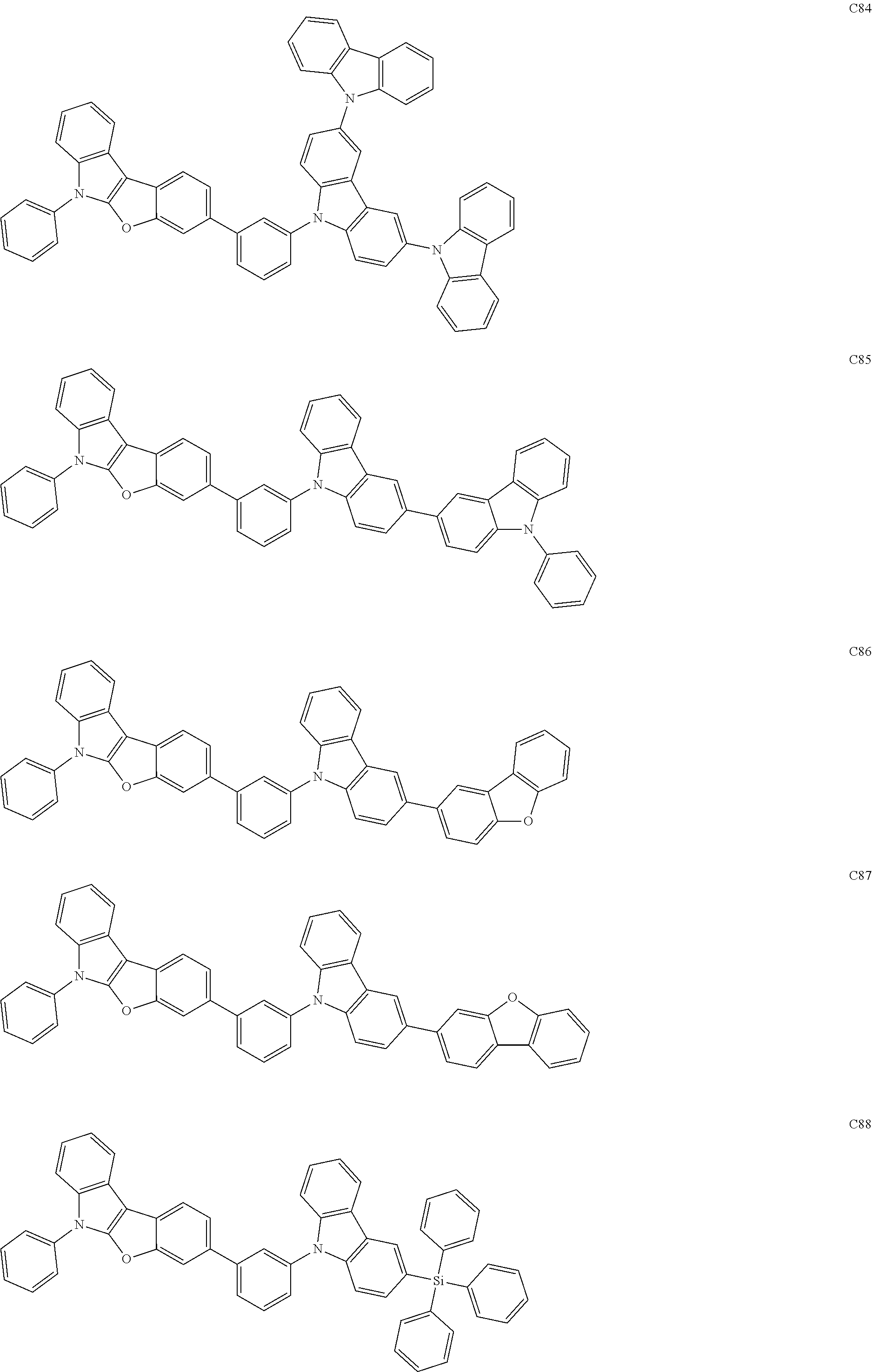 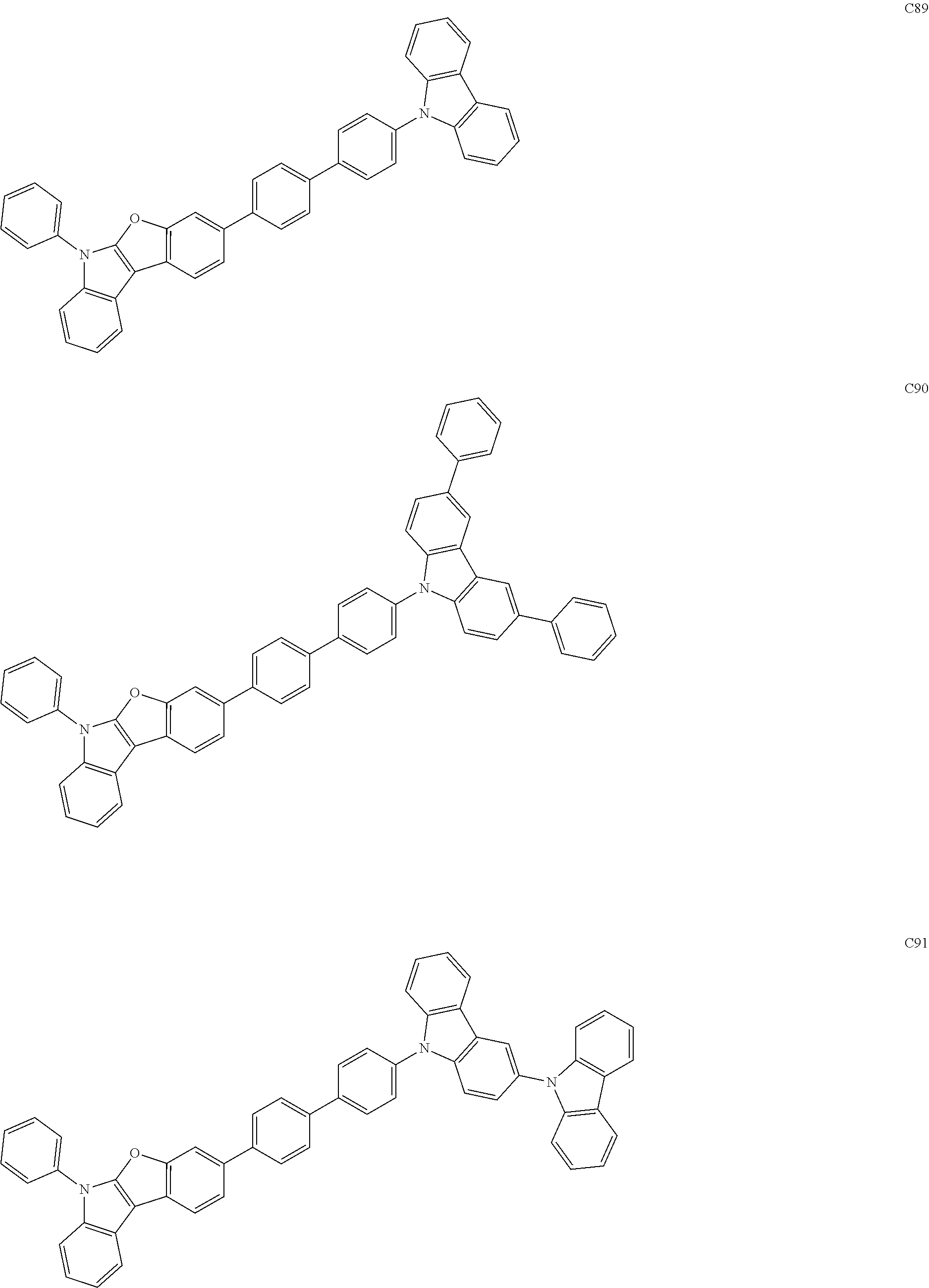 [SMILES: c1ccc(-n2c3ccccc3c3c4ccc(-c5ccc(-c6ccc(-n7c8ccccc8c8ccccc87)cc6)cc5)cc4oc32)cc1 ; c1ccc(-c2ccc3c(c2)c2cc(-c4ccccc4)ccc2n3-c2ccc(-c3ccc(-c4ccc5c(c4)oc4c5c5ccccc5n4-c4ccccc4)cc3)cc2)cc1 ; c1ccc(-n2c3ccccc3c3c4ccc(-c5ccc(-c6ccc(-n7c8ccccc8c8cc(-n9c%10ccccc%10c%10ccccc%109)ccc87)cc6)cc5)cc4oc32)cc1] 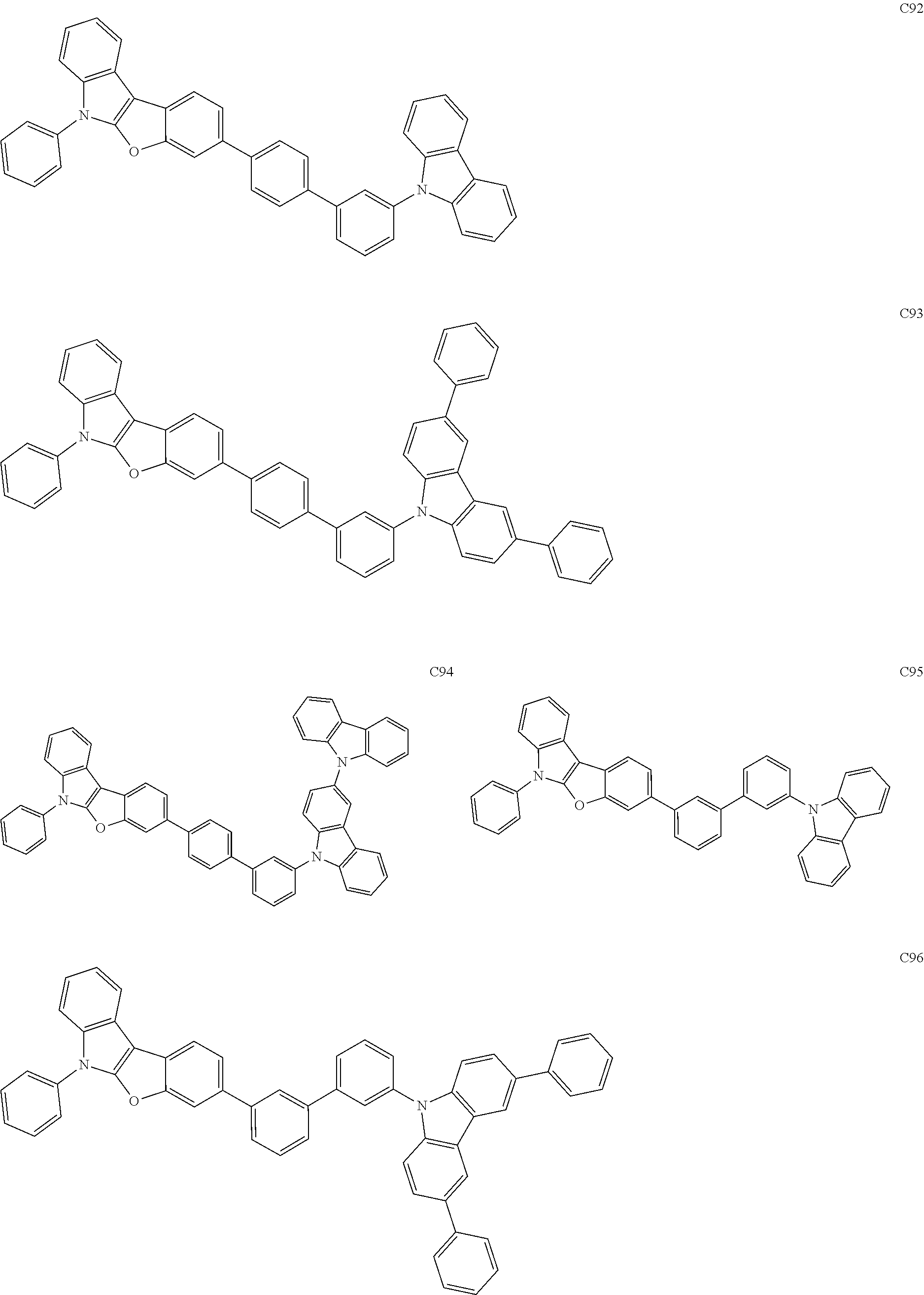 [SMILES: c1ccc(-n2c3ccccc3c3c4ccc(-c5ccc(-c6cccc(-n7c8ccccc8c8ccccc87)c6)cc5)cc4oc32)cc1 ; c1ccc(-c2ccc3c(c2)c2cc(-c4ccccc4)ccc2n3-c2cccc(-c3ccc(-c4ccc5c(c4)oc4c5c5ccccc5n4-c4ccccc4)cc3)c2)cc1 ; c1ccc(-n2c3ccccc3c3c4ccc(-c5ccc(-c6cccc(-n7c8ccccc8c8cc(-n9c%10ccccc%10c%10ccccc%109)ccc87)c6)cc5)cc4oc32)cc1 ; c1ccc(-n2c3ccccc3c3c4ccc(-c5cccc(-c6cccc(-n7c8ccccc8c8ccccc87)c6)c5)cc4oc32)cc1 ; c1ccc(-c2ccc3c(c2)c2cc(-c4ccccc4)ccc2n3-c2cccc(-c3cccc(-c4ccc5c(c4)oc4c5c5ccccc5n4-c4ccccc4)c3)c2)cc1] 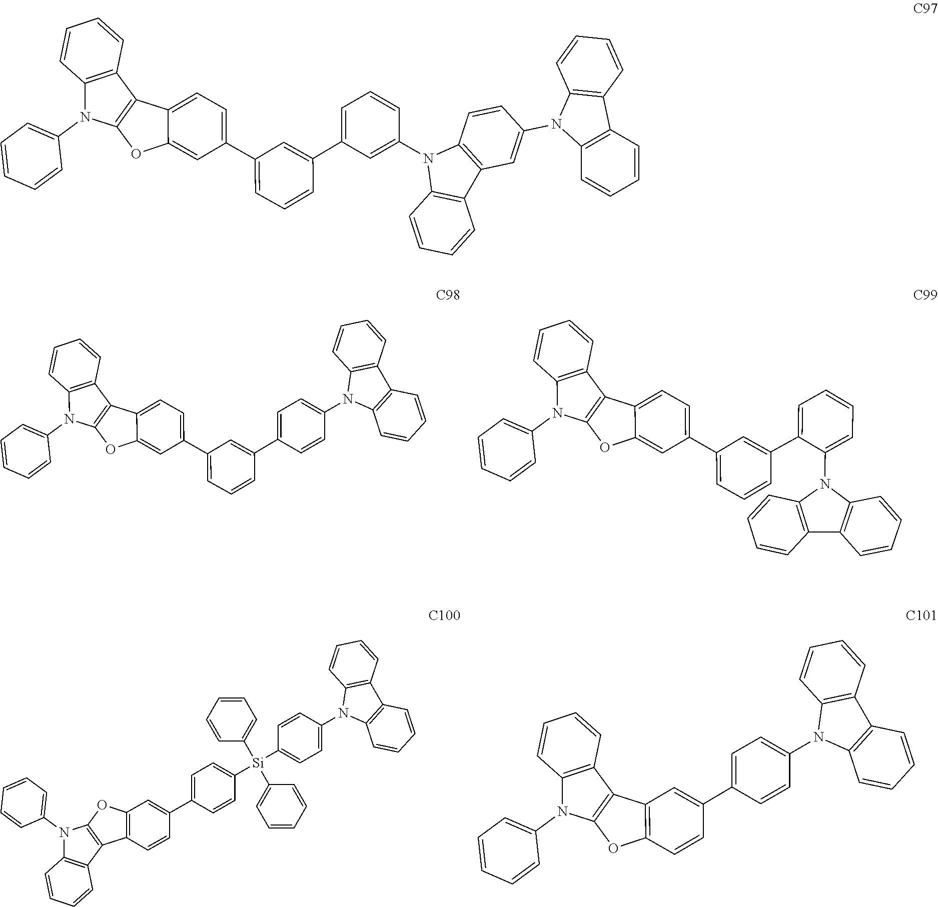 [SMILES: c1ccc(-n2c3ccccc3c3c4ccc(-c5cccc(-c6cccc(-n7c8ccccc8c8cc(-n9c%10ccccc%10c%10ccccc%109)ccc87)c6)c5)cc4oc32)cc1 ; c1ccc(-n2c3ccccc3c3c4ccc(-c5cccc(-c6ccc(-n7c8ccccc8c8ccccc87)cc6)c5)cc4oc32)cc1 ; c1ccc(-n2c3ccccc3c3c4ccc(-c5cccc(-c6ccccc6-n6c7ccccc7c7ccccc76)c5)cc4oc32)cc1 ; c1ccc(-n2c3ccccc3c3c4ccc(-c5ccc([Si](c6ccccc6)(c6ccccc6)c6ccc(-n7c8ccccc8c8ccccc87)cc6)cc5)cc4oc32)cc1 ; c1ccc(-n2c3ccccc3c3c4cc(-c5ccc(-n6c7ccccc7c7ccccc76)cc5)ccc4oc32)cc1] 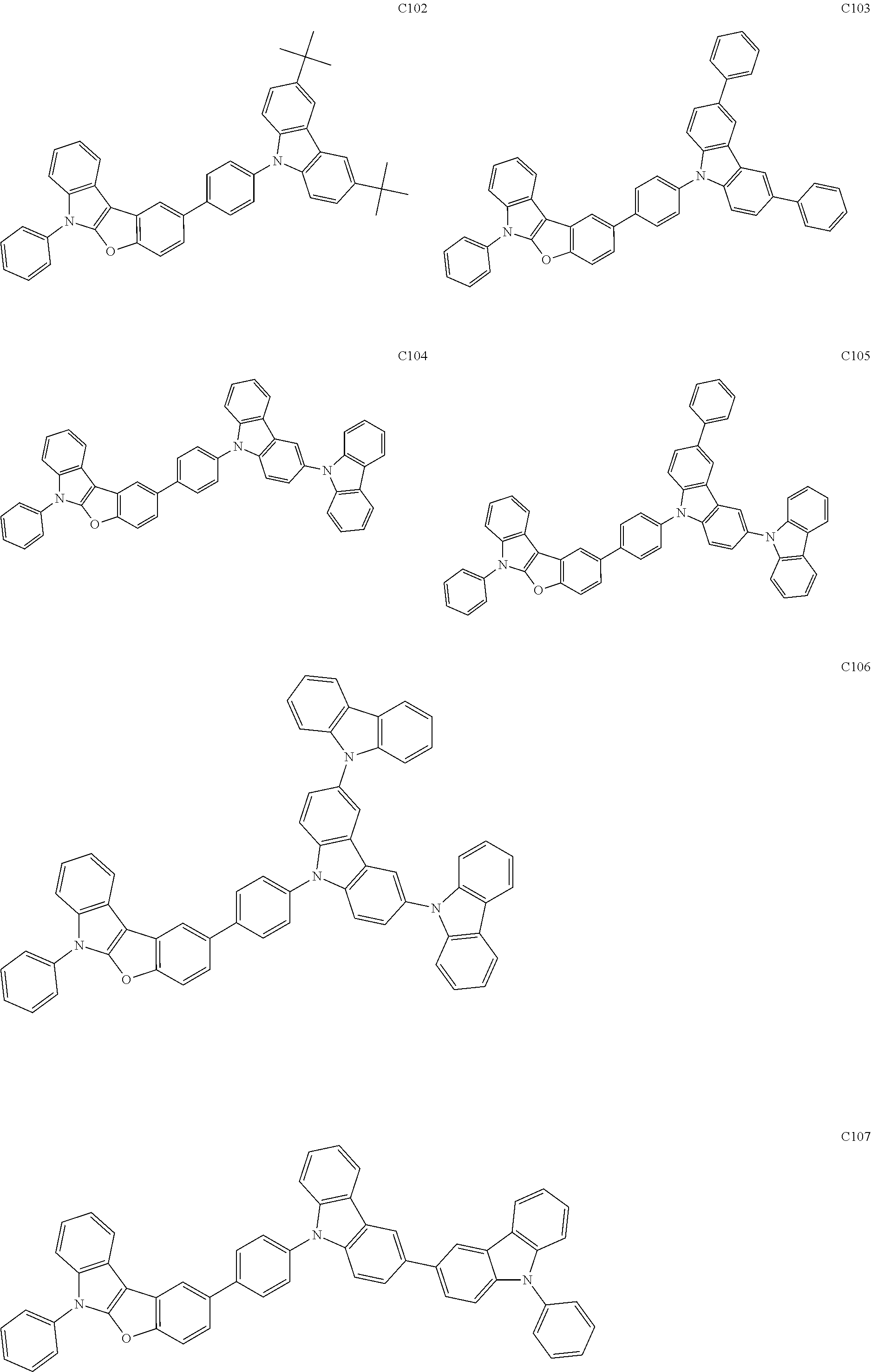 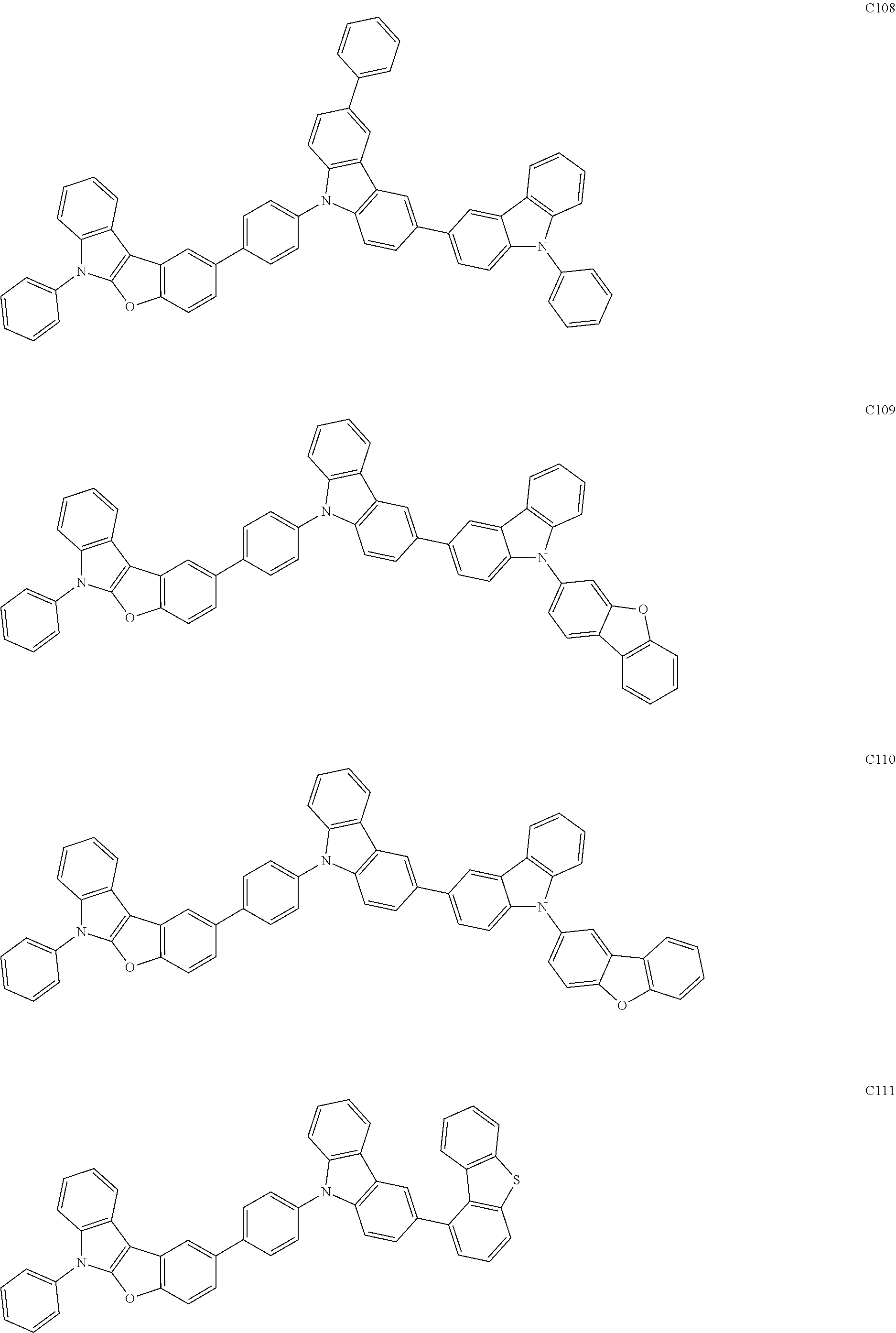 [SMILES: c1ccc(-c2ccc3c(c2)c2cc(-c4ccc5c(c4)c4ccccc4n5-c4ccccc4)ccc2n3-c2ccc(-c3ccc4oc5c(c4c3)c3ccccc3n5-c3ccccc3)cc2)cc1 ; c1ccc(-n2c3ccccc3c3c4cc(-c5ccc(-n6c7ccccc7c7cc(-c8ccc9c(c8)c8ccccc8n9-c8ccc9c(c8)oc8ccccc89)ccc76)cc5)ccc4oc32)cc1 ; c1ccc(-n2c3ccccc3c3c4cc(-c5ccc(-n6c7ccccc7c7cc(-c8ccc9c(c8)c8ccccc8n9-c8ccc9oc%10ccccc%10c9c8)ccc76)cc5)ccc4oc32)cc1 ; c1ccc(-n2c3ccccc3c3c4cc(-c5ccc(-n6c7ccccc7c7cc(-c8cccc9sc%10ccccc%10c89)ccc76)cc5)ccc4oc32)cc1] 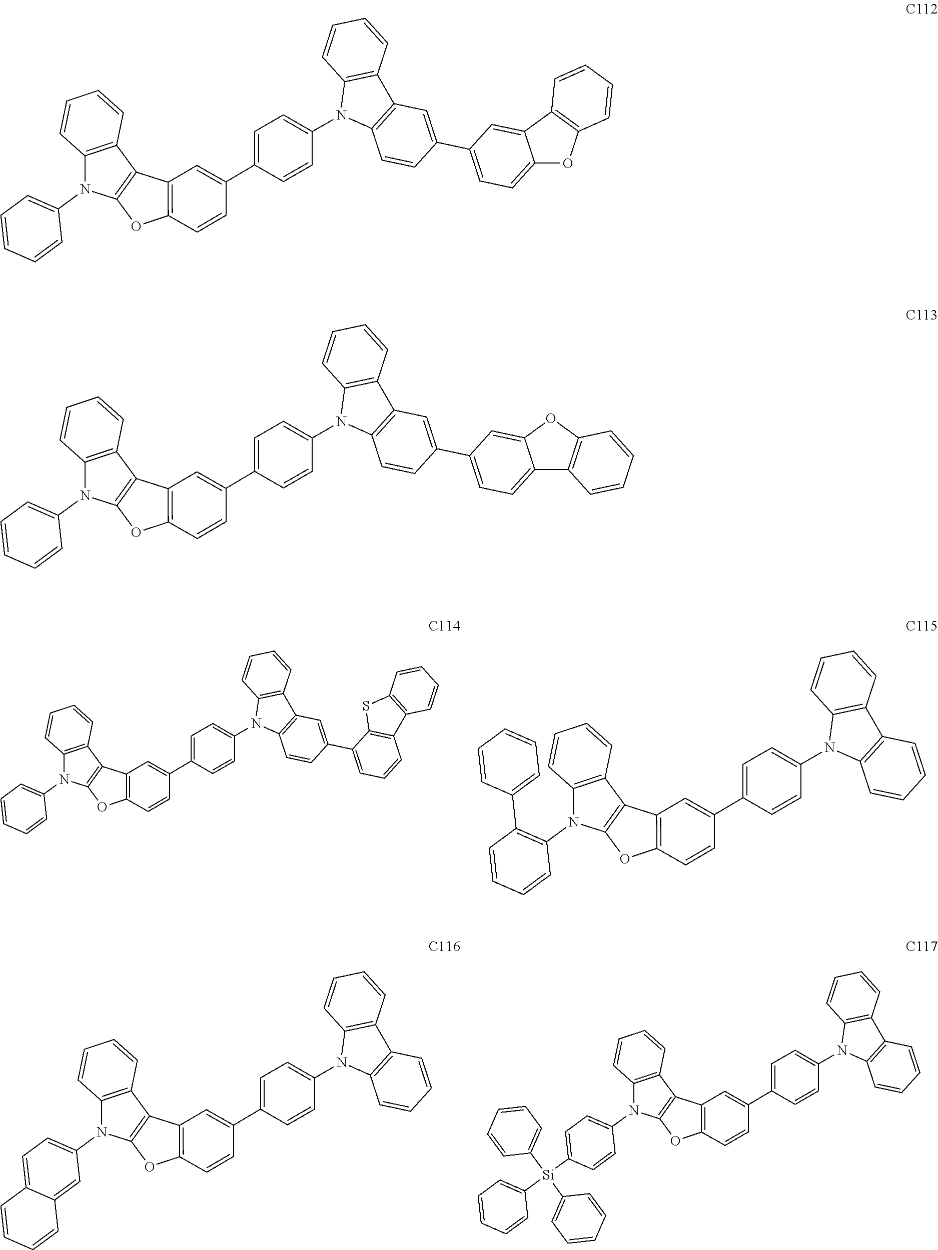 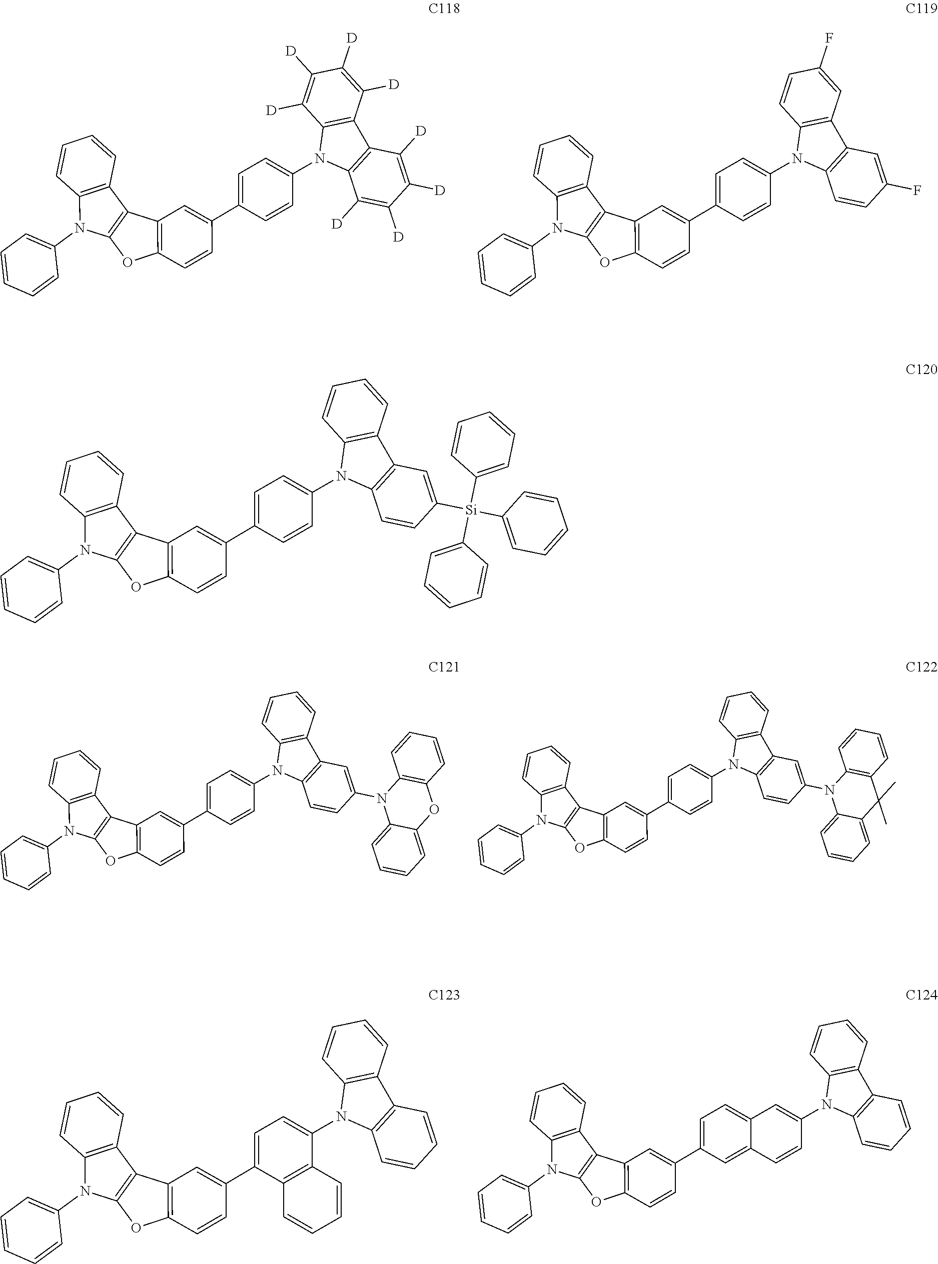 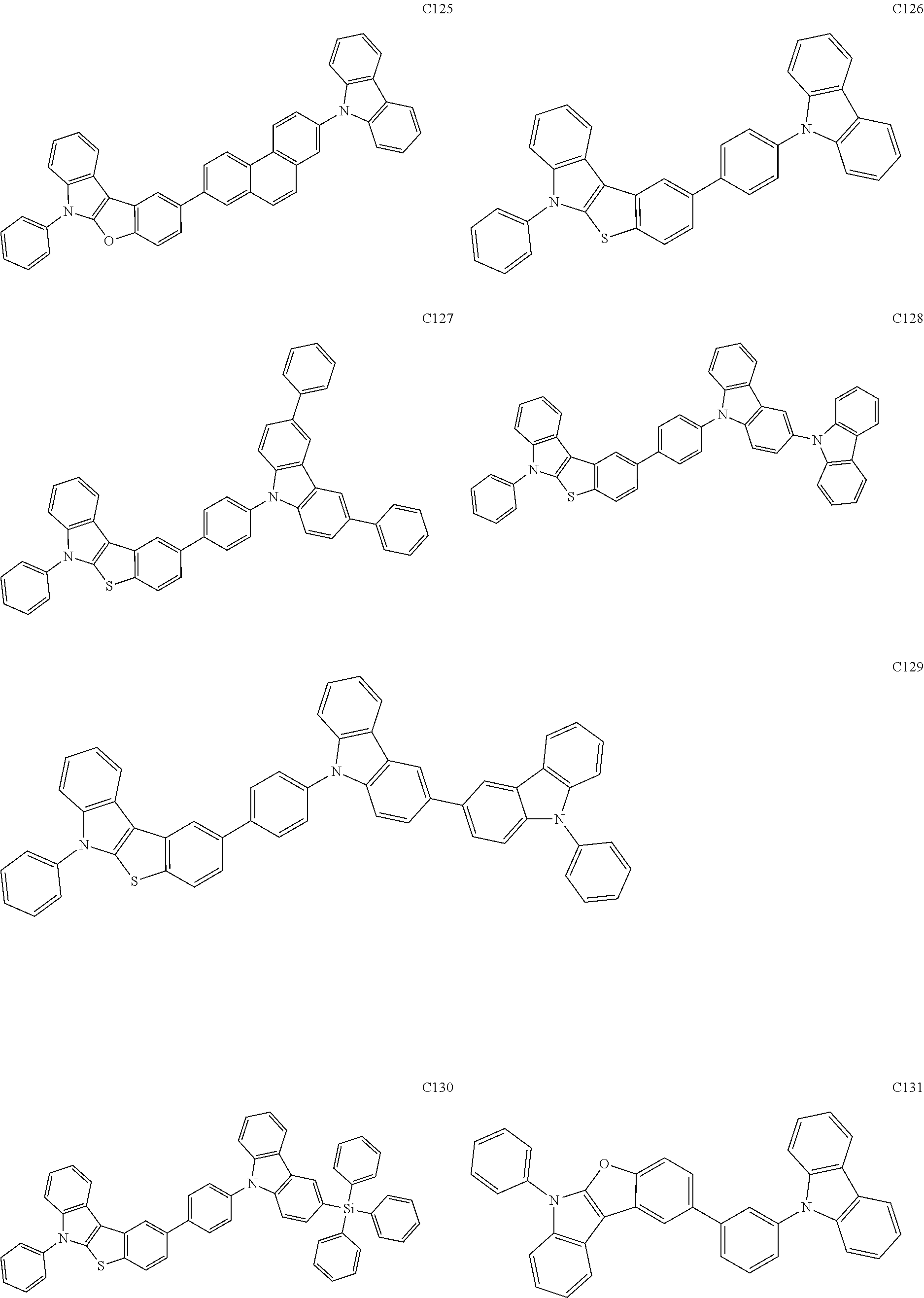 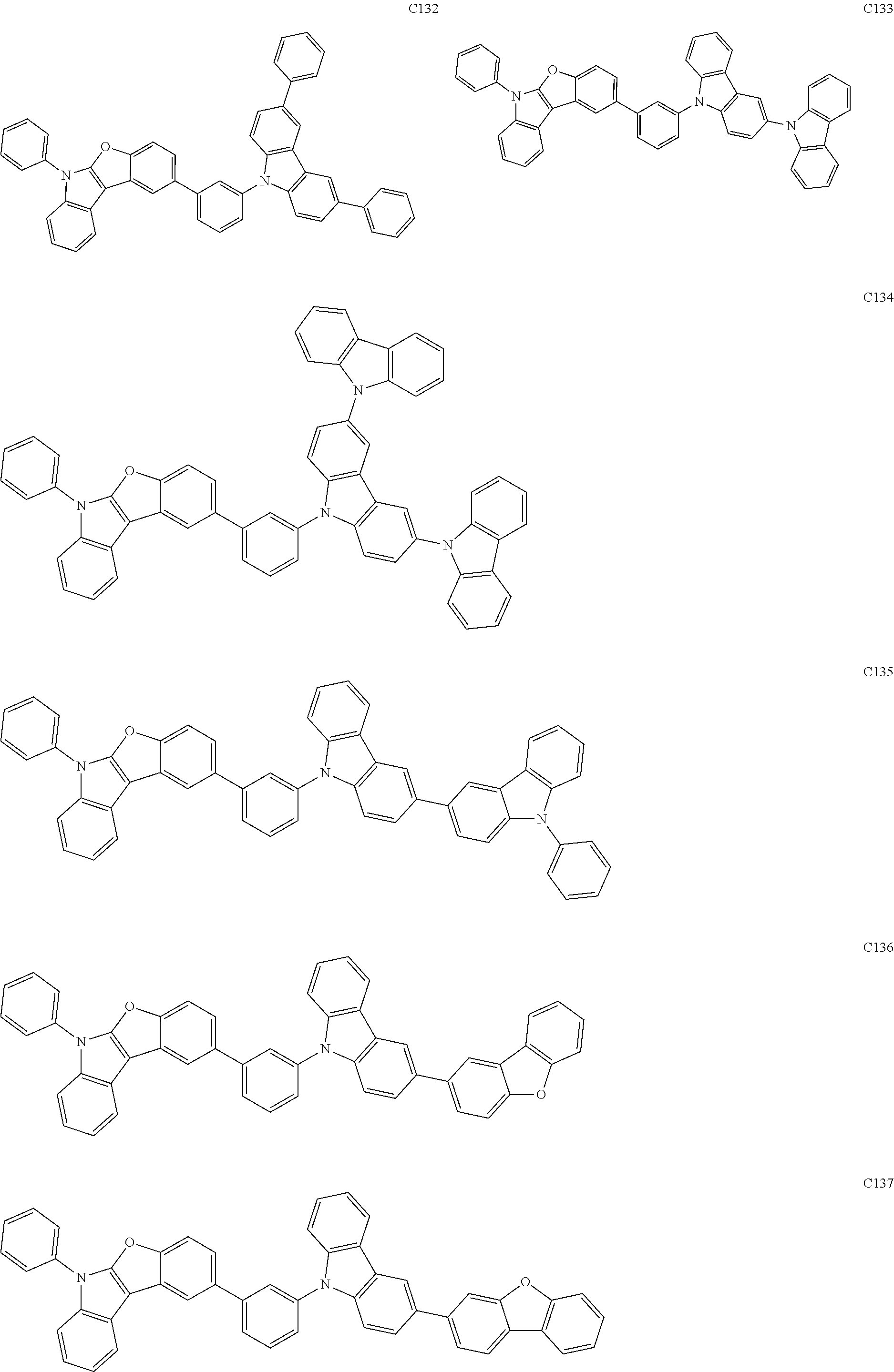 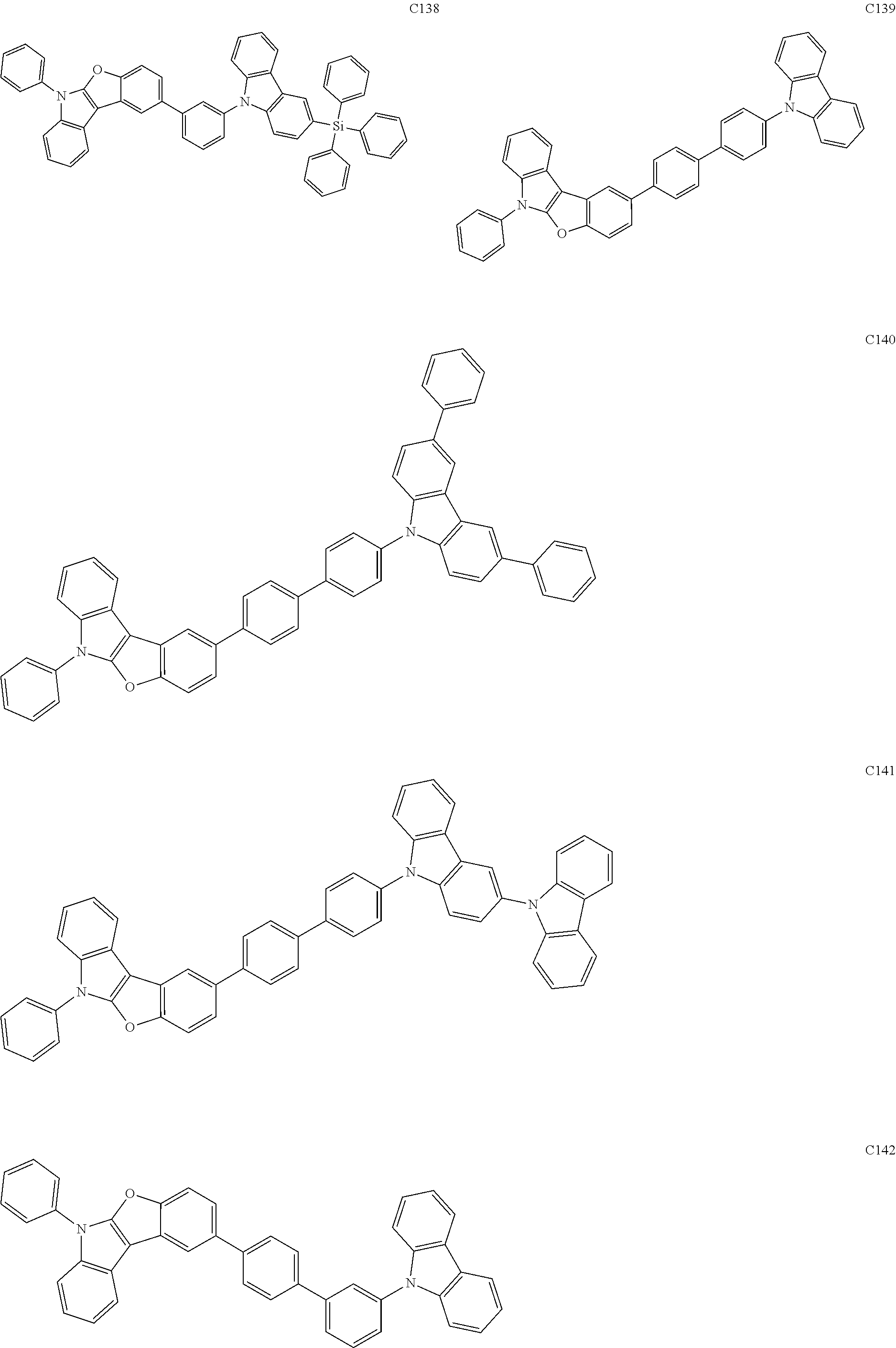 [SMILES: c1ccc(-n2c3ccccc3c3c4cc(-c5cccc(-n6c7ccccc7c7cc([Si](c8ccccc8)(c8ccccc8)c8ccccc8)ccc76)c5)ccc4oc32)cc1 ; c1ccc(-n2c3ccccc3c3c4cc(-c5ccc(-c6ccc(-n7c8ccccc8c8ccccc87)cc6)cc5)ccc4oc32)cc1 ; c1ccc(-c2ccc3c(c2)c2cc(-c4ccccc4)ccc2n3-c2ccc(-c3ccc(-c4ccc5oc6c(c5c4)c4ccccc4n6-c4ccccc4)cc3)cc2)cc1 ; c1ccc(-n2c3ccccc3c3c4cc(-c5ccc(-c6ccc(-n7c8ccccc8c8cc(-n9c%10ccccc%10c%10ccccc%109)ccc87)cc6)cc5)ccc4oc32)cc1 ; c1ccc(-n2c3ccccc3c3c4cc(-c5ccc(-c6cccc(-n7c8ccccc8c8ccccc87)c6)cc5)ccc4oc32)cc1] 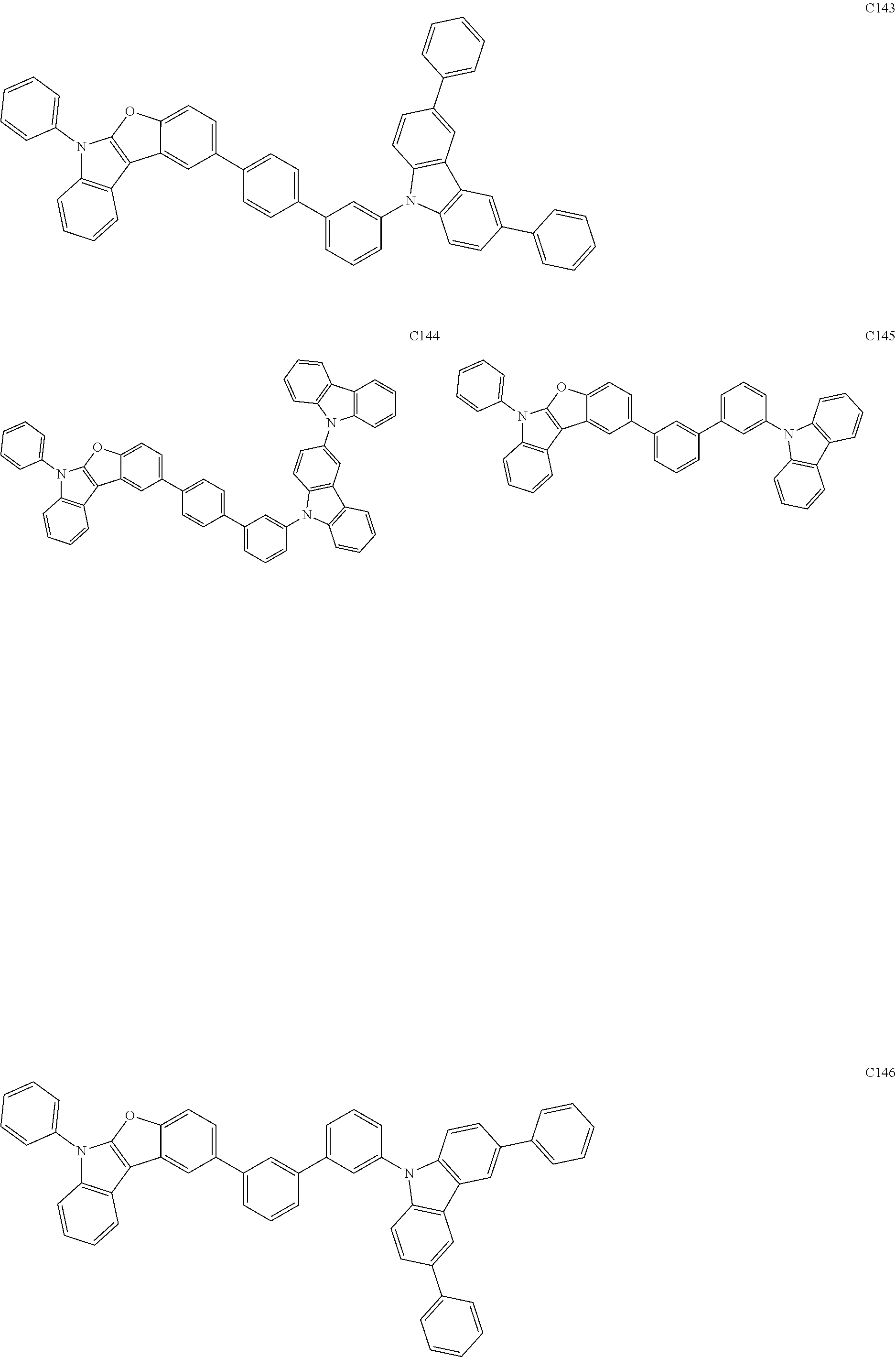 [SMILES: c1ccc(-c2ccc3c(c2)c2cc(-c4ccccc4)ccc2n3-c2cccc(-c3ccc(-c4ccc5oc6c(c5c4)c4ccccc4n6-c4ccccc4)cc3)c2)cc1 ; c1ccc(-n2c3ccccc3c3c4cc(-c5ccc(-c6cccc(-n7c8ccccc8c8cc(-n9c%10ccccc%10c%10ccccc%109)ccc87)c6)cc5)ccc4oc32)cc1 ; c1ccc(-n2c3ccccc3c3c4cc(-c5cccc(-c6cccc(-n7c8ccccc8c8ccccc87)c6)c5)ccc4oc32)cc1 ; c1ccc(-c2ccc3c(c2)c2cc(-c4ccccc4)ccc2n3-c2cccc(-c3cccc(-c4ccc5oc6c(c5c4)c4ccccc4n6-c4ccccc4)c3)c2)cc1] 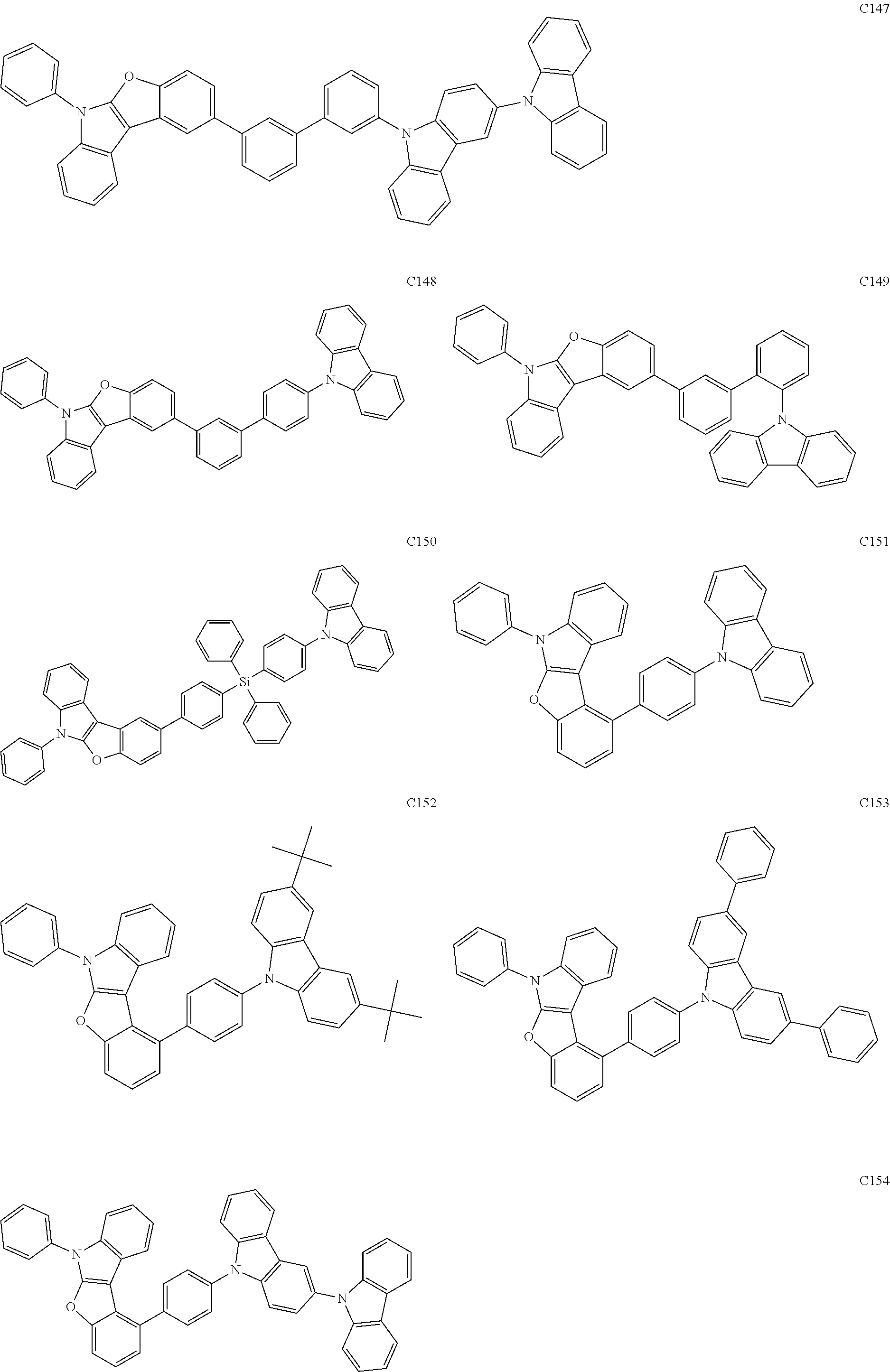 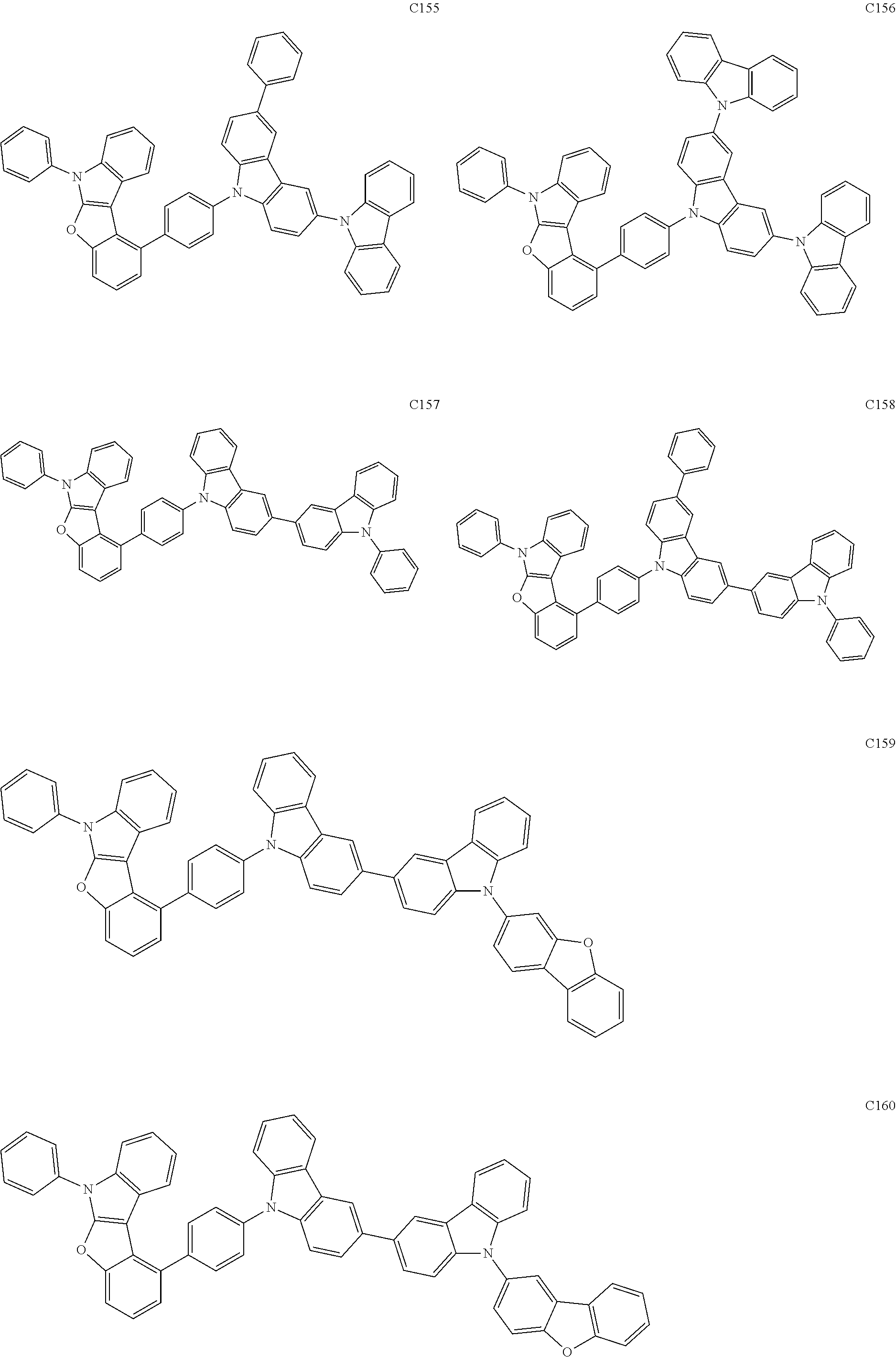 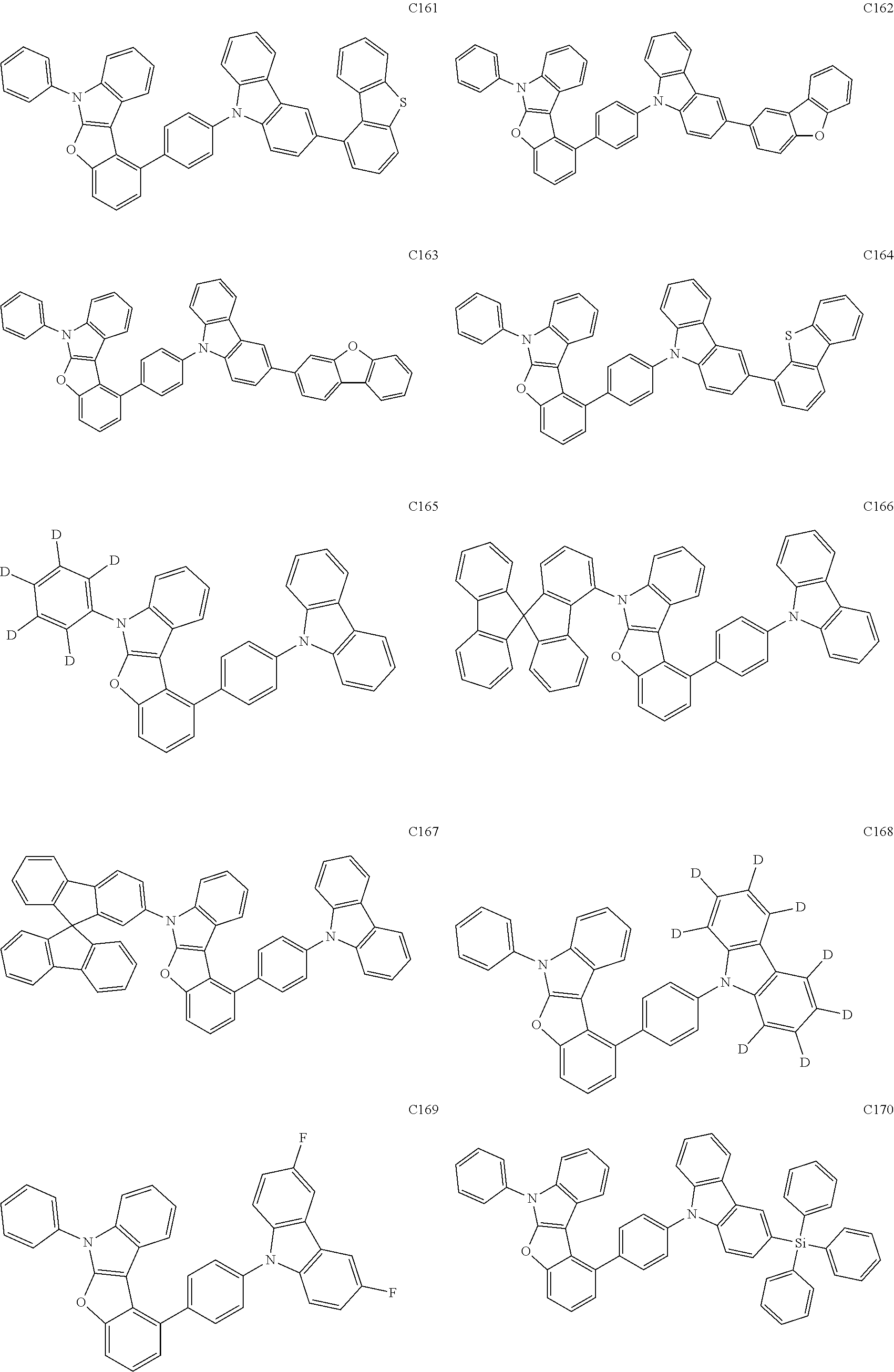 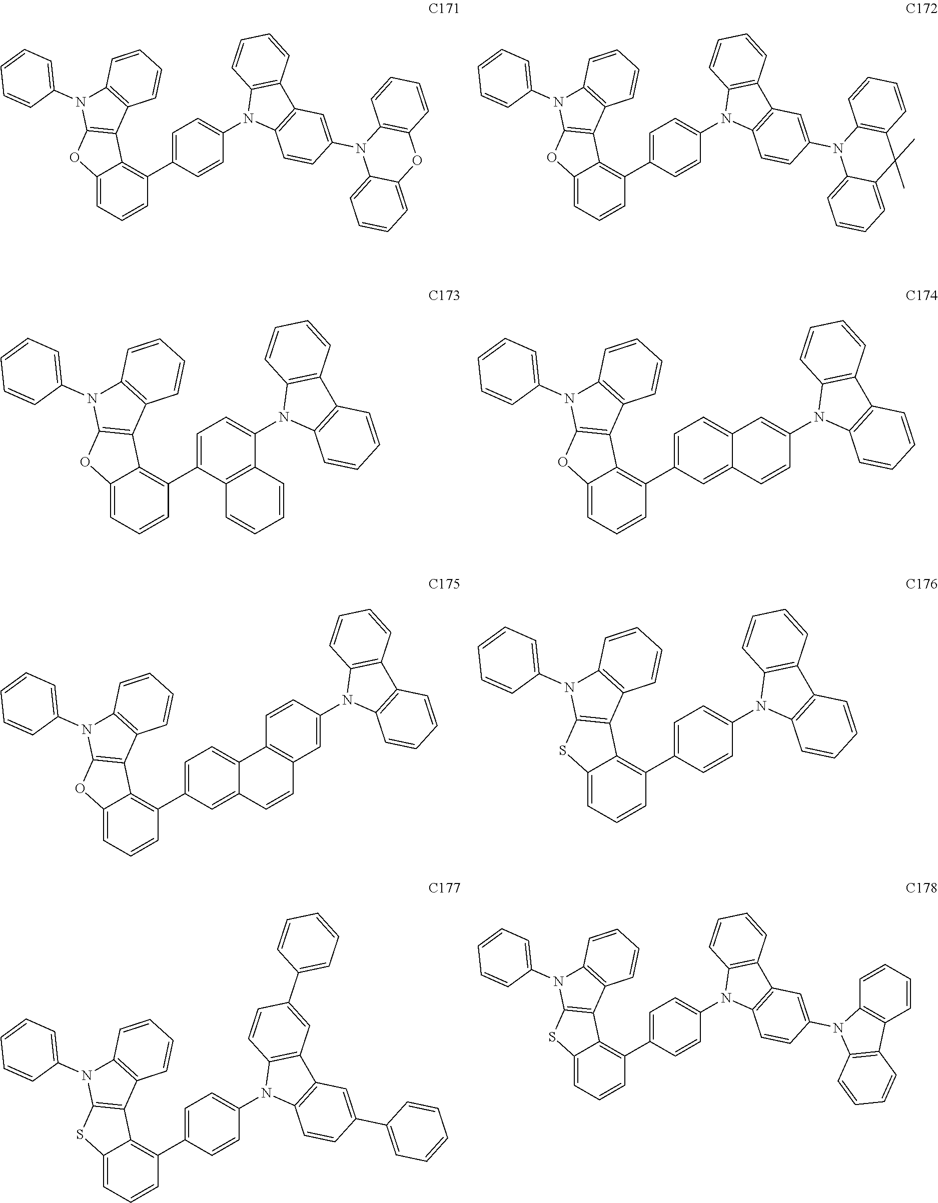 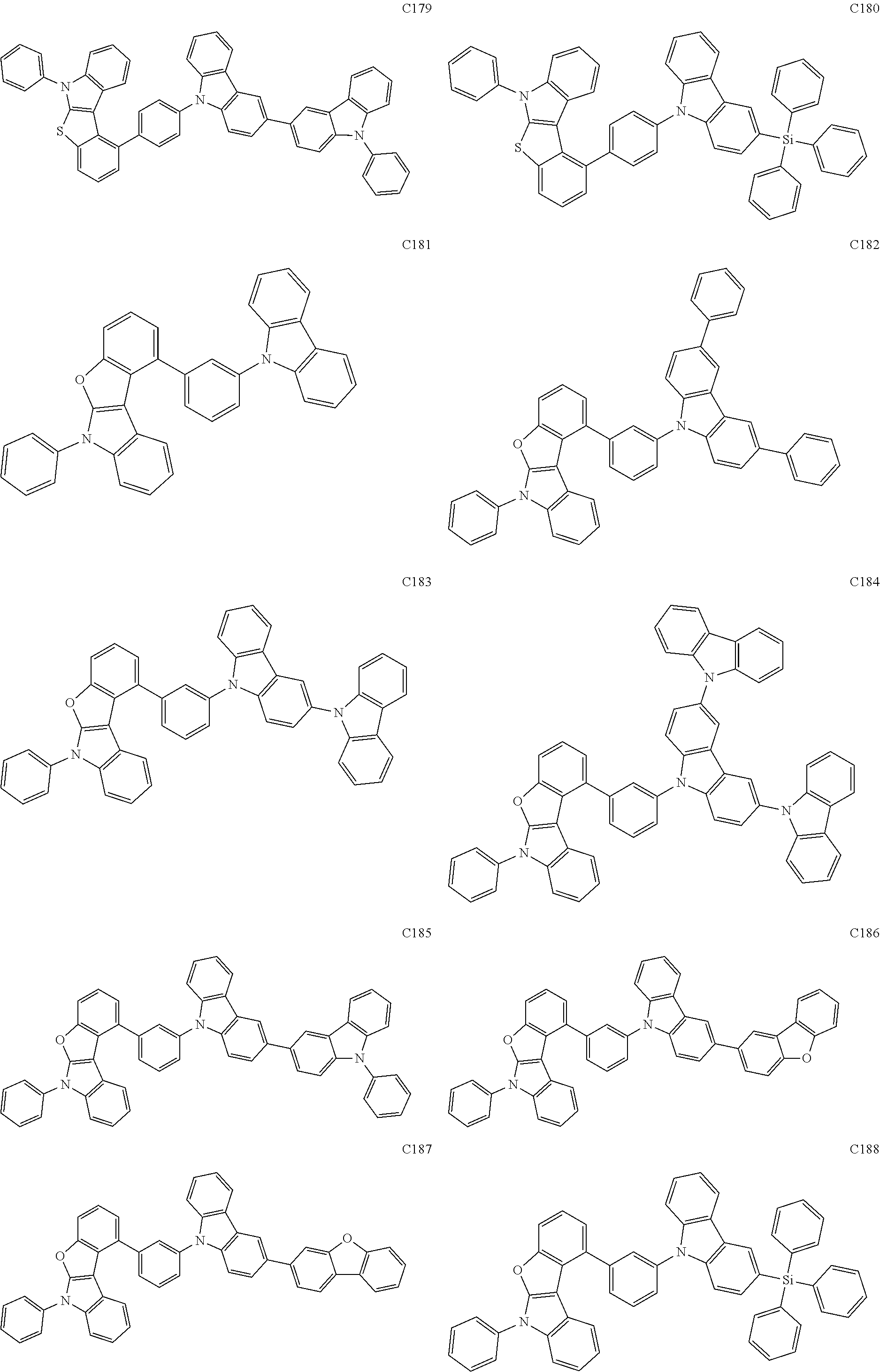 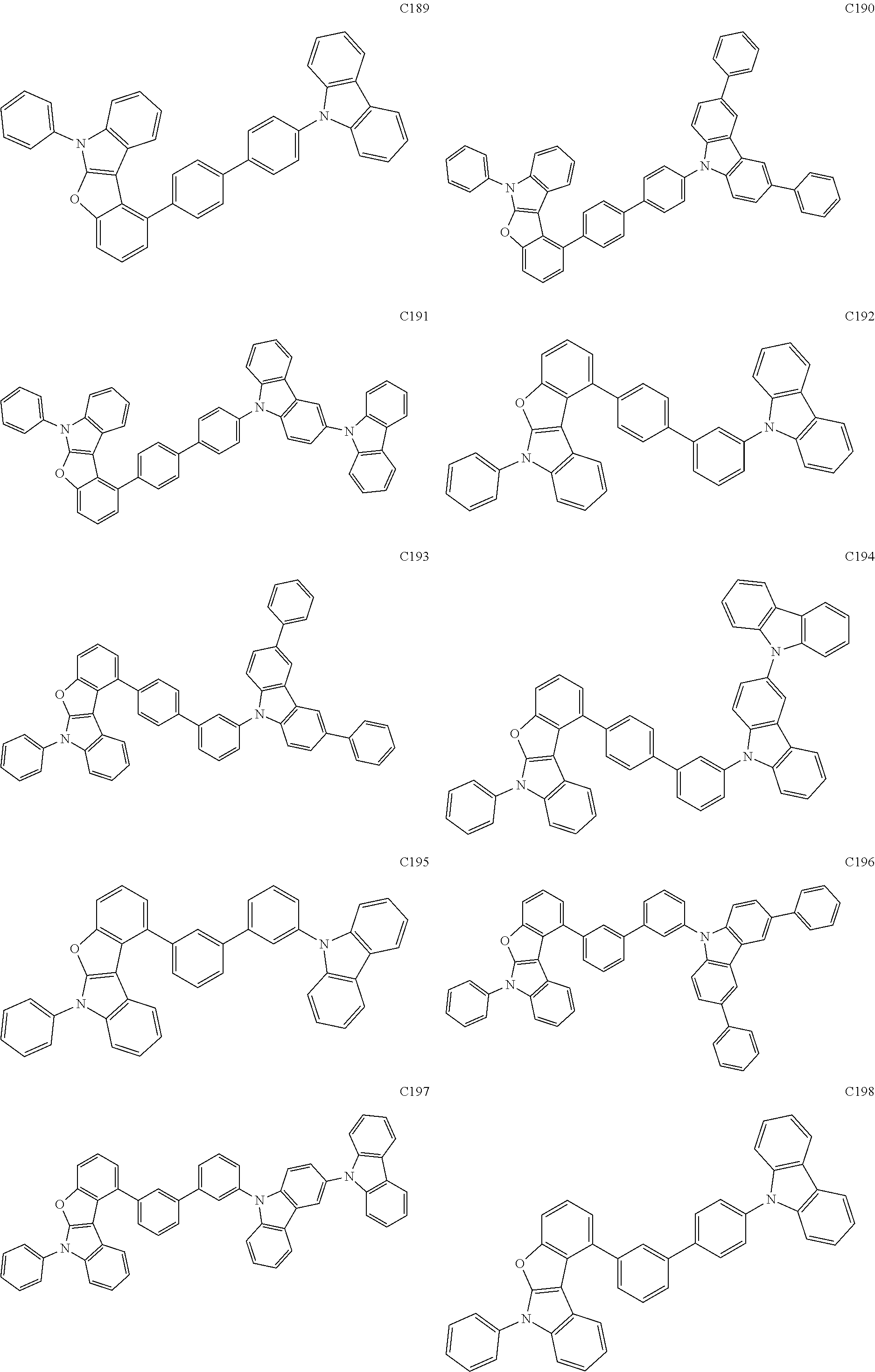 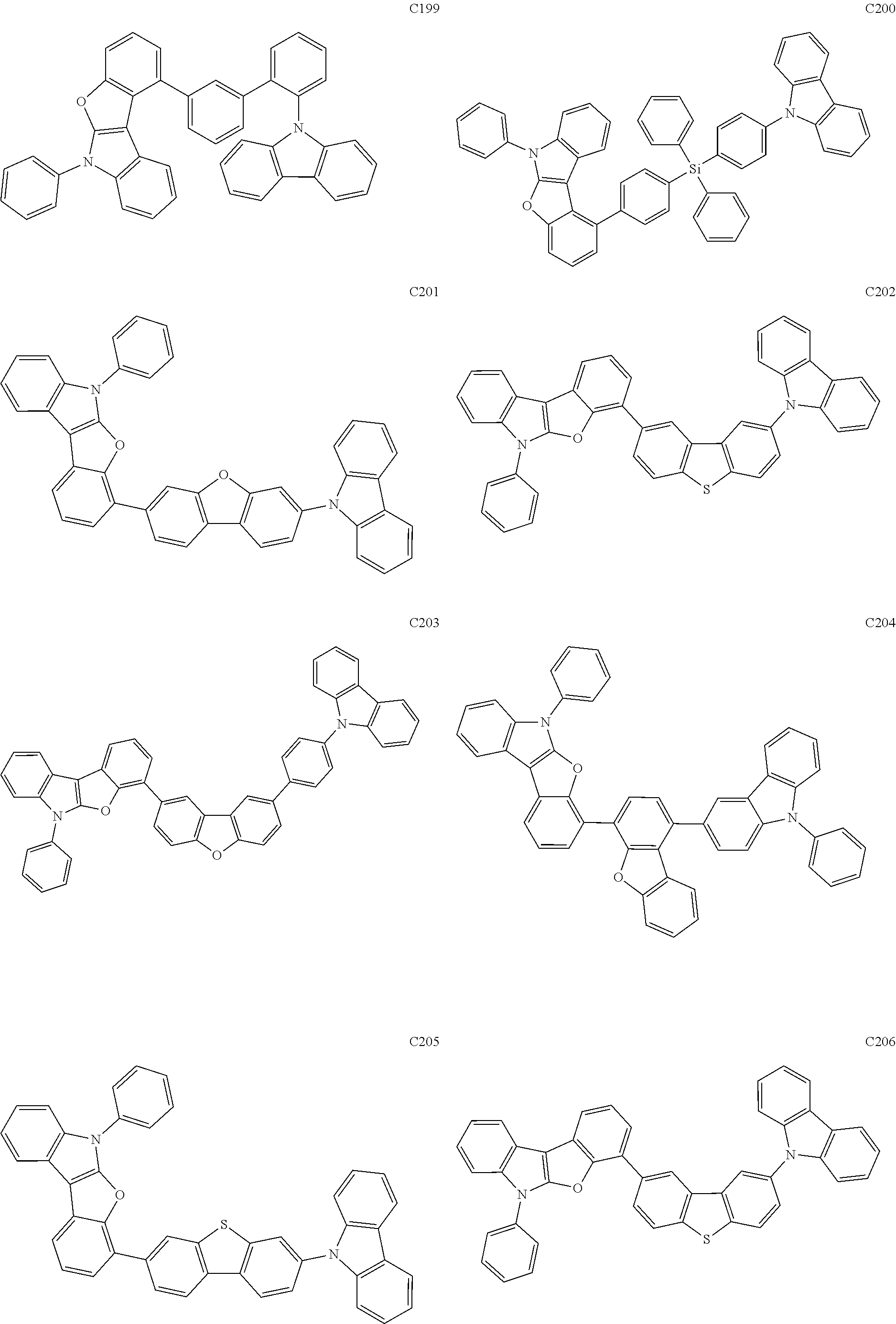 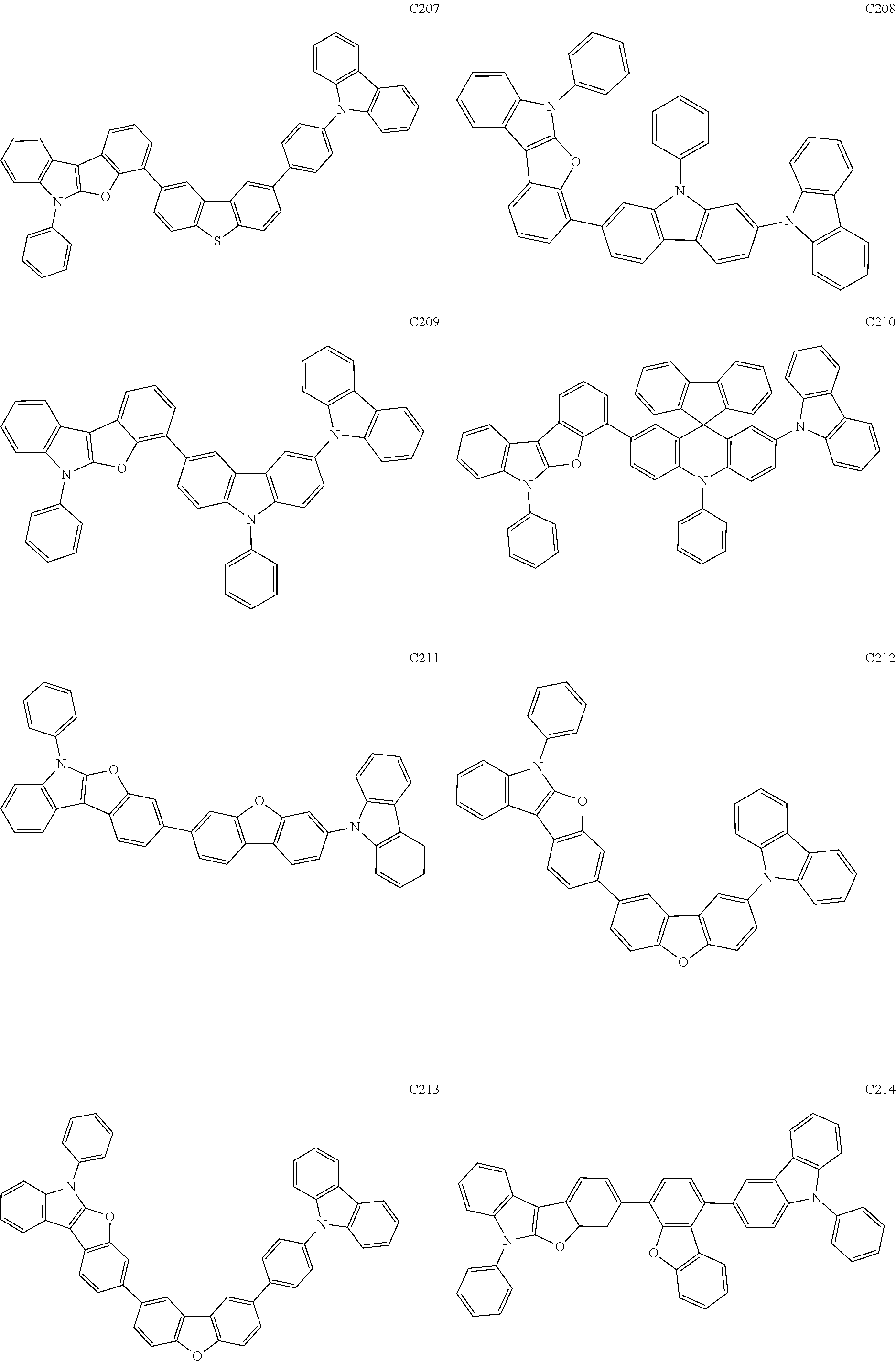 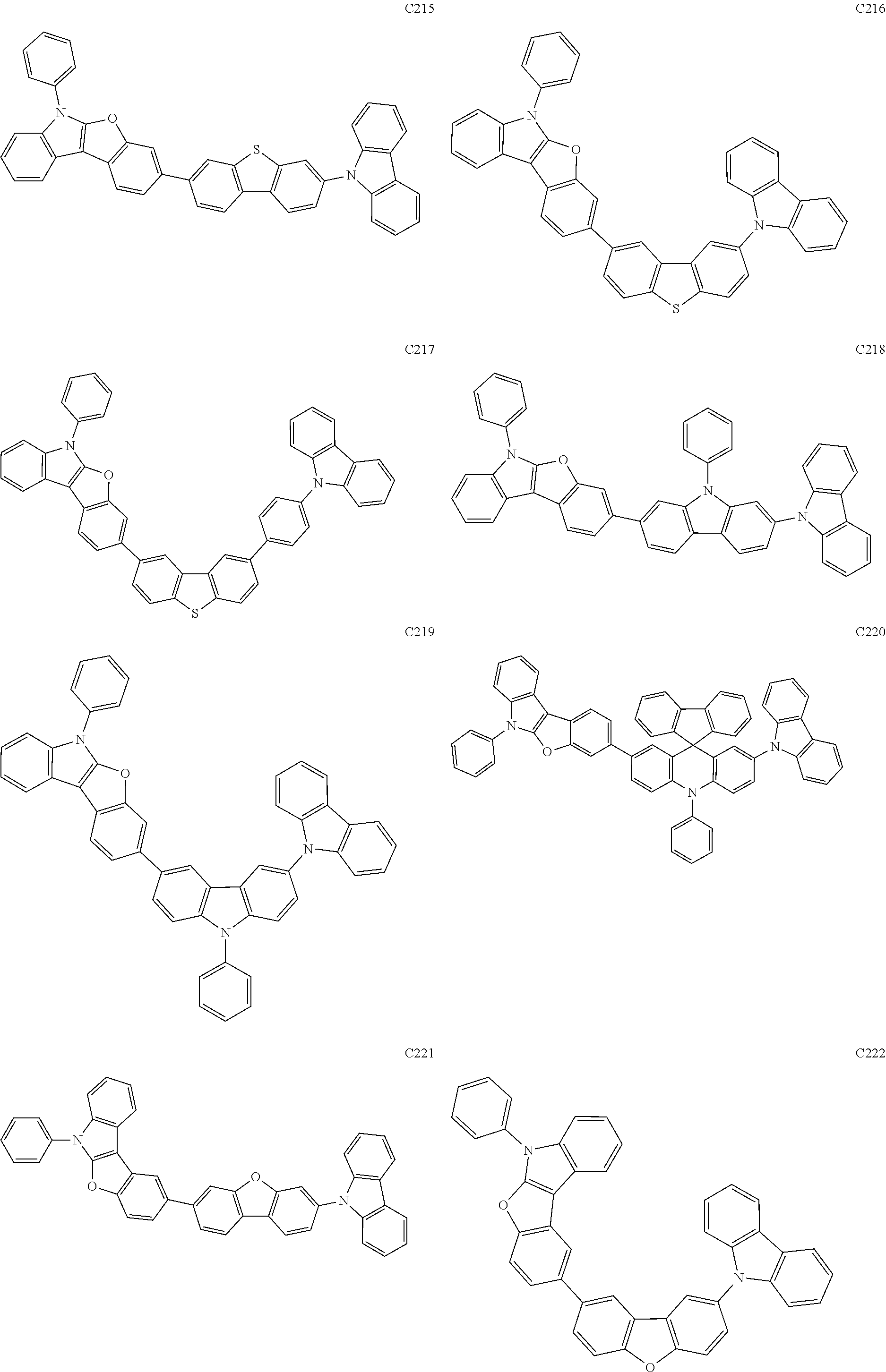 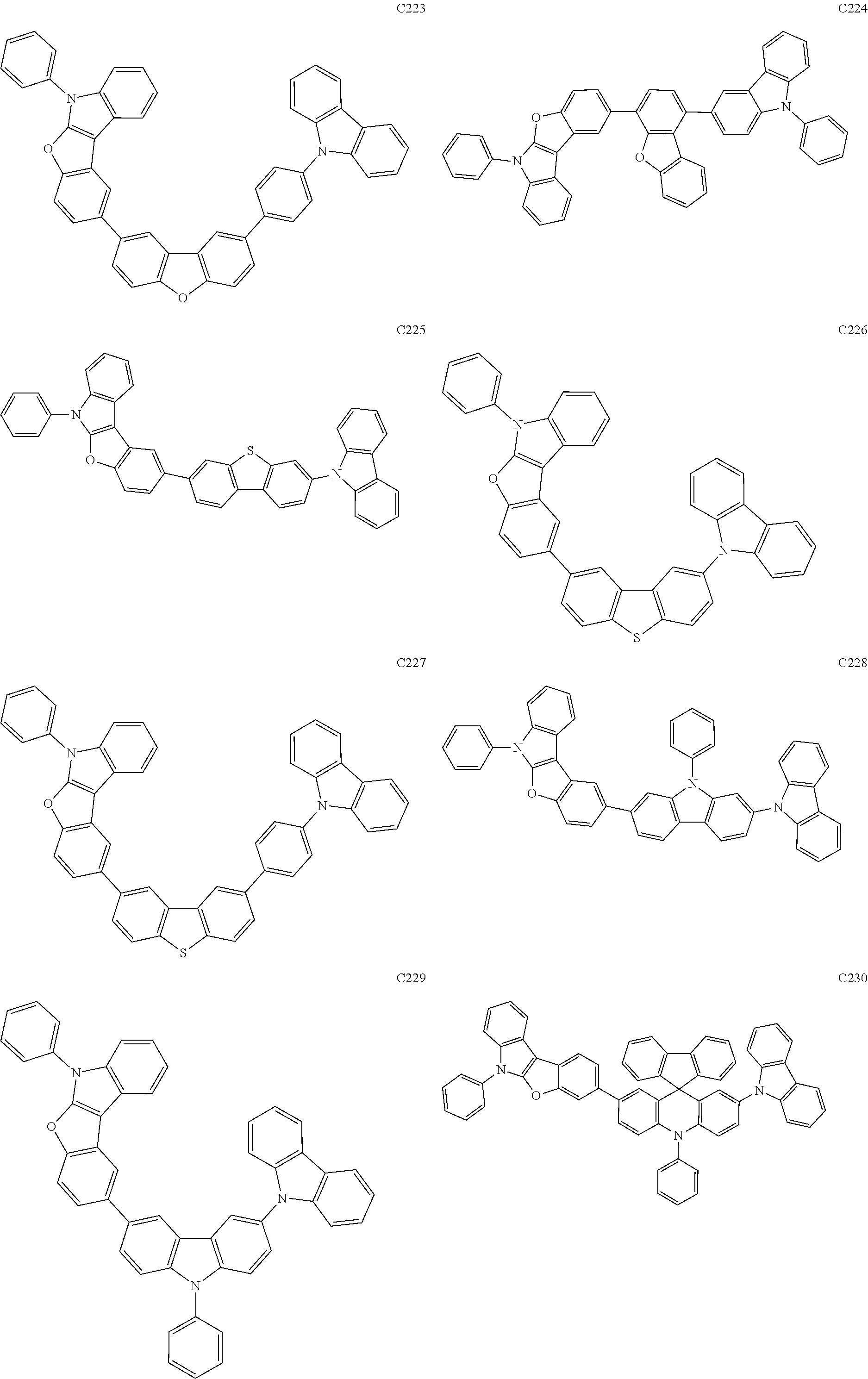 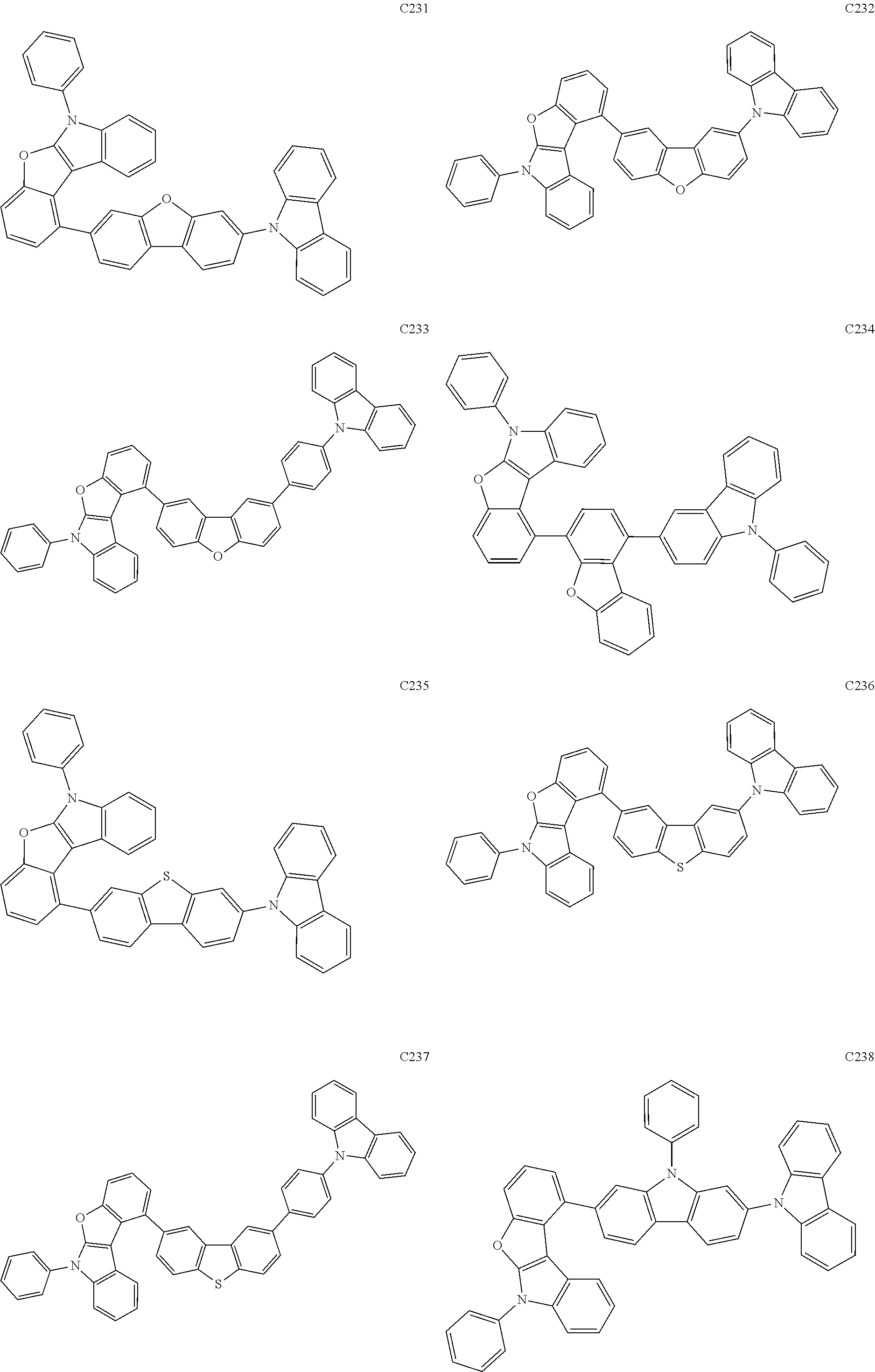 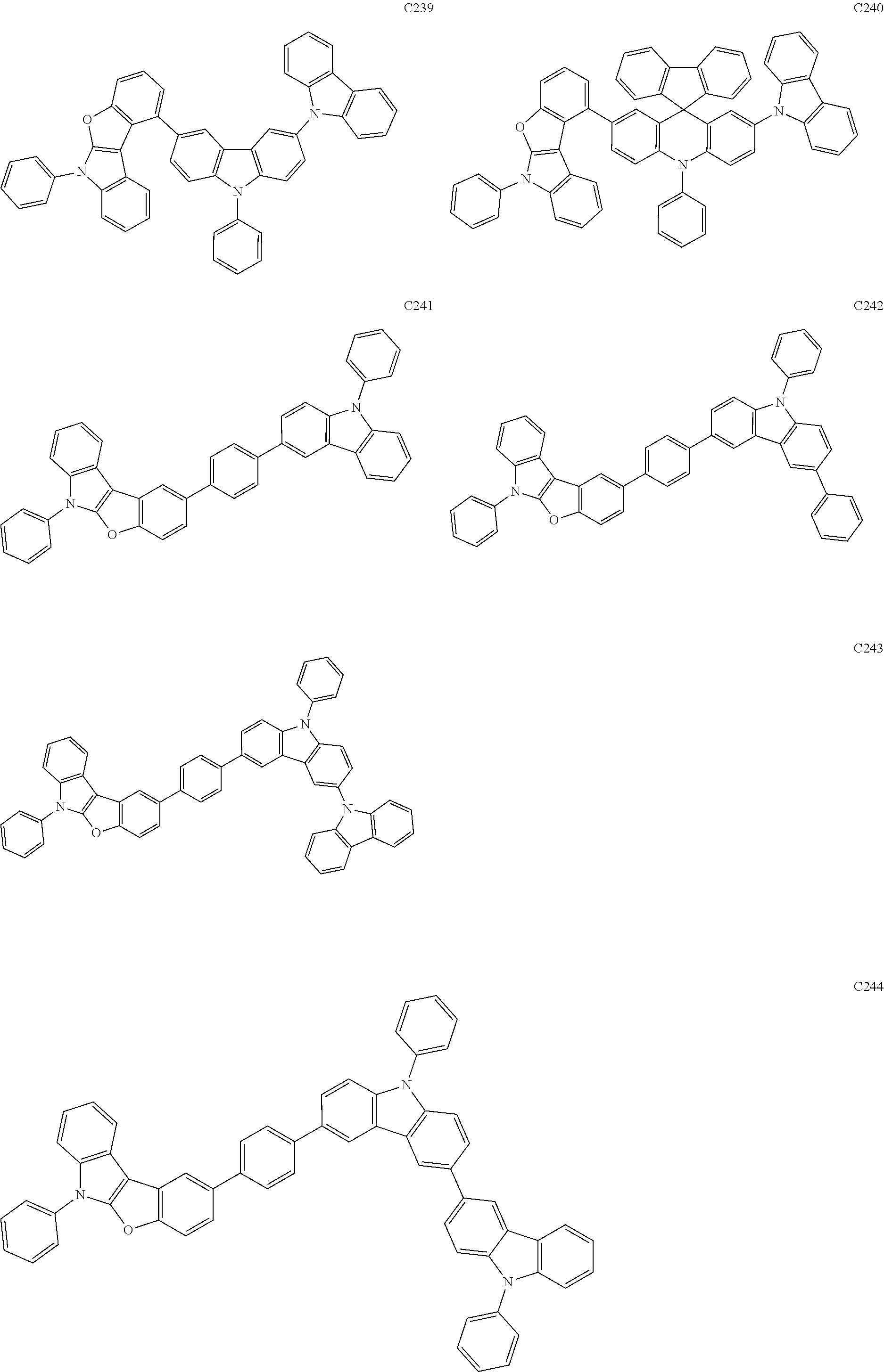 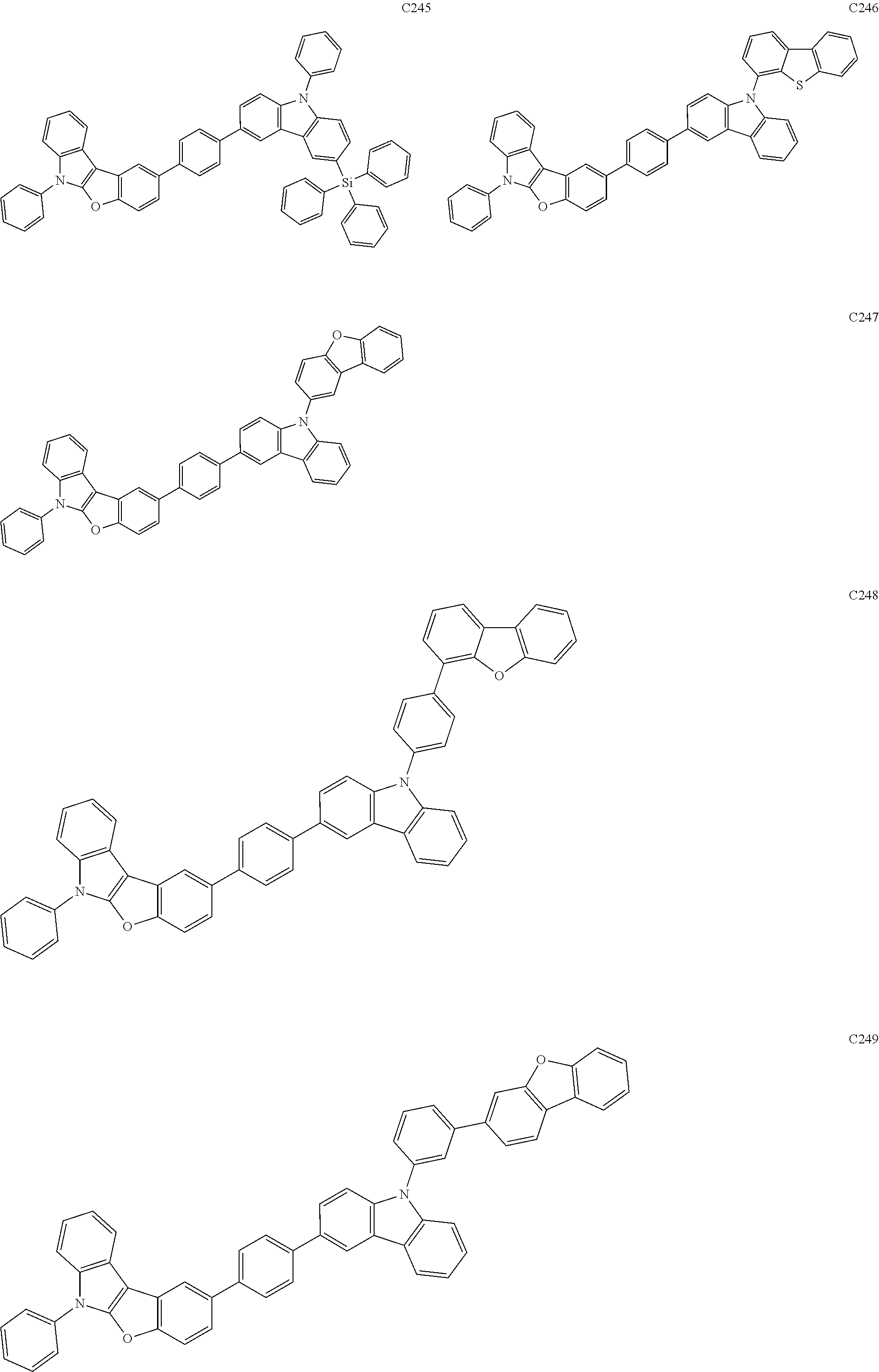 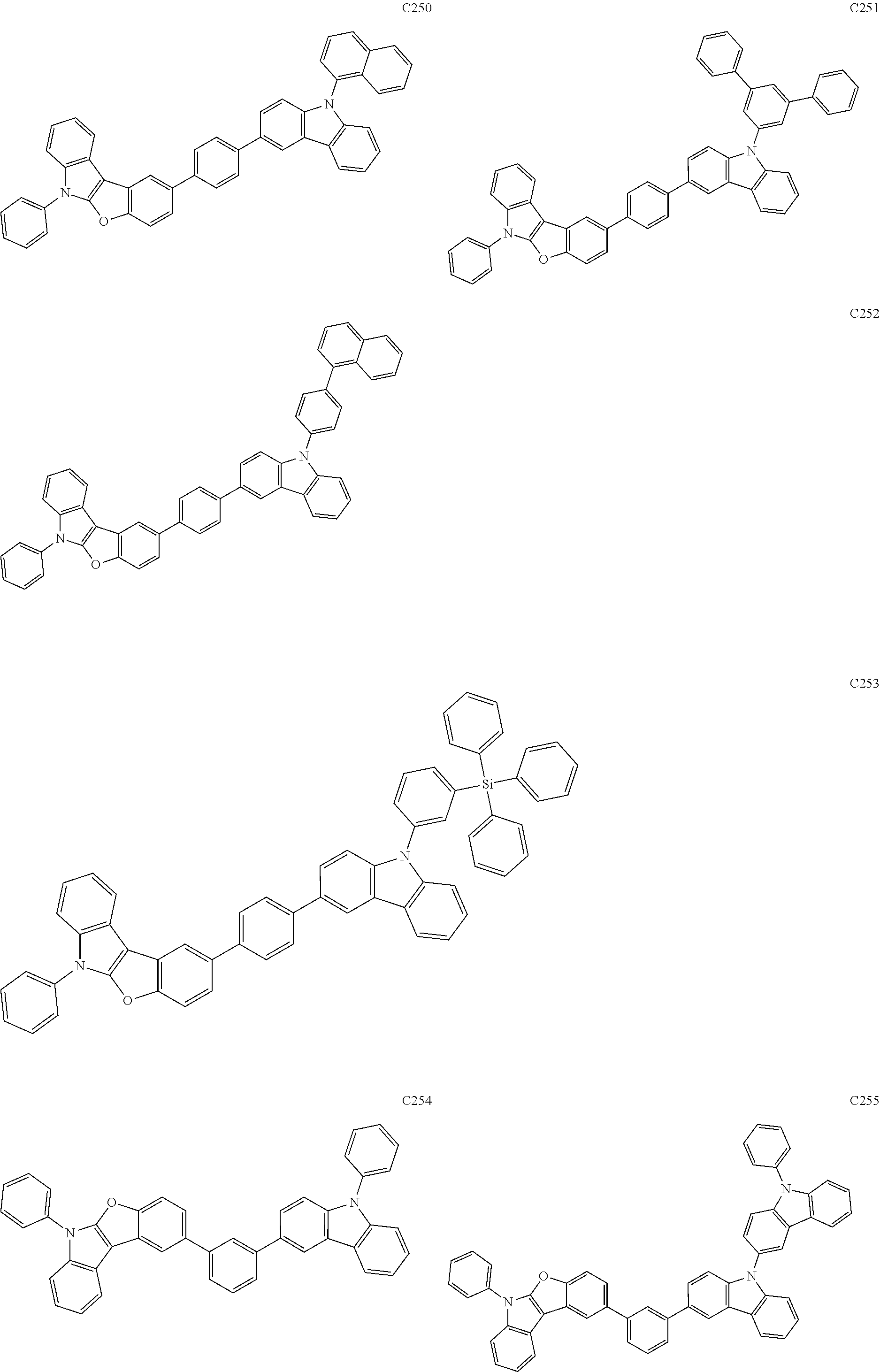 [chemical structure]
